(19) United States
(12) Patent Application Publication
(10) Pub. No.: US 2022/0411419 A1
(43) Pub. Date: Dec. 29, 2022

(54) SMALL MOLECULE MODULATORS OF MHC-I

(71) Applicant: RetroVirox, Inc., San Diego, CA (US)

(72) Inventors: Juan LAMA, San Diego, CA (US); Robert I. HIGUCHI, Solana Beach, CA (US); Richard MARTIN, San Diego, CA (US); Maria Isabel CISNEROS, San Diego, CA (US)

(21) Appl. No.: 17/052,930

(22) PCT Filed: May 1, 2019
(86) PCT No.: PCT/US2019/030247

§ 371 Date: Nov. 4, 2020

Related U.S. Application Data
(60) Provisional application No. 62/667,341, filed on May 4, 2018.

(51) Int. Cl. C07D 417/14; C07D 417/12; C07D 405/12; C07D 405/14; C07D 277/60

(52) U.S. Cl. CPC C07D 417/14; C07D 417/12; C07D 277/60; C07D 405/14; C07D 405/12

(57) ABSTRACT

The invention disclosed herein are embodiments of compounds capable of treating a viral infection. For example, the compounds are capable of inhibiting viral downmodulation of major histocompatibility complex I (MHC-I), such as by acting as immunomodulators of the immune system to treat, cure or eradicate a viral infection (e.g., HIV infection). More particularly, the present disclosure relates to the use of a heteroaryl compound or salts or analogs thereof, in the treatment of patients infected with a virus. The disclosed compounds may be used alone or in combination with other pharmacologically active agents to treat, cure or eradicate the virus, particularly in patients with persistent, latent viral infection. In some embodiments, the disclosed compounds can be used alone or in combination with other pharmacologically active agents to promote reactivation of viral production in latent cells and eradication of such cells.

### CROSS-REFERENCE TO RELATED APPLICATIONS

This application claims priority to U.S. Application Ser. No. 62/667,341, filed on May 4, 2018. The disclosure of the prior application is considered part of the disclosure of this application, and is incorporated in its entirety into this application.

### STATEMENT OF GOVERNMENT SUPPORT

This invention was made with Government support under Grant Nos. 1R43AI100778 and 1R44AI100778, awarded by the National Institutes of Health. The Government has certain rights in the invention.

### BACKGROUND

### Technical Field

This disclosure relates to inhibitors of viral mediated downmodulation of major histocompatibility complex class I (MHC-I), and compositions comprising the same. More particularly, it concerns the use of heteroaryl compounds or salts or analogs thereof, in the treatment of patients infected with primate lentiviruses (e.g., human immunodeficiency viruses (HIV-1 and HIV-2) and simian immunodeficiency virus (SIV)).

### Background

A retrovirus designated human immunodeficiency virus (HIV), particularly the strains known as HIV type-1 (HIV-1) virus and type-2 (HIV-2) virus, is the etiological agent of acquired immunodeficiency syndrome (AIDS), a disease characterized by the destruction of the immune system, particularly of CD4 T-cells, with attendant susceptibility to opportunistic infections, and its precursor AIDS-related complex (“ARC”), a syndrome characterized by symptoms such as persistent generalized lymphadenopathy, fever, and weight loss. HIV was previously known as LAV, HTLV-III, or ARV.

Combination antiretroviral therapy (cART) has dramatically improved life expectancy and health of patients infected with HIV. Under controlled medical settings, over ninety percent of naïve patients achieve undetectable virus in plasma and normal CD4 counts. Nevertheless, when treatment interruption occurs, virus replication resumes in virtually all infected patients, highlighting the fact that cART does not cure HIV. The failure to eradicate HIV is due to the existence of latent reservoirs in which the viral genome remains integrated and transcriptionally inactive. These latent cells do not express viral RNA or viral proteins, and do not display any known markers that might reveal their presence. HIV latent cells cannot be recognized by the immune system and are resistant to the adaptive and innate responses [Siliciano, J. D., and R. F. Siliciano. 2006. The latent reservoir for HIV-1 in resting CD4+ T cells: a barrier to cure. Current opinion in HIV and AIDS 1:121-128]. Long-lived resting memory CD4 T cells, which have estimated half-lives of 44 years, hold the greatest HIV reservoir. Even with effective viral suppression by cART, elimination of this reservoir is estimated to take about 60 years [Siliciano, J. D., J. Kajdas, D. Finzi, T. C. Quinn, K. Chadwick, J. B. Margolick, C. Kovacs, S. J. Gange, and R. F. Siliciano. 2003. Long-term follow-up studies confirm the stability of the latent reservoir for HIV-1 in resting CD4+ T cells. Nature medicine 9:727-728]. As a first step to cure HIV, efforts have focused in the pharmacological reactivation of proviruses from latent reservoirs. Although major gaps still exist in this field, experimental evidence from ex-vivo and in-vivo studies has demonstrated the feasibility of HIV reactivation with the so-called latency-reversing agents (LRA). Notable LRAs include histone deacetylase inhibitors (HDACi), histone methyl transferase inhibitors (HMTi), NFkb and PTEF-B inducers, and protein kinase C (PKC) agonists [Mbonye, U., and J. Karn. 2014. Transcriptional control of HIV latency includes cellular signaling pathways and epigenetics. Virology 454-455:328-339]. Despite recent advances, when HIV reactivation is evaluated utilizing in vitro and ex-vivo latency models, autologous CTL cells fail to efficiently recognize and eliminate HIV reactivated cells [Shan, L., K. Deng, N. S. Shroff, C. M. Durand, S. A. Rabi, H. C. Yang, H. Zhang, J. B. Margolick, J. N. Blankson, and R. F. Siliciano. 2012. Stimulation of HIV-1-specific cytolytic T lymphocytes facilitates elimination of latent viral reservoir after virus reactivation. Immunity 36:491-501]. Reactivated cells also survive the virus-induced cytopathic effects (CPE). Interestingly, previous stimulation of CTL clones by different methods has proven successful at overcoming this problem, highlighting the feasibility of using approaches aimed at stimulating HIV-specific immune responses after LRA-mediated HIV reactivation. These two-step strategies are known as “shock and kill” therapies and it is likely that they will play a key role in future HIV cure approaches.

The failure of the immune response to eliminate reactivated cells may be explained by poor viral expression in reactivated cells, as compared to actively dividing producer cells. Diminished viral expression may make cells resistant to virus-induced CPE, and less prone to recognition by CTLs or specific antibodies that rely on surface presentation of viral antigens. In addition, suboptimal stimulation of the immune system in patients who achieve viral suppression with cART likely diminishes the magnitude and breadth of the HIV-specific CTL response. Importantly, the intrinsic ability of HIV to overcome the innate and adaptive immune responses may play an important role. Nef is known to downmodulate expression of the major histocompatibility complex type I (MHC-I) [Collins, D. R., and K. L. Collins. 2014. HIV-1 accessory proteins adapt cellular adaptors to facilitate immune evasion. PLoS pathogens 10:e1003851]. MIC-I is essential for the presentation of viral antigens to CD8-positive CTLs and the immune response mediated by CTLs plays a key role in the control of viral infections. It is widely accepted that by removing MHC-I, the HIV virus evades immune responses mediated by CTLs. Mathematical models have estimated that Nef activity may contribute to an average decrease of 82% in CTL killing efficiency [Wick, W. D., P. B. Gilbert, and O. O. Yang. 2009. Predicting the impact of blocking human immunodeficiency virus type 1 Nef in vivo. Journal of virology 83:2349-2356]. MHC-I downmodulation activity is highly conserved in alleles isolated from primary isolates. The significance of MHC-I downmodulation has been demonstrated with small animal models, infected macaques and in HIV-infected individuals. A point mutation near the C-terminus of SIVmac Nef that selectively disrupts the effect on MHC-I reverts in less than 4 weeks after macaques are challenged [Munch, J., N. Stolte, D. Fuchs, C. Stahl-Hennig, and F. Kirchhoff. 2001. Efficient class I major histocompatibility complex down-regulation by simian immunodeficiency virus Nef is associated with a strong selective advantage in infected rhesus macaques. Journal of virology 75:10532-10536]. The efficiency of CTL responses in infected patients seems to exert a selective pressure on the ability of Nef to downmodulate MHC-I, and this activity correlates positively with the breadth of the HIV-specific CTL response [Lewis, M. J., A. Balamurugan, A. Ohno, S. Kilpatrick, H. L. Ng, and O. O. Yang. 2008. Functional adaptation of Nef to the immune milieu of HIV-1 infection in vivo. Journal of immunology 180:4075-4081]. Further evidence of the importance of this function comes from unusually strong CTL responses observed in individuals infected with nef-defective HIV-1 strains [Dyer, W. B., G. S. Ogg, M. A. Demoitie, X. Jin, A. F. Geczy, S. L. Rowland-Jones, A. J. McMichael, D. F. Nixon, and J. S. Sullivan. 1999. Strong human immunodeficiency virus (HIV)-specific cytotoxic T-lymphocyte activity in Sydney Blood Bank Cohort patients infected with nef-defective HIV type 1. Journal of virology 73:436-443]. In summary, evidence suggests that small-molecule inhibitors of MIC-I downmodulation have the potential to overcome the ability of HIV-1 to escape immune surveillance, and could be used in “shock and kill” strategies to eradicate HIV infection.

### SUMMARY

The present disclosure provides methods and reagents, involving contacting a cell with an agent, such as thiazole, benzothiazole, pyridine or diazine derivatives, in a sufficient amount to reduce or specifically block viral-induced MIC-I downmodulation and reactivate or increase the CD8-mediated immune system response against the virus.

Some embodiments disclosed herein include inhibitors of viral mediated downmodulation of MIC-I containing either a thiazole, benzothiazole, pyridine or diazine core. Other embodiments disclosed herein include pharmaceutical compositions and methods of using these compounds.

One embodiment disclosed herein includes a compound having the structure of Formula I:

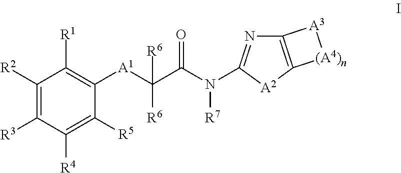

as well as well as enantiomers, diastereomers, tautomers, prodrugs, and pharmaceutically acceptable salts thereof.

In some embodiments of Formula (I):

R¹ and R² are taken together to form a ring which is selected from the group consisting of heteroaryl optionally substituted with 1-4 R⁸ and heterocyclyl optionally substituted with 1-4 R⁹;
with the proviso that

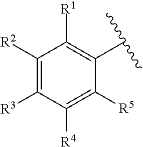

does not form

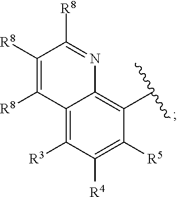

R³, R⁴, and R⁵ are independently selected from the group consisting of H, halide, unsubstituted —(C₁₋₆ alkyl), unsubstituted —(C₁₋₄ haloalkyl), —OR¹⁰, and —CN; each R⁶ is independently selected from the group consisting of H, unsubstituted —(C₁₋₆ alkyl), unsubstituted —(C₂₋₆ alkenyl), unsubstituted —(C₂₋₆ alkynyl), unsubstituted —(C₁-6 haloalkyl), —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein; R⁷ is selected from the group consisting of H, unsubstituted —(C₁₋₉ alkyl), unsubstituted —(C₃₋₉ alkenyl), unsubstituted —(C₃₋₉ alkynyl), unsubstituted —(C₁₋₉ haloalkyl), —(C₁₋₄ alkylene)C(═O)OR¹¹, —(C₁₋₄ alkylene)C(═O)N(R¹²)₂, —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein;
each R⁸ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR¹⁰, and —CN;
each R⁹ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
each R¹⁰ is independently selected from the group consisting of H, unsubstituted —(C₁₋₉ alkyl), unsubstituted —(C₃₋₉ alkenyl), unsubstituted —(C₃₋₉ alkynyl), and unsubstituted —(C₁₋₉ haloalkyl);
each R¹¹ is independently selected from the group consisting of H and unsubstituted —(C₁₋₉ alkyl);
each R¹² is independently selected from the group consisting of H and substituted or unsubstituted —(C₁₋₉ alkyl);
each R¹³ is independently selected from the group consisting of halide, unsubstituted —(C₁₋₉ alkyl), unsubstituted —(C₁₋₉ haloalkyl), and —OR¹⁰;
each R¹⁴ is independently selected from the group consisting of halide, unsubstituted —(C₁₋₉ alkyl), unsubstituted —(C₁₋₉ haloalkyl), and —OR¹⁰;
each R¹⁵ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁶ is independently selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
A¹ is selected from the group consisting of —C(R¹⁵)₂—, O, and S;
A² is selected from the group consisting of O and S;
A³ is selected from the group consisting of —C(R¹⁶)₂—, O and S;
each A⁴ is independently selected from the group consisting of —C(R¹⁶)₂—, O and S;
with the proviso that no more than two A³ and A⁴ are O or S and that two O or S are never adjacent;
n is 1, 2, 3, or 4;
each p independently is 0 or 1; and
with the proviso that Formula I is not a structure selected from the group consisting of:

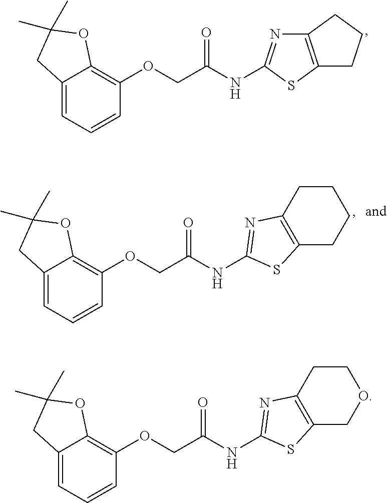 [SMILES: CC1(C)Cc2cccc(OCC(=O)Nc3nc4c(s3)CCC4)c2O1 ; CC1(C)Cc2cccc(OCC(=O)Nc3nc4c(s3)CCCC4)c2O1 ; CC1(C)Cc2cccc(OCC(=O)Nc3nc4c(s3)COCC4)c2O1]

One embodiment disclosed herein includes a compound having the structure of Formula II:

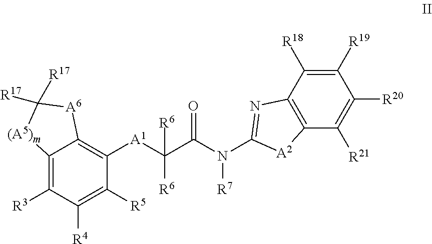

as well as enantiomers, diastereomers, tautomers, prodrugs, and

pharmaceutically acceptable salts thereof.

In some embodiments of Formula (II):

R³, R⁴, and R⁵ are independently selected from the group consisting of H, halide, unsubstituted —(C₁₋₆ alkyl), unsubstituted —(C₁₋₄ haloalkyl), —OR¹⁰, and —CN;
each R⁶ is independently selected from the group consisting of H, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
R⁷ is selected from the group consisting of H, unsubstituted —(C₁₋₉ alkyl), unsubstituted —(C₃₋₉ alkenyl), unsubstituted —(C₃₋₉ alkynyl), unsubstituted —(C₁₋₉ haloalkyl), —(C₁₋₄ alkylene)C(═O)OR¹¹, —(C₁₋₄ alkylene)C(═O)N(R¹²)₂, —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein;
each R¹⁰ is independently selected from the group consisting of H, unsubstituted —(C₁₋₉ alkyl), unsubstituted —(C₃₋₉ alkenyl), unsubstituted —(C₃₋₉ alkynyl), and unsubstituted —(C₁₋₉ haloalkyl);
R¹¹ is selected from the group consisting of H and unsubstituted —(C₁₋₉ alkyl);
each R¹² is independently selected from the group consisting of H and substituted or unsubstituted —(C₁₋₉ alkyl);
each R¹³ is independently selected from the group consisting of halide, unsubstituted —(C₁₋₉ alkyl), unsubstituted —(C₁₋₉ haloalkyl), and —OR¹⁰;
each R¹⁴ is independently selected from the group consisting of halide, unsubstituted —(C₁₋₉ alkyl), unsubstituted —(C₁₋₉ haloalkyl), and —OR¹⁰;
each R¹⁵ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁶ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁷ is independently selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R¹⁸ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR¹⁰, —(C₁₋₄ alkylene)ₚheteroaryl optionally substituted with 1-4 R²², —(C₁₋₄ alkylene)ₚaryl optionally substituted with 1-5 R²³, and —CN; wherein each —(C₁₋₄ alkylene) is, independently, optionally substituted with one or more substituents as defined herein;
R¹⁹ is selected from the group consisting of H, halide, unsubstituted —(C₁₋₉ alkyl), and unsubstituted —(C₁₋₆ haloalkyl);
R²⁰ is selected from the group consisting of H, halide, unsubstituted —(C₁₋₉ alkyl), and unsubstituted —(C₁₋₆ haloalkyl);
R²¹ is selected from the group consisting of H, halide, unsubstituted —(C₁₋₉ alkyl), and unsubstituted —(C₁₋₆ haloalkyl);
each R²² is independently selected from the group consisting of halide, unsubstituted —(C₁₋₉ alkyl), unsubstituted —(C₁₋₉ haloalkyl), and —CN;
each R²³ is independently selected from the group consisting of halide, unsubstituted —(C₁₋₉ alkyl), unsubstituted —(C₁₋₉ haloalkyl), and —CN;
A¹ is selected from the group consisting of —C(R¹⁵)₂—, O, and S;
A² is selected from the group consisting of O and S;
each A⁵ is independently selected from the group consisting of —C(R¹⁶)₂—, —N(R¹⁰)—, O, and S;
with the proviso that two A⁵ are not both N, O or S;
A⁶ is selected from the group consisting of —C(R¹⁶)₂—, —N(R¹⁰)—, O, and S;
with the proviso that A⁵ and A⁶ cannot both be C(R¹⁶)₂; m is 1 or 2;
each p independently is 0 or 1; and
with the proviso that Formula II is not a structure selected from the group consisting of:

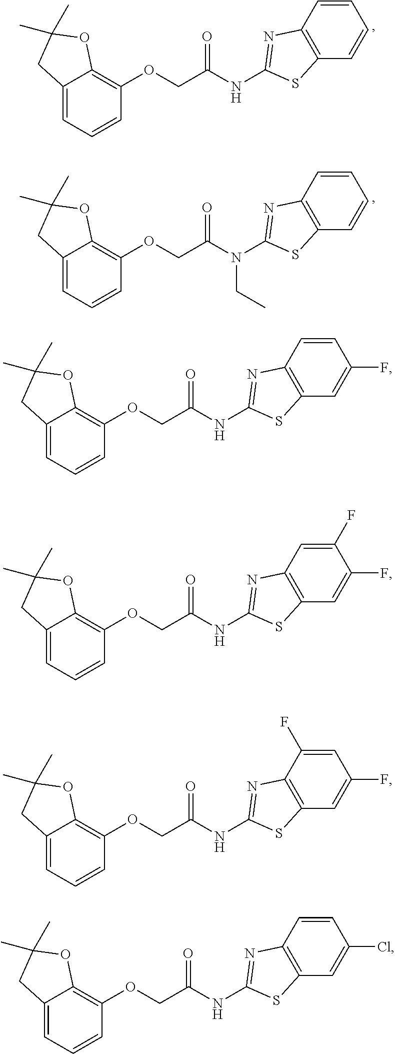

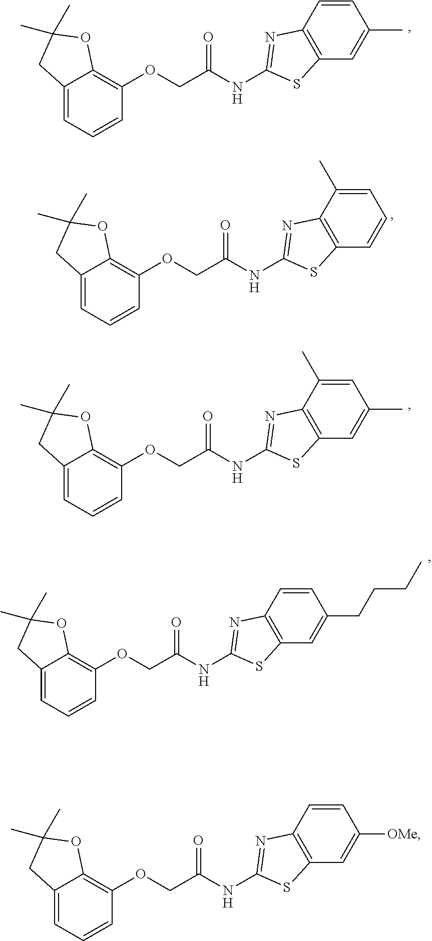

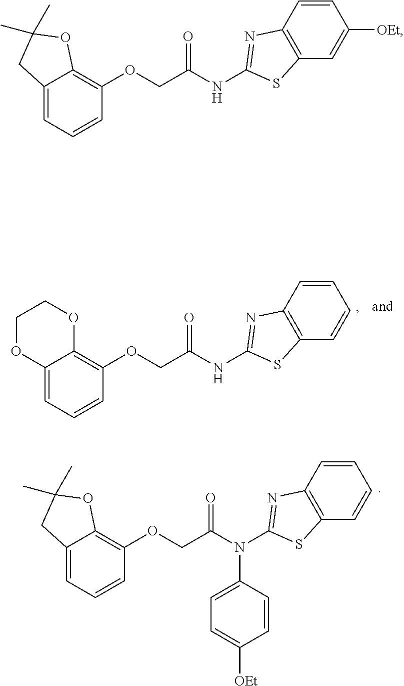 [SMILES: CCOc1ccc2nc(NC(=O)COc3cccc4c3OC(C)(C)C4)sc2c1 ; O=C(COc1cccc2c1OCCO2)Nc1nc2ccccc2s1 ; CCOc1ccc(N(C(=O)COc2cccc3c2OC(C)(C)C3)c2nc3ccccc3s2)cc1]

One embodiment disclosed herein includes a compound having the structure of Formula III:

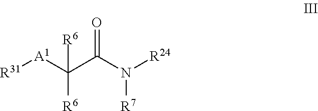

as well as enantiomers, diastereomers, tautomers, prodrugs, and pharmaceutically acceptable salts thereof.

In some embodiments of Formula (III):

R³, R⁴, and R⁵ are independently selected from the group consisting of H, halide, unsubstituted —(C₁₋₆ alkyl), unsubstituted —(C₁₋₄ haloalkyl), —OR¹⁰, and —CN;
each R⁶ is independently selected from the group consisting of H, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein;
R⁷ is selected from the group consisting of H, unsubstituted —(C₁₋₉ alkyl), unsubstituted —(C₃₋₉ alkenyl), unsubstituted —(C₃₋₉ alkynyl), unsubstituted —(C₁₋₉ haloalkyl), —(C₁₋₄ alkylene)C(═O)OR¹¹, —(C₁₋₄ alkylene)C(═O)N(R¹²)₂, —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein;
each R¹⁰ is independently selected from the group consisting of H, unsubstituted —(C₁₋₉ alkyl), unsubstituted —(C₃₋₉ alkenyl), unsubstituted —(C₃₋₉ alkynyl), and unsubstituted —(C₁₋₉ haloalkyl);
R¹¹ is selected from the group consisting of H and unsubstituted —(C₁₋₉ alkyl);
each R¹² is independently selected from the group consisting of H and substituted or unsubstituted —(C₁₋₉ alkyl);
each R¹³ is independently selected from the group consisting of halide, unsubstituted —(C₁₋₉ alkyl), unsubstituted —(C₁₋₉ haloalkyl), and —OR¹⁰;
each R¹⁴ is independently selected from the group consisting of halide, unsubstituted —(C₁₋₉ alkyl), unsubstituted —(C₁₋₉ haloalkyl), and —OR¹⁰;
each R¹⁵ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁶ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁷ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R²⁴ is selected from the group consisting of a 6-membered heteroaryl optionally substituted with 1-4 R²⁵,

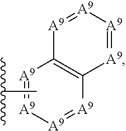

wherein 1-4 A⁹ are N; and

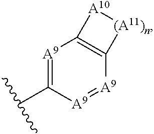

wherein 1-2 A⁹ are N;
each R²⁵ is independently selected from the group consisting of halide, —CN, —OR²⁸, unsubstituted —(C₁₋₆ alkyl), unsubstituted —(C₂₋₆ alkenyl), unsubstituted —(C₂₋₆ alkynyl), unsubstituted —(C₁₋₆ haloalkyl), and carbocyclyl optionally substituted with 1-10 R²⁹;
each R²⁶ is independently selected from the group consisting of H, halide, —CN, —OR²⁸, unsubstituted —(C₁₋₆ alkyl), unsubstituted —(C₂₋₆ alkenyl), unsubstituted —(C₂₋₆ alkynyl), unsubstituted —(C₁₋₆ haloalkyl), and carbocyclyl optionally substituted with 1-10 R²⁹;
each R²⁷ is independently selected from the group consisting of H, halide, —CN, —OR²⁸, unsubstituted —(C₁₋₆ alkyl), unsubstituted —(C₂₋₆ alkenyl), unsubstituted —(C₂₋₆ alkynyl), unsubstituted —(C₁₋₆ haloalkyl), and carbocyclyl optionally substituted with 1-10 R²⁹;
each R²⁸ is independently selected from the group consisting of unsubstituted —(C₁₋₉ alkyl), unsubstituted —(C₃₋₉ alkenyl), unsubstituted —(C₃₋₉ alkynyl), and unsubstituted —(C₁₋₉ haloalkyl);
each R²⁹ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
R³⁰ is selected from the group consisting of H and unsubstituted —(C₁₋₉ alkyl);
R³¹ is selected from the group consisting of

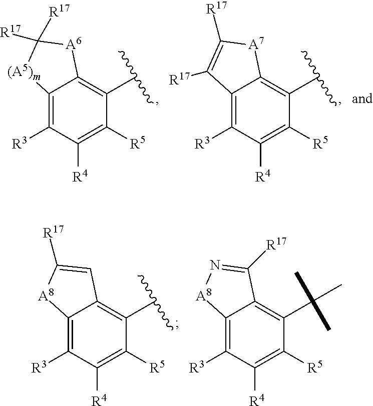

A¹ is selected from the group consisting of —C(R⁵)₂—, O, and S;
each A⁵ is independently selected from the group consisting of —C(R¹⁶)₂—, —N(R¹⁰)—, O, and S;
with the proviso that two A⁵ are not both N, O or S;
A⁶ is selected from the group consisting of —C(R¹⁶)₂—, —N(R¹⁰)—, O, and S;
with the proviso that A⁵ and A⁶ cannot both be C(R¹⁶)₂;
A⁷ is selected from the group consisting of —N(R³⁰)—, O and S;
A⁸ is selected from the group consisting of —NH—, O and S;
each A⁹ is independently selected from the group consisting of N and —C(R²⁶)—;
A¹⁰ is selected from the group consisting of —C(R²⁷)₂—, O, and S;
each A¹¹ is independently selected from the group consisting of —C(R²⁷)₂—, O, and S;
with the proviso that no more than two A¹⁰ or A¹¹ are O or S and that two O or S are never adjacent;
n is 1, 2, 3, or 4;
m is 1 or 2;

each p independently is 0 or 1; and

with the proviso that Formula III is not a structure selected from the group consisting of:

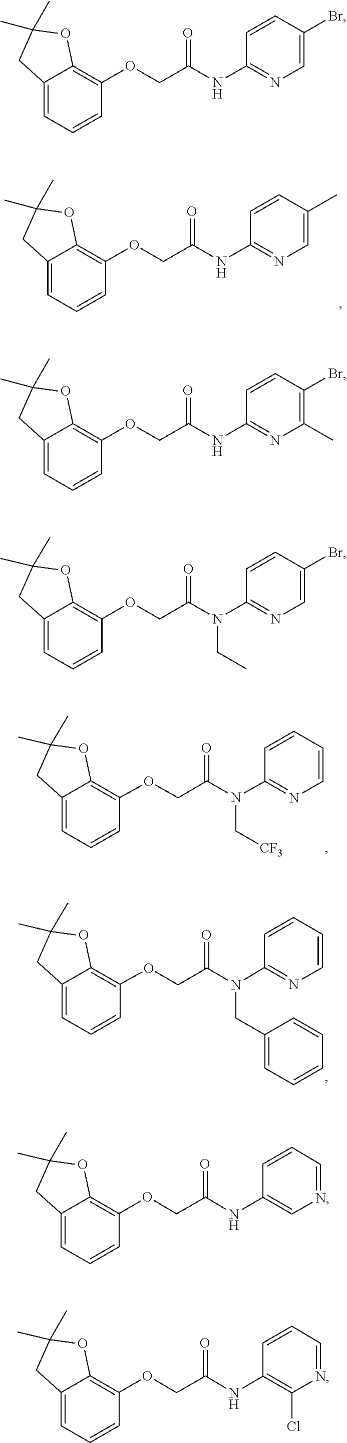

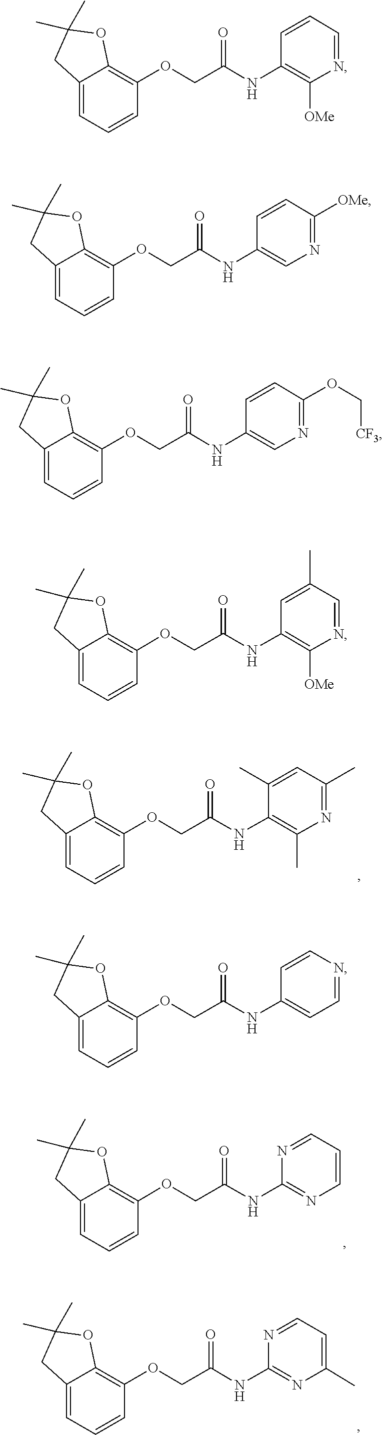

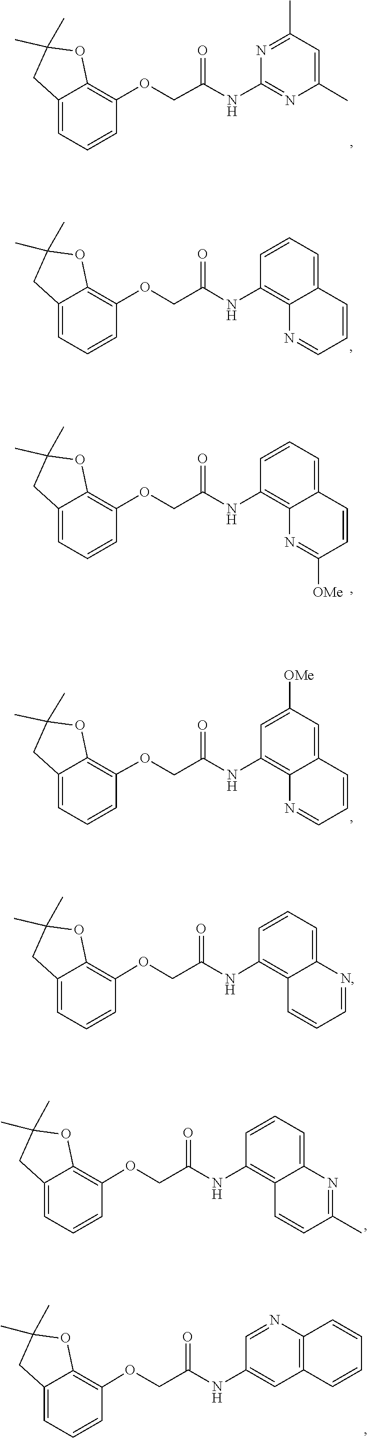

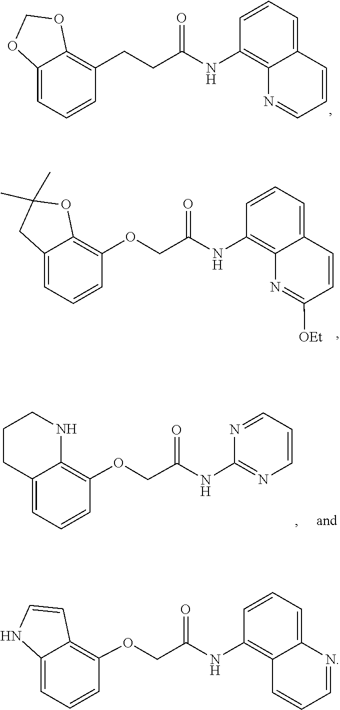 [SMILES: O=C(CCc1cccc2c1OCO2)Nc1cccc2cccnc12 ; CCOc1ccc2cccc(NC(=O)COc3cccc4c3OC(C)(C)C4)c2n1 ; O=C(COc1cccc2c1NCCC2)Nc1ncccn1 ; O=C(COc1cccc2[nH]ccc12)Nc1cccc2ncccc12]

One embodiment disclosed herein includes a compound having the structure of Formula IV:

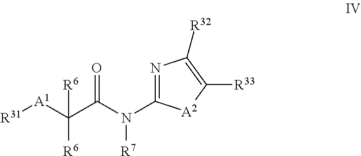

as well as enantiomers, diastereomers, tautomers, prodrugs, and pharmaceutically acceptable salts thereof.

In some embodiments of Formula (IV):

R³, R⁴, and R⁵ are independently selected from the group consisting of H, halide, unsubstituted —(C₁₋₆ alkyl), unsubstituted —(C₁₋₄ haloalkyl), —OR¹⁰, and —CN;
each R⁶ is independently selected from the group consisting of H, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
R⁷ is selected from the group consisting of H, unsubstituted —(C₁₋₉ alkyl), unsubstituted —(C₃₋₉ alkenyl), unsubstituted —(C₃₋₉ alkynyl), unsubstituted —(C₁₋₉ haloalkyl), —(C₁₋₄ alkylene)C(═O)OR¹¹, —(C₁₋₄ alkylene)C(═O)N(R¹²)₂, —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein;
each R¹⁰ is independently selected from the group consisting of H, unsubstituted —(C₁₋₉ alkyl), unsubstituted —(C₃₋₉ alkenyl), unsubstituted —(C₃₋₉ alkynyl), and unsubstituted —(C₁₋₉ haloalkyl);
R¹¹ is selected from the group consisting of H and unsubstituted —(C₁₋₉ alkyl);
each R¹² is independently selected from the group consisting of H and substituted or unsubstituted —(C₁₋₉ alkyl);
each R¹³ is independently selected from the group consisting of halide, unsubstituted —(C₁₋₉ alkyl), unsubstituted —(C₁₋₉ haloalkyl), and —OR¹⁰;
each R¹⁴ is independently selected from the group consisting of halide, unsubstituted —(C₁₋₉ alkyl), unsubstituted —(C₁₋₉ haloalkyl), and —OR¹⁰;
each R¹⁵ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁶ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁷ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R³⁰ is selected from the group consisting of H and unsubstituted —(C₁₋₉ alkyl);
R³¹ is selected from the group consisting of

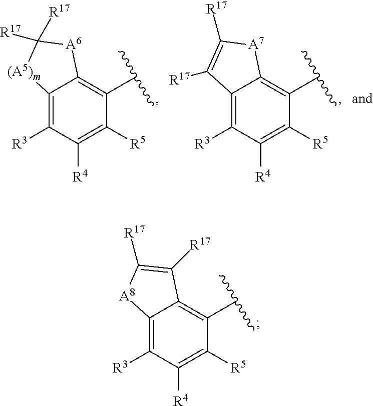

R³² is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -carbocyclyl optionally substituted with 1-10 R³⁴, —C(═O)R³⁵, —C(═O)NHR³⁶, and —CN;
R³³ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -carbocyclyl optionally substituted with 1-10 R³⁷, —C(═O)R³⁸, —C(═O)NHR³⁹, and —CN;
each R³⁴ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
R³⁵ is -heterocyclyl optionally substituted with 1-10 R⁴⁰;
R³⁶ is selected from the group consisting of —(C₁₋₄ alkylene)heterocyclyl optionally substituted with 1-10 R⁴¹ and —(C₁₋₄ alkylene)heteroaryl optionally substituted with 1-10 R⁴²; wherein each —(C₁₋₄ alkylene) is, independently, optionally substituted with one or more substituents as defined herein;
each R³⁷ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
R³⁸ is -heterocyclyl optionally substituted with 1-10 R⁴⁴;
R³⁹ is selected from the group consisting of —(C₁₋₄ alkylene)heterocyclyl optionally substituted with 1-10 R⁴⁵ and —(C₁₋₄ alkylene)heteroaryl optionally substituted with 1-10 R⁴⁶;
each R⁴¹ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(C₁₋₄ alkylene)ₚOR⁴³; wherein —(C₁₋₄ alkylene) is optionally substituted with one or more substituents as defined herein;
each R⁴¹ is independently selected from the group consisting of halide, unsubstituted —(C₁₋₉ alkyl), and

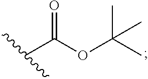 [SMILES: *C(=O)OC(C)(C)C]

each R⁴² is independently selected from the group consisting of halide and unsubstituted —(C₁₋₉ alkyl);
each R⁴³ is independently selected from the group consisting of H and unsubstituted —(C₁₋₉ alkyl);
each R⁴⁴ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(C₁₋₄ alkylene)ₚOR⁴⁷; wherein —(C₁₋₄ alkylene) is optionally substituted with one or more substituents as defined herein;
each R⁴⁵ is independently selected from the group consisting of halide, unsubstituted —(C₁₋₉ alkyl), and

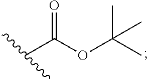 [SMILES: *C(=O)OC(C)(C)C]

each R⁴⁶ is independently selected from the group consisting of halide and unsubstituted —(C₁₋₉ alkyl);
each R⁴⁷ is independently selected from the group consisting of H and unsubstituted —(C₁₋₉ alkyl);
A¹ is selected from the group consisting of —C(R¹⁵)₂—, O, and S;
A² is selected from the group consisting of O and S;
each A⁵ is independently selected from the group consisting of —C(R¹⁶)₂—, —N(R¹⁰)—, O, and S;
with the proviso that two A⁵ are not both N, O or S;
A⁶ is selected from the group consisting of —C(R¹⁶)₂—, —N(R¹⁰)—, O, and S;
with the proviso that A⁵ and A⁶ cannot both be C(R¹⁶)₂;
A⁷ is selected from the group consisting of —N(R³⁰)—, O and S;
A⁸ is selected from the group consisting of —N(R³⁰)—, O and S;

m is 1 or 2;

each p independently is 0 or 1; and
with the proviso that Formula IV is not a structure selected from the group consisting of:

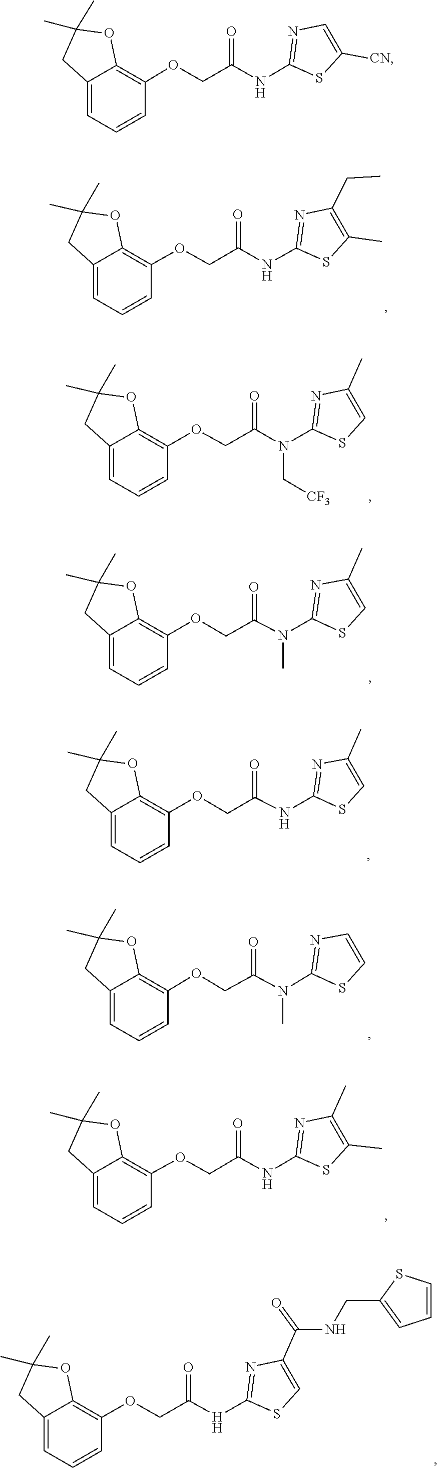

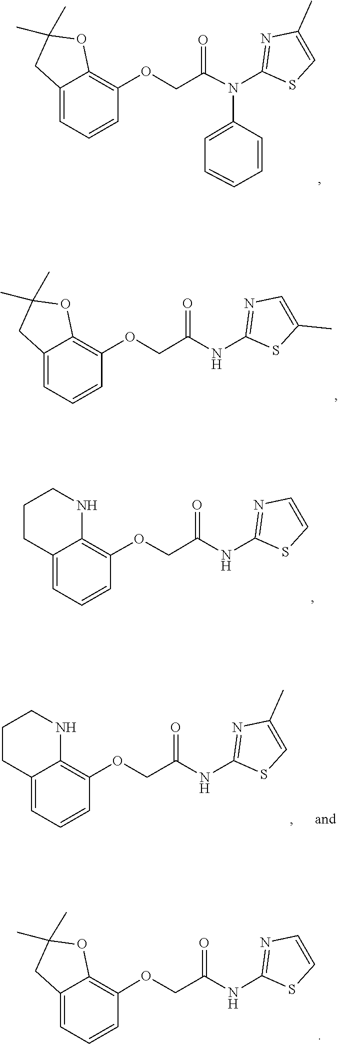

One embodiment disclosed herein includes a compound having the structure of Formula V:

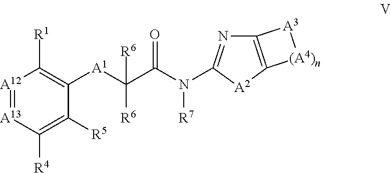

as well as enantiomers, diastereomers, tautomers, prodrugs, and pharmaceutically acceptable salts thereof.

In some embodiments of Formula (V):

R¹ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR⁴⁸, -carbocyclyl optionally substituted with 1-4 R⁴⁹, and —CN;
R² is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR⁴⁸, -carbocyclyl optionally substituted with 1-4 R⁴⁹, and —CN;
R³ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR⁴⁸, -carbocyclyl optionally substituted with 1-4 R⁴⁹, and —CN;
R⁴ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR⁴⁸, -carbocyclyl optionally substituted with 1-4 R⁴⁹, and —CN;
R⁵ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR⁴⁸, -carbocyclyl optionally substituted with 1-4 R⁴⁹, and —CN;
with the proviso that when A¹² is —C(R²)— and A¹³ is —C(R³)—, then R¹, R², R³, R⁴, and R⁵ are not all H;
with the proviso that when A¹² is N, then R¹, R³, R⁴, and R⁵ are not all H;
each R⁶ is independently selected from the group consisting of H, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
R⁷ is selected from the group consisting of unsubstituted —(C₁₋₉ alkyl), unsubstituted —(C₃₋₉ alkenyl), unsubstituted —(C₃₋₉ alkynyl), unsubstituted —(C₁₋₉ haloalkyl), —(C₁₋₄ alkylene)C(═O)OR¹¹, —(C₁₋₄ alkylene)C(═O)N(R¹²)₂, —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein;
each R¹⁰ is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
R¹¹ is selected from the group consisting of H and unsubstituted —(C₁₋₉ alkyl);
each R¹² is independently selected from the group consisting of H and substituted or unsubstituted —(C₁₋₉ alkyl);
each R¹³ is independently selected from the group consisting of halide, unsubstituted —(C₁₋₉ alkyl), unsubstituted —(C₁₋₉ haloalkyl), and —OR¹⁰;
each R¹⁴ is independently selected from the group consisting of unsubstituted —(C₁₋₉ alkyl), unsubstituted —(C₁₋₉ haloalkyl), and —OR¹⁰;
each R¹⁵ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁶ is independently selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
each R⁴⁸ is independently selected from the group consisting of unsubstituted —(C₁₋₉ alkyl), unsubstituted —(C₃₋₉ alkenyl), unsubstituted —(C₃₋₉ alkynyl), and unsubstituted —(C₁₋₉ haloalkyl);
each R⁴⁹ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
A¹ is selected from the group consisting of —C(R¹⁵)₂—, O, and S;
A² is selected from the group consisting of O and S;
A³ is selected from the group consisting of —C(R¹⁶)₂—, O and S;
each A⁴ is independently selected from the group consisting of —C(R¹⁶)₂—, O and S;
with the proviso that no more than two of A³ and/or A⁴ are O or S and that two O or S are never adjacent;
A¹² is selected from the group consisting of —C(R²)— and N;
A¹³ is selected from the group consisting of —C(R³)— and N;
n is 1 to 4;
each p independently is 0 to 1; and
with the proviso that Formula V is not a structure selected from the group consisting of:

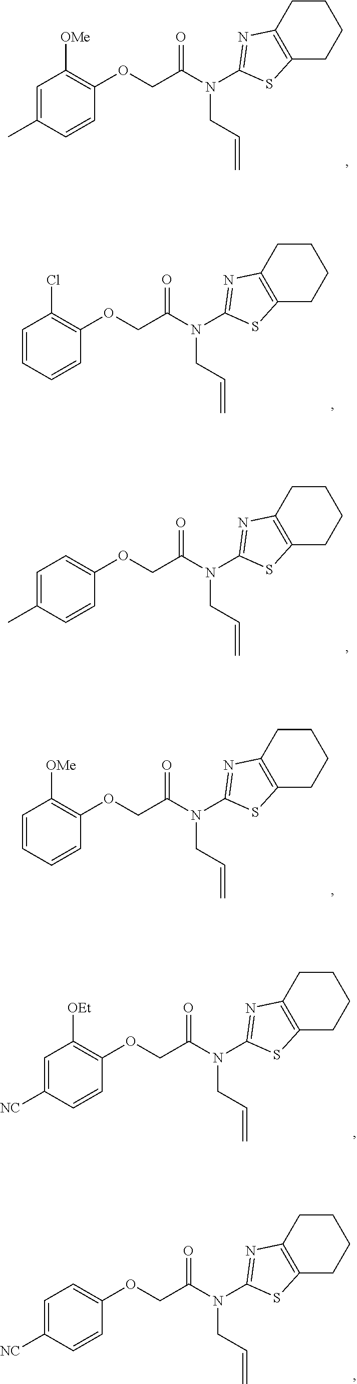 [SMILES: C=CCN(C(=O)COc1ccc(C)cc1OC)c1nc2c(s1)CCCC2 ; C=CCN(C(=O)COc1ccccc1Cl)c1nc2c(s1)CCCC2 ; C=CCN(C(=O)COc1ccc(C)cc1)c1nc2c(s1)CCCC2 ; C=CCN(C(=O)COc1ccccc1OC)c1nc2c(s1)CCCC2 ; C=CCN(C(=O)COc1ccc(C#N)cc1OCC)c1nc2c(s1)CCCC2 ; C=CCN(C(=O)COc1ccc(C#N)cc1)c1nc2c(s1)CCCC2]

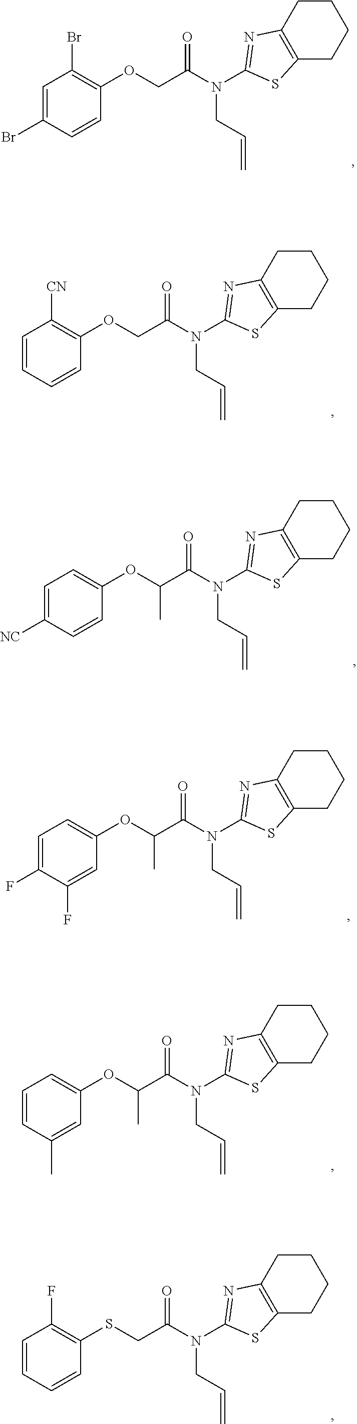 [SMILES: C=CCN(C(=O)COc1ccc(Br)cc1Br)c1nc2c(s1)CCCC2 ; C=CCN(C(=O)COc1ccccc1C#N)c1nc2c(s1)CCCC2 ; C=CCN(C(=O)C(C)Oc1ccc(C#N)cc1)c1nc2c(s1)CCCC2 ; C=CCN(C(=O)C(C)Oc1ccc(F)c(F)c1)c1nc2c(s1)CCCC2 ; C=CCN(C(=O)C(C)Oc1cccc(C)c1)c1nc2c(s1)CCCC2 ; C=CCN(C(=O)CSc1ccccc1F)c1nc2c(s1)CCCC2]

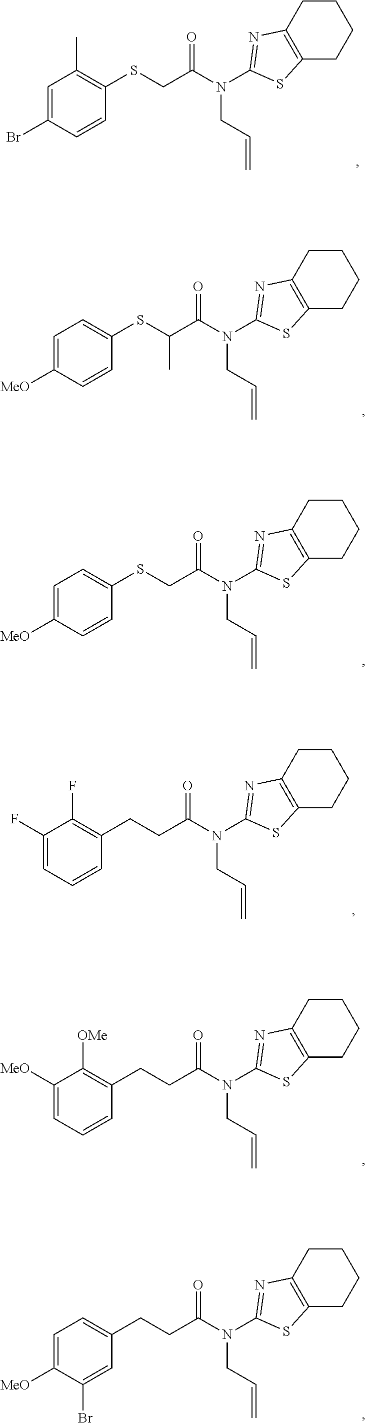 [SMILES: C=CCN(C(=O)CSc1ccc(Br)cc1C)c1nc2c(s1)CCCC2 ; C=CCN(C(=O)C(C)Sc1ccc(OC)cc1)c1nc2c(s1)CCCC2 ; C=CCN(C(=O)CSc1ccc(OC)cc1)c1nc2c(s1)CCCC2 ; C=CCN(C(=O)CCc1cccc(F)c1F)c1nc2c(s1)CCCC2 ; C=CCN(C(=O)CCc1cccc(OC)c1OC)c1nc2c(s1)CCCC2 ; C=CCN(C(=O)CCc1ccc(OC)c(Br)c1)c1nc2c(s1)CCCC2]

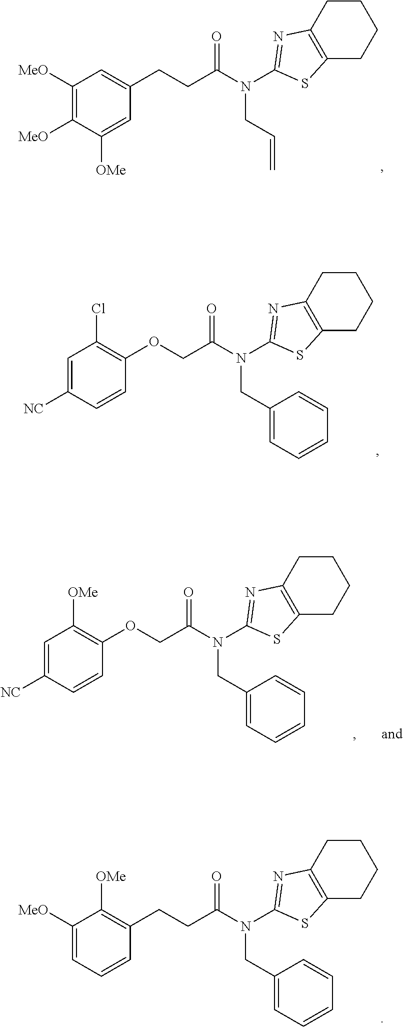 [SMILES: C=CCN(C(=O)CCc1cc(OC)c(OC)c(OC)c1)c1nc2c(s1)CCCC2 ; N#Cc1ccc(OCC(=O)N(Cc2ccccc2)c2nc3c(s2)CCCC3)c(Cl)c1 ; COc1cc(C#N)ccc1OCC(=O)N(Cc1ccccc1)c1nc2c(s1)CCCC2 ; COc1cccc(CCC(=O)N(Cc2ccccc2)c2nc3c(s2)CCCC3)c1OC]

One embodiment disclosed herein includes a compound having the structure of Formula VI:

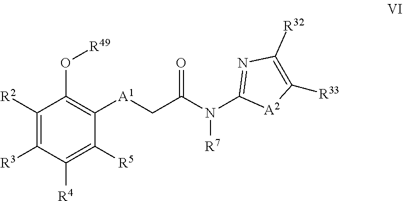

as well as enantiomers, diastereomers, tautomers, prodrugs, and pharmaceutically acceptable salts thereof.

In some embodiments of Formula (VI):

R² is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R³ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R⁴ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R⁵ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R⁷ is selected from the group consisting of H, unsubstituted —(C₁₋₉ alkyl), unsubstituted —(C₃₋₉ alkenyl), unsubstituted —(C₃₋₉ alkynyl), unsubstituted —(C₁₋₉ haloalkyl), —(C₁₋₄ alkylene)C(═O)OR¹¹, —(C₁₋₄ alkylene)C(═O)N(R¹²)₂, —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein;
each R¹⁰ is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
R¹¹ is selected from the group consisting of H and unsubstituted —(C₁₋₉ alkyl);
each R¹² is independently selected from the group consisting of H and substituted or unsubstituted —(C₁₋₉ alkyl);
each R¹³ is independently selected from the group consisting of halide, unsubstituted —(C₁₋₉ alkyl), unsubstituted —(C₁₋₉ haloalkyl), and —OR¹⁰;
each R¹⁴ is independently selected from the group consisting of halide, unsubstituted —(C₁₋₉ alkyl), unsubstituted —(C₁₋₉ haloalkyl), and —OR¹⁰;
each R¹⁵ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
R³² is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ fluoroalkyl), and —CN;
R³³ is selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R⁴⁹ is selected from the group consisting of unsubstituted —(C₃₋₉ alkyl), unsubstituted —(C₃₋₉ alkenyl), unsubstituted —(C₃₋₉ alkynyl), unsubstituted —(C₃₋₉ haloalkyl), —(C₁₋₄ alkylene)ₚheteroaryl optionally substituted with 1-4 R⁵⁰, and —(C₁₋₄ alkylene)ₚaryl optionally substituted with 1-5 R⁵¹, and —(C₁₋₄ alkylene)ₚcarbocyclyl optionally substituted with 1-10 R⁵²; wherein each —(C₁₋₄ alkylene) is, independently, optionally substituted with one or more substituents as defined herein;
each R⁵⁰ is independently selected from the group consisting of halide, unsubstituted —(C₁₋₉ alkyl), unsubstituted —(C₁₋₉ haloalkyl), and —OR¹⁰;
each R⁵¹ is independently selected from the group consisting of halide, unsubstituted —(C₁₋₉ alkyl), unsubstituted —(C₁₋₉ haloalkyl), and —OR¹⁰;
each R⁵² is independently selected from the group consisting of halide, unsubstituted —(C₁₋₉ alkyl), and unsubstituted —(C₁₋₉ haloalkyl);
A¹ is selected from the group consisting of —C(R¹⁵)₂—, O, and S;
A² is selected from the group consisting of O and S;
each p independently is 0 or 1; and
with the proviso that Formula VI is not:

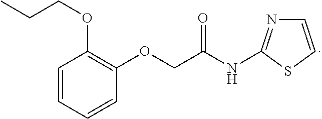 [SMILES: CCCOc1ccccc1OCC(=O)Nc1nccs1]

Some embodiments include stereoisomers and pharmaceutically acceptable salts of a compound of Formulas I, II, III, IV, V, and VI. Some embodiments include pharmaceutically acceptable salts of a compound of Formula (I).

Some embodiments include pro-drugs of a compound of Formulas I, II, III, IV, V, and VI.

Some embodiments of the present disclosure include pharmaceutical compositions comprising a compound of Formulas I, II, III, IV, V, and VI and a pharmaceutically acceptable carrier, diluent, or excipient.

Also provided herein are methods of treating a viral infection in a patient, the method comprising administering to the patient an effective amount of a compound according to Formulas I, II, III, IV, V, and VI. In some embodiments, the methods provided herein further comprise administration of a latency-reversing agent (LRA). The LRA can be administered before, after, or concurrently with the compound according to Formulas I, II, III, IV, V, and VI. In some embodiments, the LRA is administered before administration of the compound according to Formulas I, II, III, IV, V, and VI. In some embodiments, the methods provided herein further comprise administration of a second antiviral agent. For example, an antiviral agent as provided herein. The second antiviral agent can be administered before, after, or concurrently with the compound according to Formulas I, II, III, IV, V, and VI. In some embodiments, the second antiviral agent is administered after administration of the compound according to Formulas I, II, III, IV, V, and VI.

Other embodiments disclosed herein include methods of inhibiting or reducing viral mediated downmodulation of MHC-I by administering to a patient (e.g., a patient infected by a primate lentivirus, such as HIV-1, HIV-2 and SIV), an effective amount of a compound according to Formulas I, II, III, IV, V, and VI. For example, the compounds and compositions provided herein can be used to treat HIV-1, HIV-2 and SIV.

Some embodiments of the present disclosure include methods to prepare compounds of Formulas I, II, III, IV, V, and VI.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-1F are flow cytometry dot plots of SupT1 cells in which MHC-I is downmodulated by HIV-1 infection. SupT1 cells were either uninfected (FIG. 1A), infected with wild-type (‘WT”) HIV-1 reporter virus (NL4.3-GFP)(in FIGS. 1B, 1D, 1E, and 1F), or infected with the Nef-defective version NL4.3-ΔNef-GFP (ΔNef) in FIG. 1C. Y-axis represents the levels of MHC-I surface expression (APC signal after staining with anti-HLA-A2 antibody). X-axis indicates the levels of GFP expression in infected cells (FITC fluorescence signal). The different gates used to analyze MHC-I downmodulation are shown. The details are explained in the text.

FIGS. 2A-2D are flow cytometry dot plots of MHC-I downmodulation in SupT1 cells infected with HIV-1 in the presence of compound #40. SupT1 cells were either left uninfected (FIG. 2C), infected with wild-type HIV-1 reporter (NL4.3-GFP)(FIGS. 2A and 2B), or infected with a Nef-defective version NL4.3-ΔNef-GFP (ΔNef) in FIG. 2C. FIG. 2B shows cells incubated with compound #40 at 100 nM, which is compared to vehicle alone in FIG. 2A. Y-axis represents the levels of MHC-I surface expression X-axis indicates the levels of GFP expression in infected cells. The details are explained in the text.

FIG. 2E represents the level of MHC-I downmodulation achieved in dot plots 2A (vehicle alone), 2B (compound #40) and 2D (nef-defective virus). MHC-I downmodulation with wild-type HIV-1 in the presence of compound #40 (100 nM) is monitored by the parameters “R1”, “R4”, and “DM” and compared to the downmodulation observed in wild-type infected cells with vehicle alone (WT DMSO) or in ΔNef infected cells with vehicle alone (ΔNef−DMSO).

FIG. 2F and FIG. 2G compare either the extent of HIV-infection (FIG. 2F) as measured by GFP fluorescence, or the cell viability, as estimated by FS/SS flow cytometry analyses (FIG. 2G) in HIV-1 wild-type infected cells with vehicle alone (“WT”), in the presence of compound #40 at 100 nM concentration (“WT-40”), or in ΔNef infected cells with vehicle alone (“ΔNef”).

FIG. 3A, FIG. 3B and FIG. 3C represent dose-dependent inhibition curves of MHC-I downmodulation in SupT1 cells infected with HIV-1 reporter virus (NL4.3-GFP). The extent of MHC-I downmodulation is monitored by estimating “R1” (FIG. 3A), “R4” (FIG. 3B) or “DM” values (FIG. 3C). MHC-I downmodulation observed in cells treated with different concentrations of compounds #40, #53, #54, #56 or #100 is shown as the percentage of the activity observed in cells treated with 0.1% DMSO. EC₅₀ values (nM concentration reducing MHC-I downmodulation by 50%) are shown in each figure for each compound. FIG. 3A also includes CC₅₀ concentrations in micromolar values (reducing cell viability by 50%) and the selectivity index (“SI”) (CC₅₀ divided by EC₅₀) for each compound.

FIG. 4 represents the levels of expression of surface MHC-I in uninfected SupT1 cells (GFP-negative) incubated with vehicle alone (0.1% DMSO) or with compounds #40, #53, #54, #56 or #100 at different concentrations. Values are shown as the percentage of the MHC-I expression observed in cells treated with 0.1% DMSO alone. All samples were incubated with HIV-1 reporter virus (NL4.3-GFP) except those shown as ΔNef, which are shown for reference and were treated with nef-defective virus.

FIG. 5 shows the effect of compounds #40, #53, #54, #56 or #100 in infection of HeLa reporter cells with HIV-1. LAI strain was used to infect HeLa-CD4-LTR-B-gal reporter cells in the presence of compounds. The extent of HIV infection is measured by monitoring after 48 h of infection the β-galactosidase activity with a commercial kit (Galacto-Star) and compared with cells treated with vehicle alone. The table in FIG. 5 displays EC₅₀ concentrations (μM) for each compound, and also CC₅₀ concentrations in uninfected cells (reducing cell viability by 50% as estimated with the XTT method) and selectivity indices (SI) obtained by dividing CC₅₀ by EC₅₀ values.

FIG. 6A and FIG. 6B. compares the ability of compounds 40, 53, 56, 94, 143, or 195 to enhance MHC-I surface expression in uninfected SupT1 cells (FIG. 6A), with the ability of the same compounds to inhibit surface MHC-I downmodulation in HIV-1 infected SupT1 cells (FIG. 6B). EC₅₀ values for both assays are shown in the tables below in nanomolar concentrations.

### DETAILED DESCRIPTION

Embodiments described herein relate to compositions and methods for inhibiting viral mediated downmodulation of MHC-I, and to compositions and methods useful for the treatment or eradication of a virus in a subject.

Some embodiments provided herein relate to a method for treating a disease including, but not limited to, human immunodeficiency viruses (HIV-1 and HIV-2) and simian immunodeficiency virus (SIV).

### Definitions

Unless defined otherwise, all technical and scientific terms used herein have the same meaning as is commonly understood by one of ordinary skill in the art to which this disclosure belongs. All patents, applications, published applications, and other publications are incorporated by reference in their entirety. In the event that there is a plurality of definitions for a term herein, those in this section prevail unless stated otherwise.

As used herein, “alkyl” means a branched, or straight chain chemical group containing only carbon and hydrogen, such as methyl, ethyl, n-propyl, iso-propyl, n-butyl, iso-butyl, sec-butyl, tert-butyl, n-pentyl, iso-pentyl, sec-pentyl and neo-pentyl. Alkyl groups can either be unsubstituted or substituted with one or more substituents. In some embodiments, alkyl groups include 1 to 9 carbon atoms (for example, 1 to 6 carbon atoms, 1 to 4 carbon atoms, or 1 to 2 carbon atoms).

As used herein, “alkenyl” means a straight or branched chain chemical group containing only carbon and hydrogen and containing at least one carbon-carbon double bond, such as ethenyl, 1-propenyl, 2-propenyl, 2-methyl-1-propenyl, 1-butenyl, 2-butenyl, and the like. In various embodiments, alkenyl groups can either be unsubstituted or substituted with one or more substituents. Typically, alkenyl groups will comprise 2 to 9 carbon atoms (for example, 2 to 6 carbon atoms, 2 to 4 carbon atoms, or 2 carbon atoms).

As used herein, “alkynyl” means a straight or branched chain chemical group containing only carbon and hydrogen and containing at least one carbon-carbon triple bond, such as ethynyl, 1-propynyl, 1-butynyl, 2-butynyl, and the like. In various embodiments, alkynyl groups can either be unsubstituted or substituted with one or more substituents. Typically, alkynyl groups will comprise 2 to 9 carbon atoms (for example, 2 to 6 carbon atoms, 2 to 4 carbon atoms, or 2 carbon atoms).

As used herein, “alkylene” means a bivalent branched, or straight chain chemical group containing only carbon and hydrogen, such as methylene, ethylene, n-propylene, iso-propylene, n-butylene, iso-butylene, sec-butylene, tert-butylene, n-pentylene, iso-pentylene, sec-pentylene and neo-pentylene. Alkylene groups can either be unsubstituted or substituted with one or more substituents. Alkylene groups can be saturated or unsaturated (e.g., containing —C═C— or —C≡C— subunits), at one or several positions. In some embodiments, alkylene groups include 1 to 9 carbon atoms (for example, 1 to 6 carbon atoms, 1 to 4 carbon atoms, or 1 to 2 carbon atoms).

As used herein, “alkenylene” means a bivalent branched, or straight chain chemical group containing only carbon and hydrogen and containing at least one carbon-carbon double bond, such as ethenylene, 1-propenylene, 2-propenylene, 2-methyl-1-propenylene, 1-butenylene, 2-butenylene, and the like. In various embodiments, alkenylene groups can either be unsubstituted or substituted with one or more substituents. Typically, alkenylene groups will comprise 2 to 9 carbon atoms (for example, 2 to 6 carbon atoms, 2 to 4 carbon atoms, or 2 carbon atoms).

As used herein, “alkynylene” means a bivalent branched, or straight chain chemical group containing only carbon and hydrogen and containing at least one carbon-carbon triple bond, such as ethynylene, 1-propynylene, 1-butynylene, 2-butynylene, and the like. In various embodiments, alkynylene groups can either be unsubstituted or substituted with one or more substituents. Typically, alkynylene groups will comprise 2 to 9 carbon atoms (for example, 2 to 6 carbon atoms, 2 to 4 carbon atoms, or 2 carbon atoms).

As used herein, “alkoxy” means an alkyl-O group in which the alkyl group is as described herein. Exemplary alkoxy groups include methoxy, ethoxy, n-propoxy, i-propoxy, n-butoxy, s-butoxy, t-butoxy, pentoxy, hexoxy and heptoxy, and also the linear or branched positional isomers thereof.

As used herein, “haloalkoxy” means an haloalkyl-O group in which the haloalkyl group is as described herein. Exemplary haloalkoxy groups include fluoromethoxy, difluoromethoxy, trifluoromethoxy, and also the linear or branched positional isomers thereof.

As used herein, “carbocyclyl” means a cyclic ring system containing only carbon atoms in the ring system backbone, such as cyclopropyl, cyclobutyl, cyclopentyl, cyclohexyl, and cyclohexenyl. Carbocyclyls may include multiple fused rings. Carbocyclyls may have any degree of saturation provided that at least one ring in the ring system is not aromatic. Carbocyclyl groups can either be unsubstituted or substituted with one or more substituents. In some embodiments, carbocyclyl groups include 3 to 10 carbon atoms, for example, 3 to 6 carbon atoms.

As used herein, “aryl” means a mono-, bi-, tri- or polycyclic group with only carbon atoms present in the ring backbone having 5 to 14 ring atoms, alternatively 5, 6, 9, or 10 ring atoms; and having 6, 10, or 14 pi electrons shared in a cyclic array; wherein at least one ring in the system is aromatic. Aryl groups can either be unsubstituted or substituted with one or more substituents. Examples of aryl include phenyl, naphthyl, tetrahydronaphthyl, 2,3-dihydro-1H-indenyl, and others. In some embodiments, the aryl is phenyl.

As used herein, “arylalkylene” means an aryl-alkylene-group in which the aryl and alkylene moieties are as previously described. In some embodiments, arylalkylene groups contain a C₁₋₄alkylene moiety. Exemplary arylalkylene groups include benzyl and 2-phenethyl.

As used herein, the term “heteroaryl” means a mono-, bi-, tri- or polycyclic group having 5 to 14 ring atoms, alternatively 5, 6, 9, or 10 ring atoms; and having 6, 10, or 14 pi electrons shared in a cyclic array; wherein at least one ring in the system is aromatic, and at least one ring in the system contains one or more heteroatoms independently selected from the group consisting of N, O, and S. Heteroaryl groups can either be unsubstituted or substituted with one or more substituents. Examples of heteroaryl include thienyl, pyridinyl, furyl, oxazolyl, oxadiazolyl, pyrrolyl, imidazolyl, triazolyl, thiodiazolyl, pyrazolyl, isoxazolyl, thiadiazolyl, pyranyl, pyrazinyl, pyrimidinyl, pyridazinyl, triazinyl, thiazolyl benzothienyl, benzoxadiazolyl, benzofuranyl, benzimidazolyl, benzotriazolyl, cinnolinyl, indazolyl, indolyl, isoquinolinyl, isothiazolyl, naphthyridinyl, purinyl, thienopyridinyl, pyrido[2,3-d]pyrimidinyl, pyrrolo[2,3-b]pyridinyl, quinazolinyl, quinolinyl, thieno[2,3-c]pyridinyl, pyrazolo[3,4-b]pyridinyl, pyrazolo[3,4-c]pyridinyl, pyrazolo[4,3-c]pyridine, pyrazolo[4,3-b]pyridinyl, tetrazolyl, chromane, 2,3-dihydrobenzo[b][1,4]dioxine, benzo[d][1,3]dioxole, 2,3-dihydrobenzofuran, tetrahydroquinoline, 2,3-dihydrobenzo[b][1,4]oxathiine, and others. In some embodiments, the heteroaryl is selected from thienyl, pyridinyl, furyl, pyrazolyl, imidazolyl, pyranyl, pyrazinyl, and pyrimidinyl.

As used herein, “halo”, “halide” or “halogen” is a chloro, bromo, fluoro, or iodo atom radical. In some embodiments, a halo is a chloro, bromo or fluoro. For example, a halide can be fluoro.

As used herein, “haloalkyl” means a hydrocarbon substituent, which is a linear or branched, alkyl, alkenyl or alkynyl substituted with one or more chloro, bromo, fluoro, and/or iodo atom(s). In some embodiments, a haloalkyl is a fluoroalkyls, wherein one or more of the hydrogen atoms have been substituted by fluoro. In some embodiments, haloalkyls are of 1 to about 3 carbons in length (e.g., 1 to about 2 carbons in length or 1 carbon in length). The term “haloalkylene” means a diradical variant of haloalkyl, and such diradicals may act as spacers between radicals, other atoms, or between a ring and another functional group.

As used herein, “heterocyclyl” means a nonaromatic cyclic ring system comprising at least one heteroatom in the ring system backbone. Heterocyclyls may include multiple fused rings. Heterocyclyls may be substituted or unsubstituted with one or more substituents. In some embodiments, heterocycles have 5-7 members. In six membered monocyclic heterocycles, the heteroatom(s) are selected from one to three of O, N or S, and wherein when the heterocycle is five membered, it can have one or two heteroatoms selected from O, N, or S. Examples of heterocyclyl include azirinyl, aziridinyl, azetidinyl, oxetanyl, thietanyl, 1,4,2-dithiazolyl, dihydropyridinyl, 1,3-dioxanyl, 1,4-dioxanyl, 1,3-dioxolanyl, morpholinyl, thiomorpholinyl, piperazinyl, pyranyl, pyrrolidinyl, tetrahydrofuryl, tetrahydropyridinyl, oxazinyl, thiazinyl, thiinyl, thiazolidinyl, isothiazolidinyl, oxazolidinyl, isoxazolidinyl, piperidinyl, pyrazolidinyl imidazolidinyl, thiomorpholinyl, and others. In some embodiments, the heterocyclyl is selected from azetidinyl, morpholinyl, piperazinyl, pyrrolidinyl, and tetrahydropyridinyl.

As used herein, “monocyclic heterocyclyl” means a single nonaromatic cyclic ring comprising at least one heteroatom in the ring system backbone. Heterocyclyls may be substituted or unsubstituted with one or more substituents. In some embodiments, heterocycles have 5-7 members. In six membered monocyclic heterocycles, the heteroatom(s) are selected from one to three of O, N or S, and wherein when the heterocycle is five membered, it can have one or two heteroatoms selected from O, N, or S. Examples of heterocyclyl include azirinyl, aziridinyl, azetidinyl, oxetanyl, thietanyl, 1,4,2-dithiazolyl, dihydropyridinyl, 1,3-dioxanyl, 1,4-dioxanyl, 1,3-dioxolanyl, morpholinyl, thiomorpholinyl, piperazinyl, pyranyl, pyrrolidinyl, tetrahydrofuryl, tetrahydropyridinyl, oxazinyl, thiazinyl, thiinyl, thiazolidinyl, isothiazolidinyl, oxazolidinyl, isoxazolidinyl, piperidinyl, pyrazolidinyl imidazolidinyl, thiomorpholinyl, and others.

As used herein, “spirocyclic heterocyclyl” means a nonaromatic bicyclic ring system comprising at least one heteroatom in the ring system backbone and with the rings connected through just one atom. Spirocyclic heterocyclyls may be substituted or unsubstituted with one or more substituents. In some embodiments, spirocyclic heterocycles have 5-11 members with the heteroatom(s) being selected from one to five of O, N or S. Examples of spirocyclic heterocyclyl include 2-azaspiro[2.2]pentane, 4-azaspiro[2.5]octane, 1-azaspiro[3.5]nonane, 2-azaspiro[3.5]nonane, 2-azaspiro[4.4]nonane, 6-azaspiro[2.6]nonane, 1,7-diazaspiro[4.5]decane, 2,5-diazaspiro[3.6]decane, and the like.

The term “substituted” refers to moieties having substituents replacing a hydrogen on one or more non-hydrogen atoms of the molecule. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. Substituents can include, for example, —(C₁₋₉ alkyl) optionally substituted with one or more of hydroxyl, —NH₂, —NH(C₁₋₃ alkyl), and —N(C₁₋₃ alkyl)₂; —(C₁₋₉ haloalkyl); a halide; a hydroxyl; a carbonyl [such as —C(O)OR, and —C(O)R]; a thiocarbonyl [such as —C(S)OR, —C(O)SR, and —C(S)R]; —(C₁₋₉ alkoxyl) optionally substituted with one or more of halide, hydroxyl, —NH₂, —NH(C₁₋₃ alkyl), and —N(C₁₋₃ alkyl)₂; —OPO(OH)₂; a phosphonate [such as —PO(OH)₂ and —PO(OR′)₂]; —OPO(OR′)R″; —NRR′; —C(O)NRR′; —C(NR)NR′R″; —C(NR′)R″; a cyano; a nitro; an azido; —SH; —S—R; —OSO₂(OR); a sulfonate [such as —SO₂(OH) and —SO₂(OR)]; —SO₂NR′R″; and —SO₂R; in which each occurrence of R, R′ and R″ are independently selected from H; —(C₁₋₉ alkyl); C₆₋₁₀ aryl optionally substituted with from 1-3R′″; 5-10 membered heteroaryl having from 1-4 heteroatoms independently selected from N, O, and S and optionally substituted with from 1-3 R′″; C₃₋₇ carbocyclyl optionally substituted with from 1-3 R′″; and 3-8 membered heterocyclyl having from 1-4 heteroatoms independently selected from N, O, and S and optionally substituted with from 1-3 R′″; wherein each R′″ is independently selected from —(C₁₋₆ alkyl), —(C₁₋₆ haloalkyl), a halide (e.g., F), a hydroxyl, —C(O)OR, —C(O)R, —(C₁₋₆ alkoxyl), —NRR′, —C(O)NRR′, and a cyano, in which each occurrence of R and R′ is independently selected from H and —(C₁₋₆ alkyl). In some embodiments, the substituent is selected from —(C₁₋₆ alkyl), —(C₁₋₆ haloalkyl), a halide (e.g., F), a hydroxyl, —C(O)OR, —C(O)R, —(C₁₋₆ alkoxyl), —NRR′, —C(O)NRR′, and a cyano, in which each occurrence of R and R′ is independently selected from H and —(C₁₋₆ alkyl).

As used herein, when two groups are indicated to be “linked” or “bonded” to form a “ring”, it is to be understood that a bond is formed between the two groups and may involve replacement of a hydrogen atom on one or both groups with the bond, thereby forming a carbocyclyl, heterocyclyl, aryl, or heteroaryl ring. The skilled artisan will recognize that such rings can and are readily formed by routine chemical reactions. In some embodiments, such rings have from 3-7 members, for example, 5 or 6 members.

The skilled artisan will recognize that some structures described herein may be resonance forms or tautomers of compounds that may be fairly represented by other chemical structures, even when kinetically, the artisan recognizes that such structures are only a very small portion of a sample of such compound(s). Such compounds are clearly contemplated within the scope of this disclosure, though such resonance forms or tautomers are not represented herein.

The compounds provided herein may encompass various stereochemical forms. The compounds also encompass diastereomers as well as optical isomers, e.g., mixtures of enantiomers including racemic mixtures, as well as individual enantiomers and diastereomers, which arise as a consequence of structural asymmetry in certain compounds. Separation of the individual isomers or selective synthesis of the individual isomers is accomplished by application of various methods which are well known to practitioners in the art. Unless otherwise indicated, when a disclosed compound is named or depicted by a structure without specifying the stereochemistry and has one or more chiral centers, it is understood to represent all possible stereoisomers of the compound.

The term “administration” or “administering” refers to a method of providing a dosage of a compound or pharmaceutical composition to a vertebrate or invertebrate, including a mammal, a bird, a fish, or an amphibian, where the method is, e.g., orally, subcutaneously, intravenously, intralymphatic, intranasally, topically, transdermally, intraperitoneally, intramuscularly, intrapulmonarilly, vaginally, rectally, ontologically, neuro-otologically, intraocularly, subconjuctivally, via anterior eye chamber injection, intravitreally, intraperitoneally, intrathecally, intracystically, intrapleurally, via wound irrigation, intrabuccally, intra-abdominally, intra-articularly, intra-aurally, intrabronchially, intracapsularly, intrameningeally, via inhalation, via endotracheal or endobronchial instillation, via direct instillation into pulmonary cavities, intraspinally, intrasynovially, intrathoracically, via thoracostomy irrigation, epidurally, intratympanically, intracisternally, intravascularly, intraventricularly, intraosseously, via irrigation of infected bone, or via application as part of any admixture with a prosthetic device. The method of administration can vary depending on various factors, e.g., the components of the pharmaceutical composition, the site of the disease, the disease involved, and the severity of the disease.

A “diagnostic” as used herein is a compound, method, system, or device that assists in the identification or characterization of a health or disease state. The diagnostic can be used in standard assays as is known in the art.

The term “mammal” is used in its usual biological sense. Thus, it specifically includes humans, cattle, horses, monkeys, dogs, cats, mice, rats, cows, sheep, pigs, goats, and non-human primates, but also includes many other species.

The term “pharmaceutically acceptable carrier”, “pharmaceutically acceptable diluent” or “pharmaceutically acceptable excipient” includes any and all solvents, co-solvents, complexing agents, dispersion media, coatings, isotonic and absorption delaying agents and the like which are not biologically or otherwise undesirable. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions. In addition, various adjuvants such as are commonly used in the art may be included. These and other such compounds are described in the literature, e.g., in the Merck Index, Merck & Company, Rahway, N.J. Considerations for the inclusion of various components in pharmaceutical compositions are described, e.g., in Gilman et al. (Eds.) (2010); Goodman and Gilman's: The Pharmacological Basis of Therapeutics, 12th Ed., The McGraw-Hill Companies.

The term “pharmaceutically acceptable salt” refers to salts that retain the biological effectiveness and properties of the compounds provided herein and, which are not biologically or otherwise undesirable. In many cases, the compounds provided herein are capable of forming acid and/or base salts by virtue of the presence of amino and/or carboxyl groups or groups similar thereto. Many such salts are known in the art, for example, as described in WO 87/05297. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium, and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine.

“Solvate” refers to the compound formed by the interaction of a solvent and a compound as provided herein or a salt thereof. Suitable solvates are pharmaceutically acceptable solvates including hydrates.

“Patient” as used herein, means a human or a non-human mammal, e.g., a dog, a cat, a mouse, a rat, a cow, a sheep, a pig, a goat, a non-human primate, or a bird, e.g., a chicken, as well as any other vertebrate or invertebrate. In some embodiments, the patient is a human.

A “therapeutically effective amount” of a compound as provided herein is one which is sufficient to achieve the desired physiological effect and may vary according to the nature and severity of the disease condition, and the potency of the compound. “Therapeutically effective amount” is also intended to include one or more of the compounds of Formula I in combination with one or more other agents that are effective to treat the diseases and/or conditions described herein. The combination of compounds can be a synergistic combination. Synergy, as described, for example, by Chou and Talalay, Advances in Enzyme Regulation (1984), 22, 27-55, occurs when the effect of the compounds when administered in combination is greater than the additive effect of the compounds when administered alone as a single agent. In general, a synergistic effect is most clearly demonstrated at sub-optimal concentrations of the compounds. It will be appreciated that different concentrations may be employed for prophylaxis than for treatment of an active disease. This amount can further depend upon the patient's height, weight, sex, age and medical history.

A therapeutic effect relieves, to some extent, one or more of the symptoms of the disease.

“Treat,” “treatment,” or “treating,” as used herein refers to administering a compound or pharmaceutical composition as provided herein for therapeutic purposes. The term “therapeutic treatment” refers to administering treatment to a patient already suffering from a disease thus causing a therapeutically beneficial effect, such as ameliorating existing symptoms, ameliorating the underlying metabolic causes of symptoms, postponing or preventing the further development of a disorder, and/or reducing the severity of symptoms that will or are expected to develop.

The term “cure” refers to either the complete elimination of all forms of the virus (e.g., persistent infectious HIV) from all tissues in the body (“sterilizing cure” or “complete eradication”) or the immunological control of the virus (e.g., persistent HIV) (“functional cure”) even in the presence of viral (e.g., HIV) reservoirs in the body. For example, the goal of HIV eradication is to eliminate all forms of HIV proviruses that can be reactivated to replicate (“replication-competent” viruses) as measured with viral outgrowth assays. A functional cure is considered more achievable and does not require elimination or reduction of the viral reservoirs, but allows for discontinuation of therapy without viral rebounds as the immune system can control viral replication alone.

As used herein, the term “viral infection” describes a diseased state in which a virus invades healthy cells, uses the cell's reproductive machinery to multiply or replicate and ultimately lyse the cell resulting in cell death, release of viral particles and the infection of other cells by the newly produced progeny viruses. Latent infection by certain viruses is also a possible result of viral infection.

As used herein, the term “treating viral infections” means to inhibit the replication of the particular virus, to inhibit viral transmission, and to ameliorate or alleviate the symptoms of the disease caused by the viral infection. The treatment is considered “therapeutic” if there is a reduction in viral load, decrease in mortality and/or morbidity. “Preventing viral infections” means to prevent the virus from establishing itself in the host. A treatment is considered “prophylactic” if the subject is exposed to the virus, but does not become infected with the virus as a result of treatment.

“Latency” means a concept describing 1) where a virus genome (provirus) is integrated into the DNA of a host cell but remains transcriptionally inactive, 2) the dormant state of viral activity within a population of cells, wherein viral production, viral packaging, viral replication and virus-mediated host cell lysis or death does not occur, or occurs at a very low frequency, or 3) the down-regulation or absence of gene expression within an infected cell. “Latent” in the viral context can mean that the viral genome has integrated into the host cell genome without subsequent viral packaging of the viral genome into a viral capsid or other virus structure, which then causes the host cell to lyse, releasing infectious viral particles that are free to infect other cells in the host. Latency can be spontaneously reversed, induced under physiological conditions that occur in vivo, or pharmacologically by administration of latency reversing agents that lead to the onset of viral antigen expression and viral replication. “Latency” in the context of the viral life cycle can also refer to a virus' “lysogenic phase.” In contrast, a virus is in the “lytic” phase if the viral genomes are packaged into a capsid or other viral structure, ultimately leading to lysis of the host cell and release of newly packaged viruses into the host.

Compounds

The compounds and compositions described herein can be used as inhibitors of vial mediated downmodulation of major histocompatibility complex I (MHC-I) for the treatment of patients infected with primate lentiviruses (e.g., human immunodeficiency viruses (HIV-1 and HIV-2) and simian immunodeficiency virus (SIV)).

Some embodiments of the present disclosure include compounds of Formula I.

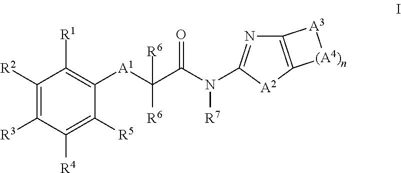

or enantiomers, diastereomers, tautomers, prodrugs, and pharmaceutically acceptable salts thereof.

In some embodiments of Formula I, R¹ and R² are taken together to form a ring which is selected from the group consisting of heteroaryl optionally substituted with 1-4 (e.g., 1-3. 1-2. 1) R⁸ and heterocyclyl optionally substituted with 1-4 (e.g., 1-3. 1-2. 1) R⁹.

In some embodiments of Formula I,

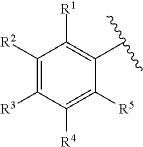

is selected from the group consisting of

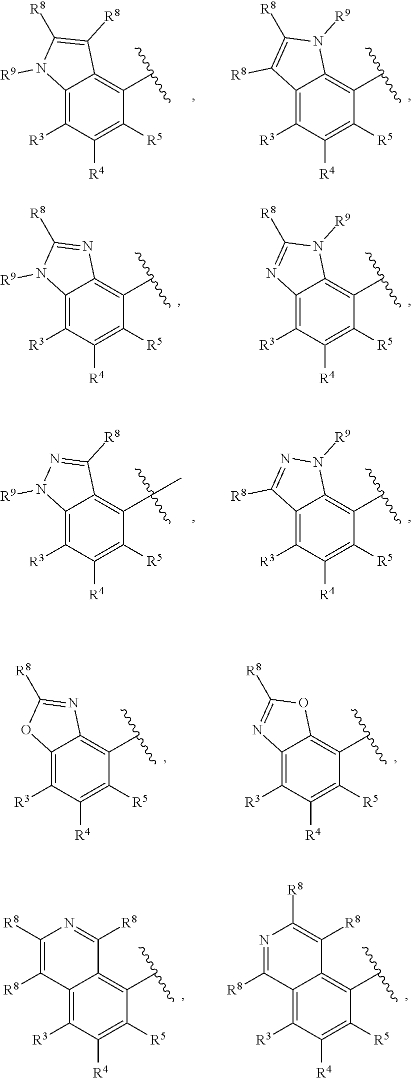

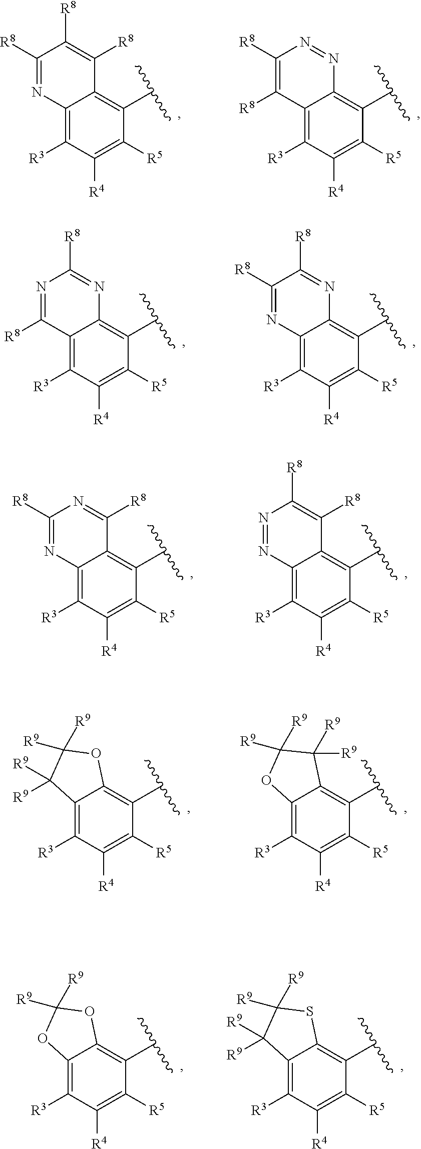

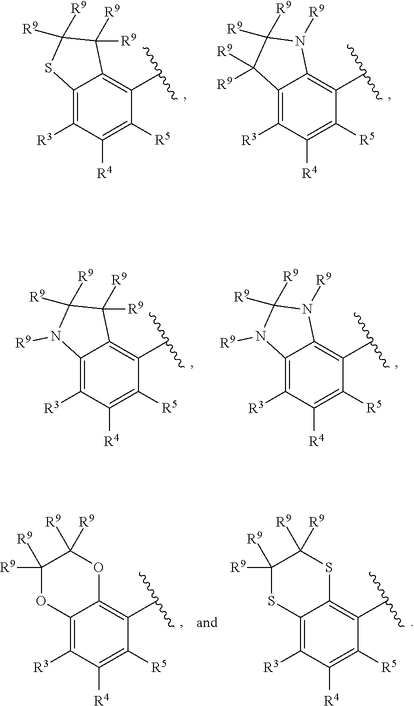

In some embodiments of Formula I,

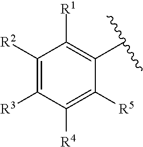

is selected from the group consisting of

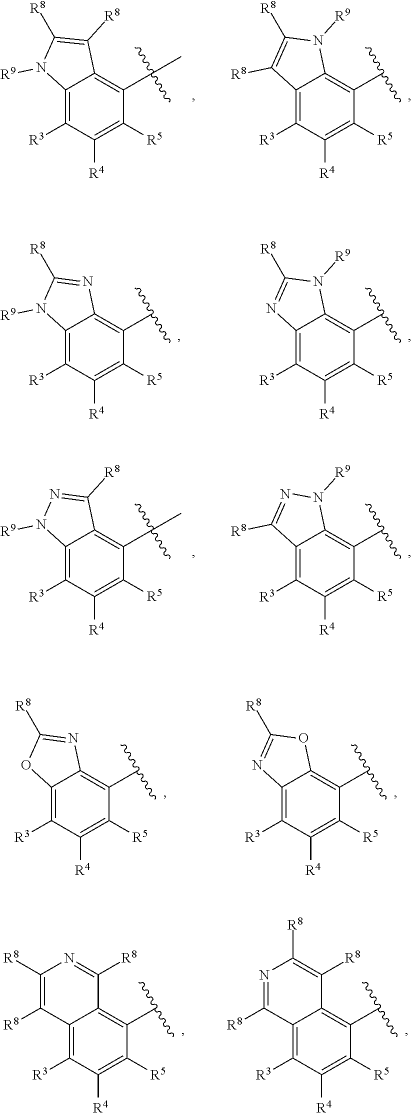

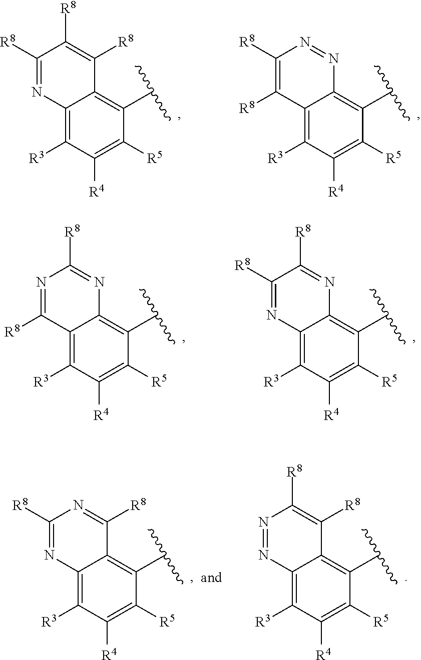

In some embodiments of Formula I,

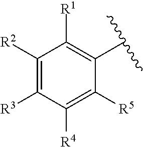

is selected from the group consisting of

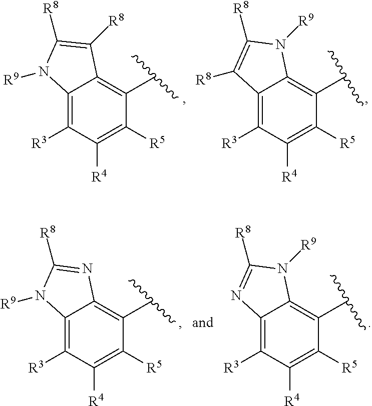

In some embodiments of Formula I,

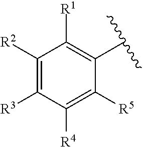

is selected from the group consisting of

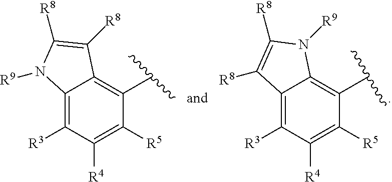

In some embodiments of Formula I,

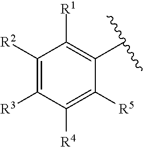

is selected from the group consisting of

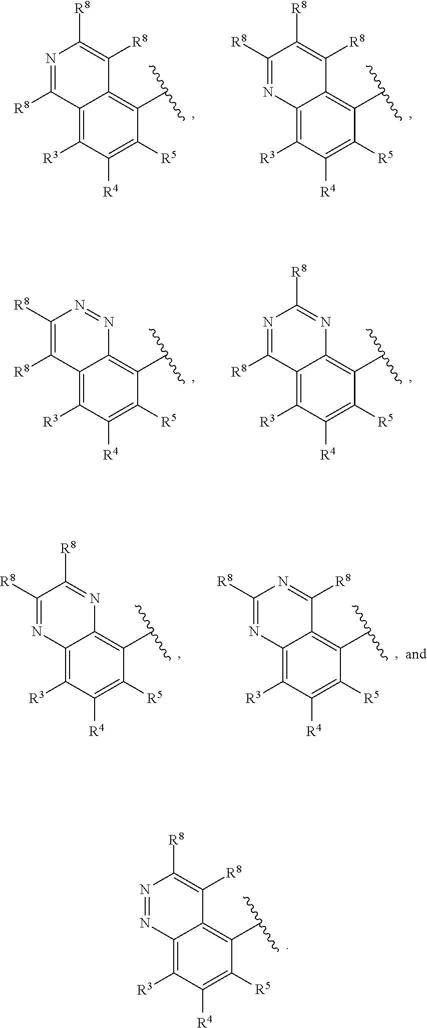

In some embodiments of Formula I,

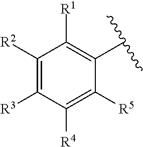

is selected from the group consisting of

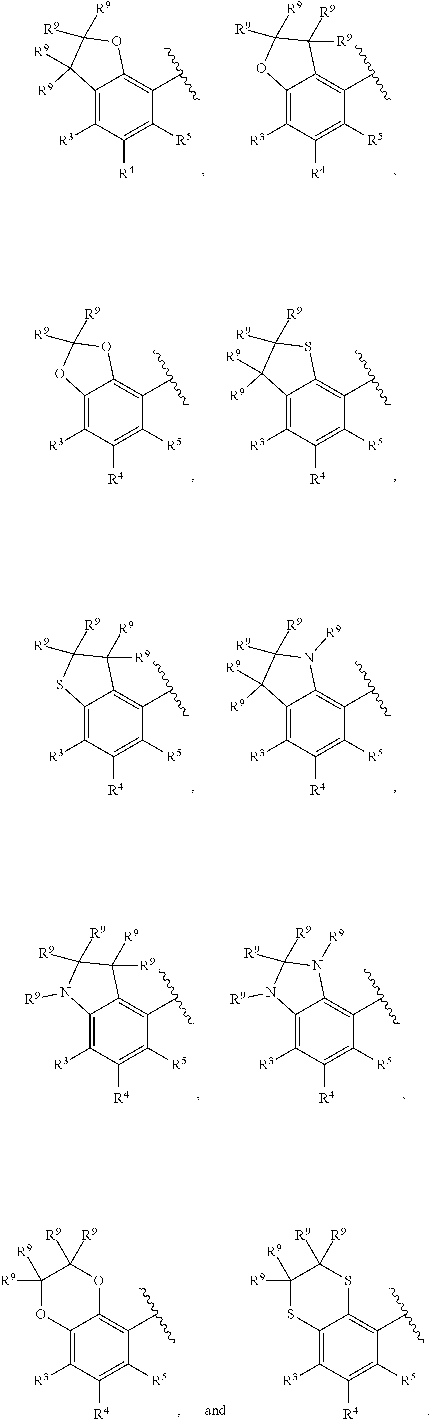

In some embodiments of Formula I,

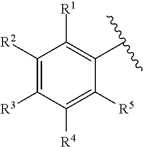

is selected from the group consisting of

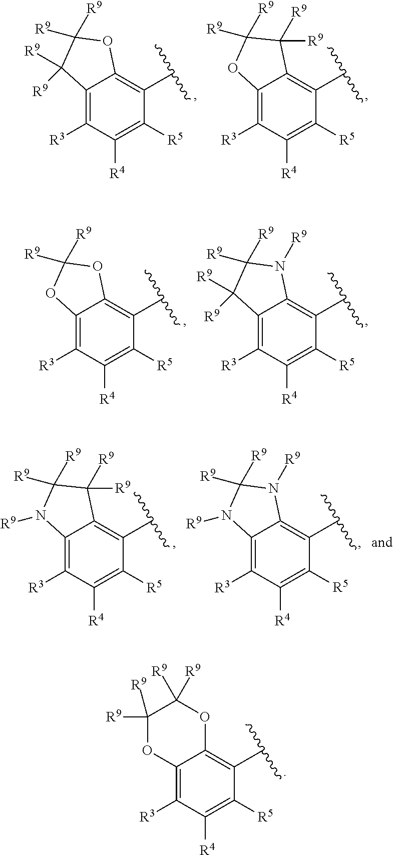

In some embodiments of Formula I,

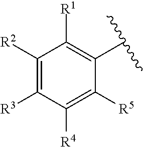

is selected from the group consisting of

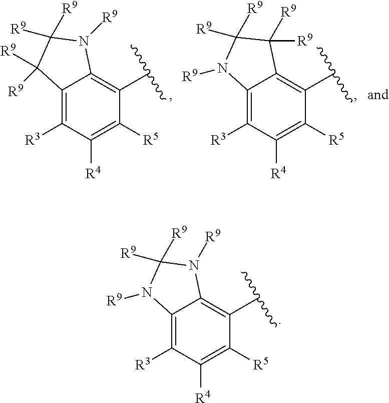

In some embodiments of Formula I,

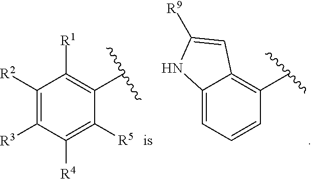

In some embodiments of Formula I,

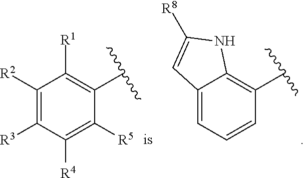

In some embodiments of Formula I,

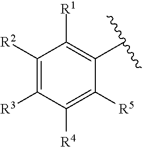

is selected from the group consisting of

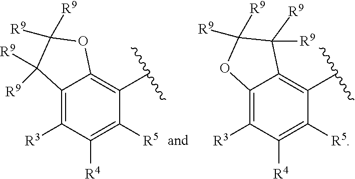

In some embodiments of Formula I,

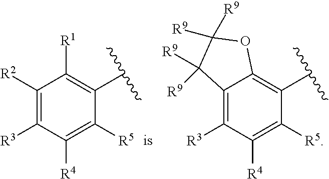

In some embodiments of Formula I,

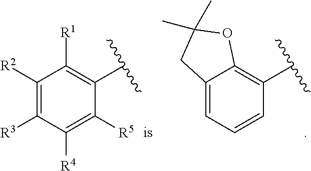

In some embodiments of Formula I,

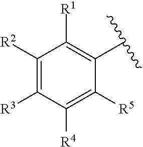

is selected from the group consisting of

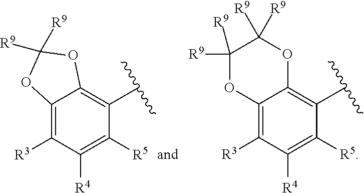

In some embodiments of Formula I, there is the proviso that

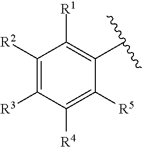

does not form

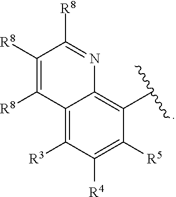

In some embodiments of Formula I, each R⁸ is independently selected from the group consisting of halide (e.g., F, Cl, Br, I), unsubstituted —(C₁₋₉ alkyl) (e.g., C₁₋₈, C₁₋₇, C₁₋₆, C₁₋₅, C₁₋₄, C₁₋₃, C₁₋₂, C₁), unsubstituted —(C₂₋₉ alkenyl) (e.g., C₂₋₈, C₂₋₇, C₂₋₆, C₂₋₅, C₂₋₄, C₂₋₃, C₂), unsubstituted —(C₂₋₉ alkynyl) (e.g., C₂₋₈, C₂₋₇, C₂₋₆, C₂₋₅, C₂₋₄, C₂₋₃, C₂), unsubstituted —(C₁₋₉ haloalkyl) (e.g., C₁₋₈, C₁₋₇, C₁₋₆, C₁₋₅, C₁₋₄, C₁₋₃, C₁₋₂, C₁), —OR¹⁰, and —CN;

In some embodiments of Formula I, each R⁸ is independently selected from the group consisting of F, Cl, Br, unsubstituted —(C₁₋₄ alkyl) (e.g., C₁₋₃, C₁₋₂, C₁), unsubstituted —(C₂₋₄ alkenyl) (e.g., C₂₋₃, C₂), unsubstituted —(C₂₋₄ alkynyl) (e.g., C₂₋₃, C₂), unsubstituted —(C₁₋₄ haloalkyl) (e.g., C₁₋₃, C₁₋₂, C₁), —O(C₁₋₄ alkyl) (e.g., C₁₋₃, C₁₋₂, C₁), and —CN;

In some embodiments of Formula I, each R⁸ is independently selected from the group consisting of F, Cl, unsubstituted —(C₁₋₃ alkyl) (e.g., C₁₋₂, C₁), unsubstituted —(C₂₋₃ alkenyl) (e.g., C₂), unsubstituted —(C₂₋₃ alkynyl) (e.g., C₂), unsubstituted —(C₁₋₃ haloalkyl) (e.g., C₁₋₂, C₁), —O(C₁₋₃ alkyl) (e.g., C₁₋₂, C₁), and —CN;

In some embodiments of Formula I, each R⁸ is independently selected from the group consisting of F, unsubstituted —(C₁₋₂ alkyl) (e.g., Me), unsubstituted —(C₂ alkenyl), unsubstituted —(C₂ alkynyl), unsubstituted —(C₁₋₂ haloalkyl) (e.g., C₁), —O(C₁₋₂ alkyl) (e.g., Me), and —CN;

In some embodiments of Formula I, each R⁸ is independently selected from the group consisting of F, Me, —CF₃, —OMe, and —CN;

In some embodiments of Formula I, each R⁹ is independently selected from the group consisting of halide (e.g., F, Cl, Br, I), unsubstituted —(C₁₋₉ alkyl) (e.g., C₁₋₈, C₁₋₇, C₁₋₆, C₁₋₅, C₁₋₄, C₁₋₃, C₁₋₂, C₁), unsubstituted —(C₂₋₉ alkenyl) (e.g., C₂₋₈, C₂₋₇, C₂₋₆, C₂₋₅, C₂₋₄, C₂₋₃, C₂), unsubstituted —(C₂₋₉ alkynyl) (e.g., C₂₋₈, C₂₋₇, C₂₋₆, C₂₋₅, C₂₋₄, C₂₋₃, C₂), unsubstituted —(C₁₋₉ haloalkyl) (e.g., C₁₋₈, C₁₋₇, C₁₋₆, C₁₋₅, C₁₋₄, C₁₋₃, C₁₋₂, C₁), and —CN;

In some embodiments of Formula I, each R⁹ is independently selected from the group consisting of F, Cl, unsubstituted —(C₁₋₃ alkyl) (e.g., C₁₋₂, C₁), unsubstituted —(C₂₋₃ alkenyl) (e.g., C₂), unsubstituted —(C₂₋₃ alkynyl) (e.g., C₂), unsubstituted —(C₁₋₃ haloalkyl) (e.g., C₁₋₂, C₁), —O(C₁₋₃ alkyl) (e.g., C₁₋₂, C₁), and —CN;

In some embodiments of Formula I, each R⁹ is independently selected from the group consisting of F, unsubstituted —(C₁₋₂ alkyl) (e.g., Me), unsubstituted —(C₂ alkenyl), unsubstituted —(C₂ alkynyl), unsubstituted —(C₁₋₂ haloalkyl) (e.g., C₁), —O(C₁₋₂ alkyl) (e.g., Me), and —CN;

In some embodiments of Formula I, each R⁹ is independently selected from the group consisting of F, Me, —CF₃, —OMe, and —CN.

For example, provided herein is a compound, or a pharmaceutically acceptable salt thereof, of Formula I:

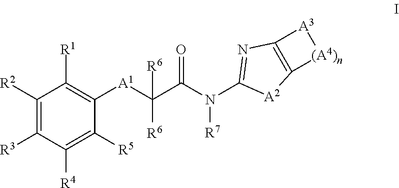

wherein:
R¹ and R² are taken together to form a ring which is selected from the group consisting of heteroaryl optionally substituted with 1-4 R⁸ and heterocyclyl optionally substituted with 1-4 R⁹;
R³, R⁴, and R⁵ are independently selected from the group consisting of H, halide, unsubstituted —(C₁₋₆ alkyl), unsubstituted —(C₁₋₄ haloalkyl), —OR¹⁰, and —CN;
each R⁶ is independently selected from the group consisting of H, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein;
R⁷ is selected from the group consisting of H, unsubstituted —(C₁₋₉ alkyl), unsubstituted —(C₃₋₉ alkenyl), unsubstituted —(C₃₋₉ alkynyl), unsubstituted —(C₁₋₉ haloalkyl), —(C₁₋₄ alkylene)C(═O)OR¹¹, —(C₁₋₄ alkylene)C(═O)N(R¹²)₂, —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein;
each R⁸ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR¹⁰, and —CN;
each R⁹ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
each R¹⁰ is independently selected from the group consisting of H, unsubstituted —(C₁₋₉ alkyl), unsubstituted —(C₃₋₉ alkenyl), unsubstituted —(C₃₋₉ alkynyl), and unsubstituted —(C₁₋₉ haloalkyl);
each R¹¹ is independently selected from the group consisting of H and unsubstituted —(C₁₋₉ alkyl);
each R¹² is independently selected from the group consisting of H and substituted or unsubstituted —(C₁₋₉ alkyl);
each R¹³ is independently selected from the group consisting of halide, unsubstituted —(C₁₋₉ alkyl), unsubstituted —(C₁₋₉ haloalkyl), and —OR¹⁰;
each R¹⁴ is independently selected from the group consisting of halide, unsubstituted —(C₁₋₉ alkyl), unsubstituted —(C₁₋₉ haloalkyl), and —OR¹⁰;
each R¹⁵ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁶ is independently selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
A¹ is selected from the group consisting of —C(R¹⁵)₂—, O, and S;
A² is selected from the group consisting of O and S;
A³ is selected from the group consisting of —C(R¹⁶)₂—, O and S;
each A⁴ is independently selected from the group consisting of —C(R¹⁶)₂—, O and S;
with the proviso that no more than two A³ and A⁴ are O or S and that two O or S are never adjacent;
n is 1, 2, 3, or 4; and
each p independently is 0 or 1.

In some embodiments, provided herein is a compound, or a pharmaceutically acceptable salt thereof, of Formula I:

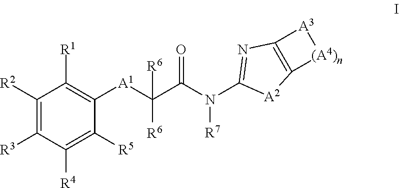

wherein:
R¹ and R² are taken together to form a ring which is selected from the group consisting of heteroaryl optionally substituted with 1-4 R⁸ and heterocyclyl optionally substituted with 1-4 R⁹;
with the proviso that

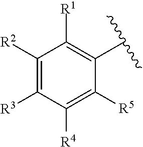

does not form

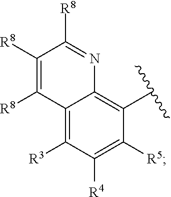

R³, R⁴, and R⁵ are independently selected from the group consisting of H, halide, unsubstituted —(C₁₋₆ alkyl), unsubstituted —(C₁₋₄ haloalkyl), —OR¹⁰, and —CN;
each R⁶ is independently selected from the group consisting of H, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein;
R⁷ is selected from the group consisting of H, unsubstituted —(C₁₋₉ alkyl), unsubstituted —(C₃₋₉ alkenyl), unsubstituted —(C₃₋₉ alkynyl), unsubstituted —(C₁₋₉ haloalkyl), —(C₁₋₄ alkylene)C(═O)OR¹¹, —(C₁₋₄ alkylene)C(═O)N(R¹²)₂, —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein;
each R⁸ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR¹⁰, and —CN;
each R⁹ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
each R¹⁰ is independently selected from the group consisting of H, unsubstituted —(C₁₋₉ alkyl), unsubstituted —(C₃₋₉ alkenyl), unsubstituted —(C₃₋₉ alkynyl), and unsubstituted —(C₁₋₉ haloalkyl);
each R¹¹ is independently selected from the group consisting of H and unsubstituted —(C₁₋₉ alkyl);
each R¹² is independently selected from the group consisting of H and substituted or unsubstituted —(C₁₋₉ alkyl);
each R¹³ is independently selected from the group consisting of halide, unsubstituted —(C₁₋₉ alkyl), unsubstituted —(C₁₋₉ haloalkyl), and —OR¹⁰;
each R¹⁴ is independently selected from the group consisting of halide, unsubstituted —(C₁₋₉ alkyl), unsubstituted —(C₁₋₉ haloalkyl), and —OR¹⁰;
each R¹⁵ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁶ is independently selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
A¹ is selected from the group consisting of —C(R¹⁵)₂—, O, and S;
A² is selected from the group consisting of O and S;
A³ is selected from the group consisting of —C(R¹⁶)₂—, O and S;
each A⁴ is independently selected from the group consisting of —C(R¹⁶)₂—, O and S;
with the proviso that no more than two A³ and A⁴ are O or S and that two O or S are never adjacent;
n is 1, 2, 3, or 4;
each p independently is 0 or 1; and
with the proviso that Formula I is not a structure selected from the group consisting of:

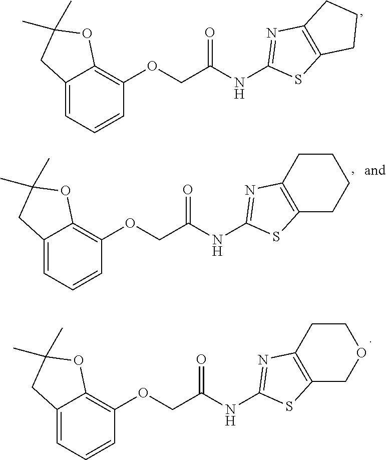

Some embodiments of the present disclosure include compounds of Formula II:

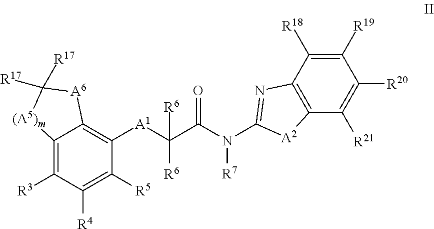

or enantiomers, diastereomers, tautomers, prodrugs, and pharmaceutically acceptable salts thereof.

In some embodiments of Formula II,

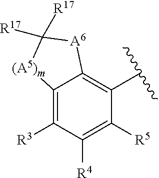

is selected from the group consisting of

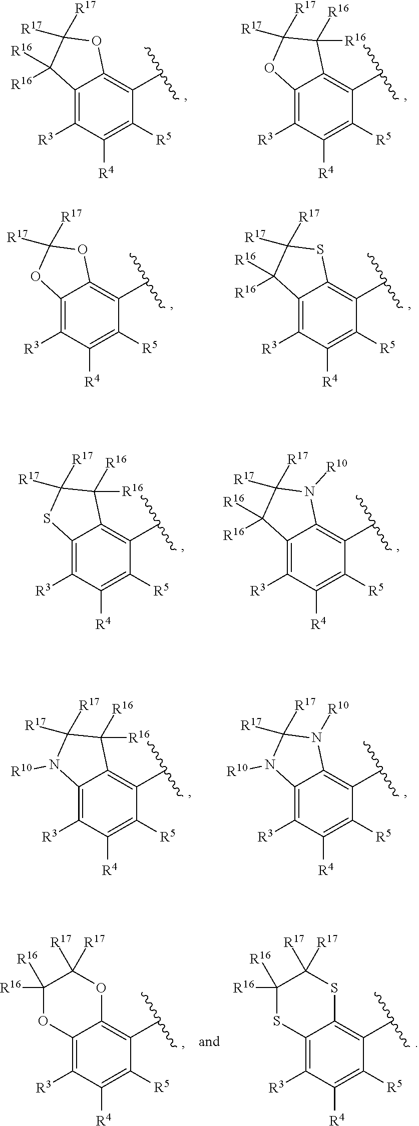

In some embodiments of Formula II,

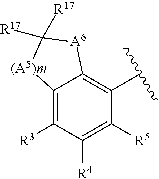

is selected from the group consisting of

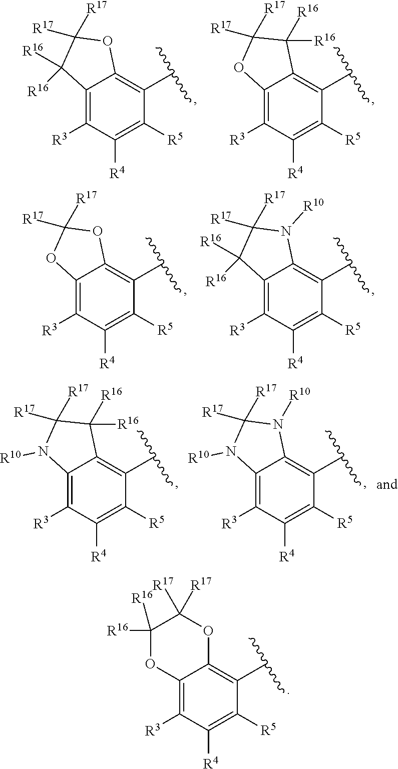

In some embodiments of Formula II,

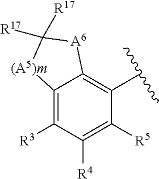

is selected from the group consisting of

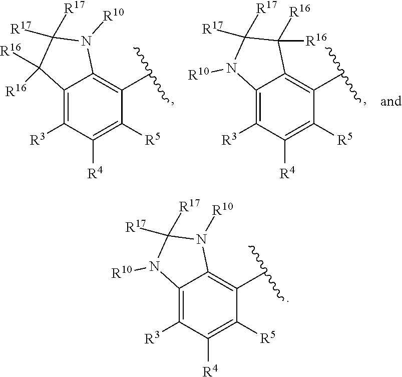

In some embodiments of Formula II,

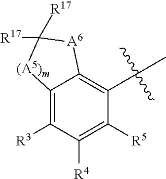

is selected from the group consisting of

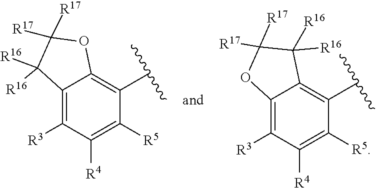

In some embodiments of Formula II,

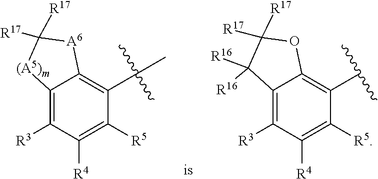

In some embodiments of Formula II,

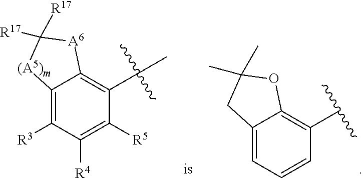

In some embodiments of Formula II,

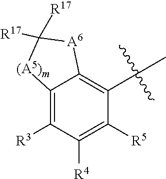

is selected from the group consisting of

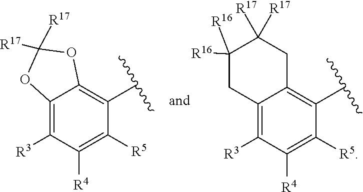

In some embodiments of Formula II, R¹⁸ is selected from the group consisting of H, halide (e.g., F, Cl, Br, I), unsubstituted —(C₁₋₉ alkyl) (e.g., C₁₋₈, C₁₋₇, C₁₋₆, C₁₋₅, C₁₋₄, C₁₋₃, C₁₋₂, C₁), unsubstituted —(C₂₋₉ alkenyl) (e.g., C₂₋₈, C₂₋₇, C₂₋₆, C₂₋₅, C₂₋₄, C₂₋₃, C₂), unsubstituted —(C₂₋₉ alkynyl) (e.g., C₂₋₈, C₂₋₇, C₂₋₆, C₂₋₅, C₂₋₄, C₂₋₃, C₂), unsubstituted —(C₁₋₉ haloalkyl) (e.g., C₁₋₈, C₁₋₇, C₁₋₆, C₁₋₅, C₁₋₄, C₁₋₃, C₁₋₂, C₁), —OR¹⁰, —(C₁₋₄ alkylene)ₚheteroaryl optionally substituted with 1-4 (e.g., 1-3. 1-2. 1) R²², —(C₁₋₄ alkylene)ₚaryl optionally substituted with 1-5 (e.g., 1-4, 1-3. 1-2. 1) R²³, and —CN; wherein each —(C₁₋₄ alkylene) is, independently, optionally substituted with one or more substituents as defined herein.

In some embodiments of Formula II, R¹⁸ is selected from the group consisting of unsubstituted —(C₁₋₃ haloalkyl) (e.g., C₁₋₂, C₁), —O(C₁₋₃ alkyl) (e.g., C₁₋₂, C₁), -heteroaryl optionally substituted with 1-4 (e.g., 1-3. 1-2. 1) R²², -aryl optionally substituted with 1-5 (e.g., 1-4, 1-3. 1-2. 1) R²³, and —CN;

In some embodiments of Formula II, R¹⁸ is selected from the group consisting of H, F, Cl, Br, unsubstituted —(C₁₋₄ alkyl) (e.g., C₁₋₃, C₁₋₂, C₁), unsubstituted —(C₂₋₄ alkenyl) (e.g., C₂₋₃, C₂), unsubstituted —(C₂₋₄ alkynyl) (e.g., C₂₋₃, C₂), unsubstituted —(C₁₋₄ haloalkyl) (e.g., C₁₋₃, C₁₋₂, C₁), —O(C₁₋₄ alkyl) (e.g., C₁₋₃, C₁₋₂, C₁), —(C₁₋₂ alkylene)ₚheteroaryl optionally substituted with 1-2 R²², —(C₁₋₂ alkylene)ₚphenyl optionally substituted with 1-2 R²³, and —CN; wherein each —(C₁₋₄ alkylene) is, independently, optionally substituted with one or more substituents as defined herein.

In some embodiments of Formula II, R¹⁸ is selected from the group consisting of H, F, Cl, unsubstituted —(C₁₋₂ alkyl) (e.g., Me), unsubstituted —(C₂ alkenyl), unsubstituted —(C₂ alkynyl), unsubstituted —(C₁₋₂ haloalkyl) (e.g., C₁), —O(C₁₋₂ alkyl) (e.g., Me), —(CH₂)ₚheteroaryl optionally substituted with 1-2 R²², —(CH₂)ₚphenyl optionally substituted with 1-2 R²³, and —CN.

In some embodiments of Formula II, R¹⁸ is selected from the group consisting of —CF₃, —OMe, -heteroaryl optionally substituted with 1 R²², -phenyl optionally substituted with 1-2 R²³, and —CN.

In some embodiments of Formula II, R¹⁸ is selected from the group consisting of H, F, Cl, Me, —CF₃, —OMe, and —CN.

In some embodiments of Formula II, R¹⁹ is selected from the group consisting of H, halide (e.g., F, Cl, Br, I), unsubstituted —(C₁₋₉ alkyl) (e.g., C₁₋₈, C₁₋₇, C₁₋₆, C₁₋₅, C₁₋₄, C₁₋₃, C₁₋₂, C₁), and unsubstituted —(C₁₋₆ haloalkyl) (e.g., C₁₋₈, C₁₋₇, C₁₋₆, C₁₋₅, C₁₋₄, C₁₋₃, C₁₋₂, C₁).

In some embodiments of Formula II, R¹⁹ is selected from the group consisting of H, F, Cl, Br, unsubstituted —(C₁₋₄ alkyl), and unsubstituted —(C₁₋₄ haloalkyl).

In some embodiments of Formula II, R¹⁹ is selected from the group consisting of H, F, Cl, unsubstituted —(C₁₋₂ alkyl), and unsubstituted —(C₁₋₂ haloalkyl).

In some embodiments of Formula II, R¹⁹ is selected from the group consisting of H, F, Cl, Me, and —CF₃.

In some embodiments of Formula II, R²⁰ is selected from the group consisting of H, halide (e.g., F, Cl, Br, I), unsubstituted —(C₁₋₉ alkyl) (e.g., C₁₋₈, C₁₋₇, C₁₋₆, C₁₋₅, C₁₋₄, C₁₋₃, C₁₋₂, C₁), and unsubstituted —(C₁₋₆ haloalkyl) (e.g., C₁₋₈, C₁₋₇, C₁₋₆, C₁₋₅, C₁₋₄, C₁₋₃, C₁₋₂, C₁).

In some embodiments of Formula II, R²⁰ is selected from the group consisting of H, F, Cl, Br, unsubstituted —(C₁₋₄ alkyl) (e.g., C₁₋₃, C₁₋₂, C₁), and unsubstituted —(C₁₋₄ haloalkyl) (e.g., C₁₋₃, C₁₋₂, C₁).

In some embodiments of Formula II, R²⁰ is selected from the group consisting of H, F, Cl, unsubstituted —(C₁₋₂ alkyl), and unsubstituted —(C₁₋₂ haloalkyl).

In some embodiments of Formula II, R²⁰ is selected from the group consisting of H, F, Cl, Me, and —CF₃.

In some embodiments of Formula II, R²¹ is selected from the group consisting of H, halide (e.g., F, Cl, Br, I), unsubstituted —(C₁₋₉ alkyl) (e.g., C₁₋₈, C₁₋₇, C₁₋₆, C₁₋₅, C₁₋₄, C₁₋₃, C₁₋₂, C₁), and unsubstituted —(C₁₋₆ haloalkyl) (e.g., C₁₋₈, C₁₋₇, C₁₋₆, C₁₋₅, C₁₋₄, C₁₋₃, C₁₋₂, C₁).

In some embodiments of Formula II, R²¹ is selected from the group consisting of H, F, Cl, Br, unsubstituted —(C₁₋₄ alkyl) (e.g., C₁₋₃, C₁₋₂, C₁), and unsubstituted —(C₁-4 haloalkyl) (e.g., C₁₋₃, C₁₋₂, C₁).

In some embodiments of Formula II, R²¹ is selected from the group consisting of H, F, Cl, unsubstituted —(C₁₋₂ alkyl) (e.g., Me), and unsubstituted —(C₁₋₂ haloalkyl) (e.g., C₁).

In some embodiments of Formula II, R²¹ is selected from the group consisting of H, F, Cl, Me, and —CF₃.

In some embodiments of Formula II, R¹⁹, R²⁰, and R²¹ are independently selected from the group consisting of H and halide (e.g., F, Cl, Br, I).

In some embodiments of Formula II, each R²² is independently selected from the group consisting of halide (e.g., F, Cl, Br, I), unsubstituted —(C₁₋₉ alkyl) (e.g., C₁₋₈, C₁₋₇, C₁₋₆, C₁₋₅, C₁₋₄, C₁₋₃, C₁₋₂, C₁), and unsubstituted —(C₁₋₆ haloalkyl) (e.g., C₁₋₈, C₁₋₇, C₁₋₆, C₁₋₅, C₁₋₄, C₁₋₃, C₁₋₂, C₁), and —CN.

In some embodiments of Formula II, R²² is selected from the group consisting of F, Cl, Br, unsubstituted —(C₁₋₄ alkyl) (e.g., C₁₋₃, C₁₋₂, C₁), unsubstituted —(C₁₋₄ haloalkyl) (e.g., C₁₋₃, C₁₋₂, C₁), and —CN.

In some embodiments of Formula II, R²² is selected from the group consisting of F, Cl, unsubstituted —(C₁₋₂ alkyl) (e.g., Me), and unsubstituted —(C₁₋₂ haloalkyl) (e.g., C₁), and —CN.

In some embodiments of Formula II, R²² is selected from the group consisting of F, Cl, Me, —CF₃, and —CN.

In some embodiments of Formula II, R²² is selected from the group consisting of methyl and halide (e.g., F, Cl, Br, I).

In some embodiments of Formula II, each R²³ is independently selected from the group consisting of halide (e.g., F, Cl, Br, I), unsubstituted —(C₁₋₉ alkyl) (e.g., C₁₋₈, C₁₋₇, C₁₋₆, C₁₋₅, C₁₋₄, C₁₋₃, C₁₋₂, C₁), and unsubstituted —(C₁₋₆ haloalkyl) (e.g., C₁₋₈, C₁₋₇, C₁₋₆, C₁₋₅, C₁₋₄, C₁₋₃, C₁₋₂, C₁), and —CN.

In some embodiments of Formula II, R²³ is selected from the group consisting of F, Cl, Br, unsubstituted —(C₁₋₄ alkyl) (e.g., C₁₋₃, C₁₋₂, C₁), unsubstituted —(C₁₋₄ haloalkyl) (e.g., C₁₋₃, C₁₋₂, C₁), and —CN.

In some embodiments of Formula II, R²³ is selected from the group consisting of F, Cl, unsubstituted —(C₁₋₂ alkyl) (e.g., Me), and unsubstituted —(C₁₋₂ haloalkyl) (e.g., C₁), and —CN.

In some embodiments of Formula II, R²³ is selected from the group consisting of F, Cl, Me, —CF₃, and —CN.

In some embodiments of Formula II, R²³ is selected from the group consisting of methyl, halide (e.g., F, Cl, Br, I), and —CN.

For example, provided herein is a compound, or a pharmaceutically acceptable salt thereof, of Formula II:

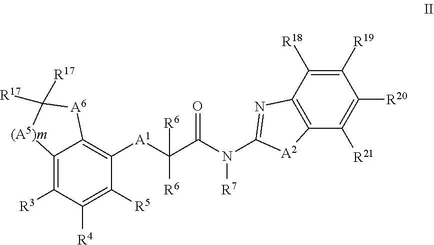

wherein:
R³, R⁴, and R⁵ are independently selected from the group consisting of H, halide, unsubstituted —(C₁₋₆ alkyl), unsubstituted —(C₁₋₄ haloalkyl), —OR¹⁰, and —CN;
each R⁶ is independently selected from the group consisting of H, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
R⁷ is selected from the group consisting of H, unsubstituted —(C₁₋₉ alkyl), unsubstituted —(C₃₋₉ alkenyl), unsubstituted —(C₃₋₉ alkynyl), unsubstituted —(C₁₋₉ haloalkyl), —(C₁₋₄ alkylene)C(═O)OR¹¹, —(C₁₋₄ alkylene)C(═O)N(R¹²)₂, —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein;
each R¹⁰ is independently selected from the group consisting of H, unsubstituted —(C₁₋₉ alkyl), unsubstituted —(C₃₋₉ alkenyl), unsubstituted —(C₃₋₉ alkynyl), and unsubstituted —(C₁₋₉ haloalkyl);
R¹¹ is selected from the group consisting of H and unsubstituted —(C₁₋₉ alkyl);
each R¹² is independently selected from the group consisting of H and substituted or unsubstituted —(C₁₋₉ alkyl);
each R¹³ is independently selected from the group consisting of halide, unsubstituted —(C₁₋₉ alkyl), unsubstituted —(C₁₋₉ haloalkyl), and —OR¹⁰;
each R¹⁴ is independently selected from the group consisting of halide, unsubstituted —(C₁₋₉ alkyl), unsubstituted —(C₁₋₉ haloalkyl), and —OR¹⁰;
each R¹⁵ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁶ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁷ is independently selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R¹⁸ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR¹⁰, —(C₁₋₄ alkylene)ₚheteroaryl optionally substituted with 1-4 R²², —(C₁₋₄ alkylene)ₚaryl optionally substituted with 1-5 R²³, and —CN; wherein each —(C₁₋₄ alkylene) is, independently, optionally substituted with one or more substituents as defined herein;
R¹⁹ is selected from the group consisting of H, halide, unsubstituted —(C₁₋₉ alkyl), and unsubstituted —(C₁₋₆ haloalkyl);
R²⁰ is selected from the group consisting of H, halide, unsubstituted —(C₁₋₉ alkyl), and unsubstituted —(C₁₋₆ haloalkyl);
R²¹ is selected from the group consisting of H, halide, unsubstituted —(C₁₋₉ alkyl), and unsubstituted —(C₁₋₆ haloalkyl);
each R²² is independently selected from the group consisting of halide, unsubstituted —(C₁₋₉ alkyl), unsubstituted —(C₁₋₉ haloalkyl), and —CN;
each R²³ is independently selected from the group consisting of halide, unsubstituted —(C₁₋₉ alkyl), unsubstituted —(C₁₋₉ haloalkyl), and —CN;
A¹ is selected from the group consisting of —C(R¹⁵)₂—, O, and S;
A² is selected from the group consisting of O and S;
each A⁵ is independently selected from the group consisting of —C(R¹⁶)₂—, —N(R¹⁰)—, O, and S;
with the proviso that two A⁵ are not both N, O or S;
A⁶ is selected from the group consisting of —C(R¹⁶)₂—, —N(R¹⁰)—, O, and S;
with the proviso that A⁵ and A⁶ cannot both be C(R¹⁶)₂;
m is 1 or 2; and
each p independently is 0 or 1.

In some embodiments, provided herein is a compound, or a pharmaceutically acceptable salt thereof, of Formula II:

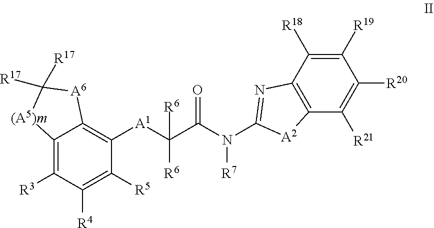

wherein:
R³, R⁴, and R⁵ are independently selected from the group consisting of H, halide, unsubstituted —(C₁₋₆ alkyl), unsubstituted —(C₁₋₄ haloalkyl), —OR¹⁰, and —CN;
each R⁶ is independently selected from the group consisting of H, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
R⁷ is selected from the group consisting of H, unsubstituted —(C₁₋₉ alkyl), unsubstituted —(C₃₋₉ alkenyl), unsubstituted —(C₃₋₉ alkynyl), unsubstituted —(C₁₋₉ haloalkyl), —(C₁₋₄ alkylene)C(═O)OR¹¹, —(C₁₋₄ alkylene)C(═O)N(R¹²)₂, —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein;
each R¹⁰ is independently selected from the group consisting of H, unsubstituted —(C₁₋₉ alkyl), unsubstituted —(C₃₋₉ alkenyl), unsubstituted —(C₃₋₉ alkynyl), and unsubstituted —(C₁₋₉ haloalkyl);
R¹¹ is selected from the group consisting of H and unsubstituted —(C₁₋₉ alkyl);
each R¹² is independently selected from the group consisting of H and substituted or unsubstituted —(C₁₋₉ alkyl);
each R¹³ is independently selected from the group consisting of halide, unsubstituted —(C₁₋₉ alkyl), unsubstituted —(C₁₋₉ haloalkyl), and —OR¹⁰;
each R¹⁴ is independently selected from the group consisting of halide, unsubstituted —(C₁₋₉ alkyl), unsubstituted —(C₁₋₉ haloalkyl), and —OR¹⁰;
each R¹⁵ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁶ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁷ is independently selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R¹⁸ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR¹⁰, —(C₁₋₄ alkylene)ₚheteroaryl optionally substituted with 1-4 R²², —(C₁₋₄ alkylene)ₚaryl optionally substituted with 1-5 R²³, and —CN; wherein each —(C₁₋₄ alkylene) is, independently, optionally substituted with one or more substituents as defined herein;
R¹⁹ is selected from the group consisting of H, halide, unsubstituted —(C₁₋₉ alkyl), and unsubstituted —(C₁₋₆ haloalkyl);
R²⁰ is selected from the group consisting of H, halide, unsubstituted —(C₁₋₉ alkyl), and unsubstituted —(C₁₋₆ haloalkyl);
R²¹ is selected from the group consisting of H, halide, unsubstituted —(C₁₋₉ alkyl), and unsubstituted —(C₁₋₆ haloalkyl);
each R²² is independently selected from the group consisting of halide, unsubstituted —(C₁₋₉ alkyl), unsubstituted —(C₁₋₉ haloalkyl), and —CN;
each R²³ is independently selected from the group consisting of halide, unsubstituted —(C₁₋₉ alkyl), unsubstituted —(C₁₋₉ haloalkyl), and —CN;
A¹ is selected from the group consisting of —C(R¹⁵)₂—, O, and S;
A² is selected from the group consisting of O and S;
each A⁵ is independently selected from the group consisting of —C(R¹⁶)₂—, —N(R¹⁰)—, O, and S;
with the proviso that two A⁵ are not both N, O or S;
A⁶ is selected from the group consisting of —C(R¹⁶)₂—, —N(R¹⁰)—, O, and S;
with the proviso that A⁵ and A⁶ cannot both be C(R¹⁶)₂;
m is 1 or 2;
each p independently is 0 or 1; and
with the proviso that Formula II is not a structure selected from the group consisting of:

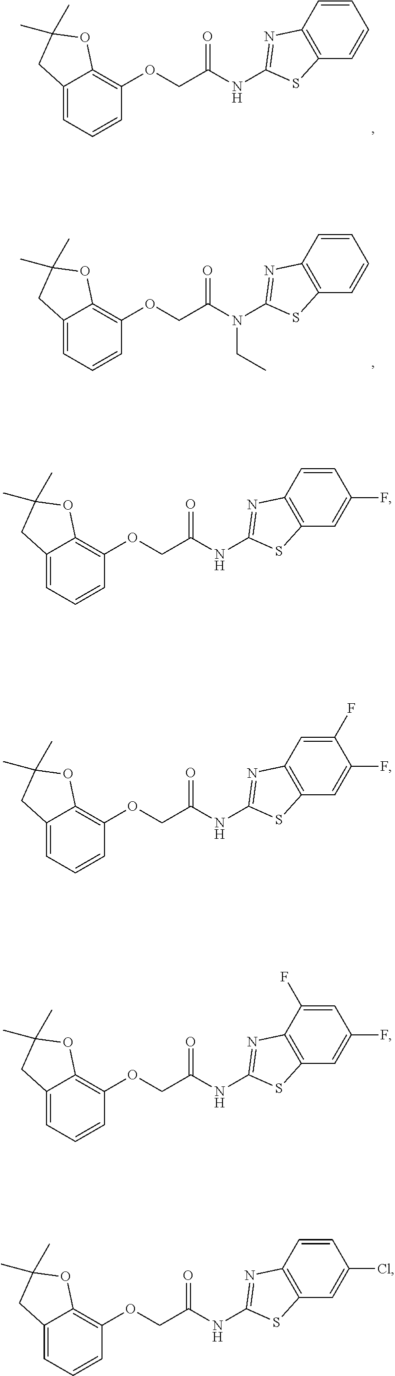 [SMILES: CC1(C)Cc2cccc(OCC(=O)Nc3nc4ccccc4s3)c2O1 ; CCN(C(=O)COc1cccc2c1OC(C)(C)C2)c1nc2ccccc2s1 ; CC1(C)Cc2cccc(OCC(=O)Nc3nc4ccc(F)cc4s3)c2O1 ; CC1(C)Cc2cccc(OCC(=O)Nc3nc4cc(F)c(F)cc4s3)c2O1 ; CC1(C)Cc2cccc(OCC(=O)Nc3nc4c(F)cc(F)cc4s3)c2O1 ; CC1(C)Cc2cccc(OCC(=O)Nc3nc4ccc(Cl)cc4s3)c2O1]

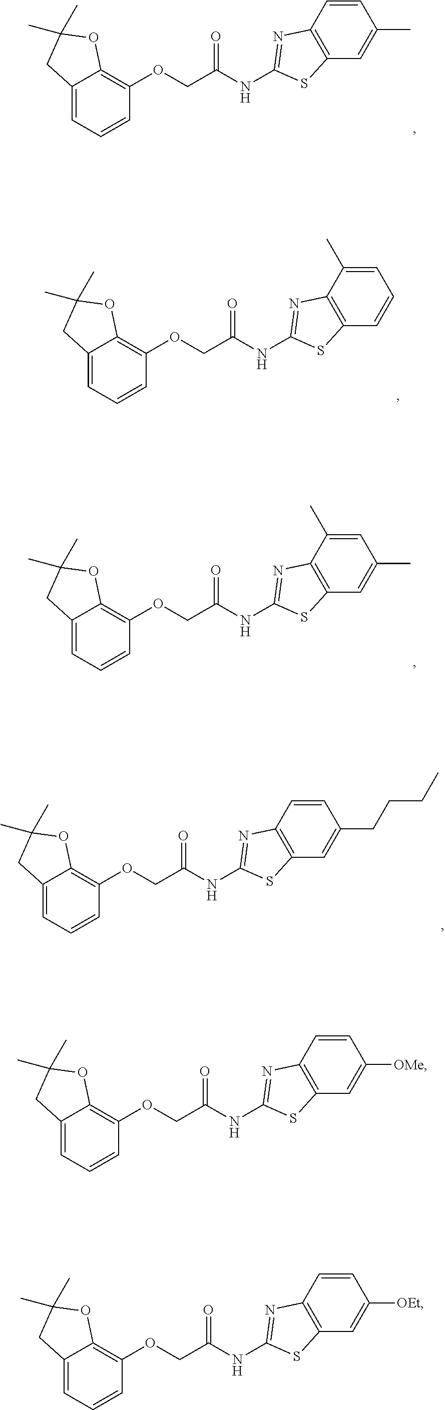

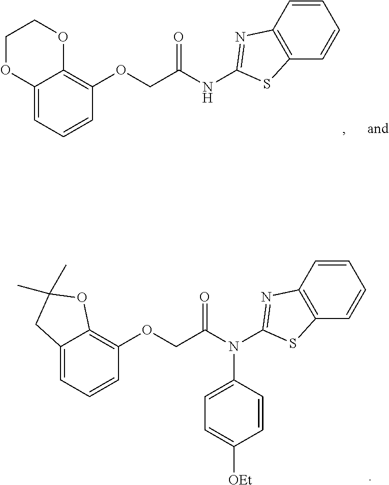 [SMILES: O=C(COc1cccc2c1OCCO2)Nc1nc2ccccc2s1 ; CCOc1ccc(N(C(=O)COc2cccc3c2OC(C)(C)C3)c2nc3ccccc3s2)cc1]

In some embodiments, provided herein is a compound, or a pharmaceutically acceptable salt thereof, of Formula II:

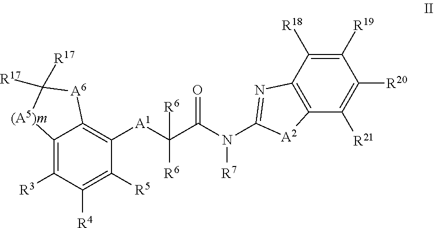

wherein:
R³, R⁴, and R⁵ are independently selected from the group consisting of H, halide, unsubstituted —(C₁₋₆ alkyl), unsubstituted —(C₁₋₄ haloalkyl), —OR¹⁰, and —CN;
each R⁶ is independently selected from the group consisting of H, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
R⁷ is selected from the group consisting of H, unsubstituted —(C₁₋₉ alkyl), unsubstituted —(C₃₋₉ alkenyl), unsubstituted —(C₃₋₉ alkynyl), unsubstituted —(C₁₋₉ haloalkyl), —(C₁₋₄ alkylene)C(═O)OR¹¹, —(C₁₋₄ alkylene)C(═O)N(R¹²)₂, —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein;
each R¹⁰ is independently selected from the group consisting of H, unsubstituted —(C₁₋₉ alkyl), unsubstituted —(C₃₋₉ alkenyl), unsubstituted —(C₃₋₉ alkynyl), and unsubstituted —(C₁₋₉ haloalkyl);
R¹¹ is selected from the group consisting of H and unsubstituted —(C₁₋₉ alkyl);
each R¹² is independently selected from the group consisting of H and substituted or unsubstituted —(C₁₋₉ alkyl);
each R¹³ is independently selected from the group consisting of halide, unsubstituted —(C₁₋₉ alkyl), unsubstituted —(C₁₋₉ haloalkyl), and —OR¹⁰;
each R¹⁴ is independently selected from the group consisting of halide, unsubstituted —(C₁₋₉ alkyl), unsubstituted —(C₁₋₉ haloalkyl), and —OR¹⁰;
each R¹⁵ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁶ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁷ is independently selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R¹⁸ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR¹⁰, —(C₁₋₄ alkylene)ₚheteroaryl optionally substituted with 1-4 R²², —(C₁₋₄ alkylene)ₚaryl optionally substituted with 1-5 R²³, and —CN; wherein each —(C₁₋₄ alkylene) is, independently, optionally substituted with one or more substituents as defined herein;
R¹⁹ is selected from the group consisting of H, halide, unsubstituted —(C₁₋₉ alkyl), and unsubstituted —(C₁₋₆ haloalkyl);
R²⁰ is selected from the group consisting of H, halide, unsubstituted —(C₁₋₉ alkyl), and unsubstituted —(C₁₋₆ haloalkyl);
R²¹ is selected from the group consisting of H, halide, unsubstituted —(C₁₋₉ alkyl), and unsubstituted —(C₁₋₆ haloalkyl);
each R²² is independently selected from the group consisting of halide, unsubstituted —(C₁₋₉ alkyl), unsubstituted —(C₁₋₉ haloalkyl), and —CN;
each R²³ is independently selected from the group consisting of halide, unsubstituted —(C₁₋₉ alkyl), unsubstituted —(C₁₋₉ haloalkyl), and —CN;
A¹ is selected from the group consisting of —C(R¹⁵)₂—, O, and S;
A² is selected from the group consisting of O and S;
each A⁵ is independently selected from the group consisting of —C(R¹⁶)₂—, —N(R¹⁰)—, O, and S;
with the proviso that two A⁵ are not both N, O or S;
A⁶ is selected from the group consisting of —C(R¹⁶)₂—, —N(R¹⁰)—, O, and S;
with the proviso that A⁵ and A⁶ cannot both be C(R¹⁶)₂;
m is 1 or 2; and
each p independently is 0 or 1.

For example, provided herein is a compound, or a pharmaceutically acceptable salt thereof, of Formula II:

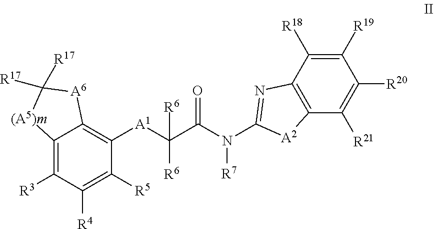

wherein:
R³, R⁴, and R⁵ are independently selected from the group consisting of H, halide, unsubstituted —(C₁₋₆ alkyl), unsubstituted —(C₁₋₄ haloalkyl), —OR¹⁰, and —CN;
each R⁶ is independently selected from the group consisting of H, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
R⁷ is selected from the group consisting of H, unsubstituted —(C₁₋₉ alkyl), unsubstituted —(C₃₋₉ alkenyl), unsubstituted —(C₃₋₉ alkynyl), unsubstituted —(C₁₋₉ haloalkyl), —(C₁₋₄ alkylene)C(═O)OR¹¹, —(C₁₋₄ alkylene)C(═O)N(R¹²)₂, —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein;
each R¹⁰ is independently selected from the group consisting of H, unsubstituted —(C₁₋₉ alkyl), unsubstituted —(C₃₋₉ alkenyl), unsubstituted —(C₃₋₉ alkynyl), and unsubstituted —(C₁₋₉ haloalkyl);
R¹¹ is selected from the group consisting of H and unsubstituted —(C₁₋₉ alkyl);
each R¹² is independently selected from the group consisting of H and substituted or unsubstituted —(C₁₋₉ alkyl);
each R¹³ is independently selected from the group consisting of halide, unsubstituted —(C₁₋₉ alkyl), unsubstituted —(C₁₋₉ haloalkyl), and —OR¹⁰;
each R¹⁴ is independently selected from the group consisting of halide, unsubstituted —(C₁₋₉ alkyl), unsubstituted —(C₁₋₉ haloalkyl), and —OR¹⁰;
each R¹⁵ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁶ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁷ is independently selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R¹⁸ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR¹⁰, —(C₁₋₄ alkylene)ₚheteroaryl optionally substituted with 1-4 R²², —(C₁₋₄ alkylene)ₚaryl optionally substituted with 1-5 R²³, and —CN; wherein each —(C₁₋₄ alkylene) is, independently, optionally substituted with one or more substituents as defined herein;
with the proviso that when A¹ is O or S, both R⁶ are H and R⁷ is selected from the group consisting of H, unsubstituted —(C₁₋₄ alkyl), and -aryl optionally substituted with —OR¹⁰; then R¹⁸ is not selected from the group consisting of H, halide, and unsubstituted —(C₁₋₄ alkyl);
R¹⁹ is selected from the group consisting of H, halide, unsubstituted —(C₁₋₉ alkyl), and unsubstituted —(C₁₋₆ haloalkyl);
R²⁰ is selected from the group consisting of H, halide, unsubstituted —(C₁₋₉ alkyl), and unsubstituted —(C₁₋₆ haloalkyl);
R²¹ is selected from the group consisting of H, halide, unsubstituted —(C₁₋₉ alkyl), and unsubstituted —(C₁₋₆ haloalkyl);
each R²² is independently selected from the group consisting of halide, unsubstituted —(C₁₋₉ alkyl), unsubstituted —(C₁₋₉ haloalkyl), and —CN;
each R²³ is independently selected from the group consisting of halide, unsubstituted —(C₁₋₉ alkyl), unsubstituted —(C₁₋₉ haloalkyl), and —CN;
A¹ is selected from the group consisting of —C(R¹⁵)₂—, O, and S;
A² is selected from the group consisting of O and S;
each A⁵ is independently selected from the group consisting of —C(R¹⁶)₂—, —N(R¹⁰)—, O, and S;
with the proviso that two A⁵ are not both N, O or S;
A⁶ is selected from the group consisting of —C(R¹⁶)₂—, —N(R¹⁰)—, O, and S;
with the proviso that A⁵ and A⁶ cannot both be C(R¹⁶)₂;
m is 1 or 2; and
each p independently is 0 or 1.

Some embodiments of the present disclosure include compounds of Formula III:

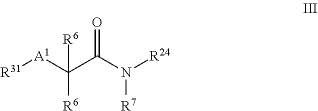

or enantiomers, diastereomers, tautomers, prodrugs, and pharmaceutically acceptable salts thereof.

In some embodiments of Formula III, R²⁴ is selected from the group consisting of a 6-membered heteroaryl optionally substituted with 1-4 R²⁵,

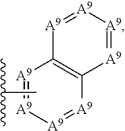

wherein 1-4 (e.g., 1-3. 1-2. 1) A⁹ are N; and

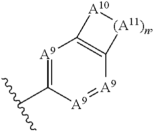

wherein 1-2 (e.g., 1) A⁹ are N.

In some embodiments of Formula III, R²⁴ is a 6-membered heteroaryl optionally substituted with 1-4 R²⁵.

In some embodiments of Formula III, R²⁴ is a 6-membered heteroaryl optionally substituted with 1-4 R²⁵; wherein the 6-membered heteroaryl is selected from the group consisting of

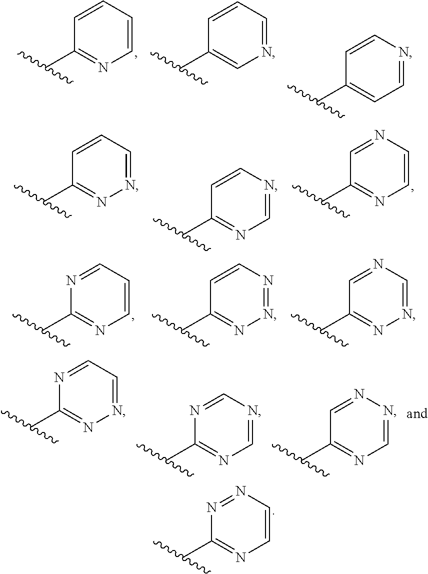

In some embodiments of Formula III, R²⁴ is selected from the group consisting of

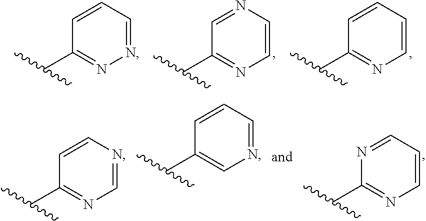

each optionally substituted with 1-2 R²⁵.

In some embodiments of Formula III, R²⁴ is

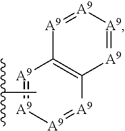

wherein 1-4 (e.g., 1-3. 1-2. 1) A⁹ are N, wherein when A⁹ is carbon, the carbon is optionally substituted with R²⁶.

In some embodiments of Formula III, R²⁴ is

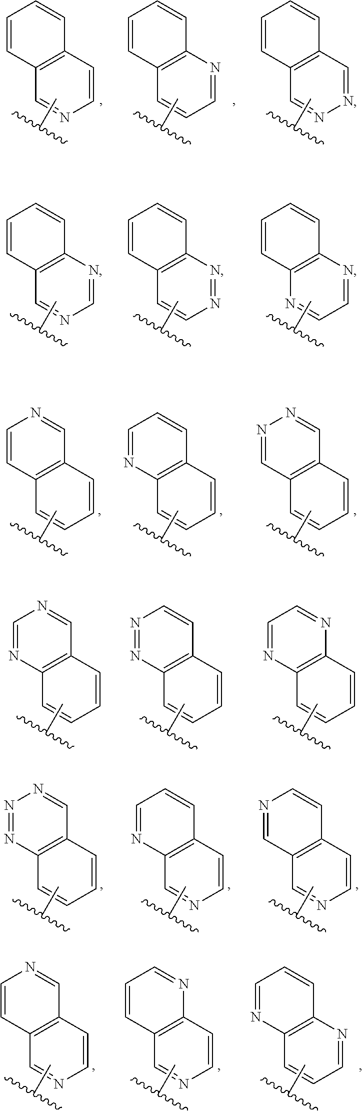

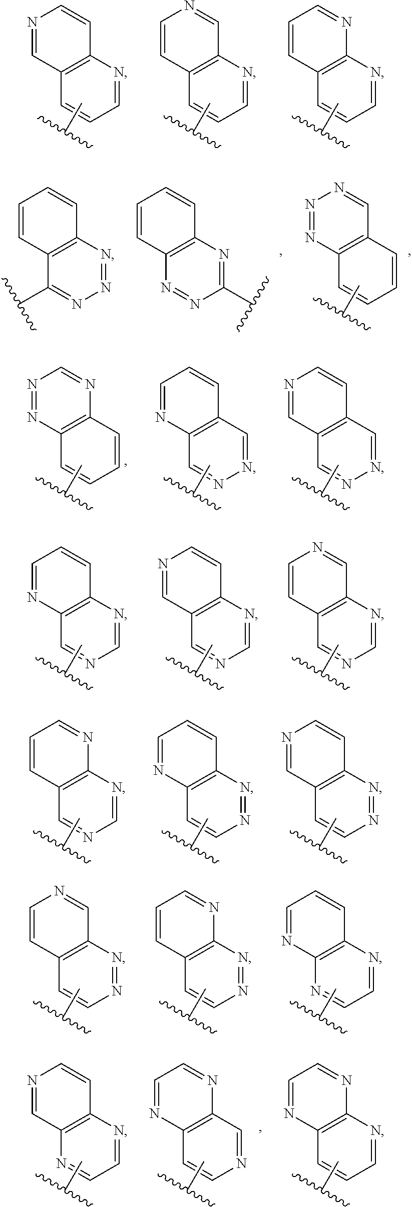

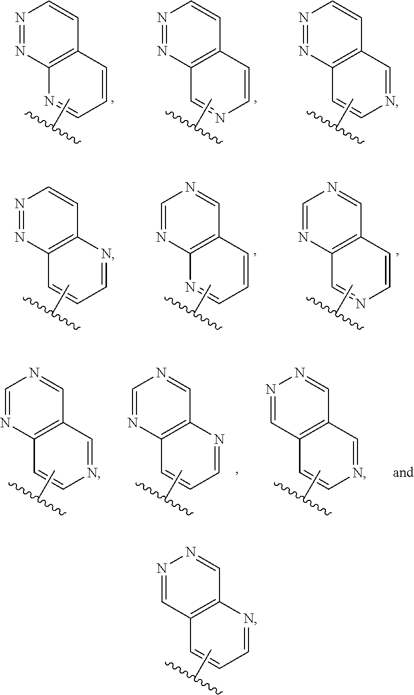

wherein the carbon atoms are optionally substituted with R²⁶.

In some embodiments of Formula III, R²⁴ is selected from the group consisting of

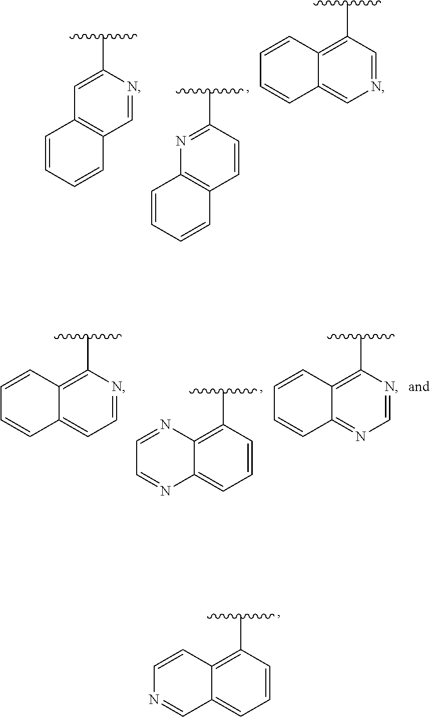

wherein the carbon atoms are optionally substituted with R²⁶.

In some embodiments of Formula III, R²⁴ is

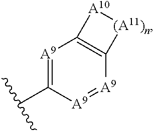

wherein 1-2 (e.g., 1) A⁹ are N, wherein when A⁹ is carbon, the carbon is optionally substituted with R²⁶.

In some embodiments of Formula III, R²⁴ is selected from the group consisting of

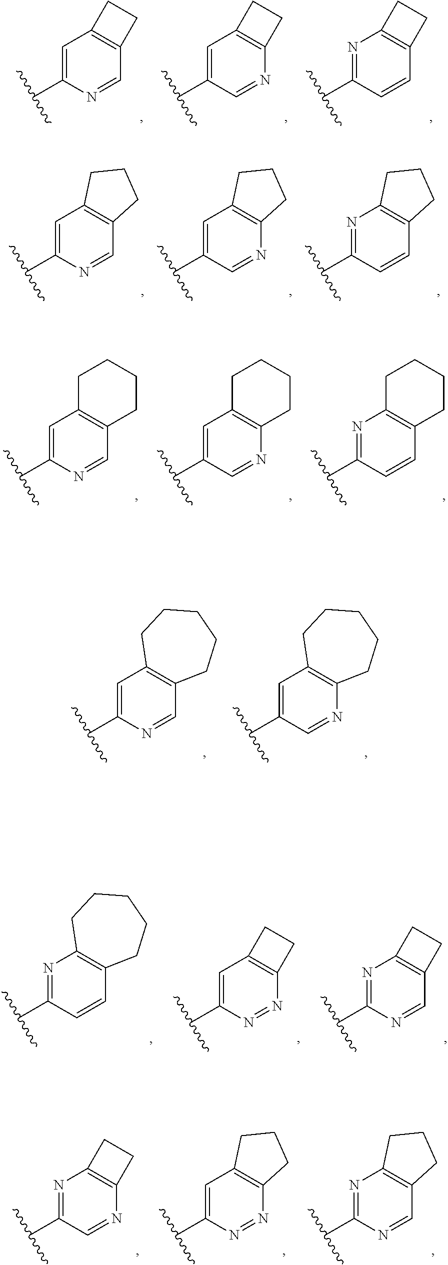

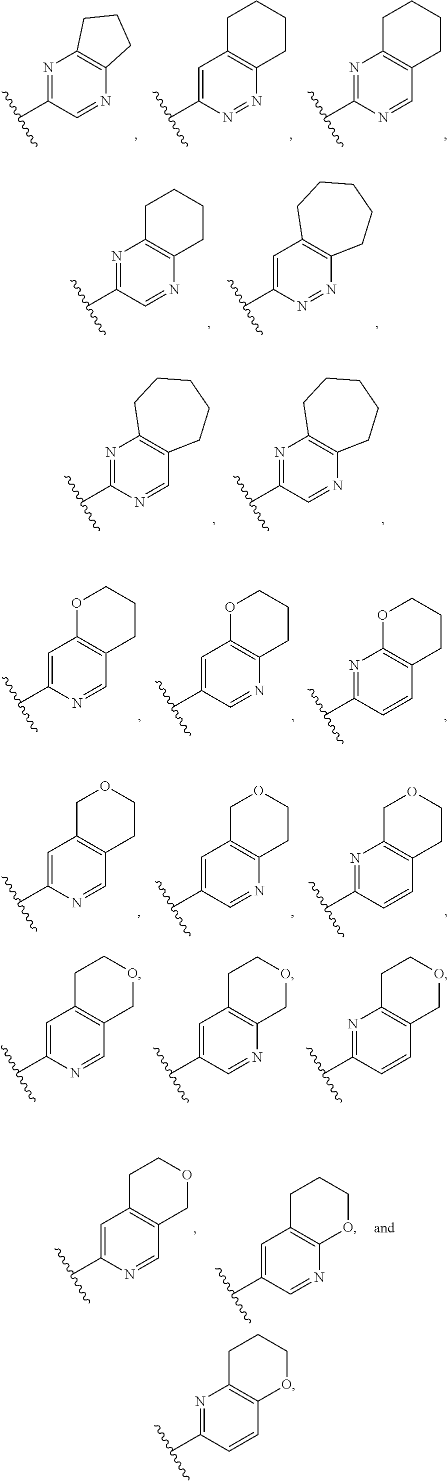

wherein the aromatic carbon atoms are optionally substituted with R²⁶ and the aliphatic carbon atoms are optionally substituted with R²⁷.

In some embodiments of Formula III, each R²⁵ is independently selected from the group consisting of halide (e.g., F, Cl, Br, I), —CN, —OR²⁸, unsubstituted —(C₁₋₆ alkyl) (e.g., C₁₋₅, C₁₋₄, C₁₋₃, C₁₋₂, C₁), unsubstituted —(C₂₋₆ alkenyl) (e.g., C₂₋₅, C₂₋₄, C₂₋₃, C₂), unsubstituted —(C₂₋₆ alkynyl) (e.g., C₂₋₅, C₂₋₄, C₂₋₃, C₂), unsubstituted —(C₁₋₆ haloalkyl) (e.g., C₁₋₅, C₁₋₄, C₁₋₃, C₁₋₂, C₁), and carbocyclyl optionally substituted with 1-10 (e.g., 1-9, 1-8, 1-7, 1-6, 1-5, 1-4, 1-3. 1-2. 1) R²⁹.

In some embodiments of Formula III, each R²⁵ is independently selected from the group consisting of F, Cl, Br, —CN, —O(C₁₋₄ alkyl) (e.g., C₁₋₃, C₁₋₂, C₁), unsubstituted —(C₁₋₄ alkyl) (e.g., C₁₋₃, C₁₋₂, C₁), unsubstituted —(C₂₋₄ alkenyl) (e.g., C₂₋₃, C₂), unsubstituted —(C₂₋₄ alkynyl) (e.g., C₂₋₃, C₂), unsubstituted —(C₁₋₄ haloalkyl) (e.g., C₁₋₃, C₁₋₂, C₁), and carbocyclyl optionally substituted with 1-4 (e.g., 1-3. 1-2. 1) R²⁹.

In some embodiments of Formula III, each R²⁵ is independently selected from the group consisting of F, Cl, —CN, —O(C₁₋₂ alkyl) (e.g., Me), unsubstituted —(C₁₋₂ alkyl) (e.g., Me), unsubstituted —(C₂ alkenyl), unsubstituted —(C₂ alkynyl), unsubstituted —(C₁₋₂ haloalkyl) (e.g., C₁), and carbocyclyl optionally substituted with 1-2 (e.g., 1) R²⁹.

In some embodiments of Formula III, each R²⁵ is independently selected from the group consisting of F, Cl, —CN, —OMe, Me, —CF₃, and an unsubstituted C₃₋₄ carbocyclyl.

In some embodiments of Formula III, each R²⁶ is independently selected from the group consisting of H, halide (e.g., F, Cl, Br, I), —CN, —OR²⁸, unsubstituted —(C₁₋₆ alkyl) (e.g., C₁₋₅, C₁₋₄, C₁₋₃, C₁₋₂, C₁), unsubstituted —(C₂₋₆ alkenyl) (e.g., C₂₋₅, C₂₋₄, C₂₋₃, C₂), unsubstituted —(C₂₋₆ alkynyl) (e.g., C₂₋₅, C₂₋₄, C₂₋₃, C₂), unsubstituted —(C₁₋₆ haloalkyl) (e.g., C₁₋₅, C₁₋₄, C₁₋₃, C₁₋₂, C₁), and carbocyclyl optionally substituted with 1-10 (e.g., 1-9, 1-8, 1-7, 1-6, 1-5, 1-4, 1-3. 1-2. 1) R²⁹.

In some embodiments of Formula III, each R²⁶ is independently selected from the group consisting of F, Cl, Br, —CN, —O(C₁₋₄ alkyl) (e.g., C₁₋₃, C₁₋₂, C₁), unsubstituted —(C₁₋₄ alkyl) (e.g., C₁₋₃, C₁₋₂, C₁), unsubstituted —(C₂₋₄ alkenyl) (e.g., C₂₋₃, C₂), unsubstituted —(C₂₋₄ alkynyl) (e.g., C₂₋₃, C₂), unsubstituted —(C₁₋₄ haloalkyl) (e.g., C₁₋₃, C₁₋₂, C₁), and carbocyclyl optionally substituted with 1-4 (e.g., 1-3. 1-2. 1) R²⁹.

In some embodiments of Formula III, each R²⁶ is independently selected from the group consisting of F, Cl, —CN, —O(C₁₋₂ alkyl) (e.g., Me), unsubstituted —(C₁₋₂ alkyl) (e.g., Me), unsubstituted —(C₂ alkenyl), unsubstituted —(C₂ alkynyl), unsubstituted —(C₁₋₂ haloalkyl) (e.g., C₁), and carbocyclyl optionally substituted with 1-2 (e.g., 1) R²⁹.

In some embodiments of Formula III, each R²⁶ is independently selected from the group consisting of F, Cl, —CN, —OMe, Me, —CF₃, and an unsubstituted C₃₋₄ carbocyclyl.

In some embodiments of Formula III, each R²⁷ is independently selected from the group consisting of H, halide (e.g., F, Cl, Br, I), —CN, —OR²⁸, unsubstituted —(C₁₋₆ alkyl) (e.g., C₁₋₅, C₁₋₄, C₁₋₃, C₁₋₂, C₁), unsubstituted —(C₂₋₆ alkenyl) (e.g., C₂₋₈, C₂₋₄, C₂₋₃, C₂), unsubstituted —(C₂₋₆ alkynyl) (e.g., C₂₋₅, C₂₋₄, C₂₋₃, C₂), unsubstituted —(C₁₋₆ haloalkyl) (e.g., C₁₋₅, C₁₋₄, C₁₋₃, C₁₋₂, C₁), and carbocyclyl optionally substituted with 1-10 (e.g., 1-9, 1-8, 1-7, 1-6, 1-5, 1-4, 1-3. 1-2. 1) R²⁹.

In some embodiments of Formula III, each R²⁷ is independently selected from the group consisting of F, Cl, Br, —CN, —O(C₁₋₄ alkyl) (e.g., C₁₋₃, C₁₋₂, C₁), unsubstituted —(C₁₋₄ alkyl) (e.g., C₁₋₃, C₁₋₂, C₁), unsubstituted —(C₂₋₄ alkenyl) (e.g., C₂₋₃, C₂), unsubstituted —(C₂₋₄ alkynyl) (e.g., C₂₋₃, C₂), unsubstituted —(C₁₋₄ haloalkyl) (e.g., C₁₋₃, C₁₋₂, C₁), and carbocyclyl optionally substituted with 1-4 (e.g., 1-3. 1-2. 1) R²⁹.

In some embodiments of Formula III, each R²⁷ is independently selected from the group consisting of F, Cl, —CN, —O(C₁₋₂ alkyl) (e.g., Me), unsubstituted —(C₁₋₂ alkyl) (e.g., Me), unsubstituted —(C₂ alkenyl), unsubstituted —(C₂ alkynyl), unsubstituted —(C₁₋₂ haloalkyl) (e.g., C₁), and carbocyclyl optionally substituted with 1-2 (e.g., 1) R²⁹.

In some embodiments of Formula III, each R²⁷ is independently selected from the group consisting of F, Cl, —CN, —OMe, Me, —CF₃, and an unsubstituted C₃₋₄ carbocyclyl.

In some embodiments of Formula III, each R²⁸ is independently selected from the group consisting of unsubstituted —(C₁₋₉ alkyl) (e.g., C₁₋₈, C₁₋₇, C₁₋₆, C₁₋₈, C₁₋₄, C₁₋₃, C₁₋₂, C₁), unsubstituted —(C₃₋₉ alkenyl) (e.g., C₃₋₈, C₃₋₇, C₃₋₆, C₃₋₅, C₃₋₄, C₃), unsubstituted —(C₃₋₉ alkynyl) (e.g., C₃₋₈, C₃₋₇, C₃₋₆, C₃₋₅, C₃₋₄, C₃), and unsubstituted —(C₁₋₉ haloalkyl) (e.g., C₁₋₈, C₁₋₇, C₁₋₆, C₁₋₈, C₁₋₄, C₁₋₃, C₁₋₂, C₁).

In some embodiments of Formula III, each R²⁸ is independently selected from the group consisting of unsubstituted —(C₁₋₄ alkyl) (e.g., C₁₋₃, C₁₋₂, C₁), unsubstituted —(C₃₋₄ alkenyl) (e.g., C₃), unsubstituted —(C₃₋₄ alkynyl) (e.g., C₃), and unsubstituted —(C₁₋₄ haloalkyl) (e.g., C₁₋₃, C₁₋₂, C₁).

In some embodiments of Formula III, each R²⁸ is independently selected from the group consisting of unsubstituted —(C₁₋₂ alkyl) (e.g., Me) and unsubstituted —(C₁₋₂ haloalkyl) (e.g., C₁).

In some embodiments of Formula III, each R²⁸ is independently selected from the group consisting of Me and —CF₃.

In some embodiments of Formula III, each R²⁹ is independently selected from the group consisting of H, halide (e.g., F, Cl, Br, I), unsubstituted —(C₁₋₆ alkyl) (e.g., C₁₋₈, C₁₋₄, C₁₋₃, C₁₋₂, C₁), unsubstituted —(C₂₋₆ alkenyl) (e.g., C₂₋₈, C₂₋₄, C₂₋₃, C₂), unsubstituted —(C₂₋₆ alkynyl) (e.g., C₂₋₅, C₂₋₄, C₂₋₃, C₂), unsubstituted —(C₁₋₆ haloalkyl) (e.g., C₁₋₅, C₁₋₄, C₁₋₃, C₁₋₂, C₁).

In some embodiments of Formula III, each R²⁹ is independently selected from the group consisting of H, F, Cl, Br, unsubstituted —(C₁₋₄ alkyl) (e.g., C₁₋₃, C₁₋₂, C₁), unsubstituted —(C₂₋₄ alkenyl) (e.g., C₂₋₃, C₂), unsubstituted —(C₂₋₄ alkynyl) (e.g., C₂₋₃, C₂), and unsubstituted —(C₁₋₄ haloalkyl) (e.g., C₁₋₃, C₁₋₂, C₁).

In some embodiments of Formula III, each R²⁹ is independently selected from the group consisting of H, F, Cl, unsubstituted —(C₁₋₂ alkyl) (e.g., Me), unsubstituted —(C₂ alkenyl), unsubstituted —(C₂ alkynyl), and unsubstituted —(C₁₋₂ haloalkyl) (e.g., C₁).

In some embodiments of Formula III, each R²⁹ is independently selected from the group consisting of H, F, Cl, Me, and —CF₃.

In some embodiments of Formula III, each A⁹ is independently selected from the group consisting of N and —C(R²⁶)—.

In some embodiments of Formula III, each A⁹ is independently selected from the group consisting of N and —CH—.

In some embodiments of Formula III, A¹⁰ is selected from the group consisting of —C(R²⁷)₂—, O, and S.

In some embodiments of Formula III, A¹⁰ is —C(R²⁷)₂—.

In some embodiments of Formula III, A¹⁰ is —CH₂—.

In some embodiments of Formula III, A¹⁰ is O.

In some embodiments of Formula III, A¹⁰ is S.

In some embodiments of Formula III, each A¹¹ is independently selected from the group consisting of —C(R²⁷)₂—, O, and S.

In some embodiments of Formula III, each A¹¹ is independently selected from the group consisting of —CH₂— and S.

In some embodiments of Formula III, each A¹¹ is independently selected from the group consisting of —CH₂— and O.

In some embodiments of Formula III, each A¹¹ is —C(R²⁷)₂—.

In some embodiments of Formula III, each A¹¹ is —CH₂—.

In some embodiments of Formula III, there is the proviso that no more than two A¹⁰ or A¹¹ are O or S and that two O or S are never adjacent.

In some embodiments, provided herein is a compound, or a pharmaceutically acceptable salt thereof, of Formula III:

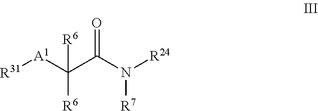

wherein:
R³, R⁴, and R⁵ are independently selected from the group consisting of H, halide, unsubstituted —(C₁₋₆ alkyl), unsubstituted —(C₁₋₄ haloalkyl), —OR¹⁰, and —CN;
each R⁶ is independently selected from the group consisting of H, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein;
R⁷ is selected from the group consisting of H, unsubstituted —(C₁₋₉ alkyl), unsubstituted —(C₃₋₉ alkenyl), unsubstituted —(C₃₋₉ alkynyl), unsubstituted —(C₁₋₉ haloalkyl), —(C₁₋₄ alkylene)C(═O)OR¹¹, —(C₁₋₄ alkylene)C(═O)N(R¹²)₂, —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein;
each R¹⁰ is independently selected from the group consisting of H, unsubstituted —(C₁₋₉ alkyl), unsubstituted —(C₃₋₉ alkenyl), unsubstituted —(C₃₋₉ alkynyl), and unsubstituted —(C₁₋₉ haloalkyl);
R¹¹ is selected from the group consisting of H and unsubstituted —(C₁₋₉ alkyl);
each R¹² is independently selected from the group consisting of H and substituted or unsubstituted —(C₁₋₉ alkyl);
each R¹³ is independently selected from the group consisting of halide, unsubstituted —(C₁₋₉ alkyl), unsubstituted —(C₁₋₉ haloalkyl), and —OR¹⁰;
each R¹⁴ is independently selected from the group consisting of halide, unsubstituted —(C₁₋₉ alkyl), unsubstituted —(C₁₋₉ haloalkyl), and —OR¹⁰;
each R¹⁵ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁶ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁷ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R²⁴ is selected from the group consisting of a 6-membered heteroaryl optionally substituted with 1-4 R²⁵,

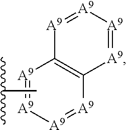

wherein 1-4 A⁹ are N; and

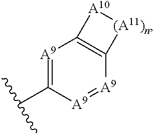

wherein 1-2 A⁹ are N;
each R²⁵ is independently selected from the group consisting of halide, —CN, —OR²⁸, unsubstituted —(C₁₋₆ alkyl), unsubstituted —(C₂₋₆ alkenyl), unsubstituted —(C₂₋₆ alkynyl), unsubstituted —(C₁₋₆ haloalkyl), and carbocyclyl optionally substituted with 1-10 R²⁹;
each R²⁶ is independently selected from the group consisting of H, halide, —CN, —OR²⁸, unsubstituted —(C₁₋₆ alkyl), unsubstituted —(C₂₋₆ alkenyl), unsubstituted —(C₂₋₆ alkynyl), unsubstituted —(C₁₋₆ haloalkyl), and carbocyclyl optionally substituted with 1-10 R²⁹;
each R²⁷ is independently selected from the group consisting of H, halide, —CN, —OR²⁸, unsubstituted —(C₁₋₆ alkyl), unsubstituted —(C₂₋₆ alkenyl), unsubstituted —(C₂₋₆ alkynyl), unsubstituted —(C₁₋₆ haloalkyl), and carbocyclyl optionally substituted with 1-10 R²⁹;
each R²⁸ is independently selected from the group consisting of unsubstituted —(C₁₋₉ alkyl), unsubstituted —(C₃₋₉ alkenyl), unsubstituted —(C₃₋₉ alkynyl), and unsubstituted —(C₁₋₉ haloalkyl);
each R²⁹ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
R³¹ is selected from the group consisting of H and unsubstituted —(C₁₋₉ alkyl);
R³¹ is selected from the group consisting of

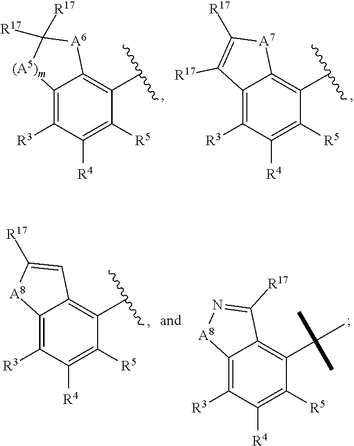

A¹ is selected from the group consisting of —C(R¹⁵)₂—, O, and S;
each A⁵ is independently selected from the group consisting of —C(R¹⁶)₂—, —N(R¹)—, O, and S;
with the proviso that two A⁵ are not both N, O or S;
A⁶ is selected from the group consisting of —C(R¹⁶)₂—, —N(R¹⁰)—, O, and S;
with the proviso that A⁵ and A⁶ cannot both be C(R¹⁶)₂;
A⁷ is selected from the group consisting of —N(R³⁰)—, O and S;
A⁶ is selected from the group consisting of —NH—, O and S;
each A⁹ is independently selected from the group consisting of N and —C(R²⁶)—;
A¹⁰ is selected from the group consisting of —C(R²⁷)₂—, O, and S;
each A¹¹ is independently selected from the group consisting of —C(R²⁷)₂—, O, and S;
with the proviso that no more than two A¹⁰ or A¹¹ are O or S and that two O or S are never adjacent;
n is 1, 2, 3, or 4;
m is 1 or 2; and

each p independently is 0 or 1.

In some embodiments, provided herein is a compound, or a pharmaceutically acceptable salt thereof, of Formula III:

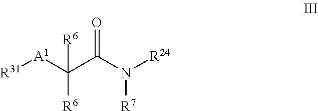

wherein:
R³, R⁴, and R⁵ are independently selected from the group consisting of H, halide, unsubstituted —(C₁₋₆ alkyl), unsubstituted —(C₁₋₄ haloalkyl), —OR¹⁰, and —CN;
each R⁶ is independently selected from the group consisting of H, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein;
R⁷ is selected from the group consisting of H, unsubstituted —(C₁₋₉ alkyl), unsubstituted —(C₃₋₉ alkenyl), unsubstituted —(C₃₋₉ alkynyl), unsubstituted —(C₁₋₉ haloalkyl), —(C₁₋₄ alkylene)C(═O)OR¹¹, —(C₁₋₄ alkylene)C(═O)N(R¹²)₂, —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein;
each R¹⁰ is independently selected from the group consisting of H, unsubstituted —(C₁₋₉ alkyl), unsubstituted —(C₃₋₉ alkenyl), unsubstituted —(C₃₋₉ alkynyl), and unsubstituted —(C₁₋₉ haloalkyl);
R¹¹ is selected from the group consisting of H and unsubstituted —(C₁₋₉ alkyl);
each R¹² is independently selected from the group consisting of H and substituted or unsubstituted —(C₁₋₉ alkyl);
each R¹³ is independently selected from the group consisting of halide, unsubstituted —(C₁₋₉ alkyl), unsubstituted —(C₁₋₉ haloalkyl), and —OR¹⁰;
each R¹⁴ is independently selected from the group consisting of halide, unsubstituted —(C₁₋₉ alkyl), unsubstituted —(C₁₋₉ haloalkyl), and —OR¹⁰;
each R¹⁵ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁶ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁷ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R²⁴ is selected from the group consisting of a 6-membered heteroaryl optionally substituted with 1-4 R²⁵,

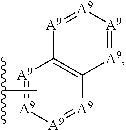

wherein 1-4 A⁹ are N; and

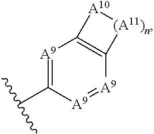

wherein 1-2 A⁹ are N;
each R²⁵ is independently selected from the group consisting of halide, —CN, —OR²⁸, unsubstituted —(C₁₋₆ alkyl), unsubstituted —(C₂₋₆ alkenyl), unsubstituted —(C₂₋₆ alkynyl), unsubstituted —(C₁₋₆ haloalkyl), and carbocyclyl optionally substituted with 1-10 R²⁹;
each R²⁶ is independently selected from the group consisting of H, halide, —CN, —OR²⁸, unsubstituted —(C₁₋₆ alkyl), unsubstituted —(C₂₋₆ alkenyl), unsubstituted —(C₂₋₆ alkynyl), unsubstituted —(C₁₋₆ haloalkyl), and carbocyclyl optionally substituted with 1-10 R²⁹;
each R²⁷ is independently selected from the group consisting of H, halide, —CN, —OR²⁸, unsubstituted —(C₁₋₆ alkyl), unsubstituted —(C₂₋₆ alkenyl), unsubstituted —(C₂₋₆ alkynyl), unsubstituted —(C₁₋₆ haloalkyl), and carbocyclyl optionally substituted with 1-10 R²⁹;
each R²⁸ is independently selected from the group consisting of unsubstituted —(C₁₋₉ alkyl), unsubstituted —(C₃₋₉ alkenyl), unsubstituted —(C₃₋₉ alkynyl), and unsubstituted —(C₁₋₉ haloalkyl);
each R²⁹ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
R³⁰ is selected from the group consisting of H and unsubstituted —(C₁₋₉ alkyl);
R³¹ is selected from the group consisting of

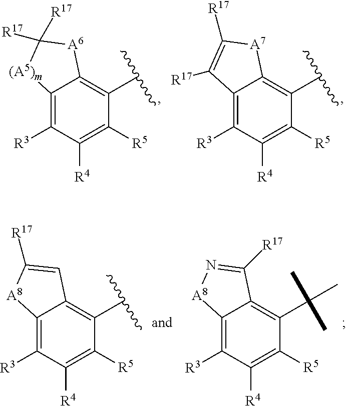

A¹ is selected from the group consisting of —C(R¹⁵)₂—, O, and S;
each A⁵ is independently selected from the group consisting of —C(R¹⁶)₂—, —N(R¹⁰)—, O, and S;
with the proviso that two A⁵ are not both N, O or S;
A⁶ is selected from the group consisting of —C(R¹⁶)₂—, —N(R¹⁰)—, O, and S;
with the proviso that A⁵ and A⁶ cannot both be C(R¹⁶)₂;
A⁷ is selected from the group consisting of —N(R³⁰)—, O and S;
A⁸ is selected from the group consisting of —NH—, O and S;
each A⁹ is independently selected from the group consisting of N and —C(R²⁶)—;
A¹⁰ is selected from the group consisting of —C(R²⁷)₂—, O, and S;
each A¹¹ is independently selected from the group consisting of —C(R²⁷)₂—, O, and S;
with the proviso that no more than two A¹⁰ or A¹¹ are O or S and that two O or S are never adjacent;
n is 1, 2, 3, or 4;
m is 1 or 2;

each p independently is 0 or 1; and

with the proviso that Formula III is not a structure selected from the group consisting of:

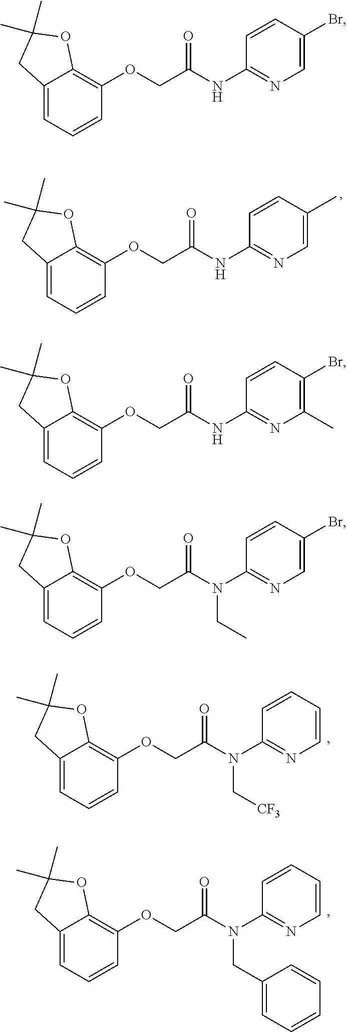

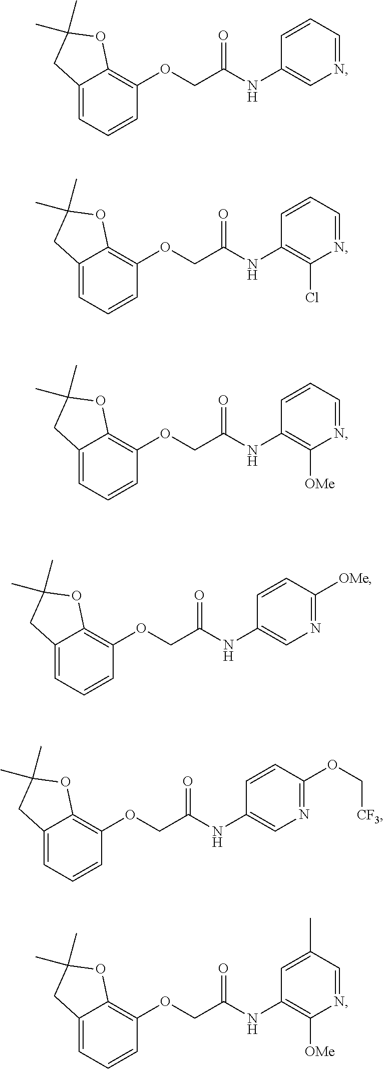 [SMILES: CC1(C)Cc2cccc(OCC(=O)Nc3cccnc3)c2O1 ; CC1(C)Cc2cccc(OCC(=O)Nc3cccnc3Cl)c2O1 ; COc1ncccc1NC(=O)COc1cccc2c1OC(C)(C)C2 ; COc1ccc(NC(=O)COc2cccc3c2OC(C)(C)C3)cn1 ; CC1(C)Cc2cccc(OCC(=O)Nc3ccc(OCC(F)(F)F)nc3)c2O1 ; COc1ncc(C)cc1NC(=O)COc1cccc2c1OC(C)(C)C2]

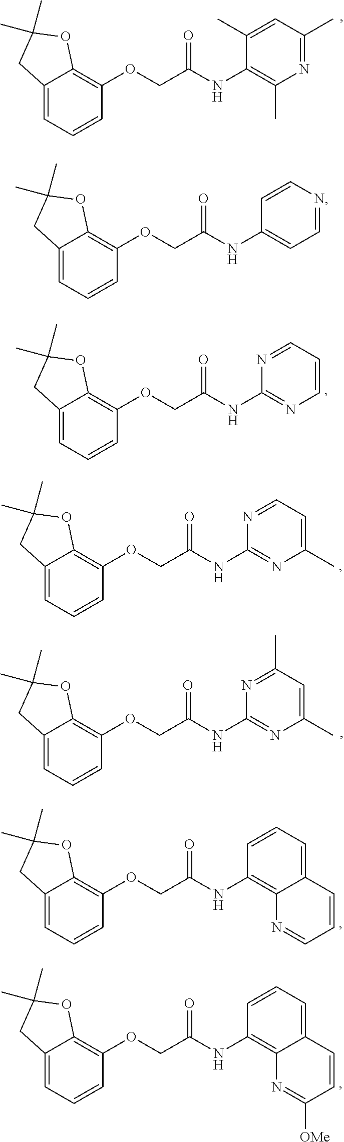

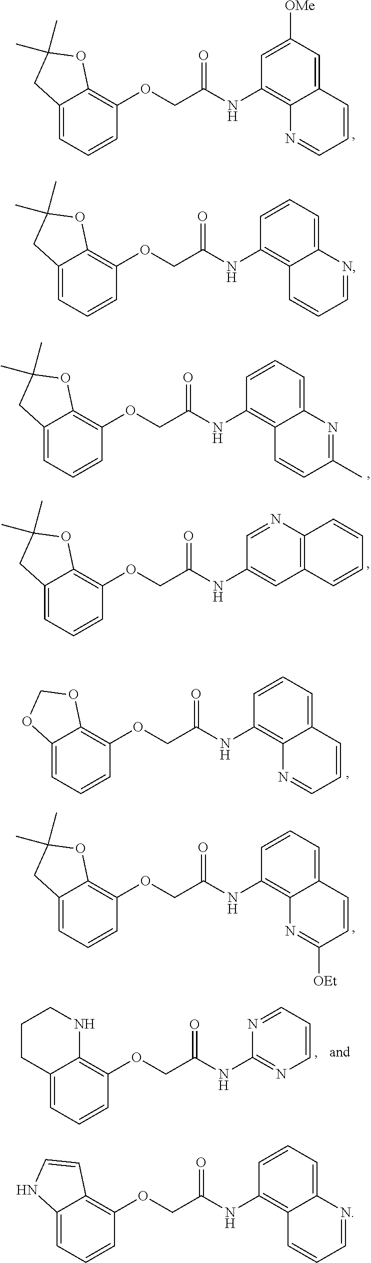

For example, provided herein is a compound, or a pharmaceutically acceptable salt thereof, of Formula III:

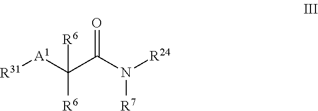

wherein:
R³, R⁴, and R⁵ are independently selected from the group consisting of H, halide, unsubstituted —(C₁₋₆ alkyl), unsubstituted —(C₁₋₄ haloalkyl), —OR¹⁰, and —CN;
each R⁶ is independently selected from the group consisting of H, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein;
R⁷ is selected from the group consisting of H, unsubstituted —(C₁₋₉ alkyl), unsubstituted —(C₃₋₉ alkenyl), unsubstituted —(C₃₋₉ alkynyl), unsubstituted —(C₁₋₉ haloalkyl), —(C₁₋₄ alkylene)C(═O)OR¹¹, —(C₁₋₄ alkylene)C(═O)N(R¹²)₂, —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein;
each R¹⁰ is independently selected from the group consisting of H, unsubstituted —(C₁₋₉ alkyl), unsubstituted —(C₃₋₉ alkenyl), unsubstituted —(C₃₋₉ alkynyl), and unsubstituted —(C₁₋₉ haloalkyl);
R¹¹ is selected from the group consisting of H and unsubstituted —(C₁₋₉ alkyl);
each R¹² is independently selected from the group consisting of H and substituted or unsubstituted —(C₁₋₉ alkyl);
each R¹³ is independently selected from the group consisting of halide, unsubstituted —(C₁₋₉ alkyl), unsubstituted —(C₁₋₉ haloalkyl), and —OR¹⁰;
each R¹⁴ is independently selected from the group consisting of halide, unsubstituted —(C₁₋₉ alkyl), unsubstituted —(C₁₋₉ haloalkyl), and —OR¹⁰;
each R¹⁵ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁶ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁷ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R²⁴ is selected from the group consisting of a 6-membered heteroaryl optionally substituted with 1-4 R²⁵,

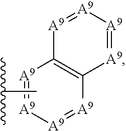

wherein 1-4 A⁹ are N; and

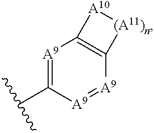

wherein 1-2 A⁹ are N;
each R²⁵ is independently selected from the group consisting of halide, —CN, —OR²⁸, unsubstituted —(C₁₋₆ alkyl), unsubstituted —(C₂₋₆ alkenyl), unsubstituted —(C₂₋₆ alkynyl), unsubstituted —(C₁₋₆ haloalkyl), and carbocyclyl optionally substituted with 1-10 R²⁹;
each R²⁶ is independently selected from the group consisting of H, halide, —CN, —OR²⁸, unsubstituted —(C₁₋₆ alkyl), unsubstituted —(C₂₋₆ alkenyl), unsubstituted —(C₂₋₆ alkynyl), unsubstituted —(C₁₋₆ haloalkyl), and carbocyclyl optionally substituted with 1-10 R²⁹;
each R²⁷ is independently selected from the group consisting of H, halide, —CN, —OR²⁸, unsubstituted —(C₁₋₆ alkyl), unsubstituted —(C₂₋₆ alkenyl), unsubstituted —(C₂₋₆ alkynyl), unsubstituted —(C₁₋₆ haloalkyl), and carbocyclyl optionally substituted with 1-10 R²⁹;
each R²⁸ is independently selected from the group consisting of unsubstituted —(C₁₋₉ alkyl), unsubstituted —(C₃₋₉ alkenyl), unsubstituted —(C₃₋₉ alkynyl), and unsubstituted —(C₁₋₉ haloalkyl);
each R²⁹ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
R³⁰ is selected from the group consisting of H and unsubstituted —(C₁₋₉ alkyl);
R³¹ is selected from the group consisting of

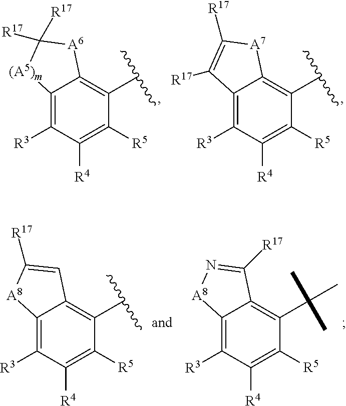

A¹ is selected from the group consisting of —C(R¹⁵)₂—, O, and S;
each A⁵ is independently selected from the group consisting of —C(R¹⁶)₂—, —N(R¹⁰)—, O, and S;
with the proviso that two A⁵ are not both N, O or S;
A⁶ is selected from the group consisting of —C(R¹⁶)₂—, —N(R¹⁰)—, O, and S;
with the proviso that A⁵ and A⁶ cannot both be C(R¹⁶)₂;
A⁷ is selected from the group consisting of —N(R³⁰)—, O and S;
A⁸ is selected from the group consisting of —NH—, O and S;
each A⁹ is independently selected from the group consisting of N and —C(R²⁶)—;
A¹⁰ is selected from the group consisting of —C(R²⁷)₂—, O, and S;
each A¹¹ is independently selected from the group consisting of —C(R²⁷)₂—, O, and S;
with the proviso that no more than two A¹⁰ or A¹¹ are O or S and that two O or S are never adjacent;
n is 1, 2, 3, or 4;
m is 1 or 2; and
each p independently is 0 or 1.

In some embodiments, provided herein is a compound, or a pharmaceutically acceptable salt thereof, of Formula III:

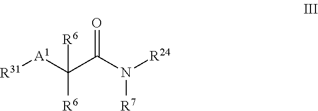

wherein:
R³, R⁴, and R⁵ are independently selected from the group consisting of H, halide, unsubstituted —(C₁₋₆ alkyl), unsubstituted —(C₁₋₄ haloalkyl), —OR¹⁰, and —CN;
each R⁶ is independently selected from the group consisting of H, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein;
R⁷ is selected from the group consisting of H, unsubstituted —(C₁₋₉ alkyl), unsubstituted —(C₃₋₉ alkenyl), unsubstituted —(C₃₋₉ alkynyl), unsubstituted —(C₁₋₉ haloalkyl), —(C₁₋₄ alkylene)C(═O)OR¹¹, —(C₁₋₄ alkylene)C(═O)N(R¹²)₂, —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein;
each R¹⁰ is independently selected from the group consisting of H, unsubstituted —(C₁₋₉ alkyl), unsubstituted —(C₃₋₉ alkenyl), unsubstituted —(C₃₋₉ alkynyl), and unsubstituted —(C₁₋₉ haloalkyl);
R¹¹ is selected from the group consisting of H and unsubstituted —(C₁₋₉ alkyl);
each R¹² is independently selected from the group consisting of H and substituted or unsubstituted —(C₁₋₉ alkyl);
each R¹³ is independently selected from the group consisting of halide, unsubstituted —(C₁₋₉ alkyl), unsubstituted —(C₁₋₉ haloalkyl), and —OR¹⁰;
each R¹⁴ is independently selected from the group consisting of halide, unsubstituted —(C₁₋₉ alkyl), unsubstituted —(C₁₋₉ haloalkyl), and —OR¹⁰;
each R¹⁵ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁶ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁷ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R²⁴ is selected from the group consisting of a 6-membered heteroaryl optionally substituted with 1-4 R²⁵,

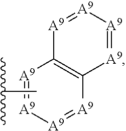

wherein 1-4 A⁹ are N; and

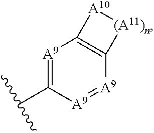

wherein 1-2 A⁹ are N;
with the proviso that when R³¹ is

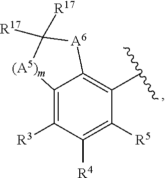

A¹ is O or S, both R⁶ are H, and R⁷ is selected from the group consisting of H, unsubstituted —(C₁₋₃ alkyl), unsubstituted —(C₁₋₃ haloalkyl), —(CH₂)aryl optionally substituted with 1-5 halides, then R²⁴ is not selected from the group consisting of pyridine, 2-pyrimidine, and quinoline;
with the proviso that when R³¹ is

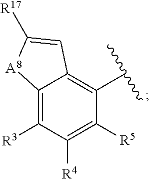

A¹ is O or S, both R⁶ are H, and R⁷ is H, then R²⁴ is not quinoline;
each R²⁵ is independently selected from the group consisting of halide, —CN, —OR²⁸, unsubstituted —(C₁₋₆ alkyl), unsubstituted —(C₂₋₆ alkenyl), unsubstituted —(C₂₋₆ alkynyl), unsubstituted —(C₁₋₆ haloalkyl), and carbocyclyl optionally substituted with 1-10 R²⁹;
each R²⁶ is independently selected from the group consisting of H, halide, —CN, —OR²⁸, unsubstituted —(C₁₋₆ alkyl), unsubstituted —(C₂₋₆ alkenyl), unsubstituted —(C₂₋₆ alkynyl), unsubstituted —(C₁₋₆ haloalkyl), and carbocyclyl optionally substituted with 1-10 R²⁹;
each R²⁷ is independently selected from the group consisting of H, halide, —CN, —OR²⁸, unsubstituted —(C₁₋₆ alkyl), unsubstituted —(C₂₋₆ alkenyl), unsubstituted —(C₂₋₆ alkynyl), unsubstituted —(C₁₋₆ haloalkyl), and carbocyclyl optionally substituted with 1-10 R²⁹;
each R²⁸ is independently selected from the group consisting of unsubstituted —(C₁₋₉ alkyl), unsubstituted —(C₃₋₉ alkenyl), unsubstituted —(C₃₋₉ alkynyl), and unsubstituted —(C₁₋₉ haloalkyl);
each R²⁹ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
R³⁰ is selected from the group consisting of H and unsubstituted —(C₁₋₉ alkyl);
R³¹ is selected from the group consisting of

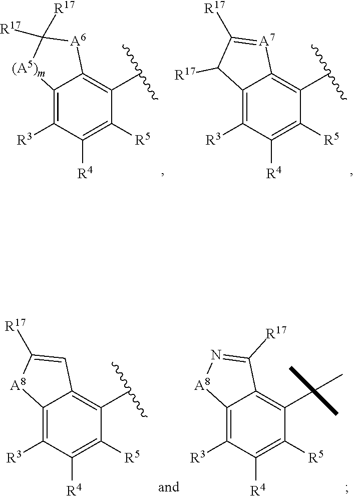

A¹ is selected from the group consisting of —C(R⁵)₂—, O, and S;
each A⁵ is independently selected from the group consisting of —C(R¹⁶)₂—, —N(R¹⁰)—, O, and S;
with the proviso that two A⁵ are not both N, O or S;
A⁶ is selected from the group consisting of —C(R¹⁶)₂—, —N(R¹⁰)—, O, and S;
with the proviso that A⁵ and A⁶ cannot both be C(R¹⁶)₂;
A⁷ is selected from the group consisting of —N(R³⁰)—, O and S;
A⁸ is selected from the group consisting of —NH—, O and S;
each A⁹ is independently selected from the group consisting of N and —C(R²⁶)—;
A¹⁰ is selected from the group consisting of —C(R²⁷)₂—, O, and S;
each A¹¹ is independently selected from the group consisting of —C(R²⁷)₂—, O, and S;
with the proviso that no more than two A¹⁰ or A¹¹ are O or S and that two O or S are never adjacent;
n is 1, 2, 3, or 4;
m is 1 or 2;
each p independently is 0 or 1; and
with the proviso that Formula III is not a structure selected from the group consisting of:

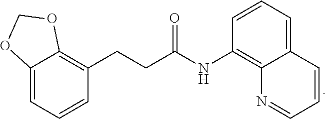 [SMILES: O=C(CCc1cccc2c1OCO2)Nc1cccc2cccnc12]

Some embodiments of the present disclosure include compounds of Formula IV:

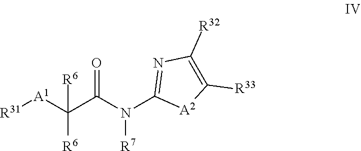

or enantiomers, diastereomers, tautomers, prodrugs, and pharmaceutically acceptable salts thereof.

In some embodiments of Formula IV, R³² and R³³ are independently selected from the group consisting of H, halide (e.g., F, Cl, Br, I), unsubstituted —(C₁₋₉ alkyl) (e.g., C₁₋₈, C₁₋₇, C₁₋₆, C₁₋₅, C₁₋₄, C₁₋₃, C₁₋₂, C₁), unsubstituted —(C₂₋₉ alkenyl) (e.g., C₂₋₈, C₂₋₇, C₂₋₆, C₂₋₅, C₂₋₄, C₂₋₃, C₂), unsubstituted —(C₂₋₉ alkynyl) (e.g., C₂₋₈, C₂₋₇, C₂₋₆, C₂₋₅, C₂₋₄, C₂₋₃, C₂), unsubstituted —(C₁₋₉ haloalkyl) (e.g., C₁₋₈, C₁₋₇, C₁₋₆, C₁₋₅, C₁₋₄, C₁₋₃, C₁₋₂, C₁), -carbocyclyl optionally substituted with 1-10 (e.g., 1-9, 1-8, 1-7, 1-6, 1-5, 1-4, 1-3. 1-2. 1) R³⁴, —C(═O)R³⁵, —C(═O)NHR³⁶, and —CN.

In some embodiments of Formula IV, R³² and R³³ are independently selected from the group consisting of H, F, Cl, Br, unsubstituted —(C₁₋₄ alkyl) (e.g., C₁₋₃, C₁₋₂, C₁), unsubstituted —(C₂₋₄ alkenyl) (e.g., C₂₋₃, C₂), unsubstituted —(C₂₋₄ alkynyl) (e.g., C₂₋₃, C₂), unsubstituted —(C₁₋₄ haloalkyl) (e.g., C₁₋₃, C₁₋₂, C₁), -carbocyclyl optionally substituted with 1-4 (e.g., 1-3. 1-2. 1) R³⁴, —C(═O)R³⁵, —C(═O)NHR³⁶, and —CN.

In some embodiments of Formula IV, R³² and R³³ are independently selected from the group consisting of H, F, Cl, unsubstituted —(C₁₋₂ alkyl) (e.g., Me), unsubstituted —(C₂ alkenyl), unsubstituted —(C₂ alkynyl), unsubstituted —(C₁₋₂ haloalkyl) (e.g., C₁), -carbocyclyl optionally substituted with 1-2 (e.g., 1) R³⁴, —C(═O)R³⁵, —C(═O)NHR³⁶, and —CN.

In some embodiments of Formula IV, R³² and R³³ are independently selected from the group consisting of H, F, Cl, Me, —CF₃, unsubstituted —(C₃₋₄)carbocyclyl, —C(═O)R³⁵, —C(═O)NHR³⁶, and —CN.

In some embodiments of Formula IV, R³² and R³³ are independently selected from the group consisting of H, F, Cl, Me, —CF₃, and —CN.

In some embodiments of Formula IV, each R³⁴ is independently selected from the group consisting of halide (e.g., F, Cl, Br, I), unsubstituted —(C₁₋₉ alkyl) (e.g., C₁₋₈, C₁₋₇, C₁₋₆, C₁₋₅, C₁₋₄, C₁₋₃, C₁₋₂, C₁), unsubstituted —(C₂₋₉ alkenyl) (e.g., C₂₋₈, C₂₋₇, C₂₋₆, C₂₋₅, C₂₋₄, C₂₋₃, C₂), unsubstituted —(C₂₋₉ alkynyl) (e.g., C₂₋₈, C₂₋₇, C₂₋₆, C₂₋₅, C₂₋₄, C₂₋₃, C₂), and unsubstituted —(C₁₋₉ haloalkyl) (e.g., C₁₋₈, C₁₋₇, C₁₋₆, C₁₋₅, C₁₋₄, C₁₋₃, C₁₋₂, C₁).

In some embodiments of Formula IV, R³⁴ is selected from the group consisting of F, Cl, Br, unsubstituted —(C₁₋₄ alkyl) (e.g., C₁₋₃, C₁₋₂, C₁), unsubstituted —(C₂₋₄ alkenyl) (e.g., C₂₋₃, C₂), unsubstituted —(C₂₋₄ alkynyl) (e.g., C₂₋₃, C₂), and unsubstituted —(C₁₋₄ haloalkyl) (e.g., C₁₋₃, C₁₋₂, C₁).

In some embodiments of Formula IV, R³⁴ is selected from the group consisting of H, F, Cl, unsubstituted —(C₁₋₂ alkyl) (e.g., Me), unsubstituted —(C₂ alkenyl), unsubstituted —(C₂ alkynyl), and unsubstituted —(C₁₋₂ haloalkyl) (e.g., C₁).

In some embodiments of Formula IV, R³⁴ is selected from the group consisting of H, F, Cl, Me, and —CF₃.

In some embodiments of Formula IV, R³⁵ is -heterocyclyl optionally substituted with 1-10 (e.g., 1-9, 1-8, 1-7, 1-6, 1-5, 1-4, 1-3. 1-2. 1) R⁴⁰.

In some embodiments of Formula IV, R³⁶ is selected from the group consisting of —(C₁₋₄ alkylene)heterocyclyl optionally substituted with 1-10 (e.g., 1-9, 1-8, 1-7, 1-6, 1-5, 1-4, 1-3. 1-2. 1) R⁴¹ and —(C₁₋₄ alkylene)heteroaryl optionally substituted with 1-10 (e.g., 1-9, 1-8, 1-7, 1-6, 1-5, 1-4, 1-3. 1-2. 1) R⁴²; wherein each —(C₁₋₄ alkylene) is, independently, optionally substituted with one or more substituents as defined herein.

In some embodiments of Formula IV, R³⁶ is selected from the group consisting of —(C₁₋₂ alkylene)heterocyclyl optionally substituted with 1-4 (e.g., 1-3. 1-2. 1) R⁴¹ and —(C₁₋₂ alkylene)heteroaryl optionally substituted with 1-4 (e.g., 1-3. 1-2. 1) R⁴²; wherein each —(C₁₋₄ alkylene) is, independently, optionally substituted with one or more substituents as defined herein.

In some embodiments of Formula IV, R³⁶ is selected from the group consisting of —(CH₂)heterocyclyl optionally substituted with 1-2 (e.g., 1) R⁴¹ and —(CH₂)heteroaryl optionally substituted with 1-2 (e.g., 1) R⁴².

In some embodiments of Formula IV, each R³⁷ is independently selected from the group consisting of halide (e.g., F, Cl, Br, I), unsubstituted —(C₁₋₉ alkyl) (e.g., C₁₋₈, C₁₋₇, C₁₋₆, C₁₋₅, C₁₋₄, C₁₋₃, C₁₋₂, C₁), unsubstituted —(C₂₋₉ alkenyl) (e.g., C₂₋₈, C₂₋₇, C₂₋₆, C₂₋₅, C₂₋₄, C₂₋₃, C₂), unsubstituted —(C₂₋₉ alkynyl) (e.g., C₂₋₈, C₂₋₇, C₂₋₆, C₂₋₅, C₂₋₄, C₂₋₃, C₂), and unsubstituted —(C₁₋₉ haloalkyl) (e.g., C₁₋₈, C₁₋₇, C₁₋₆, C₁₋₅, C₁₋₄, C₁₋₃, C₁₋₂, C₁).

In some embodiments of Formula IV, R³⁷ is selected from the group consisting of F, Cl, Br, unsubstituted —(C₁₋₄ alkyl) (e.g., C₁₋₃, C₁₋₂, C₁), unsubstituted —(C₂₋₄ alkenyl) (e.g., C₂₋₃, C₂), unsubstituted —(C₂₋₄ alkynyl) (e.g., C₂₋₃, C₂), and unsubstituted —(C₁₋₄ haloalkyl) (e.g., C₁₋₃, C₁₋₂, C₁).

In some embodiments of Formula IV, R³⁷ is selected from the group consisting of H, F, Cl, unsubstituted —(C₁₋₂ alkyl) (e.g., Me), unsubstituted —(C₂ alkenyl), unsubstituted —(C₂ alkynyl), and unsubstituted —(C₁₋₂ haloalkyl) (e.g., C₁).

In some embodiments of Formula IV, R³⁷ is selected from the group consisting of H, F, Cl, Me, and —CF₃.

In some embodiments of Formula IV, R³⁸ is -heterocyclyl optionally substituted with 1-10 (e.g., 1-9, 1-8, 1-7, 1-6, 1-5, 1-4, 1-3. 1-2. 1) R⁴⁴.

In some embodiments of Formula IV, R³⁹ is selected from the group consisting of —(C₁₋₄ alkylene)heterocyclyl optionally substituted with 1-10 (e.g., 1-9, 1-8, 1-7, 1-6, 1-5, 1-4, 1-3, 1-2, 1) R⁴⁵ and —(C₁₋₄ alkylene)heteroaryl optionally substituted with 1-10 (e.g., 1-9, 1-8, 1-7, 1-6, 1-5, 1-4, 1-3, 1-2, 1) R⁴⁶; wherein each —(C₁₋₄ alkylene) is, independently, optionally substituted with one or more substituents as defined herein.

In some embodiments of Formula IV, R³⁹ is selected from the group consisting of —(C₁₋₂ alkylene)heterocyclyl optionally substituted with 1-4 (e.g., 1-3. 1-2. 1) R⁴⁵ and —(C₁₋₂ alkylene)heteroaryl optionally substituted with 1-4 (e.g., 1-3. 1-2. 1) R⁴⁶; wherein each —(C₁₋₄ alkylene) is, independently, optionally substituted with one or more substituents as defined herein.

In some embodiments of Formula IV, R³⁹ is selected from the group consisting of —(CH₂)heterocyclyl optionally substituted with 1-2 (e.g., 1) R⁴⁵ and —(CH₂)heteroaryl optionally substituted with 1-2 (e.g., 1) R⁴⁶.

In some embodiments of Formula IV, each R⁴⁰ is independently selected from the group consisting of halide (e.g., F, Cl, Br, I), unsubstituted —(C₁₋₉ alkyl) (e.g., C₁₋₈, C₁₋₇, C₁₋₆, C₁₋₅, C₁₋₄, C₁₋₃, C₁₋₂, C₁), unsubstituted —(C₂₋₉ alkenyl) (e.g., C₂₋₈, C₂₋₇, C₂₋₆, C₂₋₅, C₂₋₄, C₂₋₃, C₂), unsubstituted —(C₂₋₉ alkynyl) (e.g., C₂₋₈, C₂₋₇, C₂₋₆, C₂₋₅, C₂₋₄, C₂₋₃, C₂), unsubstituted —(C₁₋₉ haloalkyl) (e.g., C₁₋₈, C₁₋₇, C₁₋₆, C₁₋₅, C₁₋₄, C₁₋₃, C₁₋₂, C₁), and —(C₁₋₄ alkylene)ₚOR⁴³; wherein —(C₁₋₄ alkylene) is optionally substituted with one or more substituents as defined herein.

In some embodiments of Formula IV, R⁴⁰ is selected from the group consisting of F, Cl, Br, unsubstituted —(C₁₋₄ alkyl) (e.g., C₁₋₃, C₁₋₂, C₁), unsubstituted —(C₂₋₄ alkenyl) (e.g., C₂₋₃, C₂), unsubstituted —(C₂₋₄ alkynyl) (e.g., C₂₋₃, C₂), unsubstituted —(C₁₋₄ haloalkyl) (e.g., C₁₋₃, C₁₋₂, C₁), —(C₁₋₂ alkylene)ₚOH, and —(C₁₋₂ alkylene)ₚO(C₁₋₄ alkyl) (e.g., C₁₋₃, C₁₋₂, C₁); wherein each —(C₁₋₄ alkylene) is, independently, optionally substituted with one or more substituents as defined herein.

In some embodiments of Formula IV, R⁴⁰ is selected from the group consisting of F, Cl, unsubstituted —(C₁₋₂ alkyl) (e.g., Me), unsubstituted —(C₂ alkenyl), unsubstituted —(C₂ alkynyl), unsubstituted —(C₁₋₂ haloalkyl) (e.g., C₁), —(CH₂)ₚOH, and —(CH₂)ₚO(C₁₋₂ alkyl) (e.g., Me).

In some embodiments of Formula IV, R⁴⁰ is selected from the group consisting of F, Cl, Me, —CF₃, —OH, and —OMe.

In some embodiments of Formula IV, each R⁴¹ is independently selected from the group consisting of halide (e.g., F, Cl, Br, I), unsubstituted —(C₁₋₉ alkyl) (e.g., C₁₋₈, C₁₋₇, C₁₋₆, C₁₋₅, C₁₋₄, C₁₋₃, C₁₋₂, C₁), and

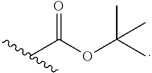 [SMILES: *C(=O)OC(C)(C)C]

In some embodiments of Formula IV, each R⁴¹ is independently selected from the group consisting of F, Cl, Br, and unsubstituted —(C₁₋₄ alkyl) (e.g., C₁₋₃, C₁₋₂, C₁).

In some embodiments of Formula IV, each R⁴¹ is independently selected from the group consisting of F, Cl, Br, and Me.

In some embodiments of Formula IV, each R⁴² is independently selected from the group consisting of halide (e.g., F, Cl, Br, I) and unsubstituted —(C₁₋₉ alkyl) (e.g., C₁₋₈, C₁₋₇, C₁₋₆, C₁₋₅, C₁₋₄, C₁₋₃, C₁₋₂, C₁).

In some embodiments of Formula IV, each R⁴² is independently selected from the group consisting of F, Cl, Br, and unsubstituted —(C₁₋₄ alkyl) (e.g., C₁₋₃, C₁₋₂, C₁).

In some embodiments of Formula IV, each R⁴² is independently selected from the group consisting of F, Cl, Br, and Me.

In some embodiments of Formula IV, each R⁴³ is independently selected from the group consisting of H and unsubstituted —(C₁₋₉ alkyl) (e.g., C₁₋₈, C₁₋₇, C₁₋₆, C₁₋₅, C₁₋₄, C₁₋₃, C₁₋₂, C₁).

In some embodiments of Formula IV, each R⁴³ is independently selected from the group consisting of H and unsubstituted —(C₁₋₄ alkyl) (e.g., C₁₋₃, C₁₋₂, C₁).

In some embodiments of Formula IV, each R⁴³ is independently selected from the group consisting of H and Me.

In some embodiments of Formula IV, each R⁴⁴ is independently selected from the group consisting of halide (e.g., F, Cl, Br, I), unsubstituted —(C₁₋₉ alkyl) (e.g., C₁₋₈, C₁₋₇, C₁₋₆, C₁₋₅, C₁₋₄, C₁₋₃, C₁₋₂, C₁), unsubstituted —(C₂₋₉ alkenyl) (e.g., C₂₋₈, C₂₋₇, C₂₋₆, C₂₋₅, C₂₋₄, C₂₋₃, C₂), unsubstituted —(C₂₋₉ alkynyl) (e.g., C₂₋₈, C₂₋₇, C₂₋₆, C₂₋₅, C₂₋₄, C₂₋₃, C₂), unsubstituted —(C₁₋₉ haloalkyl) (e.g., C₁₋₈, C₁₋₇, C₁₋₆, C₁₋₅, C₁₋₄, C₁₋₃, C₁₋₂, C₁), and —(C₁₋₄ alkylene)ₚOR⁴⁷; wherein —(C₁₋₄ alkylene) is optionally substituted with one or more substituents as defined herein.

In some embodiments of Formula IV, R⁴⁴ is selected from the group consisting of F, Cl, Br, unsubstituted —(C₁₋₄ alkyl) (e.g., C₁₋₃, C₁₋₂, C₁), unsubstituted —(C₂₋₄ alkenyl) (e.g., C₂₋₃, C₂), unsubstituted —(C₂₋₄ alkynyl) (e.g., C₂₋₃, C₂), unsubstituted —(C₁₋₄ haloalkyl) (e.g., C₁₋₃, C₁₋₂, C₁), —(C₁₋₂ alkylene)ₚOH, and —(C₁₋₂ alkylene)ₚO(C₁₋₄ alkyl) (e.g., C₁₋₃, C₁₋₂, C₁); wherein each —(C₁₋₄ alkylene) is, independently, optionally substituted with one or more substituents as defined herein.

In some embodiments of Formula IV, R⁴⁴ is selected from the group consisting of F, Cl, unsubstituted —(C₁₋₂ alkyl) (e.g., Me), unsubstituted —(C₂ alkenyl), unsubstituted —(C₂ alkynyl), unsubstituted —(C₁₋₂ haloalkyl) (e.g., C₁), —(CH₂)ₚOH, and —(CH₂)ₚO(C₁₋₂ alkyl) (e.g., Me).

In some embodiments of Formula IV, R⁴⁴ is selected from the group consisting of F, Cl, Me, —CF₃, —OH, and —OMe.

In some embodiments of Formula IV, each R⁴⁵ is independently selected from the group consisting of halide (e.g., F, Cl, Br, I), unsubstituted —(C₁₋₉ alkyl) (e.g., C₁₋₈, C₁₋₇, C₁₋₆, C₁₋₅, C₁₋₄, C₁₋₃, C₁₋₂, C₁), and

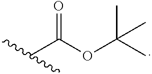 [SMILES: *C(=O)OC(C)(C)C]

In some embodiments of Formula IV, each R⁴⁵ is independently selected from the group consisting of F, Cl, Br, and unsubstituted —(C₁₋₄ alkyl) (e.g., C₁₋₃, C₁₋₂, C₁).

In some embodiments of Formula IV, each R⁴⁵ is independently selected from the group consisting of F, Cl, Br, and Me.

In some embodiments of Formula IV, each R⁴⁶ is independently selected from the group consisting of halide (e.g., F, Cl, Br, I) and unsubstituted —(C₁₋₉ alkyl) (e.g., C₁₋₈, C₁₋₇, C₁₋₆, C₁₋₅, C₁₋₄, C₁₋₃, C₁₋₂, C₁).

In some embodiments of Formula IV, each R⁴⁶ is independently selected from the group consisting of F, Cl, Br, and unsubstituted —(C₁₋₄ alkyl) (e.g., C₁₋₃, C₁₋₂, C₁).

In some embodiments of Formula IV, each R⁴⁶ is independently selected from the group consisting of F, Cl, Br, and Me.

In some embodiments of Formula IV, each R⁴⁷ is independently selected from the group consisting of H and unsubstituted —(C₁₋₉ alkyl) (e.g., C₁₋₈, C₁₋₇, C₁₋₆, C₁₋₅, C₁₋₄, C₁₋₃, C₁₋₂, C₁).

In some embodiments of Formula IV, each R⁴⁷ is independently selected from the group consisting of H and unsubstituted —(C₁₋₄ alkyl) (e.g., C₁₋₃, C₁₋₂, C₁).

In some embodiments of Formula IV, each R⁴⁷ is independently selected from the group consisting of H and Me.

In some embodiments, provided herein is a compound, or a pharmaceutically acceptable salt thereof, of Formula IV:

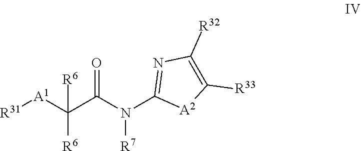

wherein:
R³, R⁴, and R⁵ are independently selected from the group consisting of H, halide, unsubstituted —(C₁₋₆ alkyl), unsubstituted —(C₁₋₄ haloalkyl), —OR¹⁰, and —CN;
each R⁶ is independently selected from the group consisting of H, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
R⁷ is selected from the group consisting of H, unsubstituted —(C₁₋₉ alkyl), unsubstituted —(C₃₋₉ alkenyl), unsubstituted —(C₃₋₉ alkynyl), unsubstituted —(C₁₋₉ haloalkyl), —(C₁₋₄ alkylene)C(═O)OR¹¹, —(C₁₋₄ alkylene)C(═O)N(R¹²)₂, —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein;
each R¹⁰ is independently selected from the group consisting of H, unsubstituted —(C₁₋₉ alkyl), unsubstituted —(C₃₋₉ alkenyl), unsubstituted —(C₃₋₉ alkynyl), and unsubstituted —(C₁₋₉ haloalkyl);
R¹¹ is selected from the group consisting of H and unsubstituted —(C₁₋₉ alkyl);
each R¹² is independently selected from the group consisting of H and substituted or unsubstituted —(C₁₋₉ alkyl);
each R¹³ is independently selected from the group consisting of halide, unsubstituted —(C₁₋₉ alkyl), unsubstituted —(C₁₋₉ haloalkyl), and —OR¹⁰;
each R¹⁴ is independently selected from the group consisting of halide, unsubstituted —(C₁₋₉ alkyl), unsubstituted —(C₁₋₉ haloalkyl), and —OR¹⁰;
each R¹⁵ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁶ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁷ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R³⁰ is selected from the group consisting of H and unsubstituted —(C₁₋₉ alkyl);
R³¹ is selected from the group consisting of

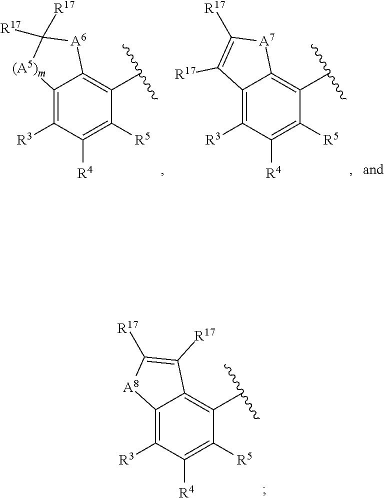

R³² is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -carbocyclyl optionally substituted with 1-10 R³⁴, —C(═O)R³⁵, C(═O)NHR³⁶, and —CN;
R³³ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -carbocyclyl optionally substituted with 1-10 R³⁷, —C(═O)R³⁸, —C(═O)NHR³⁹, and —CN;
each R³⁴ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
R³⁵ is -heterocyclyl optionally substituted with 1-10 R⁴⁰;
R³⁶ is selected from the group consisting of —(C₁₋₄ alkylene)heterocyclyl optionally substituted with 1-10 R⁴¹ and —(C₁₋₄ alkylene)heteroaryl optionally substituted with 1-10 R⁴²; wherein each —(C₁₋₄ alkylene) is, independently, optionally substituted with one or more substituents as defined herein;
each R³⁷ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
R³⁸ is -heterocyclyl optionally substituted with 1-10 R⁴⁴;
R³⁹ is selected from the group consisting of —(C₁₋₄ alkylene)heterocyclyl optionally substituted with 1-10 R⁴⁵ and —(C₁₋₄ alkylene)heteroaryl optionally substituted with 1-10 R⁴⁶; wherein each —(C₁₋₄ alkylene) is, independently, optionally substituted with one or more substituents as defined herein;
each R⁴⁰ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(C₁₋₄ alkylene)ₚOR⁴³; wherein —(C₁₋₄ alkylene) is optionally substituted with one or more substituents as defined herein;
each R⁴¹ is independently selected from the group consisting of halide, unsubstituted —(C₁₋₉ alkyl), and

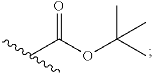 [SMILES: *C(=O)OC(C)(C)C]

each R⁴² is independently selected from the group consisting of halide and unsubstituted —(C₁₋₉ alkyl);
each R⁴³ is independently selected from the group consisting of H and unsubstituted —(C₁₋₉ alkyl);
each R⁴⁴ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(C₁₋₄ alkylene)ₚOR⁴⁷; wherein —(C₁₋₄ alkylene) is optionally substituted with one or more substituents as defined herein;
each R⁴⁵ is independently selected from the group consisting of halide, unsubstituted —(C₁₋₉ alkyl), and

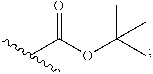 [SMILES: *C(=O)OC(C)(C)C]

each R⁴⁶ is independently selected from the group consisting of halide and unsubstituted —(C₁₋₉ alkyl);
each R⁴⁷ is independently selected from the group consisting of H and unsubstituted —(C₁₋₉ alkyl);
A¹ is selected from the group consisting of —C(R¹⁵)₂—, O, and S;
A² is selected from the group consisting of O and S;
each A⁵ is independently selected from the group consisting of —C(R¹⁶)₂—, —N(R¹⁰)—, O, and S;
with the proviso that two A⁵ are not both N, O or S;
A⁶ is selected from the group consisting of —C(R¹⁶)₂—, —N(R¹⁰)—, O, and S;
with the proviso that A⁵ and A⁶ cannot both be C(R¹⁶)₂;
A⁷ is selected from the group consisting of —N(R³⁰)—, O and S;
A⁸ is selected from the group consisting of —N(R³⁰)—, O and S;

m is 1 or 2; and

each p independently is 0 or 1.

For example, provided herein is a compound, or a pharmaceutically acceptable salt thereof, of Formula IV:

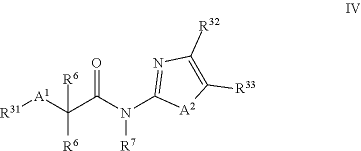

wherein:
R³, R⁴, and R⁵ are independently selected from the group consisting of H, halide, unsubstituted —(C₁₋₆ alkyl), unsubstituted —(C₁₋₄ haloalkyl), —ORI⁰, and —CN;
each R⁶ is independently selected from the group consisting of H, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
R⁷ is selected from the group consisting of H, unsubstituted —(C₁₋₉ alkyl), unsubstituted —(C₃₋₉ alkenyl), unsubstituted —(C₃₋₉ alkynyl), unsubstituted —(C₁₋₉ haloalkyl), —(C₁₋₄ alkylene)C(═O)OR¹¹, —(C₁₋₄ alkylene)C(═O)N(R¹²)₂, —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein;
each R¹⁰ is independently selected from the group consisting of H, unsubstituted —(C₁₋₉ alkyl), unsubstituted —(C₃₋₉ alkenyl), unsubstituted —(C₃₋₉ alkynyl), and unsubstituted —(C₁₋₉ haloalkyl);
R¹¹ is selected from the group consisting of H and unsubstituted —(C₁₋₉ alkyl);
each R¹² is independently selected from the group consisting of H and substituted or unsubstituted —(C₁₋₉ alkyl);
each R¹³ is independently selected from the group consisting of halide, unsubstituted —(C₁₋₉ alkyl), unsubstituted —(C₁₋₉ haloalkyl), and —OR¹⁰;
each R¹⁴ is independently selected from the group consisting of halide, unsubstituted —(C₁₋₉ alkyl), unsubstituted —(C₁₋₉ haloalkyl), and —OR¹⁰;
each R¹⁵ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁶ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁷ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R³⁰ is selected from the group consisting of H and unsubstituted —(C₁₋₉ alkyl);
R³¹ is selected from the group consisting of

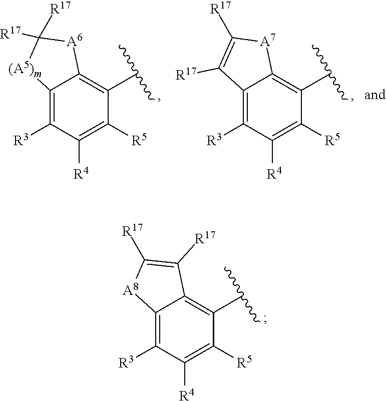

R³² is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -carbocyclyl optionally substituted with 1-10 R³⁴, —C(═O)R³⁵, C(═O)NHR³⁶, and —CN;
R³³ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -carbocyclyl optionally substituted with 1-10 R³⁷, —C(═O)R³⁸, —C(═O)NHR³⁹, and —CN;
each R³⁴ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
R³⁵ is -heterocyclyl optionally substituted with 1-10 R⁴⁰;
R³⁶ is selected from the group consisting of —(C₁₋₄ alkylene)heterocyclyl optionally substituted with 1-10 R⁴¹ and —(C₁₋₄ alkylene)heteroaryl optionally substituted with 1-10 R⁴²; wherein each —(C₁₋₄ alkylene) is, independently, optionally substituted with one or more substituents as defined herein;
each R³⁷ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
R³⁸ is -heterocyclyl optionally substituted with 1-10 R⁴⁴;
R³⁹ is selected from the group consisting of —(C₁₋₄ alkylene)heterocyclyl optionally substituted with 1-10 R⁴⁵ and —(C₁₋₄ alkylene)heteroaryl optionally substituted with 1-10 R⁴⁶; wherein each —(C₁₋₄ alkylene) is, independently, optionally substituted with one or more substituents as defined herein;
each R⁴⁰ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(C₁₋₄ alkylene)ₚOR⁴³; wherein —(C₁₋₄ alkylene) is optionally substituted with one or more substituents as defined herein;
each R⁴¹ is independently selected from the group consisting of halide, unsubstituted —(C₁₋₉ alkyl), and

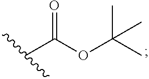 [SMILES: *C(=O)OC(C)(C)C]

each R⁴² is independently selected from the group consisting of halide and unsubstituted —(C₁₋₉ alkyl);
each R⁴³ is independently selected from the group consisting of H and unsubstituted —(C₁₋₉ alkyl);
each R⁴⁴ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(C₁₋₄ alkylene)ₚOR⁴⁷; wherein —(C₁₋₄ alkylene) is optionally substituted with one or more substituents as defined herein;
each R⁴⁵ is independently selected from the group consisting of halide, unsubstituted —(C₁₋₉ alkyl), and

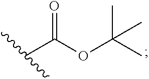 [SMILES: *C(=O)OC(C)(C)C]

each R⁴⁶ is independently selected from the group consisting of halide and unsubstituted —(C₁₋₉ alkyl);
each R⁴⁷ is independently selected from the group consisting of H and unsubstituted —(C₁₋₉ alkyl);
A¹ is selected from the group consisting of —C(R¹⁵)₂—, O, and S;
A² is selected from the group consisting of O and S;
each A⁵ is independently selected from the group consisting of —C(R¹⁶)₂—, —N(R¹⁰)—, O, and S;
with the proviso that two A⁵ are not both N, O or S;
A⁶ is selected from the group consisting of —C(R¹⁶)₂—, —N(R¹⁰)—, O, and S;
with the proviso that A⁵ and A⁶ cannot both be C(R¹⁶)₂;
A⁷ is selected from the group consisting of —N(R³⁰)—, O and S;
A⁸ is selected from the group consisting of —N(R³⁰)—, O and S;

m is 1 or 2;

each p independently is 0 or 1; and
with the proviso that Formula IV is not a structure selected from the group consisting of:

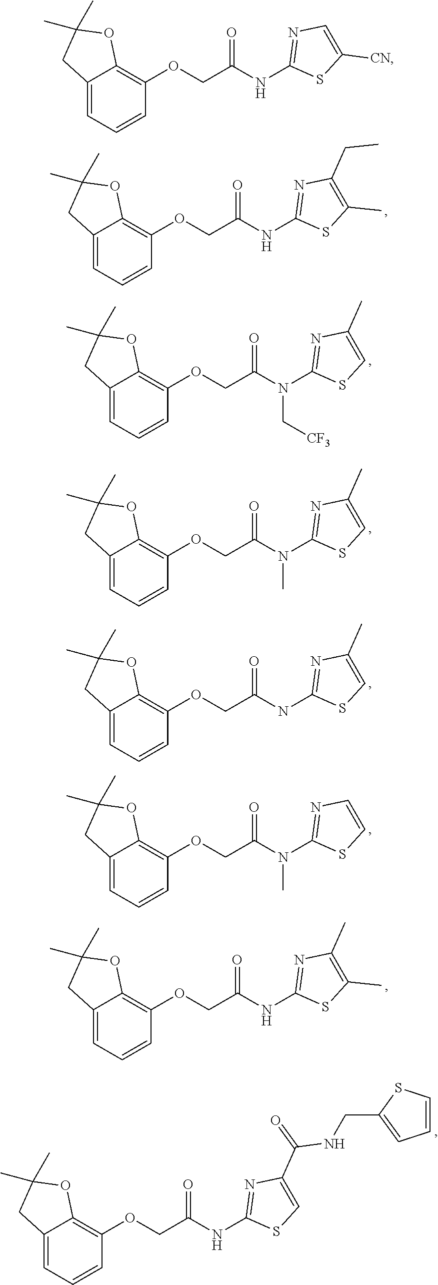

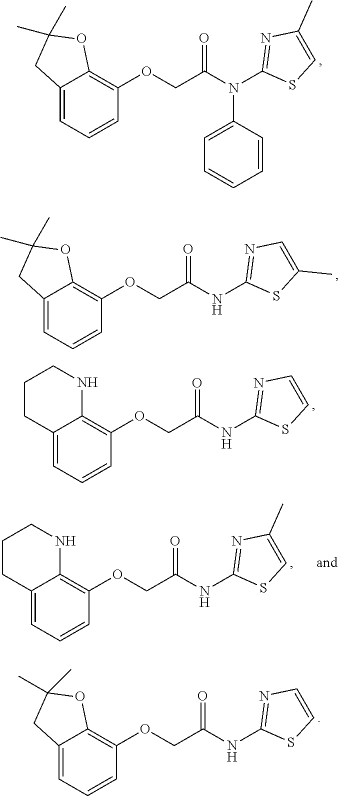

In some embodiments, provided herein is a compound, or a pharmaceutically acceptable salt thereof, of Formula IV:

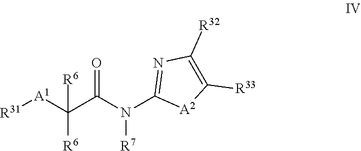

wherein:
R³, R⁴, and R⁵ are independently selected from the group consisting of H, halide, unsubstituted —(C₁₋₆ alkyl), unsubstituted —(C₁₋₄ haloalkyl), —OR¹⁰, and —CN;
each R⁶ is independently selected from the group consisting of H, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
R⁷ is selected from the group consisting of H, unsubstituted —(C₁₋₉ alkyl), unsubstituted —(C₃₋₉ alkenyl), unsubstituted —(C₃₋₉ alkynyl), unsubstituted —(C₁₋₉ haloalkyl), —(C₁₋₄ alkylene)C(═O)OR¹¹, —(C₁₋₄ alkylene)C(═O)N(R¹²)₂, —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein;
each R¹⁰ is independently selected from the group consisting of H, unsubstituted —(C₁₋₉ alkyl), unsubstituted —(C₃₋₉ alkenyl), unsubstituted —(C₃₋₉ alkynyl), and unsubstituted —(C₁₋₉ haloalkyl);
R¹¹ is selected from the group consisting of H and unsubstituted —(C₁₋₉ alkyl);
each R¹² is independently selected from the group consisting of H and substituted or unsubstituted —(C₁₋₉ alkyl);
each R¹³ is independently selected from the group consisting of halide, unsubstituted —(C₁₋₉ alkyl), unsubstituted —(C₁₋₉ haloalkyl), and —OR¹⁰;
each R¹⁴ is independently selected from the group consisting of halide, unsubstituted —(C₁₋₉ alkyl), unsubstituted —(C₁₋₉ haloalkyl), and —OR¹⁰;
each R¹⁵ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁶ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁷ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R³⁰ is selected from the group consisting of H and unsubstituted —(C₁₋₉ alkyl);
R³¹ is selected from the group consisting of

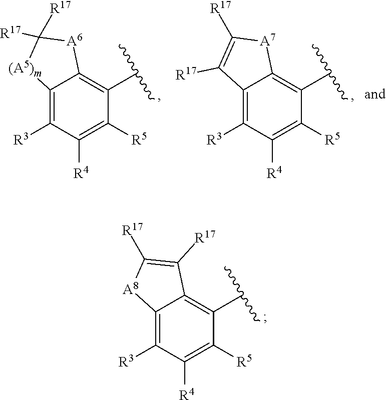

R³² is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -carbocyclyl optionally substituted with 1-10 R³⁴, —C(═O)R³⁵, —C(═O)NHR³⁶, and —CN;
R³³ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -carbocyclyl optionally substituted with 1-10 R³⁷, —C(═O)R³⁸, —C(═O)NHR³⁹, and —CN;
each R³⁴ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
R³⁵ is -heterocyclyl optionally substituted with 1-10 R⁴⁰;
R³⁶ is selected from the group consisting of —(C₁₋₄ alkylene)heterocyclyl optionally substituted with 1-10 R⁴¹ and —(C₁₋₄ alkylene)heteroaryl optionally substituted with 1-10 R⁴²; wherein each —(C₁₋₄ alkylene) is, independently, optionally substituted with one or more substituents as defined herein;
each R³⁷ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
R³⁸ is -heterocyclyl optionally substituted with 1-10 R⁴⁴;
R³⁹ is selected from the group consisting of —(C₁₋₄ alkylene)heterocyclyl optionally substituted with 1-10 R⁴⁵ and —(C₁₋₄ alkylene)heteroaryl optionally substituted with 1-10 R⁴⁶; wherein each —(C₁₋₄ alkylene) is, independently, optionally substituted with one or more substituents as defined herein;
each R⁴⁰ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(C₁₋₄ alkylene)ₚOR⁴³; wherein —(C₁₋₄ alkylene) is optionally substituted with one or more substituents as defined herein;
each R⁴¹ is independently selected from the group consisting of halide, unsubstituted —(C₁₋₉ alkyl), and

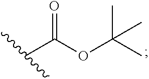 [SMILES: *C(=O)OC(C)(C)C]

each R⁴² is independently selected from the group consisting of halide and unsubstituted —(C₁₋₉ alkyl);
each R⁴³ is independently selected from the group consisting of H and unsubstituted —(C₁₋₉ alkyl);
each R⁴⁴ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(C₁₋₄ alkylene)ₚOR⁴⁷; wherein —(C₁₋₄ alkylene) is optionally substituted with one or more substituents as defined herein;
each R⁴⁵ is independently selected from the group consisting of halide, unsubstituted —(C₁₋₉ alkyl), and

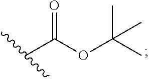 [SMILES: *C(=O)OC(C)(C)C]

each R⁴⁶ is independently selected from the group consisting of halide and unsubstituted —(C₁₋₉ alkyl);
each R⁴⁷ is independently selected from the group consisting of H and unsubstituted —(C₁₋₉ alkyl);
A¹ is selected from the group consisting of —C(R¹⁵)₂—, O, and S;
A² is selected from the group consisting of O and S;
each A⁵ is independently selected from the group consisting of —C(R¹⁶)₂—, —N(R¹⁰)—, O, and S;
with the proviso that two A⁵ are not both N, O or S;
A⁶ is selected from the group consisting of —C(R¹⁶)₂—, —N(R¹⁰)—, O, and S;
with the proviso that A⁵ and A⁶ cannot both be C(R¹⁶)₂;
A⁷ is selected from the group consisting of —N(R³⁰)—, O and S;
A⁸ is selected from the group consisting of —N(R³⁰)—, O and S;
m is 1 or 2; and
each p is 0 or 1.

For example, provided herein is a compound, or a pharmaceutically acceptable salt thereof, of Formula IV:

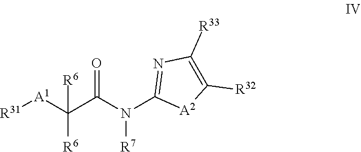

wherein:
R³, R⁴, and R⁵ are independently selected from the group consisting of H, halide, unsubstituted —(C₁₋₆ alkyl), unsubstituted —(C₁₋₄ haloalkyl), —OR¹⁰, and —CN;
each R⁶ is independently selected from the group consisting of H, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
R⁷ is selected from the group consisting of H, unsubstituted —(C₁₋₉ alkyl), unsubstituted —(C₃₋₉ alkenyl), unsubstituted —(C₃₋₉ alkynyl), unsubstituted —(C₁₋₉ haloalkyl), —(C₁₋₄ alkylene)C(═O)OR¹¹, —(C₁₋₄ alkylene)C(═O)N(R¹²)₂, —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein;
each R¹⁰ is independently selected from the group consisting of H, unsubstituted —(C₁₋₉ alkyl), unsubstituted —(C₃₋₉ alkenyl), unsubstituted —(C₃₋₉ alkynyl), and unsubstituted —(C₁₋₉ haloalkyl);
R¹¹ is selected from the group consisting of H and unsubstituted —(C₁₋₉ alkyl);
each R¹² is independently selected from the group consisting of H and substituted or unsubstituted —(C₁₋₉ alkyl);
each R¹³ is independently selected from the group consisting of halide, unsubstituted —(C₁₋₉ alkyl), unsubstituted —(C₁₋₉ haloalkyl), and —OR¹⁰;
each R¹⁴ is independently selected from the group consisting of halide, unsubstituted —(C₁₋₉ alkyl), unsubstituted —(C₁₋₉ haloalkyl), and —OR¹⁰;
each R¹⁵ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁶ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁷ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R³⁰ is selected from the group consisting of H and unsubstituted —(C₁₋₉ alkyl);
R³¹ is selected from the group consisting of

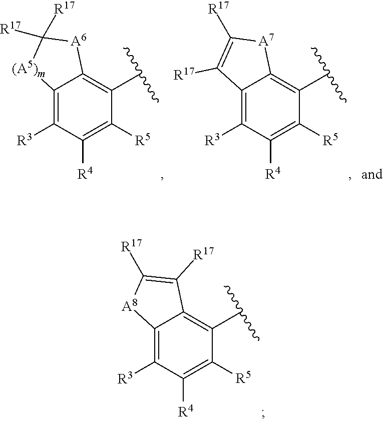

with the proviso that when R³¹ is

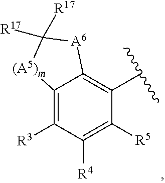

A¹ is O, m is 1 or 2, A⁵ is CH₂, both R⁶ are H and R⁷ is H, then R³² and R³³ are not selected from the group consisting of H, halide, unsubstituted —(C₁₋₃ alkyl), —CN, and

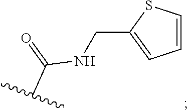 [SMILES: *C(=O)NCc1cccs1]

with the proviso that when R³¹ is

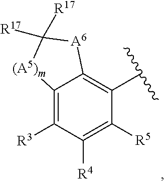

A¹ is O, m is 1, A⁵ is CH₂, both R⁶ are H and R⁷ is selected from the group consisting of unsubstituted —(C₁₋₂ alkyl), unsubstituted —(C₁₋₂ haloalkyl), -aryl optionally substituted with 1-5 R¹⁴, and R¹⁴ is halide or methyl, then R³² and R³³ are not selected from the group consisting of H, halide, and unsubstituted —(C₁₋₂ alkyl);
R³² is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -carbocyclyl optionally substituted with 1-10 R³⁴, —C(═O)R³⁵, C(═O)NHR³⁶, and —CN;
R³³ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -carbocyclyl optionally substituted with 1-10 R³⁷, —C(═O)R³⁸, —C(═O)NHR³⁹, and —CN;
each R³⁴ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
R³⁵ is -heterocyclyl optionally substituted with 1-10 R⁴⁰;
R³⁶ is selected from the group consisting of —(C₁₋₄ alkylene)heterocyclyl optionally substituted with 1-10 R⁴¹ and —(C₁₋₄ alkylene)heteroaryl optionally substituted with 1-10 R⁴²; wherein each —(C₁₋₄ alkylene) is, independently, optionally substituted with one or more substituents as defined herein;
each R³⁷ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
R³⁸ is -heterocyclyl optionally substituted with 1-10 R⁴⁴;
R³⁹ is selected from the group consisting of —(C₁₋₄ alkylene)heterocyclyl optionally substituted with 1-10 R⁴⁵ and —(C₁₋₄ alkylene)heteroaryl optionally substituted with 1-10 R⁴⁶; wherein each —(C₁₋₄ alkylene) is, independently, optionally substituted with one or more substituents as defined herein;
each R⁴⁰ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(C₁₋₄ alkylene)ₚOR⁴³; wherein —(C₁₋₄ alkylene) is optionally substituted with one or more substituents as defined herein;
each R⁴¹ is independently selected from the group consisting of halide, unsubstituted —(C₁₋₉ alkyl), and

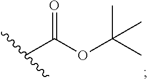 [SMILES: *C(=O)OC(C)(C)C]

each R⁴² is independently selected from the group consisting of halide and unsubstituted —(C₁₋₉ alkyl);
each R⁴³ is independently selected from the group consisting of H and unsubstituted —(C₁₋₉ alkyl);
each R⁴⁴ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(C₁₋₄ alkylene)ₚOR⁴⁷; wherein —(C₁₋₄ alkylene) is optionally substituted with one or more substituents as defined herein;
each R⁴⁵ is independently selected from the group consisting of halide, unsubstituted —(C₁₋₉ alkyl), and

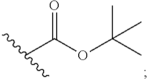 [SMILES: *C(=O)OC(C)(C)C]

each R⁴⁶ is independently selected from the group consisting of halide and unsubstituted —(C₁₋₉ alkyl);
each R⁴⁷ is independently selected from the group consisting of H and unsubstituted —(C₁₋₉ alkyl);
A¹ is selected from the group consisting of —C(R¹⁵)₂—, O, and S;
A² is selected from the group consisting of O and S;
each A⁵ is independently selected from the group consisting of —C(R¹⁶)₂—, —N(R¹⁰)—, O, and S;
with the proviso that two A⁵ are not both N, O or S;
A⁶ is selected from the group consisting of —C(R¹⁶)₂—, —N(R¹⁰)—, O, and S;
with the proviso that A⁵ and A⁶ cannot both be C(R¹⁶)₂;
A⁷ is selected from the group consisting of —N(R³⁰)—, O and S;
A⁸ is selected from the group consisting of —N(R³⁰)—, O and S;
m is 1 or 2; and
each p is 0 or 1.

Some embodiments of the present disclosure include compounds of Formula V:

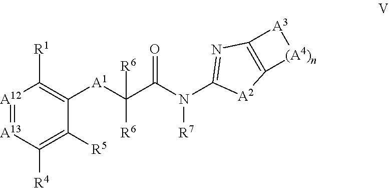

or enantiomers, diastereomers, tautomers, prodrugs, and pharmaceutically acceptable salts thereof.

In some embodiments of Formula V,

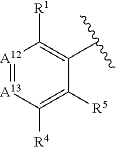

is independently selected from the group consisting of

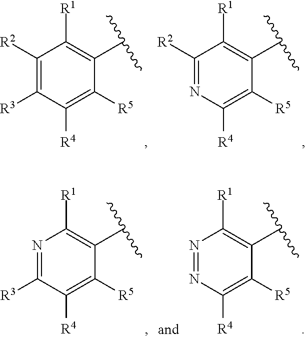

In some embodiments of Formula V, R¹, R², R³, R⁴, and R⁵ are independently selected from the group consisting of H, halide (e.g., F, Cl, Br, I), unsubstituted —(C₁₋₉ alkyl) (e.g., C₁₋₈, C₁₋₇, C₁₋₆, C₁₋₅, C₁₋₄, C₁₋₃, C₁₋₂, C₁), unsubstituted —(C₂₋₉ alkenyl) (e.g., C₂₋₈, C₂₋₇, C₂₋₆, C₂₋₅, C₂₋₄, C₂₋₃, C₂), unsubstituted —(C₂₋₉ alkynyl) (e.g., C₂₋₈, C₂₋₇, C₂₋₆, C₂₋₅, C₂₋₄, C₂₋₃, C₂), unsubstituted —(C₁₋₉ haloalkyl) (e.g., C₁₋₈, C₁₋₇, C₁₋₆, C₁₋₅, C₁₋₄, C₁₋₃, C₁₋₂, C₁), —OR⁴⁸, -carbocyclyl optionally substituted with 1-4 (e.g., 1-3. 1-2. 1) R⁴⁹, and —CN;

In some embodiments of Formula V, R¹, R², R³, R⁴, and R⁵ are independently selected from the group consisting of H, F, Cl, Br, unsubstituted —(C₁₋₄ alkyl) (e.g., C₁₋₃, C₁₋₂, C₁), unsubstituted —(C₂₋₄ alkenyl) (e.g., C₂₋₃, C₂), unsubstituted —(C₂₋₄ alkynyl) (e.g., C₂₋₃, C₂), unsubstituted —(C₁₋₄ haloalkyl) (e.g., C₁₋₃, C₁₋₂, C₁), —O(C₁₋₄ alkyl) (e.g., C₁₋₃, C₁₋₂, C₁), -carbocyclyl optionally substituted with 1-2 (e.g., 1) R⁴⁹, and —CN.

In some embodiments of Formula V, R¹, R², R³, R⁴, and R⁵ are independently selected from the group consisting of F, Cl, unsubstituted —(C₁₋₂ alkyl) (e.g., Me), unsubstituted —(C₂ alkenyl), unsubstituted —(C₂ alkynyl), unsubstituted —(C₁₋₂ haloalkyl) (e.g., C₁), —O(C₁₋₂ alkyl) (e.g., Me), -carbocyclyl optionally substituted with 1 R⁴⁹, and —CN.

In some embodiments of Formula V, R¹, R², R³, R⁴, and R⁵ are independently selected from the group consisting of F, Cl, Me, —CF₃, —OMe, unsubstituted —(C₃-4)carbocyclyl, and —CN.

In some embodiments of Formula V, each R⁴⁸ is independently selected from the group consisting of unsubstituted —(C₁₋₉ alkyl) (e.g., C₁₋₈, C₁₋₇, C₁₋₆, C₁₋₅, C₁₋₄, C₁₋₃, C₁₋₂, C₁), unsubstituted —(C₃₋₉ alkenyl) (e.g., C₃₋₈, C₃₋₇, C₃₋₆, C₃₋₅, C₃₋₄, C₃), unsubstituted —(C₃₋₉ alkynyl) (e.g., C₃₋₈, C₃₋₇, C₃₋₆, C₃₋₅, C₃₋₄, C₃), and unsubstituted —(C₁₋₉ haloalkyl) (e.g., C₁₋₈, C₁₋₇, C₁₋₆, C₁₋₅, C₁₋₄, C₁₋₃, C₁₋₂, C₁).

In some embodiments of Formula V, each R⁴⁸ is independently selected from the group consisting of unsubstituted —(C₁₋₄ alkyl) (e.g., C₁₋₃, C₁₋₂, C₁), unsubstituted —(C₃₋₄ alkenyl) (e.g., C₃), unsubstituted —(C₃₋₄ alkynyl) (e.g., C₃), and unsubstituted —(C₁₋₄ haloalkyl) (e.g., C₁₋₃, C₁₋₂, C₁).

In some embodiments of Formula V, each R⁴⁸ is independently selected from the group consisting of unsubstituted —(C₁₋₂ alkyl) (e.g., Me) and unsubstituted —(C₁₋₂ haloalkyl) (e.g., C₁).

In some embodiments of Formula V, each R⁴⁸ is independently selected from the group consisting of Me and —CF₃.

In some embodiments of Formula V, each R⁴⁹ is independently selected from the group consisting of halide (e.g., F, Cl, Br, I), unsubstituted —(C₁₋₉ alkyl) (e.g., C₁₋₈, C₁₋₇, C₁₋₆, C₁₋₅, C₁₋₄, C₁₋₃, C₁₋₂, C₁), unsubstituted —(C₂₋₉ alkenyl) (e.g., C₂₋₈, C₂₋₇, C₂₋₆, C₂₋₅, C₂₋₄, C₂₋₃, C₂), unsubstituted —(C₂₋₉ alkynyl) (e.g., C₂₋₈, C₂₋₇, C₂₋₆, C₂₋₅, C₂₋₄, C₂₋₃, C₂), unsubstituted —(C₁₋₉ haloalkyl) (e.g., C₁₋₈, C₁₋₇, C₁₋₆, C₁₋₅, C₁₋₄, C₁₋₃, C₁₋₂, C₁), and —CN.

In some embodiments of Formula V, each R⁴⁹ is independently selected from the group consisting of F, Cl, Br, unsubstituted —(C₁₋₄ alkyl) (e.g., C₁₋₃, C₁₋₂, C₁), unsubstituted —(C₂₋₄ alkenyl) (e.g., C₂₋₃, C₂), unsubstituted —(C₂₋₄ alkynyl) (e.g., C₂₋₃, C₂), unsubstituted —(C₁₋₄ haloalkyl) (e.g., C₁₋₃, C₁₋₂, C₁), and —CN.

In some embodiments of Formula V, each R⁴⁹ is independently selected from the group consisting of F, Cl, unsubstituted —(C₁₋₂ alkyl) (e.g., Me), unsubstituted —(C₂ alkenyl), unsubstituted —(C₂ alkynyl), unsubstituted —(C₁₋₂ haloalkyl) (e.g., C₁), and —CN.

In some embodiments of Formula V, each R⁴⁹ is independently selected from the group consisting of F, Cl, Me, —CF₃, and —CN.

In some embodiments of Formula V, A¹² is selected from the group consisting of —C(R²)— and N.

In some embodiments of Formula V, A¹² is N.

In some embodiments of Formula V, A¹² is —C(R²)—.

In some embodiments of Formula V, A¹² is —CH—.

In some embodiments of Formula V, A¹³ is selected from the group consisting of —C(R³)— and N.

In some embodiments of Formula V, A¹³ is N.

In some embodiments of Formula V, A¹³ is —C(R²)—.

In some embodiments of Formula V, A¹³ is —CH—.

In some embodiments of Formula V, there is the proviso that when A¹² is —C(R²)— and A¹³ is —C(R³)—, then R¹, R², R³, R⁴, and R⁵ are not all H.

In some embodiments of Formula V, there is the proviso that when A¹² is N, then R¹, R³, R⁴, and R⁵ are not all H.

In some embodiments, provided herein is a compound, or a pharmaceutically acceptable salt thereof, of Formula V:

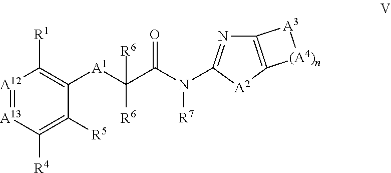

wherein:
R¹ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR⁴⁸, -carbocyclyl optionally substituted with 1-4 R⁴⁹, and —CN;
R² is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR⁴⁸, -carbocyclyl optionally substituted with 1-4 R⁴⁹, and —CN;
R³ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR⁴⁸, -carbocyclyl optionally substituted with 1-4 R⁴⁹, and —CN;
R⁴ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR⁴⁸, -carbocyclyl optionally substituted with 1-4 R⁴⁹, and —CN;
R⁵ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR⁴⁸, -carbocyclyl optionally substituted with 1-4 R⁴⁹, and —CN;
each R⁶ is independently selected from the group consisting of H, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
R⁷ is selected from the group consisting of unsubstituted —(C₁₋₉ alkyl), unsubstituted —(C₃₋₉ alkenyl), unsubstituted —(C₃₋₉ alkynyl), unsubstituted —(C₁₋₉ haloalkyl), —(C₁₋₄ alkylene)C(═O)OR¹¹, —(C₁₋₄ alkylene)C(═O)N(R¹²)₂, —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein;
each R¹⁰ is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
R¹¹ is selected from the group consisting of H and unsubstituted —(C₁₋₉ alkyl);
each R¹² is independently selected from the group consisting of H and substituted or unsubstituted —(C₁₋₉ alkyl);
each R¹³ is independently selected from the group consisting of halide, unsubstituted —(C₁₋₉ alkyl), unsubstituted —(C₁₋₉ haloalkyl), and —OR¹⁰;
each R¹⁴ is independently selected from the group consisting of unsubstituted —(C₁₋₉ alkyl), unsubstituted —(C₁₋₉ haloalkyl), and —OR¹⁰;
each R¹⁵ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁶ is independently selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
each R⁴⁸ is independently selected from the group consisting of unsubstituted —(C₁₋₉ alkyl), unsubstituted —(C₃₋₉ alkenyl), unsubstituted —(C₃₋₉ alkynyl), and unsubstituted —(C₁₋₉ haloalkyl);
each R⁴⁹ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
A¹ is selected from the group consisting of —C(R¹⁵)₂—, O, and S;
A² is selected from the group consisting of O and S;
A³ is selected from the group consisting of —C(R¹⁶)₂—, O and S;
each A⁴ is independently selected from the group consisting of —C(R¹⁶)₂—, O and S;
with the proviso that no more than two of A³ and/or A⁴ are O or S and that two O or S are never adjacent;
A¹² is selected from the group consisting of —C(R²)— and N;
A¹³ is selected from the group consisting of —C(R³)— and N;
n is 1 to 4; and
p is 0 to 1.

For example, provided herein is a compound, or a pharmaceutically acceptable salt thereof, of Formula V:

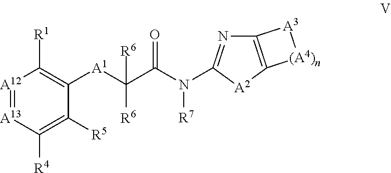

wherein:
R¹ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR⁴⁸, -carbocyclyl optionally substituted with 1-4 R⁴⁹, and —CN;
R² is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR⁴⁸, -carbocyclyl optionally substituted with 1-4 R⁴⁹, and —CN;
R³ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR⁴⁸, -carbocyclyl optionally substituted with 1-4 R⁴⁹, and —CN;
R⁴ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR⁴⁸, -carbocyclyl optionally substituted with 1-4 R⁴⁹, and —CN;
R⁵ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR⁴⁸, -carbocyclyl optionally substituted with 1-4 R⁴⁹, and —CN;
with the proviso that when A¹² is —C(R²)— and A¹³ is —C(R³)—, then R¹, R², R³, R⁴, and R⁵ are not all H;
with the proviso that when A¹² is N, then R¹, R³, R⁴, and R⁵ are not all H;
each R⁶ is independently selected from the group consisting of H, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
R⁷ is selected from the group consisting of unsubstituted —(C₁₋₉ alkyl), unsubstituted —(C₃₋₉ alkenyl), unsubstituted —(C₃₋₉ alkynyl), unsubstituted —(C₁₋₉ haloalkyl), —(C₁₋₄ alkylene)C(═O)OR¹¹, —(C₁₋₄ alkylene)C(═O)N(R¹²)₂, —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein;
each R¹⁰ is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
R¹¹ is selected from the group consisting of H and unsubstituted —(C₁₋₉ alkyl);
each R¹² is independently selected from the group consisting of H and substituted or unsubstituted —(C₁₋₉ alkyl);
each R¹³ is independently selected from the group consisting of halide, unsubstituted —(C₁₋₉ alkyl), unsubstituted —(C₁₋₉ haloalkyl), and —OR¹⁰;
each R¹⁴ is independently selected from the group consisting of unsubstituted —(C₁₋₉ alkyl), unsubstituted —(C₁₋₉ haloalkyl), and —OR¹⁰;
each R¹⁵ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁶ is independently selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
each R⁴⁸ is independently selected from the group consisting of unsubstituted —(C₁₋₉ alkyl), unsubstituted —(C₃₋₉ alkenyl), unsubstituted —(C₃₋₉ alkynyl), and unsubstituted —(C₁₋₉ haloalkyl);
each R⁴⁹ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
A¹ is selected from the group consisting of —C(R¹⁵)₂—, O, and S;
A² is selected from the group consisting of O and S;
A³ is selected from the group consisting of —C(R¹⁶)₂—, O and S;
each A⁴ is independently selected from the group consisting of —C(R¹⁶)₂—, O and S;
with the proviso that no more than two of A³ and/or A⁴ are O or S and that two O or S are never adjacent;
A¹² is selected from the group consisting of —C(R²)— and N;
A¹³ is selected from the group consisting of —C(R³)— and N;
n is 1 to 4; p is 0 to 1; and
with the proviso that Formula V is not a structure selected from the group consisting of:

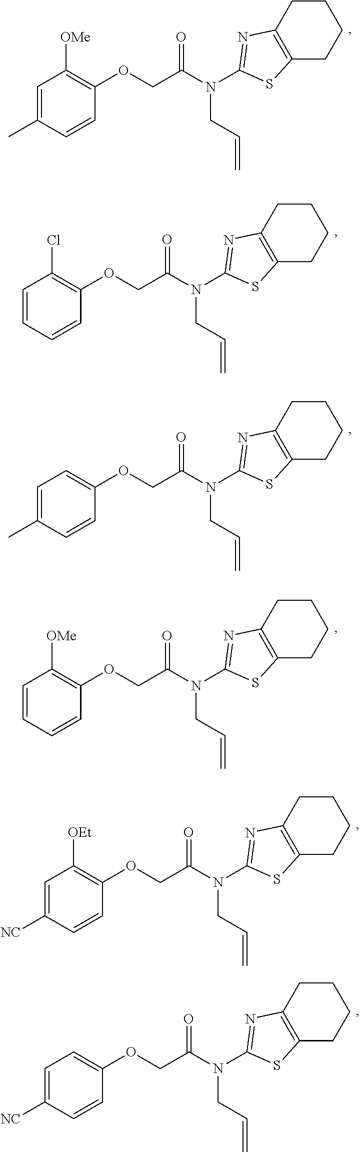 [SMILES: C=CCN(C(=O)COc1ccc(C)cc1OC)c1nc2c(s1)CCCC2 ; C=CCN(C(=O)COc1ccccc1Cl)c1nc2c(s1)CCCC2 ; C=CCN(C(=O)COc1ccc(C)cc1)c1nc2c(s1)CCCC2 ; C=CCN(C(=O)COc1ccccc1OC)c1nc2c(s1)CCCC2 ; C=CCN(C(=O)COc1ccc(C#N)cc1OCC)c1nc2c(s1)CCCC2 ; C=CCN(C(=O)COc1ccc(C#N)cc1)c1nc2c(s1)CCCC2]

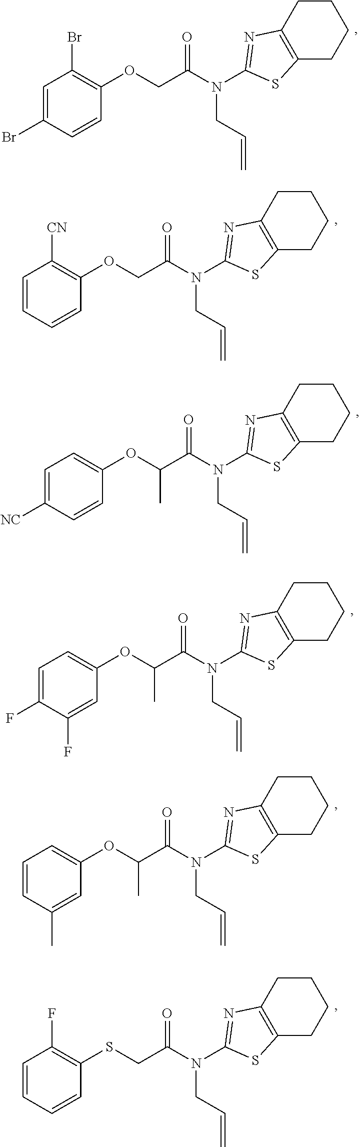

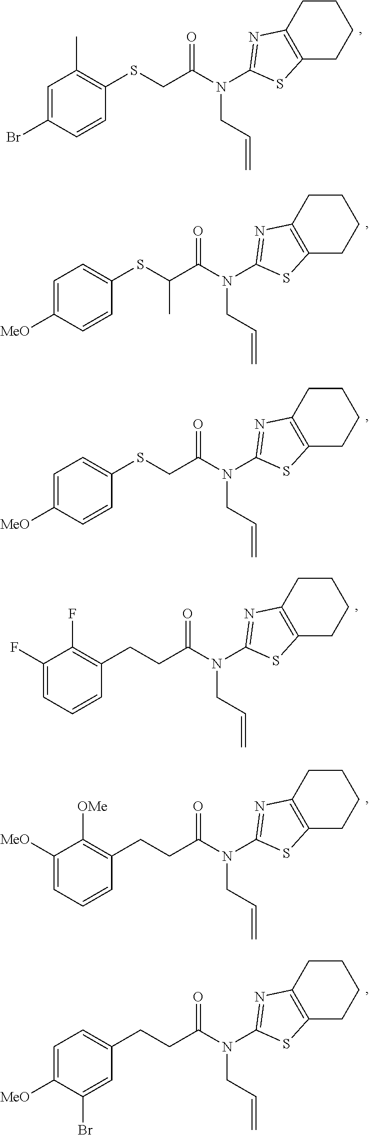 [SMILES: C=CCN(C(=O)CSc1ccc(Br)cc1C)c1nc2c(s1)CCCC2 ; C=CCN(C(=O)C(C)Sc1ccc(OC)cc1)c1nc2c(s1)CCCC2 ; C=CCN(C(=O)CSc1ccc(OC)cc1)c1nc2c(s1)CCCC2 ; C=CCN(C(=O)CCc1cccc(F)c1F)c1nc2c(s1)CCCC2 ; C=CCN(C(=O)CCc1cccc(OC)c1OC)c1nc2c(s1)CCCC2 ; C=CCN(C(=O)CCc1ccc(OC)c(Br)c1)c1nc2c(s1)CCCC2]

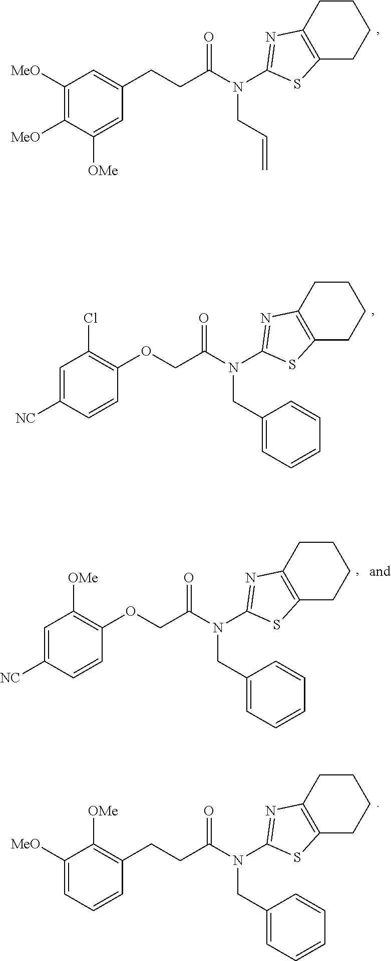 [SMILES: C=CCN(C(=O)CCc1cc(OC)c(OC)c(OC)c1)c1nc2c(s1)CCCC2 ; N#Cc1ccc(OCC(=O)N(Cc2ccccc2)c2nc3c(s2)CCCC3)c(Cl)c1 ; COc1cc(C#N)ccc1OCC(=O)N(Cc1ccccc1)c1nc2c(s1)CCCC2 ; COc1cccc(CCC(=O)N(Cc2ccccc2)c2nc3c(s2)CCCC3)c1OC]

In some embodiments, provided herein is a compound, or a pharmaceutically acceptable salt thereof, of Formula V:

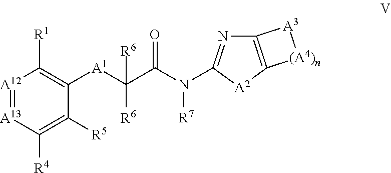

wherein:
R¹ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR⁴⁸, -carbocyclyl optionally substituted with 1-4 R⁴⁹, and —CN;
R² is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR⁴⁸, -carbocyclyl optionally substituted with 1-4 R⁴⁹, and —CN;
R³ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR⁴⁸, -carbocyclyl optionally substituted with 1-4 R⁴⁹, and —CN;
R⁴ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR⁴⁸, -carbocyclyl optionally substituted with 1-4 R⁴⁹, and —CN;
R⁵ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR⁴⁸, -carbocyclyl optionally substituted with 1-4 R⁴⁹, and —CN;
each R⁶ is independently selected from the group consisting of H, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
R⁷ is selected from the group consisting of unsubstituted —(C₁₋₉ alkyl), unsubstituted —(C₁₋₉ haloalkyl), —(C₁₋₄ alkylene)C(═O)OR¹¹, —(C₁₋₄ alkylene)C(═O)N(R¹²)₂, —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein;
each R¹⁰ is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
R¹¹ is selected from the group consisting of H and unsubstituted —(C₁₋₉ alkyl);
each R¹² is independently selected from the group consisting of H and substituted or unsubstituted —(C₁₋₉ alkyl);
each R¹³ is independently selected from the group consisting of halide, unsubstituted —(C₁₋₉ alkyl), unsubstituted —(C₁₋₉ haloalkyl), and —OR¹⁰;
each R¹⁴ is independently selected from the group consisting of unsubstituted —(C₁₋₉ alkyl), unsubstituted —(C₁₋₉ haloalkyl), and —OR¹⁰;
each R¹⁵ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁶ is independently selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
each R⁴⁸ is independently selected from the group consisting of unsubstituted —(C₁₋₉ alkyl), unsubstituted —(C₃₋₉ alkenyl), unsubstituted —(C₃₋₉ alkynyl), and unsubstituted —(C₁₋₉ haloalkyl);
each R⁴⁹ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
A¹ is selected from the group consisting of —C(R¹⁵)₂—, O, and S;
A² is selected from the group consisting of O and S;
A³ is selected from the group consisting of —C(R¹⁶)₂—, O and S;
each A⁴ is independently selected from the group consisting of —C(R¹⁶)₂—, O and S;
with the proviso that no more than two of A³ and/or A⁴ are O or S and that two O or S are never adjacent;
A¹² is selected from the group consisting of —C(R²)— and N;
A¹³ is selected from the group consisting of —C(R³)— and N;
n is 1, 2, 3, or 4; and
p is 0 or 1.

For example, provided herein is a compound, or a pharmaceutically acceptable salt thereof, of Formula V:

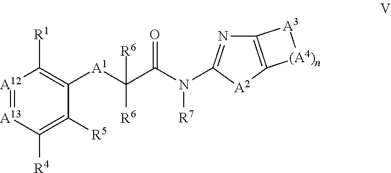

wherein:
R¹ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR⁴⁸, -carbocyclyl optionally substituted with 1-4 R⁴⁹, and —CN;
R² is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR⁴⁸, -carbocyclyl optionally substituted with 1-4 R⁴⁹, and —CN;
R³ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR⁴⁸, -carbocyclyl optionally substituted with 1-4 R⁴⁹, and —CN;
R⁴ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR⁴⁸, -carbocyclyl optionally substituted with 1-4 R⁴⁹, and —CN;
R⁵ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR⁴⁸, -carbocyclyl optionally substituted with 1-4 R⁴⁹, and —CN;
with the proviso that when A¹² is —C(R²)— and A¹³ is —C(R³)—, then R¹, R², R³, R⁴, and R⁵ are not all H;
with the proviso that when A¹² is N, then R¹, R³, R⁴, and R⁵ are not all H;
each R⁶ is independently selected from the group consisting of H, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
R⁷ is selected from the group consisting of unsubstituted —(C₁₋₉ alkyl), unsubstituted —(C₁₋₉ haloalkyl), —(C₁₋₄ alkylene)C(═O)OR¹¹, —(C₁₋₄ alkylene)C(═O)N(R¹²)₂, —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein;
with the proviso that when R⁷ is —(CH₂)aryl, then the aryl is not unsubstituted or substituted with halide or methyl;
each R¹⁰ is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
R¹¹ is selected from the group consisting of H and unsubstituted —(C₁₋₉ alkyl);
each R¹² is independently selected from the group consisting of H and substituted or unsubstituted —(C₁₋₉ alkyl);
each R¹³ is independently selected from the group consisting of halide, unsubstituted —(C₁₋₉ alkyl), unsubstituted —(C₁₋₉ haloalkyl), and —OR¹⁰;
each R¹⁴ is independently selected from the group consisting of unsubstituted —(C₁₋₉ alkyl), unsubstituted —(C₁₋₉ haloalkyl), and —OR¹⁰;
each R¹⁵ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁶ is independently selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
each R⁴⁸ is independently selected from the group consisting of unsubstituted —(C₁₋₉ alkyl), unsubstituted —(C₃₋₉ alkenyl), unsubstituted —(C₃₋₉ alkynyl), and unsubstituted —(C₁₋₉ haloalkyl);
each R⁴⁹ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
A¹ is selected from the group consisting of —C(R¹⁵)₂—, O, and S;
A² is selected from the group consisting of O and S;
A³ is selected from the group consisting of —C(R¹⁶)₂—, O and S;
each A⁴ is independently selected from the group consisting of —C(R¹⁶)₂—, O and S;
with the proviso that no more than two of A³ and/or A⁴ are O or S and that two O or S are never adjacent;
A¹² is selected from the group consisting of —C(R²)— and N;
A¹³ is selected from the group consisting of —C(R³)— and N;
n is 1, 2, 3, or 4; and
p is 0 or 1.

Some embodiments of the present disclosure include compounds of Formula VI:

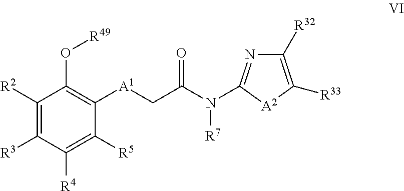

or enantiomers, diastereomers, tautomers, prodrugs, and pharmaceutically acceptable salts thereof.

In some embodiments of Formula VI, R², R³, R⁴, and R⁵ are independently selected from the group consisting of H, halide (e.g., F, Cl, Br, I), unsubstituted —(C₁₋₉ alkyl) (e.g., C₁₋₈, C₁₋₇, C₁₋₆, C₁₋₅, C₁₋₄, C₁₋₃, C₁₋₂, C₁), unsubstituted —(C₂₋₉ alkenyl) (e.g., C₂₋₈, C₂₋₇, C₂₋₆, C₂₋₅, C₂₋₄, C₂₋₃, C₂), unsubstituted —(C₂₋₉ alkynyl) (e.g., C₂₋₈, C₂₋₇, C₂₋₆, C₂₋₅, C₂₋₄, C₂₋₃, C₂), unsubstituted —(C₁₋₉ haloalkyl) (e.g., C₁₋₈, C₁₋₇, C₁₋₆, C₁₋₅, C₁₋₄, C₁₋₃, C₁₋₂, C₁), and —CN;

In some embodiments of Formula VI, R², R³, R⁴, and R⁵ are independently selected from the group consisting of H, F, Cl, Br, unsubstituted —(C₁₋₄ alkyl) (e.g., C₁₋₃, C₁₋₂, C₁), unsubstituted —(C₂₋₄ alkenyl) (e.g., C₂₋₃, C₂), unsubstituted —(C₂₋₄ alkynyl) (e.g., C₂₋₃, C₂), unsubstituted —(C₁₋₄ haloalkyl) (e.g., C₁₋₃, C₁₋₂, C₁), and —CN.

In some embodiments of Formula VI, R², R³, R⁴, and R⁵ are independently selected from the group consisting of F, Cl, unsubstituted —(C₁₋₂ alkyl) (e.g., Me), unsubstituted —(C₂ alkenyl), unsubstituted —(C₂ alkynyl), unsubstituted —(C₁₋₂ haloalkyl) (e.g., C₁), and —CN.

In some embodiments of Formula VI, R², R³, R⁴, and R⁵ are independently selected from the group consisting of F, Cl, Me, —CF₃, and —CN.

In some embodiments of Formula VI, R³² and R³³ are independently selected from the group consisting of H, unsubstituted —(C₁₋₉ alkyl) (e.g., C₁₋₈, C₁₋₇, C₁₋₆, C₁₋₅, C₁₋₄, C₁₋₃, C₁₋₂, C₁), unsubstituted —(C₂₋₉ alkenyl) (e.g., C₂₋₈, C₂₋₇, C₂₋₆, C₂₋₅, C₂₋₄, C₂₋₃, C₂), unsubstituted —(C₂₋₉ alkynyl) (e.g., C₂₋₈, C₂₋₇, C₂₋₆, C₂₋₅, C₂₋₄, C₂₋₃, C₂), unsubstituted —(C₁₋₉ fluoroalkyl) (e.g., C₁₋₈, C₁₋₇, C₁₋₆, C₁₋₅, C₁₋₄, C₁₋₃, C₁₋₂, C₁), and —CN.

In some embodiments of Formula VI, R³² and R³³ are independently selected from the group consisting of H, unsubstituted —(C₁₋₄ alkyl) (e.g., C₁₋₃, C₁₋₂, C₁), unsubstituted —(C₂₋₄ alkenyl) (e.g., C₂₋₃, C₂), unsubstituted —(C₂₋₄ alkynyl) (e.g., C₂₋₃, C₂), unsubstituted —(C₁₋₄ fluoroalkyl) (e.g., C₁₋₃, C₁₋₂, C₁), and —CN.

In some embodiments of Formula VI, R³² and R³³ are independently selected from the group consisting of H, unsubstituted —(C₁₋₂ alkyl) (e.g., Me), unsubstituted —(C₂ alkenyl), unsubstituted —(C₂ alkynyl), unsubstituted —(C₁₋₂ fluoroalkyl) (e.g., C₁), and —CN.

In some embodiments of Formula VI, R³² and R³³ are independently selected from the group consisting of H, Me, —CF₃, and —CN.

In some embodiments of Formula VI, R⁴⁹ is selected from the group consisting of unsubstituted —(C₃₋₉ alkyl) (e.g., C₃₋₈, C₃₋₇, C₃₋₆, C₃₋₅, C₃₋₄, C₃), unsubstituted —(C₃₋₉ alkenyl) (e.g., C₃₋₈, C₃₋₇, C₃₋₆, C₃₋₅, C₃₋₄, C₃), unsubstituted —(C₃₋₉ alkynyl) (e.g., C₃₋₈, C₃₋₇, C₃₋₆, C₃₋₅, C₃₋₄, C₃), unsubstituted —(C₃₋₉ haloalkyl) (e.g., C₃₋₈, C₃₋₇, C₃₋₆, C₃₋₅, C₃₋₄, C₃), —(C₁₋₄ alkylene)ₚheteroaryl optionally substituted with 1-4 (e.g., 1-3, 1-2, 1) R⁵⁰, and —(C₁₋₄ alkylene)ₚaryl optionally substituted with 1-5 (e.g., 1-5, 1-3, 1-2, 1) R⁵¹, and —(C₁₋₄ alkylene)ₚcarbocyclyl optionally substituted with 1-10 (e.g., 1-9, 1-8, 1-7, 1-6, 1-5, 1-4, 1-3, 1-2, 1) R⁵²; wherein each —(C₁₋₄ alkylene) is, independently, optionally substituted with one or more substituents as defined herein.

In some embodiments of Formula VI, R⁴⁹ is selected from the group consisting of unsubstituted —(C₃₋₅ alkyl) (e.g., C₃₋₄, C₃), unsubstituted —(C₃₋₅ alkenyl) (e.g., C₃₋₄, C₃), unsubstituted —(C₃₋₅ alkynyl) (e.g., C₃₋₄, C₃), unsubstituted —(C₃₋₅ haloalkyl) (e.g., C₃₋₄, C₃), —(CH₂)heteroaryl optionally substituted with 1-4 (e.g., 1-3, 1-2, 1) R⁵⁰, and —(CH₂)aryl optionally substituted with 1-5 (e.g., 1-4, 1-3, 1-2, 1) R⁵¹, and —(CH₂)carbocyclyl optionally substituted with 1-10 (e.g., 1-9, 1-8, 1-7, 1-6, 1-5, 1-4, 1-3, 1-2, 1) R⁵².

In some embodiments of Formula VI, R⁴⁹ is selected from the group consisting of unsubstituted —(C₃₋₅ alkyl) (e.g., C₃₋₄, C₃) and —(CH₂)aryl optionally substituted with 1-5 (e.g., 1-4, 1-3, 1-2, 1) R⁵¹.

In some embodiments of Formula VI, there is the proviso that when R⁷ is H, then R⁴⁹ is not —(C₃ alkyl).

In some embodiments of Formula VI, each R⁵⁰ is independently selected from the group consisting of halide (e.g., F, Cl, Br, I), unsubstituted —(C₁₋₉ alkyl) (e.g., C₁₋₈, C₁₋₇, C₁₋₆, C₁₋₅, C₁₋₄, C₁₋₃, C₁₋₂, C₁), unsubstituted —(C₁₋₉ haloalkyl) (e.g., C₁₋₈, C₁₋₇, C₁₋₆, C₁₋₅, C₁₋₄, C₁₋₃, C₁₋₂, C₁), and —OR⁰.

In some embodiments of Formula VI, each R⁵¹ is independently selected from the group consisting of F, Cl, Br, unsubstituted —(C₁₋₄ alkyl) (e.g., C₁₋₃, C₁₋₂, C₁), unsubstituted —(C₁₋₄ haloalkyl) (e.g., C₁₋₃, C₁₋₂, C₁), and —O(C₁₋₄ alkyl) (e.g., C₁₋₃, C₁₋₂, C₁).

In some embodiments of Formula VI, each R⁵¹ is independently selected from the group consisting of F, Cl, unsubstituted —(C₁₋₂ alkyl) (e.g., Me), unsubstituted —(C₁₋₂ haloalkyl) (e.g., C₁), and —O(C₁₋₂ alkyl) (e.g., Me).

In some embodiments of Formula VI, each R⁵¹ is independently selected from the group consisting of F, Cl, Me, —CF₃, and —OMe.

In some embodiments of Formula VI, each R⁵¹ is independently selected from the group consisting of halide (e.g., F, Cl, Br, I), unsubstituted —(C₁₋₉ alkyl) (e.g., C₁₋₈, C₁₋₇, C₁₋₆, C₁₋₅, C₁₋₄, C₁₋₃, C₁₋₂, C₁), unsubstituted —(C₁₋₉ haloalkyl) (e.g., C₁₋₈, C₁₋₇, C₁₋₆, C₁₋₅, C₁₋₄, C₁₋₃, C₁₋₂, C₁), and —OR⁰.

In some embodiments of Formula VI, each R⁵¹ is independently selected from the group consisting of F, Cl, Br, unsubstituted —(C₁₋₄ alkyl) (e.g., C₁₋₃, C₁₋₂, C₁), unsubstituted —(C₁₋₄ haloalkyl) (e.g., C₁₋₃, C₁₋₂, C₁), and —O(C₁₋₄ alkyl) (e.g., C₁₋₃, C₁₋₂, C₁).

In some embodiments of Formula VI, each R⁵¹ is independently selected from the group consisting of F, Cl, unsubstituted —(C₁₋₂ alkyl) (e.g., Me), unsubstituted —(C₁₋₂ haloalkyl) (e.g., C₁), and —O(C₁₋₂ alkyl) (e.g., Me).

In some embodiments of Formula VI, each R⁵¹ is independently selected from the group consisting of F, Cl, Me, —CF₃, and —OMe.

In some embodiments of Formula VI, each R⁵² is selected from the group consisting of halide (e.g., F, Cl, Br, I), unsubstituted —(C₁₋₉ alkyl) (e.g., C₁₋₈, C₁₋₇, C₁₋₆, C₁₋₅, C₁₋₄, C₁₋₃, C₁₋₂, C₁), and unsubstituted —(C₁₋₉ haloalkyl) (e.g., C₁₋₈, C₁₋₇, C₁₋₆, C₁₋₅, C₁₋₄, C₁₋₃, C₁₋₂, C₁).

In some embodiments of Formula VI, each R⁵² is selected from the group consisting of F, Cl, Br, unsubstituted —(C₁₋₄ alkyl) (e.g., C₁₋₃, C₁₋₂, C₁), and unsubstituted —(C₁₋₄ haloalkyl) (e.g., C₁₋₃, C₁₋₂, C₁).

In some embodiments of Formula VI, each R⁵² is selected from the group consisting of F, Cl, unsubstituted —(C₁₋₂ alkyl) (e.g., Me), and unsubstituted —(C₁₋₂ haloalkyl) (e.g., C₁).

In some embodiments of Formula VI, each R⁵² is selected from the group consisting of F, Cl, Me, and —CF₃.

In some embodiments of Formula VI, R⁵⁰, R″ and R⁵² are independently selected from the group consisting of F, Cl, Br, and I.

In some embodiments, provided herein is a compound, or a pharmaceutically acceptable salt thereof, of Formula VI:

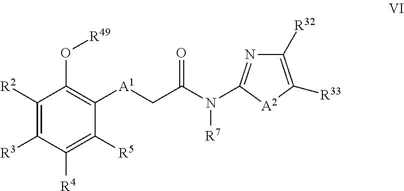

wherein:

R² is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R³ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R⁴ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R⁵ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R⁷ is selected from the group consisting of H, unsubstituted —(C₁₋₉ alkyl), unsubstituted —(C₃₋₉ alkenyl), unsubstituted —(C₃₋₉ alkynyl), unsubstituted —(C₁₋₉ haloalkyl), —(C₁₋₄ alkylene)C(═O)OR¹¹, —(C₁₋₄ alkylene)C(═O)N(R¹²)₂, —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein;
each R¹⁰ is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
R¹¹ is selected from the group consisting of H and unsubstituted —(C₁₋₉ alkyl);
each R¹² is independently selected from the group consisting of H and substituted or unsubstituted —(C₁₋₉ alkyl);
each R¹³ is independently selected from the group consisting of halide, unsubstituted —(C₁₋₉ alkyl), unsubstituted —(C₁₋₉ haloalkyl), and —OR¹⁰;
each R¹⁴ is independently selected from the group consisting of halide, unsubstituted —(C₁₋₉ alkyl), unsubstituted —(C₁₋₉ haloalkyl), and —OR¹⁰;
each R¹⁵ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
R³² is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ fluoroalkyl), and —CN;
R³³ is selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R⁴⁹ is selected from the group consisting of unsubstituted —(C₃₋₉ alkyl), unsubstituted —(C₃₋₉ alkenyl), unsubstituted —(C₃₋₉ alkynyl), unsubstituted —(C₃₋₉ haloalkyl), —(C₁₋₄ alkylene)ₚheteroaryl optionally substituted with 1-4 R⁵⁰, and —(C₁₋₄ alkylene)ₚaryl optionally substituted with 1-5 R⁵¹, and —(C₁₋₄ alkylene)ₚcarbocyclyl optionally substituted with 1-10 R⁵²; wherein each —(C₁₋₄ alkylene) is, independently, optionally substituted with one or more substituents as defined herein;
each R⁵⁰ is independently selected from the group consisting of halide, unsubstituted —(C₁₋₉ alkyl), unsubstituted —(C₁₋₉ haloalkyl), and —OR¹⁰;
each R⁵¹ is independently selected from the group consisting of halide, unsubstituted —(C₁₋₉ alkyl), unsubstituted —(C₁₋₉ haloalkyl), and —OR¹⁰;
each R⁵² is independently selected from the group consisting of halide, unsubstituted —(C₁₋₉ alkyl), and unsubstituted —(C₁₋₉ haloalkyl);
A¹ is selected from the group consisting of —C(R¹⁵)₂—, O, and S;
A² is selected from the group consisting of O and S; and
each p independently is 0 or 1.

For example, provided herein is a compound, or a pharmaceutically acceptable salt thereof, of Formula VI:

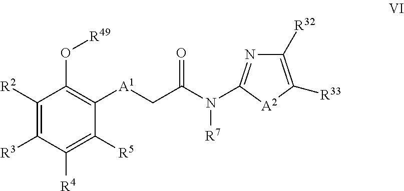

wherein:

R² is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R³ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R⁴ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R⁵ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R⁷ is selected from the group consisting of H, unsubstituted —(C₁₋₉ alkyl), unsubstituted —(C₃₋₉ alkenyl), unsubstituted —(C₃₋₉ alkynyl), unsubstituted —(C₁₋₉ haloalkyl), —(C₁₋₄ alkylene)C(═O)OR¹¹, —(C₁₋₄ alkylene)C(═O)N(R¹²)₂, —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein;
each R¹⁰ is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
R¹¹ is selected from the group consisting of H and unsubstituted —(C₁₋₉ alkyl);
each R¹² is independently selected from the group consisting of H and substituted or unsubstituted —(C₁₋₉ alkyl);
each R¹³ is independently selected from the group consisting of halide, unsubstituted —(C₁₋₉ alkyl), unsubstituted —(C₁₋₉ haloalkyl), and —OR¹⁰;
each R¹⁴ is independently selected from the group consisting of halide, unsubstituted —(C₁₋₉ alkyl), unsubstituted —(C₁₋₉ haloalkyl), and —OR¹⁰;
each R¹⁵ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
R³² is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ fluoroalkyl), and —CN;
R³³ is selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R⁴⁹ is selected from the group consisting of unsubstituted —(C₃₋₉ alkyl), unsubstituted —(C₃₋₉ alkenyl), unsubstituted —(C₃₋₉ alkynyl), unsubstituted —(C₃₋₉ haloalkyl), —(C₁₋₄ alkylene)ₚheteroaryl optionally substituted with 1-4 R⁵⁰, and —(C₁₋₄ alkylene)ₚaryl optionally substituted with 1-5 R⁵¹, and —(C₁₋₄ alkylene)ₚcarbocyclyl optionally substituted with 1-10 R⁵²; wherein each —(C₁₋₄ alkylene) is, independently, optionally substituted with one or more substituents as defined herein;
each R⁵⁰ is independently selected from the group consisting of halide, unsubstituted —(C₁₋₉ alkyl), unsubstituted —(C₁₋₉ haloalkyl), and —OR¹⁰;
each R⁵¹ is independently selected from the group consisting of halide, unsubstituted —(C₁₋₉ alkyl), unsubstituted —(C₁₋₉ haloalkyl), and —OR¹⁰;
each R⁵² is independently selected from the group consisting of halide, unsubstituted —(C₁₋₉ alkyl), and unsubstituted —(C₁₋₉ haloalkyl);
A¹ is selected from the group consisting of —C(R¹⁵)₂—, O, and S;
A² is selected from the group consisting of O and S;
each p independently is 0 or 1; and
with the proviso that Formula VI is not:

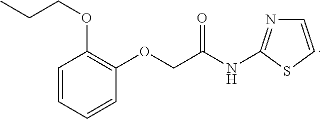 [SMILES: CCCOc1ccccc1OCC(=O)Nc1nccs1]

In some embodiments, provided herein is a compound, or a pharmaceutically acceptable salt thereof, of Formula VI:

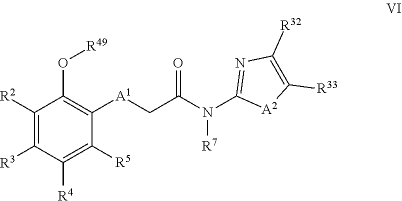

wherein:

R² is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R³ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R⁴ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R⁵ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R⁷ is selected from the group consisting of H, unsubstituted —(C₁₋₉ alkyl), unsubstituted —(C₃₋₉ alkenyl), unsubstituted —(C₃₋₉ alkynyl), unsubstituted —(C₁₋₉ haloalkyl), —(C₁₋₄ alkylene)C(═O)OR¹¹, —(C₁₋₄ alkylene)C(═O)N(R¹²)₂, —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein;
each R¹⁰ is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
R¹¹ is selected from the group consisting of H and unsubstituted —(C₁₋₉ alkyl);
each R¹² is independently selected from the group consisting of H and substituted or unsubstituted —(C₁₋₉ alkyl);
each R¹³ is independently selected from the group consisting of halide, unsubstituted —(C₁₋₉ alkyl), unsubstituted —(C₁₋₉ haloalkyl), and —OR¹⁰;
each R¹⁴ is independently selected from the group consisting of halide, unsubstituted —(C₁₋₉ alkyl), unsubstituted —(C₁₋₉ haloalkyl), and —OR¹⁰;
each R¹⁵ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
R³² is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ fluoroalkyl), and —CN;
R³³ is selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R⁴⁹ is selected from the group consisting of unsubstituted —(C₃₋₉ alkyl), unsubstituted —(C₃₋₉ alkenyl), unsubstituted —(C₃₋₉ alkynyl), unsubstituted —(C₃₋₉ haloalkyl), —(C₁₋₄ alkylene)ₚheteroaryl optionally substituted with 1-4 R⁵⁰, and —(C₁₋₄ alkylene)ₚaryl optionally substituted with 1-5 R⁵¹, and —(C₁₋₄ alkylene)ₚcarbocyclyl optionally substituted with 1-10 R⁵²; wherein each —(C₁₋₄ alkylene) is, independently, optionally substituted with one or more substituents as defined herein;
with the proviso that when R⁷ is H, then R⁴⁹ is not —(C₃ alkyl);
each R⁵⁰ is independently selected from the group consisting of halide, unsubstituted —(C₁₋₉ alkyl), unsubstituted —(C₁₋₉ haloalkyl), and —OR¹⁰;
each R⁵¹ is independently selected from the group consisting of halide, unsubstituted —(C₁₋₉ alkyl), unsubstituted —(C₁₋₉ haloalkyl), and —OR¹⁰;
each R⁵² is selected from the group consisting of halide, unsubstituted —(C₁₋₉ alkyl), and unsubstituted —(C₁₋₉ haloalkyl);
A¹ is selected from the group consisting of —C(R¹⁵)₂—, O, and S;
A² is selected from the group consisting of O and S; and
each p independently is 0 or 1.

For example, provided herein is a compound, or a pharmaceutically acceptable salt thereof, of Formula VI:

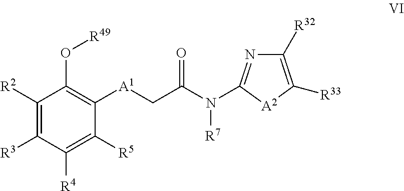

wherein:

R² is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R³ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R⁴ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R⁵ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R⁷ is selected from the group consisting of H, unsubstituted —(C₁₋₉ alkyl), unsubstituted —(C₃₋₉ alkenyl), unsubstituted —(C₃₋₉ alkynyl), unsubstituted —(C₁₋₉ haloalkyl), —(C₁₋₄ alkylene)C(═O)OR¹¹, —(C₁₋₄ alkylene)C(═O)N(R¹²)₂, —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein;
each R¹⁰ is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
R¹¹ is selected from the group consisting of H and unsubstituted —(C₁₋₉ alkyl);
each R¹² is independently selected from the group consisting of H and substituted or unsubstituted —(C₁₋₉ alkyl);
each R¹³ is independently selected from the group consisting of halide, unsubstituted —(C₁₋₉ alkyl), unsubstituted —(C₁₋₉ haloalkyl), and —OR¹⁰;
each R¹⁴ is independently selected from the group consisting of halide, unsubstituted —(C₁₋₉ alkyl), unsubstituted —(C₁₋₉ haloalkyl), and —OR¹⁰;
each R¹⁵ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
R³² is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ fluoroalkyl), and —CN;
R³³ is selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R⁴⁹ is selected from the group consisting of unsubstituted —(C₃₋₉ alkyl), unsubstituted —(C₃₋₉ alkenyl), unsubstituted —(C₃₋₉ alkynyl), unsubstituted —(C₃₋₉ haloalkyl), —(C₁₋₄ alkylene)ₚheteroaryl optionally substituted with 1-4 R⁵⁰, and —(C₁₋₄ alkylene)ₚaryl optionally substituted with 1-5 R⁵¹, and —(C₁₋₄ alkylene)ₚcarbocyclyl optionally substituted with 1-10 R⁵²; wherein each —(C₁₋₄ alkylene) is, independently, optionally substituted with one or more substituents as defined herein;
with the proviso that when R⁷ is H, then R⁴⁹ is not —(C₃ alkyl);
each R⁵⁰ is independently selected from the group consisting of halide, unsubstituted —(C₁₋₉ alkyl), unsubstituted —(C₁₋₉ haloalkyl), and —OR¹⁰;
each R⁵¹ is independently selected from the group consisting of halide, unsubstituted —(C₁₋₉ alkyl), unsubstituted —(C₁₋₉ haloalkyl), and —OR¹⁰;
each R⁵² is selected from the group consisting of halide, unsubstituted —(C₁₋₉ alkyl), and unsubstituted —(C₁₋₉ haloalkyl);
A¹ is selected from the group consisting of —C(R¹⁵)₂—, O, and S;
A² is selected from the group consisting of O and S; and
each p independently is 0 or 1.

In some embodiments of Formulas I, II, III, IV, V, and VI, each —(C₁₋₄ alkylene) is —(C₁₋₃ alkylene).

In some embodiments of Formulas I, II, III, IV, V, and VI, each —(C₁₋₄ alkylene) is —(C₁₋₂ alkylene).

In some embodiments of Formulas I, II, III, IV, V, and VI, each —(C₁₋₄ alkylene) is —(C₁ alkylene).

In some embodiments of Formulas I, II, III, IV, V, and VI, each —(C₁₋₄ alkylene) is —CH₂—.

In some embodiments of Formulas I, II, III, IV, V, and VI, each —(C₁₋₄ alkylene) is optionally substituted with halide (e.g., F, Cl, Br, I).

In some embodiments of Formulas I, II, III, IV, V, and VI, each —(C₁₋₄ alkylene) is optionally substituted with F.

In some embodiments of Formulas I and V,

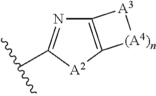

is selected from the group consisting of

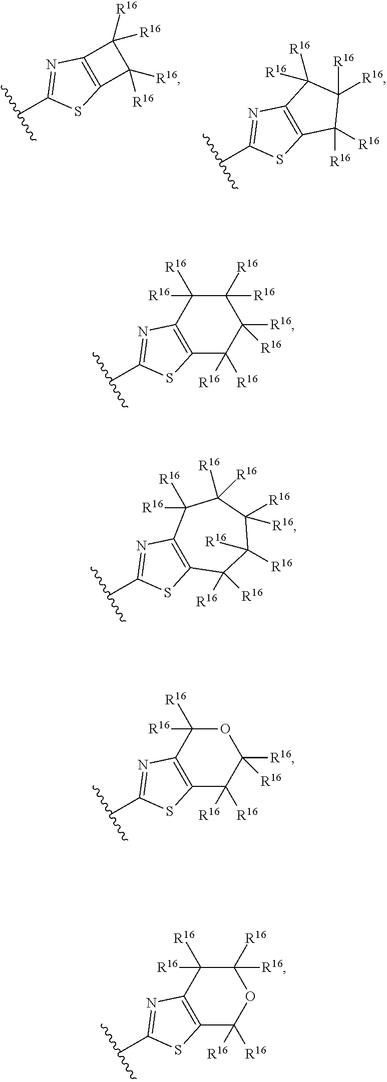

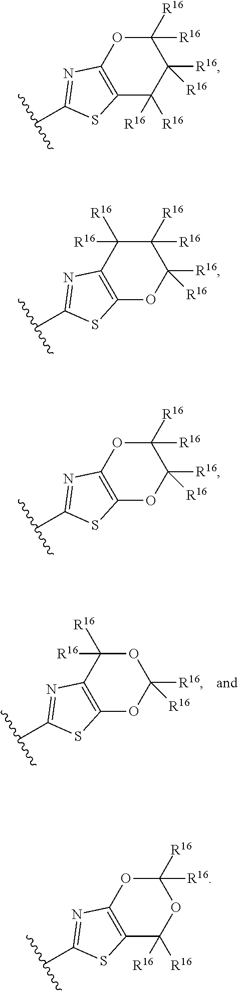

In some embodiments of Formulas I and V,

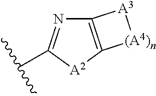

is selected from the group consisting of

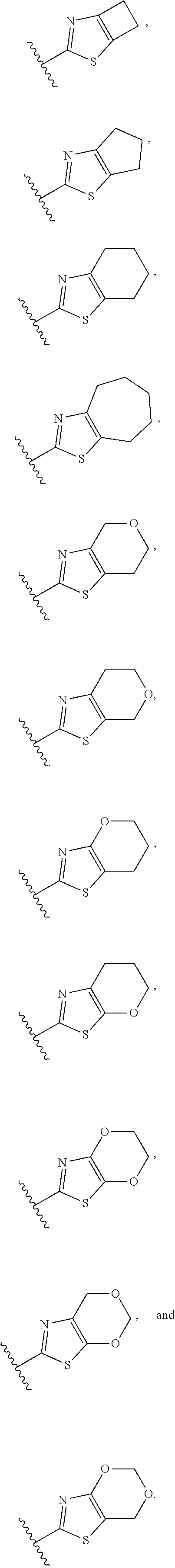

In some embodiments of Formulas I and V,

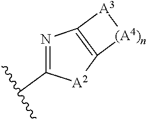

is selected from the group consisting of

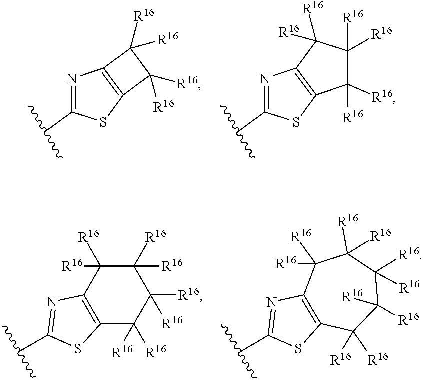

In some embodiments of Formulas I and V,

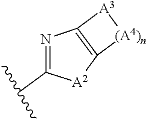

is selected from the group consisting of

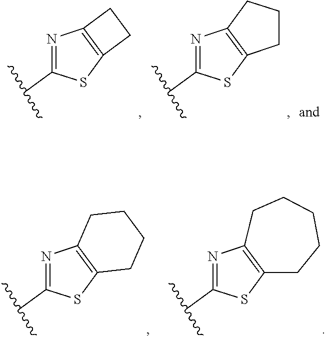 [SMILES: *c1nc2c(s1)CC2 ; *c1nc2c(s1)CCC2 ; *c1nc2c(s1)CCCC2 ; *c1nc2c(s1)CCCCC2]

In some embodiments of Formulas I and V,

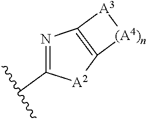

is selected from the group consisting of

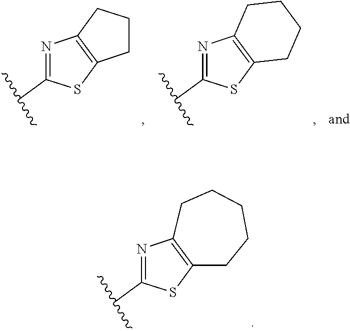 [SMILES: *c1nc2c(s1)CCC2 ; *c1nc2c(s1)CCCC2 ; *c1nc2c(s1)CCCCC2]

In some embodiments of Formulas I and V,

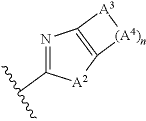

is selected from the group consisting of

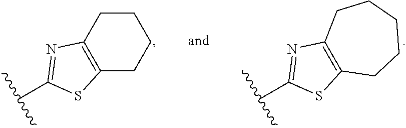 [SMILES: *c1nc2c(s1)CCCC2 ; *c1nc2c(s1)CCCCC2]

In some embodiments of Formulas I and V,

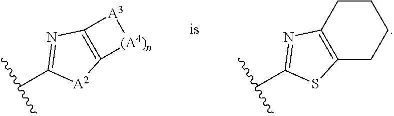

In some embodiments of Formula I,

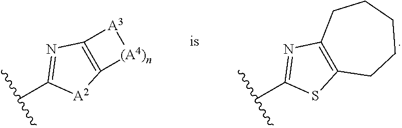

In some embodiments of Formulas I and V,

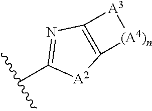

is selected from the group consisting of

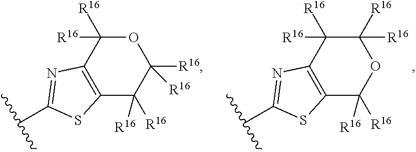

In some embodiments of Formulas I and V,

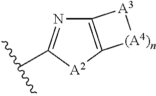

is selected from the group consisting of

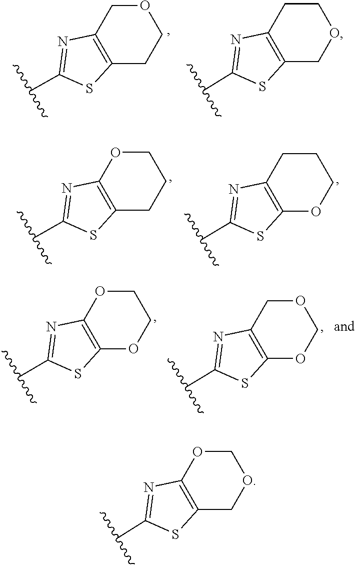

In some embodiments of Formulas I, II, III, and IV, R³, R⁴, and R⁵ are independently selected from the group consisting of H, halide (e.g., F, Cl, Br, I), unsubstituted —(C₁₋₆ alkyl) (e.g., C₁₋₅, C₁₋₄, C₁₋₃, C₁₋₂, C₁), unsubstituted —(C₁₋₄ haloalkyl) (e.g., C₁₋₃, C₁₋₂, C₁), —OR¹⁰, and —CN.

In some embodiments of Formulas I, II, III, and IV, R³, R⁴, and R⁵ are independently selected from the group consisting of H, F, Cl, Br, unsubstituted —(C₁₋₄ alkyl) (e.g., C₁₋₃, C₁₋₂, C₁), unsubstituted —(C₁₋₃ haloalkyl) (e.g., C₁₋₂, C₁), —OH, —O(C₁₋₄ alkyl) (e.g., C₁₋₃, C₁₋₂, C₁), and —CN.

In some embodiments of Formulas I, II, III, and IV, R³, R⁴, and R⁵ are independently selected from the group consisting of H, F, Cl, unsubstituted —(C₁₋₂ alkyl) (e.g., Me), unsubstituted —(C₁₋₂ haloalkyl) (e.g., C₁), —OH, —O(C₁₋₂ alkyl) (e.g., Me), and —CN.

In some embodiments of Formulas I, II, III, and IV, R³, R⁴, and R⁵ are independently selected from the group consisting of H, F, Cl, Me, —CF₃, —OH, —OMe, and —CN.

In some embodiments of Formulas I, II, III, and IV, R³, R⁴, and R⁵ are independently selected from the group consisting of H and halide (e.g., F, Cl, Br, I).

In some embodiments of Formulas I, II, III, and IV, R³, R⁴, and R⁵ are H.

In some embodiments of Formulas II, IV, and V, each R⁶ is independently selected from the group consisting of H, unsubstituted —(C₁₋₆ alkyl) (e.g., C₁₋₅, C₁₋₄, C₁₋₃, C₁₋₂, C₁), unsubstituted —(C₂₋₆ alkenyl) (e.g., C₂₋₈, C₂₋₄, C₂₋₃, C₂), unsubstituted —(C₂₋₆ alkynyl) (e.g., C₂₋₅, C₂₋₄, C₂₋₃, C₂), and unsubstituted —(C₁₋₆ haloalkyl) (e.g., C₁₋₈, C₁₋₄, C₁₋₃, C₁₋₂, C₁).

In some embodiments of Formulas I and III, each R⁶ is independently selected from the group consisting of H, unsubstituted —(C₁₋₆ alkyl) (e.g., C₁₋₅, C₁₋₄, C₁₋₃, C₁₋₂, C₁), unsubstituted —(C₂₋₆ alkenyl) (e.g., C₂₋₅, C₂₋₄, C₂₋₃, C₂), unsubstituted —(C₂₋₆ alkynyl) (e.g., C₂₋₅, C₂₋₄, C₂₋₃, C₂), unsubstituted —(C₁₋₆ haloalkyl) (e.g., C₁₋₅, C₁₋₄, C₁₋₃, C₁₋₂, C₁), —(C₁₋₄ alkylene)ₚheteroaryl optionally substituted with 1-4 (e.g., 1-3. 1-2. 1) R¹³, and —(C₁₋₄ alkylene)ₚaryl optionally substituted with 1-5 (e.g., 1-4, 1-3. 1-2. 1) R¹⁴; wherein each —(C₁₋₄ alkylene) is, independently, optionally substituted with one or more substituents as defined herein;

In some embodiments of Formulas II, IV, and V, each R⁶ is independently selected from the group consisting of H, unsubstituted —(C₁₋₂ alkyl) (e.g., Me), unsubstituted —(C₂ alkenyl), unsubstituted —(C₂ alkynyl), and unsubstituted —(C₁₋₂ haloalkyl) (e.g., C₁).

In some embodiments of Formulas I and III, each R⁶ is independently selected from the group consisting of H, unsubstituted —(C₁₋₂ alkyl) (e.g., Me), unsubstituted —(C₂ alkenyl), unsubstituted —(C₂ alkynyl), unsubstituted —(C₁₋₂ haloalkyl) (e.g., C₁), —(CH₂)heteroaryl optionally substituted with 1-2 (e.g., 1) R¹³, and —(CH₂)aryl optionally substituted with 1-2 (e.g., 1) R¹⁴.

In some embodiments of Formulas I, II, III, IV, and V, each R⁶ is independently selected from the group consisting of H, Me, and —CF₃.

In some embodiments of Formulas I, II, III, IV, V, and VI, R⁷ is selected from the group consisting of H, unsubstituted —(C₁₋₉ alkyl) (e.g., C₁₋₈, C₁₋₇, C₁₋₆, C₁₋₅, C₁₋₄, C₁₋₃, C₁₋₂, C₁), unsubstituted —(C₃₋₉ alkenyl) (e.g., C₃₋₈, C₃₋₇, C₃₋₆, C₃₋₅, C₃₋₄, C₃), unsubstituted —(C₃₋₉ alkynyl) (e.g., C₃₋₈, C₃₋₇, C₃₋₆, C₃₋₅, C₃₋₄, C₃), unsubstituted —(C₁₋₉ haloalkyl) (e.g., C₁₋₈, C₁₋₇, C₁₋₆, C₁₋₈, C₁₋₄, C₁₋₃, C₁₋₂, C₁), —(C₁₋₄ alkylene)C(═O)OR¹¹, —(C₁₋₄ alkylene)C(═O)N(R¹²)₂, —(C₁₋₄ alkylene)ₚheteroaryl optionally substituted with 1-4 (e.g., 1-3. 1-2. 1) R¹³, and —(C₁₋₄ alkylene)ₚaryl optionally substituted with 1-5 (e.g., 1-4, 1-3. 1-2. 1) R¹⁴; wherein each —(C₁₋₄ alkylene) is, independently, optionally substituted with one or more substituents as defined herein.

In some embodiments of Formulas I, II, III, IV, V, and VI, R⁷ is selected from the group consisting of H, unsubstituted —(C₁₋₄ alkyl) (e.g., C₁₋₃, C₁₋₂, C₁), unsubstituted —(C₃₋₄ alkenyl) (e.g., C₃), unsubstituted —(C₃₋₄ alkynyl) (e.g., C₃), unsubstituted —(C₁₋₄ haloalkyl) (e.g., C₁₋₃, C₁₋₂, C₁), —(C₁₋₂ alkylene)C(═O)O(C₁₋₄ alkyl) (e.g., C₁₋₃, C₁₋₂, C₁), —(C₁₋₂ alkylene)C(═O)N(C₁₋₄ alkyl)₂ (e.g., C₁₋₃, C₁₋₂, C₁), —(C₁₋₂ alkylene)ₚheteroaryl optionally substituted with 1-2 (e.g., 1) R¹³, and —(C₁₋₂ alkylene)ₚaryl optionally substituted with 1-2 (e.g., 1) R¹⁴; wherein each —(C₁₋₄ alkylene) is, independently, optionally substituted with one or more substituents as defined herein.

In some embodiments of Formulas I, II, III, IV, V, and VI, R⁷ is selected from the group consisting of H, unsubstituted —(C₁₋₂ alkyl) (e.g., Me), unsubstituted —(C₃ alkenyl), unsubstituted —(C₃ alkynyl), unsubstituted —(C₁₋₂ haloalkyl) (e.g., C₁), —(CH₂)C(═O)O(C₁₋₂ alkyl) (e.g., Me), —(CH₂)C(═O)N(C₁₋₂ alkyl)₂ (e.g., Me), —(CH₂)ₚheteroaryl optionally substituted with 1 R¹³, and —(CH₂)ₚaryl optionally substituted with 1 R¹⁴.

In some embodiments of Formulas I, II, III, IV, V, and VI, R⁷ is selected from the group consisting of H, Me, —CF₃, —(CH₂)C(═O)OMe, —(CH₂)C(═O)NMe₂, -heteroaryl optionally substituted with 1 R¹³, —(CH₂)heteroaryl optionally substituted with 1 R¹³, -aryl optionally substituted with 1 R¹⁴, and —(CH₂)aryl optionally substituted with 1 R¹⁴.

In some embodiments of Formulas I, II, III, IV, V, and VI, R⁷ is selected from the group consisting of H, Me, Et, nPr, and iPr.

In some embodiments of Formulas I, II, III, IV, V, and VI, R⁷ is selected from the group consisting of H and Me.

In some embodiments of Formulas I, II, III, IV, V, and VI, R⁷ is selected from the group consisting of

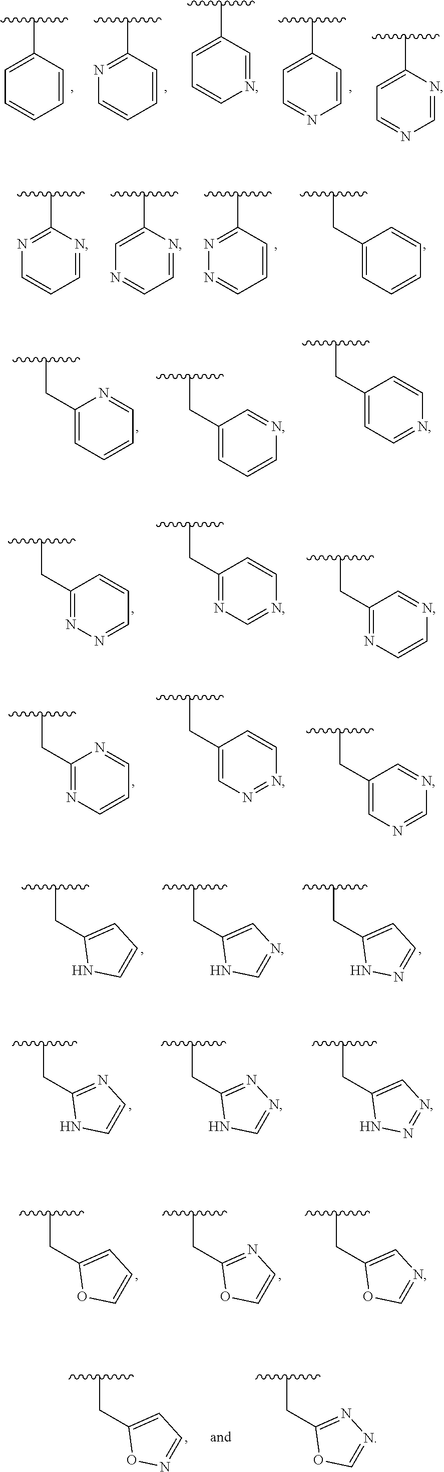

In some embodiments of Formulas I, II, III, IV, V, and VI, R⁷ is selected from the group consisting of

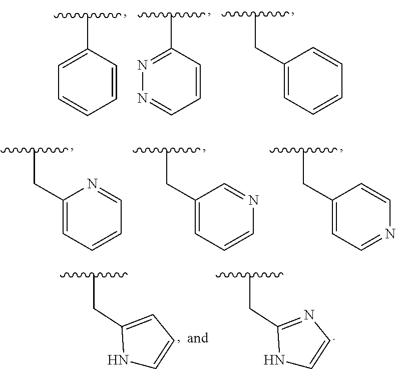

In some embodiments of Formulas I, II, III, IV, V, and VI, each R¹⁰ is independently selected from the group consisting of H, unsubstituted —(C₁₋₉ alkyl) (e.g., C₁₋₈, C₁₋₇, C₁₋₆, C₁₋₅, C₁₋₄, C₁₋₃, C₁₋₂, C₁), unsubstituted —(C₃₋₉ alkenyl) (e.g., C₃₋₈, C₃₋₇, C₃₋₆, C₃₋₅, C₃₋₄, C₃), unsubstituted —(C₃₋₉ alkynyl) (e.g., C₃₋₈, C₃₋₇, C₃₋₆, C₃₋₅, C₃₋₄, C₃), and unsubstituted —(C₁₋₉ haloalkyl) (e.g., C₁₋₈, C₁₋₇, C₁₋₆, C₁₋₅, C₁₋₄, C₁₋₃, C₁₋₂, C₁).

In some embodiments of Formulas I, II, III, IV, V, and VI, each R¹⁰ is independently selected from the group consisting of H, unsubstituted —(C₁₋₄ alkyl) (e.g., C₁₋₃, C₁₋₂, C₁), unsubstituted —(C₃₋₄ alkenyl) (e.g., C₃), unsubstituted —(C₃₋₄ alkynyl) (e.g., C₃), and unsubstituted —(C₁₋₄ haloalkyl) (e.g., C₁₋₃, C₁₋₂, C₁).

In some embodiments of Formulas I, II, III, IV, V, and VI, each R¹⁰ is independently selected from the group consisting of H, unsubstituted —(C₁₋₂ alkyl) (e.g., Me), unsubstituted —(C₃ alkenyl), unsubstituted —(C₃ alkynyl), and unsubstituted —(C₁₋₂ haloalkyl) (e.g., C₁).

In some embodiments of Formulas I, II, III, IV, V, and VI, each R¹⁰ is independently selected from the group consisting of H, Me, and —CF₃.

In some embodiments of Formulas I, II, III, IV, V, and VI, each R¹¹ is independently selected from the group consisting of H and unsubstituted —(C₁₋₉ alkyl) (e.g., C₁₋₈, C₁₋₇, C₁₋₆, C₁₋₅, C₁₋₄, C₁₋₃, C₁₋₂, C₁).

In some embodiments of Formulas I, II, III, IV, V, and VI, each R¹¹ is independently selected from the group consisting of H and unsubstituted —(C₁₋₄ alkyl) (e.g., C₁₋₃, C₁₋₂, C₁).

In some embodiments of Formulas I, II, III, IV, V, and VI, each R¹¹ is independently selected from the group consisting of H and unsubstituted —(C₁₋₂ alkyl) (e.g., Me).

In some embodiments of Formulas I, II, III, IV, V, and VI, each R¹¹ is independently selected from the group consisting of H and Me.

In some embodiments of Formulas I, II, III, IV, V, and VI, each R¹² is independently selected from the group consisting of H and substituted or unsubstituted —(C₁₋₉ alkyl) (e.g., C₁₋₈, C₁₋₇, C₁₋₆, C₁₋₅, C₁₋₄, C₁₋₃, C₁₋₂, C₁).

In some embodiments of Formulas I, II, III, IV, V, and VI, each R¹² is independently selected from the group consisting of H and substituted or unsubstituted —(C₁₋₄ alkyl) (e.g., C₁₋₃, C₁₋₂, C₁).

In some embodiments of Formulas I, II, III, IV, V, and VI, each R¹² is independently selected from the group consisting of H and substituted or unsubstituted —(C₁₋₂ alkyl) (e.g., Me).

In some embodiments of Formulas I, II, III, IV, V, and VI, each R¹² is independently selected from the group consisting of H and Me.

In some embodiments of Formulas I, II, III, IV, V, and VI, each R¹³ is independently selected from the group consisting of halide (e.g., F, Cl, Br, I), unsubstituted —(C₁₋₉ alkyl) (e.g., C₁₋₈, C₁₋₇, C₁₋₆, C₁₋₅, C₁₋₄, C₁₋₃, C₁₋₂, C₁), unsubstituted —(C₁₋₉ haloalkyl) (e.g., C₁₋₈, C₁₋₇, C₁₋₆, C₁₋₅, C₁₋₄, C₁₋₃, C₁₋₂, C₁), and —OR¹⁰.

In some embodiments of Formulas I, II, III, IV, V, and VI, each R¹³ is selected from the group consisting of H, F, Cl, Br, unsubstituted —(C₁₋₄ alkyl) (e.g., C₁₋₃, C₁₋₂, C₁), unsubstituted —(C₁₋₄ haloalkyl) (e.g., C₁₋₃, C₁₋₂, C₁), and —O(C₁₋₄ alkyl) (e.g., C₁₋₃, C₁₋₂, C₁).

In some embodiments of Formulas I, II, III, IV, V, and VI, each R¹³ is selected from the group consisting of H, F, Cl, unsubstituted —(C₁₋₂ alkyl) (e.g., Me), unsubstituted —(C₁₋₂ haloalkyl) (e.g., C₁), —OH, and —O(C₁₋₂ alkyl) (e.g., Me).

In some embodiments of Formulas I, II, III, IV, V, and VI, each R¹³ is selected from the group consisting of H, F, Cl, Me, —CF₃, —OH, and —OMe.

In some embodiments of Formulas I, II, III, IV, V, and VI, each R¹⁴ is independently selected from the group consisting of halide (e.g., F, Cl, Br, I), unsubstituted —(C₁₋₉ alkyl) (e.g., C₁₋₈, C₁₋₇, C₁₋₆, C₁₋₅, C₁₋₄, C₁₋₃, C₁₋₂, C₁), unsubstituted —(C₁₋₉ haloalkyl) (e.g., C₁₋₈, C₁₋₇, C₁₋₆, C₁₋₅, C₁₋₄, C₁₋₃, C₁₋₂, C₁), and —OR¹⁰.

In some embodiments of Formulas I, II, III, IV, V, and VI, each R¹⁴ is selected from the group consisting of H, F, Cl, Br, unsubstituted —(C₁₋₄ alkyl) (e.g., C₁₋₃, C₁₋₂, C₁), unsubstituted —(C₁₋₄ haloalkyl) (e.g., C₁₋₃, C₁₋₂, C₁), and —O(C₁₋₄ alkyl) (e.g., C₁₋₃, C₁₋₂, C₁).

In some embodiments of Formulas I, II, III, IV, V, and VI, each R¹⁴ is selected from the group consisting of H, F, Cl, unsubstituted —(C₁₋₂ alkyl) (e.g., Me), unsubstituted —(C₁₋₂ haloalkyl) (e.g., C₁), —OH, and —O(C₁₋₂ alkyl) (e.g., Me).

In some embodiments of Formulas I, II, III, IV, V, and VI, each R¹⁴ is selected from the group consisting of H, F, Cl, Me, —CF₃, —OH, and —OMe.

In some embodiments of Formulas I, II, III, IV, V, and VI, each R¹⁵ is independently selected from the group consisting of H, halide (e.g., F, Cl, Br, I), unsubstituted —(C₁₋₆ alkyl) (e.g., C₁₋₅, C₁₋₄, C₁₋₃, C₁₋₂, C₁), unsubstituted —(C₂₋₆ alkenyl) (e.g., C₂₋₈, C₂₋₄, C₂₋₃, C₂), unsubstituted —(C₂₋₆ alkynyl) (e.g., C₂₋₅, C₂₋₄, C₂₋₃, C₂), and unsubstituted —(C₁₋₆ haloalkyl) (e.g., C₁₋₅, C₁₋₄, C₁₋₃, C₁₋₂, C₁).

In some embodiments of Formulas I, II, III, IV, V, and VI, each R¹⁵ is independently selected from the group consisting of H, F, Cl, Br, unsubstituted —(C₁₋₂ alkyl) (e.g., Me), unsubstituted —(C₂ alkenyl), unsubstituted —(C₂ alkynyl), and unsubstituted —(C₁₋₂ haloalkyl) (e.g., C₁).

In some embodiments of Formulas I, II, III, IV, V, and VI, each R¹⁵ is independently selected from the group consisting of H, F, Cl, Me, and —CF₃.

In some embodiments of Formulas I, II, III, IV, and V, each R¹⁶ is independently selected from the group consisting of H, halide (e.g., F, Cl, Br, I), unsubstituted —(C₁₋₆ alkyl) (e.g., C₁₋₈, C₁₋₄, C₁₋₃, C₁₋₂, C₁), unsubstituted —(C₂₋₆ alkenyl) (e.g., C₂₋₅, C₂₋₄, C₂₋₃, C₂), unsubstituted —(C₂₋₆ alkynyl) (e.g., C₂₋₈, C₂₋₄, C₂₋₃, C₂), and unsubstituted —(C₁₋₆ haloalkyl) (e.g., C₁₋₈, C₁₋₄, C₁₋₃, C₁₋₂, C₁).

In some embodiments of Formulas I, II, III, IV, and V, each R¹⁶ is independently selected from the group consisting of H, F, Cl, Br, unsubstituted —(C₁₋₂ alkyl) (e.g., Me), unsubstituted —(C₂ alkenyl), unsubstituted —(C₂ alkynyl), and unsubstituted —(C₁₋₂ haloalkyl) (e.g., C₁).

In some embodiments of Formulas I, II, III, IV, and V, each R¹⁶ is independently selected from the group consisting of H, F, Cl, Me, and —CF₃.

In some embodiments of Formulas II, III, and IV, each R¹⁷ is selected from the group consisting of H, halide (e.g., F, Cl, Br, I), unsubstituted —(C₁₋₉ alkyl) (e.g., C₁₋₈, C₁₋₇, C₁₋₆, C₁₋₈, C₁₋₄, C₁₋₃, C₁₋₂, C₁), unsubstituted —(C₂₋₉ alkenyl) (e.g., C₂₋₈, C₂₋₇, C₂₋₆, C₂₋₅, C₂₋₄, C₂₋₃, C₂), unsubstituted —(C₂₋₉ alkynyl) (e.g., C₂₋₈, C₂₋₇, C₂₋₆, C₂₋₅, C₂₋₄, C₂₋₃, C₂), unsubstituted —(C₁₋₉ haloalkyl) (e.g., C₁₋₈, C₁₋₇, C₁₋₆, C₁₋₈, C₁₋₄, C₁₋₃, C₁₋₂, C₁), and —CN.

In some embodiments of Formulas II, III, and IV, each R¹⁷ is selected from the group consisting of H, F, Cl, Br, unsubstituted —(C₁₋₄ alkyl) (e.g., C₁₋₃, C₁₋₂, C₁), unsubstituted —(C₂₋₄ alkenyl) (e.g., C₂₋₃, C₂), unsubstituted —(C₂₋₄ alkynyl) (e.g., C₂₋₃, C₂), unsubstituted —(C₁₋₄ haloalkyl) (e.g., C₁₋₃, C₁₋₂, C₁), and —CN.

In some embodiments of Formulas II, III, and IV, each R¹⁷ is selected from the group consisting of H, F, Cl, unsubstituted —(C₁₋₂ alkyl) (e.g., Me), unsubstituted —(C₂ alkenyl), unsubstituted —(C₂ alkynyl), unsubstituted —(C₁₋₂ haloalkyl) (e.g., C₁), and —CN.

In some embodiments of Formulas II, III, and IV, each R¹⁷ is selected from the group consisting of H, F, Cl, Me, —CF₃, and —CN.

In some embodiments of Formulas III and IV, R³⁰ is selected from the group consisting of H and unsubstituted —(C₁₋₉ alkyl) (e.g., C₁₋₈, C₁₋₇, C₁₋₆, C₁₋₅, C₁₋₄, C₁₋₃, C₁₋₂, C₁).

In some embodiments of Formulas III and IV, R³⁰ is independently selected from the group consisting of H and unsubstituted —(C₁₋₄ alkyl) (e.g., C₁₋₃, C₁₋₂, C₁).

In some embodiments of Formulas III and IV, R³⁰ is independently selected from the group consisting of H and unsubstituted —(C₁₋₂ alkyl) (e.g., Me).

In some embodiments of Formulas III and IV, R³⁰ is independently selected from the group consisting of H and Me.

In some embodiments of Formulas III and IV, R³¹ is selected from the group consisting of

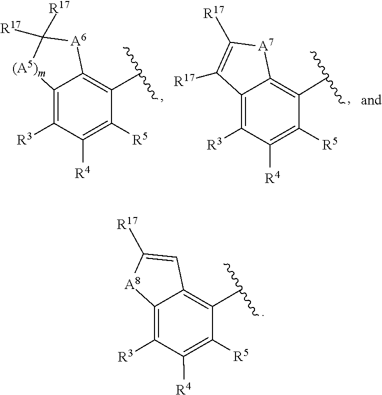

In some embodiments of Formulas III and IV, R³¹ is

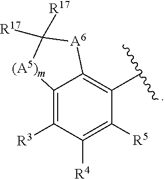

In some embodiments of Formulas III and IV, R³¹ is selected from the group consisting of

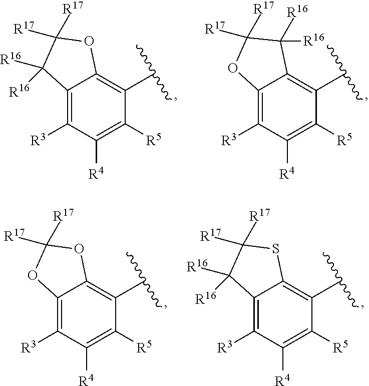

In some embodiments of Formulas III and IV, R³¹ is selected from the group consisting of

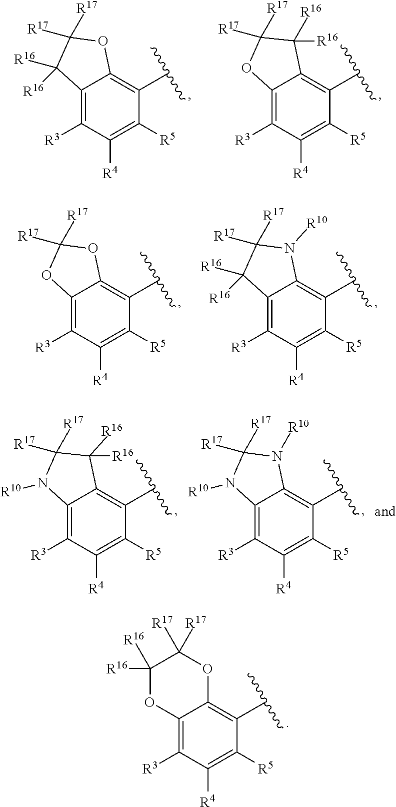

In some embodiments of Formulas III and IV, R³¹ is selected from the group consisting of

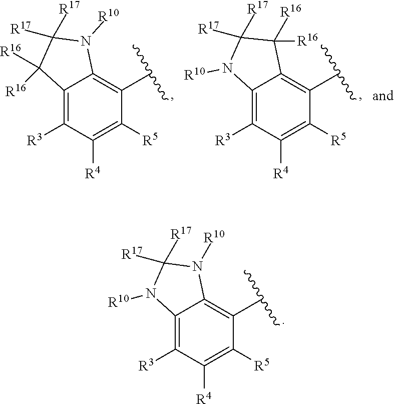

In some embodiments of Formulas III and IV, R³¹ is selected from the group consisting of

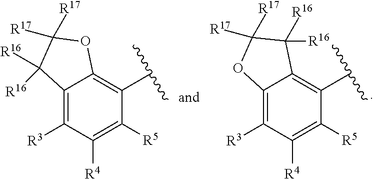

In some embodiments of Formulas III and IV, R³¹ is

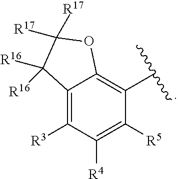

In some embodiments of Formulas III and IV, R³¹ is

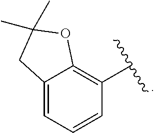 [SMILES: *c1cccc2c1OC(C)(C)C2]

In some embodiments of Formulas III and IV, R³¹ is selected from the group consisting of

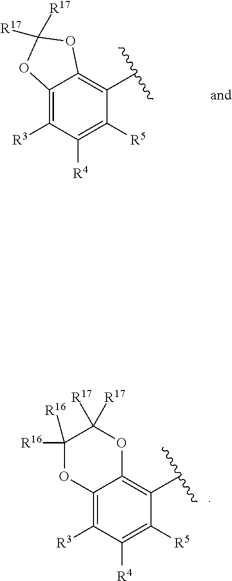

In some embodiments of Formulas III and IV, R³¹ is selected from the group consisting of

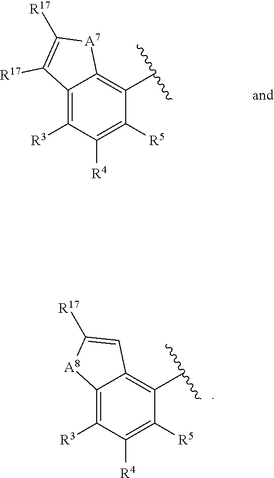

In some embodiments of Formulas III and IV, R³¹ is selected from the group consisting of

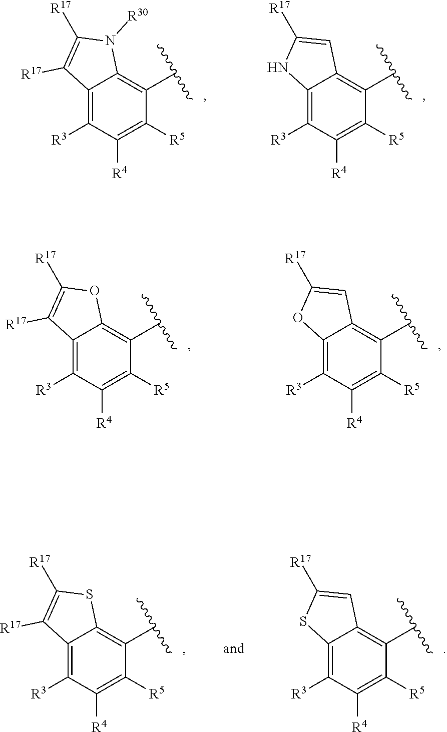

In some embodiments of Formulas I, II, III, IV, V, and VI, A¹ is selected from the group consisting of —C(R¹⁵)₂—, O, and S.

In some embodiments of Formulas I, II, III, IV, V, and VI, A¹ is selected from the group consisting of —CH₂—, O, and S.

In some embodiments of Formulas I, II, III, IV, V, and VI, A¹ is selected from the group consisting of —CH₂— and O.

In some embodiments of Formulas I, II, III, IV, V, and VI, A¹ is O.

In some embodiments of Formulas I, II, III, IV, V, and VI, A¹ is —CH₂—.

In some embodiments of Formulas I, II, IV, V, and VI, A² is selected from the group consisting of O and S.

In some embodiments of Formulas I, II, IV, V, and VI, A² is O.

In some embodiments of Formulas I, II, IV, V, and VI, A² is S.

In some embodiments of Formulas I and V, A³ is selected from the group consisting of —C(R¹⁶)₂—, O and S.

In some embodiments of Formulas I and V, A³ is selected from the group consisting of —CH₂—, O and S.

In some embodiments of Formulas I and V, A³ is selected from the group consisting of —CH₂— and O.

In some embodiments of Formulas I and V, A³ is O.

In some embodiments of Formulas I and V, A³ is —C(R¹⁶)₂—.

In some embodiments of Formulas I and V, A³ is —CH(R¹⁶)—.

In some embodiments of Formulas I and V, A³ is —CH₂—.

In some embodiments of Formulas I and V, each A⁴ is independently selected from the group consisting of —C(R¹⁶)₂—, O and S.

In some embodiments of Formulas I and V, each A⁴ is independently selected from the group consisting of —CH₂—, O and S.

In some embodiments of Formulas I and V, each A⁴ is independently selected from the group consisting of —CH₂— and O.

In some embodiments of Formulas I and V, A⁴ is O.

In some embodiments of Formulas I and V, A⁴ is —C(R¹⁶)₂—.

In some embodiments of Formulas I and V, A⁴ is —CH(R¹⁶)—.

In some embodiments of Formulas I and V, A⁴ is —CH₂—.

In some embodiments of Formulas I and V, there is the proviso that no more than two of A³ and/or A⁴ are O or S and that two O or S are never adjacent.

In some embodiments of Formulas II, III, and IV, each A⁵ is independently selected from the group consisting of —C(R¹⁶)₂—, —N(R¹⁰)—, O, and S.

In some embodiments of Formulas II, III, and IV, each A⁵ is independently selected from the group consisting of —CH₂—, —NH—, O, and S.

In some embodiments of Formulas II, III, and IV, each A⁵ is independently selected from the group consisting of —CH₂— and O.

In some embodiments of Formulas II, III, and IV, each A⁵ is independently selected from the group consisting of —CH₂— and —NH—.

In some embodiments of Formulas II, III, and IV, A⁵ is O.

In some embodiments of Formulas II, III, and IV, A⁵ is S.

In some embodiments of Formulas II, III, and IV, each A⁵ is —CH₂—.

In some embodiments of Formulas II, III, and IV, A⁵ is —NH—.

In some embodiments of Formulas II, III, and IV, there is the proviso that two A⁵ are not both N, O or S.

In some embodiments of Formulas II, III, and IV, A⁶ is selected from the group consisting of —C(R¹⁶)₂—, —N(R¹⁰)—, O, and S.

In some embodiments of Formulas II, III, and IV, A⁶ is selected from the group consisting of —CH₂—, —NH—, O, and S.

In some embodiments of Formulas II, III, and IV, A⁶ is selected from the group consisting of —CH₂— and O.

In some embodiments of Formulas II, III, and IV, A⁶ is selected from the group consisting of —CH₂— and —NH—.

In some embodiments of Formulas II, III, and IV, A⁶ is O.

In some embodiments of Formulas II, III, and IV, A⁶ is S.

In some embodiments of Formulas II, III, and IV, A⁶ is —CH₂—.

In some embodiments of Formulas II, III, and IV, A⁶ is —NH—.

In some embodiments of Formulas II, III, and IV, there is the proviso that A⁵ and A⁶ cannot both be C(R¹⁶)₂.

In some embodiments of Formulas III and IV, A⁷ and A⁸ are independently selected from the group consisting of —N(R³⁰)—, O and S.

In some embodiments of Formulas III and IV, A⁷ and A⁸ are independently selected from the group consisting of —NH—, O and S.

In some embodiments of Formulas III and IV, A⁷ and A⁸ are —NH—.

In some embodiments of Formulas III and IV, A⁷ and A⁸ are O.

In some embodiments of Formulas III and IV, A⁷ and A⁸ are S.

In some embodiments of Formulas I, III, and V, n is 1, 2, 3, or 4.

In some embodiments of Formulas I, III, and V, n is 2, 3, or 4.

In some embodiments of Formulas I, III, and V, n is 3 or 4.

In some embodiments of Formulas I, III, and V, n is 1, 2, or 3.

In some embodiments of Formulas I, III, and V, n is 1 or 2.

In some embodiments of Formulas I, III, and V, n is 1.

In some embodiments of Formulas I, III, and V, n is 2.

In some embodiments of Formulas I, III, and V, n is 3.

In some embodiments of Formulas I, III, and V, n is 4.

In some embodiments of Formulas II, III, and IV, m is 1 or 2.

In some embodiments of Formulas II, III, and IV, m is 1.

In some embodiments of Formulas II, III, and IV, m is 2.

In some embodiments of Formulas I, II, III, IV, V, and VI, each p is 0 or 1.

In some embodiments of Formulas I, II, III, IV, V, and VI, p is 0.

In some embodiments of Formulas I, II, III, IV, V, and VI, p is 1.

Illustrative compounds of Formulas I, II, III, IV, V, and VI are shown in Table 1.

TABLE 1

| | |
|---|---|
| 1 | 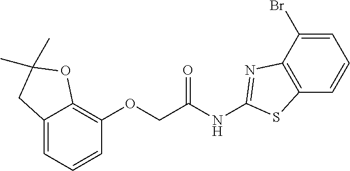 [SMILES: CC1(C)Cc2cccc(OCC(=O)Nc3nc4c(Br)cccc4s3)c2O1] |
| 2 | 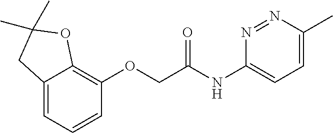 |
| 3 | 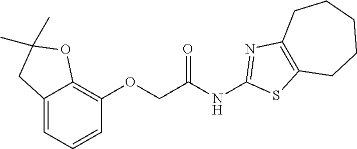 [SMILES: CC1(C)Cc2cccc(OCC(=O)Nc3nc4c(s3)CCCCC4)c2O1] |
| 4 | 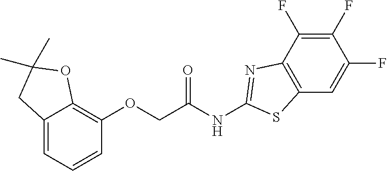 [SMILES: CC1(C)Cc2cccc(OCC(=O)Nc3nc4c(F)c(F)c(F)cc4s3)c2O1] |
| 5 | 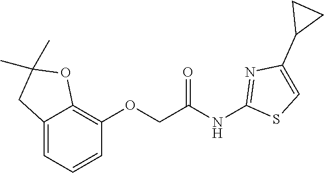 [SMILES: CC1(C)Cc2cccc(OCC(=O)Nc3nc(C4CC4)cs3)c2O1] |
| 6 | 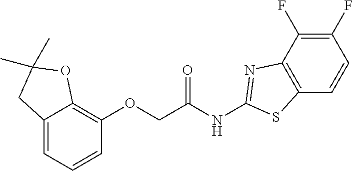 [SMILES: CC1(C)Cc2cccc(OCC(=O)Nc3nc4c(F)c(F)ccc4s3)c2O1] |
| 7 | 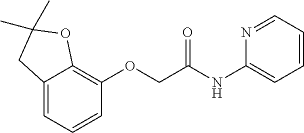 [SMILES: CC1(C)Cc2cccc(OCC(=O)Nc3ccccn3)c2O1] |
| 8 | 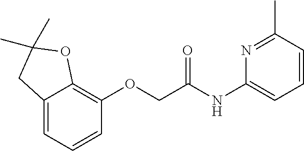 [SMILES: Cc1cccc(NC(=O)COc2cccc3c2OC(C)(C)C3)n1] |
| 9 | 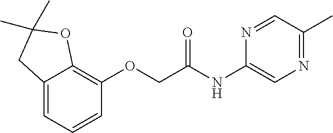 |
| 10 | 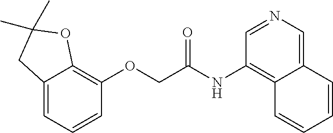 [SMILES: CC1(C)Cc2cccc(OCC(=O)Nc3cncc4ccccc34)c2O1] |
| 11 | 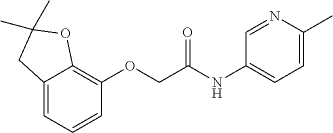 |
| 12 | 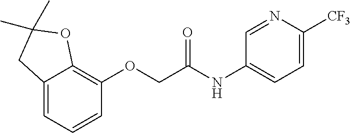 [SMILES: CC1(C)Cc2cccc(OCC(=O)Nc3ccc(C(F)(F)F)nc3)c2O1] |
| 13 | 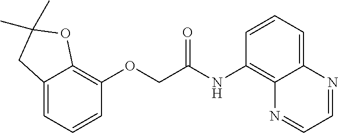 [SMILES: CC1(C)Cc2cccc(OCC(=O)Nc3cccc4nccnc34)c2O1] |
| 14 | 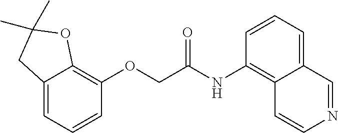 [SMILES: CC1(C)Cc2cccc(OCC(=O)Nc3cccc4cnccc34)c2O1] |
| 15 | 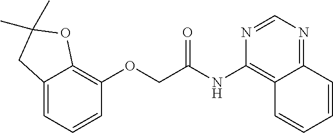 [SMILES: CC1(C)Cc2cccc(OCC(=O)Nc3ncnc4ccccc34)c2O1] |
| 16 | 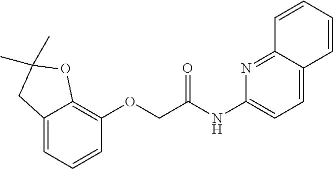 [SMILES: CC1(C)Cc2cccc(OCC(=O)Nc3ccc4ccccc4n3)c2O1] |
| 17 | 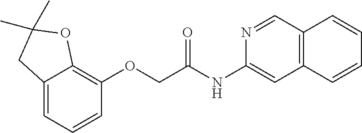 [SMILES: CC1(C)Cc2cccc(OCC(=O)Nc3cc4ccccc4cn3)c2O1] |
| 18 | 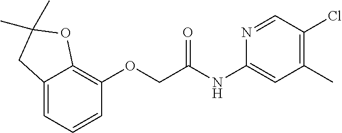 [SMILES: Cc1cc(NC(=O)COc2cccc3c2OC(C)(C)C3)ncc1Cl] |
| 19 | 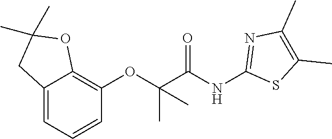 |
| 20 | 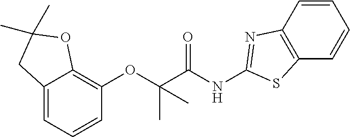 [SMILES: CC1(C)Cc2cccc(OC(C)(C)C(=O)Nc3nc4ccccc4s3)c2O1] |
| 21 | 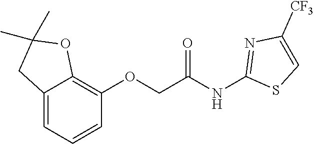 [SMILES: CC1(C)Cc2cccc(OCC(=O)Nc3nc(C(F)(F)F)cs3)c2O1] |
| 22 | 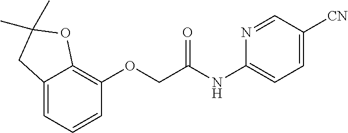 [SMILES: CC1(C)Cc2cccc(OCC(=O)Nc3ccc(C#N)cn3)c2O1] |
| 23 | 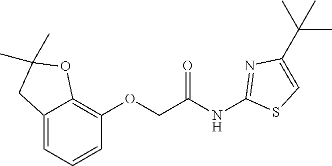 [SMILES: CC1(C)Cc2cccc(OCC(=O)Nc3nc(C(C)(C)C)cs3)c2O1] |
| 24 | 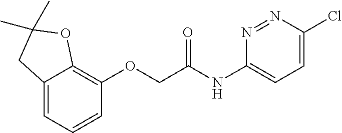 [SMILES: CC1(C)Cc2cccc(OCC(=O)Nc3ccc(Cl)nn3)c2O1] |
| 25 | 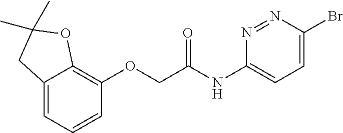 [SMILES: CC1(C)Cc2cccc(OCC(=O)Nc3ccc(Br)nn3)c2O1] |
| 26 | 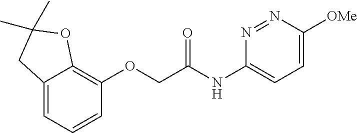 [SMILES: COc1ccc(NC(=O)COc2cccc3c2OC(C)(C)C3)nn1] |
| 27 | 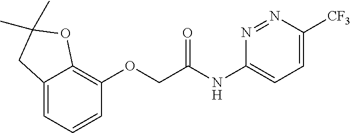 [SMILES: CC1(C)Cc2cccc(OCC(=O)Nc3ccc(C(F)(F)F)nn3)c2O1] |
| 28 | 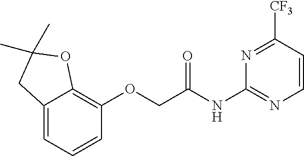 [SMILES: CC1(C)Cc2cccc(OCC(=O)Nc3nccc(C(F)(F)F)n3)c2O1] |
| 29 | 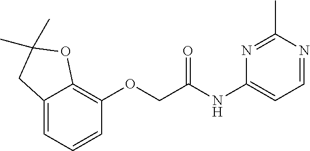 [SMILES: Cc1nccc(NC(=O)COc2cccc3c2OC(C)(C)C3)n1] |
| 30 | 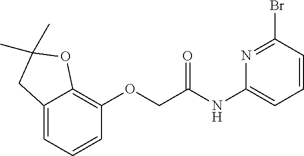 [SMILES: CC1(C)Cc2cccc(OCC(=O)Nc3cccc(Br)n3)c2O1] |
| 31 | 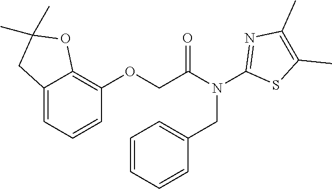 |
| 32 | 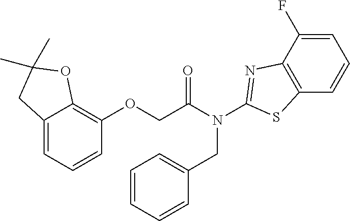 [SMILES: CC1(C)Cc2cccc(OCC(=O)N(Cc3ccccc3)c3nc4c(F)cccc4s3)c2O1] |
| 33 | 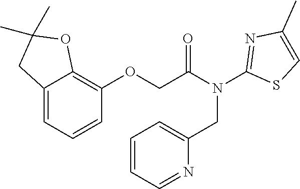 [SMILES: Cc1csc(N(Cc2ccccn2)C(=O)COc2cccc3c2OC(C)(C)C3)n1] |
| 34 | 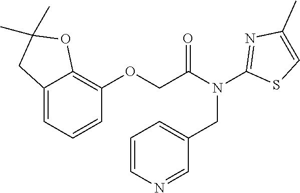 [SMILES: Cc1csc(N(Cc2cccnc2)C(=O)COc2cccc3c2OC(C)(C)C3)n1] |
| 35 | 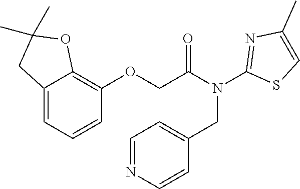 [SMILES: Cc1csc(N(Cc2ccncc2)C(=O)COc2cccc3c2OC(C)(C)C3)n1] |
| 36 | 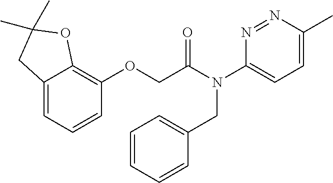 |
| 37 | 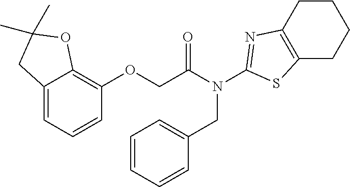 [SMILES: CC1(C)Cc2cccc(OCC(=O)N(Cc3ccccc3)c3nc4c(s3)CCCC4)c2O1] |
| 38 | 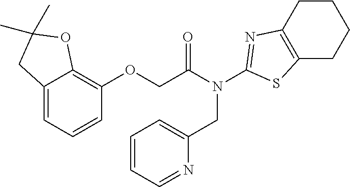 [SMILES: CC1(C)Cc2cccc(OCC(=O)N(Cc3ccccn3)c3nc4c(s3)CCCC4)c2O1] |
| 39 | 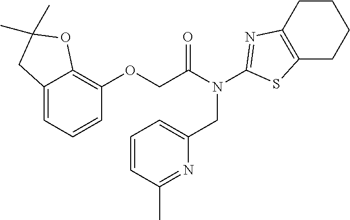 [SMILES: Cc1cccc(CN(C(=O)COc2cccc3c2OC(C)(C)C3)c2nc3c(s2)CCCC3)n1] |
| 40 | 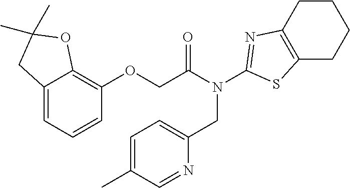 [SMILES: Cc1ccc(CN(C(=O)COc2cccc3c2OC(C)(C)C3)c2nc3c(s2)CCCC3)nc1] |
| 41 | 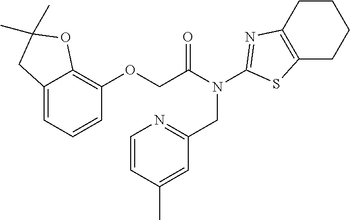 [SMILES: Cc1ccnc(CN(C(=O)COc2cccc3c2OC(C)(C)C3)c2nc3c(s2)CCCC3)c1] |
| 42 | 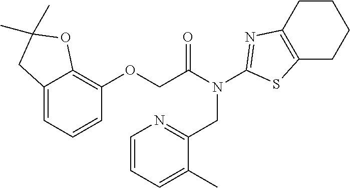 [SMILES: Cc1cccnc1CN(C(=O)COc1cccc2c1OC(C)(C)C2)c1nc2c(s1)CCCC2] |
| 43 | 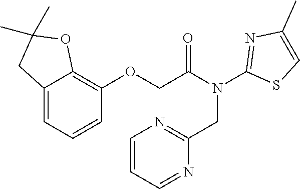 [SMILES: Cc1csc(N(Cc2ncccn2)C(=O)COc2cccc3c2OC(C)(C)C3)n1] |
| 44 | 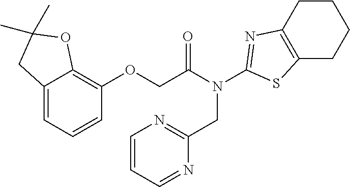 [SMILES: CC1(C)Cc2cccc(OCC(=O)N(Cc3ncccn3)c3nc4c(s3)CCCC4)c2O1] |
| 45 | 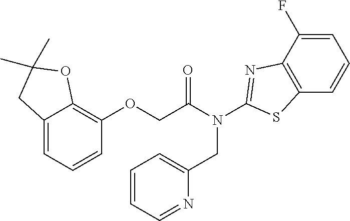 [SMILES: CC1(C)Cc2cccc(OCC(=O)N(Cc3ccccn3)c3nc4c(F)cccc4s3)c2O1] |
| 46 | 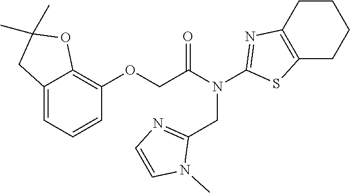 [SMILES: Cn1ccnc1CN(C(=O)COc1cccc2c1OC(C)(C)C2)c1nc2c(s1)CCCC2] |
| 47 | 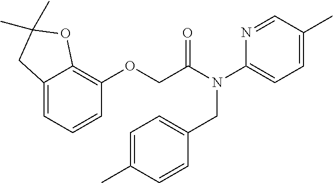 |
| 48 | 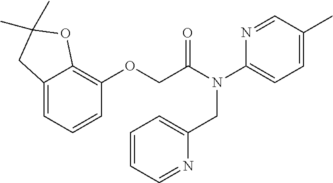 |
| 49 | 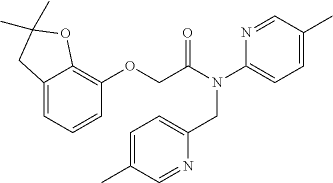 |
| 50 | 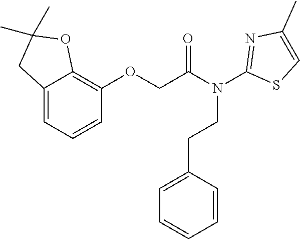 [SMILES: Cc1csc(N(CCc2ccccc2)C(=O)COc2cccc3c2OC(C)(C)C3)n1] |
| 51 | 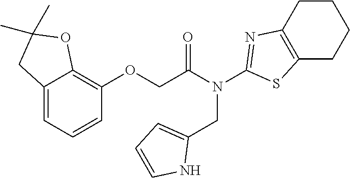 [SMILES: CC1(C)Cc2cccc(OCC(=O)N(Cc3ccc[nH]3)c3nc4c(s3)CCCC4)c2O1] |
| 52 | 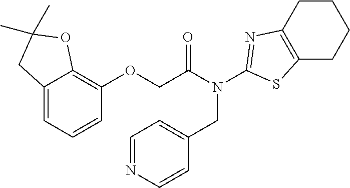 [SMILES: CC1(C)Cc2cccc(OCC(=O)N(Cc3ccncc3)c3nc4c(s3)CCCC4)c2O1] |
| 53 | 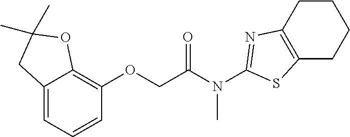 [SMILES: CN(C(=O)COc1cccc2c1OC(C)(C)C2)c1nc2c(s1)CCCC2] |
| 54 | 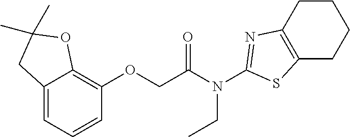 [SMILES: CCN(C(=O)COc1cccc2c1OC(C)(C)C2)c1nc2c(s1)CCCC2] |
| 55 | 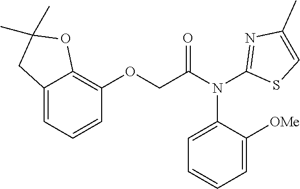 [SMILES: COc1ccccc1N(C(=O)COc1cccc2c1OC(C)(C)C2)c1nc(C)cs1] |
| 56 | 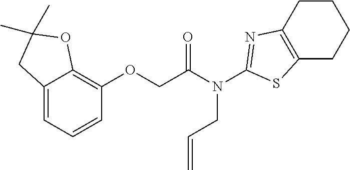 [SMILES: C=CCN(C(=O)COc1cccc2c1OC(C)(C)C2)c1nc2c(s1)CCCC2] |
| 57 | 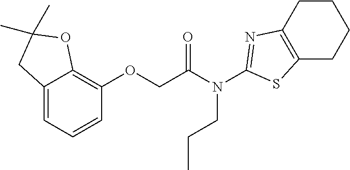 [SMILES: CCCN(C(=O)COc1cccc2c1OC(C)(C)C2)c1nc2c(s1)CCCC2] |
| 58 | 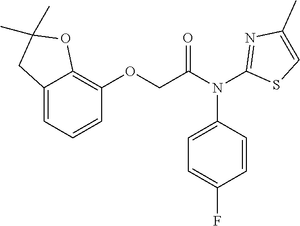 [SMILES: Cc1csc(N(C(=O)COc2cccc3c2OC(C)(C)C3)c2ccc(F)cc2)n1] |
| 59 | 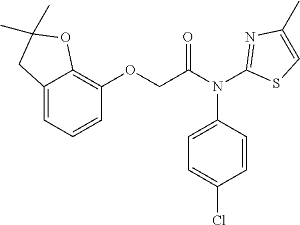 |
| 60 | 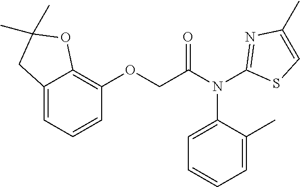 |
| 61 | 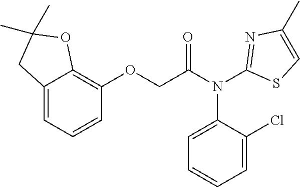 |
| 62 | 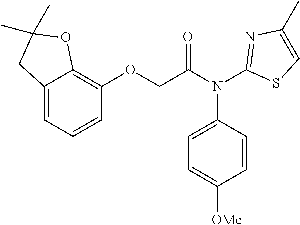 |
| 63 | 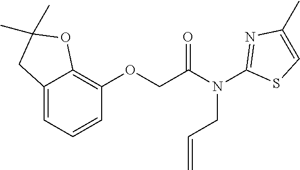 |
| 64 | 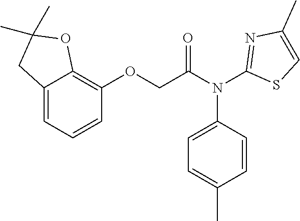 [SMILES: Cc1ccc(N(C(=O)COc2cccc3c2OC(C)(C)C3)c2nc(C)cs2)cc1] |
| 65 | 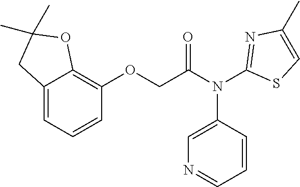 |
| 67 | 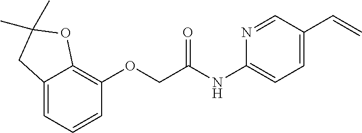 [SMILES: C=Cc1ccc(NC(=O)COc2cccc3c2OC(C)(C)C3)nc1] |
| 68 | 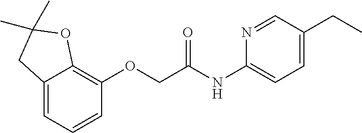 [SMILES: CCc1ccc(NC(=O)COc2cccc3c2OC(C)(C)C3)nc1] |
| 69 | 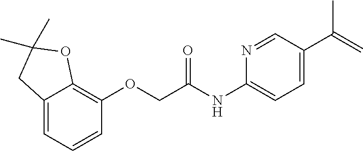 [SMILES: C=C(C)c1ccc(NC(=O)COc2cccc3c2OC(C)(C)C3)nc1] |
| 70 | 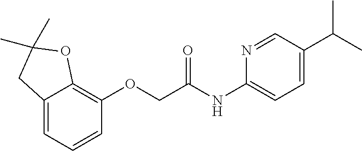 [SMILES: CC(C)c1ccc(NC(=O)COc2cccc3c2OC(C)(C)C3)nc1] |
| 71 | 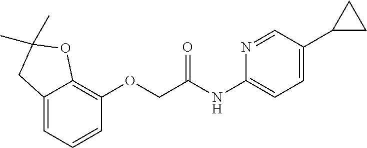 [SMILES: CC1(C)Cc2cccc(OCC(=O)Nc3ccc(C4CC4)cn3)c2O1] |
| 73 | 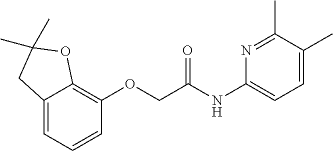 |
| 74 | 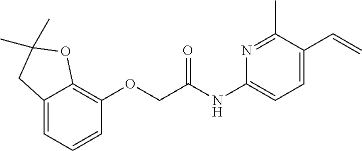 [SMILES: C=Cc1ccc(NC(=O)COc2cccc3c2OC(C)(C)C3)nc1C] |
| 75 | 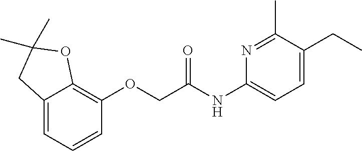 [SMILES: CCc1ccc(NC(=O)COc2cccc3c2OC(C)(C)C3)nc1C] |
| 76 | 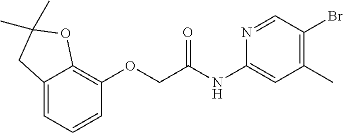 |
| 77 | 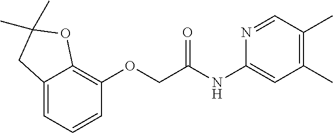 |
| 78 | 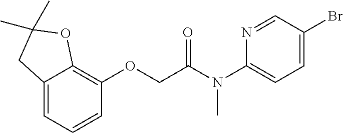 [SMILES: CN(C(=O)COc1cccc2c1OC(C)(C)C2)c1ccc(Br)cn1] |
| 79 | 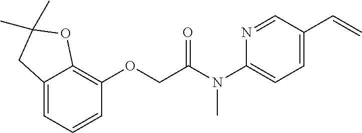 [SMILES: C=Cc1ccc(N(C)C(=O)COc2cccc3c2OC(C)(C)C3)nc1] |
| 80 | 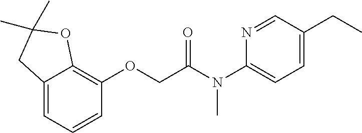 [SMILES: CCc1ccc(N(C)C(=O)COc2cccc3c2OC(C)(C)C3)nc1] |
| 81 | 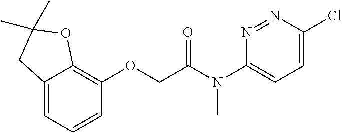 [SMILES: CN(C(=O)COc1cccc2c1OC(C)(C)C2)c1ccc(Cl)nn1] |
| 82 | 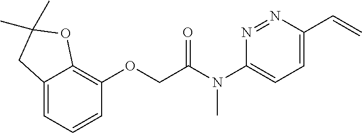 [SMILES: C=Cc1ccc(N(C)C(=O)COc2cccc3c2OC(C)(C)C3)nn1] |
| 83 | 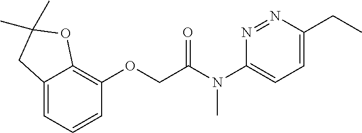 [SMILES: CCc1ccc(N(C)C(=O)COc2cccc3c2OC(C)(C)C3)nn1] |
| 84 | 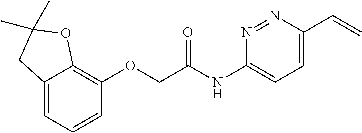 [SMILES: C=Cc1ccc(NC(=O)COc2cccc3c2OC(C)(C)C3)nn1] |
| 85 | 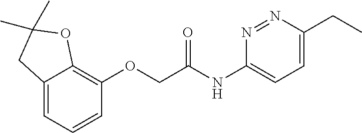 [SMILES: CCc1ccc(NC(=O)COc2cccc3c2OC(C)(C)C3)nn1] |
| 86 | 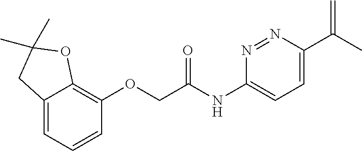 [SMILES: C=C(C)c1ccc(NC(=O)COc2cccc3c2OC(C)(C)C3)nn1] |
| 87 | 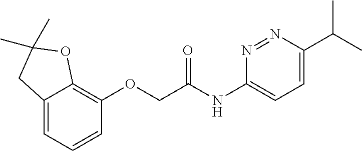 [SMILES: CC(C)c1ccc(NC(=O)COc2cccc3c2OC(C)(C)C3)nn1] |
| 88 | 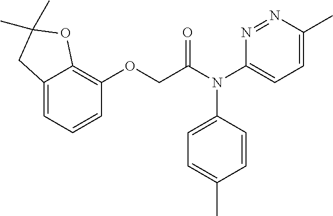 |
| 89 | 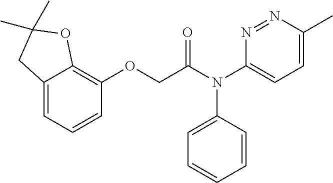 |
| 90 | 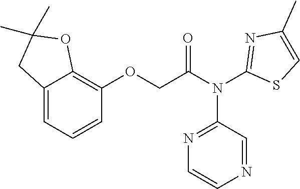 [SMILES: Cc1csc(N(C(=O)COc2cccc3c2OC(C)(C)C3)c2cnccn2)n1] |
| 91 | 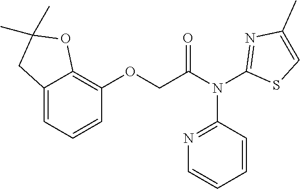 [SMILES: Cc1csc(N(C(=O)COc2cccc3c2OC(C)(C)C3)c2ccccn2)n1] |
| 92 | 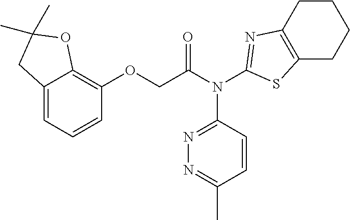 [SMILES: Cc1ccc(N(C(=O)COc2cccc3c2OC(C)(C)C3)c2nc3c(s2)CCCC3)nn1] |
| 93 | 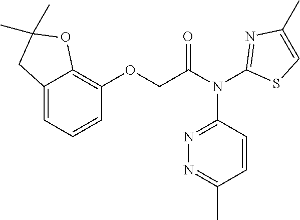 |
| 94 | 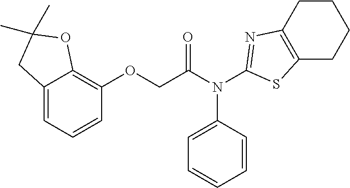 [SMILES: CC1(C)Cc2cccc(OCC(=O)N(c3ccccc3)c3nc4c(s3)CCCC4)c2O1] |
| 95 | 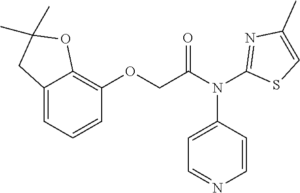 [SMILES: Cc1csc(N(C(=O)COc2cccc3c2OC(C)(C)C3)c2ccncc2)n1] |
| 96 | 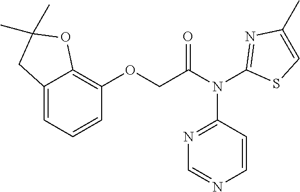 |
| 97 | 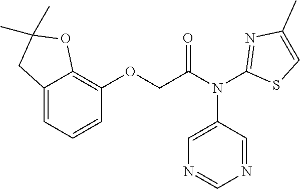 [SMILES: Cc1csc(N(C(=O)COc2cccc3c2OC(C)(C)C3)c2cncnc2)n1] |
| 98 | 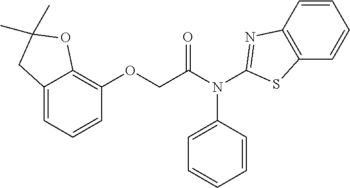 [SMILES: CC1(C)Cc2cccc(OCC(=O)N(c3ccccc3)c3nc4ccccc4s3)c2O1] |
| 99 | 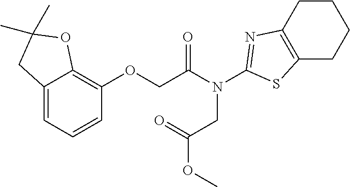 [SMILES: COC(=O)CN(C(=O)COc1cccc2c1OC(C)(C)C2)c1nc2c(s1)CCCC2] |
| 100 | 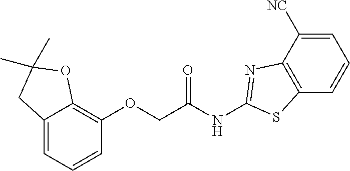 [SMILES: CC1(C)Cc2cccc(OCC(=O)Nc3nc4c(C#N)cccc4s3)c2O1] |
| 101 | 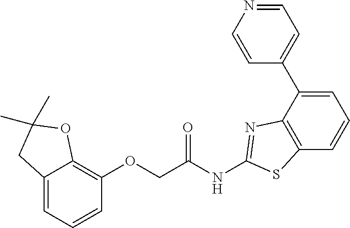 [SMILES: CC1(C)Cc2cccc(OCC(=O)Nc3nc4c(-c5ccncc5)cccc4s3)c2O1] |
| 102 | 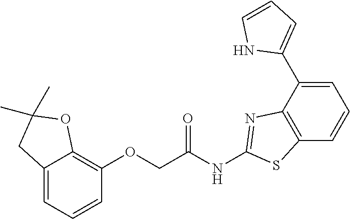 [SMILES: CC1(C)Cc2cccc(OCC(=O)Nc3nc4c(-c5ccc[nH]5)cccc4s3)c2O1] |
| 103 | 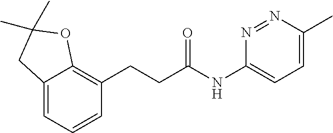 |
| 104 | 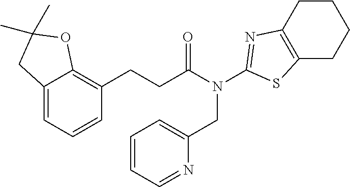 [SMILES: CC1(C)Cc2cccc(CCC(=O)N(Cc3ccccn3)c3nc4c(s3)CCCC4)c2O1] |
| 105 | 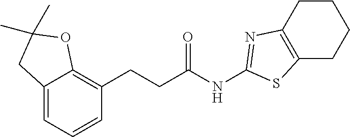 [SMILES: CC1(C)Cc2cccc(CCC(=O)Nc3nc4c(s3)CCCC4)c2O1] |
| 106 | 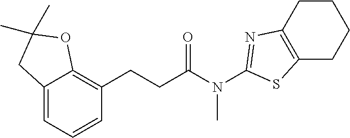 [SMILES: CN(C(=O)CCc1cccc2c1OC(C)(C)C2)c1nc2c(s1)CCCC2] |
| 107 | 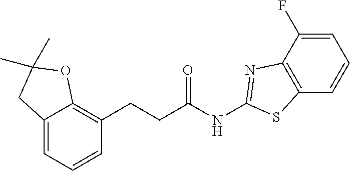 [SMILES: CC1(C)Cc2cccc(CCC(=O)Nc3nc4c(F)cccc4s3)c2O1] |
| 108 | 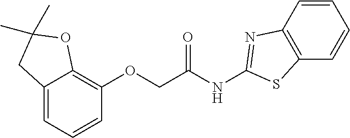 [SMILES: CC1(C)Cc2cccc(OCC(=O)Nc3nc4ccccc4s3)c2O1] |
| 109 | 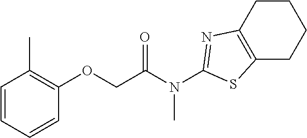 [SMILES: Cc1ccccc1OCC(=O)N(C)c1nc2c(s1)CCCC2] |
| 110 | 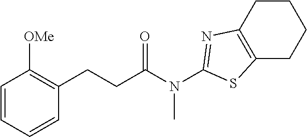 [SMILES: COc1ccccc1CCC(=O)N(C)c1nc2c(s1)CCCC2] |
| 111 | 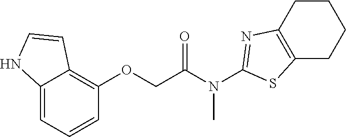 [SMILES: CN(C(=O)COc1cccc2[nH]ccc12)c1nc2c(s1)CCCC2] |
| 112 | 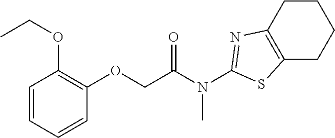 [SMILES: CCOc1ccccc1OCC(=O)N(C)c1nc2c(s1)CCCC2] |
| 113 | 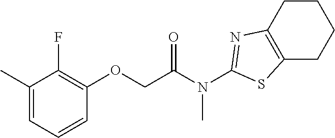 [SMILES: Cc1cccc(OCC(=O)N(C)c2nc3c(s2)CCCC3)c1F] |
| 114 | 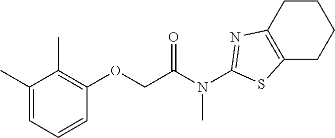 [SMILES: Cc1cccc(OCC(=O)N(C)c2nc3c(s2)CCCC3)c1C] |
| 115 | 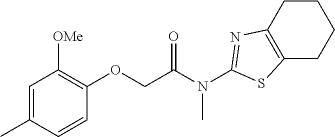 [SMILES: COc1cc(C)ccc1OCC(=O)N(C)c1nc2c(s1)CCCC2] |
| 116 | 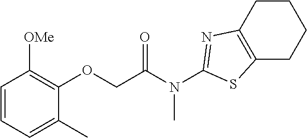 [SMILES: COc1cccc(C)c1OCC(=O)N(C)c1nc2c(s1)CCCC2] |
| 117 | 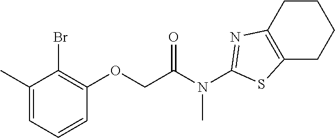 [SMILES: Cc1cccc(OCC(=O)N(C)c2nc3c(s2)CCCC3)c1Br] |
| 118 | 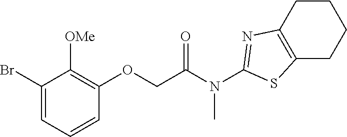 [SMILES: COc1c(Br)cccc1OCC(=O)N(C)c1nc2c(s1)CCCC2] |
| 119 | 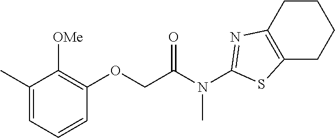 [SMILES: COc1c(C)cccc1OCC(=O)N(C)c1nc2c(s1)CCCC2] |
| 120 | 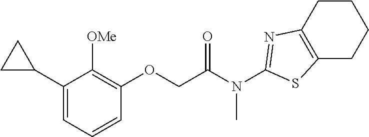 [SMILES: COc1c(OCC(=O)N(C)c2nc3c(s2)CCCC3)cccc1C1CC1] |
| 121 | 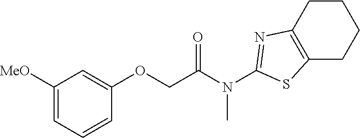 [SMILES: COc1cccc(OCC(=O)N(C)c2nc3c(s2)CCCC3)c1] |
| 122 | 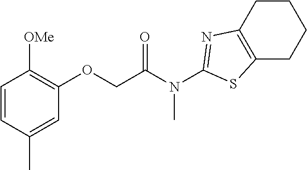 [SMILES: COc1ccc(C)cc1OCC(=O)N(C)c1nc2c(s1)CCCC2] |
| 123 | 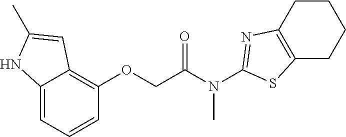 [SMILES: Cc1cc2c(OCC(=O)N(C)c3nc4c(s3)CCCC4)cccc2[nH]1] |
| 124 | 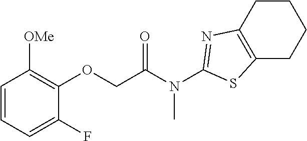 [SMILES: COc1cccc(F)c1OCC(=O)N(C)c1nc2c(s1)CCCC2] |
| 125 | 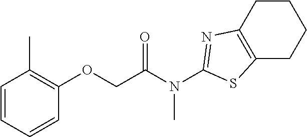 [SMILES: Cc1ccccc1OCC(=O)N(C)c1nc2c(s1)CCCC2] |
| 126 | 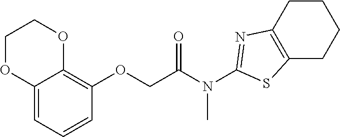 [SMILES: CN(C(=O)COc1cccc2c1OCCO2)c1nc2c(s1)CCCC2] |
| 127 | 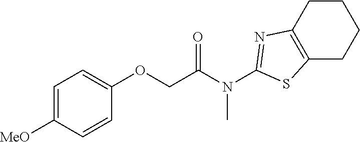 [SMILES: COc1ccc(OCC(=O)N(C)c2nc3c(s2)CCCC3)cc1] |
| 128 | 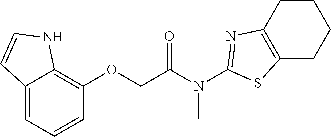 [SMILES: CN(C(=O)COc1cccc2cc[nH]c12)c1nc2c(s1)CCCC2] |
| 129 | 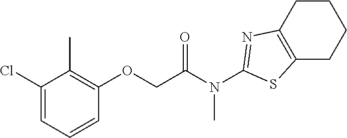 [SMILES: Cc1c(Cl)cccc1OCC(=O)N(C)c1nc2c(s1)CCCC2] |
| 130 | 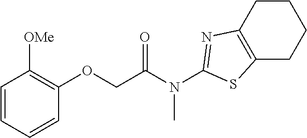 [SMILES: COc1ccccc1OCC(=O)N(C)c1nc2c(s1)CCCC2] |
| 131 | 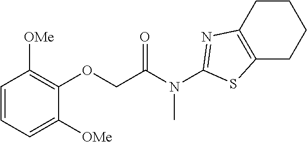 [SMILES: COc1cccc(OC)c1OCC(=O)N(C)c1nc2c(s1)CCCC2] |
| 132 | 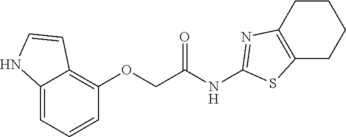 [SMILES: O=C(COc1cccc2[nH]ccc12)Nc1nc2c(s1)CCCC2] |
| 133 | 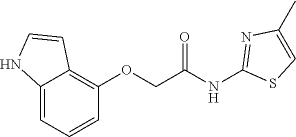 [SMILES: Cc1csc(NC(=O)COc2cccc3[nH]ccc23)n1] |
| 134 | 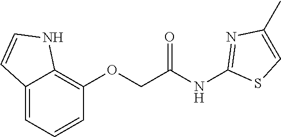 [SMILES: Cc1csc(NC(=O)COc2cccc3cc[nH]c23)n1] |
| 135 | 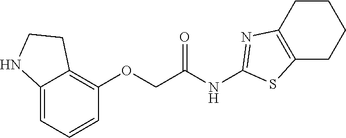 [SMILES: O=C(COc1cccc2c1CCN2)Nc1nc2c(s1)CCCC2] |
| 136 | 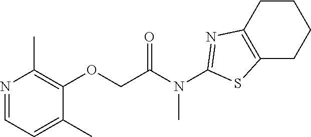 [SMILES: Cc1ccnc(C)c1OCC(=O)N(C)c1nc2c(s1)CCCC2] |
| 137 | 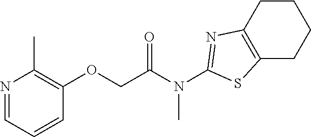 [SMILES: Cc1ncccc1OCC(=O)N(C)c1nc2c(s1)CCCC2] |
| 138 | 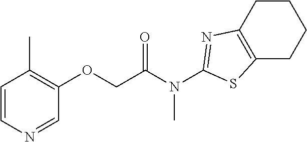 [SMILES: Cc1ccncc1OCC(=O)N(C)c1nc2c(s1)CCCC2] |
| 139 | 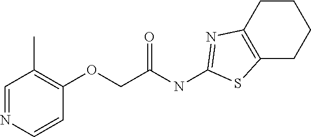 [SMILES: Cc1cnccc1OCC(=O)Nc1nc2c(s1)CCCC2] |
| 140 | 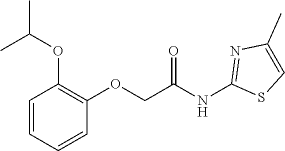 [SMILES: Cc1csc(NC(=O)COc2ccccc2OC(C)C)n1] |
| 141 | 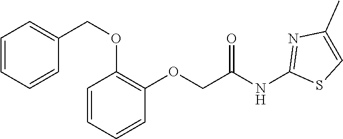 [SMILES: Cc1csc(NC(=O)COc2ccccc2OCc2ccccc2)n1] |
| 142 | 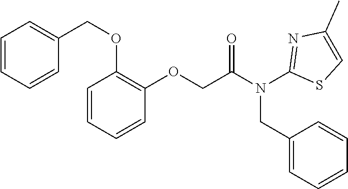 [SMILES: Cc1csc(N(Cc2ccccc2)C(=O)COc2ccccc2OCc2ccccc2)n1] |
| 144 | 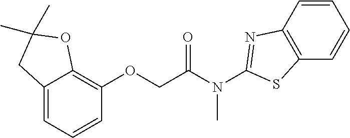 [SMILES: CN(C(=O)COc1cccc2c1OC(C)(C)C2)c1nc2ccccc2s1] |
| 145 | 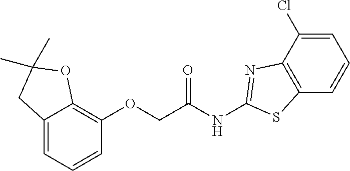 [SMILES: CC1(C)Cc2cccc(OCC(=O)Nc3nc4c(Cl)cccc4s3)c2O1] |
| 146 | 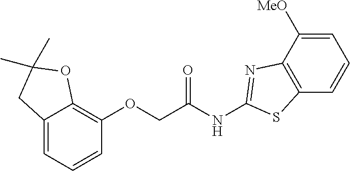 [SMILES: COc1cccc2sc(NC(=O)COc3cccc4c3OC(C)(C)C4)nc12] |
| 147 | 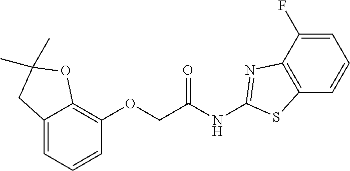 [SMILES: CC1(C)Cc2cccc(OCC(=O)Nc3nc4c(F)cccc4s3)c2O1] |
| 148 | 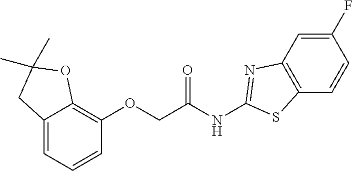 [SMILES: CC1(C)Cc2cccc(OCC(=O)Nc3nc4cc(F)ccc4s3)c2O1] |
| 149 | 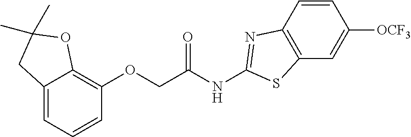 [SMILES: CC1(C)Cc2cccc(OCC(=O)Nc3nc4ccc(OC(F)(F)F)cc4s3)c2O1] |
| 150 | 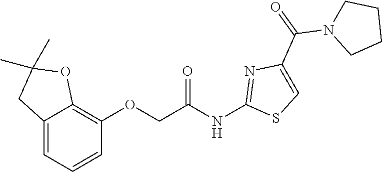 [SMILES: CC1(C)Cc2cccc(OCC(=O)Nc3nc(C(=O)N4CCCC4)cs3)c2O1] |
| 151 | 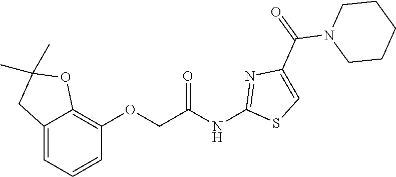 [SMILES: CC1(C)Cc2cccc(OCC(=O)Nc3nc(C(=O)N4CCCCC4)cs3)c2O1] |
| 152 | 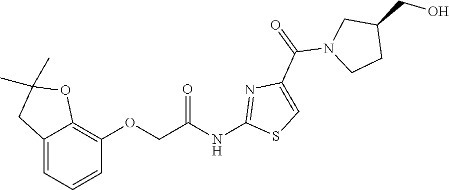 [SMILES: CC1(C)Cc2cccc(OCC(=O)Nc3nc(C(=O)N4CC[C@H](CO)C4)cs3)c2O1] |
| 153 | 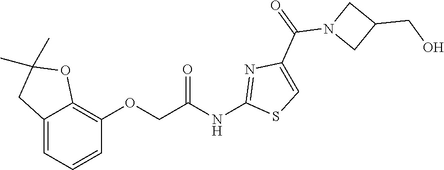 [SMILES: CC1(C)Cc2cccc(OCC(=O)Nc3nc(C(=O)N4CC(CO)C4)cs3)c2O1] |
| 154 | 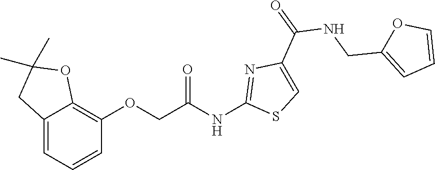 [SMILES: CC1(C)Cc2cccc(OCC(=O)Nc3nc(C(=O)NCc4ccco4)cs3)c2O1] |
| 155 | 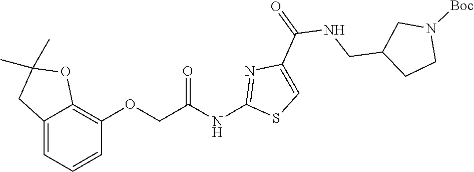 [SMILES: CC(C)(C)OC(=O)N1CCC(CNC(=O)c2csc(NC(=O)COc3cccc4c3OC(C)(C)C4)n2)C1] |
| 156 | 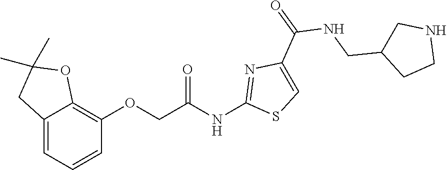 [SMILES: CC1(C)Cc2cccc(OCC(=O)Nc3nc(C(=O)NCC4CCNC4)cs3)c2O1] |
| 157 | 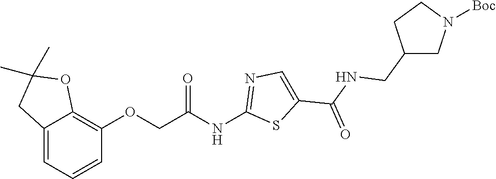 [SMILES: CC(C)(C)OC(=O)N1CCC(CNC(=O)c2cnc(NC(=O)COc3cccc4c3OC(C)(C)C4)s2)C1] |
| 158 | 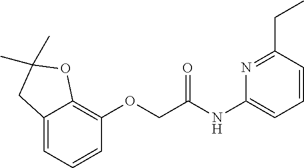 [SMILES: CCc1cccc(NC(=O)COc2cccc3c2OC(C)(C)C3)n1] |
| 159 | 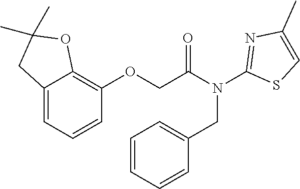 [SMILES: Cc1csc(N(Cc2ccccc2)C(=O)COc2cccc3c2OC(C)(C)C3)n1] |
| 160 | 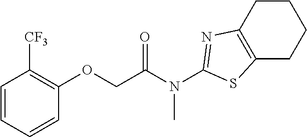 [SMILES: CN(C(=O)COc1ccccc1C(F)(F)F)c1nc2c(s1)CCCC2] |
| 161 | 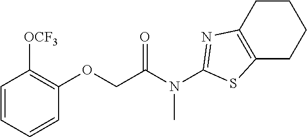 [SMILES: CN(C(=O)COc1ccccc1OC(F)(F)F)c1nc2c(s1)CCCC2] |
| 162 | 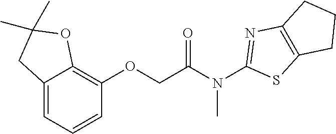 [SMILES: CN(C(=O)COc1cccc2c1OC(C)(C)C2)c1nc2c(s1)CCC2] |
| 163 | 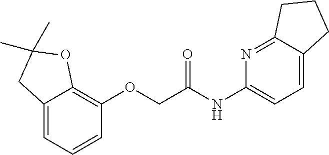 [SMILES: CC1(C)Cc2cccc(OCC(=O)Nc3ccc4c(n3)CCC4)c2O1] |
| 164 | 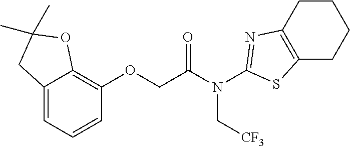 [SMILES: CC1(C)Cc2cccc(OCC(=O)N(CC(F)(F)F)c3nc4c(s3)CCCC4)c2O1] |
| 165 | 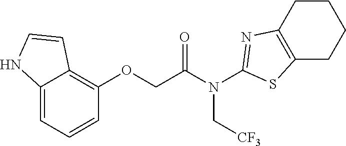 [SMILES: O=C(COc1cccc2[nH]ccc12)N(CC(F)(F)F)c1nc2c(s1)CCCC2] |
| 166 | 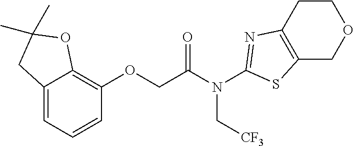 [SMILES: CC1(C)Cc2cccc(OCC(=O)N(CC(F)(F)F)c3nc4c(s3)COCC4)c2O1] |
| 167 | 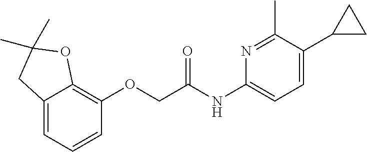 [SMILES: Cc1nc(NC(=O)COc2cccc3c2OC(C)(C)C3)ccc1C1CC1] |
| 168 | 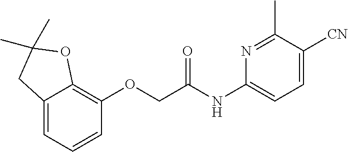 [SMILES: Cc1nc(NC(=O)COc2cccc3c2OC(C)(C)C3)ccc1C#N] |
| 169 | 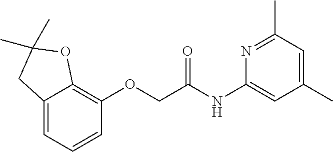 |
| 170 | 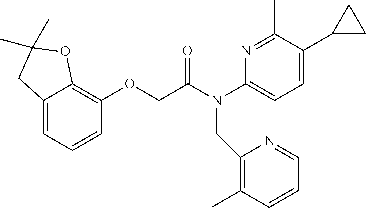 [SMILES: Cc1cccnc1CN(C(=O)COc1cccc2c1OC(C)(C)C2)c1ccc(C2CC2)c(C)n1] |
| 171 | 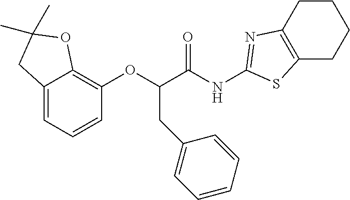 [SMILES: CC1(C)Cc2cccc(OC(Cc3ccccc3)C(=O)Nc3nc4c(s3)CCCC4)c2O1] |
| 172 | 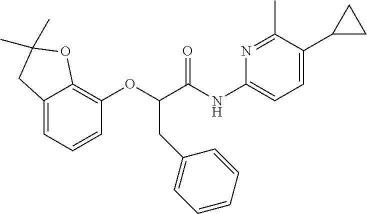 [SMILES: Cc1nc(NC(=O)C(Cc2ccccc2)Oc2cccc3c2OC(C)(C)C3)ccc1C1CC1] |
| 173 | 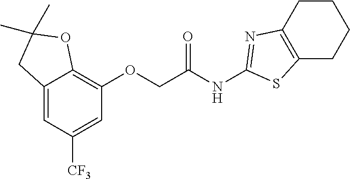 [SMILES: CC1(C)Cc2cc(C(F)(F)F)cc(OCC(=O)Nc3nc4c(s3)CCCC4)c2O1] |
| 174 | 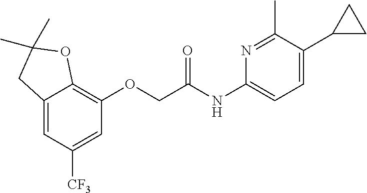 [SMILES: Cc1nc(NC(=O)COc2cc(C(F)(F)F)cc3c2OC(C)(C)C3)ccc1C1CC1] |

Additional illustrative compounds are shown in Table 2.

TABLE 2

| | |
|---|---|
| 66 | 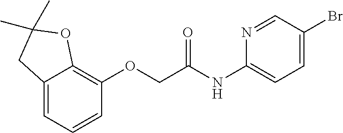 [SMILES: CC1(C)Cc2cccc(OCC(=O)Nc3ccc(Br)cn3)c2O1] |
| 72 | 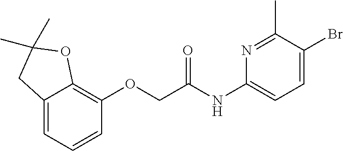 [SMILES: Cc1nc(NC(=O)COc2cccc3c2OC(C)(C)C3)ccc1Br] |
| 143 | 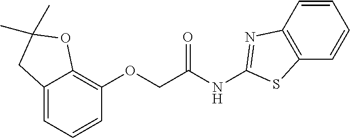 [SMILES: CC1(C)Cc2cccc(OCC(=O)Nc3nc4ccccc4s3)c2O1] |
| 175 | 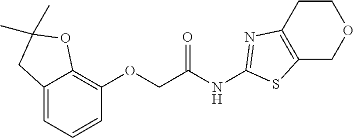 [SMILES: CC1(C)Cc2cccc(OCC(=O)Nc3nc4c(s3)COCC4)c2O1] |
| 176 | 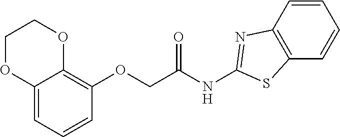 [SMILES: O=C(COc1cccc2c1OCCO2)Nc1nc2ccccc2s1] |
| 177 | 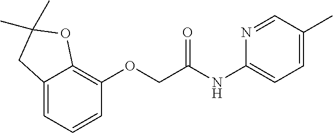 |
| 178 | 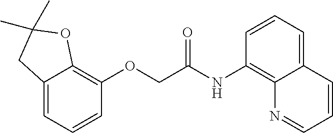 [SMILES: CC1(C)Cc2cccc(OCC(=O)Nc3cccc4cccnc34)c2O1] |
| 179 | 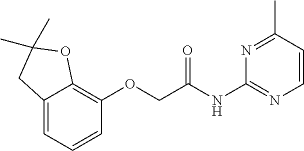 [SMILES: Cc1ccnc(NC(=O)COc2cccc3c2OC(C)(C)C3)n1] |
| 180 | 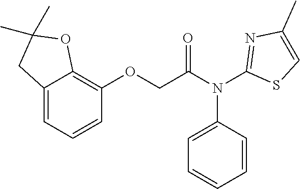 [SMILES: Cc1csc(N(C(=O)COc2cccc3c2OC(C)(C)C3)c2ccccc2)n1] |
| 181 | 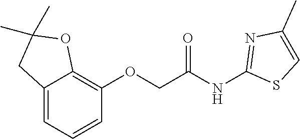 [SMILES: Cc1csc(NC(=O)COc2cccc3c2OC(C)(C)C3)n1] |
| 182 | 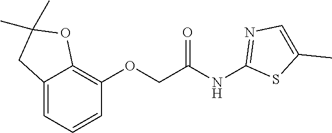 |
| 183 | 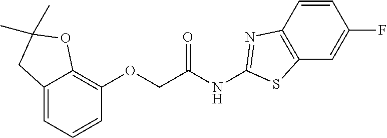 [SMILES: CC1(C)Cc2cccc(OCC(=O)Nc3nc4ccc(F)cc4s3)c2O1] |
| 184 | 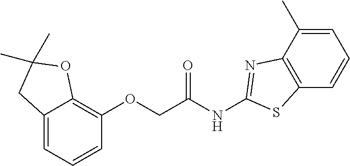 [SMILES: Cc1cccc2sc(NC(=O)COc3cccc4c3OC(C)(C)C4)nc12] |
| 185 | 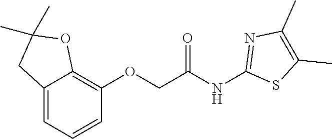 |
| 186 | 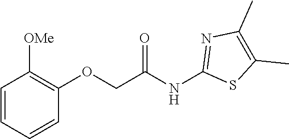 [SMILES: COc1ccccc1OCC(=O)Nc1nc(C)c(C)s1] |
| 187 | 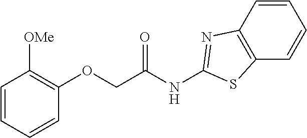 [SMILES: COc1ccccc1OCC(=O)Nc1nc2ccccc2s1] |
| 188 | 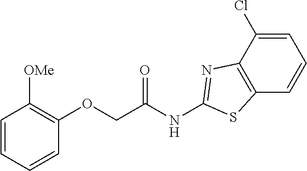 [SMILES: COc1ccccc1OCC(=O)Nc1nc2c(Cl)cccc2s1] |
| 189 | 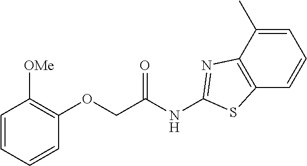 [SMILES: COc1ccccc1OCC(=O)Nc1nc2c(C)cccc2s1] |
| 190 | 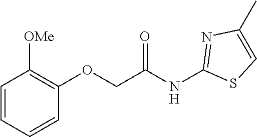 [SMILES: COc1ccccc1OCC(=O)Nc1nc(C)cs1] |
| 191 | 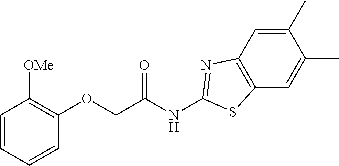 [SMILES: COc1ccccc1OCC(=O)Nc1nc2cc(C)c(C)cc2s1] |
| 192 | 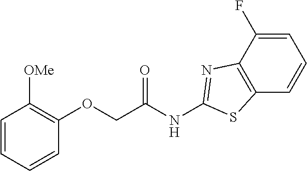 [SMILES: COc1ccccc1OCC(=O)Nc1nc2c(F)cccc2s1] |
| 193 | 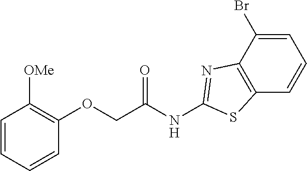 [SMILES: COc1ccccc1OCC(=O)Nc1nc2c(Br)cccc2s1] |
| 194 | 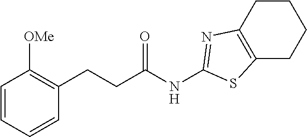 [SMILES: COc1ccccc1CCC(=O)Nc1nc2c(s1)CCCC2] |
| 195 | 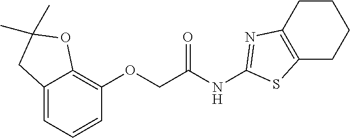 [SMILES: CC1(C)Cc2cccc(OCC(=O)Nc3nc4c(s3)CCCC4)c2O1] |
| 196 | 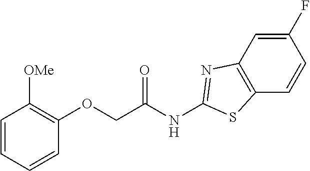 [SMILES: COc1ccccc1OCC(=O)Nc1nc2cc(F)ccc2s1] |

### Administration and Pharmaceutical Compositions

Some embodiments include pharmaceutical compositions comprising: (a) a therapeutically effective amount of a compound provided herein, or its corresponding enantiomers, diastereomers, tautomers, prodrugs, and pharmaceutically acceptable salts thereof, and (b) a pharmaceutically acceptable carrier.

The compounds provided herein may also be useful in combination (administered together or sequentially) with one or more other known agents or therapies (e.g., a second antiviral agent as described herein).

Known agents and therapies include but are not limited to antiviral agents (e.g., second antiviral agents), immune-based therapies including cell-based, antibody-based and other immunomodulators, therapeutic vaccines, latency reversing agents (LRA) and antagonists of viral proteins such as Tat or Rev.

Classes of antiviral agents include but are not limited to non-nucleoside reverse transcriptase inhibitors (NNRTIs), nucleoside reverse transcriptase inhibitors (NRTIs), protease inhibitors (PIs), fusion inhibitors, CCR5 antagonists and other entry or fusion inhibitors, maturation inhibitors, capsid inhibitors, and integrase strand transfer inhibitors (INSTIs).

Examples of antiviral agents include but are not limited to: abacavir (ABC), acyclovir, adefovir, amdoxovir (RFS Pharma), amprenavir, apricitabine (Avexa Pharmaceuticals), asunaprevir, atazanavir, ateviridine, bevirimat (Myriad Genetics), BI 224436 (Boehringer Ingelheim), bictegravir (GS-9883), BMS-955176 (Bristol Myers-Squibb), boceprevir, brecanavir (GlaxoSmithKline), brivudine, cabotegravir, capravirine (Pfizer), cenicriviroc (TBR-652, TAK-652), censavudine (Bristol Myers-Squibb), cidofovir, clevudine, cobicistat, daclatasvir, darunavir, delavirdine, dexelvucitabine (Pharmasset and Incyte), didanosine, dolutegravir, doravirine (Merck & Co.), efavirenz, elvitegravir, elvucitabine (Achillion Pharmaceuticals, Inc.), emtricitabine (FTC), emvirine, enfuvirtide (T-20), entecavir (ETV), Epivir (lamivudine), etravirine, faldaprevir, famciclovir, fosamprenavir (prodrug of amprenavir), fostemsavir (prodrug of temsavir), ganciclovir, globoidnan A, hypericin, ibalizumab (TMB-355) (being developed by Taimed Biologics), idoxuridine, indinavir, lamivudine (3TC), lasinavir (Bristol Myers-Squibb), lersivirine (UK-453061), lobucavir, lopinavir, maraviroc, methisazone, MK-2048 (Merck & Co.), moroxydine, mozenavir, nelfinavir, nevirapine, oseltamivir, penciclovir, PRO 140 (Cytodyn Inc.), racivir (Pharmasset), raltegravir, Retrovir (zidovudine), ribavirin, rilpivirine, rimantadine, ritonavir, saquinavir, simeprevir, sofosbuvir, stampidine, stavudine (d4T), telaprevir (VX-950), telbivudine, temsavir, tenofovir alafenamide fumarate, tenofovir disoproxil fumarate (TDF), tipranavir, tromantadine, umifenovir, valaciclovir, valganciclovir, vicriviroc, vidarabine, VIR-576, zalcitabine, zanamivir, and zidovudine (AZT).

Examples of immune-based therapies include but are not limited to: Aralen (chloroquine phosphate), DermaVir (being developed by Genetic Immunity), Interleukin-7 (IL-7) (being developed by Cytheris), Lexgenleucel-T (VRX-496) (being developed by VIRxSYS), Plaquenil (hydroxychloroquine), Proleukin (aldesleukin, Interleukin-2, or IL-2) (being developed by Chiron Corporation and also, Amgen Pharmaceuticals), SB-728-T (ZFP TF) (being developed by Sangamo Biosciences), TLR7 agonists (being developed by Gilead Sciences) and Vacc-4x (being developed by Bionor Pharma).

Administration of the compounds disclosed herein or the pharmaceutically acceptable salts thereof can be via any of the accepted modes of administration, including, but not limited to, orally, subcutaneously, intravenously, intranasally, topically, transdermally, intraperitoneally, intramuscularly, intrapulmonarilly, vaginally, rectally, ontologically, neuro-otologically, intraocularly, subconjuctivally, via anterior eye chamber injection, intravitreally, intraperitoneally, intrathecally, intracystically, intrapleurally, via wound irrigation, intrabuccally, intra-abdominally, intra-articularly, intra-aurally, intrabronchially, intracapsularly, intrameningeally, via inhalation, via endotracheal or endobronchial instillation, via direct instillation into pulmonary cavities, intraspinally, intrasynovially, intrathoracically, via thoracostomy irrigation, epidurally, intratympanically, intracisternally, intravascularly, intraventricularly, or intraosseously. In some embodiments, the administration method includes oral or parenteral administration.

Compounds provided herein intended for pharmaceutical use may be administered as crystalline or amorphous products. Pharmaceutically acceptable compositions may include solid, semi-solid, liquid, solutions, colloidal, liposomes, emulsions, suspensions, complexes, coacervates and aerosols. Dosage forms, such as, e.g., tablets, capsules, powders, liquids, suspensions, suppositories, aerosols, implants, controlled release or the like. They may be obtained, for example, as solid plugs, powders, or films by methods such as precipitation, crystallization, milling, grinding, supercritical fluid processing, coacervation, complex coacervation, encapsulation, emulsification, complexation, freeze drying, spray drying, or evaporative drying. Microwave or radio frequency drying may be used for this purpose. The compounds can also be administered in sustained or controlled release dosage forms, including depot injections, osmotic pumps, pills (tablets and or capsules), transdermal (including electrotransport) patches, implants and the like, for prolonged and/or timed, pulsed administration at a predetermined rate.

The compounds can be administered either alone or in combination with a conventional pharmaceutical carrier, excipient or the like. Pharmaceutically acceptable excipients include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β, and γ-cyclodextrin, or chemically modified derivatives such as hydroxyalkylcyclodextrins, including 2- and 3-hydroxypropyl-β-cyclodextrins, or other solubilized derivatives can also be used to enhance delivery of compounds described herein. Dosage forms or compositions containing a compound as described herein in the range of 0.005% to 100% with the balance made up from non-toxic carrier may be prepared. The contemplated compositions may contain 0.001%-100% of a compound provided herein, in one embodiment 0.1-95%, in another embodiment 75-85%, in a further embodiment 20-80%. Actual methods of preparing such dosage forms are known, or will be apparent, to those skilled in this art; for example, see Remington: The Science and Practice of Pharmacy, 22^{nd} Edition (Pharmaceutical Press, London, U K. 2012).

In one embodiment, the compositions will take the form of a unit dosage form such as a pill or tablet and thus the composition may contain, along with a compound provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils, PEG's, poloxamer 124 or triglycerides) is encapsulated in a capsule (gelatin or cellulose base capsule). Unit dosage forms in which one or more compounds provided herein or additional active agents are physically separated are also contemplated; e.g., capsules with granules (or tablets in a capsule) of each drug; two-layer tablets; two-compartment gel caps, etc. Enteric coated or delayed release oral dosage forms are also contemplated.

Liquid pharmaceutically administrable compositions can, for example, be prepared by dissolving, dispersing, etc. a compound provided herein and optional pharmaceutical adjuvants in a carrier (e.g., water, saline, aqueous dextrose, glycerol, glycols, ethanol or the like) to form a solution, colloid, liposome, emulsion, complexes, coacervate or suspension. If desired, the pharmaceutical composition can also contain minor amounts of nontoxic auxiliary substances such as wetting agents, emulsifying agents, co-solvents, solubilizing agents, pH buffering agents and the like (e.g., sodium acetate, sodium citrate, cyclodextrin derivatives, sorbitan monolaurate, triethanolamine acetate, triethanolamine oleate, and the like).

In some embodiments, the compositions are provided in unit dosage forms suitable for single administration.

In some embodiments, the compositions are provided in unit dosage forms suitable for twice a day administration.

In some embodiments, the compositions are provided in unit dosage forms suitable for three times a day administration.

Injectables can be prepared in conventional forms, either as liquid solutions, colloid, liposomes, complexes, coacervate or suspensions, as emulsions, or in solid forms suitable for reconstitution in liquid prior to injection. The percentage of a compound provided herein contained in such parenteral compositions is highly dependent on the specific nature thereof, as well as the activity of the compound and the needs of the patient. However, percentages of active ingredient of 0.01% to 10% in solution are employable, and could be higher if the composition is a solid or suspension, which could be subsequently diluted to the above percentages.

In some embodiments, the compositions are provided in dosage forms suitable for continuous dosage by intravenous infusion over a period of about 1-96 hours.

In some embodiments, the compositions are provided in dosage forms suitable for continuous dosage by intravenous infusion over a period of about 1-72 hours.

In some embodiments, the compositions are provided in dosage forms suitable for continuous dosage by intravenous infusion over a period of about 1-48 hours.

In some embodiments, the compositions are provided in dosage forms suitable for continuous dosage by intravenous infusion over a period of about 1-24 hours.

In some embodiments, the compositions are provided in dosage forms suitable for continuous dosage by intravenous infusion over a period of about 1-12 hours.

In some embodiments, the compositions are provided in dosage forms suitable for continuous dosage by intravenous infusion over a period of about 1-6 hours.

It is to be noted that concentrations and dosage values may also vary depending on the specific compound and the severity of the condition to be alleviated. It is to be further understood that for any particular patient, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

In one embodiment, the compositions can be administered to the respiratory tract (including nasal and pulmonary) e.g., through a nebulizer, metered-dose inhalers, atomizer, mister, aerosol, dry powder inhaler, insufflator, liquid instillation or other suitable device or technique.

In some embodiments, aerosols intended for delivery to the nasal mucosa are provided for inhalation through the nose. For optimal delivery to the nasal cavities, inhaled particle sizes of about 5 to about 100 microns are useful, with particle sizes of about 10 to about 60 microns being preferred. For nasal delivery, a larger inhaled particle size may be desired to maximize impaction on the nasal mucosa and to minimize or prevent pulmonary deposition of the administered formulation. In some embodiments, aerosols intended for delivery to the lung are provided for inhalation through the nose or the mouth. For delivery to the lung, inhaled aerodynamic particle sizes of about less than 10 μm are useful (e.g., about 1 to about 10 microns). Inhaled particles may be defined as liquid droplets containing dissolved drug, liquid droplets containing suspended drug particles (in cases where the drug is insoluble in the suspending medium), dry particles of pure drug substance, drug substance incorporated with excipients, liposomes, emulsions, colloidal systems, coacervates, aggregates of drug nanoparticles, or dry particles of a diluent which contain embedded drug nanoparticles.

In some embodiments, compounds of Formula (I) disclosed herein intended for respiratory delivery (either systemic or local) can be administered as aqueous formulations, as non-aqueous solutions or suspensions, as suspensions or solutions in halogenated hydrocarbon propellants with or without alcohol, as a colloidal system, as emulsions, coacervates, or as dry powders. Aqueous formulations may be aerosolized by liquid nebulizers employing either hydraulic or ultrasonic atomization or by modified micropump systems (like the soft mist inhalers, the Aerodose® or the AERx® systems). Propellant-based systems may use suitable pressurized metered-dose inhalers (pMDIs). Dry powders may use dry powder inhaler devices (DPIs), which are capable of dispersing the drug substance effectively. A desired particle size and distribution may be obtained by choosing an appropriate device.

Solid compositions can be provided in various different types of dosage forms, depending on the physicochemical properties of the compound provided herein, the desired dissolution rate, cost considerations, and other criteria. In one of the embodiments, the solid composition is a single unit. This implies that one unit dose of the compound is comprised in a single, physically shaped solid form or article. In other words, the solid composition is coherent, which is in contrast to a multiple unit dosage form, in which the units are incoherent.

Examples of single units which may be used as dosage forms for the solid composition include tablets, such as compressed tablets, film-like units, foil-like units, wafers, lyophilized matrix units, and the like. In one embodiment, the solid composition is a highly porous lyophilized form. Such lyophilizates, sometimes also called wafers or lyophilized tablets, are particularly useful for their rapid disintegration, which also enables the rapid dissolution of the compound.

On the other hand, for some applications the solid composition may also be formed as a multiple unit dosage form as defined above. Examples of multiple units are powders, granules, microparticles, pellets, mini-tablets, beads, lyophilized powders, and the like. In one embodiment, the solid composition is a lyophilized powder. Such a dispersed lyophilized system comprises a multitude of powder particles, and due to the lyophilization process used in the formation of the powder, each particle has an irregular, porous microstructure through which the powder is capable of absorbing water very rapidly, resulting in quick dissolution. Effervescent compositions are also contemplated to aid the quick dispersion and absorption of the compound.

Another type of multiparticulate system which is also capable of achieving rapid drug dissolution is that of powders, granules, or pellets from water-soluble excipients which are coated with a compound provided herein so that the compound is located at the outer surface of the individual particles. In this type of system, the water-soluble low molecular weight excipient may be useful for preparing the cores of such coated particles, which can be subsequently coated with a coating composition comprising the compound and, for example, one or more additional excipients, such as a binder, a pore former, a saccharide, a sugar alcohol, a film-forming polymer, a plasticizer, or other excipients used in pharmaceutical coating compositions.

In some embodiments, the compounds provided herein are formulated as a vaccine adjuvant.

Also provided herein are kits. Typically, a kit includes one or more compounds or compositions as described herein. In certain embodiments, a kit can include one or more delivery systems, e.g., for delivering or administering a compound as provided herein, and directions for use of the kit (e.g., instructions for treating a patient).

### Methods of Treatment

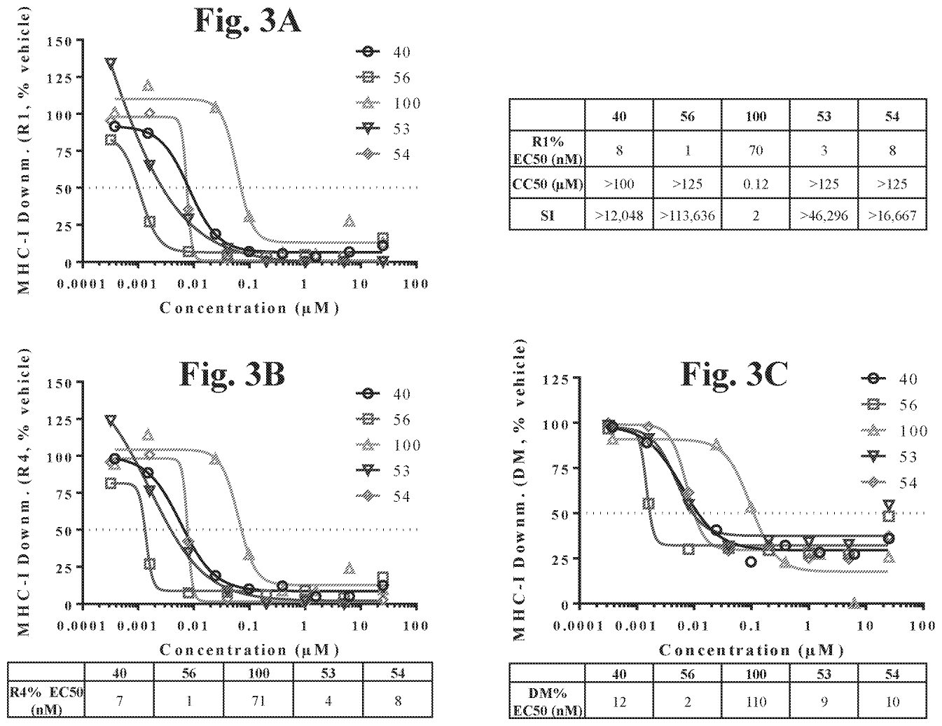

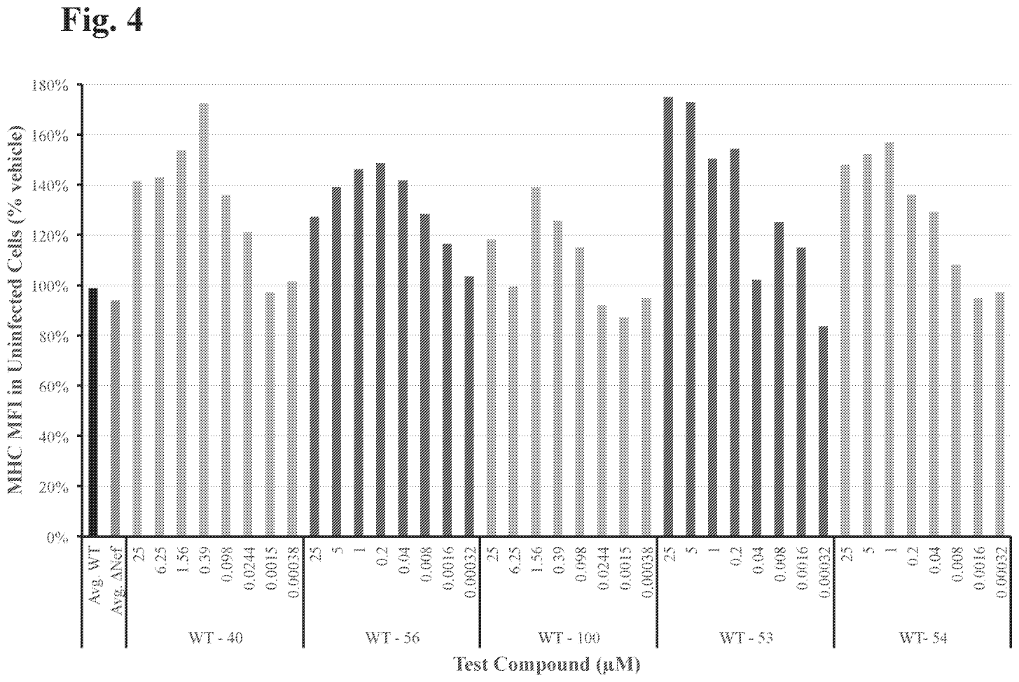

The inhibitors of the present disclosure inhibit and/or reduce the downmodulation of MIC-I in viral infected cells (e.g., HIV-infected cells), and also enhance the surface expression of MHC-I in uninfected cells (as shown in FIGS. 3 and 4, respectively). Similar results have been observed in other cells of both human (e.g. Hut78, Jurkat, A3.01, H9, HSB-2) and murine origin (B16-F10).

Many viruses have evolved strategies to escape the immune system by interfering with the viral antigen presentation machinery. Viruses from diverse families, including the retroviridae, herpesviridae, adenoviridae and papilomaviridae interfere with the MHC class I antigen presentation pathway. Viruses block the translocation of peptides by the transporter associated with antigen processing (TAP), and also cause the degradation or mislocalization of MHC class I molecules [Immunology (2003) 110 163-169]. In HIV-infected cells, the viral protein Nef downmodulates MHC-I from the cell surface. Nef binds to the intracellular domain of MHC-I and also to proteins involved in the trafficking of many other cellular proteins [Scientific Reports (2016), 6, 37021; Advances in Virus Research (2011), 80, 103-127; Nature (1998), 391, 397-401]. By doing this, Nef enhances the internalization of MHC-I and its degradation into lysosomes, and also interferes with its intracellular transport to the cell surface [Journal of Virology (2003), 77, 3041-3049; Virology (2001), 282, 267-277; Journal of Virology (2005), 79, 632-636]. By removing MIC-I from the cell surface Nef helps HIV to escape immune recognition by CD8-positive T cells [Nature (1998), 391, 397-401]. These CD8 cells detect viral antigens bound to MHC-I on the surface of infected cells, an effect known as “antigen presentation”. Therefore, it has been hypothesized that in vivo inhibition of MIC-I downmodulation may restore viral antigen presentation and efficient recognition of HIV-infected cells by CD8-positive T cells in vivo [Molecular biology of the cell (2010), 21, 3279-3292; Immunological reviews (1999), 168, 65-74].

Accordingly, provided herein are methods of treating a viral infection in a patient, the method comprising administering to the patient an effective amount of a compound according to Formulas I, II, III, IV, V, and VI.

Other embodiments disclosed herein include methods of inhibiting or reducing viral mediated downmodulation of MHC-I by administering to a patient (e.g., a patient infected by a primate lentivirus, such as HIV-1, HIV-2 and SIV), an effective amount of a compound according to Formulas I, II, III, IV, V, and VI. For example, the compounds and compositions provided herein can be used to treat HIV-1, HIV-2 and SIV.

In infected individuals some HIV infected cells remain in a state of latency in which the HIV genome is inserted into the host chromosome, but it remains transcriptionally inactive [Journal of Clinical Investigation (2016), 126(2), 448-454]. In these latent cells no viral antigens are expressed and therefore latently infected cells remain unrecognized by CD8-positive T cells. Latently HIV-infected cells can live for decades and persist even in the presence of active anti-retroviral therapy (ART) [Science (1997), 278, 1295-1300]. Highly active ART may lower the HIV viral load in patients below undetectable levels. However, latent cells still persist and can be reactivated later to reinitiate virus replication. The existence of latently infected cells precludes complete viral eradication even in the presence of successful ART [Frontiers in Immunology (2015), 6, 505].

Latency reactivating agents (LRA) have been proposed for HIV eradication approaches to activate transcription of HIV and reactivate virus replication in latent cells [Acta Pharmacologica Sinica (2015), 36, 908-916]. Unfortunately, reactivated cells often fail to be recognized and cleared by HIV-specific CD8-positive T cells [Immunity (2012), 36(3), 491-501]. The inhibitors of the present disclosure could act as immunomodulators to enhance the response against HIV mediated by CD8-positive cells. Compounds of the present disclosure could be used in HIV cure approaches in which patients, who are undergoing ART and display undetectable or low viral load, are then treated with one or several LRAs to first reactivate the latently infected cells. During or after the treatment with LRAs, the patients are treated with the MHC-I downmodulation inhibitors of the present disclosure to increase viral antigen presentation in reactivated cells and promote their recognition by the own patient's immune cells. This approach could include treatment with one or several antiretrovirals to ensure that viral replication is controlled after reactivation with LRAs. HIV cure approaches could also include other immunotherapies together with the treatment with compounds of the present disclosure. Among these other immunotherapies could be the use of antibodies or bi-specific antibodies against HIV and or immune proteins, the use of therapeutic vaccines, the use of the patient's immune cells treated or reprogrammed (in vivo or ex-vivo) using technologies such as chimeric antigen receptors or cytokine treatments to augment their immune recognition activity or specificity towards HIV, or alternatively to use heterologous immune cells selected from other patients who manage to clear or control HIV infection (e.g. CD8-positive cells from HIV long-term nonprogressors) or immune cells treated or reprogrammed to display a more potent or specific immune activity against HIV-infected cells.

Accordingly, provided herein are methods of treating a viral infection in a patient, the method comprising administering to the patient an effective amount of a compound according to Formulas I, II, III, IV, V, and VI, or a pharmaceutically acceptable salt thereof, and an effective amount of a latency-reversing agent (LRA). The LRA can be administered before, after, or concurrently with the compound according to Formulas I, II, III, IV, V, and VI. In some embodiments, the LRA is administered before administration of the compound according to Formulas I, II, III, IV, V, and VI.

Further provided herein is a method of treating a viral infection in a patient previously administered a latency-reversing agent (LRA), the method comprising administering to the patient an effective amount of a compound according to Formulas I, II, III, IV, V, and VI, or a pharmaceutically acceptable salt thereof.

In some embodiments, the methods of the present disclosure involve the use of one or more compounds of the present disclosure in the inhibition or reduction of the HIV mediated downmodulation of major histocompatibility complex I (MHC-I), the prophylaxis treatment or eradication of infection by human immunodeficiency virus (HIV) and the prophylaxis, treatment or delay in the onset or progression of consequent pathological conditions such as AIDS. Prophylaxis of AIDS, treating AIDS, delaying the onset or progression of AIDS, or treating or prophylaxis of infection by HIV is defined as including, but not limited to, treatment of a wide range of states of HIV infection: AIDS and ARC (AIDS related complex), both symptomatic and asymptomatic. For example, the present disclosure can be employed to treat infection by HIV after suspected past exposure to HIV by such means as blood transfusion, exchange of body fluids, bites, accidental needle stick, or exposure to a patient's blood, for example, during surgery.

The compounds of the present disclosure also display a potent anti-retroviral effect when tested with in vitro assays of HIV infection. In some embodiments, the compounds are used in anti-HIV therapies alone or in combination other antiretrovirals. Such antiretrovirals (e.g., second antiviral agents) can include those of known mechanism (entry inhibitors, fusion inhibitors, protease inhibitors, reverse transcriptase or integrase inhibitors). In some embodiments, an antiretroviral agent is a nucleoside reverse transcriptase inhibitor, a non-nucleoside reverse transcriptase inhibitor, a protease inhibitor, a maturation inhibitor, a capsid inhibitor, a fusion inhibitor, an entry inhibitor, an integrase inhibitor, a co-receptor antagonist, a viral adsorption inhibitor, a viral specific transcription inhibitor, a cyclin dependent kinase inhibitor, or a combination thereof. In some embodiments, the antiretroviral is one or more of those described herein.

In some embodiments, a compound of Formulas I, II, III, IV, V, and/or VI can be used as an adjuvant added to a vaccine to increase the body's immune response to the vaccine.

In some embodiments, a compound of Formulas I, II, III, IV, V, and/or VI can be added to a vaccine to increase the efficiency of a vaccine against infectious agents (e.g., to be added as an adjuvant).

In some embodiments, the disclosure provides a method for inhibiting or reducing the HIV-induced downmodulation of MHC-I in infected cells.

In some embodiments, the disclosure provides a method for inhibiting or reducing viral-induced downmodulation of MHC-I in infected cells.

In some embodiments, the disclosure provides a method for increasing surface expression of MHC-I in uninfected cells.

In some embodiments, the disclosure provides a method for increasing surface expression of MHC-I in infected cells.

In some embodiments, the disclosure provides a method for treating a viral infection.

In some embodiments, the disclosure provides a method for treating a viral infection by increasing the surface expression of MHC-I in infected cells.

In some embodiments, the disclosure provides a method for treating a viral infection by inhibiting or reducing viral-induced downmodulation of MHC-I in infected cells.

In some embodiments, the disclosure provides a method for increasing viral antigen presentation.

In some embodiments, the disclosure provides a method for of treating or ameliorating in a patient a viral infection by increasing viral antigen presentation.

In some embodiments, the disclosure provides a method for treating an infection caused by a virus which induces downmodulation of MHC-I or prevents the synthesis or transport of MHC-I to the cell surface.

In some embodiments, the virus can be selected from the group consisting of: members of the Retroviridae family such as human immunodeficiency virus 1 (HIV-1), human immunodeficiency virus 2 (HIV-2), simian immunodeficiency virus (SIV), human T-cell lymphotropic virus type 1 (HTLV-I) and type II (HTLV-II); members of the Herpesviridae family, including herpes simplex virus 1 (HSV-1), herpes simplex virus 2 (HSV-2), cytomegalovirus (CMV) (e.g., human CMV or HCMV), Epstein-Barr virus (EBV), varicella zoster virus (VZV), Kaposi's sarcoma associated virus (KSHV) and human herpesvirus 6A (HHV-6A), 6B (HHV-6B) and 7 (HHV-7); members of the Adenoviridae family including human adenovirus type 1 (HAdV-1 to 67 in species A to G), and members of the Papillomaviridae, including over 170 types of the human papillomaviruses (HPV).

In some embodiments, the disclosure provides a method of treating in a patient a disorder or disease selected from the group consisting of: human immunodeficiency virus 1 (HIV-1), human immunodeficiency virus 2 (HIV-2) and simian immunodeficiency virus (SIV), the method comprising administering to the patient a therapeutically effective amount of a compound of Formulas I, II, III, IV, V, and VI.

### Evaluation of Biological Activity

The biological activity of the compounds described herein can be tested using any suitable assay known to those of skill in the art. For example, the activity of a compound may be tested using one or more of the methods outlined below using primary or transformed cells permissive to HIV infection and in which MHC-I is downmodulated upon infection, or alternatively monitoring the extent of MHC-I downmodulation in cells transduced with or expressing the HIV nef gene.

To further illustrate this disclosure, the following examples are included. The examples should not, of course, be construed as specifically limiting the disclosure. Variations of these examples within the scope of the claims are within the purview of one skilled in the art and are considered to fall within the scope of the disclosure as described, and claimed herein. The reader will recognize that the skilled artisan, armed with the present disclosure, and skill in the art is able to prepare and use the disclosure without exhaustive examples.

### EXAMPLES

### Compound Preparation

In some cases, the starting materials used in preparing the compounds of the disclosure are known, made by known methods, or are commercially available. It will be apparent to the skilled artisan that methods for preparing precursors and functionality related to the compounds claimed herein are, in some cases, generally described in the literature. The skilled artisan given the literature and this disclosure is well equipped to prepare any of the compounds.

It is recognized that the skilled artisan in the art of organic chemistry can readily carry out manipulations without further direction, that is, it is well within the scope and practice of the skilled artisan to carry out these manipulations. These include reduction of carbonyl compounds to their corresponding alcohols, oxidations, acylations, aromatic substitutions, both electrophilic and nucleophilic, etherifications, esterification and saponification and the like. These manipulations are discussed in standard texts such as March's Advanced Organic Chemistry: Reactions, Mechanisms, and Structure 7^{th} Ed., John Wiley & Sons (2013), Carey and Sundberg, Advanced Organic Chemistry 5^{th} Ed., Springer (2007), Comprehensive Organic Transformations: A Guide to Functional Group Transformations, 2^{nd} Ed., John Wiley & Sons (1999) (incorporated herein by reference in its entirety) and the like.

The skilled artisan will readily appreciate that certain reactions are best carried out when other functionality is masked or protected in the molecule, thus avoiding any undesirable side reactions and/or increasing the yield of the reaction. Often the skilled artisan utilizes protecting groups to accomplish such increased yields or to avoid the undesired reactions. These reactions are found in the literature and are also well within the scope of the skilled artisan. Examples of many of these manipulations can be found for example in P. Wuts Greene's Protective Groups in Organic Synthesis, 5th Ed., John Wiley & Sons (2014), incorporated herein by reference in its entirety.

Trademarks used herein are examples only and reflect illustrative materials used at the time of the disclosure. The skilled artisan will recognize that variations in lot, manufacturing processes, and the like, are expected. Hence the examples, and the trademarks used in them are non-limiting, and they are not intended to be limiting, but are merely an illustration of how a skilled artisan may choose to perform one or more of the embodiments of the disclosure.

(¹H) nuclear magnetic resonance spectra (NMR) were measured in the indicated solvents on either a Bruker NMR spectrometer (Avance™ DRX500, 500 MHz for ¹H) or a Bruker (400 MHz for ¹H). Peak positions are expressed in parts per million (ppm) downfield from tetramethylsilane. The peak multiplicities are denoted as follows, s, singlet; d, doublet; t, triplet; q, quartet; ABq, AB quartet; quin, quintet; sex, sextet; sep, septet; non, nonet; dd, doublet of doublets; ddd, doublet of doublets of doublets; d/ABq, doublet of AB quartet; dt, doublet of triplets; td, triplet of doublets; dq, doublet of quartets; m, multiplet.

The following abbreviations have the indicated meanings:
APC=allophycocyanin
ART=anti retroviral therapy
brine=saturated aqueous sodium chloride
CDCl3=deuterated chloroform
DCM=dichloromethane
   DMSO=dimethylsulfoxide
   DMF=N, N-dimethylformamide
   ESIMS=electrospray ionization mass spectrometry
   m/z=mass-to-charge ratio
   EtOAc=ethyl acetate
   GFP=green fluorescent protein
   HIV=human immunodeficiency virus
   HLA=human leukocyte antigen
   LAH=lithium aluminum hydride
   LRA=latency reversing agent
   MHC-I=major histocompatibility complex I
   NMR=nuclear magnetic resonance
   rt=room temperature
   THE=tetrahydrofuran
   DIEA=N,N-diisopropylethylamine
   DEAD=diethyl azodicarboxylate
   satd.=saturated
   h=hours
   min=minutes
   HATU=1-[bis(dimethylamino)methylene]-1H-1,2,3-triazolo[4,5-b]pyridinium 3-oxid hexafluorophosphate
   Ghosez's reagent=1-chloro-N,N,2-trimethyl-1-propenylamine
   Xantphos=4,5-bis(diphenylphosphino)-9,9-dimethylxanthene
   Pd2(dba)₃=tris(dibenzylideneacetone)dipalladium(0)
   tBuBrettPhos=2-(di-tert-butylphosphino)-2′,4′,6′-triisopropyl-3,6-dimethoxy-1,1′-biphenyl
   Pd(dppf)₂Cl₂.DCM=[1,1′-bis(diphenylphosphino)ferrocene]
   dichloropalladium(II), complex with dichloromethane
   Pd—C=palladium on carbon

The following example schemes are provided for the guidance of the reader, and collectively represent an example method for making the compounds provided herein. Furthermore, other methods for preparing compounds of the disclosure will be readily apparent to the person of ordinary skill in the art in light of the following reaction schemes and examples. The skilled artisan is thoroughly equipped to prepare these compounds by those methods given the literature and this disclosure. The compound numberings used in the synthetic schemes depicted below are meant for those specific schemes only, and should not be construed as or confused with same numberings in other sections of the application. Unless otherwise indicated, all variables are as defined above.

### General Procedures

Compounds of Formula I of the present disclosure can be prepared as depicted in Scheme 1.

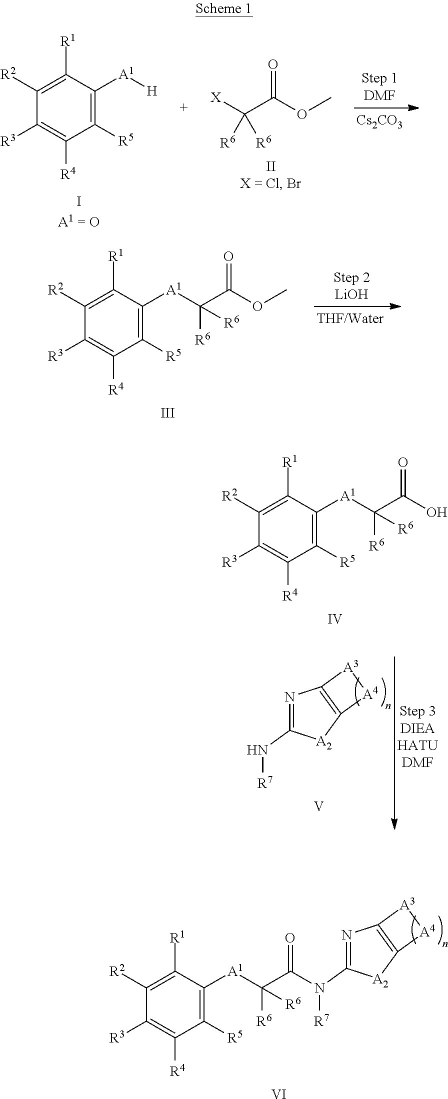

Scheme 1 describes a method for preparation of phenoxyacetamide derivatives (VI) by first forming reaction of a phenol (I) with a 2-haloacetyl ester (II), for example, methyl bromoacetate, mediated by a base, for example cesium carbonate, to form intermediate III. The ester (III) can be hydrolyzed to the corresponding acid (IV) with a base, for example LiOH. Reaction of IV with the heteroaryl amine (V) mediated by an amide coupling reagent, for example DIEA and HATU is used to produce the desired phenoxyacetamide derivatives (VI). Alternative coupling conditions, for example, Ghosez's reagent with pyridine in DCM can also be utilized. These methods can also be utilized in the preparation of Formulas II, III, IV, V, and VI.

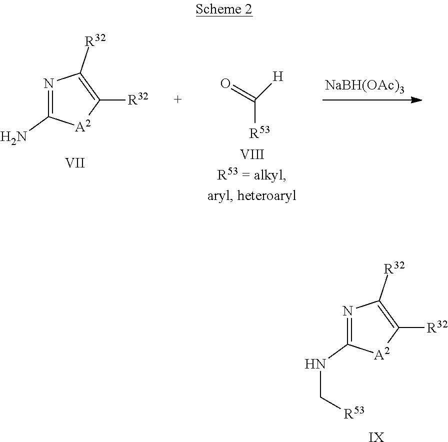

Scheme 2 describes a method for preparation of a secondary amine by reaction of a heteroaryl amine, for example, an aminothiazole (VII) and an aldehyde, for example, 2-pyridyl aldehyde (VIII) with a reducing agent, for example sodium triacetoxyborohydride, to produce the desired secondary amine (IX). Alternative reducing conditions, for example, sodium borohydride in methanol, can also be utilized.

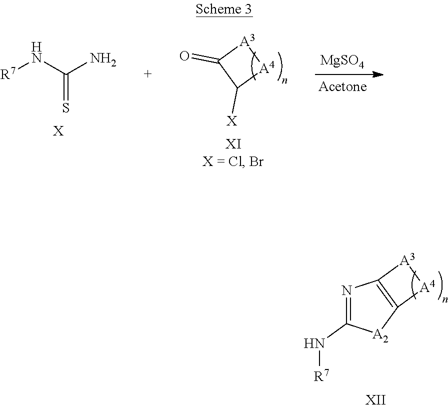

Scheme 3 describes a method for preparation of a 2-aminothiazole. Reaction of a substituted thiourea (X), for example, methyl thiourea, with a haloketone, for example chlorocyclohexanone, mediated by MgSO4 in refluxing acetone is used to produce the desired thiazole (XII).

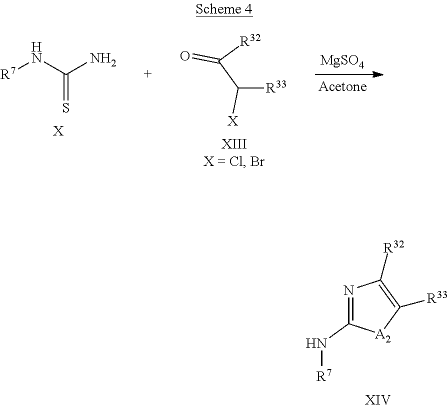

Scheme 4 describes a method for preparation of an alternative 2-aminothiazole. Reaction of a substituted thiourea (X), for example, methyl thiourea, with a haloketone (XIII), for example chloroacetone, mediated by MgSO4 in refluxing acetone is used to produce the desired thiazole (XIV).

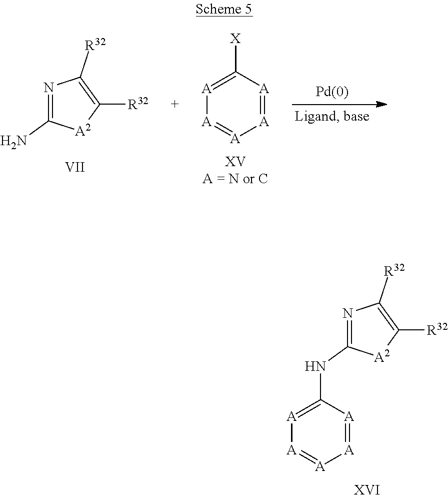

Scheme 5 describes a method for preparation of N-aryl or N-heteroaryl amines. A mixture of an aryl or heteroaryl amine, for example, an aminothiazole (VII) and an aryl halide or heteroaryl halide, for example, 2-chloropyrazine, is subjected to Pd(0), for example, Pd₂(dba)₃, and a ligand, for example xantphos, and a base, for example sodium carbonate, to produce the desired amine (XVI). Alternative ligand conditions, for example, tBuBrettPhos and potassium carbonate, can be utilized. Alternative coupling conditions, using CuI and potassium t-butoxide, and be utilized.

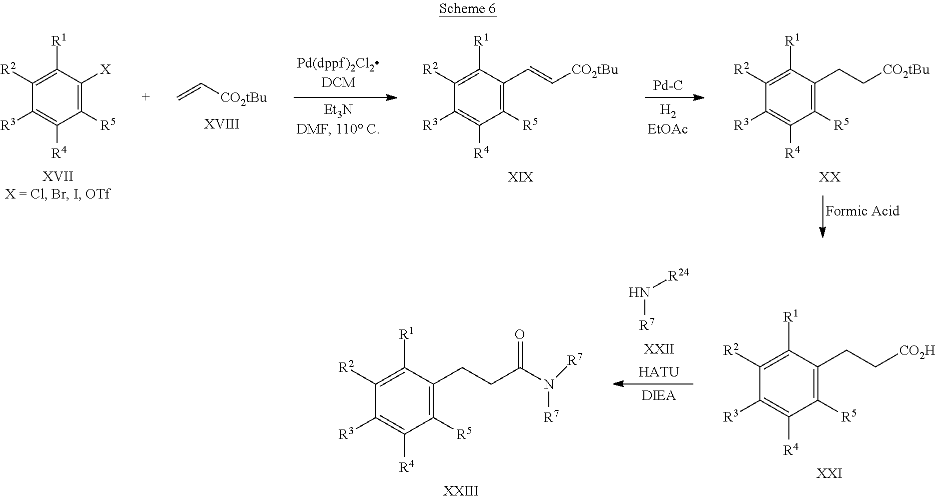

Scheme 6 describes the synthesis of propanamide derivatives. To a mixture of an aryl halide or pseudohalide (XVII), for example, an aryl triflate and an electron-deficient olefin (XVIII), for example, t-butyl acrylate, in DMF is added sequentially triethylamine and Pd(dppf)₂Cl₂.DCM, and the mixture is heated to form intermediate XIX. Olefin XIX is reacted with a catalyst, for example, Pd—C and a reductant, for example hydrogen gas, to form intermediate XX. Ester XX is reacted with an acid, for example formic acid, to form acid XXI. Reaction of XXI with the heteroaryl amine (XXII) mediated by an amide coupling reagent, for example DIEA and HATU is used to produce the desired phenoxyacetamide derivatives (XXIII). Alternative coupling conditions, for example, Ghosez's reagent with pyridine in DCM can also be utilized. This Scheme can be used in the preparation of Formulas I, II, III, IV, V and VI where A¹ is carbon.

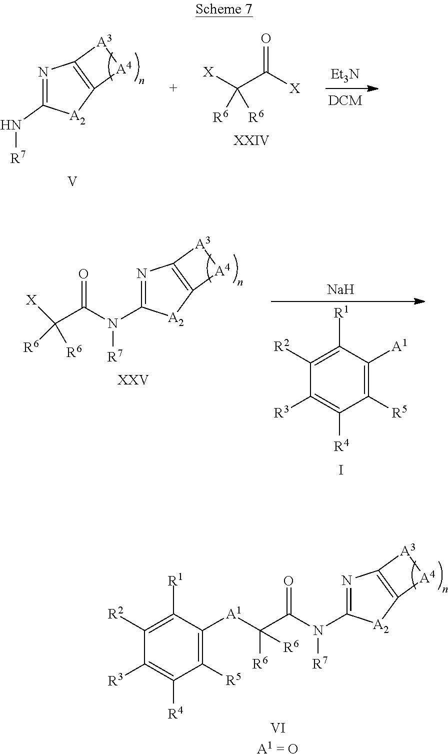

Scheme 7 describes an alternative phenoxyacetamide synthesis. An arylamine is reacted with a haloacetyl halide (XXIV), for example bromoacetyl bromide, and a base, for example triethylamine, to form haloacetamide XXV. Intermediate XXV is reacted with a phenol (I) and a base, for example sodium hydride, to form desired compound VI. This Scheme can be used in the preparation of Formulas I, II, III, IV, V and VI.

### ILLUSTRATIVE COMPOUND EXAMPLES

### Example 1

The preparation of N-(4-bromobenzo[d]thiazol-2-yl)-2-((2,2-dimethyl-2,3-dihydrobenzofuran-7-yl)oxy)acetamide (1) is depicted below in Scheme 8.

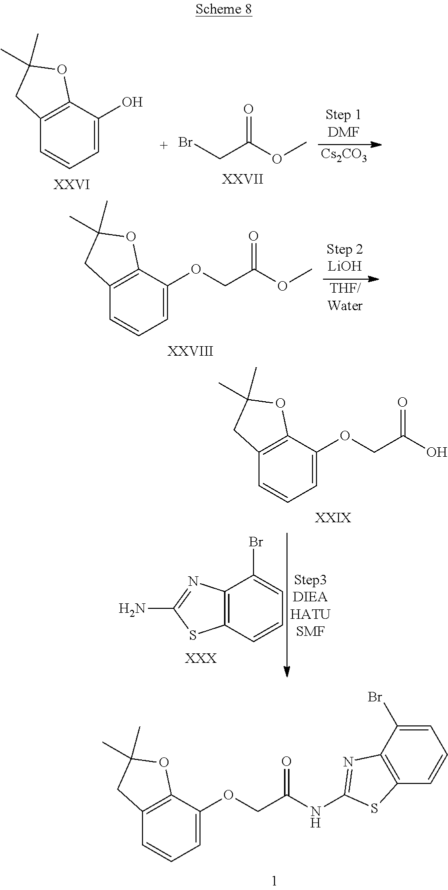

### Steps 1 and 2

A suspension of 2,2-dimethyl-2,3-dihydrobenzofuran-7-ol (XXVI) (9.50 g, 57.9 mmol), methyl bromoacetate (XXVII) (8.85 g, 57.9 mmol), cesium carbonate (37.7 g, 115.7 mmol) in DMF (95 mL) was stirred for 18 hours at rt. The reaction mixture was filtered through a pad of Celite® and the filtered cake was washed with EtOAc (1000 mL). To the filtrate was added H₂O (1000 mL). The layers were separated. The organic layer was washed with brine, dried over MgSO4, filtered, and concentrated under reduced pressure to afford methyl 2-((2,2-dimethyl-2,3-dihydrobenzofuran-7-yl)oxy)acetate (XXVIII), which was dissolved in THE (80 mL, 0.43 M solution). Lithium hydroxide (10.2 g, 426 mmol) in H₂O (80 mL) was added, and the mixture was stirred for 6 hours. The solution was diluted with water (200 mL) and washed with ether (2×100 mL). The aqueous layer was acidified to pH 3 with a solution of aq. HCl, then extracted with EtOAc (3×150 mL). The combined organic layer was washed with brine (300 mL), dried over MgSO4, filtered, and concentrated under reduced pressure. Recrystallization (EtOAc:hexanes 1:1) afforded 2-((2,2-dimethyl-2,3-dihydrobenzofuran-7-yl)oxy)acetic acid (XXIX) as a white solid (10.1 g, 45.4 mmol, 79%). ESIMS found for C₁₂H₁₄O₄: m/z 223.1 (M+1).

### Step 3

To a mixture of 2-((2,2-dimethyl-2,3-dihydrobenzofuran-7-yl)oxy)acetic acid (890 mg, 4.00 mmol) and 4-bromobenzo[d]thiazol-2-amine (XXX) (1.01 g, 4.41 mmol) in DMF (4.4 mL) was added DIEA (1.30 g, 10.0 mmol), then HATU (1.68 g, 4.41 mmol). After LCMS analysis determined the reaction to be complete, the mixture was diluted with EtOAc (150 mL) then washed with water (3×50 mL). The organic layer was washed sequentially with sat. NaHCO₃ (50 mL), and brine (50 mL). The organic layer was dried (MgSO4), filtered and concentrated, and the crude residue was purified by column chromatography (EtOAc:hexanes, gradient elution) to afford 0.92 g (53%) of N-(4-bromobenzo[d]thiazol-2-yl)-2-((2,2-dimethyl-2,3-dihydrobenzofuran-7-yl)oxy)acetamide (1). ¹H NMR (400 MHz, CHLOROFORM-d) δ ppm 1.57 (s, 6H), 3.07 (s, 2H), 4.84 (s, 2H), 6.75-6.85 (m, 2H), 6.92 (dd, J=7.03, 1.16 Hz, 1H), 7.20 (t, J=7.89 Hz, 1H), 7.66 (dd, J=7.76, 0.92 Hz, 1H), 7.78 (dd, J=7.95, 0.86 Hz, 1H), 10.47 (br s, 1H); ESIMS found for C₁₉H₁₇BrN₂O₃S: m/z 433.2 (M+1).

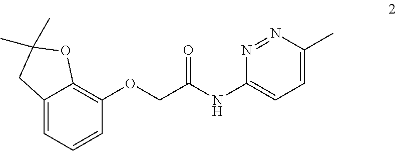

2-((2,2-Dimethyl-2,3-dihydrobenzofuran-7-yl)oxy)-N-(6-methylpyridazin-3-yl)acetamide (2) was prepared in accordance with the procedures described in Scheme 8. 56% yield. ¹H NMR (400 MHz, CHLOROFORM-d) δ ppm 1.53 (s, 6H), 2.69 (s, 3H), 3.06 (s, 2H), 4.75 (s, 2H), 6.73-6.86 (m, 2H), 6.89 (d, J=6.97 Hz, 1H), 7.36 (d, J=9.05 Hz, 1H), 8.42 (d, J=9.17 Hz, 1H), 9.56 (br s, 1H); ESIMS found for C₁₇H₁₉N₃O₃: m/z 314.1 (M+1).

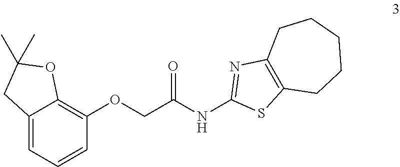 [SMILES: CC1(C)Cc2cccc(OCC(=O)Nc3nc4c(s3)CCCCC4)c2O1]

2-((2,2-Dimethyl-2,3-dihydrobenzofuran-7-yl)oxy)-N-(5,6,7,8-tetrahydro-4H-cyclohepta[d]thiazol-2-yl)acetamide (3) was prepared in accordance with the procedures described in Scheme 8. 17% yield. ¹H NMR (400 MHz, CHLOROFORM-d) δ ppm 1.52 (s, 6H), 1.65-1.77 (m, 4H), 1.81-1.89 (m, 2H), 2.71-2.77 (m, 2H), 2.79-2.85 (m, 2H), 3.04 (s, 2H), 4.75 (s, 2H), 6.75 (s, 1H), 6.76 (s, 1H), 6.84-6.89 (m, 1H), 9.74 (br s, 1H); ESIMS found for C₂₀H₂₄N₂O₃S: m/z 373.1 (M+1).

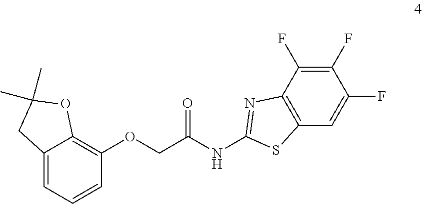 [SMILES: CC1(C)Cc2cccc(OCC(=O)Nc3nc4c(F)c(F)c(F)cc4s3)c2O1]

2-((2,2-Dimethyl-2,3-dihydrobenzofuran-7-yl)oxy)-N-(4,5,6-trifluorobenzo [d]thiazol-2-yl)acetamide (4) was prepared in accordance with the procedures described in Scheme 8. 17% yield. ¹H NMR (400 MHz, CHLOROFORM-d) δ ppm 1.55 (s, 6H), 3.06 (s, 2H), 4.83 (s, 2H), 6.76-6.85 (m, 2H), 6.91 (dt, J=6.79, 0.83 Hz, 1H), 7.42 (ddd, J=8.77, 6.45, 2.14 Hz, 1H), 10.45 (br s, 1H); ESIMS found for C₁₉H₁₅F₃N₂O₃S: m/z 409.2 (M+1).

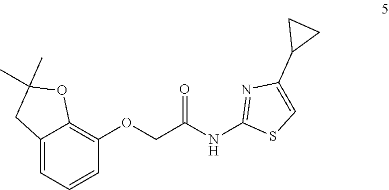 [SMILES: CC1(C)Cc2cccc(OCC(=O)Nc3nc(C4CC4)cs3)c2O1]

N-(4-Cyclopropylthiazol-2-yl)-2-((2,2-dimethyl-2,3-dihydrobenzofuran-7-yl)oxy)acetamide (5) was prepared in accordance with the procedures described in Scheme 8. 23% yield. ¹H NMR (400 MHz, CHLOROFORM-d) δ ppm 0.77-0.86 (m, 2H), 0.86-0.96 (m, 2H), 1.54 (s, 6H), 1.92-2.03 (m, 1H), 3.06 (s, 2H), 4.77 (s, 2H), 6.53 (s, 1H), 6.72-6.84 (m, 2H), 6.89 (d, J=6.60 Hz, 1H), 10.04 (br s, 1H); ESIMS found for C₁₈H₂₀N₂O₃S: m/z 345.3 (M+1).

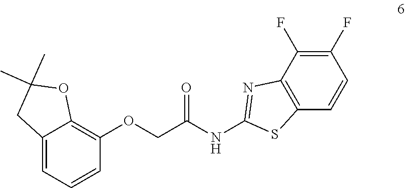 [SMILES: CC1(C)Cc2cccc(OCC(=O)Nc3nc4c(F)c(F)ccc4s3)c2O1]

N-(4,5-Difluorobenzo[d]thiazol-2-yl)-2-((2,2-dimethyl-2,3-dihydrobenzofuran-7-yl)oxy)acetamide (6) was prepared in accordance with the procedures described in Scheme 8. 17% yield. ¹H NMR (400 MHz, CHLOROFORM-d) δ ppm 1.50 (s, 6H), 3.05 (s, 2H), 4.72 (s, 2H), 6.76-6.85 (m, 2H), 6.90 (dd, J=6.66, 1.04 Hz, 1H), 7.40 (ddd, J=9.05, 6.97, 2.32 Hz, 1H), 8.35 (ddd, J=9.02, 6.88, 2.08 Hz, 1H), 9.07 (br s, 1H); ESIMS found for C₁₉H₁₆F₂N₂O₃S: m/z 391.4 (M+1).

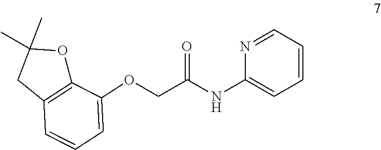 [SMILES: CC1(C)Cc2cccc(OCC(=O)Nc3ccccn3)c2O1]

2-((2,2-Dimethyl-2,3-dihydrobenzofuran-7-yl)oxy)-N-(pyridin-2-yl) acetamide (7) was prepared in accordance with the procedures described in Scheme 8. 27% yield. ¹H NMR (400 MHz, CHLOROFORM-d) δ ppm 1.52 (s, 6H), 3.05 (s, 2H), 4.72 (s, 2H), 6.72-6.85 (m, 2H), 6.88 (d, J=7.09 Hz, 1H), 7.04-7.13 (m, 1H), 7.68-7.77 (m, 1H), 8.27 (d, J=8.44 Hz, 1H), 8.32 (dd, J=4.89, 0.86 Hz, 1H), 9.11 (br s, 1H); ESIMS found for C₁₇H₁₈N₂O₃: m/z 299.3 (M+1).

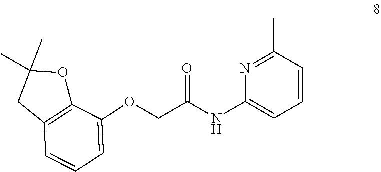 [SMILES: Cc1cccc(NC(=O)COc2cccc3c2OC(C)(C)C3)n1]

2-((2,2-Dimethyl-2,3-dihydrobenzofuran-7-yl)oxy)-N-(6-methylpyridin-2-yl) acetamide (8) was prepared in accordance with the procedures described in Scheme 8. 12% yield. ¹H NMR (400 MHz, CHLOROFORM-d) δ ppm 1.53 (s, 6H), 2.47 (s, 3H), 3.05 (s, 2H), 4.71 (s, 2H), 6.73-6.81 (m, 1H), 6.81-6.86 (m, 1H), 6.88 (d, J=7.09 Hz, 1H), 6.93 (d, J=7.46 Hz, 1H), 7.61 (t, J=7.89 Hz, 1H), 8.07 (d, J=8.31 Hz, 1H), 9.06 (br s, 1H); ESIMS found for C₁₈H₂₀N₂O₃: m/z 313.2 (M+1).

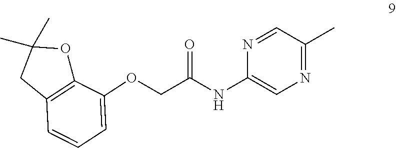

2-((2,2-Dimethyl-2,3-dihydrobenzofuran-7-yl)oxy)-N-(5-methylpyrazin-2-yl)acetamide (9) was prepared in accordance with the procedures described in Scheme 8. 25% yield. ¹H NMR (400 MHz, CHLOROFORM-d) δ ppm 1.51 (s, 6H), 2.55 (s, 3H), 3.04 (s, 2H), 4.73 (s, 2H), 6.74-6.84 (m, 2H), 6.88 (dd, J=7.03, 1.04 Hz, 1H), 8.15 (s, 1H), 9.10 (br s, 1H), 9.46 (d, J=1.22 Hz, 1H); ESIMS found for C₁₇H₁₉N₃O₃: m/z 314.2 (M+1).

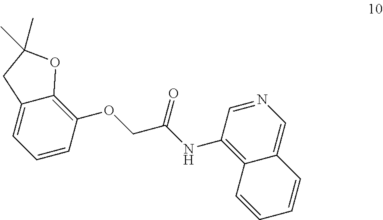 [SMILES: CC1(C)Cc2cccc(OCC(=O)Nc3cncc4ccccc34)c2O1]

2-((2,2-Dimethyl-2,3-dihydrobenzofuran-7-yl)oxy)-N-(isoquinolin-4-yl) acetamide (10) was prepared in accordance with the procedures described in Scheme 8 in 12% yield. ESIMS found for C₂₁H₂₀N₂O₃: m/z 349.2 (M+1).

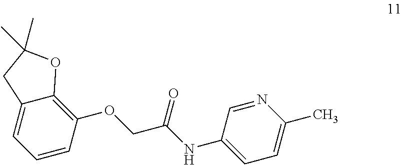

2-((2,2-Dimethyl-2,3-dihydrobenzofuran-7-yl)oxy)-N-(6-methylpyridin-3-yl) acetamide (11) was prepared in accordance with the procedures described in Scheme 8 in 21% yield. ESIMS found for C₁₈H₂₀N₂O₃: m/z 313.3 (M+1).

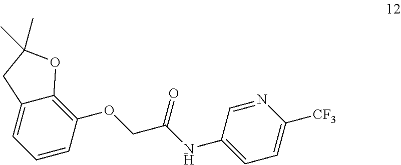 [SMILES: CC1(C)Cc2cccc(OCC(=O)Nc3ccc(C(F)(F)F)nc3)c2O1]

2-((2,2-Dimethyl-2,3-dihydrobenzofuran-7-yl)oxy)-N-(6-(trifluoromethyl) pyridin-3-yl)acetamide (12) was prepared in accordance with the procedures described in Scheme 8 in 6.1% yield. ESIMS found for C₁₈H₁₇F₃N₂O₃: m/z 367.3 (M+1).

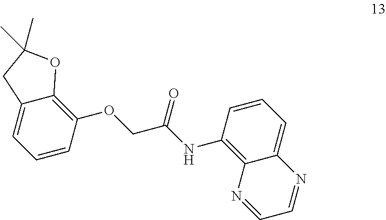 [SMILES: CC1(C)Cc2cccc(OCC(=O)Nc3cccc4nccnc34)c2O1]

2-((2,2-Dimethyl-2,3-dihydrobenzofuran-7-yl)oxy)-N-(quinoxalin-5-yl) acetamide (13) was prepared in accordance with the procedures described in Scheme 8 in 6% yield. ESIMS found for C₂₀H₁₉N₃O₃: m/z 350.3 (M+1).

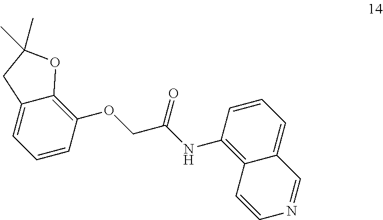 [SMILES: CC1(C)Cc2cccc(OCC(=O)Nc3cccc4cnccc34)c2O1]

2-((2,2-Dimethyl-2,3-dihydrobenzofuran-7-yl)oxy)-N-(isoquinolin-5-yl) acetamide (14) was prepared in accordance with the procedures described in Scheme 8 in 13% yield. ESIMS found for C₂₁H₂₀N₂O₃: m/z 349.4 (M+1).

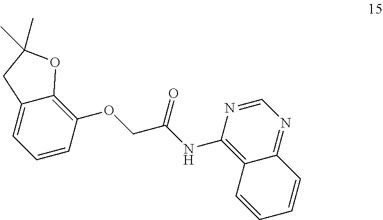 [SMILES: CC1(C)Cc2cccc(OCC(=O)Nc3ncnc4ccccc34)c2O1]

2-((2,2-Dimethyl-2,3-dihydrobenzofuran-7-yl)oxy)-N-(quinazolin-4-yl) acetamide (15) was prepared in accordance with the procedures described in Scheme 8 in 16% yield. ESIMS found for C₂₀H₁₉N₃O₃: m/z 350.5 (M+1).

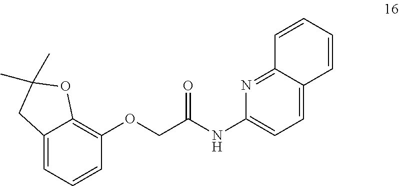 [SMILES: CC1(C)Cc2cccc(OCC(=O)Nc3ccc4ccccc4n3)c2O1]

2-((2,2-Dimethyl-2,3-dihydrobenzofuran-7-yl)oxy)-N-(quinolin-2-yl) acetamide (16) was prepared in accordance with the procedures described in Scheme 8 in 56% yield. ESIMS found for C₂₁H₂₀N₂O₃: m/z 349.4 (M+1).

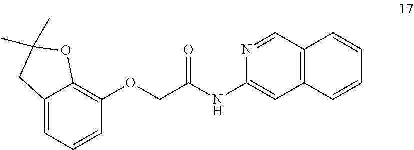 [SMILES: CC1(C)Cc2cccc(OCC(=O)Nc3cc4ccccc4cn3)c2O1]

2-((2,2-Dimethyl-2,3-dihydrobenzofuran-7-yl)oxy)-N-(isoquinolin-3-yl) acetamide (17) was prepared in accordance with the procedures described in Scheme 8 in 77% yield. ESIMS found for C₂₁H₂₀N₂O₃: m/z 347.2 (M+1).

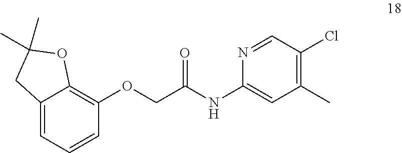 [SMILES: Cc1cc(NC(=O)COc2cccc3c2OC(C)(C)C3)ncc1Cl]

N-(5-Chloro-4-methylpyridin-2-yl)-2-((2,2-dimethyl-2,3-dihydrobenzofuran-7-yl)oxy)acetamide (18) was prepared in accordance with the procedures described in Scheme 8 in 10% yield. ESIMS found for C₁₈H₁₉ClN₂O₃: m/z 347.2 (M+1).

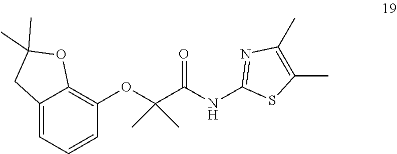

2-((2,2-Dimethyl-2,3-dihydrobenzofuran-7-yl)oxy)-N-(4,5-dimethylthiazol-2-yl)-2-methylpropanamide (19) was prepared in accordance with the procedures described in Scheme 8 from ethyl 2-bromo-2-methylpropanoate. ESIMS found for C₁₉H₂₄N₂O₃S: m/z 360.7 (M+1).

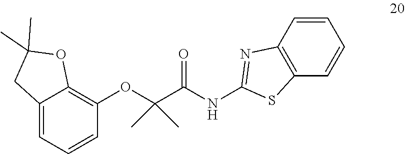 [SMILES: CC1(C)Cc2cccc(OC(C)(C)C(=O)Nc3nc4ccccc4s3)c2O1]

N-(Benzo[d]thiazol-2-yl)-2-((2,2-dimethyl-2,3-dihydrobenzofuran-7-yl)oxy)-2-methylpropanamide (20) was prepared in accordance with the procedures described in Scheme 8 from ethyl 2-bromo-2-methylpropanoate. ESIMS found for C₂₁H₂₂N₂O₃S: m/z 382.4 (M+1).

The preparation of 2-((2,2-dimethyl-2,3-dihydrobenzofuran-7-yl)oxy)-N-(4-(trifluoromethyl)thiazol-2-yl)acetamide (21) describing the coupling of an amine and a carboxylic acid to form an amide using Ghosez's reagent and pyridine is depicted below in Scheme 9.

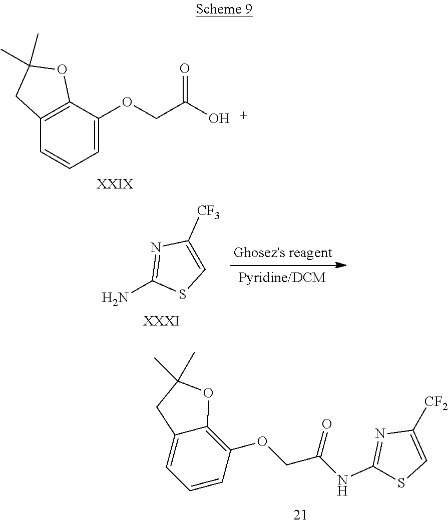

To a mixture of 2-((2,2-dimethyl-2,3-dihydrobenzofuran-7-yl)oxy)acetic acid (XXIX) (50 mg, 0.23 mmol) in anhydrous DCM (2 mL) cooled to 0° C. was added Ghosez's Reagent (1-chloro-N,N,2-trimethyl-1-propenylamine, 60 mg, 0.45 mmol). After 10 min, a solution of 4-(trifluoromethyl)thiazol-2-amine (XXXI) (37.8 mg, 0.225 mmol) in 0.2 mL pyridine and DCM (0.2 mL) was added. After 1 hour, the reaction was diluted with DCM (50 mL), then washed with brine (2×40 mL). The organic layer was dried over Na₂SO₄, filtered, and concentrated under reduced pressure. Flash chromatography (EtOAc:hexanes, gradient elution) afforded 16 mg (19%) of 2-((2,2-dimethyl-2,3-dihydrobenzofuran-7-yl)oxy)-N-(4-(trifluoromethyl)thiazol-2-yl)acetamide (21). ¹H NMR (400 MHz, CHLOROFORM-d) 6 ppm 1.53 (s, 6H), 3.05 (s, 2H), 4.81 (s, 2H), 6.75-6.85 (m, 2H), 6.90 (br d, J=6.36 Hz, 1H), 7.42 (s, 1H), 10.45 (br s, 1H); ESIMS found for C₁₆H₁₅F₃N₂O₃S: m/z 372.9 (M+1).

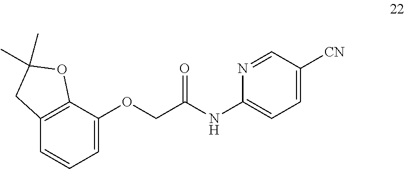 [SMILES: CC1(C)Cc2cccc(OCC(=O)Nc3ccc(C#N)cn3)c2O1]

N-(5-Cyanopyridin-2-yl)-2-((2,2-dimethyl-2,3-dihydrobenzofuran-7-yl)oxy) acetamide (22) was prepared in accordance with the procedures described in Scheme 9. 28% yield. ¹H NMR (400 MHz, CHLOROFORM-d) δ ppm 1.52 (br s, 6H), 3.06 (br s, 2H), 4.74 (br s, 2H), 6.74-6.86 (m, 2H), 6.90 (br d, J=6.24 Hz, 1H), 7.97 (br d, J=8.44 Hz, 1H), 8.43 (br d, J=8.44 Hz, 1H), 8.61 (br s, 1H), 9.44 (br s, 1H); ESIMS found for C₁₈H₁₇N₃O₃: m/z 324.3 (M+1).

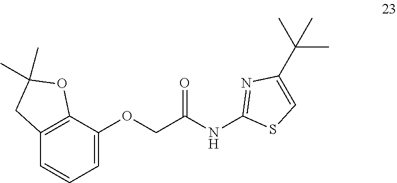 [SMILES: CC1(C)Cc2cccc(OCC(=O)Nc3nc(C(C)(C)C)cs3)c2O1]

N-(4-(tert-Butyl)thiazol-2-yl)-2-((2,2-dimethyl-2,3-dihydrobenzofuran-7-yl) oxy)acetamide (23) was prepared in accordance with the procedures described in Scheme 9. 43% yield. ¹H NMR (400 MHz, CHLOROFORM-d) δ ppm 1.31 (s, 9H), 1.55 (s, 6H), 3.05 (s, 2H), 4.77 (s, 2H), 6.56 (s, 1H), 6.72-6.87 (m, 2H), 6.89 (br d, J=6.97 Hz, 1H), 10.12 (br s, 1H); ESIMS found for C₁₉H₂₄N₂O₃S: m/z 361.1 (M+1).

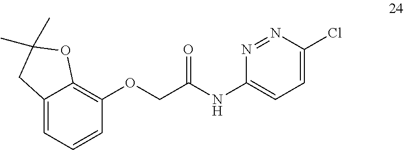 [SMILES: CC1(C)Cc2cccc(OCC(=O)Nc3ccc(Cl)nn3)c2O1]

N-(6-Chloropyridazin-3-yl)-2-((2,2-dimethyl-2,3-dihydrobenzofuran-7-yl) oxy)acetamide (24) was prepared in accordance with the procedures described in Scheme 9. 13% yield. ¹H NMR (400 MHz, CHLOROFORM-d) δ ppm 1.53 (s, 6H), 3.06 (s, 2H), 4.76 (s, 2H), 6.74-6.87 (m, 2H), 6.91 (dd, J=7.03, 1.16 Hz, 1H), 7.53 (d, J=9.29 Hz, 1H), 8.57 (d, J=9.29 Hz, 1H), 9.75 (br s, 1H); ESIMS found for C₁₆H₁₆ClN₃O₃: m/z 334.2 (M+1).

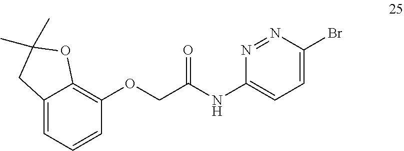 [SMILES: CC1(C)Cc2cccc(OCC(=O)Nc3ccc(Br)nn3)c2O1]

N-(6-Bromopyridazin-3-yl)-2-((2,2-dimethyl-2,3-dihydrobenzofuran-7-yl) oxy)acetamide (25) was prepared in accordance with the procedures described in Scheme 9. 49% yield. ¹H NMR (400 MHz, CHLOROFORM-d) δ ppm 1.53 (s, 6H), 3.06 (s, 2H), 4.75 (s, 2H), 6.75-6.85 (m, 2H), 6.90 (dd, J=7.03, 1.16 Hz, 1H), 7.65 (d, J=9.41 Hz, 1H), 8.47 (d, J=9.29 Hz, 1H), 9.77 (br s, 1H); ESIMS found for C₁₆H₁₆BrN₃O₃: m/z 378.2 (M+1).

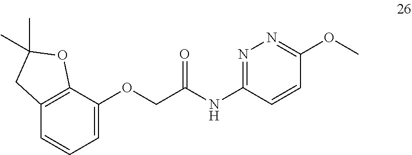

2-((2,2-Dimethyl-2,3-dihydrobenzofuran-7-yl)oxy)-N-(6-methoxypyridazin-3-yl)acetamide (26) was prepared in accordance with the procedures described in Scheme 9. 11% yield. ¹H NMR (400 MHz, CHLOROFORM-d) δ ppm 1.52 (s, 6H), 3.05 (s, 2H), 4.10 (s, 3H), 4.73 (s, 2H), 6.73-6.84 (m, 2H), 6.88 (d, J=6.97 Hz, 1H), 7.03 (d, J=9.54 Hz, 1H), 8.45 (d, J=9.54 Hz, 1H), 9.41 (br s, 1H); ESIMS found for C₁₇H₁₉N₃O₄: m/z 330.0 (M+1).

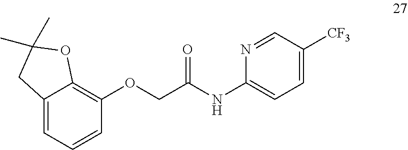 [SMILES: CC1(C)Cc2cccc(OCC(=O)Nc3ccc(C(F)(F)F)cn3)c2O1]

2-((2,2-Dimethyl-2,3-dihydrobenzofuran-7-yl)oxy)-N-(5-(trifluoromethyl) pyridin-2-yl)acetamide (27) was prepared in accordance with the procedures described in Scheme 9. 24% yield. ¹H NMR (400 MHz, CHLOROFORM-d) δ ppm 1.51 (s, 6H), 3.05 (s, 2H), 4.73 (s, 2H), 6.73-6.81 (m, 1H), 6.81-6.86 (m, 1H), 6.89 (dd, J=7.09, 0.98 Hz, 1H), 7.94 (dd, J=8.74, 2.26 Hz, 1H), 8.41 (d, J=8.80 Hz, 1H), 8.58 (broad s, 1H), 9.38 (br s, 1H); ESIMS found for C₁₈H₁₇F₃N₂O₃: m/z 367.3 (M+1).

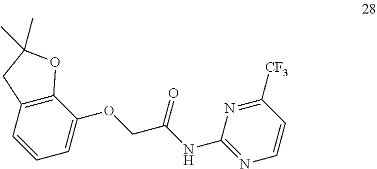 [SMILES: CC1(C)Cc2cccc(OCC(=O)Nc3nccc(C(F)(F)F)n3)c2O1]

2-((2,2-Dimethyl-2,3-dihydrobenzofuran-7-yl)oxy)-N-(4-(trifluoromethyl) pyrimidin-2-yl)acetamide (28) was prepared in accordance with the procedures described in Scheme 9. 29% yield. ¹H NMR (400 MHz, CHLOROFORM-d) δ ppm 1.52 (s, 6H), 3.05 (s, 2H), 4.79 (s, 2H), 6.74-6.81 (m, 1H), 6.83-6.87 (m, 1H), 6.90 (d, J=7.21 Hz, 1H), 7.38 (d, J=5.01 Hz, 1H), 8.96 (d, J=5.01 Hz, 1H), 9.61 (br s, 1H); ESIMS found for C₁₇H₁₆F₃N₃O₃: m/z 368.2 (M+1).

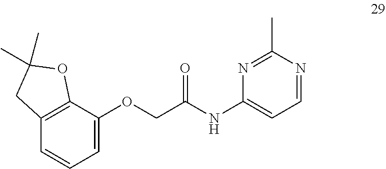 [SMILES: Cc1nccc(NC(=O)COc2cccc3c2OC(C)(C)C3)n1]

2-((2,2-Dimethyl-2,3-dihydrobenzofuran-7-yl)oxy)-N-(2-methylpyrimidin-4-yl)acetamide (29) was prepared in accordance with the procedures described in Scheme 9 in 14% yield. ESIMS found for C₁₇H₁₉N₃O₃: m/z 314.3 (M+1).

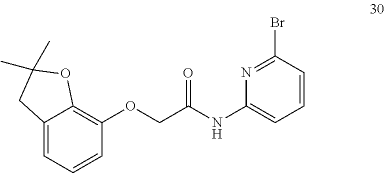 [SMILES: CC1(C)Cc2cccc(OCC(=O)Nc3cccc(Br)n3)c2O1]

N-(6-Bromopyridin-2-yl)-2-((2,2-dimethyl-2,3-dihydrobenzofuran-7-yl) oxy)acetamide (30) was prepared in accordance with the procedures described in Scheme 9. ESIMS found for C₁₇H₁₇BrN₂O₃: m/z 377.2.

The preparation of N-benzyl-4,5-dimethylthiazol-2-amine which describes the reductive amination of an amine and an aldehyde with NaBH₄ is depicted below in Scheme 10.

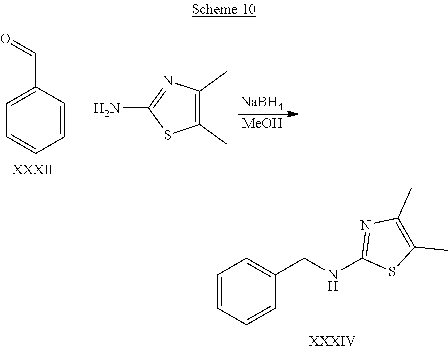

To a solution of benzaldehyde (XXXII) (1 g, 9.4 mmol) in MeOH (8 mL) was added 4,5-dimethylthiazol-2-amine (XXXIII) (1.2 g, 9.4 mmol). The mixture was stirred for 8 h at rt, then concentrated under reduced pressure. To the resulting crude residue was added MeOH (8 mL) followed by NaBH₄ (0.71 g, 18.9 mmol). The suspension was stirred for 30 min and the reaction mixture was quenched with water (100 mL), then EtOAc (150 mL) was added. The layers were separated and the organic phase was concentrated under reduced pressure. The resulting crude residue was purified by column chromatography (EtOAc:hexanes, gradient elution) to afford 0.8 g (39%) of N-benzyl-4,5-dimethylthiazol-2-amine (XXXIV). ESIMS found for C₁₂H₁₄N₂S: m/z 219.3 (M+1).

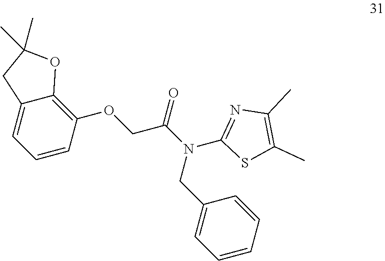

N-Benzyl-2-((2,2-dimethyl-2,3-dihydrobenzofuran-7-yl)oxy)-N-(4,5-dimethylthiazol-2-yl)acetamide (31) was prepared from 2-((2,2-dimethyl-2,3-dihydrobenzofuran-7-yl)oxy)acetic acid (XXIX) and N-benzyl-4,5-dimethylthiazol-2-amine (XXXIV) using the Ghosez's reagent coupling method in Scheme 9. 30% yield. ¹H NMR (400 MHz, CHLOROFORM-d) δ ppm 1.44 (s, 6H), 2.21 (s, 3H), 2.27 (s, 3H), 2.98 (s, 2H), 4.89 (br s, 2H), 5.53 (br s, 2H), 6.59-6.65 (m, 1H), 6.65-6.72 (m, 1H), 6.77 (d, J=7.21 Hz, 1H), 7.20-7.24 (m, 2H), 7.26-7.35 (m, 3H); ESIMS found for C₂₄H₂₆N₂O₃S: m/z 423.3 (M+1).

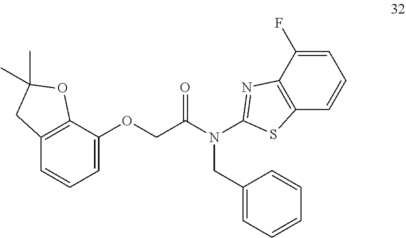 [SMILES: CC1(C)Cc2cccc(OCC(=O)N(Cc3ccccc3)c3nc4c(F)cccc4s3)c2O1]

N-Benzyl-2-((2,2-dimethyl-2,3-dihydrobenzofuran-7-yl)oxy)-N-(4-fluorobenzo[d]thiazol-2-yl)acetamide (32) was prepared in accordance with the procedures described in Schemes 10 and 9. ¹H NMR (400 MHz, CHLOROFORM-d) δ ppm 1.42 (s, 6H), 2.98 (s, 2H), 5.01 (s, 2H), 5.71 (s, 2H), 6.64-6.68 (m, 1H), 6.68-6.72 (m, 1H), 6.80 (dd, J=6.97, 0.98 Hz, 1H), 7.13 (ddd, J=10.55, 8.04, 0.86 Hz, 1H), 7.26-7.36 (m, 6H), 7.58 (d, J=7.95 Hz, 1H); ESIMS found for C₂₆H₂₃FN₂O₃S: m/z 463.0 (M+1).

The preparation of 4-methyl-N-(pyridin-2-ylmethyl)thiazol-2-amine which describes the reductive amination of an amine and an aldehyde with NaBH(OAc)₃ is depicted below in Scheme 11.

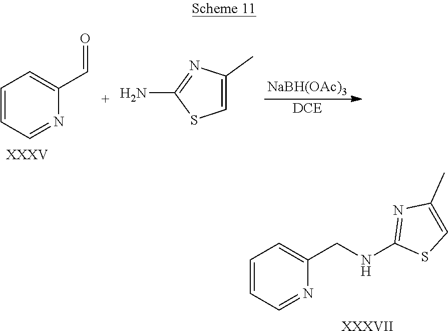

To a solution of pyridine-2-carboxaldehyde (XXXV) (0.5 g, 4.7 mmol) and 2-amino-4-methylthiazole (XXXVI) (0.59 g, 5.1 mmol) in DCE (5 mL) was added NaBH(OAc)₃ (2.0 g, 9.3 mmol). The reaction mixture was stirred for 10 h at rt and then diluted with 10% aq. NaHCO₃ (100 mL). EtOAc (100 mL) was then added, and the layers were separated. The organic phase was dried over MgSO4, filtered and concentrated, and the crude residue was purified by column chromatography (EtOAc:hexanes , gradient elution) to afford 0.24 g (25%) of 4-methyl-N-(pyridin-2-ylmethyl)thiazol-2-amine (XXXVII). ESIMS found for C₁₀H₁₁N₃S: m/z 206.3 (M+1).

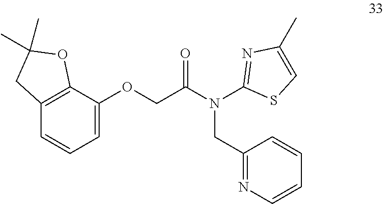 [SMILES: Cc1csc(N(Cc2ccccn2)C(=O)COc2cccc3c2OC(C)(C)C3)n1]

2-((2,2-Dimethyl-2,3-dihydrobenzofuran-7-yl)oxy)-N-(4-methylthiazol-2-yl)-N-(pyridin-2-ylmethyl)acetamide (33) was prepared in accordance with the amide coupling procedure described in Scheme 9 from 2-((2,2-dimethyl-2,3-dihydrobenzofuran-7-yl)oxy)acetic acid and 4-methyl-N-(pyridin-2-ylmethyl)thiazol-2-amine. 6.5% yield after purification by C18 reverse phase chromatography. ¹H NMR (400 MHz, CHLOROFORM-d) δ ppm 1.45 (s, 6H), 2.30 (s, 3H), 2.99 (s, 2H), 5.21 (s, 2H), 5.57 (br s, 2H), 6.55 (br s, 1H), 6.66-6.73 (m, 1H), 6.74-6.83 (m, 2H), 7.19 (br dd, J=6.66, 5.20 Hz, 1H), 7.34 (br d, J=7.46 Hz, 1H), 7.58-7.68 (m, 1H), 8.54 (br d, J=3.42 Hz, 1H); ESIMS found for C₂₂H₂₃N₃O₃S: m/z 410.4 (M+1).

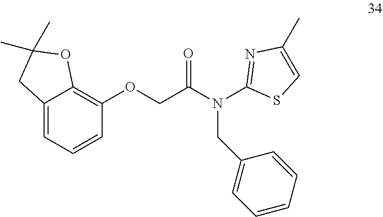

2-((2,2-Dimethyl-2,3-dihydrobenzofuran-7-yl)oxy)-N-(4-methylthiazol-2-yl)-N-(pyridin-3-ylmethyl)acetamide (34) was prepared in accordance with the procedures described in Schemes 11 and 9. ¹H NMR (400 MHz, CHLOROFORM-d) δ ppm 1.43 (s, 6H), 2.32 (s, 3H), 2.98 (s, 2H), 4.96 (br s, 2H), 5.60 (br s, 2H), 6.62 (br s, 1H), 6.70-6.75 (m, 2H), 6.77-6.83 (m, 1H), 7.23 (dd, J=7.83, 4.77 Hz, 1H), 7.58 (br d, J=6.48 Hz, 1H), 8.52 (br d, J=3.55 Hz, 1H), 8.58 (br s, 1H); ESIMS found for C₂₂H₂₃N₃O₃S: m/z 410.4 (M+1).

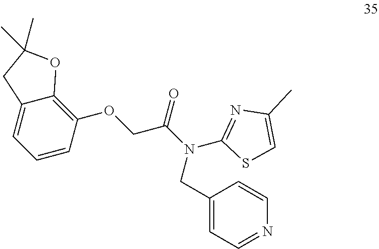

2-((2,2-Dimethyl-2,3-dihydrobenzofuran-7-yl)oxy)-N-(4-methylthiazol-2-yl)-N-(pyridin-4-ylmethyl)acetamide (35) was prepared in accordance with the procedures described in Schemes 11 and 9. ¹H NMR (400 MHz, DMSO-d6) δ ppm 1.31 (s, 6H), 2.21 (s, 3H), 2.95 (s, 2H), 5.01 (s, 2H), 5.48 (br s, 2H), 6.62-6.68 (m, 2H), 6.79 (dd, J=5.87, 2.69 Hz, 1H), 6.92 (s, 1H), 7.21 (d, J=5.87 Hz, 2H), 8.52 (d, J=5.62 Hz, 2H); ESIMS found for C₂₂H₂₃N₃O₃S: m/z 410.3 (M+1).

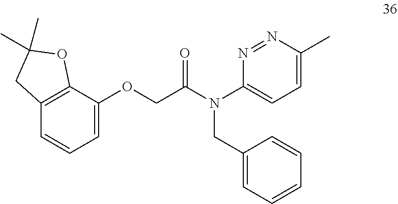

N-Benzyl-2-((2,2-dimethyl-2,3-dihydrobenzofuran-7-yl)oxy)-N-(6-methylpyridazin-3-yl)acetamide (36) was prepared in accordance with the procedures described in Schemes 11 and 9. ¹H NMR (400 MHz, CHLOROFORM-d) δ ppm 1.43 (s, 6H), 2.67 (s, 3H), 2.97 (s, 2H), 4.92 (s, 2H), 5.21 (br s, 2H), 6.63-6.67 (m, 1H), 6.67-6.73 (m, 1H), 6.74-6.79 (m, 1H), 7.20-7.26 (m, 7H); ESIMS found for C₂₄H₂₅N₃O₃: m/z 404.3 (M+1).

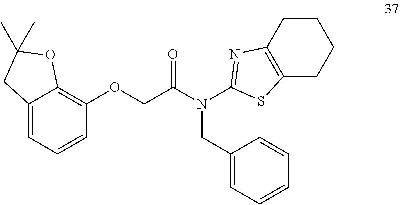 [SMILES: CC1(C)Cc2cccc(OCC(=O)N(Cc3ccccc3)c3nc4c(s3)CCCC4)c2O1]

N-Benzyl-2-((2,2-dimethyl-2,3-dihydrobenzofuran-7-yl)oxy)-N-(4,5,6,7-tetrahydrobenzo[d]thiazol-2-yl)acetamide (37) was prepared in accordance with the procedures described in Schemes 11 and 9. ¹H NMR (400 MHz, CHLOROFORM-d) δ ppm 1.43 (s, 6H), 1.78-1.87 (m, 4H), 2.63 (br s, 2H), 2.68 (br s, 2H), 2.97 (s, 2H), 4.90 (br s, 2H), 5.53 (br s, 2H), 6.59-6.64 (m, 1H), 6.64-6.71 (m, 1H), 6.77 (d, J=7.21 Hz, 1H), 7.20-7.27 (m, 3H), 7.27-7.34 (m, 2H); ESIMS found for C₂₆H₂₈N₂O₃S: m/z 449.3 (M+1).

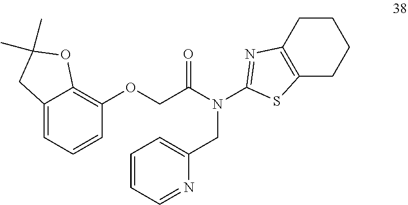 [SMILES: CC1(C)Cc2cccc(OCC(=O)N(Cc3ccccn3)c3nc4c(s3)CCCC4)c2O1]

2-((2,2-Dimethyl-2,3-dihydrobenzofuran-7-yl)oxy)-N-(pyridin-2-ylmethyl)-N-(4,5,6,7-tetrahydrobenzo[d]thiazol-2-yl)acetamide (38) was prepared in accordance with the procedures described in Schemes 11 and 9. ¹H NMR (400 MHz, CHLOROFORM-d) δ ppm 1.46 (s, 6H), 1.77-1.87 (m, 4H), 2.62 (br s, 2H), 2.67 (br s, 2H), 3.00 (s, 2H), 5.17 (s, 2H), 5.55 (br s, 2H), 6.66-6.74 (m, 1H), 6.75-6.81 (m, 2H), 7.14-7.24 (m, 1H), 7.34 (d, J=7.82 Hz, 1H), 7.63 (td, J=7.70, 1.83 Hz, 1H), 8.50-8.57 (m, 1H); ESIMS found for C₂₅H₂₇N₃O₃S: m/z 450.2 (M+1).

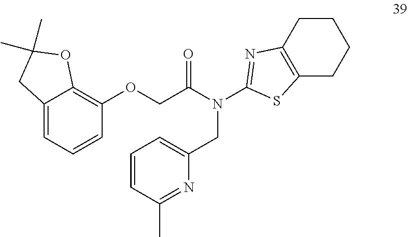 [SMILES: Cc1cccc(CN(C(=O)COc2cccc3c2OC(C)(C)C3)c2nc3c(s2)CCCC3)n1]

2-((2,2-Dimethyl-2,3-dihydrobenzofuran-7-yl)oxy)-N-((6-methylpyridin-2-yl)methyl)-N-(4,5,6,7-tetrahydrobenzo[d]thiazol-2-yl)acetamide (39) was prepared in accordance with the procedures described in Schemes 11 and 9. ¹H NMR (400 MHz, CHLOROFORM-d) δ ppm 1.47 (s, 6H), 1.82 (br s, 4H), 2.53 (s, 3H), 2.62 (br s, 2H), 2.67 (br s, 2H), 3.00 (s, 2H), 5.21 (br s, 2H), 5.48 (br s, 2H), 6.66-6.73 (m, 1H), 6.75-6.82 (m, 2H), 7.04 (d, J=7.70 Hz, 1H), 7.12 (d, J=7.58 Hz, 1H), 7.51 (t, J=7.70 Hz, 1H); ESIMS found for C₂₆H₂₉N₃O₃S: m/z 464.2 (M+1).

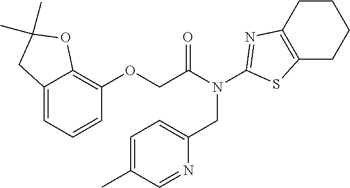 [SMILES: Cc1ccc(CN(C(=O)COc2cccc3c2OC(C)(C)C3)c2nc3c(s2)CCCC3)nc1]

2-((2,2-Dimethyl-2,3-dihydrobenzofuran-7-yl)oxy)-N-((5-methylpyridin-2-yl)methyl)-N-(4,5,6,7-tetrahydrobenzo[d]thiazol-2-yl)acetamide (40) was prepared in accordance with the procedures described in Schemes 11 and 9. ¹H NMR (400 MHz, CHLOROFORM-d) δ ppm 1.46 (s, 6H), 1.82 (br s, 4H), 2.31 (s, 3H), 2.62 (br s, 2H), 2.67 (br s, 2H), 3.00 (s, 2H), 5.17 (br s, 2H), 5.50 (br s, 2H), 6.66-6.74 (m, 1H), 6.75-6.81 (m, 2H), 7.23 (br d, J=7.95 Hz, 1H), 7.43 (br d, J=7.58 Hz, 1H), 8.36 (s, 1H); ESIMS found for C₂₆H₂₉N₃O₃S: m/z 464.1 (M+1).

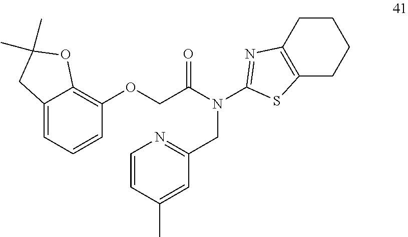 [SMILES: Cc1ccnc(CN(C(=O)COc2cccc3c2OC(C)(C)C3)c2nc3c(s2)CCCC3)c1]

2-((2,2-Dimethyl-2,3-dihydrobenzofuran-7-yl)oxy)-N-((4-methylpyridin-2-yl)methyl)-N-(4,5,6,7-tetrahydrobenzo[d]thiazol-2-yl)acetamide (41) was prepared in accordance with the procedures described in Schemes 11 and 9. ¹H NMR (400 MHz, CHLOROFORM-d) δ ppm 1.46 (s, 6H), 1.83 (br s, 4H), 2.31 (s, 3H), 2.63 (br s, 2H), 2.68 (br s, 2H), 2.99 (s, 2H), 5.13 (s, 2H), 5.52 (br s, 2H), 6.66-6.74 (m, 1H), 6.76 (br s, 2H), 7.00 (br d, J=4.28 Hz, 1H), 7.12 (br s, 1H), 8.39 (br d, J=4.65 Hz, 1H); ESIMS found for C₂₆H₂₉N₃O₃S: m/z 464.4 (M+1).

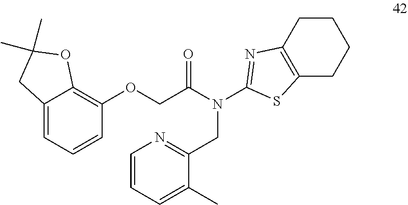 [SMILES: Cc1cccnc1CN(C(=O)COc1cccc2c1OC(C)(C)C2)c1nc2c(s1)CCCC2]

2-((2,2-Dimethyl-2,3-dihydrobenzofuran-7-yl)oxy)-N-((3-methylpyridin-2-yl)methyl)-N-(4,5,6,7-tetrahydrobenzo[d]thiazol-2-yl)acetamide (42) was prepared in accordance with the procedures described in Schemes 11 and 9. ¹H NMR (400 MHz, CHLOROFORM-d) δ ppm 1.48 (s, 6H), 1.79 (br s, 4H), 2.49 (s, 3H), 2.56 (br s, 2H), 2.64 (br s, 2H), 3.01 (s, 2H), 5.17 (br s, 2H), 5.52 (br s, 2H), 6.70-6.76 (m, 1H), 6.76-6.82 (m, 1H), 6.96 (br s, 1H), 7.08 (dd, J=7.40, 4.83 Hz, 1H), 7.43 (br d, J=7.34 Hz, 1H), 8.34 (br d, J=3.55 Hz, 1H); ESIMS found for C₂₆H₂₉N₃O₃S: m/z 464.3 (M+1).

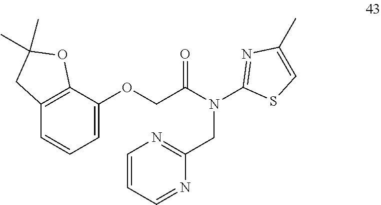

2-((2,2-Dimethyl-2,3-dihydrobenzofuran-7-yl)oxy)-N-(4-methylthiazol-2-yl)-N-(pyrimidin-2-ylmethyl)acetamide (43) was prepared in accordance with the procedures described in Schemes 11 and 9. ¹H NMR (400 MHz, CHLOROFORM-d) δ ppm 1.45 (s, 6H), 3.00 (s, 2H), 3.50 (s, 2H), 5.05 (br s, 2H), 5.75 (broad s, 2H), 6.53 (br s, 1H), 6.69-6.75 (m, 1H), 6.77-6.82 (m, 1H), 6.85 (d, J=7.95 Hz, 1H), 7.20 (t, J=4.89 Hz, 1H), 8.69 (d, J=4.89 Hz, 2H); ESIMS found for C₂₁H₂₂N₄O₃S: m/z 411.3 (M+1).

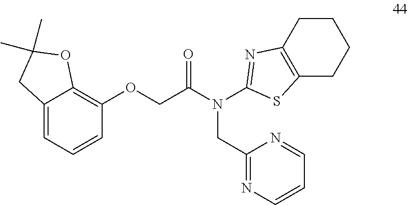 [SMILES: CC1(C)Cc2cccc(OCC(=O)N(Cc3ncccn3)c3nc4c(s3)CCCC4)c2O1]

2-((2,2-Dimethyl-2,3-dihydrobenzofuran-7-yl)oxy)-N-(pyrimidin-2-ylmethyl)-N-(4,5,6,7-tetrahydrobenzo[d]thiazol-2-yl)acetamide (44) was prepared in accordance with the procedures described in Schemes 11 and 9. ¹H NMR (400 MHz, CHLOROFORM-d) δ ppm 8.69 (d, J=4 Hz, 2H), 7.18 (d, J=4 Hz, 1H), 6.85-6.67 (m, 3H), 5.72 (broad s, 2H), 5.02 (broad s, 2H), 2.99 (s, 2H), 2.70-2.60 (m, 2H), 2.60-2.50 (m, 2H), 1.84-1.74 (m, 4H), 1.45 (s, 6H); ESIMS found for C₂₄H₂₆N₄O₃S: m/z 451.3 (M+1).

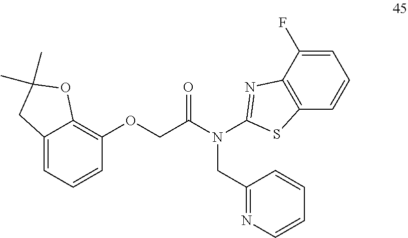 [SMILES: CC1(C)Cc2cccc(OCC(=O)N(Cc3ccccn3)c3nc4c(F)cccc4s3)c2O1]

2-((2,2-Dimethyl-2,3-dihydrobenzofuran-7-yl)oxy)-N-(4-fluorobenzo[d]thiazol-2-yl)-N-(pyridin-2-ylmethyl)acetamide (45) was prepared in accordance with the procedures described in Schemes 11 and 9. ¹H NMR (400 MHz, CHLOROFORM-d) δ ppm 1.47 (s, 6H), 3.02 (s, 2H), 5.41 (s, 2H), 5.69 (s, 2H), 6.70-6.79 (m, 1H), 6.79-6.85 (m, 1H), 6.88 (d, J=7.95 Hz, 1H), 7.11 (dd, J=10.15, 8.44 Hz, 1H), 7.17-7.25 (m, 2H), 7.50-7.59 (m, 2H), 7.67 (td, J=7.70, 1.71 Hz, 1H), 8.54 (d, J=4.28 Hz, 1H); ESIMS found for C₂₅H₂₂FN₃O₃S: m/z 464.2 (M+1).

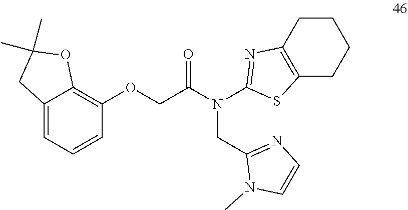 [SMILES: Cn1ccnc1CN(C(=O)COc1cccc2c1OC(C)(C)C2)c1nc2c(s1)CCCC2]

2-((2,2-Dimethyl-2,3-dihydrobenzofuran-7-yl)oxy)-N-((1-methyl-1H-imidazol-2-yl)methyl)-N-(4,5,6,7-tetrahydrobenzo[d]thiazol-2-yl)acetamide (46) was prepared in accordance with the procedures described in Schemes 11 and 9. ¹H NMR (400 MHz, CHLOROFORM-d) δ ppm 1.47 (s, 6H), 1.77-1.87 (m, 4H), 2.58-2.70 (m, 4H), 3.00 (s, 2H), 3.80 (br s, 3H), 5.12 (br s, 2H), 5.45 (br s, 2H), 6.67-6.74 (m, 1H), 6.75-6.82 (m, 2H), 6.86 (br s, 1H), 6.94 (s, 1H); ESIMS found for C₂₄H₂₈N₄O₃S: m/z 453.2 (M+1).

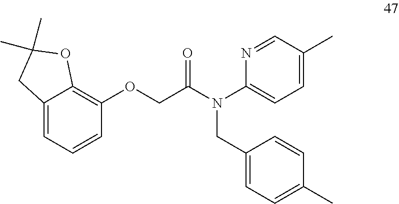

2-((2,2-Dimethyl-2,3-dihydrobenzofuran-7-yl)oxy)-N-(4-methylbenzyl)-N-(5-methylpyridin-2-yl)acetamide (47) was prepared in accordance with the procedures described in Schemes 11 and 9. ¹H NMR (400 MHz, CHLOROFORM-d) δ ppm 8.26 (d, J=2 Hz, 1H), 7.42 (dd, J=8, 2 Hz, 1H), 7.09 (d, J=8 z, 2H), 7.04 (d, J=8 Hz, 2H), 6.96-0.90 (broad s, 1H), 6.77-6.65 (m, 3H), 5.01 (s, 2H), 4.78 (s, 2H), 2.97 (s, 2H), 2.31 (s, 3H), 2.29 (s, 3H), 1.43 (s, 6H); ESIMS found for C₂₆H₂₈N₂O₃: m/z 417.4 (M+1).

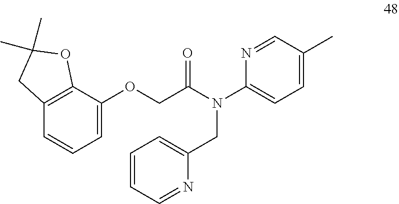

2-((2,2-Dimethyl-2,3-dihydrobenzofuran-7-yl)oxy)-N-(5-methylpyridin-2-yl)-N-(pyridin-2-ylmethyl)acetamide (48) was prepared in accordance with the NaBH(OAc)₃ reductive amination procedure described in Scheme 11 followed by Step 3 in Scheme 8 from 2-((2,2-dimethyl-2,3-dihydrobenzofuran-7-yl)oxy)acetic acid and 5-methyl-N-(pyridin-2-ylmethyl)pyridin-2-amine. ¹H NMR (400 MHz, CHLOROFORM-d) δ ppm 1.44 (s, 6H), 2.32 (s, 3H), 2.98 (s, 2H), 4.93 (s, 2H), 5.17 (s, 2H), 6.66-6.71 (m, 2H), 6.72-6.77 (m, 1H), 7.14 (dd, J=7.27, 5.07 Hz, 1H), 7.21-7.27 (m, 1H), 7.41 (d, J=7.82 Hz, 1H), 7.49 (dd, J=8.13, 2.38 Hz, 1H), 7.61 (td, J=7.67, 1.77 Hz, 1H), 8.23 (d, J=1.83 Hz, 1H), 8.49 (d, J=4.77 Hz, 1H); ESIMS found for C₂₄H₂₅N₃O₃: m/z 404.4 (M+1).

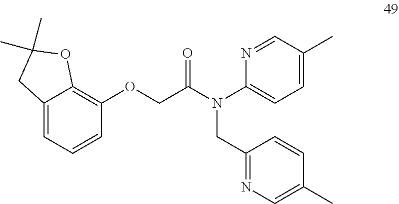

2-((2,2-Dimethyl-2,3-dihydrobenzofuran-7-yl)oxy)-N-(5-methylpyridin-2-yl)-N-((5-methylpyridin-2-yl)methyl)acetamide (49) was prepared in accordance with the procedures described in Schemes 11 and 8. ¹H NMR (400 MHz, CHLOROFORM-d) δ ppm 1.43 (s, 6H), 2.27 (s, 3H), 2.30 (s, 3H), 2.96 (s, 2H), 4.91 (s, 2H), 5.13 (s, 2H), 6.64-6.69 (m, 2H), 6.71-6.77 (m, 1H), 7.22 (br d, J=6.60 Hz, 1H), 7.29 (d, J=7.95 Hz, 1H), 7.40 (dd, J=7.95, 1.83 Hz, 1H), 7.47 (dd, J=8.19, 1.96 Hz, 1H), 8.22 (d, J=2.20 Hz, 1H), 8.29 (s, 1H); ESIMS found for C₂₅H₂₇N₃O₃: m/z 418.4 (M+1).

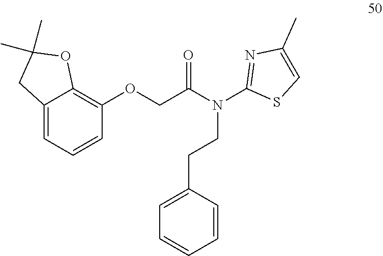 [SMILES: Cc1csc(N(CCc2ccccc2)C(=O)COc2cccc3c2OC(C)(C)C3)n1]

2-((2,2-Dimethyl-2,3-dihydrobenzofuran-7-yl)oxy)-N-(4-methylthiazol-2-yl)-N-phenethylacetamide (50) was prepared in accordance with the procedures described in Schemes 11 and 8. ¹H NMR (400 MHz, CHLOROFORM-d) δ ppm 1.44 (s, 6H), 2.40 (s, 3H), 2.98 (s, 2H), 3.12 (br t, J=6.48 Hz, 2H), 4.36 (br s, 2H), 4.75 (br s, 2H), 6.55-6.65 (m, 2H), 6.70 (t, J=7.70 Hz, 1H), 6.78 (dd, J=7.34, 0.98 Hz, 1H), 7.20-7.26 (m, 3H), 7.27-7.34 (m, 2H); ESIMS found for C₂₄H₂₆N₂O₃S: m/z 423.4 (M+1).

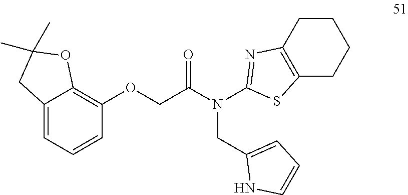 [SMILES: CC1(C)Cc2cccc(OCC(=O)N(Cc3ccc[nH]3)c3nc4c(s3)CCCC4)c2O1]

N-((1H-Pyrrol-2-yl)methyl)-2-((2,2-dimethyl-2,3-dihydrobenzofuran-7-yl)oxy)-N-(4,5,6,7-tetrahydrobenzo[d]thiazol-2-yl)acetamide (51) was prepared in accordance with the procedures described in Schemes 11 and 8. ¹H NMR (400 MHz, CHLOROFORM-d) δ ppm 1.47 (s, 6H), 1.80-1.90 (m, 4H), 2.66-2.72 (m, 2H), 2.74 (br s, 2H), 3.01 (s, 2H), 5.15 (br s, 2H), 5.24 (br s, 2H), 6.09 (q, J=2.89 Hz, 1H), 6.21 (br s, 1H), 6.68-6.78 (m, 2H), 6.80 (br d, J=7.70 Hz, 1H), 9.64 (br s, 1H); ESIMS found for C₂₄H₂₇N₃O₃S: m/z 438.2 (M+1).

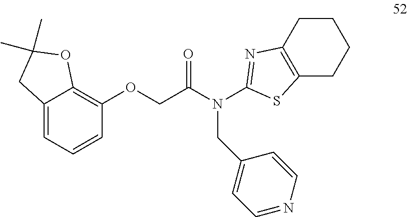 [SMILES: CC1(C)Cc2cccc(OCC(=O)N(Cc3ccncc3)c3nc4c(s3)CCCC4)c2O1]

2-((2,2-Dimethyl-2,3-dihydrobenzofuran-7-yl)oxy)-N-(pyridin-4-ylmethyl)-N-(4,5,6,7-tetrahydrobenzo[d]thiazol-2-yl)acetamide (52) was prepared in accordance with the procedures described in Schemes 11 and 8. ¹H NMR (400 MHz, CHLOROFORM-d) δ ppm 1.42 (s, 6H), 1.82 (br t, J=2.81 Hz, 4H), 2.59 (br s, 2H), 2.69 (br s, 2H), 2.97 (s, 2H), 4.89 (br s, 2H), 5.57 (br s, 2H), 6.70 (d, J=4.52 Hz, 2H), 6.77-6.82 (m, 1H), 7.13 (br s, 2H), 8.52 (br d, J=4.04 Hz, 2H); ESIMS found for C₂₅H₂₇N₃O₃S: m/z 450.0 (M+1).

Preparation of N-methyl-4,5,6,7-tetrahydrobenzo[d]thiazol-2-amine from 2-chlorocyclohexanone and N-methylthiourea is depicted below in Scheme 12.

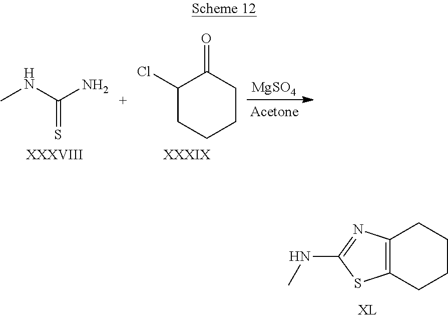

To a suspension of methylthiourea (XXXVIII) (10.0 g, 111 mmol), MgSO4 (6.7 g, 55 mmol) in acetone (10 mL) at reflux was added a solution of 2-chlorocyclohexanone (XXXIX) (10.2 g, 111 mmol) and acetone (10 mL) over 15 min. The reaction mixture was heated at reflux for 1 h, and the reaction mixture was cooled to rt. The solution was poured into brine (80 mL), and basified with a solution of concentrated NH₄OH. EtOAc (200 mL) was added and layers were partition and separated. The organic phase was dried over Na₂SO₄, filtered and concentrated under reduced pressure. The resulting crude residue was purified by column chromatography (EtOAc:hexanes, gradient elution) to afford 8.0 g (43%) of N-methyl-4,5,6,7-tetrahydrobenzo[d]thiazol-2-amine (XL). ESIMS found for C₈H₁₂N₂S: m/z 169.2 (M+1).

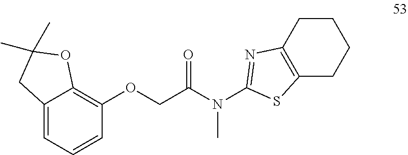 [SMILES: CN(C(=O)COc1cccc2c1OC(C)(C)C2)c1nc2c(s1)CCCC2]

2-((2,2-Dimethyl-2,3-dihydrobenzofuran-7-yl)oxy)-N-methyl-N-(4,5,6,7-tetrahydrobenzo[d]thiazol-2-yl)acetamide (53) was prepared from 2-((2,2-dimethyl-2,3-dihydrobenzofuran-7-yl)oxy)acetic acid and N-methyl-4,5,6,7-tetrahydrobenzo[d]thiazol-2-amine using the HATU amide coupling method used in Scheme 8. 23% yield. ¹H NMR (400 MHz, CHLOROFORM-d) δ ppm 1.47 (s, 6H), 1.78-1.91 (m, 4H), 2.63-2.73 (m, 4H), 3.00 (s, 2H), 3.70 (br s, 3H), 5.03 (s, 2H), 6.69-6.75 (m, 1H), 6.76-6.84 (m, 2H); ESIMS found for C₂₀H₂₄N₂O₃S: m/z 373.3.

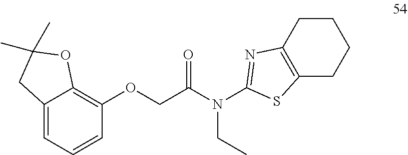 [SMILES: CCN(C(=O)COc1cccc2c1OC(C)(C)C2)c1nc2c(s1)CCCC2]

2-((2,2-Dimethyl-2,3-dihydrobenzofuran-7-yl)oxy)-N-ethyl-N-(4,5,6,7-tetrahydrobenzo[d]thiazol-2-yl)acetamide (54) was prepared in accordance with the procedures described in Schemes 12 and 8. ¹H NMR (400 MHz, CHLOROFORM-d) δ ppm 1.30-1.42 (m, 3H), 1.47 (s, 6H), 1.79-1.90 (m, 4H), 2.64-2.73 (m, 4H), 3.00 (s, 2H), 4.21 (br s, 2H), 5.02 (br s, 2H), 6.70-6.75 (m, 1H), 6.76-6.84 (m, 2H); ESIMS found for C₂₁H₂₆N₂O₃S: m/z 387.2 (M+1).

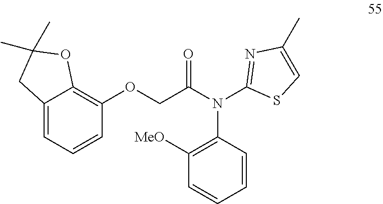

2-((2,2-Dimethyl-2,3-dihydrobenzofuran-7-yl)oxy)-N-(2-methoxyphenyl)-N-(4-methylthiazol-2-yl)acetamide (55) was prepared in accordance with the procedures described in Schemes 12 and 8. ¹H NMR (400 MHz, CHLOROFORM-d) δ ppm 1.44 (s, 6H), 2.19 (d, J=0.73 Hz, 3H), 2.97 (s, 2H), 3.79 (s, 3H), 4.45-4.70 (m, 2H), 6.54 (d, J=0.98 Hz, 1H), 6.67-6.71 (m, 2H), 6.73-6.78 (m, 1H), 7.04-7.13 (m, 2 H), 7.32 (dd, J=7.70, 1.71 Hz, 1H), 7.43-7.50 (m, 1H); ESIMS found for C₂₃H₂₄N₂O₄S: m/z 425.3 (M+1).

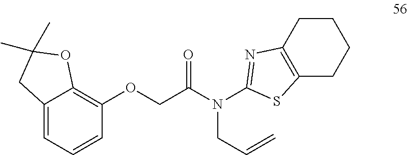 [SMILES: C=CCN(C(=O)COc1cccc2c1OC(C)(C)C2)c1nc2c(s1)CCCC2]

N-Allyl-2-((2,2-dimethyl-2,3-dihydrobenzofuran-7-yl)oxy)-N-(4,5,6,7-tetrahydrobenzo[d]thiazol-2-yl)acetamide (56) was prepared in accordance with the procedures described in Schemes 12 and 8. ¹H NMR (400 MHz, CHLOROFORM-d) δ ppm 1.48 (s, 6H), 1.79-1.90 (m, 4H), 2.63-2.73 (m, 4H), 3.01 (s, 2H), 4.88 (br s, 2H), 5.02 (s, 2H), 5.18-5.23 (m, 1H), 5.24 (s, 1H), 5.31 (s, 1H), 5.98 (br s, 1H), 6.69-6.78 (m, 2H), 6.78-6.84 (m, 1H); ESIMS found for C₂₂H₂₆N₂O₃S: m/z 399.4 (M+1).

The synthesis of 2-((2,2-dimethyl-2,3-dihydrobenzofuran-7-yl)oxy)-N-propyl-N-(4,5,6,7-tetrahydrobenzo[d]thiazol-2-yl)acetamide (62) is depicted in Scheme 13.

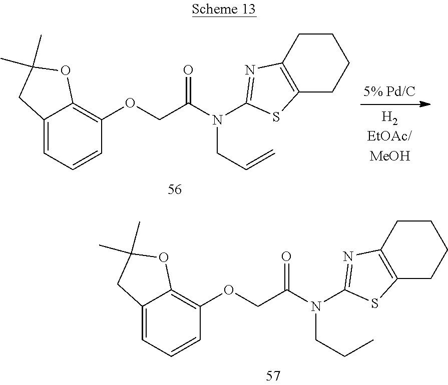 [SMILES: C=CCN(C(=O)COc1cccc2c1OC(C)(C)C2)c1nc2c(s1)CCCC2 ; CCCN(C(=O)COc1cccc2c1OC(C)(C)C2)c1nc2c(s1)CCCC2]

A solution of N-allyl-2-((2,2-dimethyl-2,3-dihydrobenzofuran-7-yl)oxy)-N-(4,5,6,7-tetrahydrobenzo[d]thiazol-2-yl)acetamide (56) (50 mg, 0.13 mmol) in EtOAc (1 mL) and MeOH (1 mL) was flushed with nitrogen. 5% Pd—C (10 mg) was added and the flask was flushed sequentially with nitrogen and hydrogen, and the flask was place under a balloon of hydrogen. After 16 h, the mixture was filtered through a pad of Celite®, the cake was washed with EtOAc (30 mL), and the filtrate was concentrated. Flash chromatography (EtOAc:hexanes, gradient elution from 0% EtOAc to 100% EtOAc) afforded 21 mg (42%) of compound 57. ¹H NMR (400 MHz, CHLOROFORM-d) δ ppm 0.98 (br t, J=7.03 Hz, 3H), 1.48 (s, 6H), 1.73-1.92 (m, 6H), 2.63-2.74 (m, 4H), 3.01 (s, 2H), 4.07 (br s, 2H), 5.02 (br s, 2H), 6.70-6.76 (m, 1H), 6.76-6.86 (m, 2H); ESIMS found for C₂₂H₂₈N₂O₃S: m/z 401.4 (M+1).

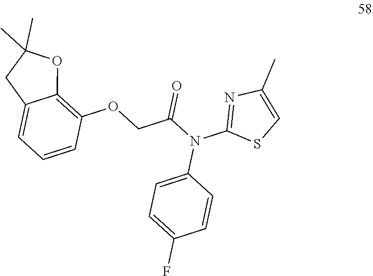 [SMILES: Cc1csc(N(C(=O)COc2cccc3c2OC(C)(C)C3)c2ccc(F)cc2)n1]

2-((2,2-Dimethyl-2,3-dihydrobenzofuran-7-yl)oxy)-N-(4-fluorophenyl)-N-(4-methylthiazol-2-yl)acetamide (58) was prepared from N-(4-fluorophenyl)-4-methylthiazol-2-amine in accordance with the procedures described in Schemes 12 and 9. ¹H NMR (400 MHz, CHLOROFORM-d) δ ppm 1.43 (s, 6H), 2.22 (s, 3H), 2.97 (s, 2H), 4.64 (s, 2H), 6.59 (s, 1H), 6.71 (d, J=4.65 Hz, 2H), 6.76-6.81 (m, 1H), 7.18-7.24 (m, 2H), 7.31-7.37 (m, 2H); ESIMS found for C₂₂H₂₁FN₂O₃S: m/z 413.4 (M+1).

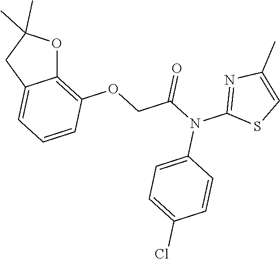 [SMILES: Cc1csc(N(C(=O)COc2cccc3c2OC(C)(C)C3)c2ccc(Cl)cc2)n1]

N-(4-Chlorophenyl)-2-((2,2-dimethyl-2,3-dihydrobenzofuran-7-yl) oxy)-N-(4-methylthiazol-2-yl)acetamide (59) was prepared in accordance with the procedures described in Schemes 12 and 9. ¹H NMR (400 MHz, CHLOROFORM-d) δ ppm 1.43 (s, 6H), 2.22 (s, 3H), 2.97 (s, 2H), 4.65 (s, 2H), 6.59 (s, 1H), 6.71 (d, J=4.52 Hz, 2H), 6.76-6.81 (m, 1H), 7.29 (d, J=8.56 Hz, 2H), 7.50 (d, J=8.56 Hz, 2H); ESIMS found for C₂₂H₂₁ClN₂O₃S: m/z 429.1 (M+1).

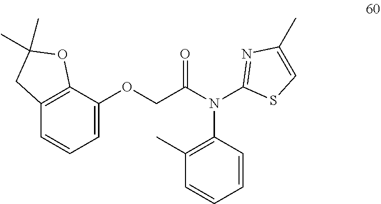

2-((2,2-Dimethyl-2,3-dihydrobenzofuran-7-yl)oxy)-N-(4-methylthiazol-2-yl)-N-(o-tolyl)acetamide (60) was prepared in accordance with the procedures described in Schemes 12 and 9. ¹H NMR (400 MHz, CHLOROFORM-d) δ ppm 1.42 (s, 3H), 1.44 (s, 3H), 2.15 (s, 3H), 2.20 (s, 3H), 2.97 (s, 2H), 4.39 (br d, J=15.77 Hz, 1H), 4.68 (br d, J=16.26 Hz, 1H), 6.56 (d, J=0.98 Hz, 1H), 6.70 (d, J=4.65 Hz, 2H), 6.75-6.80 (m, 1H), 7.26-7.30 (m, 1H), 7.32-7.45 (m, 3H); ESIMS found for C₂₃H₂₄N₂O₃S: m/z 409.4 (M+1).

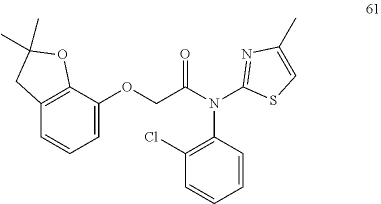

N-(2-Chlorophenyl)-2-((2,2-dimethyl-2,3-dihydrobenzofuran-7-yl)oxy)-N-(4-methylthiazol-2-yl)acetamide (61) was prepared in accordance with the procedures described in Schemes 12 and 9. ¹H NMR (400 MHz, CHLOROFORM-d) δ ppm 1.45 (s, 6H), 2.22 (s, 3H), 2.99 (s, 2H), 4.50-4.61 (m, 1H), 4.63-4.73 (m, 1H), 6.59 (s, 1H), 6.68-6.75 (m, 2H), 6.77-6.82 (m, 1H), 7.45-7.52 (m, 3H), 7.58-7.64 (m, 1H); ESIMS found for C₂₂H₂₁ClN₂O₃S: m/z 429.3 (M+1).

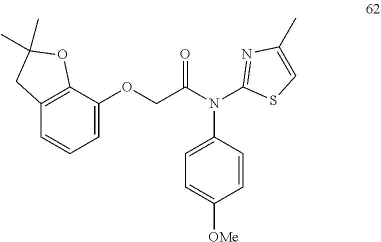

2-((2,2-Dimethyl-2,3-dihydrobenzofuran-7-yl)oxy)-N-(4-methoxyphenyl)-N-(4-methylthiazol-2-yl)acetamide (62) was prepared in accordance with the procedures described in Schemes 12 and 9. ¹H NMR (400 MHz, CHLOROFORM-d) δ ppm 1.46 (s, 6H), 2.25 (s, 3H), 3.00 (s, 2H), 3.89 (s, 3H), 4.66 (s, 2H), 6.59 (s, 1H), 6.70-6.74 (m, 3H), 7.05 (d, J=8 Hz, 2H), 7.29 (d, J=8 Hz, 2H); ESIMS found for C₂₃H₂₄N₂O₄S: m/z 425.1 (M+1).

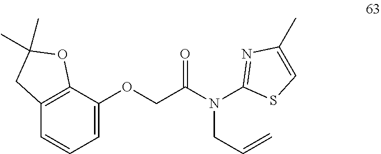

N-Allyl-2-((2,2-dimethyl-2,3-dihydrobenzofuran-7-yl)oxy)-N-(4-methylthiazol-2-yl)acetamide (63) was prepared in accordance with the procedures described in Schemes 12 and 9. ¹H NMR (400 MHz, CHLOROFORM-d) δ ppm 1.48 (s, 6H), 2.36 (d, J=0.73 Hz, 3H), 3.01 (s, 2H), 4.90 (br s, 2H), 5.05 (s, 2H), 5.21 (dd, J=10.27, 0.86 Hz, 1H), 5.25 (br d, J=2.32 Hz, 1H), 5.94-6.08 (m, 1H), 6.58 (br s, 1H), 6.70-6.76 (m, 1H), 6.76-6.80 (m, 1H), 6.80-6.86 (m, 1H); ESIMS found for C₁₉H₂₂N₂O₃S: m/z 359.3 (M+1).

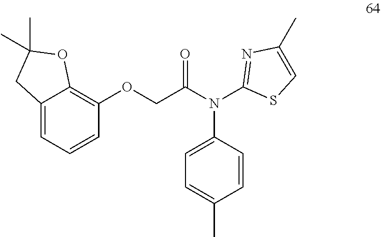

2-((2,2-Dimethyl-2,3-dihydrobenzofuran-7-yl)oxy)-N-(4-methylthiazol-2-yl)-N-(p-tolyl)acetamide (64) was prepared in accordance with the procedures described in Schemes 12 and 9. ¹H NMR (400 MHz, CHLOROFORM-d) δ ppm 1.43 (s, 6H), 2.22 (s, 3H), 2.43 (s, 3H), 2.96 (s, 2H), 4.62 (s, 2H), 6.56 (s, 1H), 6.65-6.73 (m, 2H), 6.74-6.79 (m, 1H), 7.19-7.27 (m, 2H), 7.32 (br d, J=7.95 Hz, 2H); ESIMS found for C₂₃H₂₄N₂O₃S: m/z 409.4 (M+1).

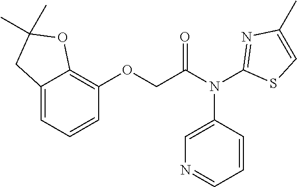

2-((2,2-Dimethyl-2,3-dihydrobenzofuran-7-yl)oxy)-N-(4-methylthiazol-2-yl)-N-(pyridin-3-yl)acetamide (65) was prepared in accordance with the procedures described in Schemes 12 and 9. ¹H NMR (400 MHz, CHLOROFORM-d) δ 8.72 (broad d, J=8 Hz, 1H), 8.62 (d, J=4 Hz, 1H), 7.70-7.70 (m, 1H), 7.48 (dd, J=8, 4 Hz, 1H), 6.80-6.74 (m, 1H), 6.73-6.67 (m, 2H), 6.60 (s, 1H), 4.66 (s, 2H), 2.96 (s, 2H), 2.20 (s, 3H), 1.40 (s, 6H); ESIMS found for C₂₁H₂₁N₃O₃S: m/z 396.2 (M+1).

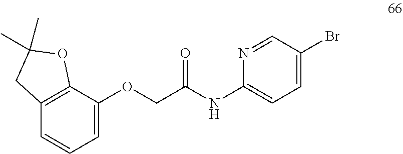 [SMILES: CC1(C)Cc2cccc(OCC(=O)Nc3ccc(Br)cn3)c2O1]

N-(5-Bromopyridin-2-yl)-2-((2,2-dimethyl-2,3-dihydrobenzofuran-7-yl)oxy) acetamide (66) was prepared in accordance with the procedure described in Scheme 9. 75% yield. ¹H NMR (400 MHz, CHLOROFORM-d) δ ppm 1.52 (s, 6H), 3.05 (s, 2H), 4.71 (s, 2H), 6.74-6.84 (m, 2H), 6.89 (dd, J=6.97, 1.34 Hz, 1H), 7.82 (dd, J=8.80, 2.45 Hz, 1H), 8.22 (d, J=8.93 Hz, 1H), 8.37 (d, J=2.45 Hz, 1H), 9.17 (br s, 1H); ESIMS found for C₁₇H₁₇BrN₂O₃: m/z 379.2 (Br⁸¹M+1).

The preparation of 2-((2,2-dimethyl-2,3-dihydrobenzofuran-7-yl)oxy)-N-(5-vinylpyridin-2-yl)acetamide (67) is depicted below in Scheme 14.

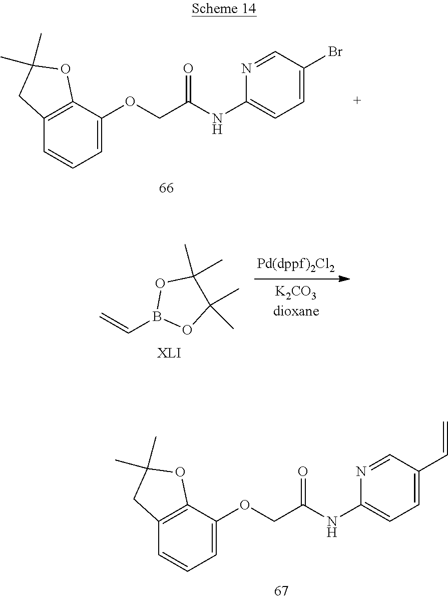 [SMILES: CC1(C)Cc2cccc(OCC(=O)Nc3ccc(Br)cn3)c2O1 ; C=CB1OC(C)(C)C(C)(C)O1 ; C=Cc1ccc(NC(=O)COc2cccc3c2OC(C)(C)C3)nc1]

N-(5-Bromopyridin-2-yl)-2-((2,2-dimethyl-2,3-dihydrobenzofuran-7-yl)oxy) acetamide (66) 200 mg, 0.530 mmol), 4,4,5,5-tetramethyl-2-vinyl-1,3,2-dioxaborolane (XLI) (122 mg, 0.795 mmol) and K₂CO₃ (220 mg, 1.59 mmol) in dioxane (5 mL) and water (1 mL) was added to a 15 mL sealed tube. The tube was evacuated under reduced pressure and backfilled with N₂. Pd(dppf)₂Cl₂.DCM (38.8 mg, 0.0530 mmol) was added and the tube kept under N₂. The reaction mixture was sealed with a Teflon screw cap and the mixture was heated at 100° C. for 18 h. The mixture was filtered through a pad of Celite®, and the filter cake was washed with EtOAc (50 mL) and concentrated under reduced pressure. Flash chromatography (EtOAc:hexanes, gradient elution from 0% EtOAc to 100% EtOAc) afforded 84 mg (49%) of 2-((2,2-dimethyl-2,3-dihydrobenzofuran-7-yl)oxy)-N-(5-vinylpyridin-2-yl)acetamide (67). ¹H NMR (400 MHz, CHLOROFORM-d) δ ppm 1.52 (s, 6H), 3.05 (s, 2H), 4.72 (s, 2H), 5.32 (d, J=11.25 Hz, 1H), 5.76 (d, J=17.61 Hz, 1H), 6.68 (dd, J=17.67, 11.07 Hz, 1H), 6.75-6.80 (m, 1H), 6.80-6.84 (m, 1H), 6.88 (dd, J=7.03, 1.28 Hz, 1H), 7.80 (dd, J=8.68, 2.32 Hz, 1H), 8.25 (d, J=8.68 Hz, 1H), 8.31 (d, J=2.20 Hz, 1H), 9.13 (br s, 1H); ESIMS found for C₁₉H₂₀N₂O₃: m/z 325.4 (M+1).

The synthesis of 2-((2,2-dimethyl-2,3-dihydrobenzofuran-7-yl)oxy)-N-(5-ethylpyridin-2-yl)acetamide (68) is depicted below in Scheme 15.

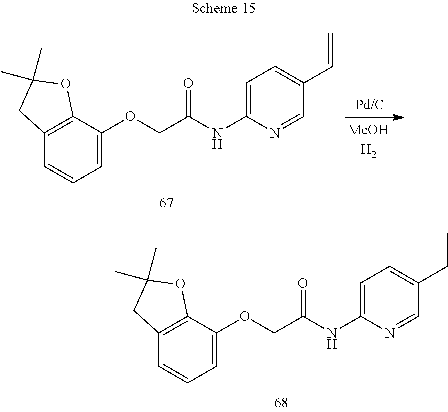

5% Palladium on carbon (20 mg) was added to a solution of 2-((2,2-dimethyl-2,3-dihydrobenzofuran-7-yl)oxy)-N-(5-vinylpyridin-2-yl)acetamide (67) (70.0 mg, 0.216 mmol) in MeOH (5 mL) and flushed with H₂ (3×). The mixture was placed under a balloon of hydrogen and stirred overnight (>16 h). The mixture was filtered through a pad of Celite®, rinsed with EtOAc (50 mL), and the filtrate was concentrated under reduced pressure. Flash chromatography (EtOAc:hexanes, gradient elution from 0% EtOAc to 100% EtOAc) afforded 30 mg (43%) of 2-((2,2-dimethyl-2,3-dihydrobenzofuran-7-yl)oxy)-N-(5-ethylpyridin-2-yl) acetamide (68). ¹H NMR (400 MHz, CHLOROFORM-d) δ ppm 1.22-1.27 (m, 3H), 1.52 (s, 6H), 2.63 (q, J=7.58 Hz, 2H), 3.04 (s, 2H), 4.71 (s, 2H), 6.72-6.84 (m, 2H), 6.87 (dd, J=6.97, 1.22 Hz, 1H), 7.56 (dd, J=8.44, 2.32 Hz, 1H), 8.15 (d, J=1.83 Hz, 1H), 8.18 (d, J=8.44 Hz, 1H), 9.04 (br s, 1H); ESIMS found for C₁₉H₂₂N₂O₃: m/z 327.1 (M+1).

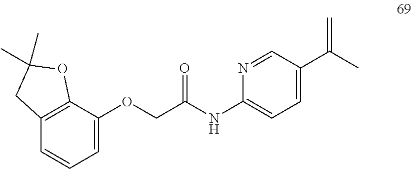 [SMILES: C=C(C)c1ccc(NC(=O)COc2cccc3c2OC(C)(C)C3)nc1]

2-((2,2-Dimethyl-2,3-dihydrobenzofuran-7-yl)oxy)-N-(5-(prop-1-en-2-yl) pyridin-2-yl)acetamide (69) was prepared in accordance with the procedures described in Scheme 14 from 4,4,5,5-tetramethyl-2-(prop-1-en-2-yl)-1,3,2-dioxaborolane. 55% yield. ¹H NMR (400 MHz, CHLOROFORM-d) δ ppm 1.53 (s, 6H), 2.16 (s, 3H), 3.05 (s, 2H), 4.72 (s, 2H), 5.13 (s, 1H), 5.39 (s, 1H), 6.73-6.85 (m, 2H), 6.88 (dd, J=7.09, 0.73 Hz, 1H), 7.80 (dd, J=8.68, 2.45 Hz, 1H), 8.23 (d, J=8.68 Hz, 1H), 8.42 (d, J=2.32 Hz, 1H), 9.12 (br s, 1H); ESIMS found for C₂₀H₂₂N₂O₃: m/z 339.1 (M+1).

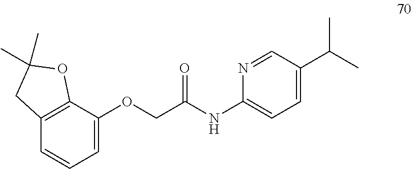 [SMILES: CC(C)c1ccc(NC(=O)COc2cccc3c2OC(C)(C)C3)nc1]

2-((2,2-Dimethyl-2,3-dihydrobenzofuran-7-yl)oxy)-N-(5-isopropylpyridin-2-yl)acetamide (70) was prepared in accordance with the procedures described in Scheme 15. ¹H NMR (400 MHz, CHLOROFORM-d) δ ppm 1.27 (d, J=6.85 Hz, 6H), 1.52 (s, 6H), 2.85-2.98 (m, 1H), 3.04 (s, 2H), 4.71 (s, 2H), 6.71-6.83 (m, 2H), 6.86 (dd, J=7.03, 1.16 Hz, 1H), 7.53-7.62 (m, 1H), 8.17 (s, 1H), 8.18 (d, J=5.99 Hz, 1H), 9.05 (br s, 1H); ESIMS found for C₂₀H₂₄N₂O₃: m/z 341.3 (M+1).

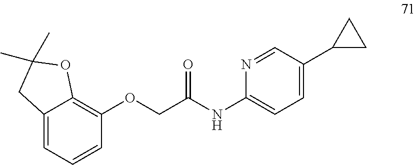 [SMILES: CC1(C)Cc2cccc(OCC(=O)Nc3ccc(C4CC4)cn3)c2O1]

N-(5-Cyclopropylpyridin-2-yl)-2-((2,2-dimethyl-2,3-dihydrobenzofuran-7-yl)oxy)acetamide (71) was prepared in accordance with the procedures described in Scheme 14 from compound 66 and cyclopropylboronic acid. 25% yield. ¹H NMR (400 MHz, CHLOROFORM-d) δ ppm 0.64-0.73 (m, 2H), 0.94-1.03 (m, 2H), 1.51 (s, 6H), 1.87 (tt, J=8.48, 5.03 Hz, 1H), 3.03 (s, 2H), 4.69 (s, 2H), 6.72-6.78 (m, 1H), 6.78-6.83 (m, 1H), 6.86 (dd, J=7.03, 1.28 Hz, 1H), 7.35 (dd, J=8.56, 2.45 Hz, 1H), 8.13 (br d, J=8.93 Hz, 2H), 9.03 (br s, 1H); ESIMS found for C₂₀H₂₂N₂O₃: m/z 339.2. (M+1).

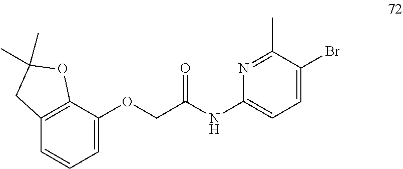 [SMILES: Cc1nc(NC(=O)COc2cccc3c2OC(C)(C)C3)ccc1Br]

N-(5-Bromo-6-methylpyridin-2-yl)-2-((2,2-dimethyl-2,3-dihydrobenzofuran-7-yl)oxy)acetamide (72) was prepared in accordance with the procedures described in Scheme 9. 31% yield. ¹H NMR (400 MHz, CHLOROFORM-d) δ ppm 1.53 (s, 6H), 2.57 (s, 3H), 3.06 (s, 2H), 4.70 (s, 2H), 6.72-6.81 (m, 1H), 6.81-6.86 (m, 1H), 6.89 (dd, J=7.15, 1.28 Hz, 1H), 7.79 (d, J=8.68 Hz, 1H), 8.00 (d, J=8.68 Hz, 1H), 9.12 (br s, 1H); ESIMS found for C₁₈H₁₉BrN₂O₃: m/z 392.9 (Br⁸¹M+1).

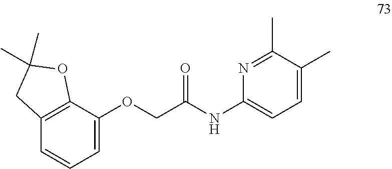

2-((2,2-Dimethyl-2,3-dihydrobenzofuran-7-yl)oxy)-N-(5,6-dimethylpyridin-2-yl)acetamide (73) was prepared in accordance with the procedures described in Scheme 14 from compound 72 and trimethylboroxine. 36% yield. ¹H NMR (400 MHz, CHLOROFORM-d) δ ppm 1.53 (s, 6H), 2.25 (s, 3H), 2.41 (s, 3H), 3.05 (s, 2H), 4.70 (s, 2H), 6.71-6.80 (m, 1H), 6.80-6.85 (m, 1H), 6.87 (dd, J=7.15, 1.04 Hz, 1H), 7.43 (d, J=8.31 Hz, 1H), 7.98 (d, J=8.31 Hz, 1H), 9.00 (br s, 1H); ESIMS found for C₁₉H₂₂N₂O₃: m/z 327.1 (M+1).

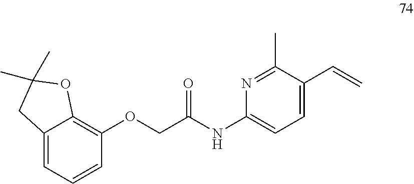 [SMILES: C=Cc1ccc(NC(=O)COc2cccc3c2OC(C)(C)C3)nc1C]

2-((2,2-Dimethyl-2,3-dihydrobenzofuran-7-yl)oxy)-N-(6-methyl-5-vinylpyridin-2-yl)acetamide (74) was prepared in accordance with the procedures described in Scheme 14 from compound 72. 64% yield. ¹H NMR (400 MHz, CHLOROFORM-d) δ ppm 1.52 (s, 6H), 2.49 (s, 3H), 3.04 (s, 2H), 4.70 (s, 2H), 5.33 (dd, J=11.00, 0.86 Hz, 1H), 5.62 (dd, J=17.36, 0.98 Hz, 1H), 6.73-6.83 (m, 2H), 6.83-6.91 (m, 2H), 7.78 (d, J=8.56 Hz, 1H), 8.07 (d, J=8.56 Hz, 1H), 9.08 (br s, 1H); ESIMS found for C₂₀H₂₂N₂O₃: m/z 339.4 (M+1).

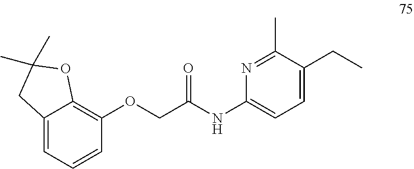 [SMILES: CCc1ccc(NC(=O)COc2cccc3c2OC(C)(C)C3)nc1C]

2-((2,2-Dimethyl-2,3-dihydrobenzofuran-7-yl)oxy)-N-(5-ethyl-6-methylpyridin-2-yl)acetamide (75) was prepared in accordance with the procedures described in Scheme 15 from compound 74. 69% yield. ¹H NMR (400 MHz, CHLOROFORM-d) δ ppm 1.20 (t, J=7.52 Hz, 3H), 1.52 (s, 6H), 2.44 (s, 3H), 2.60 (q, J=7.54 Hz, 2H), 3.04 (s, 2H), 4.69 (s, 2H), 6.73-6.79 (m, 1H), 6.79-6.84 (m, 1H), 6.86 (dd, J=7.15, 1.16 Hz, 1H), 7.46 (d, J=8.31 Hz, 1H), 8.00 (d, J=8.31 Hz, 1H), 8.99 (br s, 1H); ESIMS found for C₂₀H₂₄N₂O₃: m/z 341.3 (M+1).

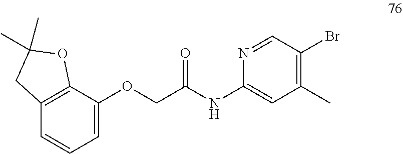

N-(5-Bromo-4-methylpyridin-2-yl)-2-((2,2-dimethyl-2,3-dihydrobenzofuran-7-yl)oxy)acetamide (76) was prepared in accordance with the procedures described in Scheme 8. 34% yield. ¹H NMR (400 MHz, CHLOROFORM-d) δ ppm 1.52 (s, 6H), 2.44 (s, 3H), 3.05 (s, 2H), 4.71 (s, 2H), 6.79 (quin, J=7.12 Hz, 2H), 6.88 (dd, J=6.97, 1.22 Hz, 1H), 8.22 (s, 1H), 8.33 (s, 1H), 9.09 (br s, 1H); ESIMS found for C₁₈H₁₉BrN₂O₃: m/z 391.3 (M+1).

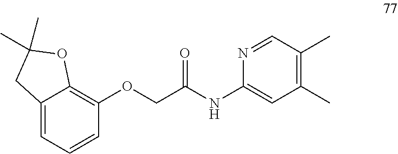

2-((2,2-Dimethyl-2,3-dihydrobenzofuran-7-yl)oxy)-N-(4,5-dimethylpyridin-2-yl)acetamide (77) was prepared in accordance with the procedures described in Scheme 14 from compound 76 and trimethylboroxine. ESIMS found for C₁₉H₂₂N₂O₃: m/z 327.5 (M+1).

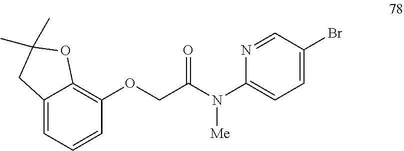 [SMILES: CN(C(=O)COc1cccc2c1OC(C)(C)C2)c1ccc(Br)cn1]

N-(5-Bromopyridin-2-yl)-2-((2,2-dimethyl-2,3-dihydrobenzofuran-7-yl) oxy)-N-methylacetamide (78) was prepared in 52% yield from 5-bromo-N-methylpyridin-2-amine in accordance with the procedures described in Scheme 9. ESIMS found for C₁₈H₁₉⁷⁹BrN₂O₃: m/z 391.3 (M+1).

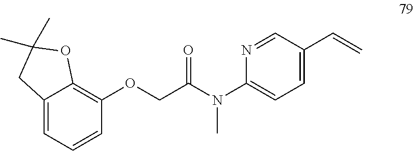 [SMILES: C=Cc1ccc(N(C)C(=O)COc2cccc3c2OC(C)(C)C3)nc1]

2-((2,2-Dimethyl-2,3-dihydrobenzofuran-7-yl)oxy)-N-methyl-N-(5-vinylpyridin-2-yl)acetamide (79) was prepared from compound 78 in accordance with the procedures described in Scheme 14 in 89% yield. ESIMS found for C₂₀H₂₂N₂O₃: m/z 339.1 (M+1).

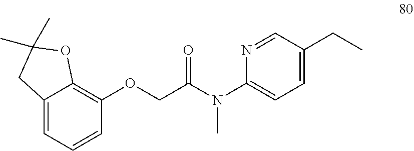 [SMILES: CCc1ccc(N(C)C(=O)COc2cccc3c2OC(C)(C)C3)nc1]

2-((2,2-Dimethyl-2,3-dihydrobenzofuran-7-yl)oxy)-N-(5-ethylpyridin-2-yl)-N-methylacetamide (80) was prepared in accordance with the procedures described in Scheme 15 from compound 79. 64% yield. ¹H NMR (400 MHz, CHLOROFORM-d) δ ppm 1.26 (t, J=7.64 Hz, 3H), 1.44 (s, 6H), 2.66 (q, J=7.58 Hz, 2H), 2.97 (s, 2H), 3.38 (s, 3H), 4.83 (s, 2H), 6.64-6.72 (m, 2H), 6.72-6.78 (m, 1H), 7.18 (br d, J=5.38 Hz, 1H), 7.57 (dd, J=8.19, 2.45 Hz, 1H), 8.27 (d, J=2.20 Hz, 1H); ESIMS found for C₂₀H₂₄N₂O₃: m/z 341.1 (M+1).

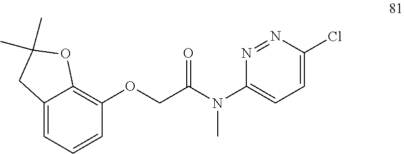 [SMILES: CN(C(=O)COc1cccc2c1OC(C)(C)C2)c1ccc(Cl)nn1]

N-(6-Chloropyridazin-3-yl)-2-((2,2-dimethyl-2,3-dihydrobenzofuran-7-yl) oxy)-N-methylacetamide (81) was prepared in accordance with the procedures described in Scheme 9 in 15% yield. ESIMS found for C₁₇H₁₈ClN₃O₃: m/z 347.9 (M+1).

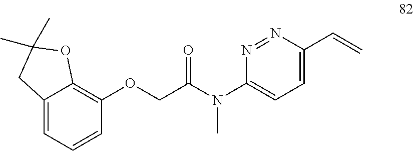 [SMILES: C=Cc1ccc(N(C)C(=O)COc2cccc3c2OC(C)(C)C3)nn1]

2-((2,2-Dimethyl-2,3-dihydrobenzofuran-7-yl)oxy)-N-methyl-N-(6-vinylpyridazin-3-yl)acetamide (82) was prepared in accordance with the procedures described in Scheme 14 in 83% yield. ESIMS found for C₁₉H₂₁N₃O₃: m/z 340.2 (M+1).

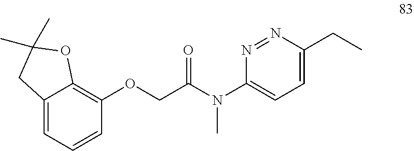 [SMILES: CCc1ccc(N(C)C(=O)COc2cccc3c2OC(C)(C)C3)nn1]

2-((2,2-Dimethyl-2,3-dihydrobenzofuran-7-yl)oxy)-N-(6-ethylpyridazin-3-yl)-N-methylacetamide (83) was prepared in accordance with the procedures described in Scheme 15. 55% yield. ¹H NMR (400 MHz, CHLOROFORM-d) δ ppm 1.37 (t, J=7.58 Hz, 3H), 1.45 (s, 6H), 2.95-3.04 (m, 4H), 3.54 (s, 3H), 4.97 (s, 2H), 6.68-6.74 (m, 2H), 6.74-6.80 (m, 1H), 7.34 (d, J=9.05 Hz, 1H), 7.67 (br s, 1H); ESIMS found for C₁₉H₂₃N₃O₃: m/z 342.4 (M+1).

The synthesis of intermediate 6-vinylpyridazin-3-amine (XLIII) is depicted below in Scheme 16.

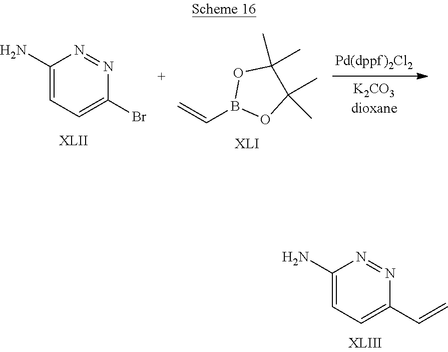

A mixture of 6-bromopyridazin-3-amine (XLII) (500 mg, 2.87 mmol), 4,4,5,5-tetramethyl-2-vinyl-1,3,2-dioxaborolane (XLI) (664 mg, 4.31 mmol) K₂CO₃ (1.19 g, 8.62 mmol), dioxane (5 mL) and water (1 mL) in a 15 mL sealed tube was evacuated under reduced pressure and filled with N2. Pd(dppf)₂Cl₂.DCM (210 mg, 0.287 mmol) was added, and the vessel flushed with N2. The reaction mixture was sealed with a Teflon screw cap and the mixture was heated at 80° C. for 18 h then filtered through a pad of Celite®. The filter cake was washed with EtOAc (50 mL), and concentrated under reduced pressure. Flash chromatography (EtOAc:hexanes, gradient elution) afforded 125 mg (36%) of 6-vinylpyridazin-3-amine (XLIII). ESIMS found for C₆H₇N₃: m/z 122.2 (M+1).

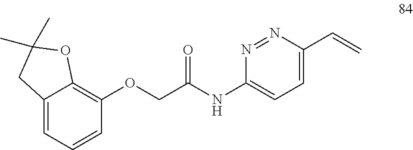 [SMILES: C=Cc1ccc(NC(=O)COc2cccc3c2OC(C)(C)C3)nn1]

2-((2,2-Dimethyl-2,3-dihydrobenzofuran-7-yl)oxy)-N-(6-vinylpyridazin-3-yl)acetamide (84) was prepared in accordance with the procedures described in Scheme 9 and 16 in 48% yield. ¹H NMR (400 MHz, CHLOROFORM-d) δ ppm 1.53 (s, 6H), 3.06 (s, 2H), 4.76 (s, 2H), 5.64 (d, J=11.13 Hz, 1H), 6.16 (d, J=17.85 Hz, 1H), 6.73-6.87 (m, 2H), 6.89 (dd, J=7.09, 1.10 Hz, 1H), 7.03 (dd, J=17.79, 11.07 Hz, 1H), 7.64 (d, J=9.29 Hz, 1H), 8.50 (d, J=9.17 Hz, 1H), 9.64 (br s, 1H); ESIMS found for C₁₈H₁₉N₃O₃: m/z 326.5 (M+1).

The synthesis of intermediate 6-ethylpyridazin-3-amine (XLIV) is depicted below in Scheme 17.

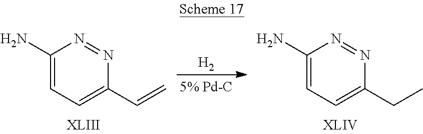

To a solution of 6-vinylpyridazin-3-amine (XLIII) (100 mg, 0.825 mmol) in MeOH (5 mL) was added 5% Pd—C (10 mg). The mixture was purged with N2 (3×), then purged with H2, and stirred under a balloon of H2 overnight. The mixture was filtered through a pad of Celite®, rinsed with EtOAc (50 mL), and concentrated under reduced pressure. Flash chromatography (EtOAc:hexanes, gradient elution) afforded 75 mg (74%) of 6-ethylpyridazin-3-amine (XLIV). ESIMS found for C₆H₉N₃: m/z 124.1 (M+1)

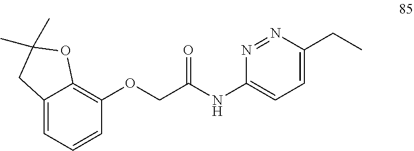 [SMILES: CCc1ccc(NC(=O)COc2cccc3c2OC(C)(C)C3)nn1]

2-((2,2-Dimethyl-2,3-dihydrobenzofuran-7-yl)oxy)-N-(6-ethylpyridazin-3-yl)acetamide (85) was prepared in accordance with the procedures described in Scheme 9 and 17 in 41% yield. ¹H NMR (400 MHz, CHLOROFORM-d) δ ppm 1.36 (t, J=7.64 Hz, 3H), 1.53 (s, 6H), 2.99 (q, J=7.66 Hz, 2H), 3.05 (s, 2H), 4.75 (s, 2H), 6.74-6.85 (m, 2H), 6.89 (dd, J=7.15, 1.04 Hz, 1H), 7.37 (d, J=9.17 Hz, 1H), 8.44 (d, J=9.17 Hz, 1H), 9.55 (br s, 1H); ESIMS found for C₁₈H₂₁N₃O₃: m/z 328.4 (M+1).

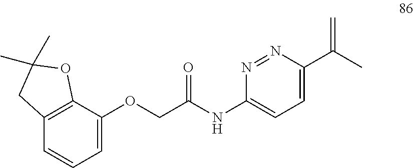 [SMILES: C=C(C)c1ccc(NC(=O)COc2cccc3c2OC(C)(C)C3)nn1]

2-((2,2-Dimethyl-2,3-dihydrobenzofuran-7-yl)oxy)-N-(6-(prop-1-en-2-yl) pyridazin-3-yl)acetamide (86) was prepared in accordance with the procedures described in Schemes 16 and 9. ¹H NMR (400 MHz, CHLOROFORM-d) δ ppm 1.52 (s, 6H), 2.32 (s, 3H), 3.05 (s, 2H), 4.76 (s, 2H), 5.39-5.47 (m, 1H), 5.81 (s, 1H), 6.74-6.81 (m, 1H), 6.81-6.85 (m, 1H), 6.89 (dd, J=7.03, 1.28 Hz, 1H), 7.73 (d, J=9.41 Hz, 1H), 8.48 (d, J=9.29 Hz, 1H), 9.59 (br s, 1H); ESIMS found for C₁₉H₂₁N₃O₃: m/z 340.3 (M+1).

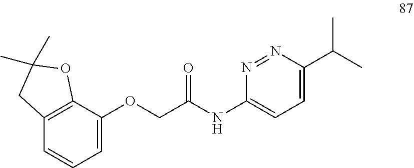 [SMILES: CC(C)c1ccc(NC(=O)COc2cccc3c2OC(C)(C)C3)nn1]

2-((2,2-Dimethyl-2,3-dihydrobenzofuran-7-yl)oxy)-N-(6-isopropylpyridazin-3-yl)acetamide (87) was prepared in accordance with the procedures described in Schemes 16, 17 and 9. ¹H NMR (400 MHz, CHLOROFORM-d) δ ppm 1.37 (d, J=6.97 Hz, 6H), 1.53 (s, 6H), 3.05 (s, 2H), 3.30 (spt, J=6.91 Hz, 1H), 4.75 (s, 2H), 6.75-6.80 (m, 1H), 6.80-6.84 (m, 1H), 6.88 (dd, J=6.97, 1.22 Hz, 1H), 7.39 (d, J=9.17 Hz, 1H), 8.44 (d, J=9.29 Hz, 1H), 9.53 (br s, 1H); ESIMS found for C₁₉H₂₃N₃O₃: m/z 342.3 (M+1).

The synthesis of intermediate 6-methyl-N-(p-tolyl)pyridazin-3-amine (XLVII), which describes the nucleophilic substitution of a heteroaryl halide with an amine, is depicted below in Scheme 18.

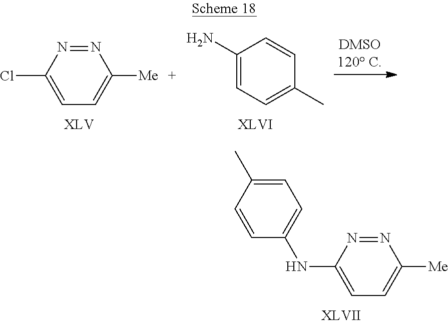

A solution of 3-chloro-6-methylpyridazine (XLV) (100 mg, 0.778 mmol) and p-toluidine (XLVI) (72.4 mg, 0.778 mmol) in DMSO (5 mL) was heated at 120° C. for 24 h. The bulk of the DMSO was removed under reduced pressure. The residue was dissolved in EtOAc, and the organic layer was washed with satd. aqueous NaHCO₃, dried over MgSO4, filtered and concentrated under reduced pressure. Flash chromatography (EtOAc:hexanes, gradient elution) afforded 72 mg (16%) of 6-methyl-N-(p-tolyl)pyridazin-3-amine (XLVII). ESIMS found for C₁₂H₁₃N₃: m/z 200 (M+H). This reaction can also be conducted in isopropanol in a sealed tube.

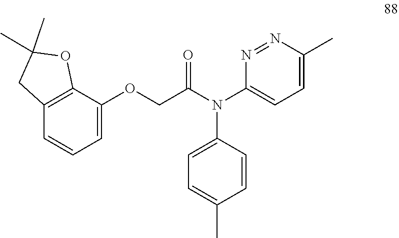

2-((2,2-Dimethyl-2,3-dihydrobenzofuran-7-yl)oxy)-N-(6-methylpyridazin-3-yl)-N-(p-tolyl)acetamide (88) was prepared in accordance with the amide coupling procedure described in in Scheme 9 and 18. 9.5% yield. ¹H NMR (400 MHz, CHLOROFORM-d) δ ppm 1.44 (s, 6H), 2.37 (s, 3H), 2.65 (s, 3H), 2.97 (s, 2H), 4.82 (s, 2H), 6.67-6.75 (m, 2H), 6.74-6.79 (m, 1H), 7.18-7.21 (m, 2H), 7.21-7.25 (m, 2H), 7.28 (d, J=9.05 Hz, 1H), 7.72 (br d, J=7.95 Hz, 1H); ESIMS found for C₂₄H₂₅N₃O₃: m/z 404.4 (M+1).

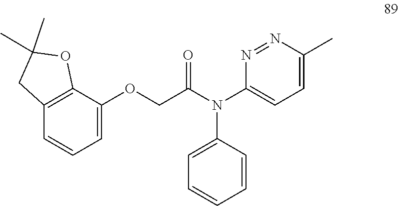

2-((2,2-Dimethyl-2,3-dihydrobenzofuran-7-yl)oxy)-N-(6-methylpyridazin-3-yl)-N-phenylacetamide (89) was prepared in accordance with the procedures described in Schemes 18 and 9. ¹H NMR (400 MHz, CHLOROFORM-d) δ ppm 1.44 (s, 6H), 2.66 (s, 3H), 2.97 (s, 2H), 4.82 (s, 2H), 6.67-6.75 (m, 2H), 6.74-6.82 (m, 1H), 7.27 (d, J=9.17 Hz, 1H), 7.32 (d, J=6.60 Hz, 2H), 7.38 (d, J=7.21 Hz, 1H), 7.40-7.48 (m, 2H), 7.69 (br d, J=8.19 Hz, 1H); ESIMS found for C₂₃H₂₃N₃O₃: m/z 390.3 (M+1).

The synthesis of intermediate 4-methyl-N-(pyrazin-2-yl)thiazol-2-amine (L) is depicted in Scheme 19.

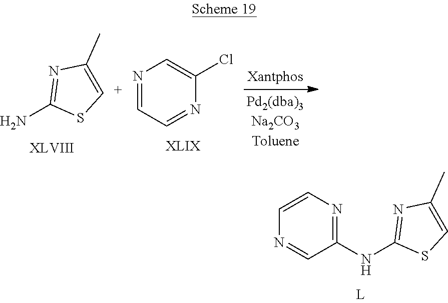

A mixture of 4-methylthiazol-2-amine (XLVIII) (159 mg, 1.39 mmol), 2-chloropyrazine (XLIX) (114 mg, 1.00 mmol), Na₂CO₃ (148 mg, 1.39 mmol) in toluene (5 mL) in a 15 mL sealed tube was evacuated under reduced pressure and backfilled with N2. To this mixture, Pd₂(dba)₃ (36.5 mg, 0.040 mmol) and xantphos (34.6 mg, 0.060 mmol) was added, and the system flushed with nitrogen. The reaction mixture was sealed with a Teflon screw cap and the mixture was heated at 100° C. for 24 h. The mixture was diluted with THE (10 mL), and was filtered through a pad of Celite®. The filter cake was washed with THE (50 mL) and concentrated under reduced pressure. Flash chromatography (EtOAc:hexanes, gradient elution) afforded 98 mg (43%) of 4-methyl-N-(pyrazin-2-yl)thiazol-2-amine (L). ESIMS found for C₈H₈N₄S: m/z 193 (M+1).

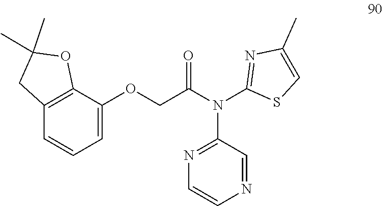 [SMILES: Cc1csc(N(C(=O)COc2cccc3c2OC(C)(C)C3)c2cnccn2)n1]

Compound 90 was prepared in accordance with the procedures described in Scheme 8 and 19. ¹H NMR (400 MHz, CHLOROFORM-d) δ ppm 1.44 (s, 6H), 2.25 (d, J=0.98 Hz, 3H), 2.97 (s, 2H), 4.82 (s, 2H), 6.63-6.74 (m, 3H), 6.76-6.82 (m, 1H), 8.54 (dd, J=2.51, 1.41 Hz, 1H), 8.62 (d, J=2.57 Hz, 1H), 8.81 (d, J=1.34 Hz, 1H); ESIMS found for C₂₀H₂₀N₄O₃S: m/z 397.3 (M+1).

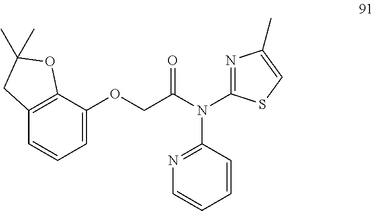 [SMILES: Cc1csc(N(C(=O)COc2cccc3c2OC(C)(C)C3)c2ccccn2)n1]

2-((2,2-Dimethyl-2,3-dihydrobenzofuran-7-yl)oxy)-N-(4-methylthiazol-2-yl)-N-(pyridin-2-yl)acetamide (91) was prepared from 2-chloropyridine in accordance with the procedures described in Schemes 19 and 9. ¹H NMR (400 MHz, CHLOROFORM-d) δ ppm 1.45 (s, 6H), 2.24 (d, J=0.86 Hz, 3H), 2.98 (s, 2H), 4.71 (s, 2H), 6.63 (d, J=0.86 Hz, 1H), 6.70-6.73 (m, 2H), 6.75-6.80 (m, 1H), 7.43 (ddd, J=7.46, 4.89, 0.86 Hz, 1H), 7.49 (d, J=7.95 Hz, 1H), 7.91 (td, J=7.70, 1.96 Hz, 1H), 8.63 (dt, J=4.89, 0.92 Hz, 1H); ESIMS found for C₂₁H₂₁N₃O₃S: m/z 396.2 (M+1).

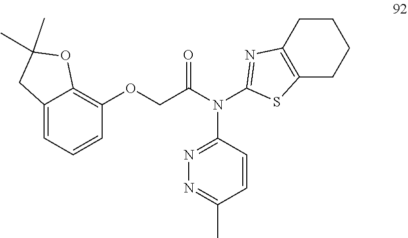 [SMILES: Cc1ccc(N(C(=O)COc2cccc3c2OC(C)(C)C3)c2nc3c(s2)CCCC3)nn1]

2-((2,2-Dimethyl-2,3-dihydrobenzofuran-7-yl)oxy)-N-(6-methylpyridazin-3-yl)-N-(4,5,6,7-tetrahydrobenzo[d]thiazol-2-yl)acetamide (92) was prepared from 3-chloro-6-methylpyridazine in accordance with the procedures described in Schemes 19 and 9. ¹H NMR (400 MHz, CHLOROFORM-d) δ ppm 1.45 (s, 6H), 1.79-1.86 (m, 4H), 2.53-2.59 (m, 2H), 2.72 (br s, 2H), 2.76 (s, 3H), 2.98 (s, 2H), 4.85 (s, 2H), 6.65-6.73 (m, 2H), 6.75-6.79 (m, 1H), 7.44 (d, J=8.80 Hz, 1H), 7.65 (d, J=8.80 Hz, 1H); ESIMS found for C₂₄H₂₆N₄O₃S: m/z 451.4 (M+1).

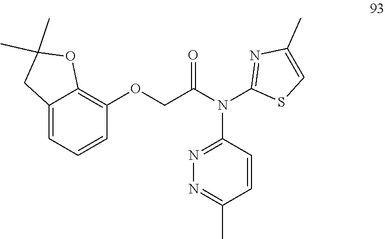 [SMILES: Cc1ccc(N(C(=O)COc2cccc3c2OC(C)(C)C3)c2nc(C)cs2)nn1]

2-((2,2-Dimethyl-2,3-dihydrobenzofuran-7-yl)oxy)-N-(6-methylpyridazin-3-yl)-N-(4-methylthiazol-2-yl)acetamide (93) was prepared from 3-chloro-6-methylpyridazine in accordance with the procedures described in Schemes 19 and 9. H NMR (400 MHz, CHLOROFORM-d) δ ppm 1.44 (s, 6H), 2.23 (s, 3H), 2.79 (s, 3H), 2.97 (s, 2H), 4.84 (s, 2H), 6.66 (br d, J=5.75 Hz, 2H), 6.68-6.73 (m, 1H), 6.75-6.80 (m, 1H), 7.48 (d, J=8.80 Hz, 1H), 7.63 (d, J=8.80 Hz, 1H); ESIMS found for C₂₁H₂₂N₄O₃S: m/z 411.4 (M+1).

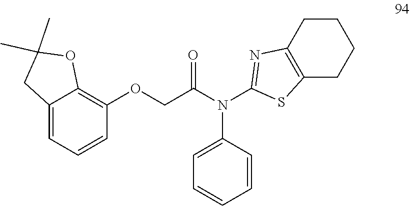 [SMILES: CC1(C)Cc2cccc(OCC(=O)N(c3ccccc3)c3nc4c(s3)CCCC4)c2O1]

2-((2,2-Dimethyl-2,3-dihydrobenzofuran-7-yl)oxy)-N-phenyl-N-(4,5,6,7-tetrahydrobenzo[d]thiazol-2-yl)acetamide (94) was prepared from iodobenzene in accordance with the procedures described in Schemes 19 and 9. ¹H NMR (400 MHz, CHLOROFORM-d) δ ppm 1.45 (s, 6H), 1.76-1.84 (m, 4H), 2.54 (br s, 2H), 2.70 (br s, 2H), 2.98 (s, 2H), 4.64 (br s, 2H), 6.71 (d, J=4.77 Hz, 2H), 6.75-6.80 (m, 1H), 7.33-7.38 (m, 2H), 7.46-7.57 (m, 3H); ESIMS found for C₂₅H₂₆N₂O₃S: m/z 435.4 (M+1).

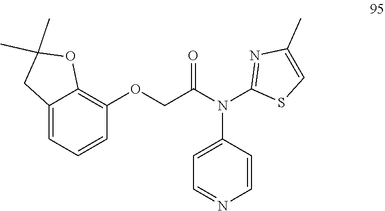 [SMILES: Cc1csc(N(C(=O)COc2cccc3c2OC(C)(C)C3)c2ccncc2)n1]

2-((2,2-Dimethyl-2,3-dihydrobenzofuran-7-yl)oxy)-N-(4-methylthiazol-2-yl)-N-(pyridin-4-yl)acetamide (100) was prepared in accordance with the procedures described in Schemes 19 and 9. ¹H NMR (400 MHz, CHLOROFORM-d) δ ppm 1.44 (s, 6H), 2.26 (s, 3H), 2.98 (s, 2H), 4.70 (s, 2H), 6.65-6.73 (m, 3H), 6.75-6.81 (m, 1H), 7.29-7.35 (m, 2H), 8.77 (d, J=5.75 Hz, 2H); ESIMS found for C₂₁H₂₁N₃O₃S: m/z 396.0 (M+1).

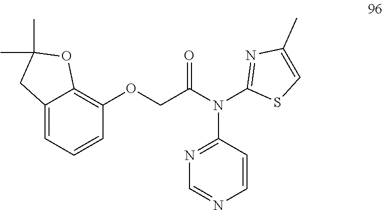 [SMILES: Cc1csc(N(C(=O)COc2cccc3c2OC(C)(C)C3)c2ccncn2)n1]

2-((2,2-Dimethyl-2,3-dihydrobenzofuran-7-yl)oxy)-N-(4-methylthiazol-2-yl)-N-(pyrimidin-4-yl)acetamide (96) was prepared in accordance with the procedures described in Schemes 19 and 9. ESIMS found for C₂₀H₂₀N₄O₃S: m/z 397.2 (M+1).

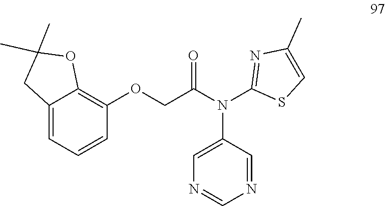 [SMILES: Cc1csc(N(C(=O)COc2cccc3c2OC(C)(C)C3)c2cncnc2)n1]

2-((2,2-Dimethyl-2,3-dihydrobenzofuran-7-yl)oxy)-N-(4-methylthiazol-2-yl)-N-(pyrimidin-5-yl)acetamide (97) was prepared in accordance with the procedures described in Schemes 19 and 9 from 5-bromopyrimidine except ^{t}BuBrettPhos (3.3 mol %), Pd₂dba₃ (0.75%), K₂CO₃ (1.4 eq) 3 Å sieves (200 mg/mmol) in 0.5 mL t-butanol was used in the initial Buchwald coupling reaction. ESIMS found for C₂₀H₂₀N₄O₃S: m/z 397.0 (M+1).

The synthesis of N-(Benzo[d]thiazol-2-yl)-2-((2,2-dimethyl-2,3-dihydrobenzofuran-7-yl)oxy)-N-phenylacetamide (98) is depicted in Scheme 20.

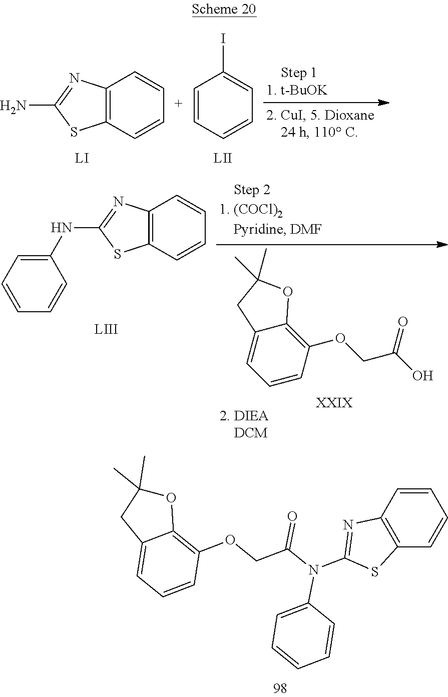

### Step 1

To a solution of benzo[d]thiazol-2-amine (LI) (1.00 g, 6.67 mmol) and anhydrous dioxane (8 mL) in a 50 mL glass sealed tube was added iodobenzene (LII) (1.50 g, 7.33 mmol), potassium tert-butoxide (1.50 g, 13.3 mmol), and copper iodide (0.13 g, 6.67 mmol). The reaction mixture was sealed with a teflon screw cap, and heated to 110° C. with stirring for 24 h. After cooling to rt, brine (5 mL) was added and mixture was diluted with EtOAc (20 mL). The organic layer was dried over Na₂SO₄, filtered, and concentrated under vacuum. Flash chromatography (EtOAc:hexanes, gradient elution from 0% EtOAc to 100% EtOAc) afforded 1.4 g (91%) of N-phenylbenzo[d]thiazol-2-amine (LIII). ESIMS found for C₁₃H₁₀N₂S: m/z 227.6 (M+1).

### Step 2

To a flame-dried round bottom flask was added 2-((2,2-dimethyl-2,3-dihydrobenzofuran-7-yl)oxy)acetic acid (XXIX) (100 mg, 0.45 mmol) and anhydrous DCM (5 mL), followed by anhydrous DMF (33 mg, 0.45 mmol) and anhydrous pyridine (36 mg, 0.45 mmol). The reaction mixture was cooled to 0° C., and oxalyl chloride (57 mg, 0.45 mmol) was added. After 10 min, a solution of N-phenylbenzo[d]thiazol-2-amine (56 mg, 0.45 mmol) and DIEA (58 mg, 0.45 mmol) in anhydrous DCM (1 mL) was added. After 1 h at rt, water (10 mL) was added and diluted with EtOAc (30 mL). The organic layer was washed consecutively with water (10 mL) and brine (10 mL). The organic layer was dried over Na₂SO₄, filtered, and concentrated under vacuum. Flash chromatography (EtOAc:hexanes, gradient elution from 0% EtOAc to 100% EtOAc) followed by prep-TLC (EtOAc:hexanes) afforded 24 mg (11%) of N-(benzo[d]thiazol-2-yl)-2-((2,2-dimethyl-2,3-dihydrobenzofuran-7-yl)oxy)-N-phenylacetamide (98). ¹H NMR (400 MHz, CHLOROFORM-d) δ ppm 1.50 (s, 6H), 3.00 (s, 2H), 5.04 (br s, 2H), 6.68-6.75 (m, 1H), 6.81 (dd, J=10.33, 7.89 Hz, 2H), 7.24 (br s, 1H), 7.31-7.39 (m, 6H), 7.39-7.45 (m, 2H); ESIMS found for C₂₅H₂₂N₂O₃S: m/z 431.1 (M+1).

The preparation of intermediate methyl 2-((4,5,6,7-tetrahydrobenzo[d] thiazol-2-yl)amino)acetate (LV) is depicted below in Scheme 21.

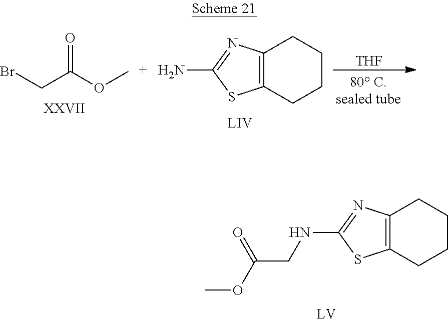

A solution of 4,5,6,7-tetrahydrobenzo[d]thiazol-2-amine (LIV) (0.302 g, 1.96 mmol) and methyl bromoacetate (XXVII) (0.300 g, 1.96 mmol) in 10 mL THE was heated at reflux until LCMS analysis showed the reaction to be complete. The mixture was treated with satd. NaHCO₃ (50 mL), and the mixture was extracted with EtOAc (50 mL). The organic layer was dried over MgSO4, filtered, and concentrated under reduced pressure. Flash chromatography (EtOAc:hexanes, gradient elution) afforded 0.175 g (39%) of methyl 2-((4,5,6,7-tetrahydrobenzo [d]thiazol-2-yl)amino)acetate (LV). ESIMS found for C₁₀H₁₄N₂O₂S: m/z 227.0 (M+H).

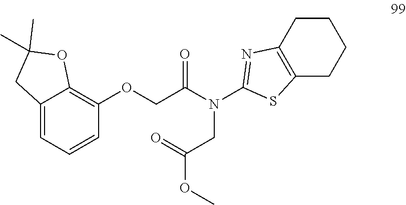 [SMILES: COC(=O)CN(C(=O)COc1cccc2c1OC(C)(C)C2)c1nc2c(s1)CCCC2]

Methyl 2-(2-((2,2-dimethyl-2,3-dihydrobenzofuran-7-yl)oxy)-N-(4,5,6,7-tetrahydrobenzo[d]thiazol-2-yl)acetamido)acetate (99) was prepared in accordance with the procedures described in Scheme 8 and 21 from methyl 2-((4,5,6,7-tetrahydrobenzo[d]thiazol-2-yl)amino)acetate (LV). ¹H NMR (400 MHz, CHLOROFORM-d) δ ppm 1.51 (s, 6H), 1.82-1.92 (m, 4H), 2.40 (br t, J=5.87 Hz, 2H), 2.51-2.57 (m, 2H), 3.01 (s, 2H), 3.71 (s, 3H), 4.80 (s, 2H), 4.82 (s, 2H), 6.65-6.68 (m, 2H), 6.70-6.74 (m, 1H); ESIMS found for C₂₂H₂₆N₂O₅S: m/z 431.4 (M+1).

The preparation of N-(4-cyanobenzo[d]thiazol-2-yl)-2-((2,2-dimethyl-2,3-dihydrobenzofuran-7-yl)oxy)acetamide (100) is depicted below in Scheme 22.

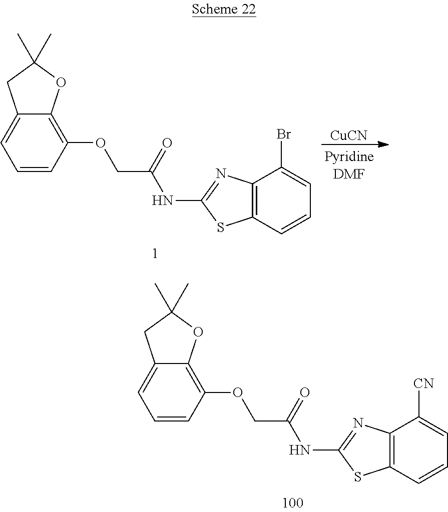

To a solution of N-(4-bromobenzo[d]thiazol-2-yl)-2-((2,2-dimethyl-2,3-dihydrobenzofuran-7-yl)oxy)acetamide (1) (50 mg, 0.12 mmol), pyridine (0.2 mL) and DMF (2 mL) was added CuCN (13.5 mg, 0.15 mmol) under N2. The reaction mixture was heated at reflux for 14 h, then the cooled reaction mixture was poured into a 20% aqueous solution of ethylene diamine. The mixture was extracted with ether, and the ether layer was washed with brine, dried over MgSO4, filtered, and concentrated under vacuum. The crude product was recrystallized from DCM/hexanes to afford 14 mg (32%) of N-(4-cyanobenzo[d]thiazol-2-yl)-2-((2,2-dimethyl-2,3-dihydrobenzofuran-7-yl)oxy)acetamide (100) as a white solid. ¹H NMR (400 MHz, CHLOROFORM-d) δ ppm 1.58 (s, 6H), 3.08 (s, 2H), 4.85 (s, 2H), 6.77-6.83 (m, 1H), 6.84-6.89 (m, 1H), 6.93 (br d, J=7.09 Hz, 1H), 7.38 (t, J=7.83 Hz, 1H), 7.77 (d, J=7.46 Hz, 1H), 8.04 (d, J=7.95 Hz, 1H), 10.76 (br s, 1H); ESIMS found for C₂₀H₁₇N₃O₃S: m/z 380.1 (M+1).

The synthesis of 2-((2,2-dimethyl-2,3-dihydrobenzofuran-7-yl)oxy)-N-(4-(pyridin-4-yl)benzo[d]thiazol-2-yl)acetamide (101) is depicted below in Scheme 23.

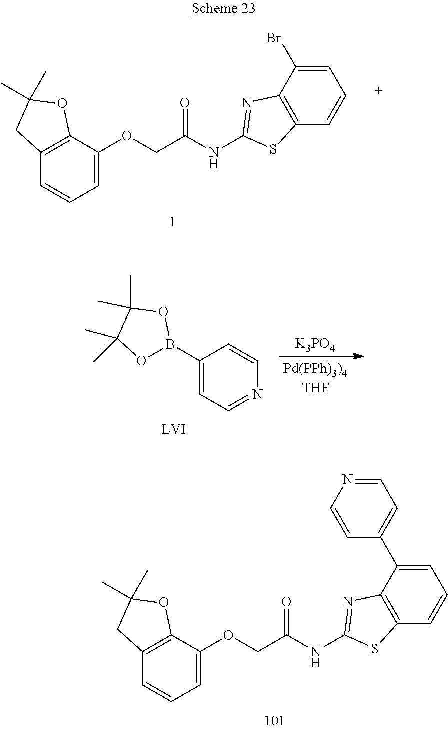

A mixture of N-(4-bromobenzo[d]thiazol-2-yl)-2-((2,2-dimethyl-2,3-dihydrobenzofuran-7-yl)oxy)acetamide (1) (50 mg, 0.12 mmol), 4-(4,4,5,5-tetramethyl-1,3,2-dioxaborolan-2-yl)pyridine (LVI) (30.8 mg, 0.15 mmol) K₃PO4 (85.7 mg, 0.404 mmol) in THE (5 mL) was purged with N2 for 2 min, then Pd(PPh₃)₄ (26.7 mg, 23 micromol)) was added. The reaction mixture was sealed with a Teflon screw cap and the mixture heated at 80° C. for 16 h. The mixture was diluted with EtOAc, washed sequentially with water and brine, dried over MgSO4, filtered and concentrated under reduced pressure. Flash chromatography (EtOAc:hexanes, gradient elution from 0% EtOAc to 100% EtOAc) afforded 4.2 mg (8.4%) of 2-((2,2-dimethyl-2,3-dihydrobenzofuran-7-yl)oxy)-N-(4-(pyridin-4-yl)benzo[d]thiazol-2-yl)acetamide (101). ESIMS found for C₂₄H₂₁N₃O₃S: m/z 432.6 (M+1).

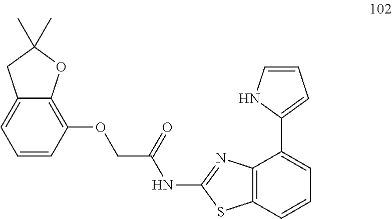 [SMILES: CC1(C)Cc2cccc(OCC(=O)Nc3nc4c(-c5ccc[nH]5)cccc4s3)c2O1]

N-(4-(1H-Pyrrol-2-yl)benzo[d]thiazol-2-yl)-2-((2,2-dimethyl-2,3-dihydrobenzofuran-7-yl)oxy)acetamide (102) was prepared in a manner similar to Scheme 23 except an additional step to hydrolyze an N-boc-group was added and is described below.

A mixture of N-(4-bromobenzo[d]thiazol-2-yl)-2-((2,2-dimethyl-2,3-dihydrobenzofuran-7-yl)oxy)acetamide (1) (60 mg, 0.14 mmol), (1-(tert-butoxycarbonyl)-1H-pyrrol-2-yl)boronic acid (38.1 mg, 0.18 mmol) K₃PO4 (103 mg, 0.486 mmol) in toluene (5 mL) was purged with N2 for 2 min, then Pd(PPh₃)₄ (32 mg, 28 micromol)) was added and the mixture heated at 110° C. for 6 h. The mixture was diluted with EtOAc, washed sequentially with water and brine, dried over MgSO4, filtered and concentrated under reduced pressure. Flash chromatography (EtOAc:hexanes, gradient elution) afforded 72 mg (55%) of tert-butyl 2-(2-(2-((2,2-dimethyl-2,3-dihydrobenzofuran-7-yl)oxy)acetamido)benzo[d]thiazol-4-yl)-1H-pyrrole-1-carboxylate, which was treated with 4N HCl in dioxane (0.17 mL, 0.68 mmoL) in DCM (1 mL). After stirring for 24 h, the mixture was diluted with DCM, washed sequentially with water and brine, dried over MgSO4, filtered, and concentrated under reduced pressure. Flash chromatography (EtOAc:hexanes, gradient elution from 0% EtOAc to 100% EtOAc) afforded 4.5 mg (13%) of N-(4-(1H-pyrrol-2-yl)benzo[d]thiazol-2-yl)-2-((2,2-dimethyl-2,3-dihydrobenzofuran-7-yl)oxy)acetamide (102). ESIMS found for C₂₃H₂₁N₃O₃S: m/z 420.0 (M+1).

The synthesis of intermediate 3-(2,2-dimethyl-2,3-dihydrobenzofuran-7-yl)propanoic acid (LXII) is depicted below in Scheme 24.

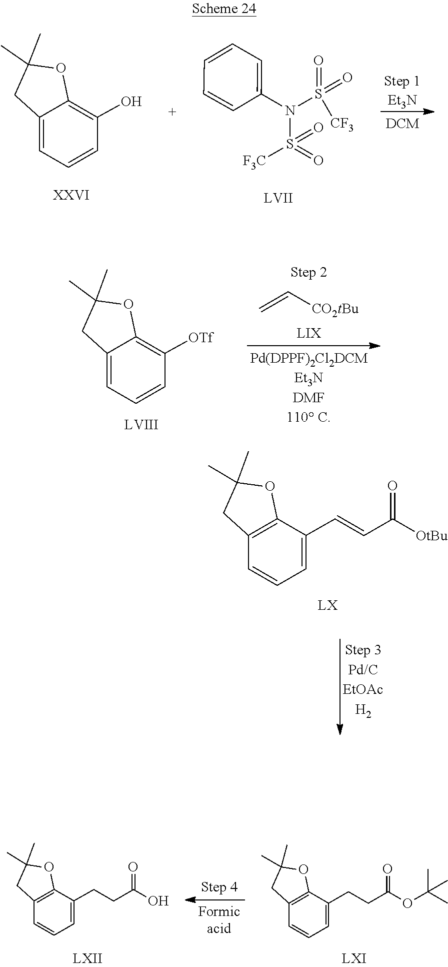

### Step 1

To a solution of 2,2-dimethyl-2,3-dihydrobenzofuran-7-ol (XXVI) (1.00 g, 6.09 mmol) and 1,1,1-trifluoro-N-phenyl-N-((trifluoromethyl)sulfonyl)methanesulfonamide (LVII) (2.39 g, 6.70 mmol) in 50 mL dry dichloromethane was added triethylamine (1.23 g, 12.2 mmol) at 0° C. and stirred overnight at rt. The reaction mixture was diluted with DCM (50 mL) and washed consecutively with satd. NaHCO₃ (40 mL), water (40 mL), and brine (40 mL), then dried (MgSO4), filtered and concentrated under vacuum. Flash chromatography (EtOAc:hexanes, gradient elution) afforded 1.7 g (94%) of 2,2-dimethyl-2,3-dihydrobenzofuran-7-yl trifluoromethanesulfonate (LVIII). ESIMS found for C₁₁H₁₁F₃O₄S: m/z 297.6.

### Step 2

To a solution of 2,2-dimethyl-2,3-dihydrobenzofuran-7-yl trifluoromethanesulfonate (LVIII) (1.70 g, 5.74 mmol) and tert-butyl acrylate (LIX) (7.35 g, 57.4 mmol) in DMF (17 mL) was added consecutively triethylamine (1.16 g, 11.5 mmol), and Pd(dppf)₂Cl₂.DCM (0.84 g, 1.1 mmol). The reaction mixture was equipped with a reflux condenser and was purged with N₂ then placed under a balloon of N₂ and heated to 110° C. for 18 h. The cooled reaction mixture was diluted with EtOAc (200 mL), and washed consecutively with satd. NaHCO₃ (100 mL), water (100 mL×2), brine (50 mL), dried (MgSO4), filtered and concentrated under vacuum. Flash chromatography (EtOAc:hexanes, gradient elution) afforded 1.1 g (70%) of (E)-tert-butyl 3-(2,2-dimethyl-2,3-dihydrobenzofuran-7-yl)acrylate (LX). ESIMS found for C₁₇H₂₂O₃: m/z 297.1 (M+Na).

### Step 3

To a solution of (E)-tert-butyl 3-(2,2-dimethyl-2,3-dihydrobenzofuran-7-yl)acrylate (LX) (1.10 g, 4.16 mmol) in EtOAc (10 mL) was add 5% Pd—C (0.20 g). The reaction flask was evacuated and refilled with N₂ (repeated 3×) followed by H2 (repeated 3×), then was stirred under a balloon of H₂ overnight. The reaction mixture was filtered through a pad of Celite®, and concentrated under vacuum to afford 1.06 g (92%) of tert-butyl 3-(2,2-dimethyl-2,3-dihydrobenzofuran-7-yl)propanoate (LXI). ESIMS found for C₁₇H₂₄O₃ m/z 299.4 (M+Na).

### Step 4

A solution of tert-butyl 3-(2,2-dimethyl-2,3-dihydrobenzofuran-7-yl)propanoate (LXI) (276 mg, 1.00 mmol) in formic acid (2.76 mL) was stirred at rt for 15 h. The reaction mixture was concentrated to a solid under vacuum, then purified by flash chromatography (EtOAc:hexanes, gradient elution) to afford 190 mg (86%) of 3-(2,2-dimethyl-2,3-dihydrobenzofuran-7-yl)propanoic acid (LXII). ESIMS found for C₁₃H₁₆O₃: m/z 221.3 (M+H).

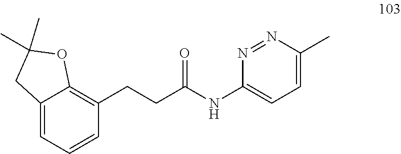

3-(2,2-Dimethyl-2,3-dihydrobenzofuran-7-yl)-N-(6-methylpyridazin-3-yl) propanamide (103) was prepared in accordance with the procedures described in Scheme 8 and 24 from 3-(2,2-dimethyl-2,3-dihydrobenzofuran-7-yl)propanoic acid and 6-methylpyridazin-3-amine. 18% yield. ¹H NMR (400 MHz, CHLOROFORM-d) 6 ppm 1.47 (s, 6H), 2.63 (s, 3H), 2.79-2.87 (m, 2H), 2.95-3.04 (m, 4H), 6.74 (t, J=7.46 Hz, 1H), 6.94-7.04 (m, 2H), 7.32 (d, J=9.17 Hz, 1H), 8.40 (d, J=9.17 Hz, 1H), 9.16 (br s, 1H); ESIMS found for C₁₈H₂₁N₃O₂: m/z 312.3 (M+1).

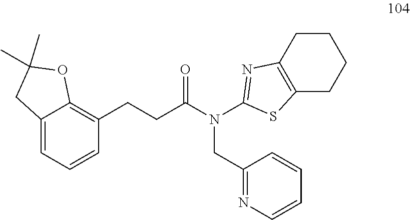 [SMILES: CC1(C)Cc2cccc(CCC(=O)N(Cc3ccccn3)c3nc4c(s3)CCCC4)c2O1]

3-(2,2-Dimethyl-2,3-dihydrobenzofuran-7-yl)-N-(pyridin-2-ylmethyl)-N-(4,5,6,7-tetrahydrobenzo[d]thiazol-2-yl)propanamide (104) was prepared in accordance with the procedures described in Schemes 24 and 7. 42% yield. ¹H NMR (400 MHz, CHLOROFORM-d) δ ppm 1.34 (s, 6H), 1.78-1.87 (m, 4H), 2.58 (br s, 2H), 2.68 (br s, 2H), 2.89 (br d, J=7.09 Hz, 2H), 2.95 (s, 2H), 2.91-3.01 (m, 2H), 5.54 (br s, 2H), 6.66-6.74 (m, 1H), 6.91 (d, J=7.46 Hz, 1H), 6.96 (d, J=7.34 Hz, 1H), 7.05-7.19 (m, 2H), 7.56 (br t, J=7.40 Hz, 1H), 8.51 (d, J=4.65 Hz, 1H); ESIMS found for C₂₆H₂₉N₃O₂S m/z 448.1 (M+1).

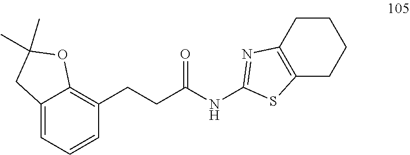 [SMILES: CC1(C)Cc2cccc(CCC(=O)Nc3nc4c(s3)CCCC4)c2O1]

3-(2,2-Dimethyl-2,3-dihydrobenzofuran-7-yl)-N-(4,5,6,7-tetrahydrobenzo[d]thiazol-2-yl)propanamide (105) was prepared in accordance with the procedures described in Schemes 24 and 8. 50% yield. ¹H NMR (400 MHz, CHLOROFORM-d) δ ppm 1.46 (s, 6H), 1.77-1.89 (m, 4H), 2.56-2.64 (m, 2H), 2.68 (br d, J=1.96 Hz, 2H), 2.69-2.77 (m, 2H), 2.96 (t, J=7.76 Hz, 2H), 2.99 (s, 2H), 6.70-6.78 (m, 1H), 6.94 (d, J=7.46 Hz, 1H), 7.00 (d, J=7.21 Hz, 1H), 8.96 (br s, 1H); ESIMS found for C₂₀H₂₄N₂O₂S: m/z 357.2 (M+1).

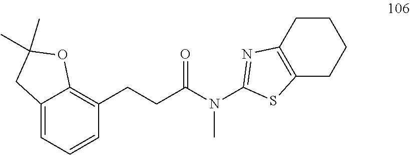 [SMILES: CN(C(=O)CCc1cccc2c1OC(C)(C)C2)c1nc2c(s1)CCCC2]

3-(2,2-Dimethyl-2,3-dihydrobenzofuran-7-yl)-N-methyl-N-(4,5,6,7-tetrahydrobenzo[d]thiazol-2-yl)propanamide (106) was prepared in accordance with the procedures described in Schemes 24 and 8. ¹H NMR (400 MHz, CHLOROFORM-d) δ ppm 1.45 (s, 6H), 1.78-1.90 (m, 4H), 2.68 (q, J=5.71 Hz, 4H), 2.91 (br d, J=7.83 Hz, 2H), 3.00 (s, 2H), 2.95-3.03 (m, 2H), 3.63 (br s, 3H), 6.71-6.78 (m, 1H), 6.95-7.04 (m, 2H); ESIMS found for C₂₁H₂₆N₂O₂S: m/z 371.1 (M+1).

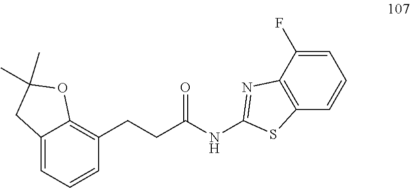 [SMILES: CC1(C)Cc2cccc(CCC(=O)Nc3nc4c(F)cccc4s3)c2O1]

3-(2,2-Dimethyl-2,3-dihydrobenzofuran-7-yl)-N-(4-fluorobenzo[d]thiazol-2-yl)propanamide (107) was prepared in accordance with the procedures described in Schemes 24 and 8. 3.6% yield. ¹H NMR (400 MHz, CHLOROFORM-d) δ ppm 1.46 (s, 6H), 2.79-2.86 (m, 2H), 2.97-3.05 (m, 4H), 6.71-6.79 (m, 1H), 6.94 (d, J=7.46 Hz, 1H), 7.01 (d, J=7.21 Hz, 1H), 7.09-7.17 (m, 1H), 7.24-7.28 (m, 1H), 7.55-7.61 (m, 1H), 9.57 (br s, 1H); ESIMS found for C₂₀H₁₉FN₂O₂S: m/z 371.3 (M+1).

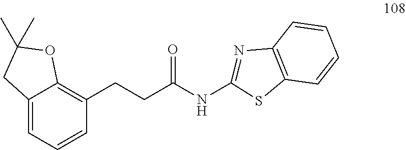 [SMILES: CC1(C)Cc2cccc(CCC(=O)Nc3nc4ccccc4s3)c2O1]

N-(Benzo[d]thiazol-2-yl)-3-(2,2-dimethyl-2,3-dihydrobenzofuran-7-yl) propanamide (108) was prepared in accordance with the procedures described in Schemes 24 and 8. 31% yield. ¹H NMR (400 MHz, CHLOROFORM-d) δ ppm 1.42 (s, 6H), 2.78-2.85 (m, 2H), 2.97 (s, 2H), 2.98-3.01 (m, 2H), 6.69-6.75 (m, 1H), 6.91 (d, J=7.58 Hz, 1H), 7.00 (br d, J=7.09 Hz, 1H), 7.27-7.33 (m, 1H), 7.35-7.43 (m, 1H), 7.72 (d, J=8.07 Hz, 1H), 7.81 (d, J=7.95 Hz, 1H), 10.09 (br s, 1H); ESIMS found for C₂₀H₂₀N₂O₂S: m/z 353.3 (M+1).

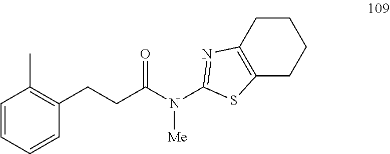 [SMILES: Cc1ccccc1CCC(=O)N(C)c1nc2c(s1)CCCC2]

N-Methyl-N-(4,5,6,7-tetrahydrobenzo[d]thiazol-2-yl)-3-(o-tolyl) propanamide (109) was prepared in accordance with the procedures described in Scheme 8 from 3-(o-tolyl)propanoic acid. ESIMS found for C₁₂H₁₈N₂OS: m/z 315.1 (M+1).

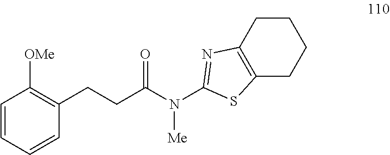 [SMILES: COc1ccccc1CCC(=O)N(C)c1nc2c(s1)CCCC2]

3-(2-Methoxyphenyl)-N-methyl-N-(4,5,6,7-tetrahydrobenzo[d]thiazol-2-yl) propanamide (110) was prepared in accordance with the procedures described in Scheme 8 from 3-(2-methoxyphenyl)propanoic acid. ESIMS found for C₁₈H₂₂N₂O₂: m/z 331.2 (M+1).

2-((1H-Indol-4-yl)oxy)-N-methyl-N-(4,5,6,7-tetrahydrobenzo[d]thiazol-2-yl) acetamide (111) is depicted below in Scheme 25.

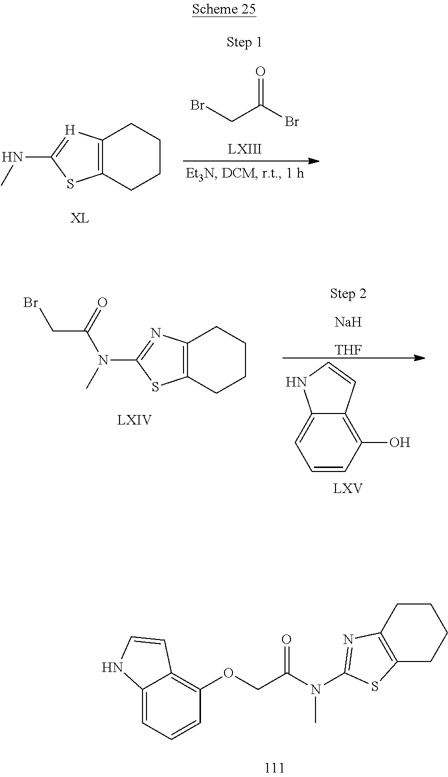

### Step 1

To a solution of N-methyl-4,5,6,7-tetrahydrobenzo[d]thiazol-2-amine (XL) (9.20 g, 54.8 mmol) in anhydrous DCM (15 mL) cooled to 0° C. was added Et₃N (8.31 g, 82.1 mmol) and 2-bromoacetyl bromide (LXIII) (16.6 g, 82.1 mmol) dropwise simultaneously. After 1.5 h, solvent was removed under reduced pressure and cold water (100 mL) was added. The mixture was extracted with EtOAc (2×100 mL). The organic layer was washed with brine (100 mL), dried over Na₂SO₄, filtered, and concentrated under reduced pressure. Flash chromatography (EtOAc:hexanes, gradient elution) afforded 10 g (63%) of 2-bromo-N-methyl-N-(4,5,6,7-tetrahydrobenzo[d]thiazol-2-yl)acetamide (LXIV), a pale yellow solid. ESIMS found for C₁₀H₁₃BrN₂OS: m/z 288.8 (M+1).

### Step 2

To a suspension of NaH (60% in oil, 22.5 mg, 0.563 mmol) in anhydrous THE (5 mL) cooled to 0° C. was added 1H-indol-4-ol (LXV) (75 mg, 0.563 mmol). After stirring for 20 min, 2-bromo-N-methyl-N-(4,5,6,7-tetrahydrobenzo[d]thiazol-2-yl)acetamide (LXIV) (163 mg, 0.563 mmol) was added and the reaction mixture was allowed to warm to rt over 30 min. After 2 h, a satd. aqueous NH₄Cl solution (30 mL) was added, and the mixture was extracted with EtOAc (80 mL). The organic layer was dried over MgSO₄, filtered, and concentrated under reduced pressure. Flash chromatography (EtOAc:hexanes, gradient elution) afforded 97 mg (60%) of 2-((1H-indol-4-yl)oxy)-N-methyl-N-(4,5,6,7-tetrahydrobenzo[d]thiazol-2-yl)acetamide (111), a white solid. ESIMS found for C₁₈H₁₉N₃O₂S: m/z 342.2 (M+1). ¹H NMR (400 MHz, CHLOROFORM-d) δ ppm 1.84 (br d, J=4.52 Hz, 4H), 2.62-2.74 (m, 4H), 3.76 (br s, 3H), 5.07 (s, 2H), 6.53 (br s, 1H), 6.68 (br s, 1H), 7.03-7.10 (m, 2H), 7.12 (br s, 1H), 8.23 (br s, 1H); ESIMS found for C₁₈H₁₉N₃O₂S: m/z 342.2 (M+1).

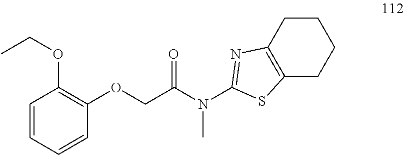 [SMILES: CCOc1ccccc1OCC(=O)N(C)c1nc2c(s1)CCCC2]

2-(2-Ethoxyphenoxy)-N-methyl-N-(4,5,6,7-tetrahydrobenzo[d]thiazol-2-yl) acetamide (112) was prepared in accordance with the procedures described in Scheme 25. ¹H NMR (400 MHz, CHLOROFORM-d) δ ppm 1.43 (br t, J=6.97 Hz, 3H), 1.84 (br d, J=2.81 Hz, 4H), 2.63-2.73 (m, 4H), 3.74 (br s, 3H), 4.09 (q, J=6.93 Hz, 2H), 4.98 (s, 2H), 6.82-6.93 (m, 2H), 6.96 (br d, J=7.21 Hz, 2H); ESIMS found for C₁₈H₂₂N₂O₃S: m/z 347.4 (M+1).

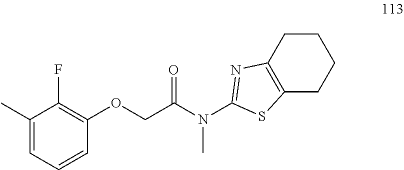 [SMILES: Cc1cccc(OCC(=O)N(C)c2nc3c(s2)CCCC3)c1F]

2-(2-Fluoro-3-methylphenoxy)-N-methyl-N-(4,5,6,7-tetrahydrobenzo[d]thiazol-2-yl)acetamide (113) was prepared in accordance with the procedures described in Scheme 25. ¹H NMR (400 MHz, CHLOROFORM-d) δ ppm 1.84 (br d, J=4.52 Hz, 4H), 2.27 (d, J=1.71 Hz, 3H), 2.68 (br d, J=4.28 Hz, 4H), 3.71 (br s, 3H), 4.99 (s, 2H), 6.77-6.87 (m, 2H), 6.88-6.95 (m, 1H); ESIMS found for C₁₇H₁₉FN₂O₂S: m/z 335.4 (M+1).

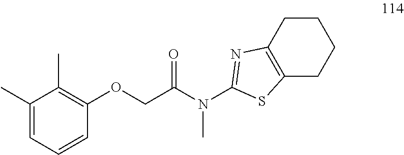 [SMILES: Cc1cccc(OCC(=O)N(C)c2nc3c(s2)CCCC3)c1C]

2-(2,3-Dimethylphenoxy)-N-methyl-N-(4,5,6,7-tetrahydrobenzo[d]thiazol-2-yl)acetamide (114) was prepared in accordance with the procedures described in Scheme 25. ¹H NMR (400 MHz, CHLOROFORM-d) δ ppm 1.85 (br d, J=4.40 Hz, 4 H), 2.20 (s, 3H), 2.27 (s, 3H), 2.64-2.74 (m, 4H), 3.71 (br s, 3H), 4.91 (s, 2H), 6.68 (br d, J=7.83 Hz, 1H), 6.82 (br d, J=7.46 Hz, 1H), 7.02 (t, J=7.82 Hz, 1H); ESIMS found for C₁₈H₂₂N₂O₂S: m/z 331.2 (M+1).

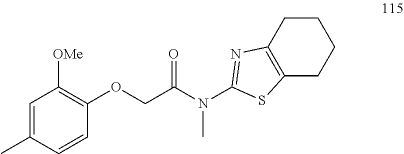 [SMILES: COc1cc(C)ccc1OCC(=O)N(C)c1nc2c(s1)CCCC2]

2-(2-Methoxy-4-methylphenoxy)-N-methyl-N-(4,5,6,7-tetrahydrobenzo[d]thiazol-2-yl)acetamide (115) was prepared in accordance with the procedures described in Scheme 25. ¹H NMR (400 MHz, CHLOROFORM-d) δ ppm 1.84 (br d, J=3.18 Hz, 4H), 2.29 (s, 3H), 2.68 (br d, J=4.40 Hz, 4H), 3.71 (br s, 3H), 3.84 (s, 3H), 4.94 (s, 2H), 6.66 (br d, J=8.07 Hz, 1H), 6.72 (s, 1H), 6.83 (br d, J=7.82 Hz, 1H); ESIMS found for C₁₈H₂₂N₂O₃S: m/z 347.0 (M+1).

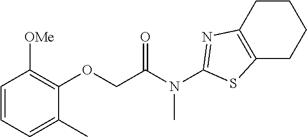 [SMILES: COc1cccc(C)c1OCC(=O)N(C)c1nc2c(s1)CCCC2]

2-(2-Methoxy-6-methylphenoxy)-N-methyl-N-(4,5,6,7-tetrahydrobenzo[d]thiazol-2-yl)acetamide (116) was prepared in accordance with the procedures described in Scheme 25. ¹H NMR (400 MHz, CHLOROFORM-d) δ ppm 1.85 (br d, J=2.81 Hz, 4H), 2.30 (s, 3H), 2.70 (br s, 4H), 3.73 (br s, 3H), 3.79 (s, 3H), 4.85 (s, 2H), 6.76 (br t, J=7.03 Hz, 2H), 6.93-7.01 (m, 1H); ESIMS found for C₁₈H₂₂N₂O₃S: m/z 347.4 (M+1).

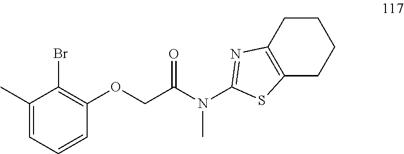 [SMILES: Cc1cccc(OCC(=O)N(C)c2nc3c(s2)CCCC3)c1Br]

2-(2-Bromo-3-methylphenoxy)-N-methyl-N-(4,5,6,7-tetrahydrobenzo[d]thiazol-2-yl)acetamide (117) was prepared in accordance with the procedures described in Scheme 25. ¹H NMR (400 MHz, CHLOROFORM-d) δ ppm 1.85 (br s, 4H), 2.41 (s, 3H), 2.68 (br d, J=4.28 Hz, 4H), 3.76 (br s, 3H), 5.00 (br s, 2H), 6.76 (br d, J=6.72 Hz, 1H), 6.90 (br d, J=7.46 Hz, 1H), 7.12 (t, J=7.89 Hz, 1H); ESIMS found for C₁₇H₁₉⁸¹BrN₂O₂S: m/z 397.2 (M+1).

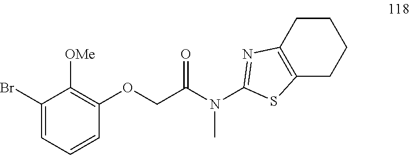 [SMILES: COc1c(Br)cccc1OCC(=O)N(C)c1nc2c(s1)CCCC2]

2-(3-Bromo-2-methoxyphenoxy)-N-methyl-N-(4,5,6,7-tetrahydrobenzo[d]thiazol-2-yl)acetamide (118) was prepared in accordance with the procedures described in Scheme 25. ¹H NMR (400 MHz, CHLOROFORM-d) δ ppm 1.79-1.91 (m, 4H), 2.65-2.74 (m, 4H), 3.69 (br s, 3H), 3.92 (s, 3H), 5.01 (s, 2H), 6.85-6.93 (m, 2H), 7.16-7.23 (m, 1H); ESIMS found for C₁₇H₁₉BrN₂O₃S: m/z 411.2 (M+1).

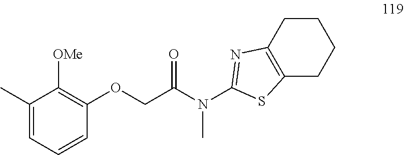 [SMILES: COc1c(C)cccc1OCC(=O)N(C)c1nc2c(s1)CCCC2]

2-(2-Methoxy-3-methylphenoxy)-N-methyl-N-(4,5,6,7-tetrahydrobenzo[d]thiazol-2-yl)acetamide (119) was prepared in accordance with the procedures described in Scheme 14 from compound 118 and trimethylboroxine. ¹H NMR (400 MHz, CHLOROFORM-d) δ ppm 1.78-1.90 (m, 4H), 2.27 (s, 3H), 2.69 (q, J=5.42 Hz, 4H), 3.70 (br s, 2H), 3.86 (s, 3H), 4.97 (s, 2H), 6.77 (br d, J=7.21 Hz, 1H), 6.80-6.86 (m, 1H), 6.88-6.95 (m, 1H); ESIMS found for C₁₈H₂₂N₂O₃S: m/z 347.2 (M+1).

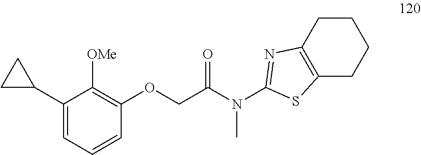 [SMILES: COc1c(OCC(=O)N(C)c2nc3c(s2)CCCC3)cccc1C1CC1]

2-(3-Cyclopropyl-2-methoxyphenoxy)-N-methyl-N-(4,5,6,7-tetrahydrobenzo [d]thiazol-2-yl)acetamide (120) was prepared in accordance with the procedures described in Scheme 14 from compound 118 and cyclopropylboronic acid. ¹H NMR (400 MHz, CHLOROFORM-d) δ ppm 6.91 (t, J=8 Hz, 1H), 6.74 (broad d, J=8 Hz, 1H), 6.46 (d, J=8 Hz, 1H), 4.98 (s, 2H), 3.91 (s, 3H), 3.71 (broad s, 3H), 2.73-2.65 (m, 4H), 2.27-2.18 (m, 1H), 1.90-1.80 (m 4H), 1.00-0.93 (m, 2H), 0.69-0.63 (m, 2H); ESIMS found for C_{1z}H_{2z}N_{z}O_{z}S: m/z 373.1 (M+1).

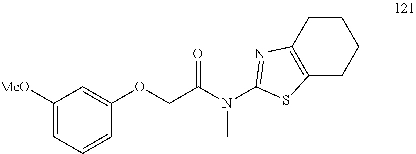 [SMILES: COc1cccc(OCC(=O)N(C)c2nc3c(s2)CCCC3)c1]

2-(3-Methoxyphenoxy)-N-methyl-N-(4,5,6,7-tetrahydrobenzo[d]thiazol-2-yl) acetamide (121) was prepared in accordance with the procedures described in Scheme 25. ESIMS found for C₁₇H₂₀N₂O₃S: m/z 333.2 (M+1).

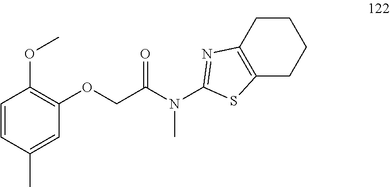 [SMILES: COc1ccc(C)cc1OCC(=O)N(C)c1nc2c(s1)CCCC2]

2-(2-Methoxy-5-methylphenoxy)-N-methyl-N-(4,5,6,7-tetrahydrobenzo[d]thiazol-2-yl)acetamide (122) was prepared in accordance with the procedures described in Scheme 25. ESIMS found for C₁₈H₂₂N₂O₃S: m/z 347.4 (M+1).

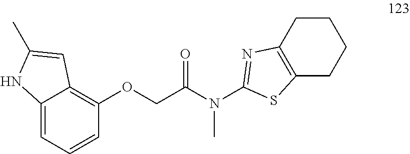 [SMILES: Cc1cc2c(OCC(=O)N(C)c3nc4c(s3)CCCC4)cccc2[nH]1]

N-Methyl-2-((2-methyl-1H-indol-4-yl)oxy)-N-(4,5,6,7-tetrahydrobenzo[d]thiazol-2-yl)acetamide (123) was prepared in accordance with the procedures described in Scheme 25. ¹H NMR (400 MHz, CHLOROFORM-d) δ ppm 7.91 (broad s, 1H), 7.02-6.91 (m, 2H), 6.53-6.45 (m, 1H), 6.34 (broad s, 1H), 5.04 (s, 2H), 3.75 (broad s, 3H), 2.71-2.60 (m, 4H), 2.42 (s, 3H), 1.90-1.80 (m, 4H); ESIMS found for C₁₉H₂₁N₃O₂S: m/z 356.0 (M+1).

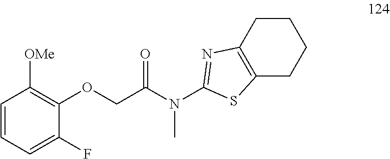 [SMILES: COc1cccc(F)c1OCC(=O)N(C)c1nc2c(s1)CCCC2]

2-(2-Fluoro-6-methoxyphenoxy)-N-methyl-N-(4,5,6,7-tetrahydrobenzo[d]thiazol-2-yl)acetamide (124) was prepared in accordance with the procedures described in Scheme 25. ESIMS found for C₁₇H₁₉FN₂O₃S: m/z 351.3 (M+1).

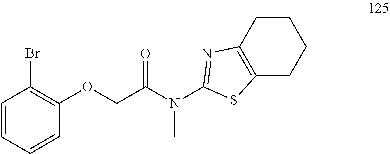 [SMILES: CN(C(=O)COc1ccccc1Br)c1nc2c(s1)CCCC2]

N-Methyl-N-(4,5,6,7-tetrahydrobenzo[d]thiazol-2-yl)-2-(o-tolyloxy) acetamide (125) was prepared in accordance with the procedures described in Scheme 25. ESIMS found for C₁₇H₂₀N₂O₂S: m/z 317.2 (M+1).

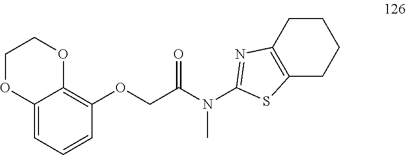 [SMILES: CN(C(=O)COc1cccc2c1OCCO2)c1nc2c(s1)CCCC2]

2-((2,3-Dihydrobenzo[b][1,4]dioxin-5-yl)oxy)-N-methyl-N-(4,5,6,7-tetrahydrobenzo[d]thiazol-2-yl)acetamide (126) was prepared in accordance with the procedures described in Scheme 25. ESIMS found for C₁₈H₂₀N₂O₄S: m/z 361.2 (M+1).

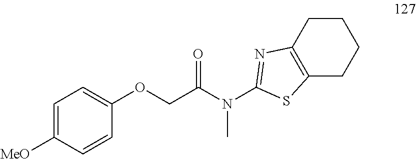 [SMILES: COc1ccc(OCC(=O)N(C)c2nc3c(s2)CCCC3)cc1]

2-(4-Methoxyphenoxy)-N-methyl-N-(4,5,6,7-tetrahydrobenzo[d]thiazol-2-yl) acetamide (127) was prepared in accordance with the procedures described in Scheme 25. ESIMS found for C₁₇H₂₀N₂O₃S: m/z 333.4 (M+1).

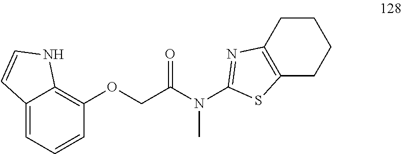 [SMILES: CN(C(=O)COc1cccc2cc[nH]c12)c1nc2c(s1)CCCC2]

2-((1H-Indol-7-yl)oxy)-N-methyl-N-(4,5,6,7-tetrahydrobenzo[d]thiazol-2-yl) acetamide (128) was prepared in accordance with the procedures described in Scheme 25. ESIMS found for C₁₇H₂₀N₂O₃S: m/z 342.4 (M+1).

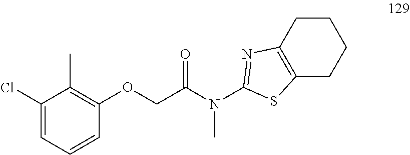 [SMILES: Cc1c(Cl)cccc1OCC(=O)N(C)c1nc2c(s1)CCCC2]

2-(3-Chloro-2-methylphenoxy)-N-methyl-N-(4,5,6,7-tetrahydrobenzo[d]thiazol-2-yl)acetamide (129) was prepared in accordance with the procedures described in Scheme 25. ESIMS found for C₁₇H₁₉ClN₂O₂S: m/z 351.4 (M+1).

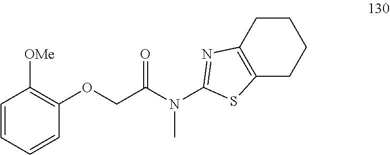 [SMILES: COc1ccccc1OCC(=O)N(C)c1nc2c(s1)CCCC2]

2-(2-Methoxyphenoxy)-N-methyl-N-(4,5,6,7-tetrahydrobenzo[d]thiazol-2-yl) acetamide (130) was prepared in accordance with the procedures described in Scheme 25. ESIMS found for C₁₇H₂₀N₂O₃S: m/z 333.4 (M+1).

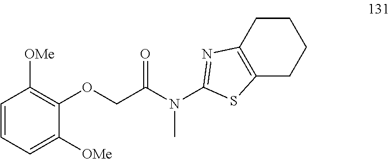 [SMILES: COc1cccc(OC)c1OCC(=O)N(C)c1nc2c(s1)CCCC2]

2-(2,6-Dimethoxyphenoxy)-N-methyl-N-(4,5,6,7-tetrahydrobenzo[d]thiazol-2-yl)acetamide (131) was prepared in accordance with the procedures described in Scheme 25. ESIMS found for C₁₈H₂₂N₂O₄S: m/z 363.3 (M+1).

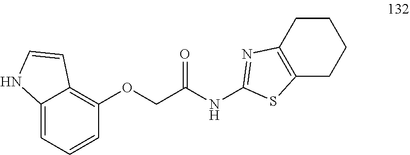 [SMILES: O=C(COc1cccc2[nH]ccc12)Nc1nc2c(s1)CCCC2]

2-((1H-Indol-4-yl)oxy)-N-(4,5,6,7-tetrahydrobenzo[d]thiazol-2-yl)acetamide (132) was prepared in accordance with the procedures described in Scheme 25. 1H NMR (400 MHz, CHLOROFORM-d) δ 9.60 (broad s, 1H), 8.27 (broad s, 1H), 7.20-7.18 (m, 1H), 7.15-7.07 (m, 2H), 6.75-6.72 (m, 1H), 6.53 (dd, J=8, 4 Hz, 1H), 4.86 (s, 2H), 2.75-2.60 (m, 4H), 1.91-1.81 (4H); ESIMS found for C₁₇H₁₇N₃O₂S: m/z 328.3 (M+1).

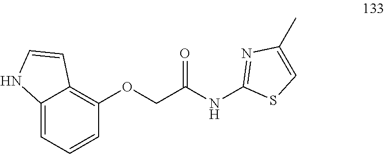 [SMILES: Cc1csc(NC(=O)COc2cccc3[nH]ccc23)n1]

2-((1H-Indol-4-yl)oxy)-N-(4-methylthiazol-2-yl)acetamide (133) was prepared in accordance with the procedures described in Scheme 25. ¹H NMR (400 MHz, CHLOROFORM-d) δ ppm 2.36 (d, J=0.86 Hz, 3H), 4.87 (s, 2H), 6.53 (dd, J=6.60, 1.83 Hz, 1H), 6.57 (d, J=0.98 Hz, 1H), 6.73 (t, J=2.45 Hz, 1H), 7.08-7.15 (m, 2H), 7.17-7.21 (m, 1H), 8.32 (br s, 1H), 9.75 (br s, 1H); ESIMS found for C₁₄H₁₃N₃O₂S: m/z 288.1 (M+1).

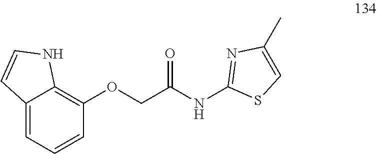 [SMILES: Cc1csc(NC(=O)COc2cccc3cc[nH]c23)n1]

2-((1H-Indol-7-yl)oxy)-N-(4-methylthiazol-2-yl)acetamide (134) was prepared in accordance with the procedures described in Scheme 25. ¹H NMR (400 MHz, CHLOROFORM-d) δ ppm 2.36 (d, J=0.86 Hz, 3H), 4.86 (s, 2H), 6.57 (dd, J=2.93, 2.20 Hz, 1H), 6.58 (d, J=0.98 Hz, 1H), 6.63 (d, J=7.70 Hz, 1H), 7.03 (t, J=7.89 Hz, 1H), 7.35 (d, J=7.95 Hz, 1H), 9.59 (br s, 1H), 10.35 (br s, 1H); ESIMS found for C₁₄H₁₃N₃O₂S: m/z 288.1 (M+1).

The synthesis of 2-(Indolin-4-yloxy)-N-(4,5,6,7-tetrahydrobenzo[d]thiazol-2-yl)acetamide (135) is depicted below in Scheme 26.

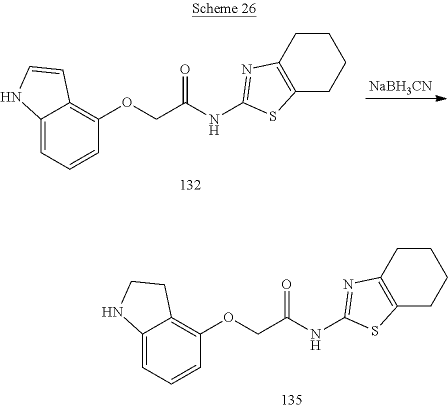

To a mixture of 2-((1H-indol-4-yl)oxy)-N-(4,5,6,7-tetrahydrobenzo[d] thiazol-2-yl)acetamide (132) (20 mg, 0.061 mmol) in HOAc (2 mL) was added NaBH₃CN (9.6 mg, 0.15 mmol). After 18 h, the mixture was diluted with water (30 mL) and basified with 1 M aq. NaOH to pH 9-10. The solution was extracted with EtOAc (2×30 mL) and the combined organic layers were washed sequentially with water (30 mL), and brine (30 mL). The organic layer was dried (MgSO₄), filtered and concentrated under reduced pressure. Purifications by column chromatography (EtOAc:hexanes, gradient elution) followed by column chromatography (EtOAc:hexanes with 0.1% Et₃N, gradient elution) afforded 2-(indolin-4-yloxy)-N-(4,5,6,7-tetrahydrobenzo[d]thiazol-2-yl)acetamide (135) as an off-white solid (10 mg, 0.035 mmol, 57%). ¹H NMR (400 MHz, CHLOROFORM-d) δ ppm 9.41 (broad s, 1H), 6.98 (t, J=8 Hz, 1H), 6.38 (d, J=8 Hz, 1H), 6.22 (d, J=8 Hz, 1H), 4.70 (s, 2H), 3.62 (t, J=8 Hz, 2H), 3.95-3.75 (broad s, 1H), 3.09 (t, J=8 Hz, 2H), 2.74-2.61 (m, 4H), 1.90-1.80 (m, 4H); ESIMS found for C₁₇H₁₉N₃O₂S: m/z 330.3 (M+1).

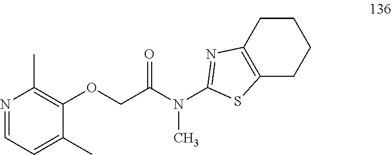 [SMILES: Cc1ccnc(C)c1OCC(=O)N(C)c1nc2c(s1)CCCC2]

2-((2,4-Dimethylpyridin-3-yl)oxy)-N-methyl-N-(4,5,6,7-tetrahydrobenzo[d]thiazol-2-yl)acetamide (136) was prepared in accordance with the procedures described in Scheme 25. ESIMS found for C₁₇H₂₁N₃O₂S: m/z 332.3 (M+1).

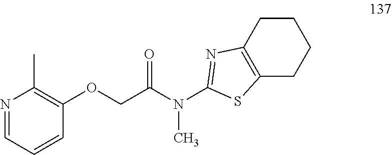 [SMILES: Cc1ncccc1OCC(=O)N(C)c1nc2c(s1)CCCC2]

N-Methyl-2-((2-methylpyridin-3-yl)oxy)-N-(4,5,6,7-tetrahydrobenzo[d]thiazol-2-yl)acetamide (137) was prepared in accordance with the procedures described in Scheme 25. ESIMS found for C₁₆H₁₉N₃O₂S: m/z 318.1 (M+1).

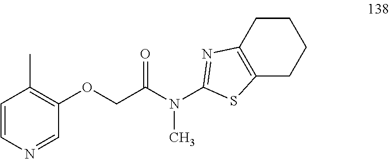 [SMILES: Cc1ccncc1OCC(=O)N(C)c1nc2c(s1)CCCC2]

N-Methyl-2-((4-methylpyridin-3-yl)oxy)-N-(4,5,6,7-tetrahydrobenzo[d]thiazol-2-yl)acetamide (138) was prepared in accordance with the procedures described in Scheme 25. ESIMS found for C₁₆H₁₉N₃O₂S: m/z 318.2 (M+1).

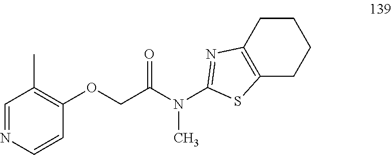 [SMILES: Cc1cnccc1OCC(=O)N(C)c1nc2c(s1)CCCC2]

N-Methyl-2-((3-methylpyridin-4-yl)oxy)-N-(4,5,6,7-tetrahydrobenzo[d]thiazol-2-yl)acetamide (139) was prepared in accordance with the procedures described in Scheme 25. ESIMS found for C₁₆H₁₉N₃O₂S: m/z 318.1 (M+1).

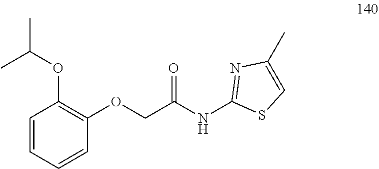 [SMILES: Cc1csc(NC(=O)COc2ccccc2OC(C)C)n1]

2-(2-isopropoxyphenoxy)-N-(4-methylthiazol-2-yl)acetamide (140) was prepared in accordance with the procedures described in Scheme 25. ESIMS found for C₁₅H₁₈N₂O₃S: m/z 307.4 (M+1).

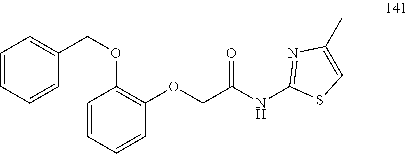 [SMILES: Cc1csc(NC(=O)COc2ccccc2OCc2ccccc2)n1]

2-(2-(Benzyloxy)phenoxy)-N-(4-methylthiazol-2-yl)acetamide (141) was prepared in accordance with the procedures described in Scheme 25. ¹H NMR (400 MHz, CHLOROFORM-d) δ ppm 2.34 (s, 3H), 4.72 (s, 2H), 5.20 (s, 2H), 6.53 (d, J=0.86 Hz, 1H), 6.89-6.96 (m, 1H), 6.97-7.07 (m, 3H), 7.29 (d, J=7.09 Hz, 1H), 7.31-7.38 (m, 2H), 7.46 (d, J=7.21 Hz, 2H), 10.18 (br s, 1H); ESIMS found for C₁₉H₁₈N₂O₃S: m/z 355.3 (M+1)₆

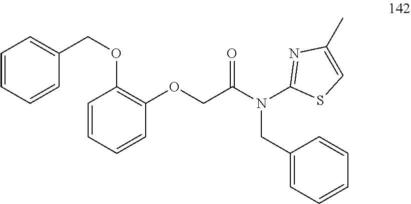 [SMILES: Cc1csc(N(Cc2ccccc2)C(=O)COc2ccccc2OCc2ccccc2)n1]

N-Benzyl-2-(2-(benzyloxy)phenoxy)-N-(4-methylthiazol-2-yl)acetamide (142) was prepared in accordance with the procedures described in Scheme 25. ¹H NMR (400 MHz, CHLOROFORM-d) δ ppm 2.32 (d, J=0.73 Hz, 3H), 4.88 (br s, 2H), 5.09 (s, 2H), 5.54 (br s, 2H), 6.60 (d, J=0.61 Hz, 1H), 6.80-6.86 (m, 2H), 6.91 (dt, J=3.03, 1.48 Hz, 2H), 7.17 (br d, J=6.72 Hz, 2H), 7.26-7.37 (m, 6H), 7.38-7.43 (m, 2H); ESIMS found for C₂₆H₂₄N₂O₃S: m/z 445.3 (M+1).

Preparation of N-(2-((2,2-dimethyl-2,3-dihydrobenzofuran-7-yl)oxy)ethyl) benzo[d]thiazol-2-amine (143) is depicted below in Scheme 27.

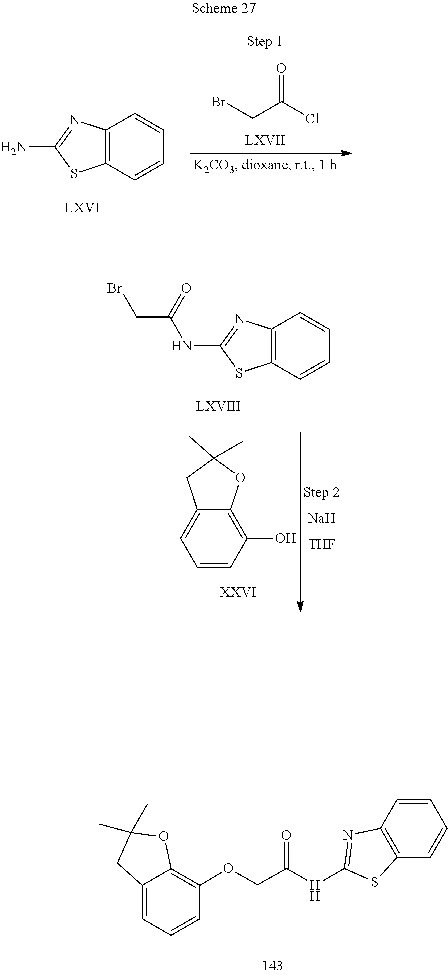

### Step 1

To a suspension of benzo[d]thiazol-2-amine (LXVI) (1.50 g, 10 mmol) in dry dioxane (10 mL) was added 2-bromoacetyl chloride (LXVII) (916 μL, 10 mmol) and K₂CO₃ (1.38, 10 mmol). The mixture was stirred at room temperature for 1 h. The reaction mixture was then poured into ice water. The precipitate was collected by filtration and the crude product was recrystallized from EtOH to provide N-(benzo[d]thiazol-2-yl)-2-bromoacetamide (LXVIII) as an off-white solid (1.1 g, 4.05 mmol, 40.5% yield).

### Step 2

To the solution of 2,2-dimethyl-2,3-dihydrobenzofuran-7-ol (XXVI)(182 mg, 1.12 mmol) in THF (3 mL) was added NaH (45 mg, 1.12 mmol) at 0° C. The mixture was stirred at room temperature for 20 min before adding N-(benzo[d]thiazol-2-yl)-2-bromoacetamide (LXVIII) (300 mg, 1.12 mmol) in THF (2 mL). The reaction mixture was stirred at room temperature for 2 h, then quenched by water, extracted with EtOAc, dried, and purified by column chromatography (EtOAc:hexanes, gradient elution) to give N-(benzo[d]thiazol-2-yl)-2-((2,2-dimethyl-2,3-dihydrobenzofuran-7-yl)oxy)acetamide (143) as a white solid (235 mg, 0.663 mmol, 59.2% yield). ¹H NMR (CDCl3, 500 MHz) δ ppm 1.54 (s, 6H), 3.05 (s, 2H), 4.82 (s, 2H), 6.8 (dt, J=14.8, 7.4 Hz, 2H), 6.90 (dd, J=6.9, 1.3 Hz, 1H), 7.33 (m, 1H), 7.45 (m, 1H), 7.83 (m, J=7.9 Hz, 2H); ESIMS found for C₁₉H₁₈N₂O₃S m/z 355.1 (M+H).

Preparation of N-(benzo[d]thiazol-2-yl)-2-((2,2-dimethyl-2,3-dihydrobenzofuran-7-yl)oxy)-N-methylacetamide (144) is depicted below in Scheme 28.

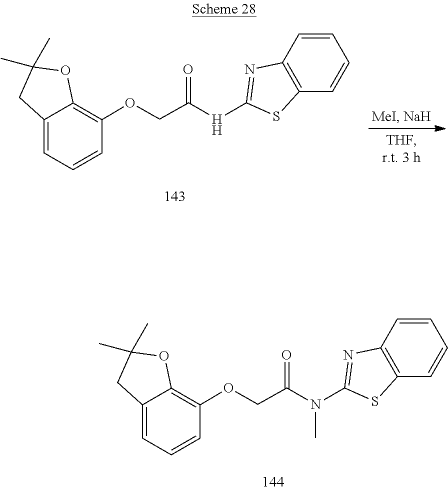

To the solution of N-(benzo[d]thiazol-2-yl)-2-((2,2-dimethyl-2,3-dihydrobenzofuran-7-yl)oxy)acetamide (143) (200 mg, 0.564 mmol) in THF (3 mL) was added NaH (25 mg, 0.62 mmol) at 0° C. The mixture was stirred at room temperature for 10 min, then Mel (39 μL, 0.62 mmol) was added. The reaction mixture was stirred at room temperature for 3 h, then ice water was added, and extracted with EtOAc. The organic layer was dried over anhydrous sodium sulfate, and purified by column chromatography (EtOAc:hexanes, gradient elution) to provide N-(benzo[d]thiazol-2-yl)-2-((2,2-dimethyl-2,3-dihydrobenzofuran-7-yl)oxy)-N-methylacetamide (144) as a white solid (135 mg, 0.366 mmol, 65.0% yield). ¹H NMR (CDCl₃, 500 MHz) δ ppm 1.52 (s, 3H), 1.58 (s, 3H), 3.03 (s, 2H), 3.82 (s, 3H), 4.96 (s, 2H), 6.71 (m, 3H), 7.31 (m, 2H), 7.46 (m, J=7.3, 1H), 7.65 (m, J=7.3, 2H); ESIMS found for C₂₀H₂₀N₂O₃S: m/z 369.0 (M+H).

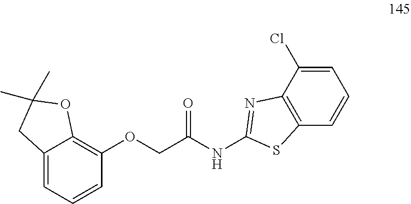 [SMILES: CC1(C)Cc2cccc(OCC(=O)Nc3nc4c(Cl)cccc4s3)c2O1]

N-(4-Chlorobenzo[d]thiazol-2-yl)-2-((2,2-dimethyl-2,3-dihydrobenzofuran-7-yl)oxy)acetamide (145) was prepared in accordance with the procedures described in Scheme 27. ¹H NMR (CDCl3, 500 MHz) δ ppm 1.56 (s, 6H), 3.05 (s, 2H), 4.84 (s, 2H), 6.80 (m, 2H), 6.90 (dd, J=7.09, 1.1 Hz, 1H), 7.26 (m, 1H), 7.47 (dd, J=7.88, 0.9 Hz, 1H), 7.73 (dd, J=7.88, 0.9 Hz, 1H); ESIMS found for C₁₉H₁₇ClN₂O₃S m/z 387.0 (M−H).

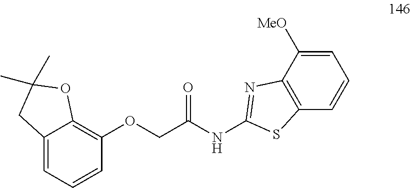 [SMILES: COc1cccc2sc(NC(=O)COc3cccc4c3OC(C)(C)C4)nc12]

2-((2,2-Dimethyl-2,3-dihydrobenzofuran-7-yl)oxy)-N-(4-methoxybenzo[d]thiazol-2-yl)acetamide (146) was prepared in accordance with the procedures described in Scheme 27. ¹H NMR (CDCl3, 500 MHz) δ ppm 1.52 (s, 6H), 3.03 (s, 2H), 4.02 (s, 2H), 4.81 (s, 2H), 6.76 (m, 2H), 6.88 (m, 2H), 7.28 (m, 1H), 7.42 (m, J=8.04 Hz, 2H); ESIMS found for C₂₀H₂₀N₂O₄S: m/z 383.0 (M−H).

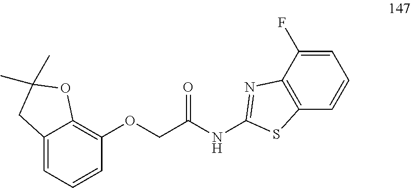 [SMILES: CC1(C)Cc2cccc(OCC(=O)Nc3nc4c(F)cccc4s3)c2O1]

2-((2,2-Dimethyl-2,3-dihydrobenzofuran-7-yl)oxy)-N-(4-fluorobenzo[d]thiazol-2-yl)acetamide (158) was prepared in accordance with the procedures described in Scheme 27. ¹H NMR (CDCl3, 500 MHz) δ ppm 1.47 (s, 6H), 2.98 (s, 2H), 4.76 (s, 2H), 6.73 (m, 2H), 6.83 (m, 1H), 7.10 (ddd, J=9.85, 8.59, 0.79 Hz, 1H), 7.22 (m, 1H), 7.53 (dd, J=7.88, 0.63 Hz, 1H), 10.24 (s, 1H); ESIMS found for C₁₉H₁₇FN₂O₃S: m/z 371.1 (M−H).

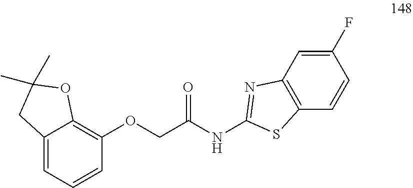 [SMILES: CC1(C)Cc2cccc(OCC(=O)Nc3nc4cc(F)ccc4s3)c2O1]

2-((2,2-Dimethyl-2,3-dihydrobenzofuran-7-yl)oxy)-N-(5-fluorobenzo[d]thiazol-2-yl)acetamide (148) was prepared in accordance with the procedures described in Scheme 27. ¹H NMR (CDCl3, 500 MHz) δ ppm 1.47 (s, 6H), 2.99 (s, 2H), 4.75 (s, 2H), 6.73 (m, 2H), 6.84 (d, J=5.99 Hz, 1H), 7.01 (m, 2H), 7.41 (d, J=8.51 Hz, 1H), 7.67 (m, 1H), 10.16 (s, 1H); ESIMS found for C₁₉H₁₇FN₂O₃S: m/z 323 (M+H).

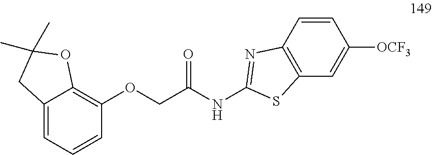 [SMILES: CC1(C)Cc2cccc(OCC(=O)Nc3nc4ccc(OC(F)(F)F)cc4s3)c2O1]

2-((2,2-Dimethyl-2,3-dihydrobenzofuran-7-yl)oxy)-N-(6-(trifluoromethoxy) benzo[d]thiazol-2-yl)acetamide (149) was prepared in accordance with the procedures described in Scheme 27. ¹H NMR (CDCl3, 500 MHz) δ ppm 1.54 (s, 6H), 3.06 (s, 2H), 4.03 (s, 2H), 4.83 (s, 2H), 6.79 (m, 2H), 6.91 (m, 2H), 7.32 (m, 1H), 7.67 (m, 1H), 7.80 (m, 1H); ESIMS found for C₂₀H₁₇F₃N₂O₄S: m/z 437.0 (M−H).

Preparation of 2-((2,2-dimethyl-2,3-dihydrobenzofuran-7-yl)oxy)-N-(4-(pyrrolidine-1-carbonyl)thiazol-2-yl)acetamide (150) is depicted below in Scheme 29.

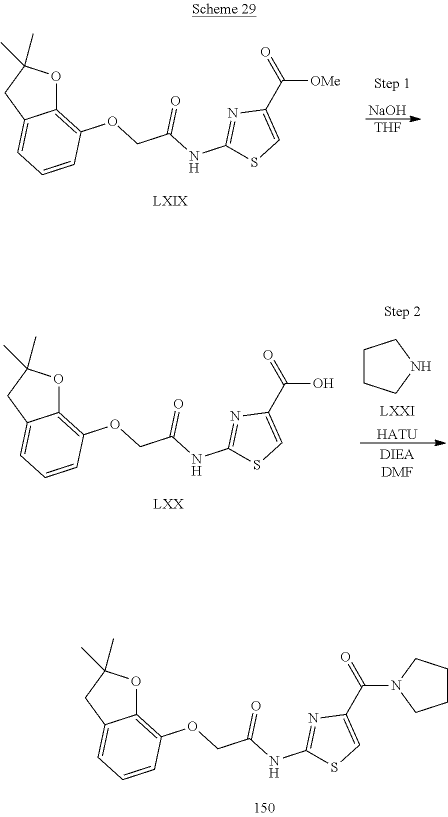

Methyl 2-(2-((2,2-dimethyl-2,3-dihydrobenzofuran-7-yl)oxy)acetamido) thiazole-4-carboxylate (LXIX) was prepared in accordance with the procedures described in Scheme 27. ¹H-NMR (500 MHz, CDCl3): δ 1.45 (s, 6H), 2.97 (s, 2H), 3.87 (s, 3H), 4.73 (s, 2H), 6.69 (m, 2H), 6.81 (dd, J=5.36, 3.15 Hz, 1H), 7.85 (s, 1H), 10.02 (s, 1H). ESIMS found for C₁₇H₁₈N₂O₅S: m/z 361.0 (M−1).

### Step 1

A mixture of LXIX (0.11 g, 0.30 mmol) and NaOH (60 mg, 1.5 mmol) in THE (3 mL) was heated at 45° C. for 3 h. The mixture was cooled and diluted with water, and acidified to pH 1 with 6 N HCl. The precipitate was filtered and washed with water, and dried to provide 90 mg (86% yield) of 2-(2-((2,2-dimethyl-2,3-dihydrobenzofuran-7-yl)oxy)acetamido) thiazole-4-carboxylic acid (LXX). ¹H NMR (500 MHz, CDCl3): δ 1.40 (s, 6H), 2.99 (s, 2H), 4.83 (s, 2H), 6.72 (m, 2H), 6.82 (d, J=6.94 Hz, 2H), 7.99 (s, 1H).

### Step 2

2-((2,2-Dimethyl-2,3-dihydrobenzofuran-7-yl)oxy)-N-(4-(pyrrolidine-1-carbonyl)thiazol-2-yl)acetamide (150) was prepared in accordance with the procedures described in Scheme 8 from pyrrolidine (LXXI). 62% yield. ¹H NMR (500 MHz, CDCl3): δ 1.44 (s, 6H), 1.86 (m, 4H), 2.98 (s, 2H), 3.59 (t, J=6.46 Hz, 2H), 3.77 (t, J=6.31 Hz, 2H), 4.73 (s, 2H), 6.73 (m, 2H), 6.83 (d, J=6.94 Hz, 2H), 7.57 (s, 1H), 9.99 (s, 1H).

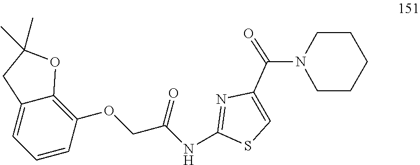 [SMILES: CC1(C)Cc2cccc(OCC(=O)Nc3nc(C(=O)N4CCCCC4)cs3)c2O1]

2-((2,2-Dimethyl-2,3-dihydrobenzofuran-7-yl)oxy)-N-(4-(piperidine-1-carbonyl)thiazol-2-yl)acetamide (151) was prepared in accordance with the procedures described in Scheme 29. ¹H NMR (500 MHz, CDCl3): δ 1.45 (s, 6H), 1.52 (s, 3H), 1.61 (s, 3H), 2.98 (s, 2H), 3.61 (s, 4H), 4.73 (s, 2H), 6.72 (m, 2H), 6.83 (d, J=6.62 Hz, 2H), 7.34 (s, 1H), 9.98 (s, 1H).

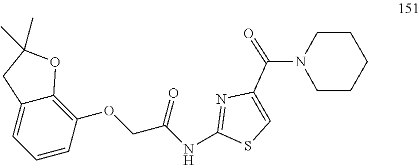 [SMILES: CC1(C)Cc2cccc(OCC(=O)Nc3nc(C(=O)N4CCCCC4)cs3)c2O1]

(S)-2-((2,2-Dimethyl-2,3-dihydrobenzofuran-7-yl)oxy)-N-(4-(3-(hydroxymethyl)pyrrolidine-1-carbonyl)thiazol-2-yl)acetamide (152) was prepared in accordance with the procedures described in Scheme 29. ¹H-NMR (500 MHz, CDCl₃): δ 1.44 (s, 6H), 1.75 (m, 1H), 2.0 (m, 1H), 2.41 (m, 1H), 2.98 (s, 2H), 3.59 (m, 3H), 3.75 (m, 1H), 3.93 (m, 1H), 4.73 (s, 2H), 6.73 (m, 2H), 6.82 (d, J=6.94 Hz, 2H), 7.59 (s, 1H), 10.15 (s, 1H).

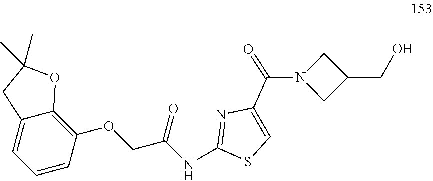 [SMILES: CC1(C)Cc2cccc(OCC(=O)Nc3nc(C(=O)N4CC(CO)C4)cs3)c2O1]

2-((2,2-Dimethyl-2,3-dihydrobenzofuran-7-yl)oxy)-N-(4-(3-(hydroxymethyl) azetidine-1-carbonyl)thiazol-2-yl)acetamide (153) was prepared in accordance with the procedures described in Scheme 29 from azetidin-3-ylmethanol HCl salt. 56% yield. ¹H-NMR (500 MHz, CDCl₃): δ 1.43 (s, 6H), 2.78 (m, 1H), 2.98 (s, 2H), 3.77 (m, 2H), 3.87 (dd, J=10.25 5.20 Hz, 1H), 4.16 (m, 1H), 4.31 (dd, J=9.77, 5.04 Hz, 1H), 4.54 (m, 1H), 4.72 (s, 2H), 6.73 (m, 2H), 6.84 (d, J=6.94 Hz, 1H), 7.67 (s, 1H), 9.98 (s, 1H)

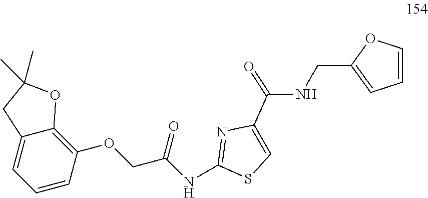 [SMILES: CC1(C)Cc2cccc(OCC(=O)Nc3nc(C(=O)NCc4ccco4)cs3)c2O1]

2-(2-((2,2-Dimethyl-2,3-dihydrobenzofuran-7-yl)oxy)acetamido)-N-(furan-2-ylmethyl)thiazole-4-carboxamide (154) was prepared in accordance with the procedures described in Scheme 29 from furan-2-ylmethanamine. 34% yield. ¹H-NMR (500 MHz, CDCl₃): δ 1.42 (s, 6H), 2.98 (s, 2H), 4.54 (d, J=5.68 Hz, 2H), 4.72 (s, 2H), 6.22 (s, 1H), 6.26 (s, 1H), 6.74 (m, 2H), 6.84 (d, J=6.94 Hz, 1H), 7.30 (s, 1H), 7.73 (s, 1H), 7.94 (s, 1H), 10.15 (s, 1H).

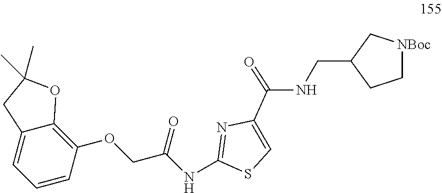 [SMILES: CC(C)(C)OC(=O)N1CCC(CNC(=O)c2csc(NC(=O)COc3cccc4c3OC(C)(C)C4)n2)C1]

tert-Butyl 3-((2-(2-((2,2-dimethyl-2,3-dihydrobenzofuran-7-yl)oxy) acetamido)thiazole-4-carboxamido)methyl)pyrrolidine-1-carboxylate (155) was prepared in accordance with the procedures described in Scheme 29. ¹H NMR (500 MHz, CDCl₃): δ 1.39 (s, 9H), 1.46 (s, 6H), 1.95 (m, 1H), 2.42 (m, 1H), 3.00 (m, 3H), 3.37 (m, 5H), 4.74 (s, 2H), 6.76 (m, 2H), 6.85 (d, J=6.94 Hz, 1H), 7.16 (s, 1H), 7.72 (s, 1H), 10.06 (s, 1H).

Preparation of 2-(2-((2,2-dimethyl-2,3-dihydrobenzofuran-7-yl)oxy) acetamido)-N-(pyrrolidin-3-ylmethyl)thiazole-4-carboxamide (156) is depicted below in Scheme 30.

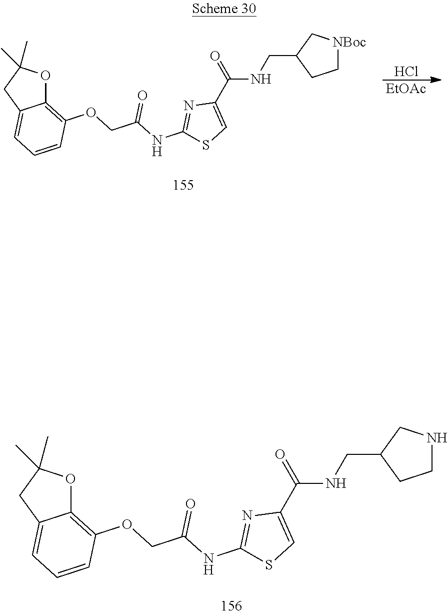 [SMILES: "EtOAc" ; CC(C)(C)OC(=O)N1CCC(CNC(=O)c2csc(NC(=O)COc3cccc4c3OC(C)(C)C4)n2)C1 ; Cl ; CC1(C)Cc2cccc(OCC(=O)Nc3nc(C(=O)NCC4CCNC4)cs3)c2O1]

A solution of compound 155 (40 mg, 0.075 mmol) in 1 mL EtOAc and 1N HCl (1 mL) was heated to 70° C. for 6 h. The solvents were removed under reduced pressure and the resultant residue triturated with ether and dried to afford 30 mg (93%) of 2-(2-((2,2-dimethyl-2,3-dihydrobenzofuran-7-yl)oxy)acetamido)-N-(pyrrolidin-3-ylmethyl)thiazole-4-carboxamide (156). ¹H-NMR (500 MHz, CDCl₃): δ 1.41 (s, 6H), 1.66 (m, 1H), 1.99 (m, 1H), 2.89 (s, 1H), 3.01 (s, 2H), 3.23 (m, 4H), 3.35 (m, 2H), 4.88 (s, 2H), 6.74 (m, 2H), 6.84 (d, J=6.94 Hz, 1H), 8.21 (t, J=6.15 Hz, 1H), 9.07 (s, 2H), 12.37 (s, 1H).

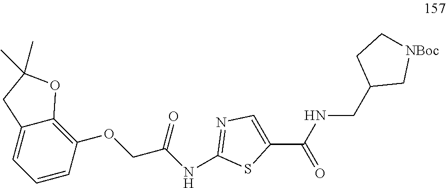 [SMILES: CC(C)(C)OC(=O)N1CCC(CNC(=O)c2cnc(NC(=O)COc3cccc4c3OC(C)(C)C4)s2)C1]

tert-Butyl 3-((2-(2-((2,2-dimethyl-2,3-dihydrobenzofuran-7-yl)oxy) acetamido)thiazole-5-carboxamido)methyl)pyrrolidine-1-carboxylate (157) was prepared in accordance with the procedures described in Scheme 29 from ethyl 2-(2-((2,2-dimethyl-2,3-dihydrobenzofuran-7-yl)oxy)acetamido)thiazole-5-carboxylate. ¹H NMR (500 MHz, CDCl₃): δ 1.39 (s, 9H), 1.45 (s, 6H), 1.94 (m, 1H), 2.44 (m, 1H), 2.98 (s, 2H), 3.46 (m, 6H), 4.74 (s, 2H), 5.90 (m, 1H), 6.73 (m, 2H), 6.83 (d, J=6.31 Hz, 1H), 7.83 (s, 1H), 10.22 (s, 1H).

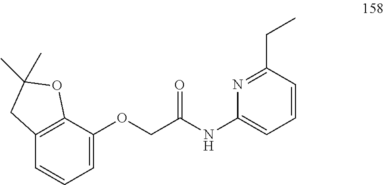 [SMILES: CCc1cccc(NC(=O)COc2cccc3c2OC(C)(C)C3)n1]

2-((2,2-Dimethyl-2,3-dihydrobenzofuran-7-yl)oxy)-N-(6-ethylpyridin-2-yl) acetamide (158) was prepared in accordance with the procedures described in Scheme 8. 10% yield. ¹H NMR (400 MHz, CHLOROFORM-d) δ ppm 1.28 (t, J=7.64 Hz, 3H), 1.53 (s, 6H), 2.73 (q, J=7.58 Hz, 2H), 3.05 (s, 2H), 4.71 (s, 2H), 6.77-6.86 (m, 2H), 6.88 (dd, J=7.09, 0.98 Hz, 1H), 6.94 (d, J=7.58 Hz, 1H), 7.63 (t, J=7.83 Hz, 1H), 8.06 (d, J=8.19 Hz, 1H), 9.05 (br s, 1H); ESIMS found for C₁₉H₂₂N₂O₃ m/z 327.5 (M+1).

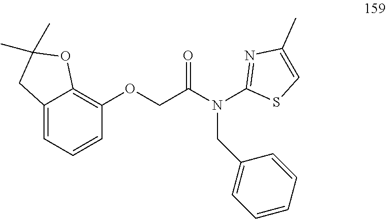 [SMILES: Cc1csc(N(Cc2ccccc2)C(=O)COc2cccc3c2OC(C)(C)C3)n1]

N-Benzyl-2-((2,2-dimethyl-2,3-dihydrobenzofuran-7-yl)oxy)-N-(4-methylthiazol-2-yl)acetamide (159) was prepared using the reductive amination method used in Scheme 10 and Step 2 used in Scheme 20. ESIMS found for C₂₃H₂₄N₂O₃S m/z 409.4 (M+1).

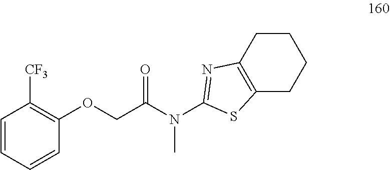 [SMILES: CN(C(=O)COc1ccccc1C(F)(F)F)c1nc2c(s1)CCCC2]

N-Methyl-N-(4,5,6,7-tetrahydrobenzo[d]thiazol-2-yl)-2-(2-(trifluoromethyl)phenoxy)acetamide (160) was prepared in accordance with the procedures described in Scheme 25. ¹H NMR (400 MHz, CHLOROFORM-d) δ ppm 7.63-7.57 (m, 1H), 7.47 (broad t, J=8 Hz, 1H), 7.12-6.97 (m, 2H), 5.05 (s, 2H), 3.74 (broad s, 3H), 2.74-2.64 (m, 4H), 1.91-1.78 (m, 4H); ESIMS found for C₁₇H₁₇F₃N₂O₂S: m/z 371.2 (M+1).

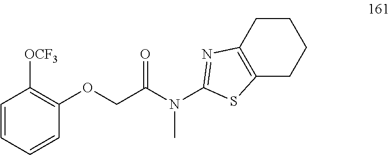 [SMILES: CN(C(=O)COc1ccccc1OC(F)(F)F)c1nc2c(s1)CCCC2]

N-Methyl-N-(4,5,6,7-tetrahydrobenzo[d]thiazol-2-yl)-2-(2-(trifluoromethoxy)phenoxy)acetamide (161) was prepared in accordance with the procedures described in Scheme 25. ¹H NMR (400 MHz, CHLOROFORM-d) δ ppm 7.28-7.19 (m, 2H), 7.09-6.96 (m, 2H), 5.01 (s, 2H), 3.72 (broad s, 3H), 2.73-2.64 (m, 4H), 1.90-1.80 (m, 4H); ESIMS found for C₁₇H₁₇F₃N₂O₃S: m/z 387.2 (M+1).

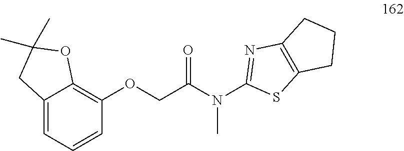 [SMILES: CN(C(=O)COc1cccc2c1OC(C)(C)C2)c1nc2c(s1)CCC2]

N-(5,6-Dihydro-4H-cyclopenta[d]thiazol-2-yl)-2-((2,2-dimethyl-2,3-dihydrobenzofuran-7-yl)oxy)-N-methylacetamide (162) was prepared in accordance with the procedures described in Schemes 12 and 8. ¹H NMR (400 MHz, CHLOROFORM-d) δ ppm 6.85-6.75 (m, 2H), 6.75-6.65 (m, 1H), 5.02 (s, 2H), 3.71 (broad s, 3H), 3.00 (S, 2H), 2.90-2.80 (m, 2H), 2.78-2.70 (m, 2H), 2.48-2.35 (m, 2H), 1.46 (s, 6H); ESIMS found for C₁₉H₂₂N₂O₃S: m/z 359.3 (M+1)

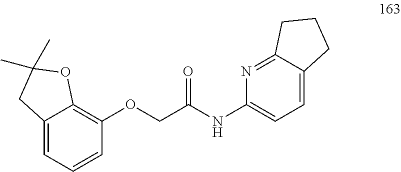 [SMILES: CC1(C)Cc2cccc(OCC(=O)Nc3ccc4c(n3)CCC4)c2O1]

N-(6,7-dihydro-5H-cyclopenta[b]pyridin-2-yl)-2-((2,2-dimethyl-2,3-dihydrobenzofuran-7-yl)oxy)acetamide (163) was prepared in accordance with the procedures described in Scheme 8. ¹H NMR (400 MHz, CHLOROFORM-d) δ ppm 9.02 (broad s, 1H), 8.03 (d, J=8 Hz, 1H), 7.53 (d, J=8 Hz, 1H), 6.86 (dd, J=8, 1 Hz, 1H), 6.85-6.70 (m, 2H), 4.81 (s, 2H), 3.04 (s, 2H), 2.95-2.85 (m, 4H), 2.15-2.05 (m, 2H), 1.52 (s, 6H); ESIMS found for C₂₀H₂₂N₂O₃: m/z 339.1 (M+1).

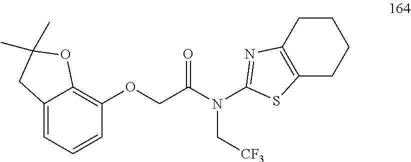 [SMILES: CC1(C)Cc2cccc(OCC(=O)N(CC(F)(F)F)c3nc4c(s3)CCCC4)c2O1]

2-((2,2-Dimethyl-2,3-dihydrobenzofuran-7-yl)oxy)-N-(4,5,6,7-tetrahydrobenzo[d]thiazol-2-yl)-N-(2,2,2-trifluoroethyl)acetamide (164) was prepared in accordance with the procedures described in Schemes 12 and 8. ¹H NMR (300 MHz, DMSO-d6): δ ppm 6.83-6.80 (m, 1H), 6.72-6.70 (m, 2H), 5.13 (s, 2H), 5.00-5.20 (m, 2H), 2.99 (s, 2H), 2.67-2.51 (m, 4H), 1.78 (broad s, 4H), 1.38 (s, 6H); ESIMS found for C₂₁H₂₃F₃N₂O₃S: m/z 441.2 (M+1).

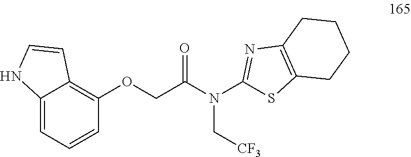 [SMILES: O=C(COc1cccc2[nH]ccc12)N(CC(F)(F)F)c1nc2c(s1)CCCC2]

2-((1H-Indol-4-yl)oxy)-N-(4,5,6,7-tetrahydrobenzo[d]thiazol-2-yl)-N-(2,2,2-trifluoroethyl)acetamide (165) was prepared in accordance with the procedures described in Schemes 12 and 1. ¹H NMR (300 MHz, DMSO-d6) δ ppm 11.12 (br, 1H), 7.26-7.24 (t, J=2.7 Hz, 1H), 7.05-7.02 (d, J 8.1 Hz, 1H), 6.98-6.93 (d, J 8.1 Hz, 1H), 6.46 (s, 1H), 6.41-6.39 (d, J 7.2 Hz, 1H), 5.23 (s, 2H), 5.15 (br, 2H), 2.66-2.59 (m, 4H), 1.77 (m, 4H); ESIMS found for C₁₉H₁₈F₃N₃O₂S: m/z 410.1 (M+1).

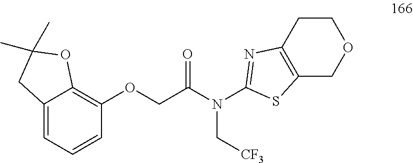 [SMILES: CC1(C)Cc2cccc(OCC(=O)N(CC(F)(F)F)c3nc4c(s3)COCC4)c2O1]

N-(6,7-Dihydro-4H-pyrano[4,3-d]thiazol-2-yl)-2-((2,2-dimethyl-2,3-dihydrobenzofuran-7-yl)oxy)-N-(2,2,2-trifluoroethyl)acetamide (166) was prepared in accordance with the procedures described in Schemes 12 and 8. ¹H NMR (300 MHz, DMSO-d6) δ ppm 6.82-6.68 (m, 3H), 5.17-5.11 (m, 4H), 4.70 (s, 2H), 3.92 (t, J=5.7 Hz, 2H), 2.98 (s, 3H), 2.72 (t, J=5.7 Hz, 2H), 1.37 (s, 6H); ESIMS found for C₂₀H₂₁F₃N₂O₄S: m/z 443.1 (M+1).

The synthesis of 5-cyclopropyl-6-methylpyridin-2-amine (LXXI) is depicted below in Scheme 31.

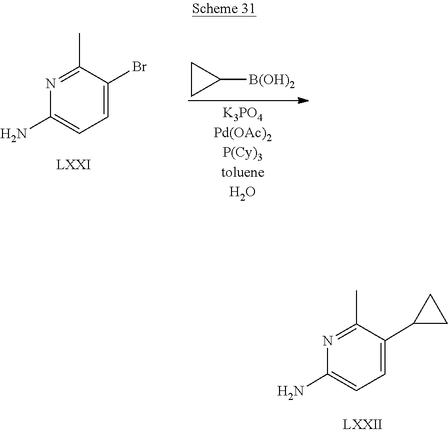

In a 250-mL 3-necked round-bottom flask purged and maintained with an inert atmosphere of nitrogen, was placed 5-bromo-6-methylpyridin-2-amine (5 g, 26.7 mmol, 1 equiv), cyclopropylboronic acid (4.6 g, 53.6 mmol, 2.00 equiv), K₃PO4 (14.2 g, 66.8 mmol, 2.5 equiv), Pd(OAc)₂ (300 mg, 1.34 mmol, 0.05 equiv), PCy₃ (750 mg, 2.67 mmol, 0.1 equiv) in 130 mL toluene and 14 mL H₂O. The resulting solution was heated to reflux for 24 hr. The resultant mixture was cooled to room temperature and concentrated under vacuum. The residue was applied onto a silica gel column with dichloromethane/methanol (10:1). This resulted in 2.9 g (73%) of 5-cyclopropyl-6-methylpyridin-2-amine as a yellow solid. ¹H NMR (300 MHz, DMSO-d6): δ ppm 7.27 (s, 1H), 7.01 (d, J=8.4 Hz), 6.21 (d, J=8.1 Hz), 5.62 (br, 2H), 2.34 (s, 3H), 1.77-1.72 (m, 1H), 0.83-0.79 (m, 2H), 0.47-0.44 (m, 1H); ESIMS found for C₉H₁₂N₂: m/z 149.2 (M+1).

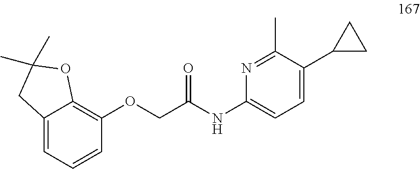 [SMILES: Cc1nc(NC(=O)COc2cccc3c2OC(C)(C)C3)ccc1C1CC1]

N-(5-Cyclopropyl-6-methylpyridin-2-yl)-2-((2,2-dimethyl-2,3-dihydrobenzofuran-7-yl)oxy)acetamide (167) was prepared from 5-cyclopropyl-6-methylpyridin-2-amine (LXXII) in accordance with the procedures described in Scheme 8. ¹H NMR (300 MHz, DMSO-d6) δ ppm 10.22 (br, 1H), 7.80 (d, J=7.8 Hz, 1H), 7.37 (d, J=8.4 Hz, 1H), 6.82-6.68 (m, 3H), 4.72 (s, 2H), 3.00 (s, 2H), 2.50 (s, 3H), 1.89 (br, 1H), 1.42 (s, 6H), 0.95-0.89 (m, 2H), 0.62-0.59 (m, 2H); ESIMS found for C₂₁H₂₄N₂O₃: m/z 353.2 (M+1).

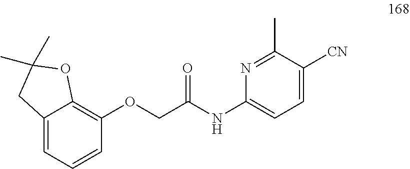 [SMILES: Cc1nc(NC(=O)COc2cccc3c2OC(C)(C)C3)ccc1C#N]

N-(5-Cyano-6-methylpyridin-2-yl)-2-((2,2-dimethyl-2,3-dihydrobenzofuran-7-yl)oxy)acetamide (168) was prepared from in accordance with the procedures described in Scheme 8. ¹H NMR (300 MHz, DMSO-d6) δ ppm 10.87 (br, 1H), 8.21 (d, J=8.7 Hz, 1H), 8.04 (d, J=8.7 Hz, 1H), 6.83-6.80 (m, 1H), 6.75-6.68 (m, 2H), 4.80 (s, 2H), 3.00 (s, 2H), 2.61 (s, 3H), 1.41 (s, 6H); ESIMS found for C₁₉H₁₉N₃O₃: m/z 338.1 (M+1).

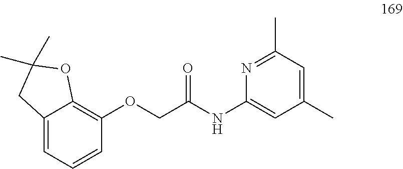

2-((2,2-Dimethyl-2,3-dihydrobenzofuran-7-yl)oxy)-N-(4,6-dimethylpyridin-2-yl)acetamide (169) was prepared in accordance with the procedures described in Scheme 8. ESIMS found for C₁₉H₂₂N₂O₃: m/z 327.1 (M+1).

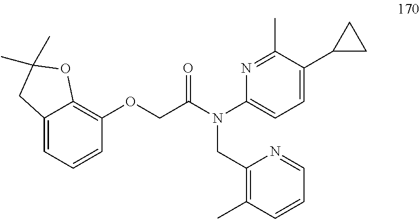 [SMILES: Cc1cccnc1CN(C(=O)COc1cccc2c1OC(C)(C)C2)c1ccc(C2CC2)c(C)n1]

N-(5-Cyclopropyl-6-methylpyridin-2-yl)-2-((2,2-dimethyl-2,3-dihydrobenzofuran-7-yl)oxy)-N-((3-methylpyridin-2-yl)methyl)acetamide (170) was prepared in accordance with the procedures described in Schemes 11 and 8. ¹H NMR (300 MHz, DMSO-d6) δ ppm 8.30 (d, J=4.8 Hz, 1H), 7.57 (d, J=7.2 Hz, 1H), 7.33 (d, J=8.1 Hz, 1H), 7.19-7.15 (m, 2H), 6.75-6.66 (m, 3H), 5.10 (s, 2H), 4.89 (s, 2H), 2.96 (s, 2H), 2.50 (s, 3H), 2.32 (s, 3H), 1.89 (br, 1H), 1.37 (s, 6H), 0.95-0.89 (m, 2H), 0.62-0.59 (m, 2H); ESIMS found for C₂₈H₃₁N₃O₃: m/z 458.2 (M+1).

Preparation of (±)-2-((2,2-dimethyl-2,3-dihydrobenzofuran-7-yl)oxy)-3-phenylpropanoic acid (LXXV) is depicted below in Scheme 31.

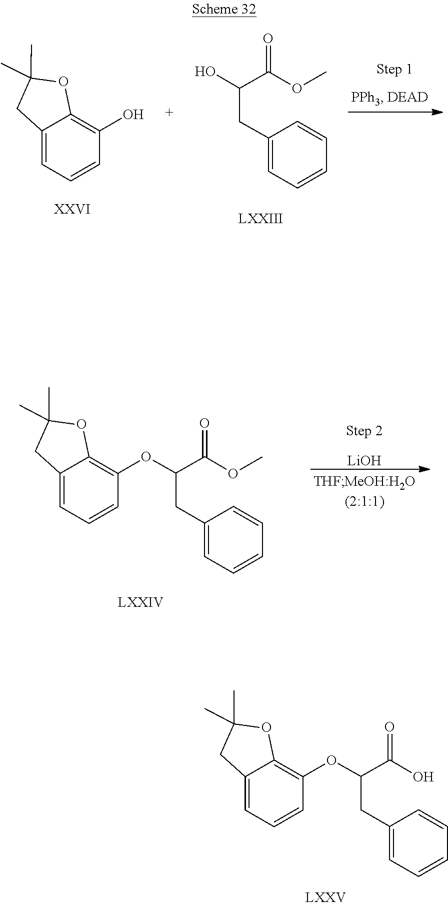

Step 1

To a solution of 2,2-dimethyl-2,3-dihydro-1-benzofuran-7-ol (XXVI) (164 mg, 1.00 mmol, 1 equiv), Ph₃P (314.4 mg, 1.20 mmol, 1.2 equiv), DCM (5 mL) was added DEAD (208.7 mg, 1.20 mmol, 1.2 equiv) dropwise with stirring at 0° C. for 30 min. To this was added (1)-methyl 2-hydroxy-3-phenylpropanoate (LXXIII) (180.0 mg, 1.00 mmol, 1.00 equiv). The resulting solution was stirred for 4 hr at room temperature. The resulting mixture was concentrated under vacuum. The residue was applied onto a silica gel column and eluted with ethyl acetate/petroleum ether (1:4). This resulted in 176 mg (54%) of (1)-methyl 2-[(2,2-dimethyl-2,3-dihydro-1-benzofuran-7-yl)oxy]-3-phenylpropanoate (LXXIV) as an off-white solid. ESIMS found for C₂₀H₂₂O₄: m/z 326.1 (M+1).

Step 2

To a mixture of (±)-methyl 2-[(2,2-dimethyl-2,3-dihydro-1-benzofuran-7-yl)oxy]-3-phenylpropanoate (LXXIV) (176 mg, 0.54 mmol, 1 equiv) and LiGH (51.7 mg, 2.16 mmol, 4.00 equiv) was added MeOH (4 mL) and H₂O (2 mL). The resulting solution was stirred for 2 hr at room temperature. The resulting mixture was concentrated under vacuum. The pH value of the solution was adjusted to 5 with HCl (1 M). The resulting mixture was extracted with 3×10 mL of ethyl acetate and the organic layers combined and dried over anhydrous sodium sulfate and concentrated under vacuum. This resulted in 144 mg (85.5%) of (±)-2-[(2,2-dimethyl-2,3-dihydro-1-benzofuran-7-yl)oxy]-3-phenylpropanoic acid (LXXV) as a white solid. ESIMS found for C₁₉H₂₀O₄: m/z 311.1 (M−1).

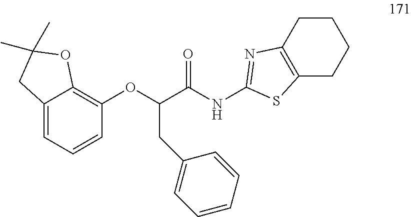 [SMILES: CC1(C)Cc2cccc(OC(Cc3ccccc3)C(=O)Nc3nc4c(s3)CCCC4)c2O1]

(±)-2-((2,2-Dimethyl-2,3-dihydrobenzofuran-7-yl)oxy)-3-phenyl-N-(4,5,6,7-tetrahydrobenzo[d]thiazol-2-yl)propanamide (171) was prepared in accordance with the procedures described in Scheme 8. ¹H NMR (300 MHz, DMSO-d6) δ ppm 9.9-9.6 (broad s, 1H), 7.39-7.18 (m, 5H), 6.79-6.76 (m, 1H), 6.74-6.60 (m, 2H), 5.08 (t, J=6.6 Hz, 1H), 3.17 (d, J=6.6 Hz, 2H), 2.95 (s, 2H), 2.62 (br, 2H), 2.54 (br, 2H), 1.76 (br, 4H), 1.41 (s, 3H), 1.35 (s, 3H); ESIMS found for C₂₆H₂₈N₂O₃S: m/z 449.1 (M+1).

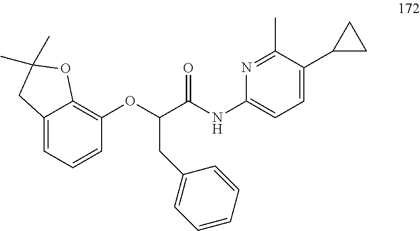 [SMILES: Cc1nc(NC(=O)C(Cc2ccccc2)Oc2cccc3c2OC(C)(C)C3)ccc1C1CC1]

(±)-N-(5-Cyclopropyl-6-methylpyridin-2-yl)-2-((2,2-dimethyl-2,3-dihydrobenzofuran-7-yl)oxy)-3-phenylpropanamide (172) was prepared in accordance with the procedures described in Scheme 8. ¹H NMR (300 MHz, DMSO-d6) δ ppm 10.29 (br, 1H), 7.81 (d, J=8.4 Hz, 1H), 7.38-7.17 (m, 6H), 6.78-6.75 (m, 1H), 6.66-6.61 (m, 2H), 5.09-5.07 (m, 1H), 3.18-3.13 (m, 2H), 2.96 (s, 2H), 2.51 (s, 3H), 1.90-1.85 (m, 1H), 1.40 (s, 3H), 1.38 (s, 3H), 0.94-0.88 (m, 2H), 0.61-0.56 (m, 2H); ESIMS found for C₂₈H₃₀N₂O₃: m/z 443.2 (M+1).

Preparation of 2-((2,2-dimethyl-5-(trifluoromethyl)-2,3-dihydrobenzofuran-7-yl)oxy)acetic acid (LXXXII) is depicted below in Scheme 32.

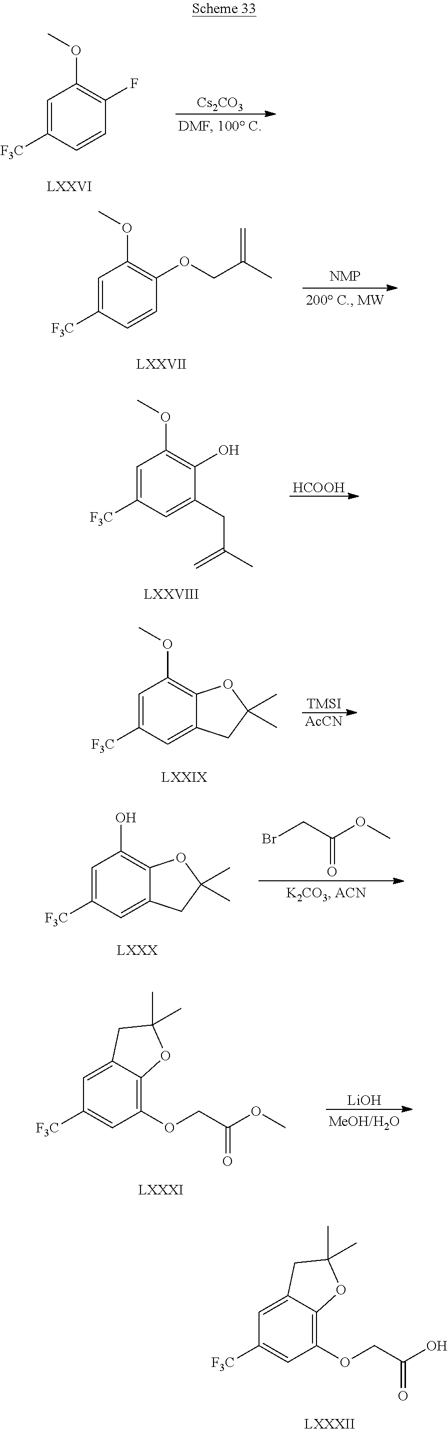

Step 1

A mixture of 1-fluoro-2-methoxy-4-(trifluoromethyl)benzene (LXXVI) (1.94 g, 9.99 mmol, 1 equiv), 2-methylprop-2-en-1-ol (3.6 g, 0.05 mmol, 5 equiv), Cs₂CO₃ (6.5 g, 19.95 mmol, 2.00 equiv), DMF (30 mL). The resulting solution was stirred for 12 hr at 100° C. in an oil bath. The resulting mixture was diluted with 50 mL of water and extracted with 2×30 mL of ethyl acetate and the organic layers combined and dried over anhydrous sodium sulfate and concentrated. The residue was applied onto a silica gel column and eluted with ethyl acetate/petroleum ether (1:5). This resulted in 1.25 g (50.80%) of 2-methoxy-1-((2-methylprop-2-en-1-yl)oxy)-4-(trifluoromethyl)benzene (LXXVII) as colorless oil. ¹H NMR (300 MHz, CHLOROFORM-d) δ ppm 7.22-7.20 (m, 1H), 7.18 (s, 1H), 7.11-6.92 (m, 1H), 5.12 (d, J=3.0 Hz, 1H), 5.04 (d, J=3.0 Hz, 1H), 4.59 (s, 2H), 3.96 (s, 3H), 1.85 (s, 3H).

Step 2

Into a 30-mL sealed tube, was placed 2-methoxy-1-((2-methylprop-2-en-1-yl)oxy)-4-(trifluoromethyl)benzene (LXXVII)(1.25 g, 1.0 equiv), NMP (10 mL). The resulting solution was stirred for 2 hr at 200° C. heating with microwave. The resulting mixture was diluted with 20 ml of water and extracted with 2×10 mL of ethyl acetate and the organic layers combined and dried over anhydrous sodium sulfate and concentrated under vacuum. The residue was applied onto a silica gel column and eluted with ethyl acetate/petroleum ether (1:10). This resulted in 790 mg of 2-methoxy-6-(2-methylprop-2-en-1-yl)-4-(trifluoromethyl)phenol (LXXVIII) as yellow oil. ¹H NMR (300 MHz, CHLOROFORM-d) δ ppm 7.07 (s, 1H), 6.98 (s, 1H), 5.98 (s, 1H), 4.87 (d, J=3.0 Hz, 1H), 4.72 (d, J=3.0 Hz, 1H), 3.97 (s, 3H), 3.41 (s, 2H), 1.76 (s, 3H). ESIMS found for C₁₂H₁₃F₃O₂: m/z 247.2 (M+1).

Step 3

A solution of 2-methoxy-6-(2-methylprop-2-en-1-yl)-4-(trifluoromethyl)phenol (LXXVIII) (790 mg, 1 equiv), formic acid (10 mL). The resulting solution was stirred for 24 hr at room temperature. The resulting mixture was concentrated under vacuum. The residue was washed diluted with 10 ml of water and extracted with 2×10 mL of EA, The organic layer was combined, dried over anhydrous sodium sulfate and concentrated under the vacuum. The residue was applied onto a silica gel column eluting with ethyl acetate/petroleum ether (1:5). This resulted in 642 mg of 7-methoxy-2,2-dimethyl-5-(trifluoromethyl)-2,3-dihydro-1-benzofuran (LXXIX) as a white solid. ¹H NMR (300 MHz, DMSO-d6) δ ppm 7.17 (broad s, 1H), 7.09 (broad s, 1H), 3.82 (s, 3H), 3.08 (s, 2H), 1.44 (s, 6H); ESIMS found for C₁₂H₁₃F₃O₂: m/z 247.2 (M+1).

Step 4

A solution of 7-methoxy-2,2-dimethyl-5-(trifluoromethyl)-2,3-dihydro-1-benzofuran (LXXIX) (500 mg, 2.03 mmol, 1.0 equiv) in AcCN (5 mL) was heated to 70° C. in an oil bath. This was followed by the addition of freshly prepared TMSI (2 N) (5 mL, 5.00 equiv) in portions. The resulting solution was stirred for 2 hr and was monitored by LC-MS, then additional TMSI (4 mL, 2N, 4.0 equiv) was added twice until the starting material was completely consumed. The resulting mixture was concentrated under vacuum. The residue was applied onto a silica gel column and eluted with ethyl acetate/petroleum ether (1:5). This resulted in 278 mg (59%) of 2,2-dimethyl-5-(trifluoromethyl)-2,3-dihydro-1-benzofuran-7-ol (LXXX) as a white solid. ESIMS found for C₁₁H₁₃F₃O₂: m/z 231.2 (M−1).

Step 5

A mixture of 2,2-dimethyl-5-(trifluoromethyl)-2,3-dihydro-1-benzofuran-7-ol (LXXX) (84 mg, 0.36 mmol, 1 equiv), K₂CO₃ (100 mg, 0.72 mmol, 2 equiv), methyl 2-bromoacetate (110.7 mg, 0.72 mmol, 2.00 equiv) in 4 mL AcCN was stirred for 4 hr at 60° C., and then concentrated under vacuum. The residue was applied onto a silica gel column and eluted with ethyl acetate/petroleum ether (1:4). This resulted in 110 mg (99.9%) of methyl 2-((2,2-dimethyl-5-(trifluoromethyl)-2,3-dihydro-1-benzofuran-7-yl)oxy)acetate (LXXXI) as a white solid. ¹H NMR (300 MHz, CHLOROFORM-d) δ ppm 7.11 (s, 1H), 6.99 (s, 1H), 4.78 (s, 2H), 3.82 (s, 3H), 3.07 (s, 2H), 1.55 (s, 6H); ESIMS found for C₁₄H₁₅F₃O₄: m/z 305.2 (M+1).

Step 6

A mixture of methyl 2-((2,2-dimethyl-5-(trifluoromethyl)-2,3-dihydro-1-benzofuran-7-yl)oxy)acetate (LXXXI) (110 mg, 0.36 mmol, 1 equiv), LiGH (34.6 mg, 1.44 mmol, 4.00 equiv), MeOH (2 mL), THE (2 mL), H₂O (1 mL). The resulting solution was stirred for 4 hr at room temperature. The resulting mixture was concentrated under vacuum. The pH value of the solution was adjusted to 5 with HCl (1 mol/L). The solids were collected by filtration. The solid was dried in an oven under reduced pressure. This resulted in 101 mg (96.26%) of 2-((2,2-dimethyl-5-(trifluoromethyl)-2,3-dihydro-1-benzofuran-7-yl)oxy)acetic acid (LXXXII) as a white solid. ESIMS found for C₁₃H₁₃F₃O₄: m/z 289.2 (M−1).

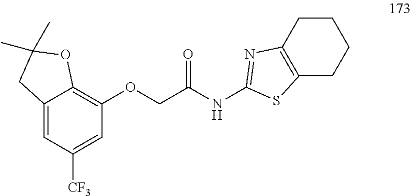 [SMILES: CC1(C)Cc2cc(C(F)(F)F)cc(OCC(=O)Nc3nc4c(s3)CCCC4)c2O1]

2-((2,2-Dimethyl-5-(trifluoromethyl)-2,3-dihydrobenzofuran-7-yl)oxy)-N-(4,5,6,7-tetrahydrobenzo[d]thiazol-2-yl)acetamide (173) was prepared in accordance with the procedures described in Scheme 8. ¹H NMR (300 MHz, DMSO-d6) δ ppm 12.07 (br, 1H), 7.21 (s, 1H), 7.09 (s, 1H), 4.89 (s, 2H), 3.08 (s, 2H), 2.63 (br, 2H), 2.56 (br, 2H), 1.77 (br, 4H), 1.44 (s, 6H); ESIMS found for C₂₀H₂₁F₃N₂O₃S: m/z 427.1 (M+1).

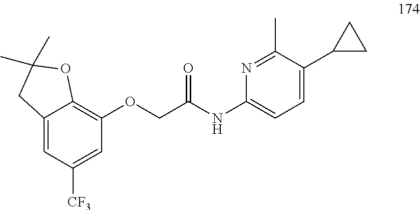 [SMILES: Cc1nc(NC(=O)COc2cc(C(F)(F)F)cc3c2OC(C)(C)C3)ccc1C1CC1]

N-(5-Cyclopropyl-6-methylpyridin-2-yl)-2-((2,2-dimethyl-5-(trifluoromethyl)-2,3-dihydrobenzofuran-7-yl)oxy)acetamide (174) was prepared in accordance with the procedures described in Scheme 8. ¹H NMR (300 MHz, DMSO-d6) δ ppm 10.37 (br, 1H), 7.78 (d, J=8.4 Hz, 1H), 7.36 (d, J=8.4 Hz, 1H), 7.20 (s, 1H), 7.10 (s, 1H), 4.83 (s, 2H), 3.08 (s, 2H), 2.51 (s, 3H), 1.91-1.84 (m, 1H), 1.45 (s, 6H), 0.94-0.88 (m, 2H), 0.66-0.61 (m, 2H); ESIMS found for C₂₂H₂₃F₃N₂O₃: m/z 421.1 (M+1).

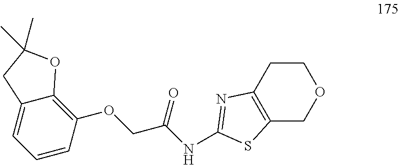 [SMILES: CC1(C)Cc2cccc(OCC(=O)Nc3nc4c(s3)COCC4)c2O1]

N-(6,7-Dihydro-4H-pyrano[4,3-d]thiazol-2-yl)-2-((2,2-dimethyl-2,3-dihydrobenzofuran-7-yl)oxy)acetamide (175) was prepared in accordance with the procedures described in Scheme 8. 22% yield. ¹H NMR (400 MHz, CHLOROFORM-d) δ ppm 1.52 (s, 6H), 2.77-2.83 (m, 2H), 3.04 (s, 2H), 4.03 (t, J=5.56 Hz, 2H), 4.77 (s, 2H), 4.78-4.80 (m, 2H), 6.77 (d, J=2.45 Hz, 1H), 6.78 (s, 1H), 6.86-6.91 (m, 1H), 9.96 (br s, 1H); ESIMS found for C₁₈H₂₀N₂O₄S: m/z 361.0 (M+1).

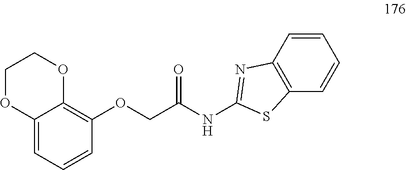 [SMILES: O=C(COc1cccc2c1OCCO2)Nc1nc2ccccc2s1]

N-(Benzo[d]thiazol-2-yl)-2-((2,3-dihydrobenzo[b][1,4]dioxin-5-yl)oxy) acetamide (176) was prepared in accordance with the procedures described in Scheme 8. ¹H NMR (400 MHz, CHLOROFORM-d) δ ppm 4.28-4.36 (m, 2H), 4.43-4.49 (m, 2H), 4.79 (s, 2H), 6.60 (d, J=8.19 Hz, 1H), 6.67 (d, J=8.31 Hz, 1H), 6.77-6.85 (m, 1H), 7.30-7.37 (m, 1H), 7.46 (t, J=7.70 Hz, 1H), 7.78-7.88 (m, 2H), 10.45 (br s, 1H); ESIMS found for C₁₇H₁₄N₂O₄S: m/z 343.2 (M+1).

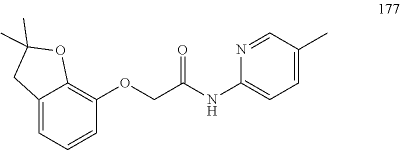

2-((2,2-Dimethyl-2,3-dihydrobenzofuran-7-yl)oxy)-N-(5-methylpyridin-2-yl) acetamide (177) was prepared in accordance with the procedures described in Scheme 8. 16% yield. ¹H NMR (400 MHz, CHLOROFORM-d) δ ppm 1.51 (s, 6H), 2.30 (s, 3H), 3.03 (s, 2H), 4.70 (s, 2H), 6.73-6.78 (m, 1H), 6.79-6.82 (m, 1H), 6.86 (dd, J=7.09, 0.98 Hz, 1H), 7.53 (dd, J=8.44, 2.32 Hz, 1H), 8.12 (d, J=2.20 Hz, 1H), 8.15 (d, J=8.44 Hz, 1H), 9.02 (br s, 1H); ESIMS found for C₁₈H₂₀N₂O₃: m/z 313.3 (M+1).

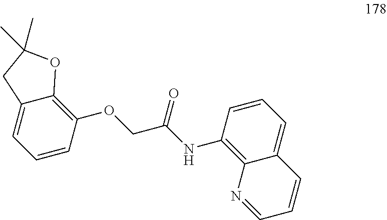 [SMILES: CC1(C)Cc2cccc(OCC(=O)Nc3cccc4cccnc34)c2O1]

2-((2,2-Dimethyl-2,3-dihydrobenzofuran-7-yl)oxy)-N-(quinolin-8-yl) acetamide (178) was prepared in accordance with the procedures described in Scheme 8 in 12% yield. ESIMS found for C₂₁H₂₀N₂O₃: m/z 349.0 (M+1).

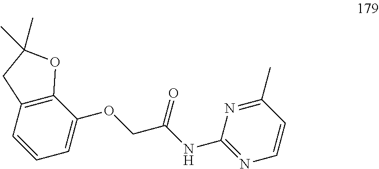 [SMILES: Cc1ccnc(NC(=O)COc2cccc3c2OC(C)(C)C3)n1]

2-((2,2-Dimethyl-2,3-dihydrobenzofuran-7-yl)oxy)-N-(4-methylpyrimidin-2-yl)acetamide (179) was prepared in accordance with the procedures described in Scheme 9. 28% yield. ¹H NMR (400 MHz, CHLOROFORM-d) δ ppm 1.51 (s, 6H), 2.50 (s, 3H), 3.04 (s, 2H), 4.78 (s, 2H), 6.74-6.79 (m, 1H), 6.80-6.85 (m, 1H), 6.87 (dd, J=7.21, 0.86 Hz, 1H), 6.92 (d, J=5.01 Hz, 1H), 8.53 (d, J=5.01 Hz, 1H), 9.19 (br s, 1H); ESIMS found for C₁₇H₁₉N₃O₃: m/z 314.3 (M+1).

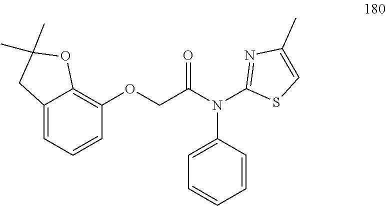 [SMILES: Cc1csc(N(C(=O)COc2cccc3c2OC(C)(C)C3)c2ccccc2)n1]

2-((2,2-Dimethyl-2,3-dihydrobenzofuran-7-yl)oxy)-N-(4-methylthiazol-2-yl)-N-phenylacetamide (180) was prepared in accordance with the procedures described in Schemes 12 and 9. ¹H NMR (400 MHz, CHLOROFORM-d) δ ppm 1.44 (s, 6H), 2.23 (s, 3H), 2.98 (s, 2H), 4.63 (s, 2H), 6.59 (s, 1H), 6.69-6.73 (m, 2H), 6.76-6.81 (m, 1H), 7.34-7.40 (m, 2H), 7.50-7.60 (m, 3H); ESIMS found for C₂₂H₂₂N₂O₃S: m/z 395.3 (M+1).

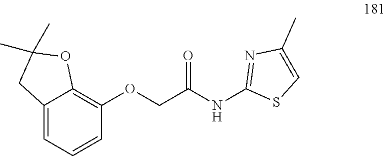 [SMILES: Cc1csc(NC(=O)COc2cccc3c2OC(C)(C)C3)n1]

2-((2,2-Dimethyl-2,3-dihydrobenzofuran-7-yl)oxy)-N-(4-methylthiazol-2-yl) acetamide (181) was prepared in accordance with the procedures described in Scheme 27. ¹H-NMR (500 MHz, CDCl₃): δ 1.52 (s, 6H), 2.41 (s, 3H), 3.04 (s, 2H), 4.76 (s, 2H), 6.77 (m, 2H), 6.88 (m, 1H), 7.11 (d, J=1.26 Hz, 1H).

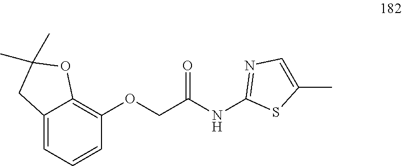

2-((2,2-Dimethyl-2,3-dihydrobenzofuran-7-yl)oxy)-N-(5-methylthiazol-2-yl) acetamide (182) was prepared in accordance with the procedures described in Scheme 27. ¹H NMR (500 MHz, CDCl₃): δ 1.52 (s, 6H), 2.36 (s, 3H), 3.04 (s, 2H), 4.77 (s, 2H), 6.56 (d, J=0.95 Hz, 1H), 6.77 (m, 2H), 6.88 (m, 1H).

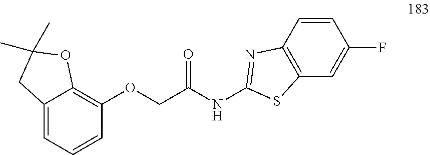 [SMILES: CC1(C)Cc2cccc(OCC(=O)Nc3nc4ccc(F)cc4s3)c2O1]

2-((2,2-Dimethyl-2,3-dihydrobenzofuran-7-yl)oxy)-N-(6-fluorobenzo[d]thiazol-2-yl)acetamide (183) was prepared in accordance with the procedures described in Scheme 27. ¹H NMR (500 MHz, CDCl₃): δ 1.54 (s, 6H), 3.06 (s, 2H), 4.82 (s, 2H), 6.81 (m, J=5.04, 2H), 6.91 (m, 1H), 7.51 (m, 1H), 7.74 (m, 1H), 10.23 (s, 1H).

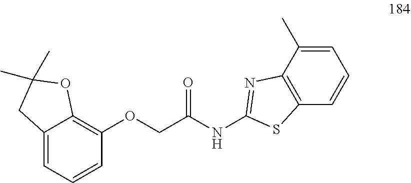 [SMILES: Cc1cccc2sc(NC(=O)COc3cccc4c3OC(C)(C)C4)nc12]

2-((2,2-Dimethyl-2,3-dihydrobenzofuran-7-yl)oxy)-N-(4-methylbenzo[d]thiazol-2-yl)acetamide (184) was prepared in accordance with the procedures described in Scheme 27. ¹H NMR (500 MHz, CDCl₃): δ 1.57 (s, 6H), 2.66 (s, 3H), 3.07 (s, 2H), 4.83 (s, 2H), 6.86 (m, 3H), 7.24 (m, 2H), 7.67 (d, J=7.25 Hz, 1H).

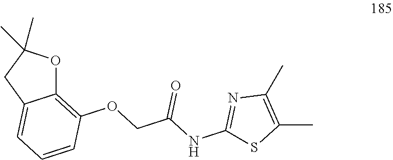

2-((2,2-Dimethyl-2,3-dihydrobenzofuran-7-yl)oxy)-N-(4,5-dimethylthiazol-2-yl)acetamide (185) was prepared in accordance with the procedures described in Scheme 27. ¹H NMR (500 MHz, CDCl₃): δ 1.44 (s, 6H), 2.16 (s, 3H), 2.22 (s, 3H), 2.96 (s, 2H), 4.68 (s, 2H), 6.69 (m, 2H), 6.79 (m, 1H).

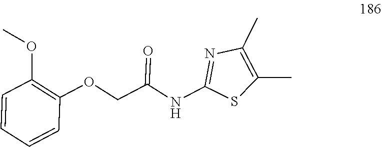 [SMILES: COc1ccccc1OCC(=O)Nc1nc(C)c(C)s1]

N-(4,5-Dimethylthiazol-2-yl)-2-(2-methoxyphenoxy)acetamide (186) was prepared in accordance with the procedures described in Scheme 27. ¹H NMR (500 MHz, CDCl₃): δ 2.17 (s, 3H), 2.23 (s, 3H), 3.87 (s, 3H), 4.64 (s, 2H), 6.87 (m, 3H), 6.99 (m, 1H), 10.72 (s, 1H).

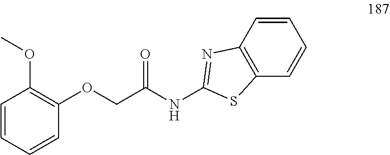 [SMILES: COc1ccccc1OCC(=O)Nc1nc2ccccc2s1]

N-(Benzo[d]thiazol-2-yl)-2-(2-methoxyphenoxy)acetamide (187) was prepared in accordance with the procedures described in Scheme 27. ¹H-NMR (500 MHz, CDCl₃): δ 3.13 (s, 3H), 4.79 (s, 2H), 7.03 (dd, J=8, 1.4 Hz, 1H), 7.1 (m, 1H), 7.33 (m, 1H), 7.46 (m, 1H), 7.83 (m, 2H).

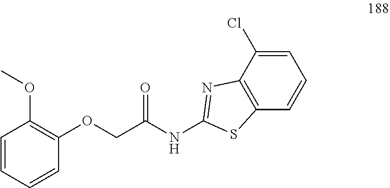 [SMILES: COc1ccccc1OCC(=O)Nc1nc2c(Cl)cccc2s1]

N-(4-Chlorobenzo[d]thiazol-2-yl)-2-(2-methoxyphenoxy)acetamide (188) was prepared in accordance with the procedures described in Scheme 27. ¹H NMR (500 MHz, CDCl₃): δ 4.05 (s, 3H), 4.80 (s, 2H), 6.96 (m, 2H), 7.04 (d, J=7.88 Hz, 1H), 7.10 (m, 1H), 7.25 (m, 1H), 7.48 (d, J=7.88 Hz, 1H), 7.73 (d, J=7.88 Hz, 1H).

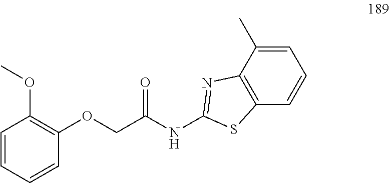 [SMILES: COc1ccccc1OCC(=O)Nc1nc2c(C)cccc2s1]

2-(2-Methoxyphenoxy)-N-(4-methylbenzo[d]thiazol-2-yl)acetamide (189) was prepared in accordance with the procedures described in Scheme 27. ¹H-NMR (500 MHz, CDCl₃): δ 2.67 (s, 3H), 4.06 (s, 3H), 4.80 (s, 2H), 5.30 (s, 2H), 6.9 (m, 2H), 7.06 (m, 2H), 7.11 (m, 1H), 7.23 (m, 2H), 7.67 (d, 1H), 10.61 (s, 1H).

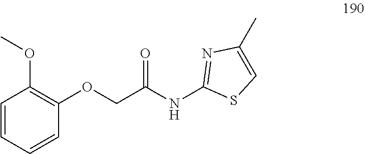 [SMILES: COc1ccccc1OCC(=O)Nc1nc(C)cs1]

2-(2-Methoxyphenoxy)-N-(4-methylthiazol-2-yl)acetamide (190) was prepared in accordance with the procedures described in Scheme 27. ¹H-NMR (500 MHz, CDCl₃): δ 2.36 (d, 3H), 3.96 (s, 3H), 4.74 (s, 2H), 6.55 (d, J=0.95 Hz, 1H), 6.95 (m, 3H), 7.07 (m, 1H), 10.46 (s, 1H).

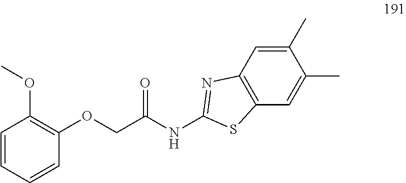 [SMILES: COc1ccccc1OCC(=O)Nc1nc2cc(C)c(C)cc2s1]

N-(5,6-Dimethylbenzo[d]thiazol-2-yl)-2-(2-methoxyphenoxy)acetamide (191) was prepared in accordance with the procedures described in Scheme 27. ¹H NMR (500 MHz, CDCl₃): δ 2.3 (s, 6H), 3.93 (s, 3H), 4.70 (s, 2H), 6.80 (m, 2H), 6.90 (dd, 1H), 7.02 (m, 1H), 7.52 (s, 2H), 10.52 (s, 1H).

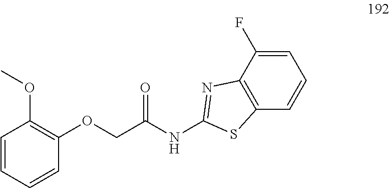 [SMILES: COc1ccccc1OCC(=O)Nc1nc2c(F)cccc2s1]

N-(4-Fluorobenzo[d]thiazol-2-yl)-2-(2-methoxyphenoxy)acetamide (192) was prepared in accordance with the procedures described in Scheme 27. ¹H NMR (500 MHz, CDCl₃): δ 3.94 (s, 3H), 4.73 (s, 2H), 6.89 (m, 2H), 6.95 (dd, J=8.04, 1.42 Hz, 1H), 7.03 (m, 1H), 7.09 (dd, J=9.93, 7.72 Hz, 1H), 7.10 (dd, J=11.3, 8.51 Hz, 1H), 7.21 (m, 1H), 7.53 (dd, J=7.88, 0.95, 1H), 10.72 (s, 1H).

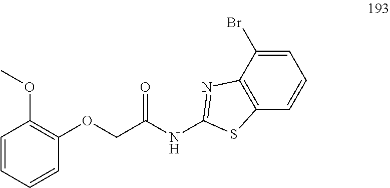 [SMILES: COc1ccccc1OCC(=O)Nc1nc2c(Br)cccc2s1]

N-(4-Bromobenzo[d]thiazol-2-yl)-2-(2-methoxyphenoxy)acetamide (193) was prepared in accordance with the procedures described in Scheme 27. ¹H NMR (500 MHz, CDCl₃): δ 4.01 (s, 3H), 4.74 (s, 2H), 6.91 (m, 2H), 6.98 (m, 1H), 7.04 (m, 1H), 7.12 (t, J=7.88 Hz, 1H), 7.58 (dd, J=7.57, 0.95 Hz, 1H), 7.70 (dd, J=7.88, 0.95 Hz, 1H), 11.01 (s, 1H).

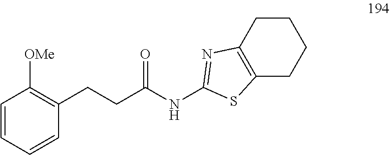 [SMILES: COc1ccccc1CCC(=O)Nc1nc2c(s1)CCCC2]

3-(2-Methoxyphenyl)-N-(4,5,6,7-tetrahydrobenzo[d]thiazol-2-yl) propanamide (194) was prepared in accordance with the procedures described in Scheme 8 from 3-(2-methoxyphenyl)propanoic acid. ESIMS found for C₁₇H₂₀N₂O₂S: m/z 317.2 (M+H).

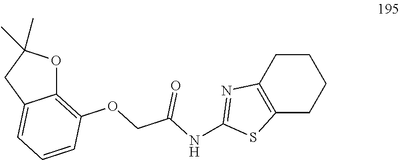 [SMILES: CC1(C)Cc2cccc(OCC(=O)Nc3nc4c(s3)CCCC4)c2O1]

2-((2,2-Dimethyl-2,3-dihydrobenzofuran-7-yl)oxy)-N-(4,5,6,7-tetrahydrobenzo[d]thiazol-2-yl)acetamide (195) was prepared in accordance with the procedures described in Scheme 27. ¹H NMR (500 MHz, CDCl₃): δ 1.42 (s, 6H), 1.76 (m, 4H), 2.54 (s, 2H), 2.62 (s, 2H), 2.96 (s, 2H), 4.64 (s, 2H), 6.66-6.74 (m, 2H), 6.81 (dd, J=7.09, 1.10 Hz, 1H).

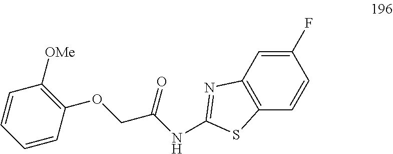 [SMILES: COc1ccccc1OCC(=O)Nc1nc2cc(F)ccc2s1]

N-(5-Fluorobenzo[d]thiazol-2-yl)-2-(2-methoxyphenoxy)acetamide (196) was prepared in accordance with the procedures described in Scheme 27. ¹H NMR (500 MHz, CDCl₃): δ 3.95 (s, 3H), 4.72 (s, 2H), 6.90 (m, 2H), 6.97 (m, 1H), 7.02 (m, 1H), 7.43 (dd, J=9.46, 2.21 Hz, 1H), 7.67 (m, 1H), 10.72 (s, 1H).

### Example 2

The above synthesized compounds were screened for their ability to inhibit the downmodulation of MHC-I molecules expressed on the surface of HIV infected SupT1 cells. HIV-1 infection was initiated using the assay procedure described below.

Two reporter viruses were used for these assays. A wild-type HIV-1 (NL4.3) virus carrying GFP (NL4.3-GFP), was used for easy identification of infected cells by monitoring GFP levels using flow cytometry. In this NL4.3-GFP reporter virus, GFP has been inserted in the nef coding region, whereas the nef gene is expressed from an internal ribosomal entry site (IRES) derived from the encephalomyocarditis virus (EMCV). [Arganaraz, E. R., Cortes M. J., Leibel, S. and Lama, J. 2002. Human Immunodeficiency type 1 Vpr protein does not modulate surface expression of the CD4 receptor. J. Virol. 76(8):4125-30]. A second HIV-1 reporter virus is a variant of NL4.3-GFP, in which a deletion eliminates the nef gene and therefore it does not express the viral protein Nef involved in downmodulation of MHC-I. This HIV-1 virus is named NL4.3-ΔNef-GFP (“ΔNef”), and it is used as a control for absence of downmodulation of MHC-I. Both viruses were generated by transfection of 293T cells with proviral plasmids and then infectious particles were harvested from the tissue culture media after 48 hours. Nef-defective HIV-1 viruses are significantly less infectious than the wild-type counterparts. To increase the infection rate of the ΔNef variant, this reporter virus was pseudotyped during production in 293T cells with the envelope glycoprotein of the vesicular stomatitis virus (VSV).

SupT1 cells were distributed at 200,000 cells per well into 96 well multiwell plates. Cells were then infected with the NL4.3-GFP or ΔNef virus variant using a spinoculation method [Cortés, M. J.; Wong-Staal, F.; Lama, J. 2002. “Cell surface CD4 interferes with the infectivity of HIV-1 particles released from T cells.” J. Biol. Chem., 277(3), 1770-9]. This procedure relies on centrifugation of the mixture of cells and viruses at 37° C. for 90 minutes to increase the infection rate of HIV-1. Cells and virus are incubated in RPMI media supplemented with 10% fetal bovine serum (FBS) in the presence of 4 μg per ml of polybrene. After centrifugation, the cells are incubated at 37° C. in 5% CO₂ for 30 minutes to allow the cells to recover and let the virus complete entry into cells. The culture media in each well was then replaced with fresh media and cells were incubated for 20 h before addition of the test compounds at specific concentrations. Compounds were added to the wells as a 4-fold or 5-fold serial dilution starting from 25 μM and were maintained for 24 more hours until cells were stained with a specific antibody to detect MHC-I (recognizing HLA-A2). In some instances, compounds were added to the wells as a 3-fold serial dilution starting from 30 uM. A series of control wells received only culture media with vehicle (0.1% DMSO). To detect HLA-A2 cells were stained for 30 min with a biotinylated HLA-A2/28 antibody (One Lambda, clone BIH0037), washed and then incubated with streptavidin conjugated with APC (Streptavidin APC, EBiosciences, 17-4317) to reveal the anti-HLA-A2 bound antibody. After washing the cells again they were fixed with 0.5% paraformaldehyde and submitted to flow cytometry to determine the extent of HIV-1 infection (GFP fluorescence signal) and MHC-I surface expression (APC fluorescence).

MHC type I levels on the cell surface and the levels of GFP in the cells were measured by flow cytometry using gates set with pre-determined criteria to separate cell populations based on their mean fluorescence intensity (MFI) levels for MHC-I and GFP.

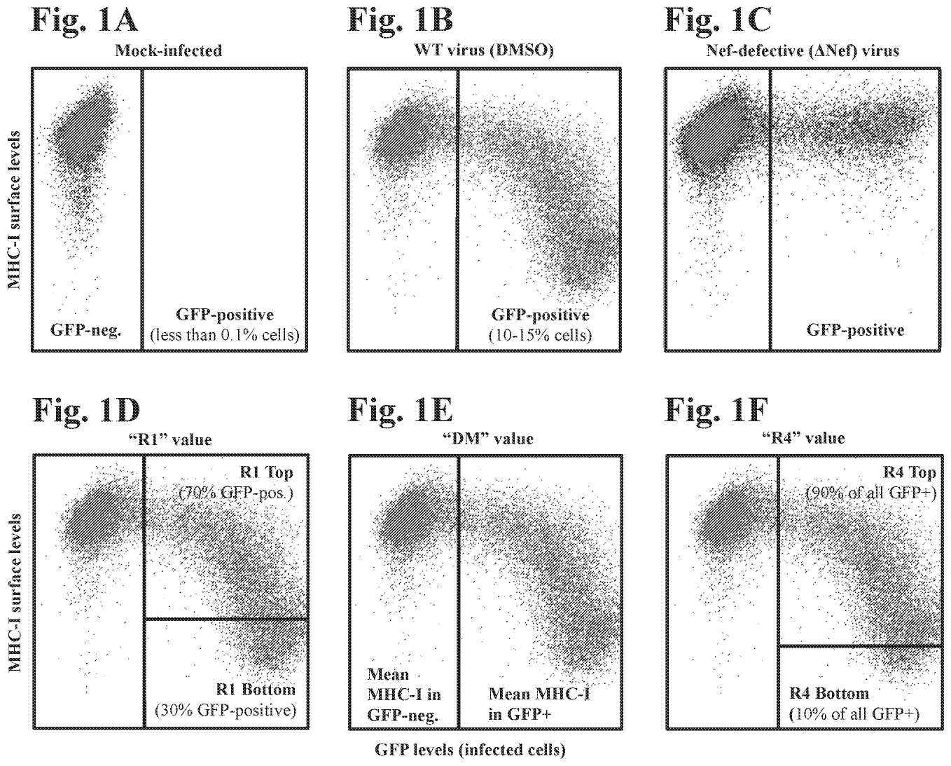

The type of gates and criteria to separate cells with different levels of expression of MHC-I and GFP are represented in FIG. 1. Flow cytometry data was analyzed only on live cells, which were estimated by comparing the forward scatter (FS) and side scatter (SS) parameters obtained with the flow cytometry device. Uninfected (“mock-infected”) controls (FIG. 1A) were used to set the background levels of expression of GFP found in these cells. The criteria for GFP positivity was set to include no more than 0.1% of the uninfected cells, whereas under typical experimental conditions 10-15% of the cells incubated with wild-type virus (NL4.3-GFP) were positive for GFP-expression. (FIG. 1B). Cells incubated with the ΔNef variant also expressed GFP, usually to larger levels due to the greater extent of infection promoted by pseudotyping with the VSV glycoprotein (FIG. 1C). However, unlike cells infected with wild-type virus, cells incubated with the ΔNef virus variant failed to downmodulate MHC-I, which remain unchanged even in infected cells expressing high levels of GFP.

To determine the extent of downmodulation of MHC-I induced by HIV-1 infection three different parameters were used: “R1”, “R4” and “DM”. These values were reduced when downmodulation of MHC-I was inhibited by test compounds, or in infections with the ΔNef virus. The criteria to select gates and the formulas used to generate these values were as follows.

To determine “R1”, an “R1 Top” region in flow cytometry dot plots was set to include 70% of all GFP-positive cells. The “R1 Bottom” region contained the remaining 30% of the GFP-positive cells. Cells in the R1 bottom area expressed lower levels of surface MHC-I (FIG. 1D). The same areas (R1-Top and R1-Bottom), were used in flow cytometry dot plots of infections with ΔNef virus to calculate the background levels of MHC-I downmodulation. The R1 value in a given sample was calculated by dividing the number of cells in the “R1-Bottom” area by the number of cells in the R1 Top area, and multiplying by 100. Then, the averaged R1 background value of MHC-I downmodulation obtained in infections with NL4.3ΔNef-GFP virus was subtracted to the number calculated in the samples infected with wild type (NL4.3-GFP). “R1” values obtained in the presence or absence of test compounds were calculated and expressed as a percentage value of those observed in samples incubated with vehicle alone (0.1% DMSO).

To determine “R4”, an “R4 Top” region in flow cytometry dot plots was set to include 90% of all GFP-positive cells. The “R4 Bottom” region contained the remaining 10% of the GFP-positive cells, which expressed levels of MHC-I approximately 50 to 100-fold time lower than those observed in uninfected cells (FIG. 1F). The same “R4-Top” and “R4-Bottom” areas were also set in infections with ΔNef virus to calculate the background levels of MHC-I downmodulation. The R4 value in a given sample was then calculated by dividing the number of cells in the “R4-Bottom” area by the number of cells in the “R4-Top” area and multiplying by 100. Then, the averaged background value of MHC-I downmodulation generated from infections with NL4.3ΔNef-GFP virus was subtracted to the number calculated in the samples infected with wild type (NL4.3-GFP). “R4” values were then generated in the presence or absence of test compounds. The “R4” value for a given data-point is expressed as a percentage value of those observed in samples incubated with vehicle alone (0.1% DMSO).

Another value to determine the extent of downmodulation (“DM value”) was obtained by comparing the MFI levels of surface MHC-I in infected cells (GFP-positive cells) with those found in uninfected (GFP-negative) in the same sample well. To determine the “DM” value the mean level of surface MHC-I in all uninfected cells was divided by the mean levels of surface MHC-I in all infected cells (FIG. 1E). The background “DM” value observed in samples infected with nef-defective virus (NL4.3ΔNef-GFP) was subtracted from all samples. The “DM” value for a given data-point was expressed as the percentage value of those observed in samples incubated with vehicle alone (0.1% DMSO) in the absence of test compound.

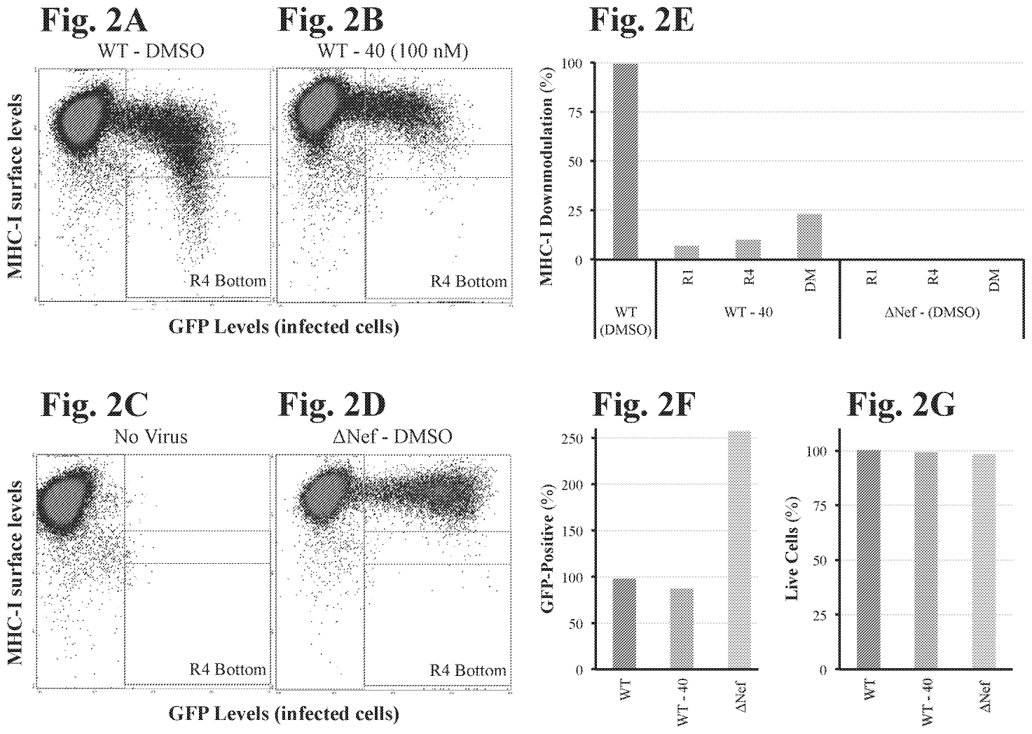

FIG. 2 shows the effect of a representative inhibitor (compound #40) in the level of downmodulation of MHC-I induced by HIV infection in SupT1 cells. FIG. 2A shows the level of downmodulation in cells infected with wild-type virus in the presence of vehicle alone. A significant number of cells with reduced levels of surface MHC-I expression was found in the “R4 Bottom” region. In contrast, incubation of infected cells with 100 nM compound #40 results in a reduction in the number of cells with low levels of MHC-I (“R4-Bottom” area), which translated in R4 values approximately 10-fold lower than in cells treated with DMSO only (FIGS. 2B and 2E). Similar reductions were also observed when the “R1” or “DM” values were used to monitor MHC-I downmodulation activity. R1 and DM values in compound-treated cells infected with wild-type virus were similar to the background levels of downmodulation observed in cells infected with a nef-defective virus (FIG. 2E). Importantly, the effect of compound #40 occurred under conditions in which neither the level of HIV-1 infection (percentage of GFP-positive cells, as shown in FIG. 2F) nor the viability of the cell culture (estimated by FS/SS) was affected by the compound.

EC₅₀ values for inhibition of MHC-I downmodulation in HIV-1 infected SupT1 cells were generated by evaluating serial fold dilutions of the test compounds. FIG. 3 shows dose-response curves for the “R1”, “R4”, and “DM” values to monitor MHC-I downmodulation (FIGS. 3A, 3B, and 3C, respectively). Inhibitors were added after 24 h of infection and the extent of MHC-I downmodulation was monitored 24 h later (after 48 h of infection) after staining with a MHC-I specific antibody. Compound #56 displayed the highest potency (˜1 nM EC₅₀ for R1) and compound #100 the lowest (70 nM) among the inhibitors tested in this experiment. GraphPad Prism software was used to generate the EC₅₀ values at which the HIV-induced downmodulation activity was reduced by 50% using a non-linear four-parameter curve fit of the data. FIG. 3A also shows the selectivity indices (SI) obtained by dividing the compound-induced toxicity (CC₅₀ values), determined in parallel with uninfected cells using the XTT cell viability assay, by the EC₅₀ value (R1). In summary, these results demonstrate the ability of a small-molecule to potently block the downmodulation of cell surface MHC-I induced by HIV-1 in SupT1 cells.

To determine whether the inhibitors of MHC-I downmodulation are specific for HIV infection a study to determine whether the compounds also interfere with expression of MHC-I in uninfected cells was undertaken. The effect of the same compounds was evaluated in SupT-1 cells treated with HIV-1 as shown before, but this time the levels of expression of MHC-I were obtained in GFP-negative cells (FIG. 4). These results showed that the inhibitors tested (compound #40, #53, #54, #56 and #100) increased the surface levels of MHC-I in uninfected cells as well. The increase was dose-dependent and compounds elevated MHC-I levels by as much as 70% (as compared to cells treated with vehicle alone).

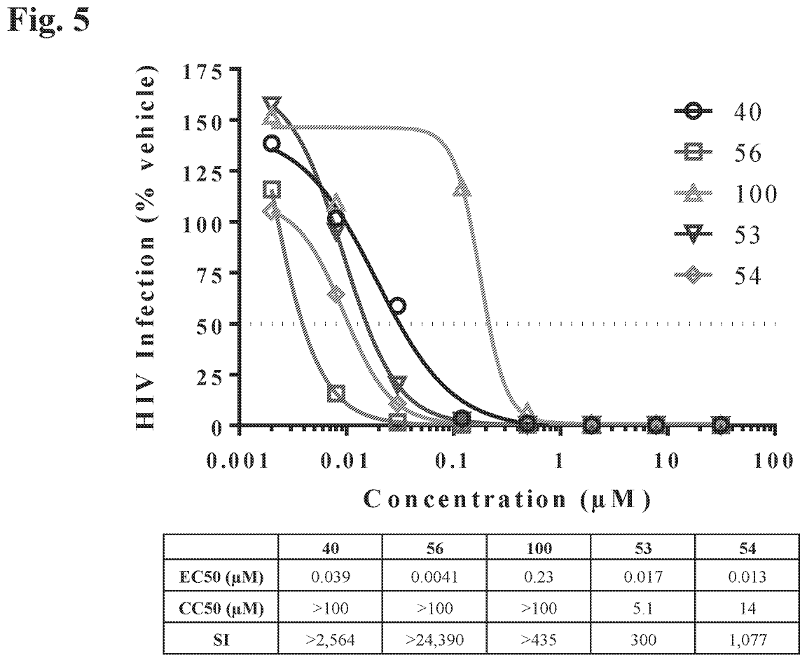

The ability of the compounds to interfere with HIV infection was evaluated in FIG. 5. Previously described experiments performed with SupT1 were done by incubating infected cells with compounds added 20 hours after infection. To determine whether the compounds also have anti-HIV activity HeLa-CD4-LTR-bgal reporter cells were used in which infection with HIV-1 results in expression of beta-galactosidase driven by the HIV-1 LTR promoter [Cortés, M. J.; Wong-Staal, F.; Lama, J. 2002. “Cell surface CD4 interferes with the infectivity of HIV-1 particles released from T cells.” J. Biol. Chem., 277(3), 1770-9]. Cells were preincubated with serial dilutions of compounds for 15 minutes and then infected with HIV-1 (LAI strain) for 48 h (FIG. 5). The extent of HIV-1 infection was revealed with a kit to detect beta-galactosidase levels in infected cells (Galacto-Star, ThermoFisher Scientific). The experiment revealed a potent dose-dependent inhibition of HIV-1 infection with the tested compounds, with EC₅₀ values varying from 4 nM to 230 nM. CC₅₀ values for compound-induced cytotoxicity evaluated in the same cells but uninfected are also shown in this figure. These results demonstrate that in addition to blocking MHC-I downmodulation in Supt1 cells, these inhibitors also interfere with HIV-1 infection using HeLa reporter cells. Similar HIV inhibition effects were observed when compounds were added to SupT1 immediately after infection (data not shown) rather than 20 h later, as shown in FIG. 2 and FIG. 3. The mechanism of HIV-1 inhibition mediated by these compounds is unknown but may be related to recent findings showing that HIV-1 Nef downmodulates other cell proteins including SERINC3 and SERINC5 [Usami Y, Wu Y., and Gottlinger, “SerinC3 and SerinC5 restrict HIV-1 infectivity and are counteracted by Nef.” Nature (2015), 526(7572):218-23]. SerinC3 and C₅ proteins interfere with HIV-1 infection when not removed by Nef. The inhibitors of MHC-I downmodulation may also affect the ability of Nef to downmodulate these proteins from the virons and thus result in reduced infectivity.

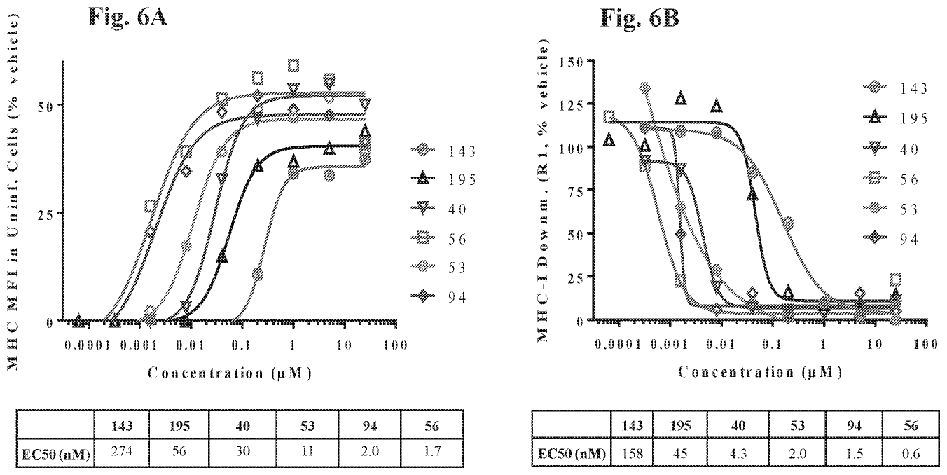

In some instances, the effect of small molecules in modulation of surface MHC-I was monitored in uninfected (rather than HIV-infected) Sup-T1 cells (FIG. 6A). In these experiments uninfected SupT1 cells were incubated with the compounds for 24 h and then the increase in MHC-I surface expression was monitored by staining cells with the biotinylated HLA-A2/28 antibody (One Lambda, clone BIH0037), washed and then incubated with streptavidin conjugated with APC (Streptavidin APC, EBiosciences, 17-4317) to reveal the anti-HLA-A2 bound antibody. After washing the cells again they were fixed with 0.5% paraformaldehyde and submitted to flow cytometry to determine the levels of MHC-I surface expression (APC fluorescence) in live cells (as estimated by FS/SS in these cells). Dose-response curves with increasing concentrations of compounds were generated as shown in FIG. 3, but this time the mean surface levels of MHC-I in live cells was indicated and compared with the levels of MHC-I observed in cells treated with vehicle alone. From these assays “mean-MHC-I” EC₅₀ values were generated to indicate the concentration of compound that augments the mean surface levels of MHC-I to 50% of the highest levels accomplished by the compound, as compared to cells treated with vehicle alone. Of note, the activity of the compounds in blocking downmodulation of MHC-I in HIV-infected SupT1cells (expressed as “R1% EC₅₀”, as shown in FIG. 6B) was found to correlate with the ability of the compounds to increase mean levels of MHC-I in uninfected cells “mean-MHC-I” EC₅₀, as shown in FIG. 6A. Therefore, both assays: inhibition of MHC-I downmodulation in HIV infected SupT1 cells, or increase in surface MHC-I in uninfected SupT1 cells, were indistinctively used to monitor the potency of the compounds to modulate surface expression of MHC-I in SupT1 cells.

Tables 3 and 4 show the activity utilizing the assay described in FIG. 3 as described in Example 2. The EC₅₀ for inhibition of HIV-induced MHC-I downmodulation is determined using the methods described in FIG. 3A using the R1 parameter.

Table 3, providing data from representative compounds of Table 1, and Table 4, providing data from representative compounds of Table 2, show the activity of selected compounds to block MHC-I downmodulation in SupT1 cells infected with HIV-1 as provided herein. Compound-induced toxicity (CC₅₀) is also shown.

TABLE 3

| | EC₅₀ | CC₅₀ | | EC₅₀ | CC₅₀ | | EC₅₀ | CC₅₀ |
|---|---|---|---|---|---|---|---|---|
| Cpd# | (nM)¹ | (μM)² | Cpd# | (nM) | (μM) | Cpd# | (nM) | (μM) |
| 1 | 120 | >90 | 55 | 210 | >125 | 111 | 180 | >125 |
| 2 | 2100 | >90 | 56 | 1 | >125 | 112 | 220 | >125 |
| 3 | 200 | >100 | 57 | 2 | >125 | 113 | 550 | >125 |
| 4 | 68 | >100 | 58 | 290 | >100 | 114 | 1200 | >125 |
| 5 | 550 | >100 | 59 | 860 | >100 | 115 | 1800 | >125 |
| 6 | 6300 | 36 | 60 | 3800 | >100 | 116 | 200 | >125 |
| 7 | >25000 | >100 | 61 | 2170 | >100 | 117 | 950 | >125 |
| 8 | 23000 | >100 | 62 | 480 | >100 | 118 | 2100 | >125 |
| 9 | 500 | >125 | 63 | 190 | >100 | 119 | 780 | >125 |
| 10 | >25000 | >100 | 64 | 570 | >100 | 120 | 4800 | >125 |
| 11 | >25000 | >100 | 65 | 880 | >125 | 121 | 3900 | >125 |
| 12 | >25000 | >100 | 67 | 2200 | >125 | 122 | 5000 | >125 |
| 13 | >25000 | >100 | 68 | 540 | >125 | 123 | 13 | >125 |
| 14 | >25000 | >100 | 69 | 5000 | >125 | 124 | 260 | >125 |
| 15 | >25000 | >100 | 70 | 890 | >125 | 125 | 880 | >125 |
| 16 | 10000 | >90 | 71 | 150 | >125 | 126 | 800 | >125 |
| 17 | 22000 | >125 | 73 | 210 | >100 | 127 | >25000 | >125 |
| 18 | 18000 | >125 | 74 | 1300 | >125 | 128 | >25000 | >125 |
| 19 | >30000 | >90 | 75 | 210 | 120 | 129 | >25000 | >125 |
| 20 | >30000 | >90 | 76 | 23000 | >125 | 130 | 380 | >125 |
| 21 | 560 | >100 | 77 | >25000 | >125 | 131 | 2600 | ~125 |
| 22 | 25000 | >100 | 78 | 17000 | >125 | 132 | 81 | >125 |
| 23 | 4400 | >100 | 79 | 5000 | >125 | 133 | 1500 | >100 |
| 24 | 4500 | >100 | 80 | 4600 | >125 | 134 | 11000 | >100 |
| 25 | 3600 | >100 | 81 | >25000 | >125 | 135 | 88 | >125 |
| 26 | >25000 | >100 | 82 | 5900 | >125 | 136 | 12000 | 123.54 |
| 27 | 1100 | >100 | 83 | 13000 | >125 | 137 | >25000 | 12 |
| 28 | >25000 | >100 | 84 | 890 | >100 | 138 | >25000 | >125 |
| 29 | >25000 | >100 | 85 | 380 | >100 | 139 | >25000 | >125 |
| 30 | 20000 | >125 | 86 | 1100 | >100 | 140 | 14000 | >125 |
| 31 | 210 | >100 | 87 | 580 | >100 | 141 | 1840 | >100 |
| 32 | 9 | >100 | 88 | 3250 | >100 | 142 | 5500 | >125 |
| 33 | 180 | >100 | 89 | 380 | >100 | 144 | 29000 | >90 |
| 34 | 1400 | 12 | 90 | >25000 | >100 | 145 | 91 | >90 |
| 35 | 1420 | >100 | 91 | 890 | >100 | 146 | 1690 | >10 |
| 36 | 23600 | >100 | 92 | 25000 | >100 | 147 | 90 | >90 |
| 37 | 2 | 0.15 | 93 | >25000 | >100 | 148 | 2000 | >100 |
| 38 | 6 | 0.25 | 94 | 1.5 | >125 | 149 | >25000 | >100 |
| 39 | 20 | >100 | 95 | 950 | >125 | 150 | >25000 | >90 |
| 40 | 8 | >100 | 96 | >25000 | >125 | 151 | >25000 | >90 |
| 41 | 20 | >125 | 97 | >25000 | >125 | 152 | >25000 | >90 |
| 42 | 7300 | >125 | 98 | 29000 | >90 | 153 | >25000 | >90 |
| 43 | 710 | >100 | 99 | 22000 | >125 | 154 | >25000 | >100 |
| 44 | 33 | >125 | 100 | 70 | 0.1 | 155 | >25000 | >90 |
| 45 | 510 | >100 | 101 | >25000 | >100 | 156 | >25000 | >90 |
| 46 | 59 | >125 | 102 | >25000 | >100 | 157 | >25000 | >90 |
| 47 | 5500 | >125 | 103 | 1480 | >100 | 158 | 13000 | >125 |
| 48 | 21000 | >100 | 104 | 1100 | >125 | 159 | 180 | >90 |
| 49 | 3100 | >125 | 105 | 390 | >125 | 160 | 280 | >125 |
| 50 | 14000 | >125 | 106 | 420 | >125 | 161 | 150 | >125 |
| 51 | 15 | >125 | 107 | 370 | >100 | 162 | 27 | 124 |
| 52 | 32 | 1 | 108 | 1370 | >100 | 163 | 370 | >125 |
| 53 | 3 | >125 | 109 | 2100 | >125 | | | |
| 54 | 8 | >125 | 110 | 4100 | >125 | | | |

TABLE 4

| | EC₅₀ | CC₅₀ | | EC₅₀ | CC₅₀ | | EC₅₀ | CC₅₀ |
|---|---|---|---|---|---|---|---|---|
| Cpd# | (nM)¹ | (μM)² | Cpd# | (nM) | (μM) | Cpd# | (nM) | (μM) |
| 66 | 3400 | >125 | 181 | 620 | >90 | 189 | >25000 | >100 |
| 72 | 1400 | >100 | 182 | 370 | 69 | 190 | >25000 | >100 |
| 143 | 410 | >100 | 183 | 4200 | >100 | 191 | >25000 | >100 |
| 175 | 1200 | 10 | 184 | 3300 | >90 | 192 | 1800 | >10 |
| 176 | 8900 | >100 | 185 | 200 | >90 | 193 | >25000 | >90 |
| 177 | 240 | >100 | 186 | 25000 | >100 | 194 | 1700 | 125 |
| 178 | >25000 | >25 | 187 | 25000 | >100 | 195 | 53 | >125 |
| 179 | >25000 | >100 | 188 | 10000 | >100 | 196 | >25000 | >100 |
| 180 | 28 | >100 | | | | | | |

¹EC₅₀ was calculated for inhibition of MHC-I downmodulation utilizing the R1 parameter. Values are given as percentage of samples treated with vehicle alone included in each experiment.
²CC₅₀: Concentrations reducing cell viability by 50%. Viability was measured using the XTT method with uninfected SupT1 cells incubated with compounds for 48 h. Values are compared to those observed with vehicle alone (0.1% DMSO).

In instances where the EC₅₀ of the compound was >25 μM, selected compounds were retested up to a concentration of 100 μM and their R1 value determined and compared to vehicle control. Under this setting, an R1 value lower than 100 is indicative of inhibition of MHC-I downmodulation above that observed with the vehicle control. Table 5, providing data for selected compounds of Table 1, and Table 6, providing data for selected compounds of Table 2, show the MHC-downmodulation observed in SupT1 cells infected with HIV-1 in the presence of selected compounds tested at 100 □M (“R1 @100 μM”) as provided herein. R1 values are given as percentage of vehicle controls. CC₅₀ values are also included.

TABLE 5

| | R1 @ | | | R1 @ | | | R1 @ | |
|---|---|---|---|---|---|---|---|---|
| | 100 | CC₅₀ | | 100 | CC₅₀ | | 100 | CC₅₀ |
| Cpd# | μM³ | (μM) | Cpd# | μM | (μM) | Cpd# | μM | (μM) |
| 7 | 34 | >100 | 28 | 80 | >100 | 149 | 27 | >100 |
| 10 | 46 | >100 | 29 | 65 | >100 | 150 | 47 | >90 |
| 11 | 48 | >100 | 77 | 21 | >125 | 151 | 51 | >90 |
| 12 | 51 | >100 | 81 | 67 | >125 | 152 | 54 | >90 |
| 13 | 63 | >100 | 90 | 31 | >100 | 153 | 39 | >90 |
| 14 | 18 | >100 | 93 | 65 | >100 | 154 | 44 | >100 |
| 15 | 14 | >100 | 96 | 32 | >125 | 155 | ≥100 | >90 |
| 19 | 20 | >90 | 97 | 5.4 | >125 | 156 | 40 | >90 |
| 20 | 56 | >90 | 101 | 40 | >100 | 157 | ≥100 | >90 |
| 26 | 53 | >100 | 102 | 20 | >100 | | | |

TABLE 6

| | R1 @ | | | R1 @ | | | R1 @ | |
|---|---|---|---|---|---|---|---|---|
| | 100 | CC₅₀ | | 100 | CC₅₀ | | 100 | CC₅₀ |
| Cpd# | μM³ | (μM) | Cpd# | μM | (μM) | Cpd# | μM | (μM) |
| 178 | 20 | >25 | 188 | 37 | >100 | 191 | 62 | >100 |
| 186 | 18 | >100 | 189 | 39 | >100 | 193 | 58 | >90 |
| 187 | 45 | >100 | 190 | 34 | >100 | 196 | 31 | >100 |

³Based on R1 value for MHC-1 downmodulation at 100 μM as a % of the vehicle control of infected cells. Values of 100 indicate no inhibition of HIV-induced downmodulation of MHC-I.

Table 7 show the activity of selected compounds utilizing the assay in which the increase of MHC-I surface expression in uninfected SupT1 cells was evaluated, as shown in FIG. 6A. The EC₅₀ for MHC-I increased surface expression was determined using the methods described in Example 2 and illustrated in FIG. 6A.

TABLE 7

| | EC₅₀ | CC₅₀ | | EC₅₀ | CC₅₀ | | EC₅₀ | CC₅₀ |
|---|---|---|---|---|---|---|---|---|
| Cpd# | (nM)¹ | (μM)² | Cpd# | (nM) | (μM) | Cpd# | (nM) | (μM) |
| 53 | 12 | >125 | 166 | 200 | >125 | 171 | 6200 | >125 |
| 54 | 9 | >125 | 167 | 470 | >125 | 172 | 8400 | >125 |
| 56 | 6 | >125 | 168 | >25,000 | >125 | 173 | 2500 | >125 |
| 164 | 26 | >125 | 169 | >25,000 | >125 | 174 | 2500 | >125 |
| 165 | 1000 | >125 | 170 | 1000 | >125 | | | |

What is claimed is:

1. A compound, or a pharmaceutically acceptable salt thereof, of Formula I: 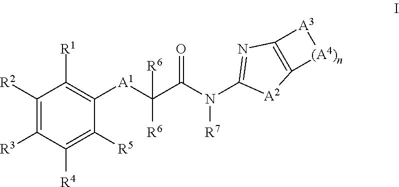
wherein:
R¹ and R² are taken together to form a ring which is selected from the group consisting of heteroaryl optionally substituted with 1-4 R⁸ and heterocyclyl optionally substituted with 1-4 R⁹;
with the proviso that 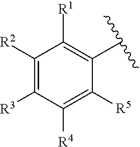
does not form 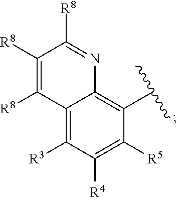
R³, R⁴, and R⁵ are independently selected from the group consisting of H, halide, unsubstituted —(C₁₋₆ alkyl), unsubstituted —(C₁₋₄ haloalkyl), —OR¹⁰, and —CN;
each R⁶ is independently selected from the group consisting of H, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein;
R⁷ is selected from the group consisting of H, unsubstituted —(C₁₋₉ alkyl), unsubstituted —(C₃₋₉ alkenyl), unsubstituted —(C₃₋₉ alkynyl), unsubstituted —(C₁₋₉ haloalkyl), —(C₁₋₄ alkylene)C(═O)OR¹¹, —(C₁₋₄ alkylene)C(═O)N(R¹²)₂, —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein;
each R⁸ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR¹⁰, and —CN;
each R⁹ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
each R¹⁰ is independently selected from the group consisting of H, unsubstituted —(C₁₋₉ alkyl), unsubstituted —(C₃₋₉ alkenyl), unsubstituted —(C₃₋₉ alkynyl), and unsubstituted —(C₁₋₉ haloalkyl);
each R¹¹ is independently selected from the group consisting of H and unsubstituted —(C₁₋₉ alkyl);
each R¹² is independently selected from the group consisting of H and substituted or unsubstituted —(C₁₋₉ alkyl);
each R¹³ is independently selected from the group consisting of halide, unsubstituted —(C₁₋₉ alkyl), unsubstituted —(C₁₋₉ haloalkyl), and —OR¹⁰;
each R¹⁴ is independently selected from the group consisting of halide, unsubstituted —(C₁₋₉ alkyl), unsubstituted —(C₁₋₉ haloalkyl), and —OR¹⁰;
each R¹⁵ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁶ is independently selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
A¹ is selected from the group consisting of —C(R¹⁵)₂—, O, and S;
A² is selected from the group consisting of O and S;
A³ is selected from the group consisting of —C(R¹⁶)₂—, O and S;
each A⁴ is independently selected from the group consisting of —C(R¹⁶)₂—, O and S;
with the proviso that no more than two A³ and A⁴ are O or S and that two O or S are never adjacent;
n is 1, 2, 3, or 4;
each p independently is 0 or 1; and
with the proviso that Formula I is not a structure selected from the group consisting of:

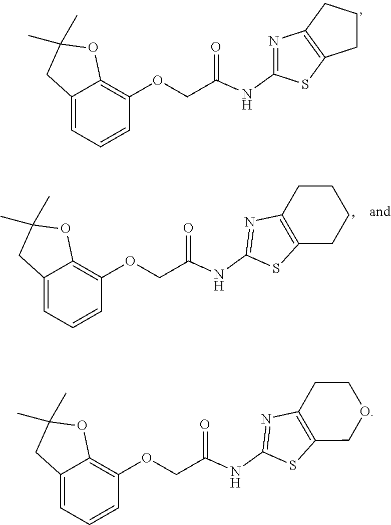 [SMILES: CC1(C)Cc2cccc(OCC(=O)Nc3nc4c(s3)CCC4)c2O1 ; CC1(C)Cc2cccc(OCC(=O)Nc3nc4c(s3)CCCC4)c2O1 ; CC1(C)Cc2cccc(OCC(=O)Nc3nc4c(s3)COCC4)c2O1]

2. The compound of claim 1, wherein each R⁶ is H.

3. The compound of any one of claims 1-2, wherein R¹ and R² are taken together to form a heteroaryl optionally substituted with 1-4 R⁸.

4. The compound of any one of claims 1-3, wherein R¹ and R² are taken together to form an unsubstituted heteroaryl.

5. The compound of any one of claims 1-4, wherein R¹ and R² are taken together with the phenyl ring to form

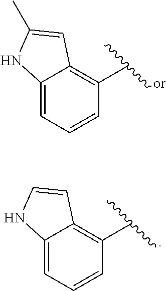 [SMILES: *c1cccc2[nH]c(C)cc12 ; *c1cccc2[nH]ccc12]

6. The compound of any one of claims 1-5, wherein R¹ and R² are taken together to form a heterocyclyl optionally substituted with 1-4 R⁹.

7. The compound of any one of claims 1-6, wherein R¹ and R² are taken together to form a heterocyclyl substituted with 1-2 methyls.

8. The compound of any one of claims 1-7, wherein R¹ and R² are taken together with the phenyl ring to form

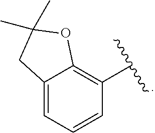 [SMILES: *c1cccc2c1OC(C)(C)C2]

9. The compound of any one of claims 1-8, wherein A² is S.

10. The compound of any one of claims 1-9, wherein A³ and all A⁴ are —C(R¹⁶)₂—.

11. The compound of any one of claims 1-10, wherein R¹⁶ is H.

12. The compound of any one of claims 1-11, wherein n is 2.

13. The compound of any one of claims 1-12, wherein n is 3.

14. The compound of any one of claims 1-13, wherein n is 4.

15. The compound of any of claims 1-14, wherein the compound of Formula I is selected from the group consisting of: 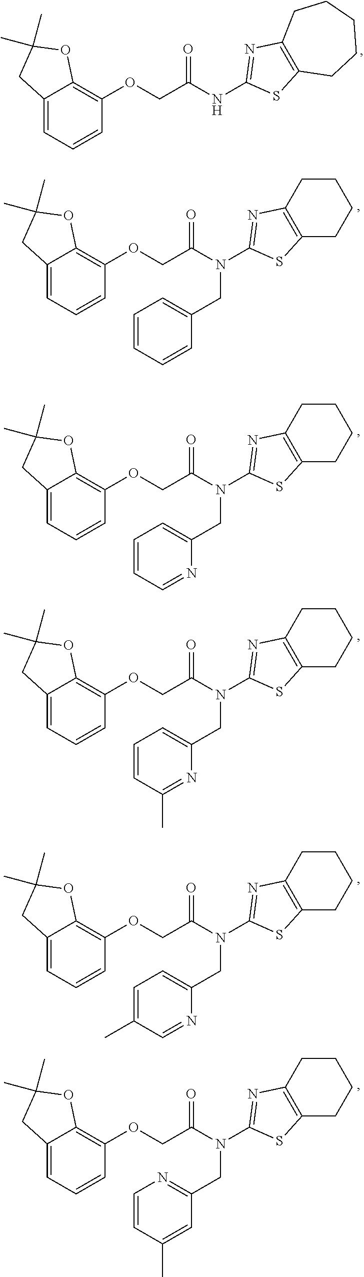 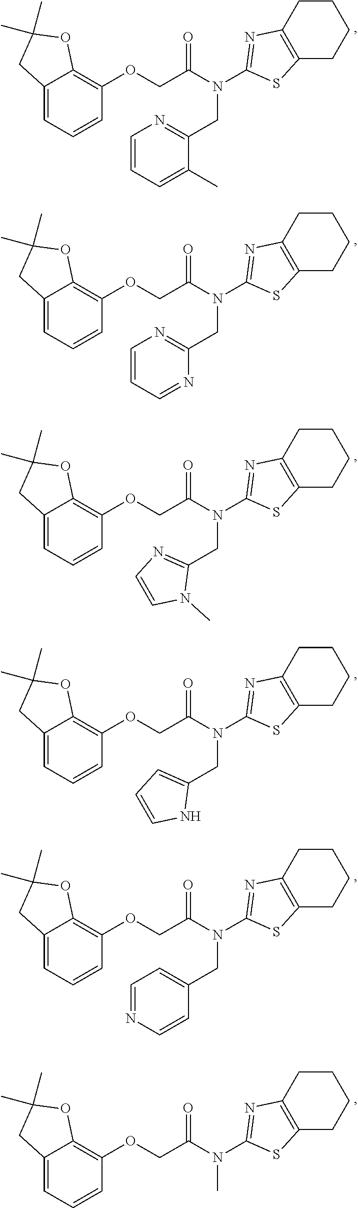 [SMILES: Cc1cccnc1CN(C(=O)COc1cccc2c1OC(C)(C)C2)c1nc2c(s1)CCCC2 ; CC1(C)Cc2cccc(OCC(=O)N(Cc3ncccn3)c3nc4c(s3)CCCC4)c2O1 ; Cn1ccnc1CN(C(=O)COc1cccc2c1OC(C)(C)C2)c1nc2c(s1)CCCC2 ; CC1(C)Cc2cccc(OCC(=O)N(Cc3ccc[nH]3)c3nc4c(s3)CCCC4)c2O1 ; CC1(C)Cc2cccc(OCC(=O)N(Cc3ccncc3)c3nc4c(s3)CCCC4)c2O1 ; CN(C(=O)COc1cccc2c1OC(C)(C)C2)c1nc2c(s1)CCCC2] 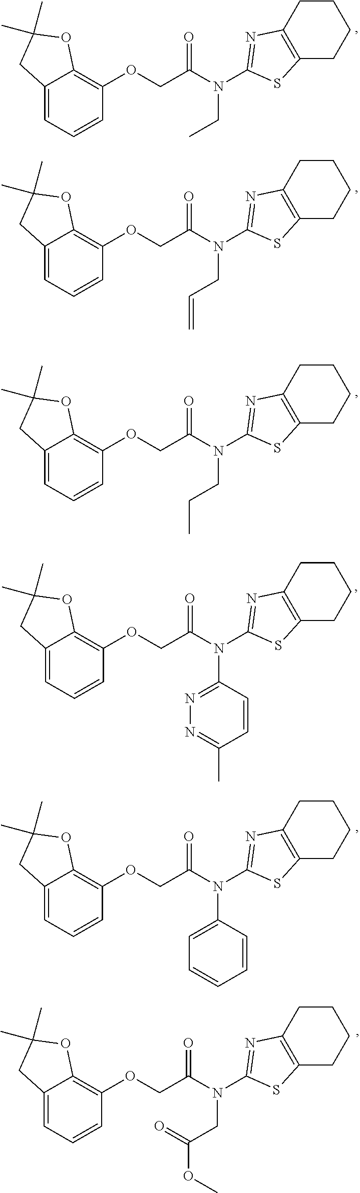 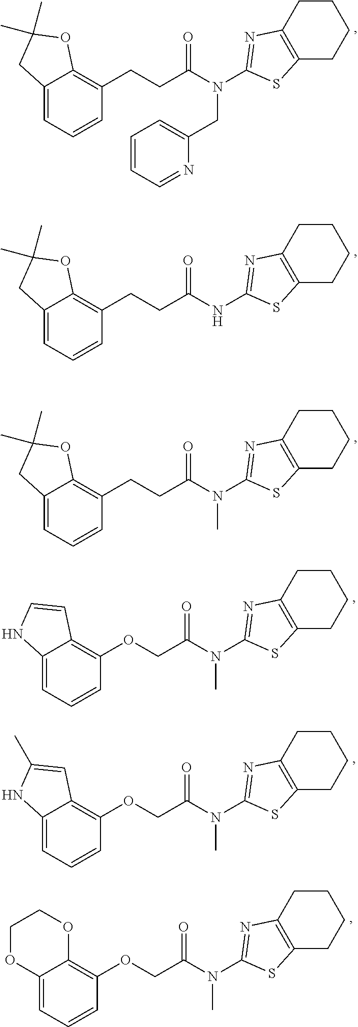 [SMILES: CC1(C)Cc2cccc(CCC(=O)N(Cc3ccccn3)c3nc4c(s3)CCCC4)c2O1 ; CC1(C)Cc2cccc(CCC(=O)Nc3nc4c(s3)CCCC4)c2O1 ; CN(C(=O)CCc1cccc2c1OC(C)(C)C2)c1nc2c(s1)CCCC2 ; CN(C(=O)COc1cccc2[nH]ccc12)c1nc2c(s1)CCCC2 ; Cc1cc2c(OCC(=O)N(C)c3nc4c(s3)CCCC4)cccc2[nH]1 ; CN(C(=O)COc1cccc2c1OCCO2)c1nc2c(s1)CCCC2] 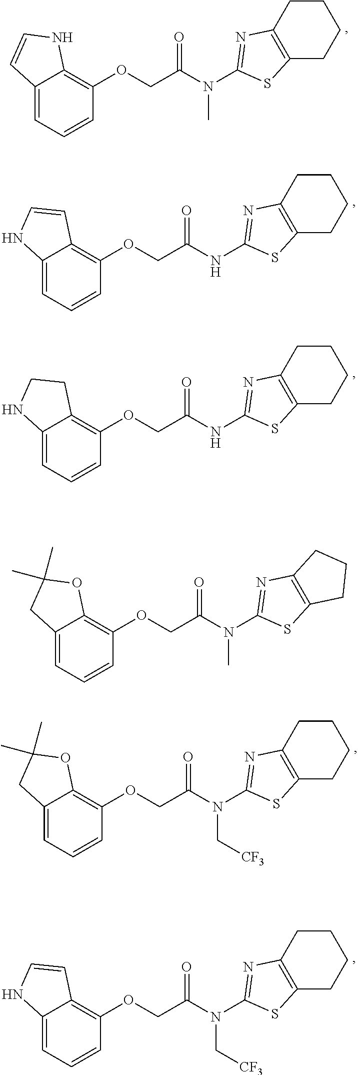 [SMILES: CN(C(=O)COc1cccc2cc[nH]c12)c1nc2c(s1)CCCC2 ; O=C(COc1cccc2[nH]ccc12)Nc1nc2c(s1)CCCC2 ; O=C(COc1cccc2c1CCN2)Nc1nc2c(s1)CCCC2 ; CN(C(=O)COc1cccc2c1OC(C)(C)C2)c1nc2c(s1)CCC2 ; CC1(C)Cc2cccc(OCC(=O)N(CC(F)(F)F)c3nc4c(s3)CCCC4)c2O1 ; O=C(COc1cccc2[nH]ccc12)N(CC(F)(F)F)c1nc2c(s1)CCCC2] 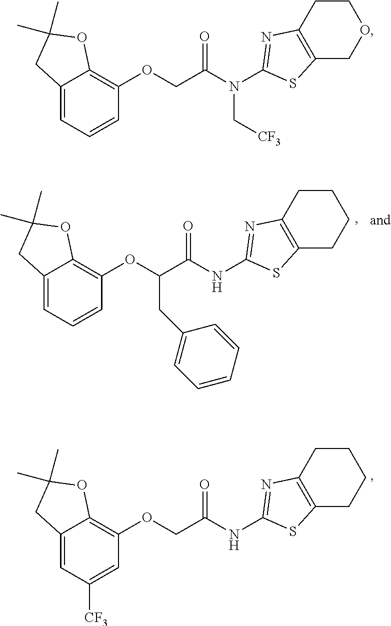 [SMILES: CC1(C)Cc2cccc(OCC(=O)N(CC(F)(F)F)c3nc4c(s3)COCC4)c2O1 ; CC1(C)Cc2cccc(OC(Cc3ccccc3)C(=O)Nc3nc4c(s3)CCCC4)c2O1 ; CC1(C)Cc2cc(C(F)(F)F)cc(OCC(=O)Nc3nc4c(s3)CCCC4)c2O1]
or a pharmaceutically acceptable salt thereof.

16. A compound, or a pharmaceutically acceptable salt thereof, of Formula II: 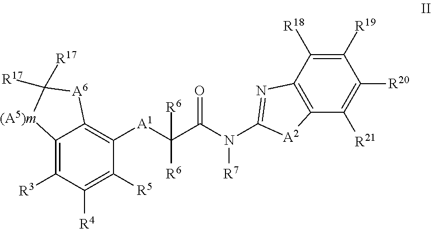
wherein:
R³, R⁴, and R⁵ are independently selected from the group consisting of H, halide, unsubstituted —(C₁₋₆ alkyl), unsubstituted —(C₁₋₄ haloalkyl), —OR¹⁰, and —CN;
each R⁶ is independently selected from the group consisting of H, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
R⁷ is selected from the group consisting of H, unsubstituted —(C₁₋₉ alkyl), unsubstituted —(C₃₋₉ alkenyl), unsubstituted —(C₃₋₉ alkynyl), unsubstituted —(C₁₋₉ haloalkyl), —(C₁₋₄ alkylene)C(═O)OR¹¹, —(C₁₋₄ alkylene)C(═O)N(R¹²)₂, —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein;
each R¹⁰ is independently selected from the group consisting of H, unsubstituted —(C₁₋₉ alkyl), unsubstituted —(C₃₋₉ alkenyl), unsubstituted —(C₃₋₉ alkynyl), and unsubstituted —(C₁₋₉ haloalkyl);
R¹¹ is selected from the group consisting of H and unsubstituted —(C₁₋₉ alkyl);
each R¹² is independently selected from the group consisting of H and substituted or unsubstituted —(C₁₋₉ alkyl);
each R¹³ is independently selected from the group consisting of halide, unsubstituted —(C₁₋₉ alkyl), unsubstituted —(C₁₋₉ haloalkyl), and —OR¹⁰;
each R¹⁴ is independently selected from the group consisting of halide, unsubstituted —(C₁₋₉ alkyl), unsubstituted —(C₁₋₉ haloalkyl), and —OR¹⁰;
each R¹⁵ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁶ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁷ is independently selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R¹⁸ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR¹⁰, —(C₁₋₄ alkylene)ₚheteroaryl optionally substituted with 1-4 R²², —(C₁₋₄ alkylene)ₚaryl optionally substituted with 1-5 R²³, and —CN; wherein each —(C₁₋₄ alkylene) is, independently, optionally substituted with one or more substituents as defined herein;
R¹⁹ is selected from the group consisting of H, halide, unsubstituted —(C₁₋₉ alkyl), and unsubstituted —(C₁₋₆ haloalkyl);
R²⁰ is selected from the group consisting of H, halide, unsubstituted —(C₁₋₉ alkyl), and unsubstituted —(C₁₋₆ haloalkyl);
R²¹ is selected from the group consisting of H, halide, unsubstituted —(C₁₋₉ alkyl), and unsubstituted —(C₁₋₆ haloalkyl);
each R²² is independently selected from the group consisting of halide, unsubstituted —(C₁₋₉ alkyl), unsubstituted —(C₁₋₉ haloalkyl), and —CN;
each R²³ is independently selected from the group consisting of halide, unsubstituted —(C₁₋₉ alkyl), unsubstituted —(C₁₋₉ haloalkyl), and —CN;
A¹ is selected from the group consisting of —C(R¹⁵)₂—, O, and S;
A² is selected from the group consisting of O and S;
each A⁵ is independently selected from the group consisting of —C(R¹⁶)₂—, —N(R¹⁰)—, O, and S;
with the proviso that two A⁵ are not both N, O or S;
A⁶ is selected from the group consisting of —C(R¹⁶)₂—, —N(R¹⁰)—, O, and S;
with the proviso that A⁵ and A⁶ cannot both be C(R¹⁶)₂;
m is 1 or 2;
each p independently is 0 or 1; and
with the proviso that Formula II is not a structure selected from the group consisting of:

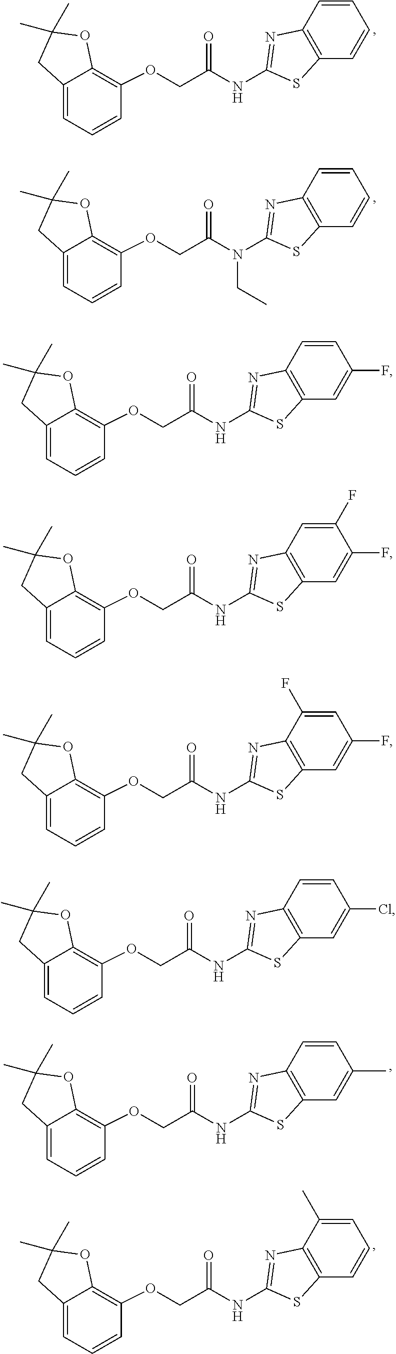

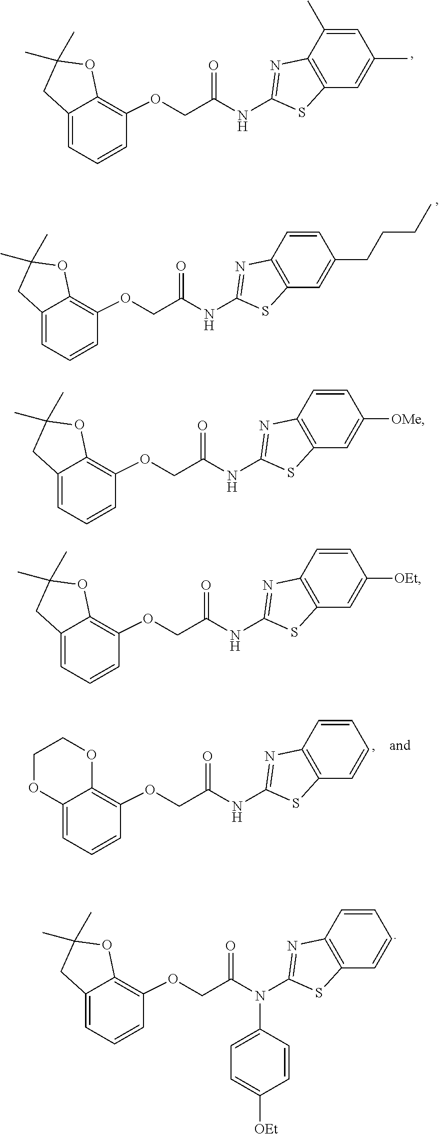

17. A compound, or a pharmaceutically acceptable salt thereof, of Formula II: 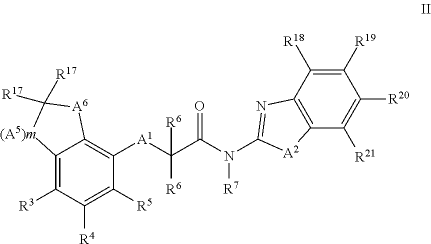
wherein:
R³, R⁴, and R⁵ are independently selected from the group consisting of H, halide, unsubstituted —(C₁₋₆ alkyl), unsubstituted —(C₁₋₄ haloalkyl), —OR¹⁰, and —CN;
each R⁶ is independently selected from the group consisting of H, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
R⁷ is selected from the group consisting of H, unsubstituted —(C₁₋₉ alkyl), unsubstituted —(C₃₋₉ alkenyl), unsubstituted —(C₃₋₉ alkynyl), unsubstituted —(C₁₋₉ haloalkyl), —(C₁₋₄ alkylene)C(═O)OR¹¹, —(C₁₋₄ alkylene)C(═O)N(R¹²)₂, —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein;
each R¹⁰ is independently selected from the group consisting of H, unsubstituted —(C₁₋₉ alkyl), unsubstituted —(C₃₋₉ alkenyl), unsubstituted —(C₃₋₉ alkynyl), and unsubstituted —(C₁₋₉ haloalkyl);
R¹¹ is selected from the group consisting of H and unsubstituted —(C₁₋₉ alkyl);
each R¹² is independently selected from the group consisting of H and substituted or unsubstituted —(C₁₋₉ alkyl);
each R¹³ is independently selected from the group consisting of halide, unsubstituted —(C₁₋₉ alkyl), unsubstituted —(C₁₋₉ haloalkyl), and —OR¹⁰;
each R¹⁴ is independently selected from the group consisting of halide, unsubstituted —(C₁₋₉ alkyl), unsubstituted —(C₁₋₉ haloalkyl), and —OR¹⁰;
each R¹⁵ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁶ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁷ is independently selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R¹⁸ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR¹⁰, —(C₁₋₄ alkylene)ₚheteroaryl optionally substituted with 1-4 R²², —(C₁₋₄ alkylene)ₚaryl optionally substituted with 1-5 R²³, and —CN; wherein each —(C₁₋₄ alkylene) is, independently, optionally substituted with one or more substituents as defined herein;
with the proviso that when A¹ is O or S, both R⁶ are H and R⁷ is selected from the group consisting of H, unsubstituted —(C₁₋₄ alkyl), and -aryl optionally substituted with —OR¹⁰; then R¹⁸ is not selected from the group consisting of H, halide, and unsubstituted —(C₁₋₄ alkyl);
R¹⁹ is selected from the group consisting of H, halide, unsubstituted —(C₁₋₉ alkyl), and unsubstituted —(C₁₋₆ haloalkyl);
R²⁰ is selected from the group consisting of H, halide, unsubstituted —(C₁₋₉ alkyl), and unsubstituted —(C₁₋₆ haloalkyl);
R²¹ is selected from the group consisting of H, halide, unsubstituted —(C₁₋₉ alkyl), and unsubstituted —(C₁₋₆ haloalkyl);
each R²² is independently selected from the group consisting of halide, unsubstituted —(C₁₋₉ alkyl), unsubstituted —(C₁₋₉ haloalkyl), and —CN;
each R²³ is independently selected from the group consisting of halide, unsubstituted —(C₁₋₉ alkyl), unsubstituted —(C₁₋₉ haloalkyl), and —CN;
A^{L} is selected from the group consisting of —C(R¹⁵)₂—, O, and S;
A² is selected from the group consisting of 0 and S;
each A⁵ is independently selected from the group consisting of —C(R¹⁶)₂—, —N(R¹⁰)—, O, and S;
with the proviso that two A⁵ are not both N, O or S;
A⁶ is selected from the group consisting of —C(R¹⁶)₂—, —N(R¹⁰)—, O, and S;
with the proviso that A⁵ and A⁶ cannot both be C(R¹⁶)₂;
m is 1 or 2; and
each p independently is 0 or 1.

18. The compound of any one of claims 16-17, wherein each R⁶ is H.

19. The compound of any one of claims 16-18, wherein m is 1.

20. The compound of any one of claims 16-19, wherein A⁵ and A⁶ are —O—.

21. The compound of any one of claims 16-20, wherein A⁵ is —C(R¹⁶)₂— and A⁶ is —O—.

22. The compound of any one of claims 16-21, wherein A⁵ is —O— and A⁶ is —C(R¹⁶)₂—.

23. The compound of any one of claims 16-22, wherein R¹⁶ is H.

24. The compound of any one of claims 16-23, wherein each R¹⁷ is independently selected from the group consisting of H, halide, and unsubstituted —(C₁₋₃ alkyl).

25. The compound of any one of claims 16-24, wherein each R¹⁷ is unsubstituted —(C₁₋₂ alkyl).

26. The compound of any one of claims 16-25, wherein each R¹⁷ is methyl.

27. The compound of any one of claims 16-26, wherein A² is S.

28. The compound of any one of claims 16-27, wherein R¹⁸ is selected from the group consisting of unsubstituted —(C₁₋₃ haloalkyl), —O(C₁₋₃ alkyl), -heteroaryl optionally substituted with 1-4 R²²,-aryl optionally substituted with 1-5 R²³, and —CN;

29. The compound of any one of claims 16-28, wherein R¹⁸ is selected from the group consisting of —CF₃, —OMe, -heteroaryl optionally substituted with 1 R²², phenyl optionally substituted with 1-2 R²³, and —CN.

30. The compound of any one of claims 16-29, wherein R²² is selected from the group consisting of methyl and halide.

31. The compound of any one of claims 16-30, wherein R²³ is selected from the group consisting of methyl, halide, and —CN.

32. The compound of any one of claims 16-31, wherein R¹⁹, R²⁰, and R²¹ are independently selected from the group consisting of H and halide.

33. The compound of any of claims 16-32, wherein the compound of Formula II is selected from the group consisting of: 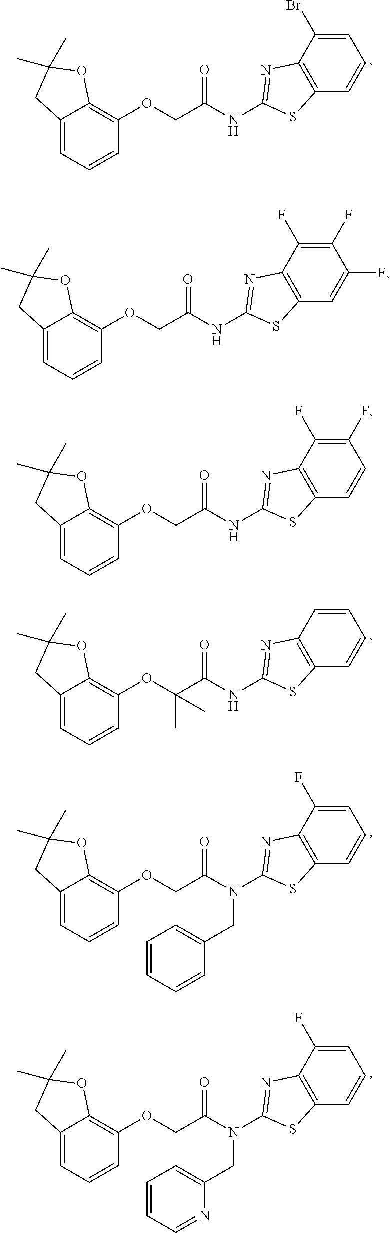 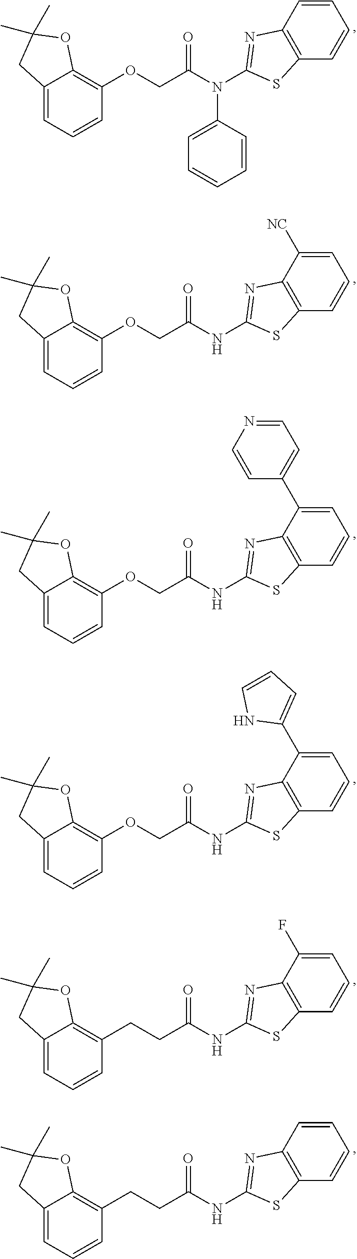 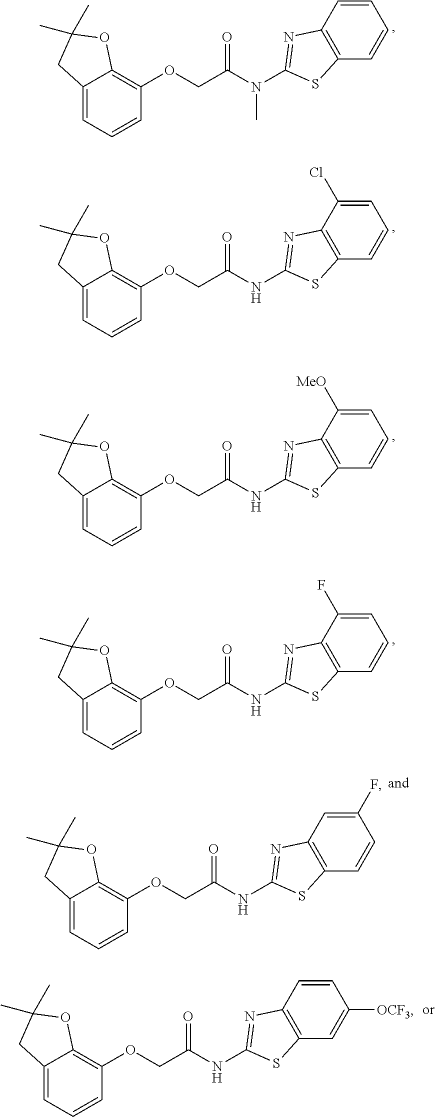 [SMILES: CN(C(=O)COc1cccc2c1OC(C)(C)C2)c1nc2ccccc2s1 ; CC1(C)Cc2cccc(OCC(=O)Nc3nc4c(Cl)cccc4s3)c2O1 ; COc1cccc2sc(NC(=O)COc3cccc4c3OC(C)(C)C4)nc12 ; CC1(C)Cc2cccc(OCC(=O)Nc3nc4c(F)cccc4s3)c2O1 ; CC1(C)Cc2cccc(OCC(=O)Nc3nc4cc(F)ccc4s3)c2O1 ; CC1(C)Cc2cccc(OCC(=O)Nc3nc4ccc(OC(F)(F)F)cc4s3)c2O1]
a pharmaceutically acceptable salt thereof.

34. A compound, or a pharmaceutically acceptable salt thereof, of Formula III: 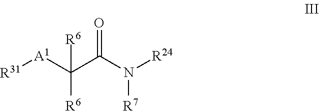
wherein:
R³, R⁴, and R⁵ are independently selected from the group consisting of H, halide, unsubstituted —(C₁₋₆ alkyl), unsubstituted —(C₁₋₄ haloalkyl), —OR¹⁰, and —CN;
each R⁶ is independently selected from the group consisting of H, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein;
R⁷ is selected from the group consisting of H, unsubstituted —(C₁₋₉ alkyl), unsubstituted —(C₃₋₉ alkenyl), unsubstituted —(C₃₋₉ alkynyl), unsubstituted —(C₁₋₉ haloalkyl), —(C₁₋₄ alkylene)C(═O)OR¹¹, —(C₁₋₄ alkylene)C(═O)N(R¹²)₂, —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein;
each R¹⁰ is independently selected from the group consisting of H, unsubstituted —(C₁₋₉ alkyl), unsubstituted —(C₃₋₉ alkenyl), unsubstituted —(C₃₋₉ alkynyl), and unsubstituted —(C₁₋₉ haloalkyl);
R¹¹ is selected from the group consisting of H and unsubstituted —(C₁₋₉ alkyl);
each R¹² is independently selected from the group consisting of H and substituted or unsubstituted —(C₁₋₉ alkyl);
each R¹³ is independently selected from the group consisting of halide, unsubstituted —(C₁₋₉ alkyl), unsubstituted —(C₁₋₉ haloalkyl), and —OR¹⁰;
each R¹⁴ is independently selected from the group consisting of halide, unsubstituted —(C₁₋₉ alkyl), unsubstituted —(C₁₋₉ haloalkyl), and —OR¹⁰;
each R¹⁵ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁶ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁷ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R²⁴ is selected from the group consisting of a 6-membered heteroaryl optionally substituted with 1-4 R²⁵, 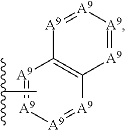
wherein 1-4 A⁹ are N; and 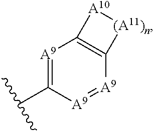
wherein 1-2 A⁹ are N;
each R²⁵ is independently selected from the group consisting of halide, —CN, —OR²⁸, unsubstituted —(C₁₋₆ alkyl), unsubstituted —(C₂₋₆ alkenyl), unsubstituted —(C₂₋₆ alkynyl), unsubstituted —(C₁₋₆ haloalkyl), and carbocyclyl optionally substituted with 1-10 R²⁹;
each R²⁶ is independently selected from the group consisting of H, halide, —CN, —OR²⁸, unsubstituted —(C₁₋₆ alkyl), unsubstituted —(C₂₋₆ alkenyl), unsubstituted —(C₂₋₆ alkynyl), unsubstituted —(C₁₋₆ haloalkyl), and carbocyclyl optionally substituted with 1-10 R²⁹;
each R²⁷ is independently selected from the group consisting of H, halide, —CN, —OR²⁸, unsubstituted —(C₁₋₆ alkyl), unsubstituted —(C₂₋₆ alkenyl), unsubstituted —(C₂₋₆ alkynyl), unsubstituted —(C₁₋₆ haloalkyl), and carbocyclyl optionally substituted with 1-10 R²⁹;
each R²⁸ is independently selected from the group consisting of unsubstituted —(C₁₋₉ alkyl), unsubstituted —(C₃₋₉ alkenyl), unsubstituted —(C₃₋₉ alkynyl), and unsubstituted —(C₁₋₉ haloalkyl);
each R²⁹ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
R³¹ is selected from the group consisting of H and unsubstituted —(C₁₋₉ alkyl);
R³¹ is selected from the group consisting of 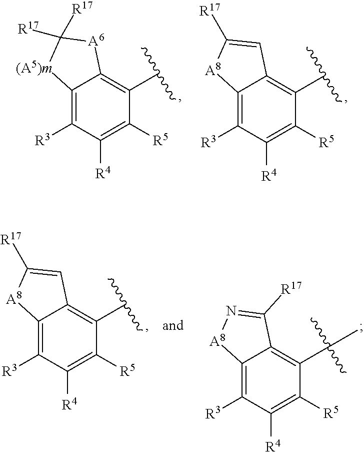
A¹ is selected from the group consisting of —C(R¹⁵)₂—, O, and S;
each A⁵ is independently selected from the group consisting of —C(R¹⁶)₂—, —N(R¹)—, O, and S;
with the proviso that two A⁵ are not both N, O or S;
A⁶ is selected from the group consisting of —C(R¹⁶)₂—, —N(R¹⁰)—, O, and S;
with the proviso that A⁵ and A⁶ cannot both be C(R¹⁶)₂;
A⁷ is selected from the group consisting of —N(R³⁰)—, O and S;
A⁶ is selected from the group consisting of —NH—, O and S;
each A⁹ is independently selected from the group consisting of N and —C(R²⁶)—;
A¹⁰ is selected from the group consisting of —C(R²⁷)₂—, O, and S;
each A¹¹ is independently selected from the group consisting of —C(R²⁷)₂—, O, and S;
with the proviso that no more than two A¹⁰ or A¹¹ are O or S and that two O or S are never adjacent;
n is 1, 2, 3, or 4;
m is 1 or 2;
each p independently is 0 or 1; and
with the proviso that Formula III is not a structure selected from the group consisting of:

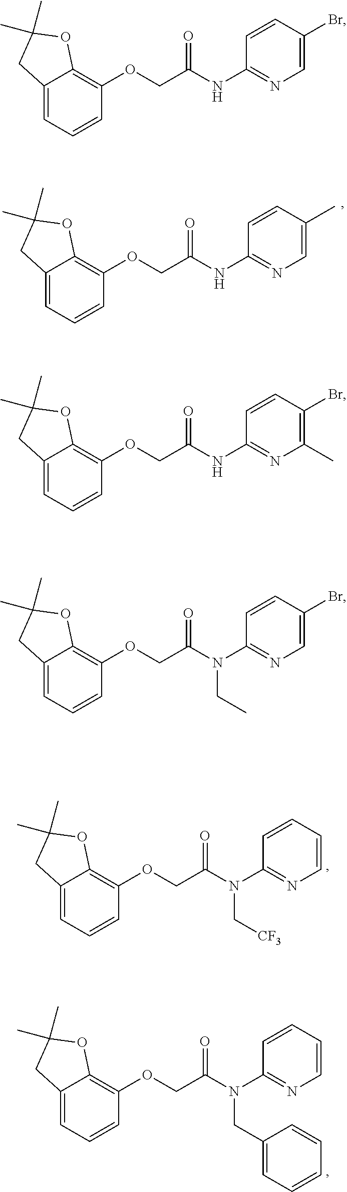

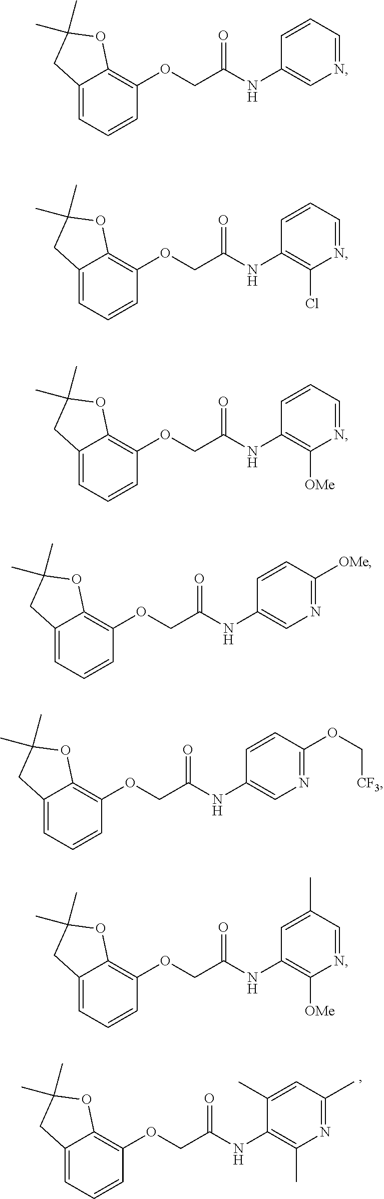

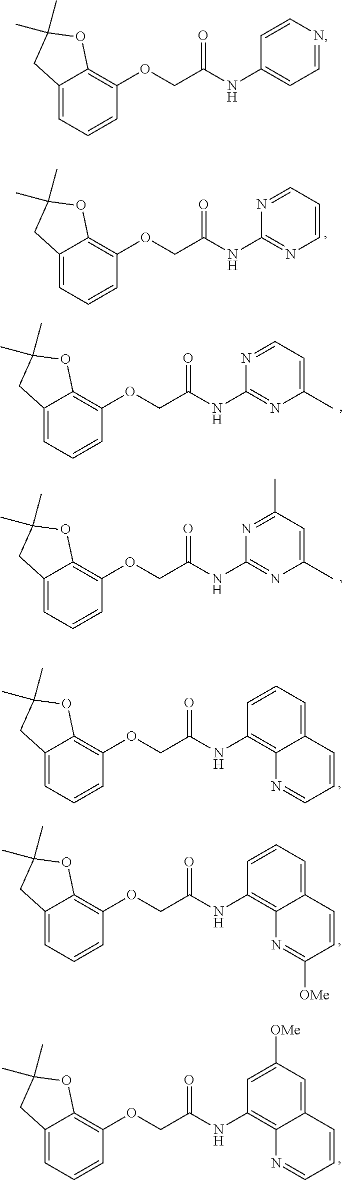

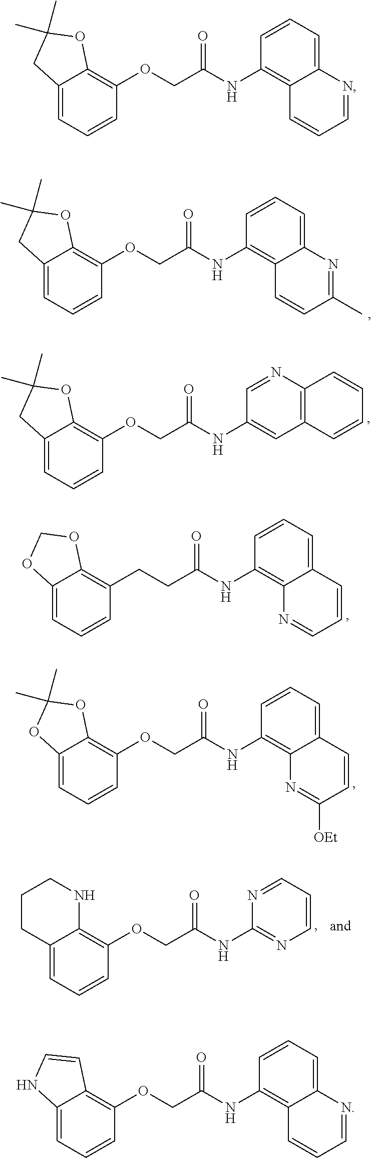

35. A compound, or a pharmaceutically acceptable salt thereof, of Formula III: 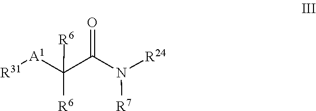
wherein:
R³, R⁴, and R⁵ are independently selected from the group consisting of H, halide, unsubstituted —(C₁₋₆ alkyl), unsubstituted —(C₁₋₄ haloalkyl), —OR¹⁰, and —CN;
each R⁶ is independently selected from the group consisting of H, unsubstituted —(C₁₋₆ alkyl), unsubstituted —(C₂₋₆ alkenyl), unsubstituted —(C₂₋₆ alkynyl), unsubstituted —(C₁-6 haloalkyl), —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein;
R⁷ is selected from the group consisting of H, unsubstituted —(C₁₋₉ alkyl), unsubstituted —(C₃₋₉ alkenyl), unsubstituted —(C₃₋₉ alkynyl), unsubstituted —(C₁₋₉ haloalkyl), —(C₁₋₄ alkylene)C(═O)OR¹¹, —(C₁₋₄ alkylene)C(═O)N(R¹²)₂, —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein;
each R¹⁰ is independently selected from the group consisting of H, unsubstituted —(C₁₋₉ alkyl), unsubstituted —(C₃₋₉ alkenyl), unsubstituted —(C₃₋₉ alkynyl), and unsubstituted —(C₁₋₉ haloalkyl);
R¹¹ is selected from the group consisting of H and unsubstituted —(C₁₋₉ alkyl);
each R¹² is independently selected from the group consisting of H and substituted or unsubstituted —(C₁₋₉ alkyl);
each R¹³ is independently selected from the group consisting of halide, unsubstituted —(C₁₋₉ alkyl), unsubstituted —(C₁₋₉ haloalkyl), and —OR¹⁰;
each R¹⁴ is independently selected from the group consisting of halide, unsubstituted —(C₁₋₉ alkyl), unsubstituted —(C₁₋₉ haloalkyl), and —OR¹⁰;
each R¹⁵ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁶ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁷ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R²⁴ is selected from the group consisting of a 6-membered heteroaryl optionally substituted with 1-4 R²⁵, 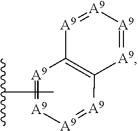
wherein 1-4 A⁹ are N; and 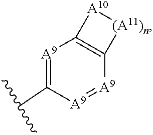
wherein 1-2 A⁹ are N;
with the proviso that when R³¹ is 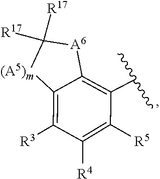
A¹ is O or S, both R⁶ are H, and R⁷ is selected from the group consisting of H, unsubstituted —(C₁₋₃ alkyl), unsubstituted —(C₁₋₃ haloalkyl), —(CH₂)aryl optionally substituted with 1-5 halides, then R²⁴ is not selected from the group consisting of pyridine, 2-pyrimidine, and quinoline;
with the proviso that when R³¹ is 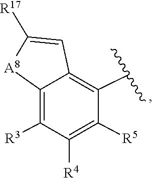
A¹ is O or S, both R⁶ are H, and R⁷ is H, then R²⁴ is not quinoline;
each R²⁵ is independently selected from the group consisting of halide, —CN, —OR²⁸, unsubstituted —(C₁₋₆ alkyl), unsubstituted —(C₂₋₆ alkenyl), unsubstituted —(C₂₋₆ alkynyl), unsubstituted —(C₁₋₆ haloalkyl), and carbocyclyl optionally substituted with 1-10 R²⁹;
each R²⁶ is independently selected from the group consisting of H, halide, —CN, —OR²⁸, unsubstituted —(C₁₋₆ alkyl), unsubstituted —(C₂₋₆ alkenyl), unsubstituted —(C₂₋₆ alkynyl), unsubstituted —(C₁₋₆ haloalkyl), and carbocyclyl optionally substituted with 1-10 R²⁹;
each R²⁷ is independently selected from the group consisting of H, halide, —CN, —OR²⁸, unsubstituted —(C₁₋₆ alkyl), unsubstituted —(C₂₋₆ alkenyl), unsubstituted —(C₂₋₆ alkynyl), unsubstituted —(C₁₋₆ haloalkyl), and carbocyclyl optionally substituted with 1-10 R²⁹;
each R²⁸ is independently selected from the group consisting of unsubstituted —(C₁₋₉ alkyl), unsubstituted —(C₃₋₉ alkenyl), unsubstituted —(C₃₋₉ alkynyl), and unsubstituted —(C₁₋₉ haloalkyl);
each R²⁹ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
R³¹ is selected from the group consisting of H and unsubstituted —(C₁₋₉ alkyl);
R³¹ is selected from the group consisting of 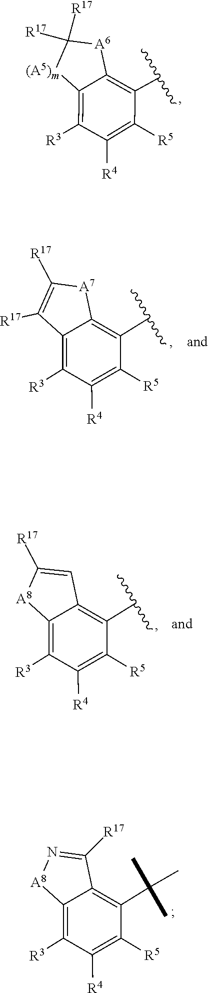
A¹ is selected from the group consisting of —C(R¹⁵)₂—, O, and S;
each A⁵ is independently selected from the group consisting of —C(R¹⁶)₂—, —N(R¹⁰)—, O, and S;
with the proviso that two A⁵ are not both N, O or S;
A⁶ is selected from the group consisting of —C(R¹⁶)₂—, —N(R¹⁰)—, O, and S;
with the proviso that A⁵ and A⁶ cannot both be C(R¹⁶)₂;
A⁷ is selected from the group consisting of —N(R³⁰)—, O and S;
A⁸ is selected from the group consisting of —NH—, O and S;
each A⁹ is independently selected from the group consisting of N and —C(R²⁶)—;
A¹⁰ is selected from the group consisting of —C(R²⁷)₂—, O, and S;
each A¹¹ is independently selected from the group consisting of —C(R²⁷)₂—, O, and S;
with the proviso that no more than two A¹⁰ or A¹¹ are O or S and that two O or S are never adjacent;
n is 1, 2, 3, or 4;
m is 1 or 2;
each p independently is 0 or 1; and
with the proviso that Formula III is not a structure selected from the group consisting of:

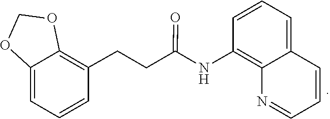 [SMILES: O=C(CCc1cccc2c1OCO2)Nc1cccc2cccnc12]

36. The compound of any one of claims 34-35, wherein each R⁶ is H.

37. The compound of any one of claims 34-36, wherein R³¹ is

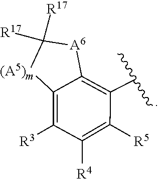

38. The compound of any one of claims 34-37, wherein m is 1.

39. The compound of any one of claims 34-38, wherein A⁵ and A⁶ are —O—.

40. The compound of any one of claims 34-39, wherein A⁵ is —C(R¹⁶)₂— and A⁶ is —O—.

41. The compound of any one of claims 34-40, wherein A⁵ is —O— and A⁶ is —C(R¹⁶)₂—.

42. The compound of any one of claims 34-41, wherein R¹⁶ is H.

43. The compound of any one of claims 34-42, wherein each R¹⁷ is independently selected from the group consisting of H, halide, and unsubstituted —(C₁₋₃ alkyl).

44. The compound of any one of claims 34-43, wherein each R¹⁷ is unsubstituted —(C₁₋₂ alkyl).

45. The compound of any one of claims 34-44, wherein each R¹⁷ is methyl.

46. The compound of any one of claims 34-45, wherein R³¹ is

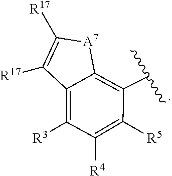

47. The compound of any one of claims 34-46, wherein A⁷ is NH.

48. The compound of any one of claims 34-47, wherein each R¹⁷ is H.

49. The compound of any one of claims 34-48, wherein R³¹ is

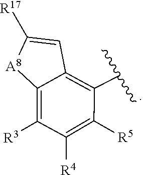

50. The compound of any one of claims 34-49, wherein A⁸ is NH.

51. The compound of any one of claims 34-50, wherein R²⁴ is a 6-membered heteroaryl optionally substituted with 1-4 R²⁵.

52. The compound of any one of claims 34-51, wherein R²⁴ is selected from the group consisting of 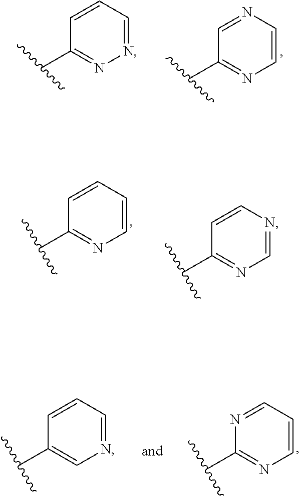 [SMILES: *c1cccnn1 ; *c1cnccn1 ; *c1ccccn1 ; *c1ccncn1 ; *c1cccnc1 ; *c1ncccn1]
each optionally substituted with 1-2 R²⁵.

53. The compound of any one of claims 34-52, wherein each R²⁵ is independently selected from the group consisting of halide, —CN, —OMe, unsubstituted —(C₁₋₃ alkyl), unsubstituted —(C₁₋₂ haloalkyl), and an unsubstituted 3-4 membered carbocyclyl.

54. The compound of any one of claims 34-53, wherein R²⁴ is

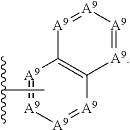

55. The compound of any one of claims 34-54, wherein R²⁴ is selected from the group consisting of

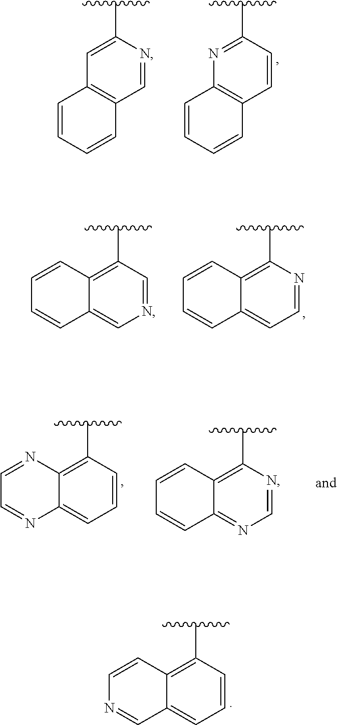

56. The compound of any one of claims 34-55, wherein each R²⁶ is independently selected from the group consisting of halide, —CN, —OMe, unsubstituted —(C₁₋₃ alkyl), and unsubstituted —(C₁₋₂ haloalkyl).

57. The compound of any one of claims 34-56, wherein R²⁴ is

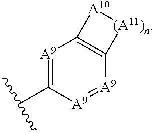

58. The compound of any of claims 34-57, wherein the compound of Formula III is selected from the group consisting of: 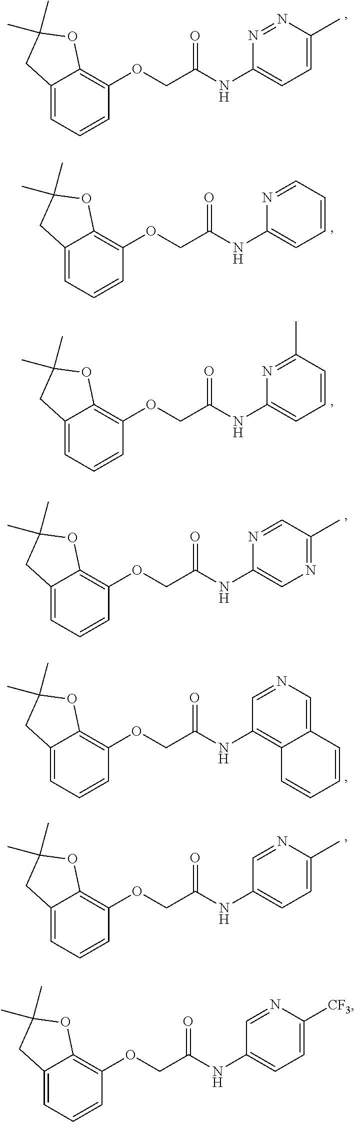 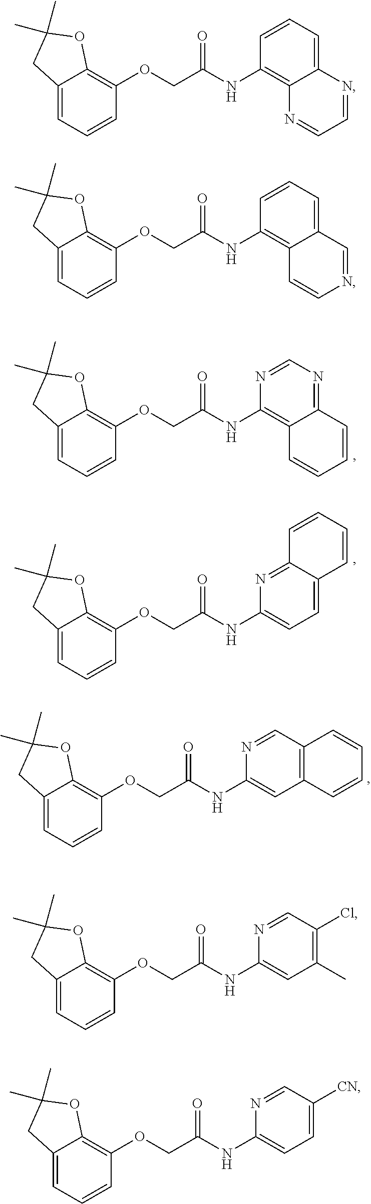 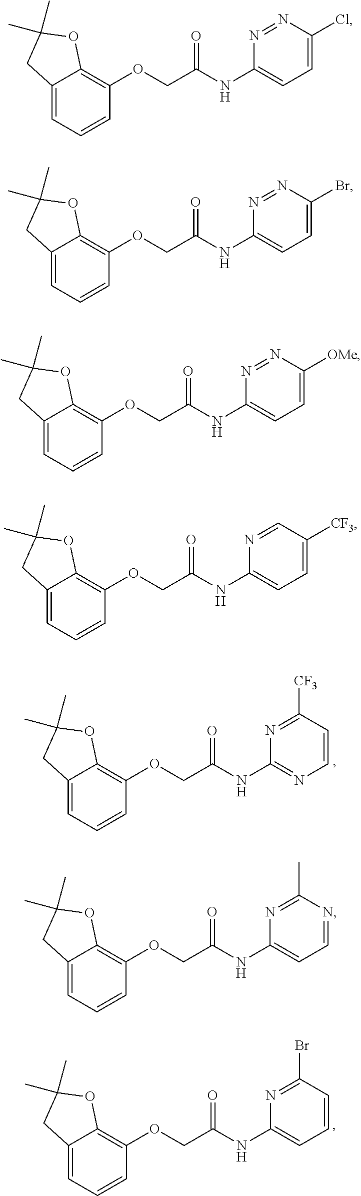 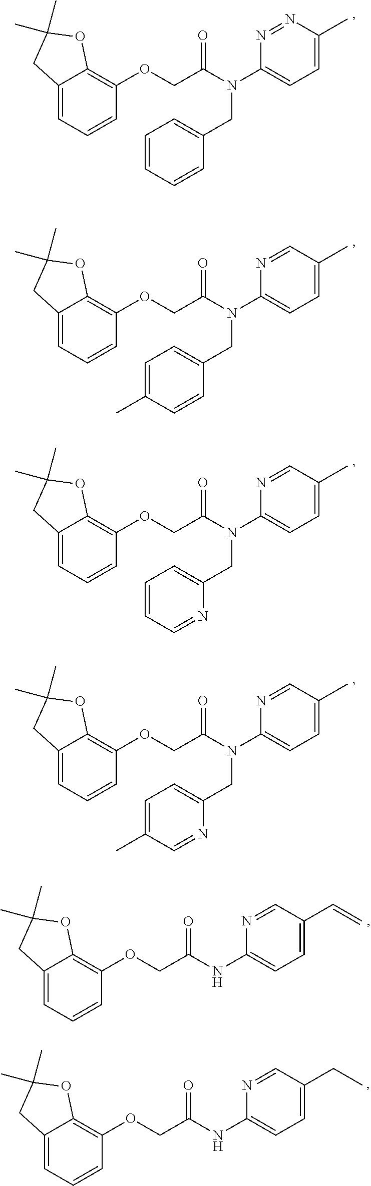 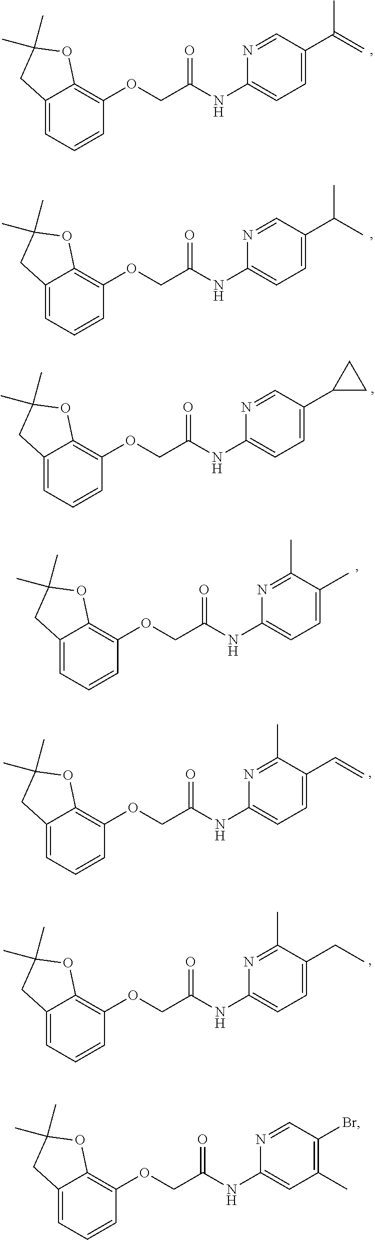 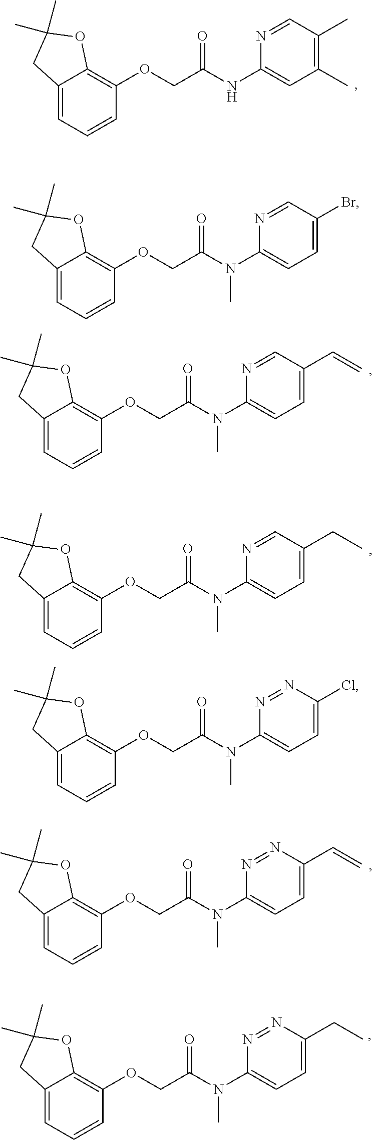 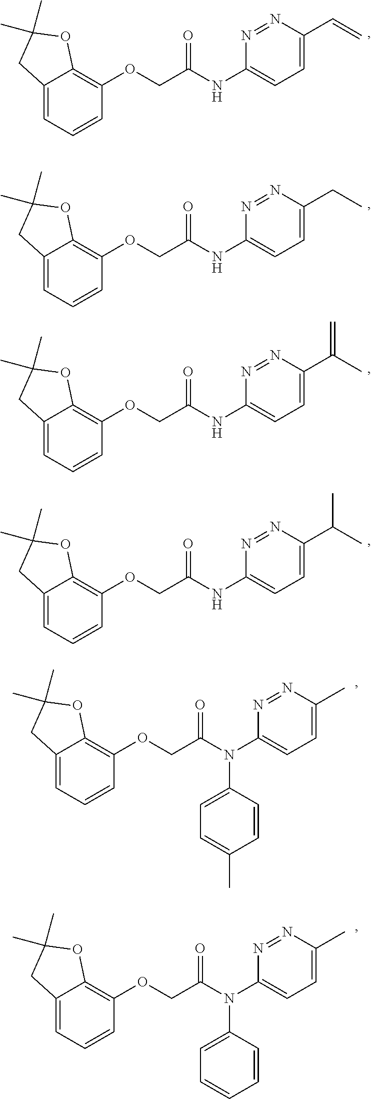 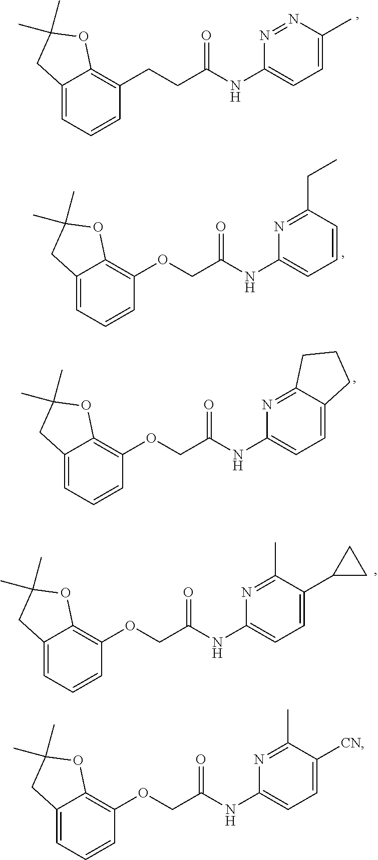 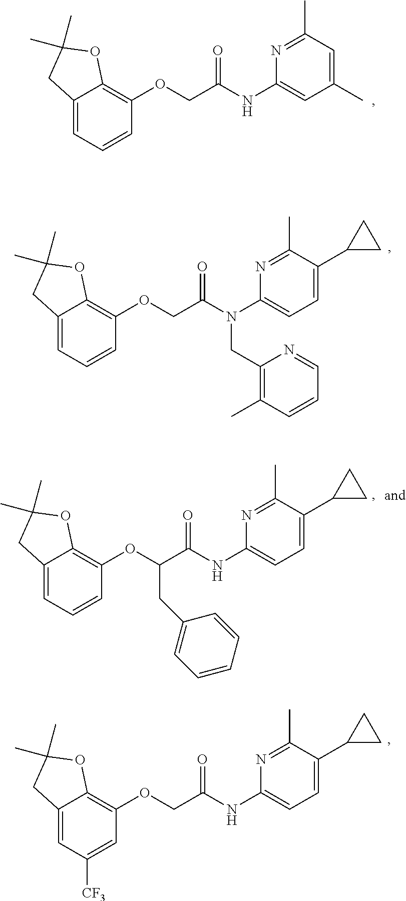
or a pharmaceutically acceptable salt thereof.

59. A compound, or a pharmaceutically acceptable salt thereof, of Formula IV: 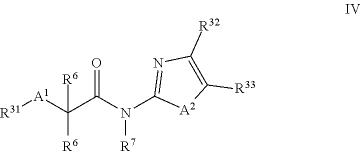
wherein:
R³, R⁴, and R⁵ are independently selected from the group consisting of H, halide, unsubstituted —(C₁₋₆ alkyl), unsubstituted —(C₁₋₄ haloalkyl), —OR¹⁰, and —CN;
each R⁶ is independently selected from the group consisting of H, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
R⁷ is selected from the group consisting of H, unsubstituted —(C₁₋₉ alkyl), unsubstituted —(C₃₋₉ alkenyl), unsubstituted —(C₃₋₉ alkynyl), unsubstituted —(C₁₋₉ haloalkyl), —(C₁₋₄ alkylene)C(═O)OR¹¹, —(C₁₋₄ alkylene)C(═O)N(R¹²)₂, —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein;
each R¹⁰ is independently selected from the group consisting of H, unsubstituted —(C₁₋₉ alkyl), unsubstituted —(C₃₋₉ alkenyl), unsubstituted —(C₃₋₉ alkynyl), and unsubstituted —(C₁₋₉ haloalkyl);
R¹¹ is selected from the group consisting of H and unsubstituted —(C₁₋₉ alkyl);
each R¹² is independently selected from the group consisting of H and substituted or unsubstituted —(C₁₋₉ alkyl);
each R¹³ is independently selected from the group consisting of halide, unsubstituted —(C₁₋₉ alkyl), unsubstituted —(C₁₋₉ haloalkyl), and —OR¹⁰;
each R¹⁴ is independently selected from the group consisting of halide, unsubstituted —(C₁₋₉ alkyl), unsubstituted —(C₁₋₉ haloalkyl), and —OR¹⁰;
each R¹⁵ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁶ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁷ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R³⁰ is selected from the group consisting of H and unsubstituted —(C₁₋₉ alkyl);
R³¹ is selected from the group consisting of 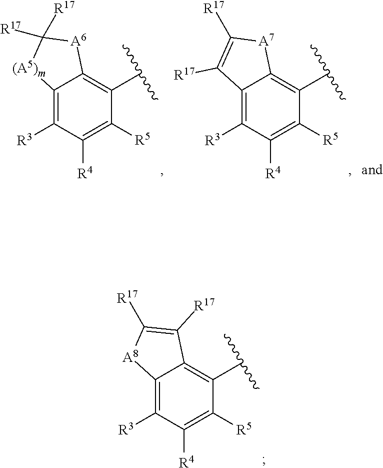
R³² is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -carbocyclyl optionally substituted with 1-10 R³⁴, —C(═O)R³⁵, C(═O)NHR³⁶, and —CN;
R³³ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -carbocyclyl optionally substituted with 1-10 R³⁷, —C(═O)R³⁸, —C(═O)NHR³⁹, and —CN;
each R³⁴ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
R³⁵ is -heterocyclyl optionally substituted with 1-10 R⁴⁰;
R³⁶ is selected from the group consisting of —(C₁₋₄ alkylene)heterocyclyl optionally substituted with 1-10 R⁴¹ and —(C₁₋₄ alkylene)heteroaryl optionally substituted with 1-10 R⁴²; wherein each —(C₁₋₄ alkylene) is, independently, optionally substituted with one or more substituents as defined herein;
each R³⁷ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
R³⁸ is -heterocyclyl optionally substituted with 1-10 R⁴⁴;
R³⁹ is selected from the group consisting of —(C₁₋₄ alkylene)heterocyclyl optionally substituted with 1-10 R⁴⁵ and —(C₁₋₄ alkylene)heteroaryl optionally substituted with 1-10 R⁴⁶; wherein each —(C₁₋₄ alkylene) is, independently, optionally substituted with one or more substituents as defined herein;
each R⁴⁰ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(C₁₋₄ alkylene)ₚOR⁴³; wherein —(C₁₋₄ alkylene) is optionally substituted with one or more substituents as defined herein;
each R⁴¹ is independently selected from the group consisting of halide, unsubstituted 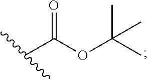 [SMILES: *C(=O)OC(C)(C)C]
—(C₁₋₉ alkyl), and O each R⁴² is independently selected from the group consisting of halide and unsubstituted —(C₁₋₉ alkyl);
each R⁴³ is independently selected from the group consisting of H and unsubstituted —(C₁₋₉ alkyl);
each R⁴⁴ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(C₁₋₄ alkylene)ₚOR⁴⁷; wherein —(C₁₋₄ alkylene) is optionally substituted with one or more substituents as defined herein;
each R⁴⁵ is independently selected from the group consisting of halide, unsubstituted —(C₁₋₉ alkyl), and 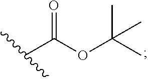 [SMILES: *C(=O)OC(C)(C)C]
each R⁴⁶ is independently selected from the group consisting of halide and unsubstituted —(C₁₋₉ alkyl);
each R⁴⁷ is independently selected from the group consisting of H and unsubstituted —(C₁₋₉ alkyl);
A¹ is selected from the group consisting of —C(R¹⁵)₂—, O, and S;
A² is selected from the group consisting of O and S;
each A⁵ is independently selected from the group consisting of —C(R¹⁶)₂—, —N(R¹⁰)—, O, and S;
with the proviso that two A⁵ are not both N, O or S;
A⁶ is selected from the group consisting of —C(R¹⁶)₂—, —N(R¹⁰)—, O, and S;
with the proviso that A⁵ and A⁶ cannot both be C(R¹⁶)₂;
A⁷ is selected from the group consisting of —N(R³⁰)—, O and S;
A⁸ is selected from the group consisting of —N(R³⁰)—, O and S;
m is 1 or 2;
each p independently is 0 or 1; and
with the proviso that Formula IV is not a structure selected from the group consisting of:

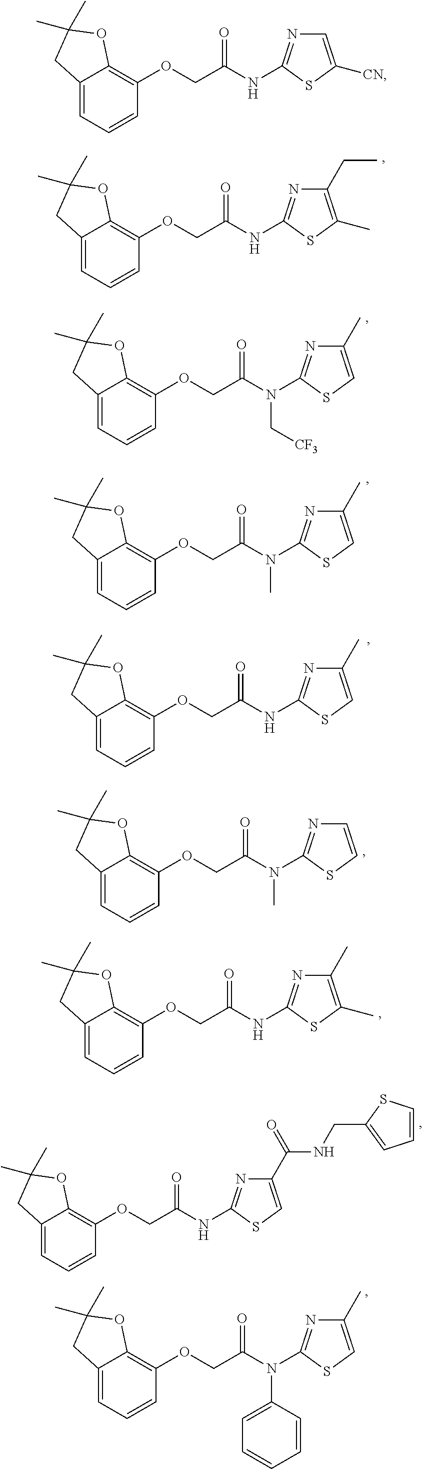

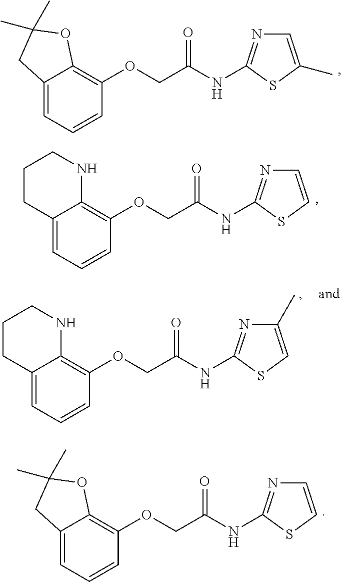

60. A compound, or a pharmaceutically acceptable salt thereof, of Formula IV: 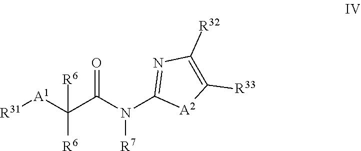
wherein:
R³, R⁴, and R⁵ are independently selected from the group consisting of H, halide, unsubstituted —(C₁₋₆ alkyl), unsubstituted —(C₁₋₄ haloalkyl), —OR¹⁰, and —CN;
each R⁶ is independently selected from the group consisting of H, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
R⁷ is selected from the group consisting of H, unsubstituted —(C₁₋₉ alkyl), unsubstituted —(C₃₋₉ alkenyl), unsubstituted —(C₃₋₉ alkynyl), unsubstituted —(C₁₋₉ haloalkyl), —(C₁₋₄ alkylene)C(═O)OR¹¹, —(C₁₋₄ alkylene)C(═O)N(R¹²)₂, —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein;
each R¹⁰ is independently selected from the group consisting of H, unsubstituted —(C₁₋₉ alkyl), unsubstituted —(C₃₋₉ alkenyl), unsubstituted —(C₃₋₉ alkynyl), and unsubstituted —(C₁₋₉ haloalkyl);
R¹¹ is selected from the group consisting of H and unsubstituted —(C₁₋₉ alkyl);
each R¹² is independently selected from the group consisting of H and substituted or unsubstituted —(C₁₋₉ alkyl);
each R¹³ is independently selected from the group consisting of halide, unsubstituted —(C₁₋₉ alkyl), unsubstituted —(C₁₋₉ haloalkyl), and —OR¹⁰;
each R¹⁴ is independently selected from the group consisting of halide, unsubstituted —(C₁₋₉ alkyl), unsubstituted —(C₁₋₉ haloalkyl), and —OR¹⁰;
each R¹⁵ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁶ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁷ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R³⁰ is selected from the group consisting of H and unsubstituted —(C₁₋₉ alkyl);
R³¹ is selected from the group consisting of 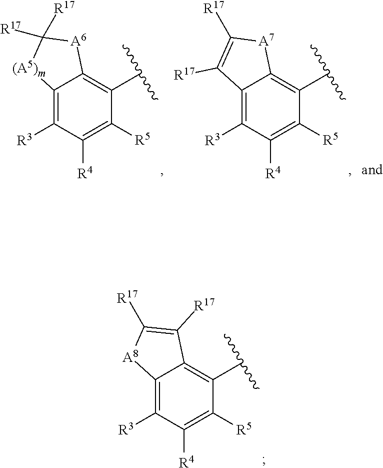
with the proviso that when R³¹ is 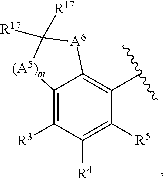
A¹ is O, m is 1 or 2, A⁵ is CH₂, both R⁶ are H and R⁷ is H, then R³² and R³³ are not selected from the group consisting of H, halide, unsubstituted —(C₁₋₃ alkyl), —CN, and 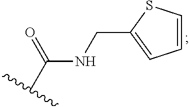 [SMILES: *C(=O)NCc1cccs1]
with the proviso that when R³¹ is R 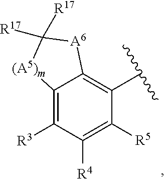
A¹ is O, m is 1, A⁵ is CH₂, both R⁶ are H and R⁷ is selected from the group consisting of unsubstituted —(C₁₋₂ alkyl), unsubstituted —(C₁₋₂ haloalkyl), -aryl optionally substituted with 1-5 R¹⁴, and R¹⁴ is halide or methyl, then R³² and R³³ are not selected from the group consisting of H, halide, and unsubstituted —(C₁₋₂ alkyl);
R³² is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -carbocyclyl optionally substituted with 1-10 R³⁴, —C(═O)R³⁵, C(═O)NHR³⁶, and —CN;
R³³ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -carbocyclyl optionally substituted with 1-10 R³⁷, —C(═O)R³⁸, —C(═O)NHR³⁹, and —CN;
each R³⁴ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
R³⁵ is -heterocyclyl optionally substituted with 1-10 R⁴⁰;
R³⁶ is selected from the group consisting of —(C₁₋₄ alkylene)heterocyclyl optionally substituted with 1-10 R⁴¹ and —(C₁₋₄ alkylene)heteroaryl optionally substituted with 1-10 R⁴²; wherein each —(C₁₋₄ alkylene) is, independently, optionally substituted with one or more substituents as defined herein;
each R³⁷ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
R³⁸ is -heterocyclyl optionally substituted with 1-10 R⁴⁴;
R³⁹ is selected from the group consisting of —(C₁₋₄ alkylene)heterocyclyl optionally substituted with 1-10 R⁴⁵ and —(C₁₋₄ alkylene)heteroaryl optionally substituted with 1-10 R⁴⁶; wherein each —(C₁₋₄ alkylene) is, independently, optionally substituted with one or more substituents as defined herein;
each R⁴¹ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(C₁₋₄ alkylene)ₚOR⁴³; wherein —(C₁₋₄ alkylene) is optionally substituted with one or more substituents as defined herein;
each R⁴¹ is independently selected from the group consisting of halide, unsubstituted —(C₁₋₉ alkyl), and 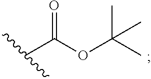 [SMILES: *C(=O)OC(C)(C)C]
each R⁴² is independently selected from the group consisting of halide and unsubstituted —(C₁₋₉ alkyl);
each R⁴³ is independently selected from the group consisting of H and unsubstituted —(C₁₋₉ alkyl);
each R⁴⁴ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(C₁₋₄ alkylene)ₚOR⁴⁷; wherein —(C₁₋₄ alkylene) is optionally substituted with one or more substituents as defined herein;
each R⁴⁵ is independently selected from the group consisting of halide, unsubstituted —(C₁₋₉ alkyl), and 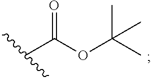 [SMILES: *C(=O)OC(C)(C)C]
each R⁴⁶ is independently selected from the group consisting of halide and unsubstituted —(C₁₋₉ alkyl);
each R⁴⁷ is independently selected from the group consisting of H and unsubstituted —(C₁₋₉ alkyl);
A¹ is selected from the group consisting of —C(R¹⁵)₂—, O, and S;
A² is selected from the group consisting of O and S;
each A⁵ is independently selected from the group consisting of —C(R¹⁶)₂—, —N(R¹⁰)—, O, and S;
with the proviso that two A⁵ are not both N, O or S;
A⁶ is selected from the group consisting of —C(R¹⁶)₂—, —N(R¹⁰)—, O, and S;
with the proviso that A⁵ and A⁶ cannot both be C(R¹⁶)₂;
A⁷ is selected from the group consisting of —N(R³⁰)—, O and S;
A⁸ is selected from the group consisting of —N(R³⁰)—, O and S;
m is 1 or 2; and
each p is 0 or 1.

61. The compound of any one of claims 59-60, wherein each R⁶ is H.

62. The compound of any one of claims 59-61, wherein R³¹ is

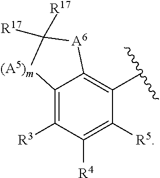

63. The compound of any one of claims 59-62, wherein m is 1.

64. The compound of any one of claims 59-63, wherein A⁵ and A⁶ are —O—.

65. The compound of any one of claims 59-64, wherein A⁵ is —C(R¹⁶)₂— and A⁶ is —O—.

66. The compound of any one of claims 59-65, wherein A⁵ is —O— and A⁶ is —C(R¹⁶)₂—.

67. The compound of any one of claims 59-66, wherein R¹⁶ is H.

68. The compound of any one of claims 59-67, wherein each R¹⁷ is independently selected from the group consisting of H, halide, and unsubstituted —(C₁₋₃ alkyl).

69. The compound of any one of claims 59-68, wherein each R¹⁷ is unsubstituted —(C₁₋₂ alkyl).

70. The compound of any one of claims 59-69, wherein each R¹⁷ is methyl.

71. The compound of any one of claims 59-70, wherein R³¹ is

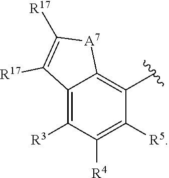

72. The compound of any one of claims 59-71, wherein A⁷ is NH.

73. The compound of any one of claims 59-72, wherein each R¹⁷ is H.

74. The compound of any one of claims 59-73, wherein R³¹ is NH

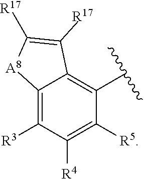

75. The compound of any one of claims 59-74, wherein A⁸ is NH.

76. The compound of any one of claims 59-75, wherein each R¹⁷ is H.

77. The compound of any one of claims 59-76, wherein A² is S.

78. The compound of any one of claims 59-77, wherein R³² is selected from the group consisting of H, halide, unsubstituted —(C₁₋₄ alkyl), unsubstituted —(C₁₋₃ haloalkyl), unsubstituted 3-4-membered carbocyclyl, —C(═O)R³⁵, —C(═O)NHR³⁶, and —CN.

79. The compound of any one of claims 59-78, wherein R³⁶ is selected from the group consisting of —(CH₂)heterocyclyl optionally substituted with 1-2 R⁴¹ and —(CH₂)heteroaryl optionally substituted with 1-2 R⁴².

80. The compound of any one of claims 59-79, wherein R³³ is selected from the group consisting of H, halide, methyl, —CF₃, and —CN.

81. The compound of any of claims 59-80, wherein the compound of Formula IV is selected from the group consisting of: 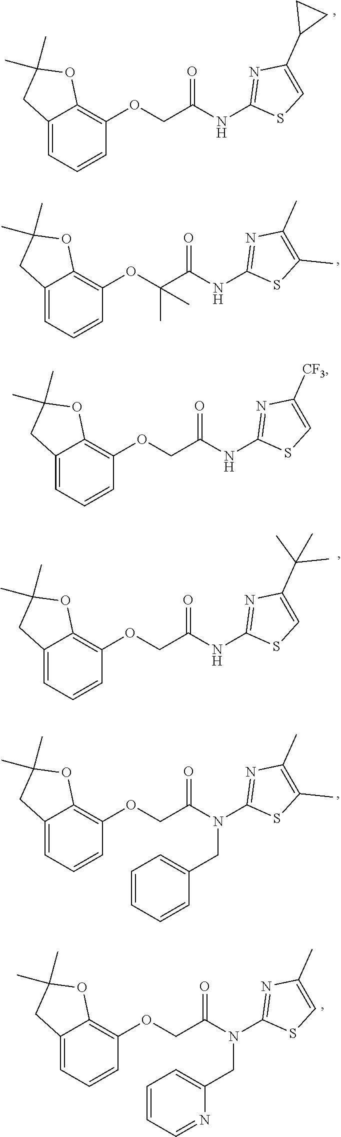 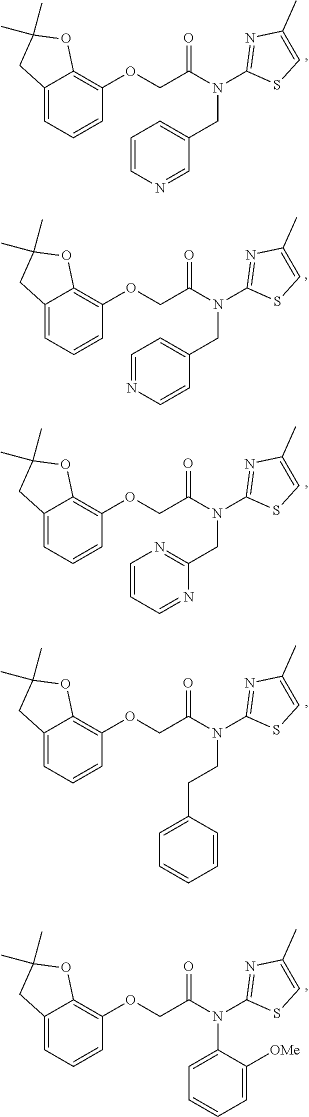 [SMILES: Cc1csc(N(Cc2cccnc2)C(=O)COc2cccc3c2OC(C)(C)C3)n1 ; Cc1csc(N(Cc2ccncc2)C(=O)COc2cccc3c2OC(C)(C)C3)n1 ; Cc1csc(N(Cc2ncccn2)C(=O)COc2cccc3c2OC(C)(C)C3)n1 ; Cc1csc(N(CCc2ccccc2)C(=O)COc2cccc3c2OC(C)(C)C3)n1 ; COc1ccccc1N(C(=O)COc1cccc2c1OC(C)(C)C2)c1nc(C)cs1] 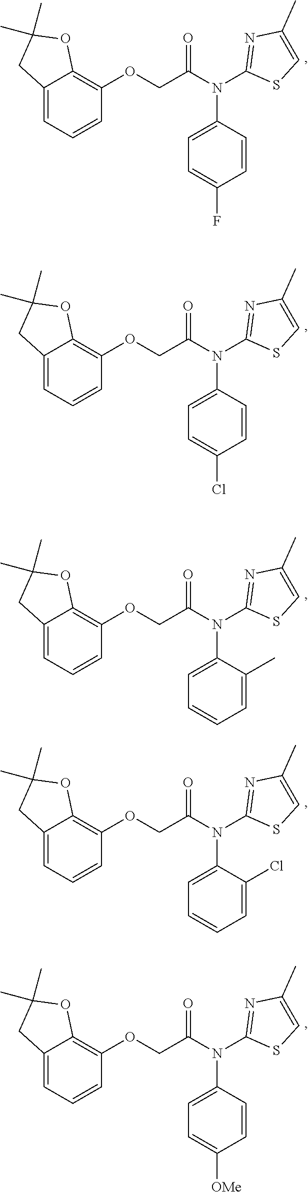 [SMILES: Cc1csc(N(C(=O)COc2cccc3c2OC(C)(C)C3)c2ccc(F)cc2)n1 ; Cc1csc(N(C(=O)COc2cccc3c2OC(C)(C)C3)c2ccc(Cl)cc2)n1 ; Cc1csc(N(C(=O)COc2cccc3c2OC(C)(C)C3)c2ccccc2C)n1 ; Cc1csc(N(C(=O)COc2cccc3c2OC(C)(C)C3)c2ccccc2Cl)n1 ; COc1ccc(N(C(=O)COc2cccc3c2OC(C)(C)C3)c2nc(C)cs2)cc1] 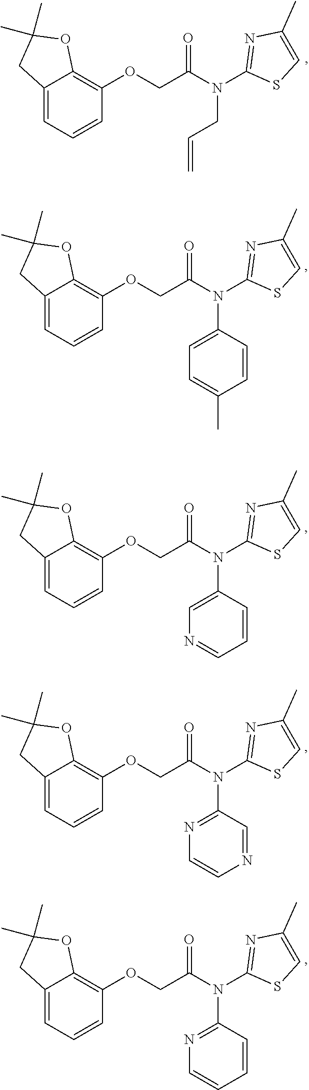 [SMILES: C=CCN(C(=O)COc1cccc2c1OC(C)(C)C2)c1nc(C)cs1 ; Cc1ccc(N(C(=O)COc2cccc3c2OC(C)(C)C3)c2nc(C)cs2)cc1 ; Cc1csc(N(C(=O)COc2cccc3c2OC(C)(C)C3)c2cccnc2)n1 ; Cc1csc(N(C(=O)COc2cccc3c2OC(C)(C)C3)c2cnccn2)n1 ; Cc1csc(N(C(=O)COc2cccc3c2OC(C)(C)C3)c2ccccn2)n1] 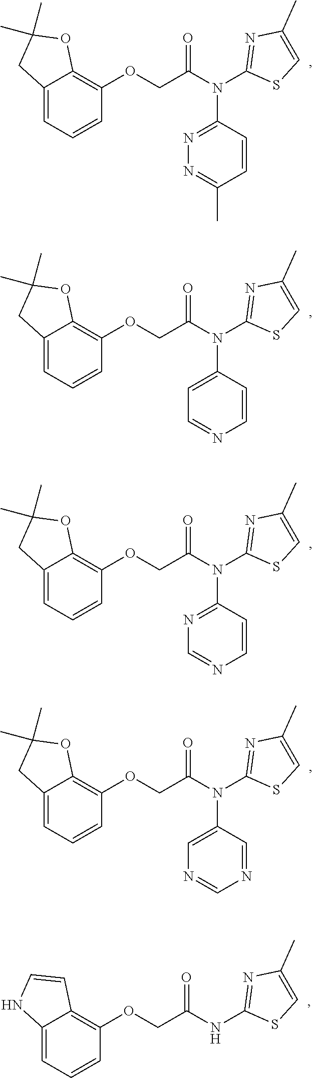 [SMILES: Cc1ccc(N(C(=O)COc2cccc3c2OC(C)(C)C3)c2nc(C)cs2)nn1 ; Cc1csc(N(C(=O)COc2cccc3c2OC(C)(C)C3)c2ccncc2)n1 ; Cc1csc(N(C(=O)COc2cccc3c2OC(C)(C)C3)c2ccncn2)n1 ; Cc1csc(N(C(=O)COc2cccc3c2OC(C)(C)C3)c2cncnc2)n1 ; Cc1csc(NC(=O)COc2cccc3[nH]ccc23)n1] 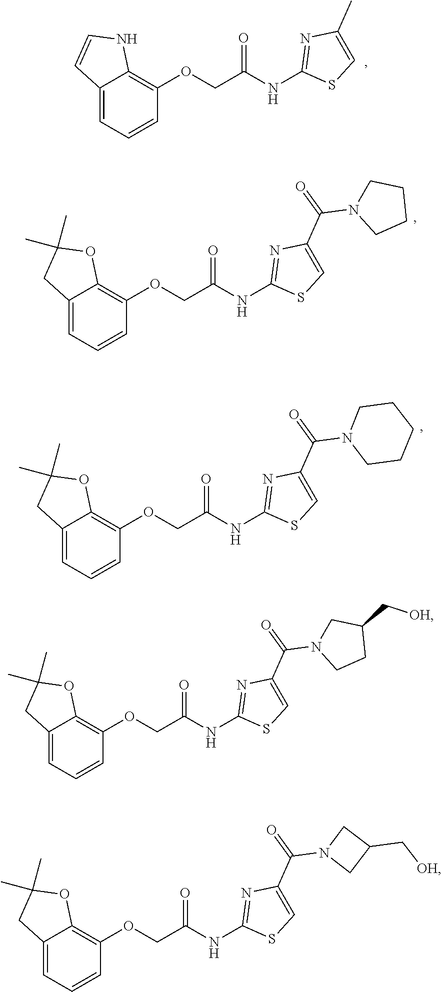 [SMILES: Cc1csc(NC(=O)COc2cccc3cc[nH]c23)n1 ; CC1(C)Cc2cccc(OCC(=O)Nc3nc(C(=O)N4CCCC4)cs3)c2O1 ; CC1(C)Cc2cccc(OCC(=O)Nc3nc(C(=O)N4CCCCC4)cs3)c2O1 ; CC1(C)Cc2cccc(OCC(=O)Nc3nc(C(=O)N4CC[C@H](CO)C4)cs3)c2O1 ; CC1(C)Cc2cccc(OCC(=O)Nc3nc(C(=O)N4CC(CO)C4)cs3)c2O1] 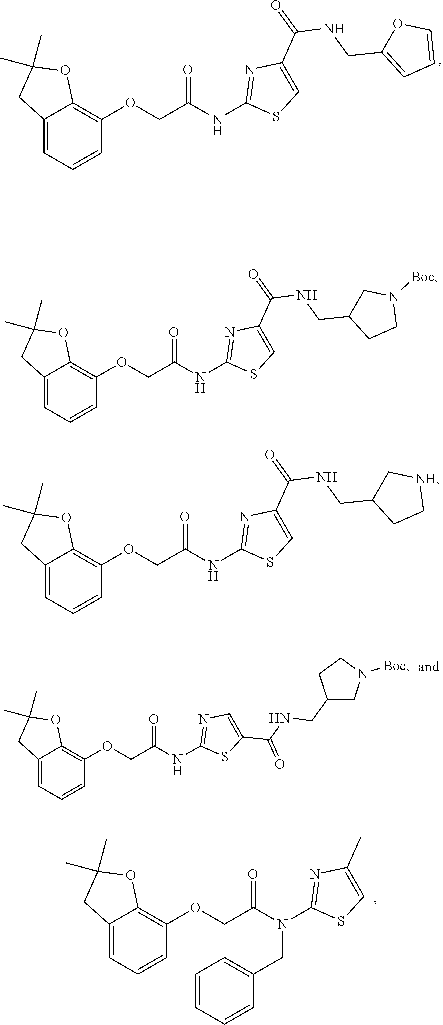 [SMILES: CC1(C)Cc2cccc(OCC(=O)Nc3nc(C(=O)NCc4ccco4)cs3)c2O1 ; CC(C)(C)OC(=O)N1CCC(CNC(=O)c2csc(NC(=O)COc3cccc4c3OC(C)(C)C4)n2)C1 ; CC1(C)Cc2cccc(OCC(=O)Nc3nc(C(=O)NCC4CCNC4)cs3)c2O1 ; CC(C)(C)OC(=O)N1CCC(CNC(=O)c2cnc(NC(=O)COc3cccc4c3OC(C)(C)C4)s2)C1 ; Cc1csc(N(Cc2ccccc2)C(=O)COc2cccc3c2OC(C)(C)C3)n1]
or a pharmaceutically acceptable salt thereof.

82. A compound, or a pharmaceutically acceptable salt thereof, of Formula V: 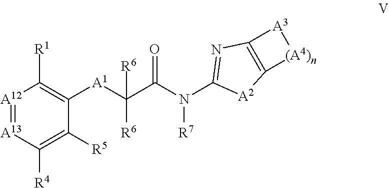
wherein:
R¹ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR⁴⁸, -carbocyclyl optionally substituted with 1-4 R⁴⁹, and —CN;
R² is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR⁴⁸, -carbocyclyl optionally substituted with 1-4 R⁴⁹, and —CN;
R³ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR⁴⁸, -carbocyclyl optionally substituted with 1-4 R⁴⁹, and —CN;
R⁴ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR⁴⁸, -carbocyclyl optionally substituted with 1-4 R⁴⁹, and —CN;
R⁵ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR⁴⁸, -carbocyclyl optionally substituted with 1-4 R⁴⁹, and —CN;
with the proviso that when A¹² is —C(R²)— and A¹³ is —C(R³)—, then R¹, R², R³, R⁴, and R⁵ are not all H;
with the proviso that when A¹² is N, then R¹, R³, R⁴, and R⁵ are not all H;
each R⁶ is independently selected from the group consisting of H, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
R⁷ is selected from the group consisting of unsubstituted —(C₁₋₉ alkyl), unsubstituted —(C₃₋₉ alkenyl), unsubstituted —(C₃₋₉ alkynyl), unsubstituted —(C₁₋₉ haloalkyl), —(C₁₋₄ alkylene)C(═O)OR¹¹, —(C₁₋₄ alkylene)C(═O)N(R¹²)₂, —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein;
each R¹⁰ is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
R¹¹ is selected from the group consisting of H and unsubstituted —(C₁₋₉ alkyl);
each R¹² is independently selected from the group consisting of H and substituted or unsubstituted —(C₁₋₉ alkyl);
each R¹³ is independently selected from the group consisting of halide, unsubstituted —(C₁₋₉ alkyl), unsubstituted —(C₁₋₉ haloalkyl), and —OR¹⁰;
each R¹⁴ is independently selected from the group consisting of unsubstituted —(C₁₋₉ alkyl), unsubstituted —(C₁₋₉ haloalkyl), and —OR¹⁰;
each R¹⁵ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁶ is independently selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
each R⁴⁸ is independently selected from the group consisting of unsubstituted —(C₁₋₉ alkyl), unsubstituted —(C₃₋₉ alkenyl), unsubstituted —(C₃₋₉ alkynyl), and unsubstituted —(C₁₋₉ haloalkyl);
each R⁴⁹ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
A¹ is selected from the group consisting of —C(R¹⁵)₂—, O, and S;
A² is selected from the group consisting of O and S;
A³ is selected from the group consisting of —C(R¹⁶)₂—, O and S;
each A⁴ is independently selected from the group consisting of —C(R¹⁶)₂—, O and S;
with the proviso that no more than two of A³ and/or A⁴ are O or S and that two O or S are never adjacent;
A¹² is selected from the group consisting of —C(R²)— and N;
A¹³ is selected from the group consisting of —C(R³)— and N;
n is 1 to 4;
p is 0 to 1; and
with the proviso that Formula V is not a structure selected from the group consisting of:

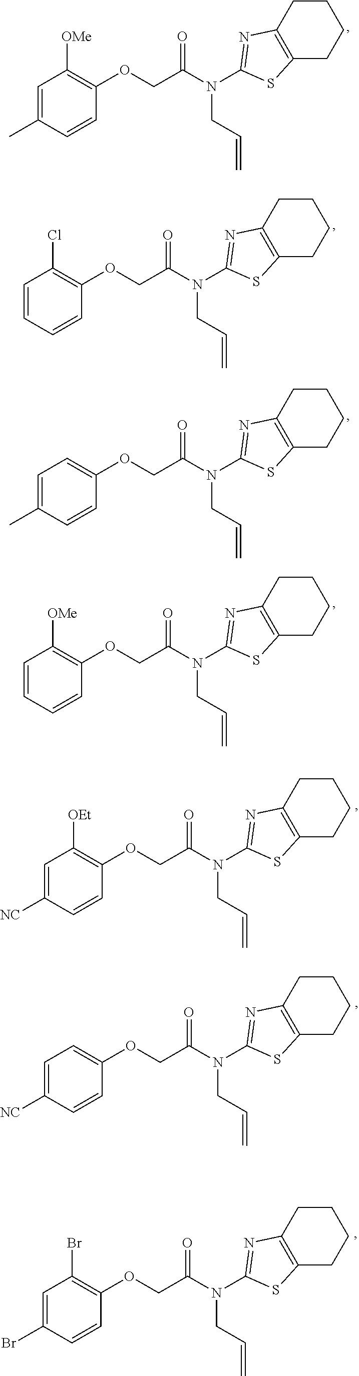

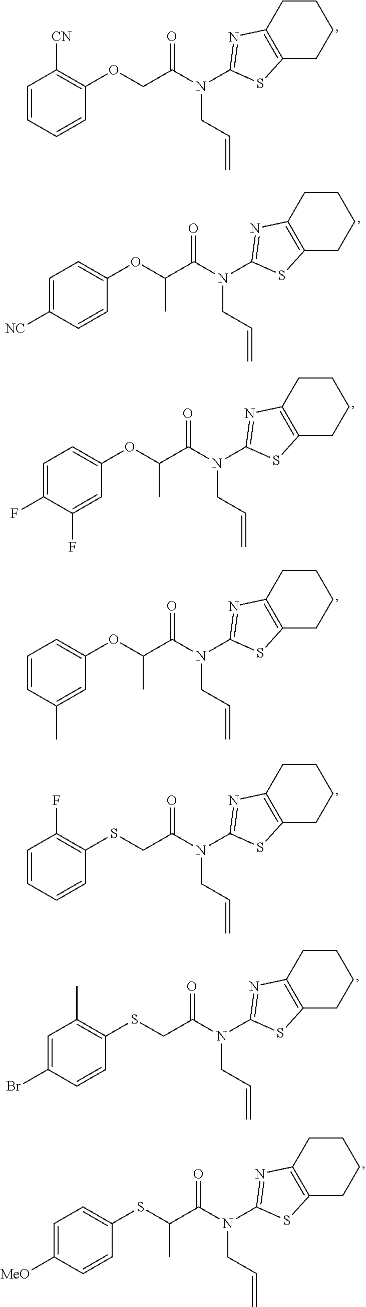

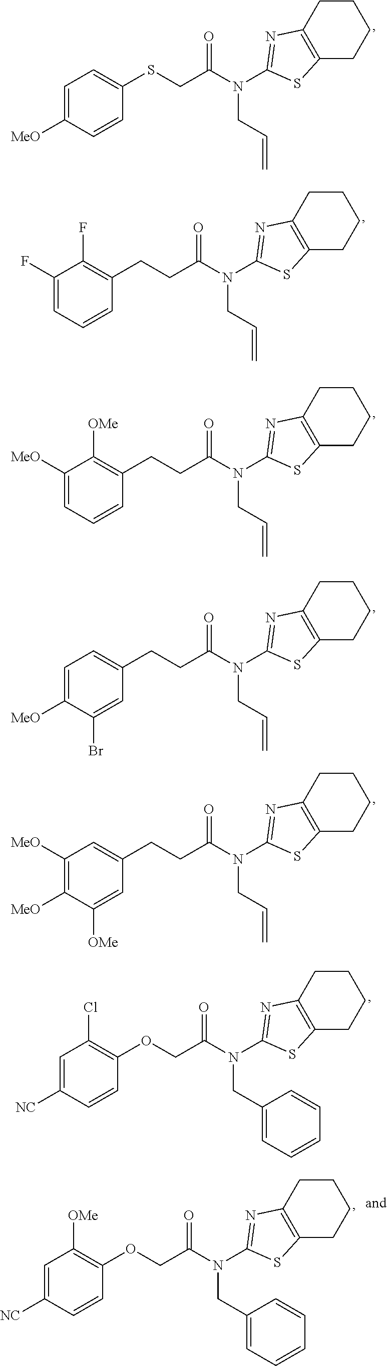

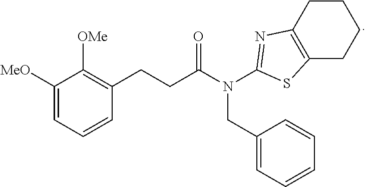 [SMILES: COc1cccc(CCC(=O)N(Cc2ccccc2)c2nc3c(s2)CCCC3)c1OC]

83. A compound, or a pharmaceutically acceptable salt thereof, of Formula V: 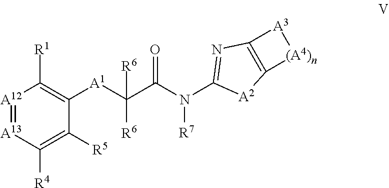
wherein:
R¹ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR⁴⁸, -carbocyclyl optionally substituted with 1-4 R⁴⁹, and —CN;
R² is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR⁴⁸, -carbocyclyl optionally substituted with 1-4 R⁴⁹, and —CN;
R³ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR⁴⁸, -carbocyclyl optionally substituted with 1-4 R⁴⁹, and —CN;
R⁴ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR⁴⁸, -carbocyclyl optionally substituted with 1-4 R⁴⁹, and —CN;
R⁵ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR⁴⁸, -carbocyclyl optionally substituted with 1-4 R⁴⁹, and —CN;
with the proviso that when A¹² is —C(R²)— and A¹³ is —C(R³)—, then R¹, R², R³, R⁴, and R⁵ are not all H;
with the proviso that when A¹² is N, then R¹, R³, R⁴, and R⁵ are not all H;
each R⁶ is independently selected from the group consisting of H, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
R⁷ is selected from the group consisting of unsubstituted —(C₁₋₉ alkyl), unsubstituted —(C₁₋₉ haloalkyl), —(C₁₋₄ alkylene)C(═O)OR¹¹, —(C₁₋₄ alkylene)C(═O)N(R¹²)₂, —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein;
with the proviso that when R⁷ is —(CH₂)aryl, then the aryl is not unsubstituted or substituted with halide or methyl;
each R¹⁰ is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
R¹¹ is selected from the group consisting of H and unsubstituted —(C₁₋₉ alkyl);
each R¹² is independently selected from the group consisting of H and substituted or unsubstituted —(C₁₋₉ alkyl);
each R¹³ is independently selected from the group consisting of halide, unsubstituted —(C₁₋₉ alkyl), unsubstituted —(C₁₋₉ haloalkyl), and —OR¹⁰;
each R¹⁴ is independently selected from the group consisting of unsubstituted —(C₁₋₉ alkyl), unsubstituted —(C₁₋₉ haloalkyl), and —OR¹⁰;
each R¹⁵ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁶ is independently selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
each R⁴⁸ is independently selected from the group consisting of unsubstituted —(C₁₋₉ alkyl), unsubstituted —(C₃₋₉ alkenyl), unsubstituted —(C₃₋₉ alkynyl), and unsubstituted —(C₁₋₉ haloalkyl);
each R⁴⁹ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
A¹ is selected from the group consisting of —C(R¹⁵)₂—, O, and S;
A² is selected from the group consisting of O and S;
A³ is selected from the group consisting of —C(R¹⁶)₂—, O and S;
each A⁴ is independently selected from the group consisting of —C(R¹⁶)₂—, O and S;
with the proviso that no more than two of A³ and/or A⁴ are O or S and that two O or S are never adjacent;
A¹² is selected from the group consisting of —C(R²)— and N;
A¹³ is selected from the group consisting of —C(R³)— and N;
n is 1, 2, 3, or 4; and
p is 0 or 1.

84. The compound of any one of claims 82-83, wherein each R⁶ is H.

85. The compound of any one of claims 82-84, wherein R¹ is selected from the group consisting of H, halide, unsubstituted —(C₁₋₃ alkyl), unsubstituted —(C₁₋₃ haloalkyl), —O(C₁₋₂ alkyl), and —CN;

86. The compound of any one of claims 82-85, wherein R¹ is selected from the group consisting of H, halide, methyl, —CF₃, —OMe, and —CN;

87. The compound of any one of claims 82-86, wherein R² is selected from the group consisting of H, halide, unsubstituted —(C₁₋₃ alkyl), unsubstituted —(C₁₋₃ haloalkyl), —O(C₁₋₂ alkyl), unsubstituted 3-4-membered carbocyclyl, and —CN;

88. The compound of any one of claims 82-87, wherein R² is selected from the group consisting of H, halide, methyl, —CF₃, —OMe, cyclopropyl, and —CN;

89. The compound of any one of claims 82-88, wherein R³ is selected from the group consisting of H, halide, unsubstituted —(C₁₋₂ alkyl), unsubstituted —(C₁₋₂ haloalkyl), —O(C₁₋₂ alkyl), and —CN;

90. The compound of any one of claims 82-89, wherein R³ is selected from the group consisting of H, halide, methyl, —CF₃, —OMe, and —CN;

91. The compound of any one of claims 82-90, wherein R⁴ is selected from the group consisting of H, halide, unsubstituted —(C₁₋₂ alkyl), unsubstituted —(C₁₋₂ haloalkyl), —O(C₁₋₂ alkyl), and —CN;

92. The compound of any one of claims 82-91, wherein R⁴ is selected from the group consisting of H, halide, methyl, —CF₃, —OMe, and —CN;

93. The compound of any one of claims 82-92, wherein R⁵ is selected from the group consisting of H, halide, unsubstituted —(C₁₋₂ alkyl), unsubstituted —(C₁₋₂ haloalkyl), —O(C₁₋₂ alkyl), and —CN;

94. The compound of any one of claims 82-93, wherein R⁵ is selected from the group consisting of H, halide, methyl, —CF₃, —OMe, and —CN;

95. The compound of any one of claims 82-94, wherein A² is S.

96. The compound of any one of claims 82-95, wherein A³ and all A⁴ are —C(R¹⁶)₂—.

97. The compound of any one of claims 82-96, wherein R¹⁶ is H.

98. The compound of any one of claims 82-97, wherein n is 2.

99. The compound of any one of claims 82-98, wherein n is 3.

100. The compound of any one of claims 82-99, wherein n is 4.

101. The compound of any of claims 82-100, wherein the compound of Formula V is selected from the group consisting of: 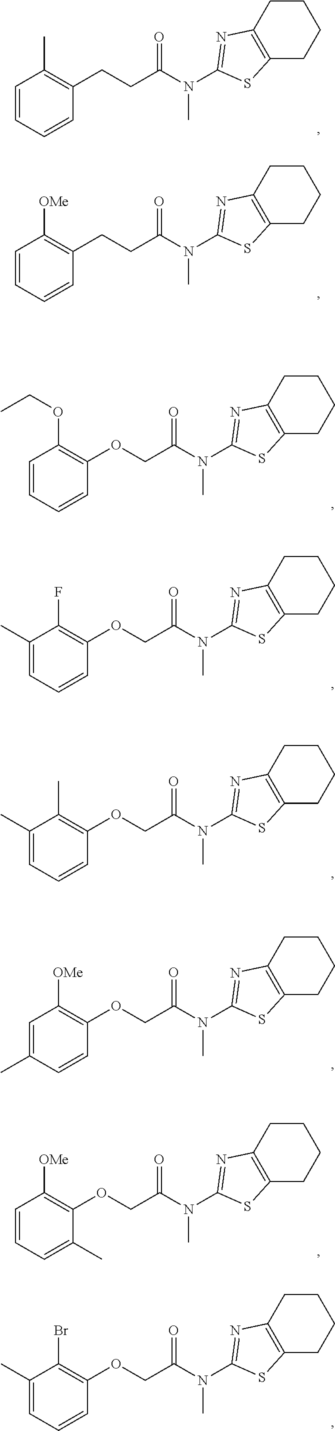
or a pharmaceutically acceptable salt thereof.

102. A compound, or a pharmaceutically acceptable salt thereof, of Formula VI: 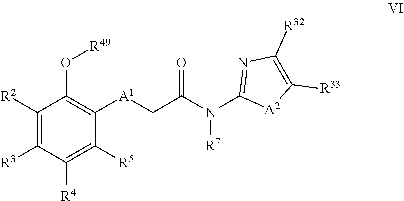
wherein:
R² is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R³ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R⁴ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R⁵ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R⁷ is selected from the group consisting of H, unsubstituted —(C₁₋₉ alkyl), unsubstituted —(C₃₋₉ alkenyl), unsubstituted —(C₃₋₉ alkynyl), unsubstituted —(C₁₋₉ haloalkyl), —(C₁₋₄ alkylene)C(═O)OR¹¹, —(C₁₋₄ alkylene)C(═O)N(R¹²)₂, —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein;
each R¹⁰ is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
R¹¹ is selected from the group consisting of H and unsubstituted —(C₁₋₉ alkyl);
each R¹² is independently selected from the group consisting of H and substituted or unsubstituted —(C₁₋₉ alkyl);
each R¹³ is independently selected from the group consisting of halide, unsubstituted —(C₁₋₉ alkyl), unsubstituted —(C₁₋₉ haloalkyl), and —OR¹⁰;
each R¹⁴ is independently selected from the group consisting of halide, unsubstituted —(C₁₋₉ alkyl), unsubstituted —(C₁₋₉ haloalkyl), and —OR¹⁰;
each R¹⁵ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
R³² is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ fluoroalkyl), and —CN;
R³³ is selected from the group consisting of H, -unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R⁴⁹ is selected from the group consisting of unsubstituted —(C₃₋₉ alkyl), unsubstituted —(C₃₋₉ alkenyl), unsubstituted —(C₃₋₉ alkynyl), unsubstituted —(C₃₋₉ haloalkyl), —(C₁₋₄ alkylene)ₚheteroaryl optionally substituted with 1-4 R⁵⁰, and —(C₁₋₄ alkylene)ₚaryl optionally substituted with 1-5 R⁵¹, and —(C₁₋₄ alkylene)ₚcarbocyclyl optionally substituted with 1-10 R⁵²; wherein each —(C₁₋₄ alkylene) is, independently, optionally substituted with one or more substituents as defined herein;
each R⁵⁰ is independently selected from the group consisting of halide, unsubstituted —(C₁₋₉ alkyl), unsubstituted —(C₁₋₉ haloalkyl), and —OR¹⁰;
each R⁵¹ is independently selected from the group consisting of halide, unsubstituted —(C₁₋₉ alkyl), unsubstituted —(C₁₋₉ haloalkyl), and —OR¹⁰;
each R⁵² is independently selected from the group consisting of halide, unsubstituted —(C₁₋₉ alkyl), and unsubstituted —(C₁₋₉ haloalkyl);
A¹ is selected from the group consisting of —C(R¹⁵)₂—, O, and S;
A² is selected from the group consisting of O and S;
each p independently is 0 or 1; and
with the proviso that Formula VI is not:

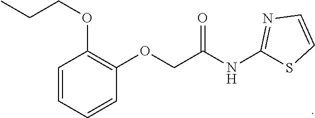 [SMILES: CCCOc1ccccc1OCC(=O)Nc1nccs1]

103. A compound, or a pharmaceutically acceptable salt thereof, of Formula VI: 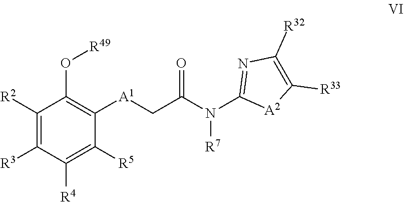
wherein:
R² is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R³ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R⁴ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R⁵ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R⁷ is selected from the group consisting of H, unsubstituted —(C₁₋₉ alkyl), unsubstituted —(C₃₋₉ alkenyl), unsubstituted —(C₃₋₉ alkynyl), unsubstituted —(C₁₋₉ haloalkyl), —(C₁₋₄ alkylene)C(═O)OR¹¹, —(C₁₋₄ alkylene)C(═O)N(R¹²)₂, —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein;
each R¹⁰ is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
R¹¹ is selected from the group consisting of H and unsubstituted —(C₁₋₉ alkyl);
each R¹² is independently selected from the group consisting of H and substituted or unsubstituted —(C₁₋₉ alkyl);
each R¹³ is independently selected from the group consisting of halide, unsubstituted —(C₁₋₉ alkyl), unsubstituted —(C₁₋₉ haloalkyl), and —OR¹⁰;
each R¹⁴ is independently selected from the group consisting of halide, unsubstituted —(C₁₋₉ alkyl), unsubstituted —(C₁₋₉ haloalkyl), and —OR¹⁰;
each R¹⁵ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
R³² is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ fluoroalkyl), and —CN;
R³³ is selected from the group consisting of H, -unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R⁴⁹ is selected from the group consisting of unsubstituted —(C₃₋₉ alkyl), unsubstituted —(C₃₋₉ alkenyl), unsubstituted —(C₃₋₉ alkynyl), unsubstituted —(C₃₋₉ haloalkyl), —(C₁₋₄ alkylene)ₚheteroaryl optionally substituted with 1-4 R⁵⁰, and —(C₁₋₄ alkylene)ₚaryl optionally substituted with 1-5 R⁵¹, and —(C₁₋₄ alkylene)ₚcarbocyclyl optionally substituted with 1-10 R⁵²; wherein each —(C₁₋₄ alkylene) is, independently, optionally substituted with one or more substituents as defined herein;
with the proviso that when R⁷ is H, then R⁴⁹ is not —(C₃ alkyl);
each R⁵⁰ is independently selected from the group consisting of halide, unsubstituted —(C₁₋₉ alkyl), unsubstituted —(C₁₋₉ haloalkyl), and —OR¹⁰;
each R⁵¹ is independently selected from the group consisting of halide, unsubstituted —(C₁₋₉ alkyl), unsubstituted —(C₁₋₉ haloalkyl), and —OR¹⁰;
each R⁵² is selected from the group consisting of halide, unsubstituted —(C₁₋₉ alkyl), and unsubstituted —(C₁₋₉ haloalkyl);
A¹ is selected from the group consisting of —C(R¹⁵)₂—, O, and S;
A² is selected from the group consisting of O and S; and
each p independently is 0 or 1.

104. The compound of any one of claims 102-103, wherein R² is selected from the group consisting of H and halide.

105. The compound of any one of claims 102-104, wherein R⁴⁹ is selected from the group consisting of unsubstituted —(C₃₋₅ alkyl), unsubstituted —(C₃₋₅ alkenyl), unsubstituted —(C₃₋₅ alkynyl), unsubstituted —(C₃₋₅ haloalkyl), —(CH₂)heteroaryl optionally substituted with 1-4 R⁵⁰, and —(CH₂)aryl optionally substituted with 1-5 R⁵¹, and —(CH₂)carbocyclyl optionally substituted with 1-10 R⁵².

106. The compound of any one of claims 102-105, wherein R⁴⁹ is selected from the group consisting of unsubstituted —(C₃₋₅ alkyl), and —(CH₂)aryl optionally substituted with 1-5 R⁵¹.

107. The compound of any one of claims 102-106, wherein R⁵⁰, R⁵¹ and R⁵² are independently selected from the group consisting of F, Cl, Br, and I.

108. The compound of any one of claims 102-107, wherein A² is S.

109. The compound of any one of claims 102-108, wherein R³² is selected from the group consisting of H, halide, unsubstituted —(C₁₋₄ alkyl), unsubstituted —(C₁₋₃fluoroalkyl), and —CN.

110. The compound of any one of claims 102-109, wherein R³³ is selected from the group consisting of H, methyl, —CF₃, and —CN.

111. The compound of any of claims 102-110, wherein the compound of Formula VI is selected from the group consisting of: 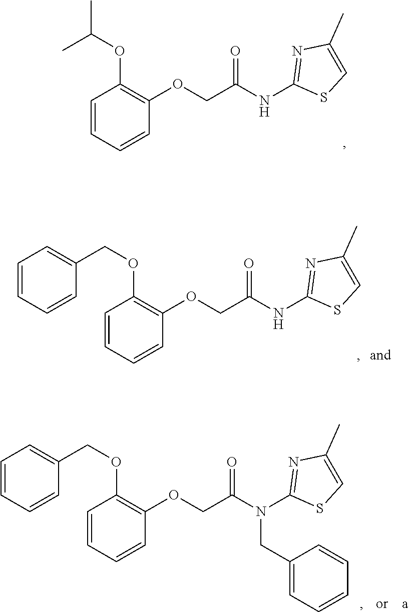 [SMILES: Cc1csc(NC(=O)COc2ccccc2OC(C)C)n1 ; Cc1csc(NC(=O)COc2ccccc2OCc2ccccc2)n1 ; Cc1csc(N(Cc2ccccc2)C(=O)COc2ccccc2OCc2ccccc2)n1]
pharmaceutically acceptable salt thereof.

112. The compound of any one of claims 1-111, wherein A¹ is O.

113. The compound of any one of claims 1-112, wherein A¹ is S.

114. The compound of any one of claims 1-113, wherein A¹ is —CH₂—.

115. The compound of any one of claims 1-114, wherein R³, R⁴, and R⁵ are independently selected from the group consisting of H and halide.

116. The compound of any one of claims 1-115, wherein R³, R⁴, and R⁵ are H.

117. The compound of any one of claims 1-116, wherein R⁷ is selected from the group consisting of H, unsubstituted —(C₁₋₃ alkyl), unsubstituted —(C₃₋₄ alkenyl), —(CH₂)C(═O)O(C₁₋₃ alkyl), -heteroaryl optionally substituted with 1-4 R¹³, —(CH₂)heteroaryl optionally substituted with 1-4 R¹³, -aryl optionally substituted with 1-5 R¹⁴, and —(CH₂)aryl optionally substituted with 1-5 R¹⁴.

118. The compound of any one of claims 1-117, wherein R⁷ is H.

119. The compound of any one of claims 1-118, wherein R⁷ is unsubstituted —(C₁₋₃ alkyl).

120. The compound of any one of claims 1-119, wherein R⁷ is -heteroaryl optionally substituted with 1-4 R¹³.

121. The compound of any one of claims 1-120, wherein R⁷ is —(CH₂)heteroaryl optionally substituted with 1-4 R¹³.

122. The compound of any one of claims 1-121, wherein R⁷ is -aryl optionally substituted with 1-5 R¹⁴.

123. The compound of any one of claims 1-122, wherein R⁷ is —(CH₂)aryl optionally substituted with 1-5 R¹⁴.

124. The compound of any one of claims 1-123, wherein R⁷ is selected from the group consisting of

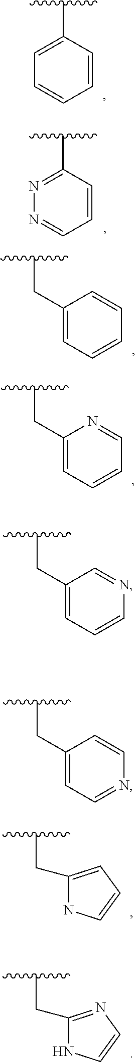

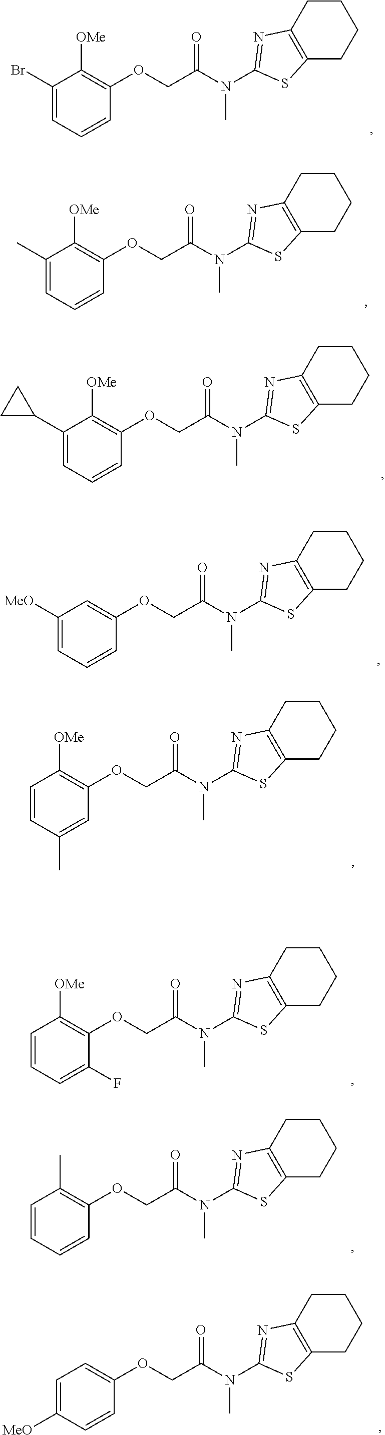

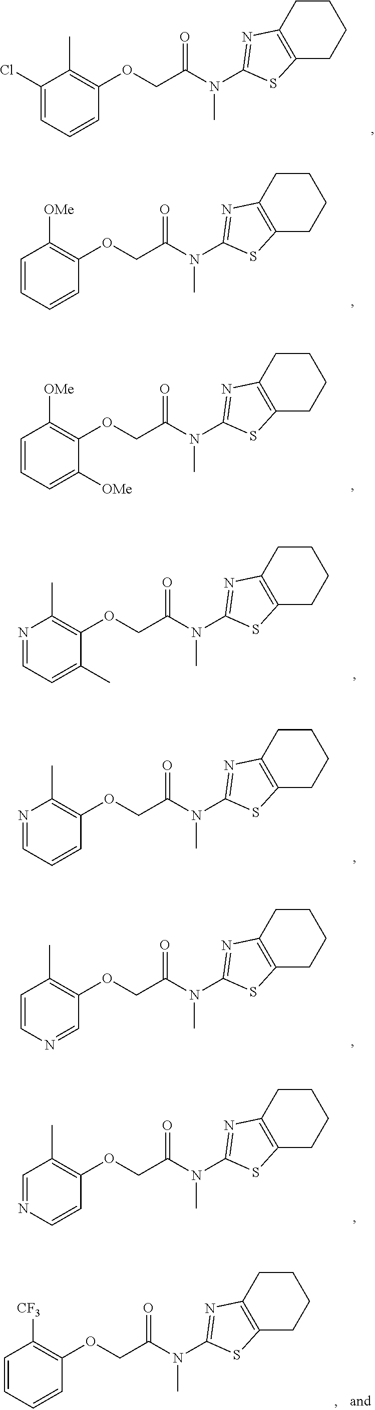

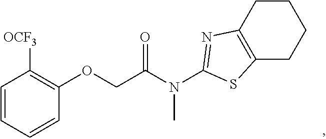 [SMILES: CN(C(=O)COc1ccccc1OC(F)(F)F)c1nc2c(s1)CCCC2]

125. A pharmaceutical composition comprising a therapeutically effective amount of a compound according to any of claims 1-124, or a pharmaceutically acceptable salt thereof, and a pharmaceutically acceptable excipient.

126. A method of treating a disorder or disease in a patient, wherein the disorder or disease is selected from the group consisting of: human immunodeficiency virus 1 (HIV-1), human immunodeficiency virus 2 (HIV-2) and simian immunodeficiency virus (SIV), the method comprising administering to the patient a therapeutically effective amount of a compound, or a pharmaceutically acceptable salt thereof, of Formula I: 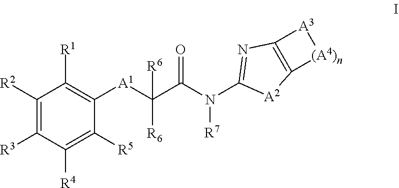
wherein:
R¹ and R² are taken together to form a ring which is selected from the group consisting of heteroaryl optionally substituted with 1-4 R⁸ and heterocyclyl optionally substituted with 1-4 R⁹;
R³, R⁴, and R⁵ are independently selected from the group consisting of H, halide, unsubstituted —(C₁₋₆ alkyl), unsubstituted —(C₁₋₄ haloalkyl), —OR¹⁰, and —CN;
each R⁶ is independently selected from the group consisting of H, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein;
R⁷ is selected from the group consisting of H, unsubstituted —(C₁₋₉ alkyl), unsubstituted —(C₃₋₉ alkenyl), unsubstituted —(C₃₋₉ alkynyl), unsubstituted —(C₁₋₉ haloalkyl), —(C₁₋₄ alkylene)C(═O)OR¹¹, —(C₁₋₄ alkylene)C(═O)N(R¹²)₂, —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein;
each R⁸ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR¹⁰, and —CN;
each R⁹ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
each R¹⁰ is independently selected from the group consisting of H, unsubstituted —(C₁₋₉ alkyl), unsubstituted —(C₃₋₉ alkenyl), unsubstituted —(C₃₋₉ alkynyl), and unsubstituted —(C₁₋₉ haloalkyl);
each R¹¹ is independently selected from the group consisting of H and unsubstituted —(C₁₋₉ alkyl);
each R¹² is independently selected from the group consisting of H and substituted or unsubstituted —(C₁₋₉ alkyl);
each R¹³ is independently selected from the group consisting of halide, unsubstituted —(C₁₋₉ alkyl), unsubstituted —(C₁₋₉ haloalkyl), and —OR¹⁰;
each R¹⁴ is independently selected from the group consisting of halide, unsubstituted —(C₁₋₉ alkyl), unsubstituted —(C₁₋₉ haloalkyl), and —OR¹⁰;
each R¹⁵ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁶ is independently selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
A¹ is selected from the group consisting of —C(R¹⁵)₂—, O, and S;
A² is selected from the group consisting of O and S;
A³ is selected from the group consisting of —C(R¹⁶)₂—, O and S;
each A⁴ is independently selected from the group consisting of —C(R¹⁶)₂—, O and S;
with the proviso that no more than two A³ and A⁴ are O or S and that two O or S are never adjacent;
n is 1, 2, 3, or 4;
each p independently is 0 or 1; and
with the proviso that Formula I is not a structure selected from the group consisting of:

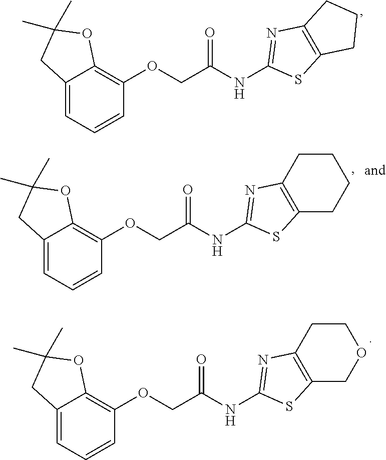

127. A method of treating a disorder or disease in a patient, wherein the disorder or disease is selected from the group consisting of: human immunodeficiency virus 1 (HIV-1), human immunodeficiency virus 2 (HIV-2) and simian immunodeficiency virus (SIV), the method comprising administering to the patient a therapeutically effective amount of a compound, or a pharmaceutically acceptable salt thereof, of Formula I: 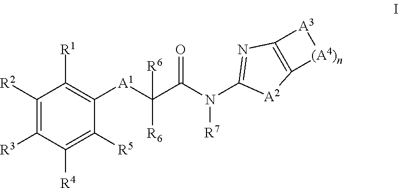
wherein:
R¹ and R² are taken together to form a ring which is selected from the group consisting of heteroaryl optionally substituted with 1-4 R⁸ and heterocyclyl optionally substituted with 1-4 R⁹;
R³, R⁴, and R⁵ are independently selected from the group consisting of H, halide, unsubstituted —(C₁₋₆ alkyl), unsubstituted —(C₁₋₄ haloalkyl), —OR¹⁰, and —CN;
each R⁶ is independently selected from the group consisting of H, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein;
R⁷ is selected from the group consisting of H, unsubstituted —(C₁₋₉ alkyl), unsubstituted —(C₃₋₉ alkenyl), unsubstituted —(C₃₋₉ alkynyl), unsubstituted —(C₁₋₉ haloalkyl), —(C₁₋₄ alkylene)C(═O)OR¹¹, —(C₁₋₄ alkylene)C(═O)N(R¹²)₂, —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein;
each R⁸ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR¹⁰, and —CN;
each R⁹ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
each R¹⁰ is independently selected from the group consisting of H, unsubstituted —(C₁₋₉ alkyl), unsubstituted —(C₃₋₉ alkenyl), unsubstituted —(C₃₋₉ alkynyl), and unsubstituted —(C₁₋₉ haloalkyl);
each R¹¹ is independently selected from the group consisting of H and unsubstituted —(C₁₋₉ alkyl);
each R¹² is independently selected from the group consisting of H and substituted or unsubstituted —(C₁₋₉ alkyl);
each R¹³ is independently selected from the group consisting of halide, unsubstituted —(C₁₋₉ alkyl), unsubstituted —(C₁₋₉ haloalkyl), and —OR¹⁰;
each R¹⁴ is independently selected from the group consisting of halide, unsubstituted —(C₁₋₉ alkyl), unsubstituted —(C₁₋₉ haloalkyl), and —OR¹⁰;
each R¹⁵ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁶ is independently selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
A¹ is selected from the group consisting of —C(R¹⁵)₂—, O, and S;
A² is selected from the group consisting of O and S;
A³ is selected from the group consisting of —C(R¹⁶)₂—, O and S;
each A⁴ is independently selected from the group consisting of —C(R¹⁶)₂—, O and S;
with the proviso that no more than two A³ and A⁴ are O or S and that two O or S are never adjacent;
n is 1, 2, 3, or 4; and
each p independently is 0 or 1.

128. A method of treating a disorder or disease in a patient, wherein the disorder or disease is selected from the group consisting of: human immunodeficiency virus 1 (HIV-1), human immunodeficiency virus 2 (HIV-2) and simian immunodeficiency virus (SIV), the method comprising administering to the patient a therapeutically effective amount of a compound, or a pharmaceutically acceptable salt thereof, of Formula II: 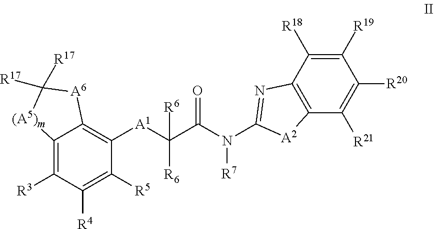
wherein:
R³, R⁴, and R⁵ are independently selected from the group consisting of H, halide, unsubstituted —(C₁₋₆ alkyl), unsubstituted —(C₁₋₄ haloalkyl), —OR¹⁰, and —CN;
each R⁶ is independently selected from the group consisting of H, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
R⁷ is selected from the group consisting of H, unsubstituted —(C₁₋₉ alkyl), unsubstituted —(C₃₋₉ alkenyl), unsubstituted —(C₃₋₉ alkynyl), unsubstituted —(C₁₋₉ haloalkyl), —(C₁₋₄ alkylene)C(═O)OR¹¹, —(C₁₋₄ alkylene)C(═O)N(R¹²)₂, —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein as defined herein;
each R¹⁰ is independently selected from the group consisting of H, unsubstituted —(C₁₋₉ alkyl), unsubstituted —(C₃₋₉ alkenyl), unsubstituted —(C₃₋₉ alkynyl), and unsubstituted —(C₁₋₉ haloalkyl);
R¹¹ is selected from the group consisting of H and unsubstituted —(C₁₋₉ alkyl);
each R¹² is independently selected from the group consisting of H and substituted or unsubstituted —(C₁₋₉ alkyl);
each R¹³ is independently selected from the group consisting of halide, unsubstituted —(C₁₋₉ alkyl), unsubstituted —(C₁₋₉ haloalkyl), and —OR¹⁰;
each R¹⁴ is independently selected from the group consisting of halide, unsubstituted —(C₁₋₉ alkyl), unsubstituted —(C₁₋₉ haloalkyl), and —OR¹⁰;
each R¹⁵ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁶ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁷ is independently selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R¹⁸ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR¹⁰, —(C₁₋₄ alkylene)ₚheteroaryl optionally substituted with 1-4 R²², —(C₁₋₄ alkylene)ₚaryl optionally substituted with 1-5 R²³, and —CN; wherein each —(C₁₋₄ alkylene) is, independently, optionally substituted with one or more substituents as defined herein;
R¹⁹ is selected from the group consisting of H, halide, unsubstituted —(C₁₋₉ alkyl), and unsubstituted —(C₁₋₆ haloalkyl);
R²⁰ is selected from the group consisting of H, halide, unsubstituted —(C₁₋₉ alkyl), and unsubstituted —(C₁₋₆ haloalkyl);
R²¹ is selected from the group consisting of H, halide, unsubstituted —(C₁₋₉ alkyl), and unsubstituted —(C₁₋₆ haloalkyl);
each R²² is independently selected from the group consisting of halide, unsubstituted —(C₁₋₉ alkyl), unsubstituted —(C₁₋₉ haloalkyl), and —CN;
each R²³ is independently selected from the group consisting of halide, unsubstituted —(C₁₋₉ alkyl), unsubstituted —(C₁₋₉ haloalkyl), and —CN;
A¹ is selected from the group consisting of —C(R¹⁵)₂—, O, and S;
A² is selected from the group consisting of O and S;
each A⁵ is independently selected from the group consisting of —C(R¹⁶)₂—, —N(R¹⁰)—, O, and S;
with the proviso that two A⁵ are not both N, O or S;
A⁶ is selected from the group consisting of —C(R¹⁶)₂—, —N(R¹⁰)—, O, and S;
with the proviso that A⁵ and A⁶ cannot both be C(R¹⁶)₂;
m is 1 or 2;
each p independently is 0 or 1; and
with the proviso that Formula II is not a structure selected from the group consisting of:

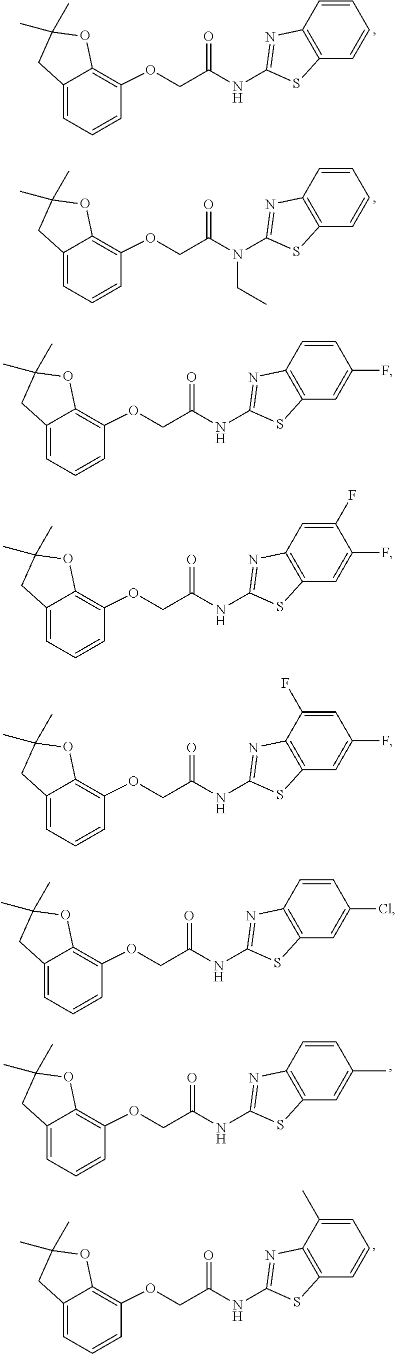

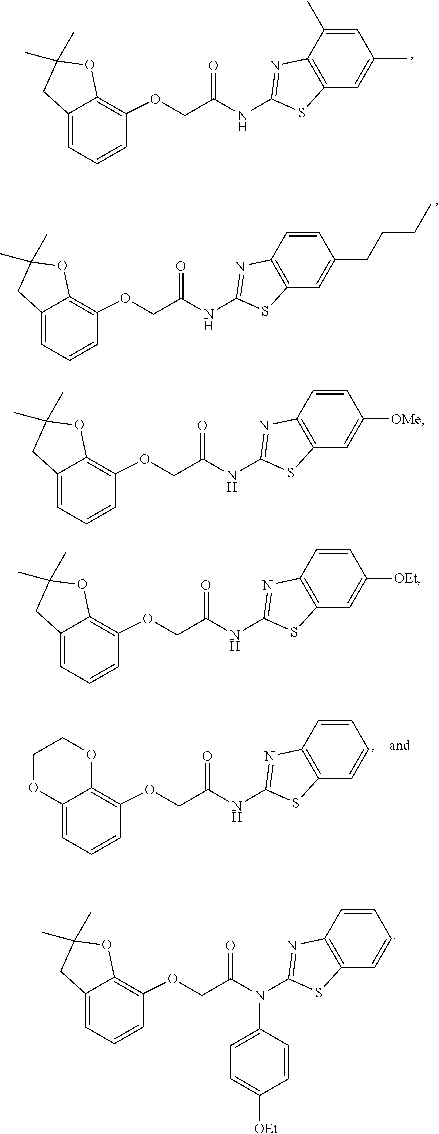

129. A method of treating a disorder or disease in a patient, wherein the disorder or disease is selected from the group consisting of: human immunodeficiency virus 1 (HIV-1), human immunodeficiency virus 2 (HIV-2) and simian immunodeficiency virus (SIV), the method comprising administering to the patient a therapeutically effective amount of a compound, or a pharmaceutically acceptable salt thereof, of Formula II: 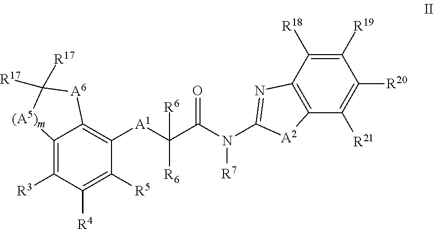
wherein:
R³, R⁴, and R⁵ are independently selected from the group consisting of H, halide, unsubstituted —(C₁₋₆ alkyl), unsubstituted —(C₁₋₄ haloalkyl), —OR¹⁰, and —CN;
each R⁶ is independently selected from the group consisting of H, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
R⁷ is selected from the group consisting of H, unsubstituted —(C₁₋₉ alkyl), unsubstituted —(C₃₋₉ alkenyl), unsubstituted —(C₃₋₉ alkynyl), unsubstituted —(C₁₋₉ haloalkyl), —(C₁₋₄ alkylene)C(═O)OR¹¹, —(C₁₋₄ alkylene)C(═O)N(R¹²)₂, —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein;
each R¹⁰ is independently selected from the group consisting of H, unsubstituted —(C₁₋₉ alkyl), unsubstituted —(C₃₋₉ alkenyl), unsubstituted —(C₃₋₉ alkynyl), and unsubstituted —(C₁₋₉ haloalkyl);
R¹¹ is selected from the group consisting of H and unsubstituted —(C₁₋₉ alkyl);
each R¹² is independently selected from the group consisting of H and substituted or unsubstituted —(C₁₋₉ alkyl);
each R¹³ is independently selected from the group consisting of halide, unsubstituted —(C₁₋₉ alkyl), unsubstituted —(C₁₋₉ haloalkyl), and —OR¹⁰;
each R¹⁴ is independently selected from the group consisting of halide, unsubstituted —(C₁₋₉ alkyl), unsubstituted —(C₁₋₉ haloalkyl), and —OR¹⁰;
each R¹⁵ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁶ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁷ is independently selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R¹⁸ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR¹⁰, —(C₁₋₄ alkylene)ₚheteroaryl optionally substituted with 1-4 R²², —(C₁₋₄ alkylene)ₚaryl optionally substituted with 1-5 R²³, and —CN; wherein each —(C₁₋₄ alkylene) is, independently, optionally substituted with one or more substituents as defined herein;
R¹⁹ is selected from the group consisting of H, halide, unsubstituted —(C₁₋₉ alkyl), and unsubstituted —(C₁₋₆ haloalkyl);
R²⁰ is selected from the group consisting of H, halide, unsubstituted —(C₁₋₉ alkyl), and unsubstituted —(C₁₋₆ haloalkyl);
R²¹ is selected from the group consisting of H, halide, unsubstituted —(C₁₋₉ alkyl), and unsubstituted —(C₁₋₆ haloalkyl);
each R²² is independently selected from the group consisting of halide, unsubstituted —(C₁₋₉ alkyl), unsubstituted —(C₁₋₉ haloalkyl), and —CN;
each R²³ is independently selected from the group consisting of halide, unsubstituted —(C₁₋₉ alkyl), unsubstituted —(C₁₋₉ haloalkyl), and —CN;
A¹ is selected from the group consisting of —C(R¹⁵)₂—, O, and S;
A² is selected from the group consisting of O and S;
each A⁵ is independently selected from the group consisting of —C(R¹⁶)₂—, —N(R¹⁰)—, O, and S;
with the proviso that two A⁵ are not both N, O or S;
A⁶ is selected from the group consisting of —C(R¹⁶)₂—, —N(R¹⁰)—, O, and S;
with the proviso that A⁵ and A⁶ cannot both be C(R¹⁶)₂;
m is 1 or 2; and
each p independently is 0 or 1.

130. A method of treating a disorder or disease in a patient, wherein the disorder or disease is selected from the group consisting of: human immunodeficiency virus 1 (HIV-1), human immunodeficiency virus 2 (HIV-2) and simian immunodeficiency virus (SIV), the method comprising administering to the patient a therapeutically effective amount of a compound, or a pharmaceutically acceptable salt thereof, of Formula III: 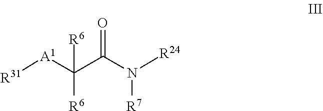
wherein:
R³, R⁴, and R⁵ are independently selected from the group consisting of H, halide, unsubstituted —(C₁₋₆ alkyl), unsubstituted —(C₁₋₄ haloalkyl), —OR¹⁰, and —CN;
each R⁶ is independently selected from the group consisting of H, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein;
R⁷ is selected from the group consisting of H, unsubstituted —(C₁₋₉ alkyl), unsubstituted —(C₃₋₉ alkenyl), unsubstituted —(C₃₋₉ alkynyl), unsubstituted —(C₁₋₉ haloalkyl), —(C₁₋₄ alkylene)C(═O)OR¹¹, —(C₁₋₄ alkylene)C(═O)N(R¹²)₂, —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein;
each R¹⁰ is independently selected from the group consisting of H, unsubstituted —(C₁₋₉ alkyl), unsubstituted —(C₃₋₉ alkenyl), unsubstituted —(C₃₋₉ alkynyl), and unsubstituted —(C₁₋₉ haloalkyl);
R¹¹ is selected from the group consisting of H and unsubstituted —(C₁₋₉ alkyl);
each R¹² is independently selected from the group consisting of H and substituted or unsubstituted —(C₁₋₉ alkyl);
each R¹³ is independently selected from the group consisting of halide, unsubstituted —(C₁₋₉ alkyl), unsubstituted —(C₁₋₉ haloalkyl), and —OR¹⁰;
each R¹⁴ is independently selected from the group consisting of halide, unsubstituted —(C₁₋₉ alkyl), unsubstituted —(C₁₋₉ haloalkyl), and —OR¹⁰;
each R¹⁵ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁶ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁷ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R²⁴ is selected from the group consisting of a 6-membered heteroaryl optionally substituted with 1-4 R²⁵, 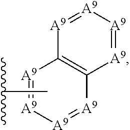
wherein 1-4 A⁹ are N; and 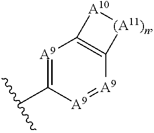
wherein 1-2 A⁹ are N;
each R²⁵ is independently selected from the group consisting of halide, —CN, —OR²⁸, unsubstituted —(C₁₋₆ alkyl), unsubstituted —(C₂₋₆ alkenyl), unsubstituted —(C₂₋₆ alkynyl), unsubstituted —(C₁₋₆ haloalkyl), and carbocyclyl optionally substituted with 1-10 R²⁹;
each R²⁶ is independently selected from the group consisting of H, halide, —CN, —OR²⁸, unsubstituted —(C₁₋₆ alkyl), unsubstituted —(C₂₋₆ alkenyl), unsubstituted —(C₂₋₆ alkynyl), unsubstituted —(C₁₋₆ haloalkyl), and carbocyclyl optionally substituted with 1-10 R²⁹;
each R²⁷ is independently selected from the group consisting of H, halide, —CN, —OR²⁸, unsubstituted —(C₁₋₆ alkyl), unsubstituted —(C₂₋₆ alkenyl), unsubstituted —(C₂₋₆ alkynyl), unsubstituted —(C₁₋₆ haloalkyl), and carbocyclyl optionally substituted with 1-10 R²⁹;
each R²⁸ is independently selected from the group consisting of unsubstituted —(C₁₋₉ alkyl), unsubstituted —(C₃₋₉ alkenyl), unsubstituted —(C₃₋₉ alkynyl), and unsubstituted —(C₁₋₉ haloalkyl);
each R²⁹ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
R³⁰ is selected from the group consisting of H and unsubstituted —(C₁₋₉ alkyl);
R³¹ is selected from the group consisting of 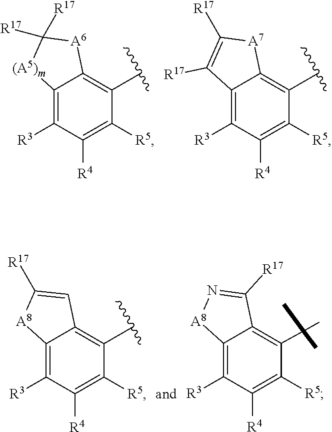
A¹ is selected from the group consisting of —C(R¹⁵)₂—, O, and S;
each A⁵ is independently selected from the group consisting of —C(R¹⁶)₂—, —N(R¹⁰)—, O, and S;
with the proviso that two A⁵ are not both N, O or S;
A⁶ is selected from the group consisting of —C(R¹⁶)₂—, —N(R¹⁰)—, O, and S;
with the proviso that A⁵ and A⁶ cannot both be C(R¹⁶)₂;
A⁷ is selected from the group consisting of —N(R³⁰)—, O and S;
A⁸ is selected from the group consisting of —NH—, O and S;
each A⁹ is independently selected from the group consisting of N and —C(R²⁶)—;
A¹⁰ is selected from the group consisting of —C(R²⁷)₂—, O, and S;
each A¹¹ is independently selected from the group consisting of —C(R²⁷)₂—, O, and S;
with the proviso that no more than two A¹⁰ or A¹¹ are O or S and that two O or S are never adjacent;
n is 1, 2, 3, or 4;
m is 1 or 2;
each p independently is 0 or 1; and
with the proviso that Formula III is not a structure selected from the group consisting of:

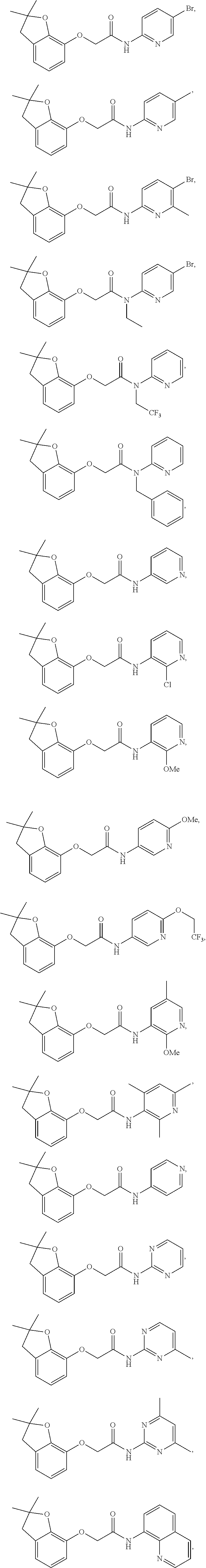

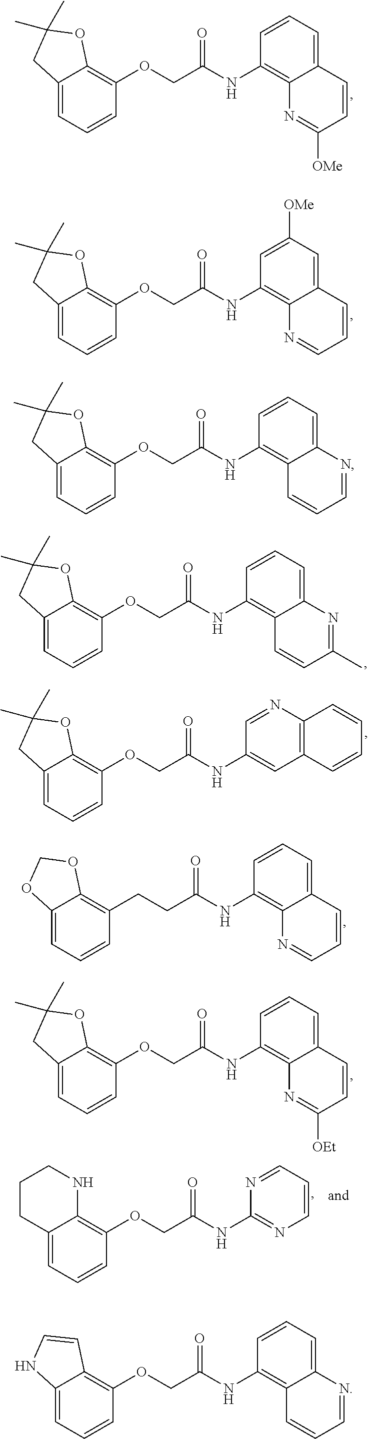

131. A method of treating a disorder or disease in a patient, wherein the disorder or disease is selected from the group consisting of: human immunodeficiency virus 1 (HIV-1), human immunodeficiency virus 2 (HIV-2) and simian immunodeficiency virus (SIV), the method comprising administering to the patient a therapeutically effective amount of a compound, or a pharmaceutically acceptable salt thereof, of Formula III: 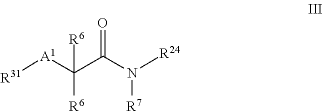
wherein:
R³, R⁴, and R⁵ are independently selected from the group consisting of H, halide, unsubstituted —(C₁₋₆ alkyl), unsubstituted —(C₁₋₄ haloalkyl), —OR¹⁰, and —CN;
each R⁶ is independently selected from the group consisting of H, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein;
R⁷ is selected from the group consisting of H, unsubstituted —(C₁₋₉ alkyl), unsubstituted —(C₃₋₉ alkenyl), unsubstituted —(C₃₋₉ alkynyl), unsubstituted —(C₁₋₉ haloalkyl), —(C₁₋₄ alkylene)C(═O)OR¹¹, —(C₁₋₄ alkylene)C(═O)N(R¹²)₂, —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein;
each R¹⁰ is independently selected from the group consisting of H, unsubstituted —(C₁₋₉ alkyl), unsubstituted —(C₃₋₉ alkenyl), unsubstituted —(C₃₋₉ alkynyl), and unsubstituted —(C₁₋₉ haloalkyl);
R¹¹ is selected from the group consisting of H and unsubstituted —(C₁₋₉ alkyl);
each R¹² is independently selected from the group consisting of H and substituted or unsubstituted —(C₁₋₉ alkyl);
each R¹³ is independently selected from the group consisting of halide, unsubstituted —(C₁₋₉ alkyl), unsubstituted —(C₁₋₉ haloalkyl), and —OR¹⁰;
each R¹⁴ is independently selected from the group consisting of halide, unsubstituted —(C₁₋₉ alkyl), unsubstituted —(C₁₋₉ haloalkyl), and —OR¹⁰;
each R¹⁵ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁶ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁷ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R²⁴ is selected from the group consisting of a 6-membered heteroaryl optionally substituted with 1-4 R²⁵, 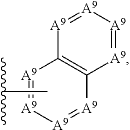
wherein 1-4 A⁹ are N; and 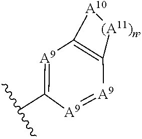
wherein 1-2 A⁹ are N;
each R²⁵ is independently selected from the group consisting of halide, —CN, —OR²⁸, unsubstituted —(C₁₋₆ alkyl), unsubstituted —(C₂₋₆ alkenyl), unsubstituted —(C₂₋₆ alkynyl), unsubstituted —(C₁₋₆ haloalkyl), and carbocyclyl optionally substituted with 1-10 R²⁹;
each R²⁶ is independently selected from the group consisting of H, halide, —CN, —OR²⁸, unsubstituted —(C₁₋₆ alkyl), unsubstituted —(C₂₋₆ alkenyl), unsubstituted —(C₂₋₆ alkynyl), unsubstituted —(C₁₋₆ haloalkyl), and carbocyclyl optionally substituted with 1-10 R²⁹;
each R²⁷ is independently selected from the group consisting of H, halide, —CN, —OR²⁸, unsubstituted —(C₁₋₆ alkyl), unsubstituted —(C₂₋₆ alkenyl), unsubstituted —(C₂₋₆ alkynyl), unsubstituted —(C₁₋₆ haloalkyl), and carbocyclyl optionally substituted with 1-10 R²⁹;
each R²⁸ is independently selected from the group consisting of unsubstituted —(C₁₋₉ alkyl), unsubstituted —(C₃₋₉ alkenyl), unsubstituted —(C₃₋₉ alkynyl), and unsubstituted —(C₁₋₉ haloalkyl);
each R²⁹ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
R³⁰ is selected from the group consisting of H and unsubstituted —(C₁₋₉ alkyl);
R³¹ is selected from the group consisting of 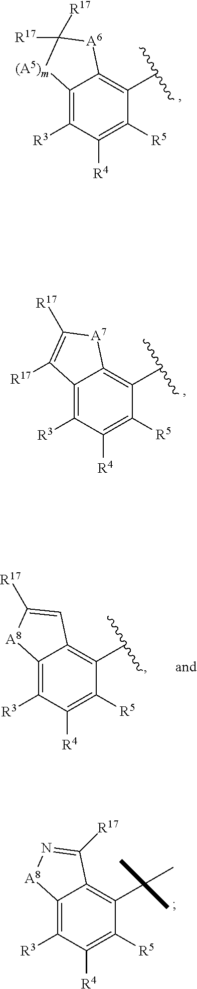
A¹ is selected from the group consisting of —C(R¹⁵)₂—, O, and S;
each A⁵ is independently selected from the group consisting of —C(R¹⁶)₂—, —N(R¹⁰)—, O, and S;
with the proviso that two A⁵ are not both N, O or S;
A⁶ is selected from the group consisting of —C(R¹⁶)₂—, —N(R¹⁰)—, O, and S;
with the proviso that A⁵ and A⁶ cannot both be C(R¹⁶)₂;
A⁷ is selected from the group consisting of —N(R³⁰)—, O and S;
A⁸ is selected from the group consisting of —NH—, O and S;
each A⁹ is independently selected from the group consisting of N and —C(R²⁶)—;
A¹⁰ is selected from the group consisting of —C(R²⁷)₂—, O, and S;
each A¹¹ is independently selected from the group consisting of —C(R²⁷)₂—, O, and S;
with the proviso that no more than two A¹⁰ or A¹¹ are O or S and that two O or S are never adjacent;
n is 1, 2, 3, or 4;
m is 1 or 2; and
each p independently is 0 or 1.

132. A method of treating a disorder or disease in a patient, wherein the disorder or disease is selected from the group consisting of: human immunodeficiency virus 1 (HIV-1), human immunodeficiency virus 2 (HIV-2) and simian immunodeficiency virus (SIV), the method comprising administering to the patient a therapeutically effective amount of a compound, or a pharmaceutically acceptable salt thereof, of Formula IV: 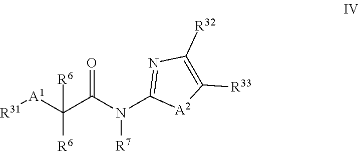
wherein:
R³, R⁴, and R⁵ are independently selected from the group consisting of H, halide, unsubstituted —(C₁₋₆ alkyl), unsubstituted —(C₁₋₄ haloalkyl), —OR¹⁰, and —CN;
each R⁶ is independently selected from the group consisting of H, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
R⁷ is selected from the group consisting of H, unsubstituted —(C₁₋₉ alkyl), unsubstituted —(C₃₋₉ alkenyl), unsubstituted —(C₃₋₉ alkynyl), unsubstituted —(C₁₋₉ haloalkyl), —(C₁₋₄ alkylene)C(═O)OR¹¹, —(C₁₋₄ alkylene)C(═O)N(R¹²)₂, —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein;
each R¹⁰ is independently selected from the group consisting of H, unsubstituted —(C₁₋₉ alkyl), unsubstituted —(C₃₋₉ alkenyl), unsubstituted —(C₃₋₉ alkynyl), and unsubstituted —(C₁₋₉ haloalkyl);
R¹¹ is selected from the group consisting of H and unsubstituted —(C₁₋₉ alkyl);
each R¹² is independently selected from the group consisting of H and substituted or unsubstituted —(C₁₋₉ alkyl);
each R¹³ is independently selected from the group consisting of halide, unsubstituted —(C₁₋₉ alkyl), unsubstituted —(C₁₋₉ haloalkyl), and —OR¹⁰;
each R¹⁴ is independently selected from the group consisting of halide, unsubstituted —(C₁₋₉ alkyl), unsubstituted —(C₁₋₉ haloalkyl), and —OR¹⁰;
each R¹⁵ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁶ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁷ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R³⁰ is selected from the group consisting of H and unsubstituted —(C₁₋₉ alkyl);
R³¹ is selected from the group consisting of 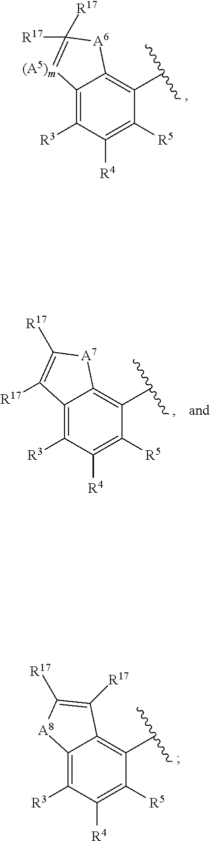
R³² is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -carbocyclyl optionally substituted with 1-10 R³⁴, —C(═O)R³⁵, C(═O)NHR³⁶, and —CN;
R³³ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -carbocyclyl optionally substituted with 1-10 R³⁷, —C(═O)R³⁸, —C(═O)NHR³⁹, and —CN;
each R³⁴ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
R³⁵ is -heterocyclyl optionally substituted with 1-10 R⁴⁰;
R³⁶ is selected from the group consisting of —(C₁₋₄ alkylene)heterocyclyl optionally substituted with 1-10 R⁴¹ and —(C₁₋₄ alkylene)heteroaryl optionally substituted with 1-10 R⁴²; wherein each —(C₁₋₄ alkylene) is, independently, optionally substituted with one or more substituents as defined herein;
each R³⁷ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
R³⁸ is -heterocyclyl optionally substituted with 1-10 R⁴⁴;
R³⁹ is selected from the group consisting of —(C₁₋₄ alkylene)heterocyclyl optionally substituted with 1-10 R⁴⁵ and —(C₁₋₄ alkylene)heteroaryl optionally substituted with 1-10 R⁴⁶; wherein each —(C₁₋₄ alkylene) is, independently, optionally substituted with one or more substituents as defined herein;
each R⁴⁰ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(C₁₋₄ alkylene)ₚOR⁴³; wherein —(C₁₋₄ alkylene) is optionally substituted with one or more substituents as defined herein;
each R⁴¹ is independently selected from the group consisting of halide, unsubstituted —(C₁₋₉ alkyl), and 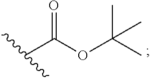 [SMILES: *C(=O)OC(C)(C)C]
each R⁴² is independently selected from the group consisting of halide and unsubstituted —(C₁₋₉ alkyl);
each R⁴³ is independently selected from the group consisting of H and unsubstituted —(C₁₋₉ alkyl);
each R⁴⁴ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(C₁₋₄ alkylene)ₚOR⁴⁷; wherein —(C₁₋₄ alkylene) is optionally substituted with one or more substituents as defined herein;
each R⁴⁵ is independently selected from the group consisting of halide, unsubstituted —(C₁₋₉ alkyl), and 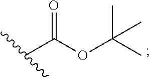 [SMILES: *C(=O)OC(C)(C)C]
each R⁴⁶ is independently selected from the group consisting of halide and unsubstituted —(C₁₋₉ alkyl);
each R⁴⁷ is independently selected from the group consisting of H and unsubstituted —(C₁₋₉ alkyl);
A¹ is selected from the group consisting of —C(R¹⁵)₂—, O, and S;
A² is selected from the group consisting of O and S;
each A⁵ is independently selected from the group consisting of —C(R¹⁶)₂—, —N(R¹⁰)—, O, and S;
with the proviso that two A⁵ are not both N, O or S;
A⁶ is selected from the group consisting of —C(R¹⁶)₂—, —N(R¹⁰)—, O, and S;
with the proviso that A⁵ and A⁶ cannot both be C(R¹⁶)₂;
A⁷ is selected from the group consisting of —N(R³⁰)—, O and S;
A⁸ is selected from the group consisting of —N(R³⁰)—, O and S;
m is 1 or 2;
each p independently is 0 or 1; and
with the proviso that Formula IV is not a structure selected from the group consisting of:

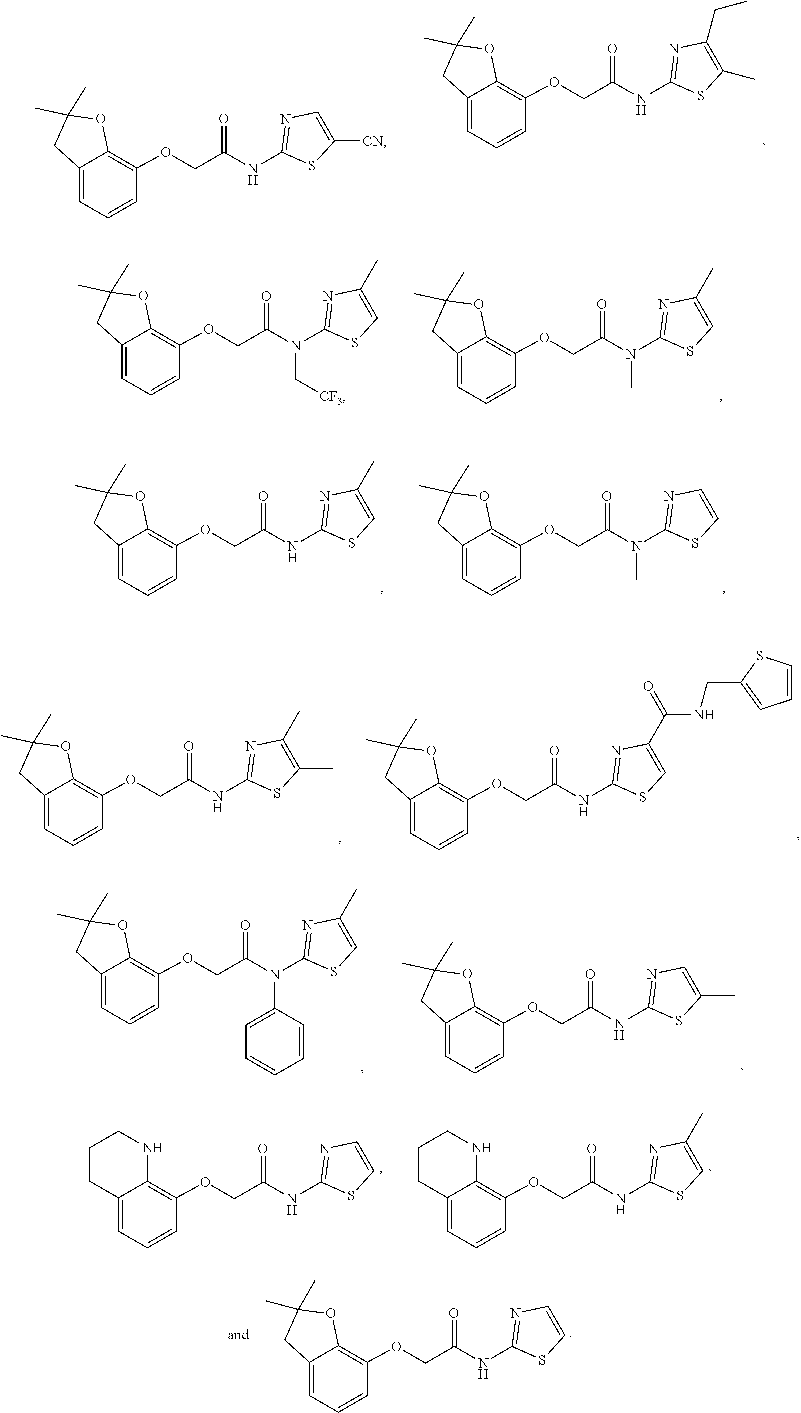

133. A method of treating a disorder or disease in a patient, wherein the disorder or disease is selected from the group consisting of: human immunodeficiency virus 1 (HIV-1), human immunodeficiency virus 2 (HIV-2) and simian immunodeficiency virus (SIV), the method comprising administering to the patient a therapeutically effective amount of a compound, or a pharmaceutically acceptable salt thereof, of Formula IV: 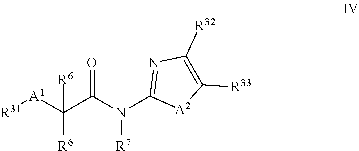
wherein:
R³, R⁴, and R⁵ are independently selected from the group consisting of H, halide, unsubstituted —(C₁₋₆ alkyl), unsubstituted —(C₁₋₄ haloalkyl), —OR¹⁰, and —CN;
each R⁶ is independently selected from the group consisting of H, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
R⁷ is selected from the group consisting of H, unsubstituted —(C₁₋₉ alkyl), unsubstituted —(C₃₋₉ alkenyl), unsubstituted —(C₃₋₉ alkynyl), unsubstituted —(C₁₋₉ haloalkyl), —(C₁₋₄ alkylene)C(═O)OR¹¹, —(C₁₋₄ alkylene)C(═O)N(R¹²)₂, —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein;
each R¹⁰ is independently selected from the group consisting of H, unsubstituted —(C₁₋₉ alkyl), unsubstituted —(C₃₋₉ alkenyl), unsubstituted —(C₃₋₉ alkynyl), and unsubstituted —(C₁₋₉ haloalkyl);
R¹¹ is selected from the group consisting of H and unsubstituted —(C₁₋₉ alkyl);
each R¹² is independently selected from the group consisting of H and substituted or unsubstituted —(C₁₋₉ alkyl);
each R¹³ is independently selected from the group consisting of halide, unsubstituted —(C₁₋₉ alkyl), unsubstituted —(C₁₋₉ haloalkyl), and —OR¹⁰;
each R¹⁴ is independently selected from the group consisting of halide, unsubstituted —(C₁₋₉ alkyl), unsubstituted —(C₁₋₉ haloalkyl), and —OR¹⁰;
each R¹⁵ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁶ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁷ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R³⁰ is selected from the group consisting of H and unsubstituted —(C₁₋₉ alkyl);
R³¹ is selected from the group consisting of 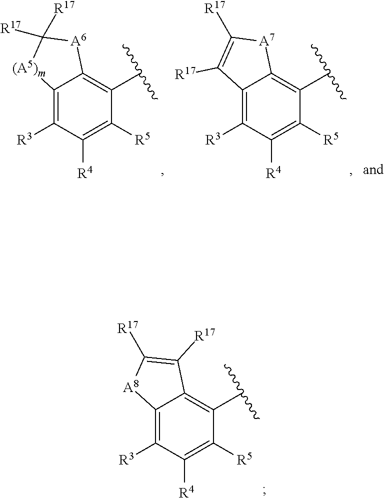
R³² is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -carbocyclyl optionally substituted with 1-10 R³⁴, —C(═O)R³⁵, —C(═O)NHR³⁶, and —CN;
R³³ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -carbocyclyl optionally substituted with 1-10 R³⁷, —C(═O)R³⁸, —C(═O)NHR³⁹, and —CN;
each R³⁴ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
R³⁵ is -heterocyclyl optionally substituted with 1-10 R⁴⁰;
R³⁶ is selected from the group consisting of —(C₁₋₄ alkylene)heterocyclyl optionally substituted with 1-10 R⁴¹ and —(C₁₋₄ alkylene)heteroaryl optionally substituted with 1-10 R⁴²; wherein each —(C₁₋₄ alkylene) is, independently, optionally substituted with one or more substituents as defined herein;
each R³⁷ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
R³⁸ is -heterocyclyl optionally substituted with 1-10 R⁴⁴;
R³⁹ is selected from the group consisting of —(C₁₋₄ alkylene)heterocyclyl optionally substituted with 1-10 R⁴⁵ and —(C₁₋₄ alkylene)heteroaryl optionally substituted with 1-10 R⁴⁶; wherein each —(C₁₋₄ alkylene) is, independently, optionally substituted with one or more substituents as defined herein;
each R⁴⁰ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(C₁₋₄ alkylene)ₚOR⁴³; wherein —(C₁₋₄ alkylene) is optionally substituted with one or more substituents as defined herein;
each R⁴¹ is independently selected from the group consisting of halide, unsubstituted —(C₁₋₉ alkyl), and 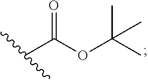 [SMILES: *C(=O)OC(C)(C)C]
each R⁴² is independently selected from the group consisting of halide and unsubstituted —(C₁₋₉ alkyl);
each R⁴³ is independently selected from the group consisting of H and unsubstituted —(C₁₋₉ alkyl);
each R⁴⁴ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(C₁₋₄ alkylene)ₚOR⁴⁷; wherein —(C₁₋₄ alkylene) is optionally substituted with one or more substituents as defined herein;
each R⁴⁵ is independently selected from the group consisting of halide, unsubstituted —(C₁₋₉ alkyl), and 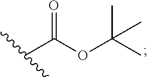 [SMILES: *C(=O)OC(C)(C)C]
each R⁴⁶ is independently selected from the group consisting of halide and unsubstituted —(C₁₋₉ alkyl);
each R⁴⁷ is independently selected from the group consisting of H and unsubstituted —(C₁₋₉ alkyl);
A¹ is selected from the group consisting of —C(R¹⁵)₂—, O, and S;
A² is selected from the group consisting of O and S;
each A⁵ is independently selected from the group consisting of —C(R¹⁶)₂—, —N(R¹⁰)—, O, and S;
with the proviso that two A⁵ are not both N, O or S;
A⁶ is selected from the group consisting of —C(R¹⁶)₂—, —N(R¹⁰)—, O, and S;
with the proviso that A⁵ and A⁶ cannot both be C(R¹⁶)₂;
A⁷ is selected from the group consisting of —N(R³⁰)—, O and S;
A⁸ is selected from the group consisting of —N(R³⁰)—, O and S;
m is 1 or 2; and
each p is 0 or 1.

134. A method of treating a disorder or disease in a patient, wherein the disorder or disease is selected from the group consisting of: human immunodeficiency virus 1 (HIV-1), human immunodeficiency virus 2 (HIV-2) and simian immunodeficiency virus (SIV), the method comprising administering to the patient a therapeutically effective amount of a compound, or a pharmaceutically acceptable salt thereof, of Formula V: 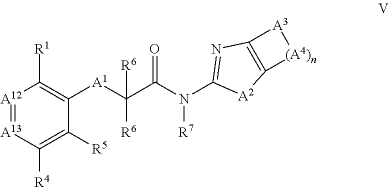
wherein:
R¹ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR⁴⁸, -carbocyclyl optionally substituted with 1-4 R⁴⁹, and —CN;
R² is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR⁴⁸, -carbocyclyl optionally substituted with 1-4 R⁴⁹, and —CN;
R³ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR⁴⁸, -carbocyclyl optionally substituted with 1-4 R⁴⁹, and —CN;
R⁴ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR⁴⁸, -carbocyclyl optionally substituted with 1-4 R⁴⁹, and —CN;
R⁵ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR⁴⁸, -carbocyclyl optionally substituted with 1-4 R⁴⁹, and —CN;
each R⁶ is independently selected from the group consisting of H, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
R⁷ is selected from the group consisting of unsubstituted —(C₁₋₉ alkyl), unsubstituted —(C₃₋₉ alkenyl), unsubstituted —(C₃₋₉ alkynyl), unsubstituted —(C₁₋₉ haloalkyl), —(C₁₋₄ alkylene)C(═O)OR¹¹, —(C₁₋₄ alkylene)C(═O)N(R¹²)₂, —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein;
each R¹⁰ is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
R¹¹ is selected from the group consisting of H and unsubstituted —(C₁₋₉ alkyl);
each R¹² is independently selected from the group consisting of H and substituted or unsubstituted —(C₁₋₉ alkyl);
each R¹³ is independently selected from the group consisting of halide, unsubstituted —(C₁₋₉ alkyl), unsubstituted —(C₁₋₉ haloalkyl), and —OR¹⁰;
each R¹⁴ is independently selected from the group consisting of unsubstituted —(C₁₋₉ alkyl), unsubstituted —(C₁₋₉ haloalkyl), and —OR¹⁰;
each R¹⁵ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁶ is independently selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
each R⁴⁸ is independently selected from the group consisting of unsubstituted —(C₁₋₉ alkyl), unsubstituted —(C₃₋₉ alkenyl), unsubstituted —(C₃₋₉ alkynyl), and unsubstituted —(C₁₋₉ haloalkyl);
each R⁴⁹ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
A¹ is selected from the group consisting of —C(R″)₂—, O, and S;
A² is selected from the group consisting of O and S;
A³ is selected from the group consisting of —C(R¹⁶)₂—, O and S;
each A⁴ is independently selected from the group consisting of —C(R¹⁶)₂—, O and S;
with the proviso that no more than two of A³ and/or A⁴ are O or S and that two O or S are never adjacent;
A¹² is selected from the group consisting of —C(R²)— and N;
A¹³ is selected from the group consisting of —C(R³)— and N;
n is 1 to 4;
p is 0 to 1; and
with the proviso that Formula V is not a structure selected from the group consisting of:

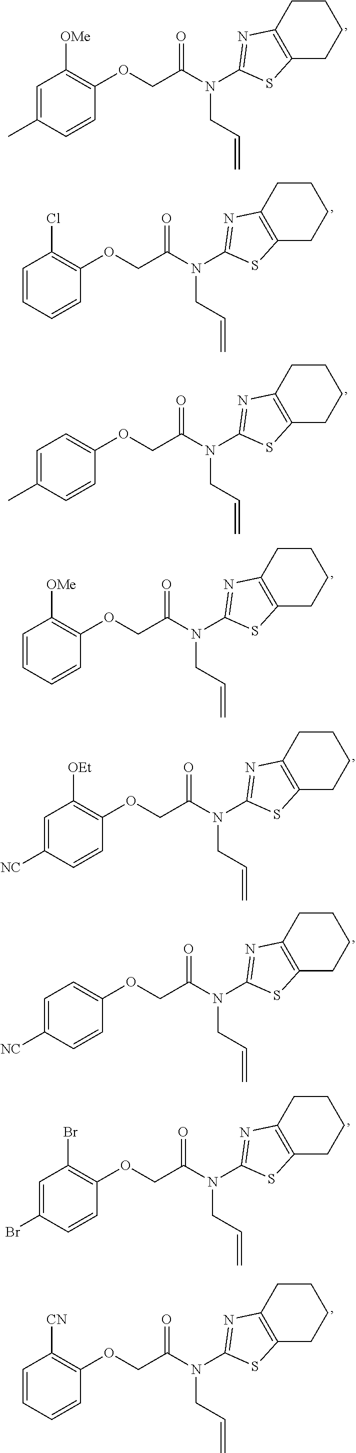

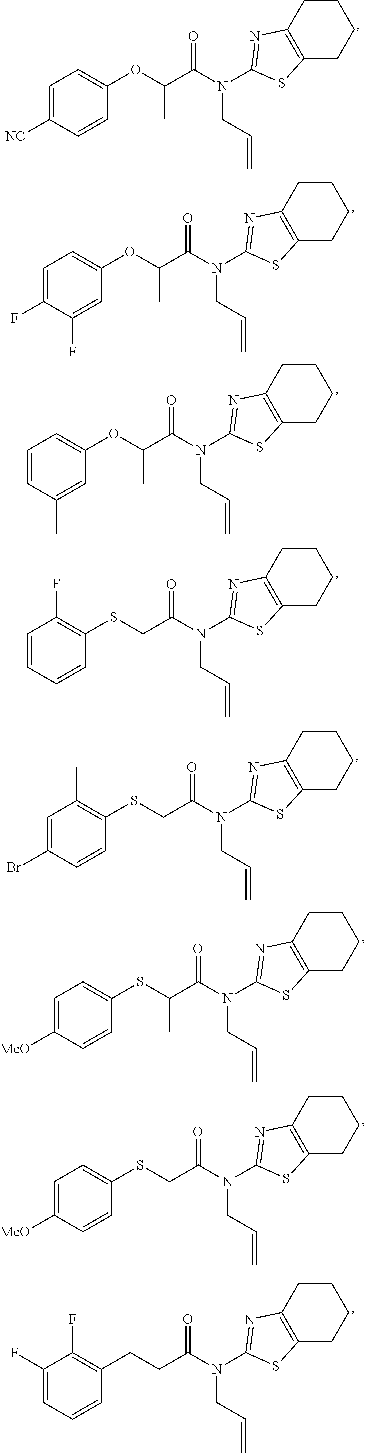

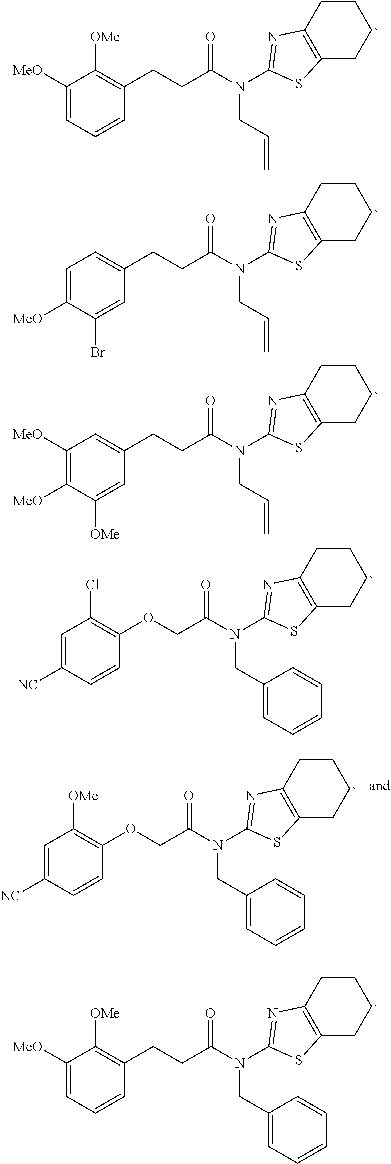

135. A method of treating a disorder or disease in a patient, wherein the disorder or disease is selected from the group consisting of: human immunodeficiency virus 1 (HIV-1), human immunodeficiency virus 2 (HIV-2) and simian immunodeficiency virus (SIV), the method comprising administering to the patient a therapeutically effective amount of a compound, or a pharmaceutically acceptable salt thereof, of Formula V: 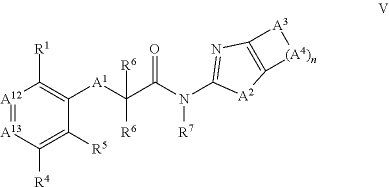
wherein:
R¹ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR⁴⁸, -carbocyclyl optionally substituted with 1-4 R⁴⁹, and —CN;
R² is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR⁴⁸, -carbocyclyl optionally substituted with 1-4 R⁴⁹, and —CN;
R³ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR⁴⁸, -carbocyclyl optionally substituted with 1-4 R⁴⁹, and —CN;
R⁴ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR⁴⁸, -carbocyclyl optionally substituted with 1-4 R⁴⁹, and —CN;
R⁵ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR⁴⁸, -carbocyclyl optionally substituted with 1-4 R⁴⁹, and —CN;
each R⁶ is independently selected from the group consisting of H, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
R⁷ is selected from the group consisting of unsubstituted —(C₁₋₉ alkyl), unsubstituted —(C₁₋₉ haloalkyl), —(C₁₋₄ alkylene)C(═O)OR¹¹, —(C₁₋₄ alkylene)C(═O)N(R¹²)₂, —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein;
each R¹⁰ is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
R¹¹ is selected from the group consisting of H and unsubstituted —(C₁₋₉ alkyl);
each R¹² is independently selected from the group consisting of H and substituted or unsubstituted —(C₁₋₉ alkyl);
each R¹³ is independently selected from the group consisting of halide, unsubstituted —(C₁₋₉ alkyl), unsubstituted —(C₁₋₉ haloalkyl), and —OR¹⁰;
each R¹⁴ is independently selected from the group consisting of unsubstituted —(C₁₋₉ alkyl), unsubstituted —(C₁₋₉ haloalkyl), and —OR¹⁰;
each R¹⁵ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁶ is independently selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
each R⁴⁸ is independently selected from the group consisting of unsubstituted —(C₁₋₉ alkyl), unsubstituted —(C₃₋₉ alkenyl), unsubstituted —(C₃₋₉ alkynyl), and unsubstituted —(C₁₋₉ haloalkyl);
each R⁴⁹ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
A¹ is selected from the group consisting of —C(R¹⁵)₂—, O, and S;
A² is selected from the group consisting of O and S;
A³ is selected from the group consisting of —C(R¹⁶)₂—, O and S;
each A⁴ is independently selected from the group consisting of —C(R¹⁶)₂—, O and S;
with the proviso that no more than two of A³ and/or A⁴ are O or S and that two O or S are never adjacent;
A¹² is selected from the group consisting of —C(R²)— and N;
A¹³ is selected from the group consisting of —C(R³)— and N;
n is 1, 2, 3, or 4; and
p is 0 or 1.

136. A method of treating a disorder or disease in a patient, wherein the disorder or disease is selected from the group consisting of: human immunodeficiency virus 1 (HIV-1), human immunodeficiency virus 2 (HIV-2) and simian immunodeficiency virus (SIV), the method comprising administering to the patient a therapeutically effective amount of a compound, or a pharmaceutically acceptable salt thereof, of Formula VI: 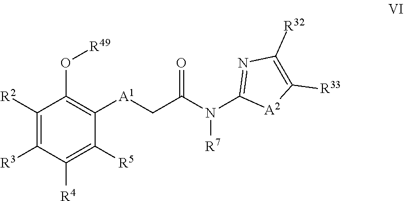
wherein:
R² is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R³ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R⁴ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R⁵ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R⁷ is selected from the group consisting of H, unsubstituted —(C₁₋₉ alkyl), unsubstituted —(C₃₋₉ alkenyl), unsubstituted —(C₃₋₉ alkynyl), unsubstituted —(C₁₋₉ haloalkyl), —(C₁₋₄ alkylene)C(═O)OR¹¹, —(C₁₋₄ alkylene)C(═O)N(R¹²)₂, —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein;
each R¹⁰ is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
R¹¹ is selected from the group consisting of H and unsubstituted —(C₁₋₉ alkyl);
each R¹² is independently selected from the group consisting of H and substituted or unsubstituted —(C₁₋₉ alkyl);
each R¹³ is independently selected from the group consisting of halide, unsubstituted —(C₁₋₉ alkyl), unsubstituted —(C₁₋₉ haloalkyl), and —OR¹⁰;
each R¹⁴ is independently selected from the group consisting of halide, unsubstituted —(C₁₋₉ alkyl), unsubstituted —(C₁₋₉ haloalkyl), and —OR¹⁰;
each R¹⁵ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
R³² is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R³³ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R⁴⁹ is selected from the group consisting of unsubstituted —(C₃₋₉ alkyl), unsubstituted —(C₃₋₉ alkenyl), unsubstituted —(C₃₋₉ alkynyl), unsubstituted —(C₃₋₉ haloalkyl), —(C₁₋₄ alkylene)ₚheteroaryl optionally substituted with 1-4 R⁵⁰, and —(C₁₋₄ alkylene)ₚaryl optionally substituted with 1-5 R⁵¹, and —(C₁₋₄ alkylene)ₚcarbocyclyl optionally substituted with 1-10 R⁵²; wherein each —(C₁₋₄ alkylene) is, independently, optionally substituted with one or more substituents as defined herein;
each R⁵⁰ is independently selected from the group consisting of halide, unsubstituted —(C₁₋₉ alkyl), unsubstituted —(C₁₋₉ haloalkyl), and —OR¹⁰;
each R⁵¹ is independently selected from the group consisting of halide, unsubstituted —(C₁₋₉ alkyl), unsubstituted —(C₁₋₉ haloalkyl), and —OR¹⁰;
each R⁵² is independently selected from the group consisting of halide, unsubstituted —(C₁₋₉ alkyl), and unsubstituted —(C₁₋₉ haloalkyl);
A¹ is selected from the group consisting of —C(R″)₂—, O, and S;
A² is selected from the group consisting of O and S;
each p independently is 0 or 1; and
with the proviso that Formula VI is not:

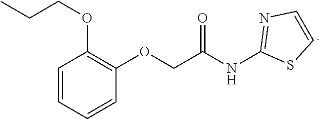 [SMILES: CCCOc1ccccc1OCC(=O)Nc1nccs1]

137. A method of treating a disorder or disease in a patient, wherein the disorder or disease is selected from the group consisting of: human immunodeficiency virus 1 (HIV-1), human immunodeficiency virus 2 (HIV-2) and simian immunodeficiency virus (SIV), the method comprising administering to the patient a therapeutically effective amount of a compound, or a pharmaceutically acceptable salt thereof, of Formula VI: 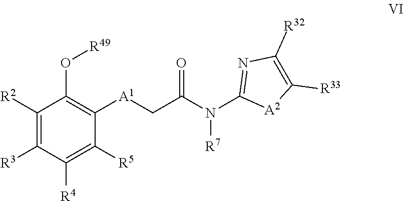
wherein:
R² is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R³ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R⁴ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R⁵ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R⁷ is selected from the group consisting of H, unsubstituted —(C₁₋₉ alkyl), unsubstituted —(C₃₋₉ alkenyl), unsubstituted —(C₃₋₉ alkynyl), unsubstituted —(C₁₋₉ haloalkyl), —(C₁₋₄ alkylene)C(═O)OR¹¹, —(C₁₋₄ alkylene)C(═O)N(R¹²)₂, —(C₁₋₄ alkylene)ₚheteroaryl optionally substituted with 1-4 R¹³, and —(C₁₋₄ alkylene)ₚaryl optionally substituted with 1-5 R¹⁴; wherein each —(C₁₋₄ alkylene) is, independently, optionally substituted with one or more substituents as defined herein;
each R¹⁰ is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
R¹¹ is selected from the group consisting of H and unsubstituted —(C₁₋₉ alkyl);
each R¹² is independently selected from the group consisting of H and substituted or unsubstituted —(C₁₋₉ alkyl);
each R¹³ is independently selected from the group consisting of halide, unsubstituted —(C₁₋₉ alkyl), unsubstituted —(C₁₋₉ haloalkyl), and —OR¹⁰;
each R¹⁴ is independently selected from the group consisting of halide, unsubstituted —(C₁₋₉ alkyl), unsubstituted —(C₁₋₉ haloalkyl), and —OR¹⁰;
each R¹⁵ is independently selected from the group consisting of H, halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
R³² is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R³³ is selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —CN;
R⁴⁹ is selected from the group consisting of unsubstituted —(C₃₋₉ alkyl), unsubstituted —(C₃₋₉ alkenyl), unsubstituted —(C₃₋₉ alkynyl), unsubstituted —(C₃₋₉ haloalkyl), —(C₁₋₄ alkylene)ₚheteroaryl optionally substituted with 1-4 R⁵⁰, and —(C₁₋₄ alkylene)ₚaryl optionally substituted with 1-5 R⁵¹, and —(C₁₋₄ alkylene)ₚcarbocyclyl optionally substituted with 1-10 R⁵²; wherein each —(C₁₋₄ alkylene) is, independently, optionally substituted with one or more substituents as defined herein;
with the proviso that when R⁷ is H, then R⁴⁹ is not —(C₃ alkyl);
each R⁵⁰ is independently selected from the group consisting of halide, unsubstituted —(C₁₋₉ alkyl), unsubstituted —(C₁₋₉ haloalkyl), and —OR¹⁰;
each R⁵¹ is independently selected from the group consisting of halide, unsubstituted —(C₁₋₉ alkyl), unsubstituted —(C₁₋₉ haloalkyl), and —OR¹⁰;
each R⁵² is selected from the group consisting of halide, unsubstituted —(C₁₋₉ alkyl), and unsubstituted —(C₁₋₉ haloalkyl);
A¹ is selected from the group consisting of —C(R¹⁵)₂—, O, and S;
A² is selected from the group consisting of O and S; and
each p independently is 0 or 1.

138. A method of treating a disorder or disease in a patient, wherein the disorder or disease is selected from the group consisting of: human immunodeficiency virus 1 (HIV-1), human immunodeficiency virus 2 (HIV-2) and simian immunodeficiency virus (SIV), the method comprising administering to the patient a therapeutically effective amount of a compound, or a pharmaceutically acceptable salt thereof, according to Table 1 or Table 2.

139. A method according to any one of claims 126-138, wherein the disorder or disease is HIV-1.

140. The method of claim any one of claims 126-138, wherein the patient is a human.
